(19) United States
(12) Patent Application Publication
(10) Pub. No.: US 2006/0275905 A1
(43) Pub. Date: Dec. 7, 2006

(54) Gram positive bacterial mutants and methods of generating and using such mutants

(75) Inventors: Taeok Bae, Chicago, IL (US); Fredrik Aslund, Rijswijk (NL); Olaf Schneewind, Chicago, IL (US); Dominique M. Missiakas, Chicago, IL (US)

Correspondence Address: FULBRIGHT & JAWORSKI L.L.P., 600 CONGRESS AVE., SUITE 2400, AUSTIN, TX, 78701, US

(73) Assignee: The University of Chicago

(21) Appl. No.: 11/189,065

(22) Filed: Jul. 25, 2005

Related U.S. Application Data
(60) Provisional application No. 60/591,007, filed on Jul. 26, 2004.

(51) Int. Cl. A61K 49/00; C12N 15/74; C12N 1/21

(52) U.S. Cl. 435/473; 435/252.3; 424/9.2

(57) ABSTRACT

The present invention concerns gram positive bacterial mutants, particularly staphylococcus mutants of the Newman strain. It also involves transposable elements that can be used to generate these mutants, as well as methods of creating such mutants with these elements. The inventions also includes methods of using the bacterial mutants to screen for different types of genes, including virulence genes, which can lead to assays for drug compounds useful in the treatment of bacterial infection.

This application claims priority to U.S. Provisional Patent Application Ser. No. 60/591,007 filed on Jul. 26, 2004, which is hereby incorporated by reference in its entirety.

### BACKGROUND OF THE INVENTION

1. Field of the Invention

The present invention relates generally to the fields of microbiology and pathology. More particularly, it concerns methods and compositions involving Staphylococcus aureus mutants.

2. Description of Related Art

Previous work has been done on Staphylococcus aureus to identify staphylococcal virulence factors using Tn917 and signature tagged mutagenesis (Hartley et al., 1986; Mei et al., 1997). Libraries of 1248 or 1520 randomly chosen (non-sequenced) transposon insertion mutants of S. aureus were analyzed in animal infections with mixed populations to reveal a competitive disadvantage of individual variants. Based on the distribution of Tn917 knockout mutations that were analyzed here, it is estimated that these studies examined approximately 400-500 S. aureus genes. Only about 20% of all S. aureus genes were analyzed; such studies provide important forays into defining the roles of specific S. aureus genes during the pathogenesis of specific infectious diseases (Mei et al., 1997).

Gene function in S. aureus has been previously examined using antisense technology (Ji et al., 2001; Forsyth et al., 2002). By cloning gene fragments in reversed orientation under control of an inducible promoter, the ability of antisense nucleotide sequences to interfere with S. aureus growth on agar plates was used to identify essential genes. In two independent studies, only 30% of all gene sequences (350 genes) in each study identified the same gene (75 genes total) (Ji et al., 2001; Forsyth et al., 2002).

However, a mutagenesis scheme that generates mutations throughout the S. aureus genome and a more thorough analysis of such mutants are needed to gain a further understanding of S. aureus virulence and to identify new therapeutic and preventative agents.

### SUMMARY OF THE INVENTION

The present invention concerns gram positive bacterial mutants, as well as their production and use. It is based on the creation of a transposable nucleic acid element that can be used in gram positive bacteria, particularly Staphlococcus bacteria, to generate mutations, including mutations in virulence genes. These mutants can be screened for virulence genes, as well as other classes of genes, to engage in further study and experimentation. Thus, the present invention encompasses nucleic acid elements, bacterial mutants, production methods, screening methods, and therapeutic methods.

In some embodiments, the invention covers a transposable nucleic acid element, which can be incorporated into the genome of a heterologous organism, such as a gram positive bacterium. Particularly contemplated is an element comprising a pair of inverted repeat sequences recognized by a Himar 1 transposase, wherein an inverted repeat sequence is at each end of the element (referred to as “Himar 1 element”). The element may contain other nucleic acid sequences. In further embodiments, the element has, but is not limited to, at least one screenable marker gene. The element may have, have at least, or have at most 1, 2, 3, 4, 5, 6 or more screenable marker genes, as well as promoters or other control elements for expression of polypeptides that may be encoded by the sequences. Such screenable markers may encode for polypeptides that are calorimetric, fluorescent, or enzymatic. In certain cases, the screenable marker could be a selectable marker gene. Furthermore, an element may contain one or more selectable marker genes and/or one or more non-selectable (but screenable) marker genes. In some cases, the element has a selectable marker gene that encodes a polypeptide conferring antibiotic resistance. The resistance can be, but is not limited to, an antibiotic selected from the group consisting of erythromycin, tetracycline, spectinomycin, and chloramphenicol. In specific embodiments, the antibiotic is erythromycin or tetracycline.

The screenable marker can be used to identify an organism in which the element has been incorporated intrachromosomally or episomally. In particular cases, it can be used to identify organisms that have the element inserted into a chromosome. Alternatively, a screenable element can be used to identify or characterize nucleic acid control sequences near or at the site of integration. In some instances a screenable marker lacks a promoter so as to identify or characterize a nucleic acid sequence at the site of integration that provides a promoter sequence. The identification of enhancers can similarly be implemented. In some embodiments, the screenable marker is a gene encoding a colorimetric polypeptide, such as a green fluorescent protein.

The present invention specifically includes the transposable element termed Bursa aurealis, which refers to a transposable element having 1) an inverted repeat sequence in which there is the sequence

(SEQ ID NO:4)

5′-ACAGGTTGGCTGATAAGTCCCCGGTCTCTAGAG-3′

toward the 5′ end of the element (“toward the end” means that a portion of the element is within 50 nucleotides of the end) and the sequence

(SEQ ID NO:5)

5′-GTCTAGAGACCGGGG ACTTATCAGCCAACCTGT-3′

is downstream toward the 3′ end of the element; and 2) the ermC resistance marker. In certain embodiments, it also has 3) a green fluorescent protein gene without a promoter. It is contemplated that SEQ ID NO:4 and/or SEQ ID NO:5 may be placed into other elements with one or more functional genes, such as a selectable or screenable marker. Moreover, in some embodiments, the sequence of Bursa aurealis is SEQ ID NO:3, which contains both SEQ ID NO:4 and SEQ ID NO:5. One of the sequences in the pair of inverted repeat sequences (SEQ ID NO:4) is toward or at the end of the element while the other sequence in the pair of inverted repeat sequences (SEQ ID NO:5) is toward or at the other end of the element. It is contemplated that concomitant changes (changes in both sequences) may be made and still enable the pair to be used as a transposable element recognized by the Himar 1 transposase.

Nucleic acids of the invention can have, have at most or have at least the following number of contiguous nucleotides from SEQ ID NO:1, SEQ ID NO:2, SEQ ID NO:3, SEQ ID NO:4, and SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 31, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more, or any range derivable therein. Nucleic acids of the invention also include the full-lengths of SEQ ID NO:1, SEQ ID NO:2, SEQ ID NO:3, SEQ ID NO:4, and SEQ ID NO:5. Bacteria of the invention may have all or part of these SEQ ID NOs as well.

In certain embodiments, the transposable element is contained within a plasmid or other vector. In certain embodiments, the plasmid of vector also includes an origin of replication, which comprises a temperature-sensitive replicon. The term “temperature-sensitive replicon” connotes a region that controls the replication of the plasmid or vector in a temperature-sensitive manner. There will be a temperature range at which replication can occur (permissive temperature) and a higher temperature at which replication no longer occurs (non-permissive temperature).

In certain embodiments of the invention, there is a nucleic acid encoding a transposase that recognizes and transposes the transposable element of the invention. The transposase is a hornfly transposase, meaning it is a transposase derived from the hornfly. In certain embodiments, the transposase is hornfly-based, meaning it is derived from the hornfly yet has been recombinantly altered thereafter (but has the same or better activity).

In some examples of the invention, there is a plasmid or vector containing a transposable element of the invention and/or a nucleic acid encoding a transposase that recognizes the element. Such vectors can be in a bacterium either to propagate the vector or as the target of transposon-induced mutagenesis. In certain cases, the bacterial host is, but is not limited to, gram-positive bacteria, such as staphylococcus bacteria. Moreover, both the vector containing the transposable element and a vector containing the cognate transposase may be in the same bacterial cells together. The term “cognate” transposase in the context of a transposable element refers to the transposase that mediates transposition of the transposable element. Accordingly, in some embodiments of the invention, there is a bacterium with a Himar 1 element and/or a nucleic acid encoding a Himar 1 transposase.

In some embodiments, the invention concerns host cells that contain the Himar 1 element, a nucleic acid encoding a Himar 1 transposase, or both. It is contemplated that the Himar 1 element may be on a separate nucleic acid molecule than the one encoding the transposase, though in some cases they are contained in the same nucleic acid molecule, such as a plasmid. In certain embodiments, the Himar 1 element is on a plasmid in the host cell. Moreover, the nucleic acid may also be contained in a plasmid in the host cell. A host cell with an exogenous nucleic acid is typically considered a recombinant host cell. As discussed throughout the application, the host cell may be a bacterium. In certain cases, the bacterium is a gram-positive bacterium.

The invention further encompasses bacteria, particularly gram-positive bacterium having a transposon insertion, which means the bacterium has at least one element that was transposed into its genome. The insertion could be random or pre-determined or engineered. It is specifically contemplated that in some embodiments the element is a Himar 1 element. Moreover, embodiments of the invention concern, but are not limited to, the gram-positive bacteria Staphylococcus aureus. Particularly contemplated is the Newman strain of S. aureus bacteria. Bacteria containing any of the transposable elements and embodiments discussed above and those identified in Tables 1, 2, and 3 are contemplated as part of the invention. In certain embodiments, a bursa aurealis isolate is the invention, which refers to a bacterium with a bursa aurealis insertion in its genome.

Bacteria with different mutations can be created using the transposable elements of the invention. The transposon may insert itself in any site throughout the genome and is not limited to any particular place. Each individual bacterium has a mutation in a specific location. In some embodiments, the insertion is in a gene selected from the group consisting of: metabolic gene, regulatory gene, extracellular factor gene, cellular or secreted gene, and any putative gene based on the presence of an ORF and/or conservation of sequence with other organisms (“hypothetical gene” or “conserved hypothetical gene”). Alternatively, the insertion may be in a gene encoding an enzyme, a structural protein, a membrane protein, transporter, symporter, or a functional RNA molecule (such as an rRNA, tRNA, tmRNA, or small RNA), and any other gene in the bacterial genome, including any of those identified in Tables 2 or 3.

In some cases, a transposon will be inserted into the genome so as to negatively or to positively affect virulence. The term “negatively affects virulence” means that the bacterial host with a transposon insertion is less virulent compared to the same bacterial host lacking the insertion, according to any virulence assay used by those of skill in the art, including those discussed in the Examples. An insertion that “negatively affects virulence” can be one that, for example, eliminates virulence, reduces infectivity, increases an immune response to the bacterial host, reduces the number of nematodes killed, or reduces the amount or extent of abscess formation in organs of animal models of infection. Similarly, the term “positively affect virulence” means that the bacterial host with a transposon insertion is more virulent compared to the same bacterial host lacking the insertion, according to any virulence assay used by those of skill in the art, including those discussed in the Examples. An insertion that “positively affects virulence” can be one that, for example, increases or enhances virulence, increases infectivity, decreases an immune response to the bacterial host, increases the number of nematodes killed, or increases the amount or extent of abscess formation in organs of animal models of infection.

Any embodiment concerning transposable elements can be used in any method of the invention, and vice versa. Methods of the invention involve using transposable elements and bacteria having an element transposed in their genome.

Methods of the invention also concern methods for generating one or more gram positive bacterial mutants using transposable elements of the invention. While bacteria other than gram-positive bacteria can be the subject of aspects of the invention, the use of gram positive bacteria is specifically contemplated. In certain embodiments, a method comprises: a) introducing into a gram positive bacterium i) a nucleic acid comprising a transposable element comprising a screenable marker gene and a pair of inverted repeats recognized by a Himar 1 transposase at each end of the element and ii) a gene encoding a Himar 1 transposase, wherein the gene is under the control of a promoter that expresses the transposase in the bacterium; and b) screening the bacterium using the phenotype conferred by the screenable marker gene, wherein integration of the transposable element creates a genetic mutation. In specific embodiments nucleic acids are introduced in a bacterium by transformation or tranduction. Furthermore, in certain other embodiments, the screenable marker gene is a selectable marker gene and the screening involves selecting the bacterium using the phenotype conferred by the selectable marker gene. This method can involve a situation in which the nucleic acid comprising the transposable element is a plasmid that also includes a temperature-sensitive gram positive replicon. In these embodiments, the method can further include a step of raising the temperature to a nonpermissive temperature of the replicon between steps a) and b).

In further embodiments, a mutation can be characterized by functional phenotype. Therefore, aspects of the invention concern identifying a transposon insertion mutation in a virulence gene. A “virulence gene” is a gene needed at least specifically for the bacteria to be virulent but is distinguishable from a gene required simply for the viability of the bacteria. For example, certain structural proteins are needed for the bacteria to survive, so in some respect they are needed for virulence, but they would not qualify as virulence genes unless a bacterium could live without them but its ability to be virulent was affected. Typically, the gene product, as opposed to the gene itself, is involved in the virulence of the bacteria. Any embodiment concerning transposable elements can be used in any method of the invention, and vice versa. Virulence can be measured or characterized by a number of different assays, which are well known to those of skill in the art. In certain cases, a virulence gene is identified or characterized using a nematode killing assay, which is described in the Examples. Alternatively or in addition to, a virulence gene can be identified or characterized by infecting a mouse or other mammal with a mutant having a mutation identified using the nematode killing assay and analyzing the animal for an organ abscess or organ abscesses.

Methods of the invention include a method of identifying a gram positive bacterium having a mutation in a putative virulence gene. Methods can include one or more of the following: a) obtaining a gram positive bacteria with a transposon insertion, wherein the transposon insertion comprises a pair of inverted repeats recognized by a Himar 1 transposase; b) incubating the bacteria with nematodes on an agar plate; and, c) assaying for nematode killing, wherein reduced nematode killing compared to nematode killing by gram positive bacteria without a transposon insertion identifies a bacterium with a mutation in a putative virulence gene. With this method as well as any other method of the invention, it is contemplated that a comparison can be made using a control either assayed at the same time or at a previous time. A control value may be configured based on one or more previous experiments, in which case the value may be a standardized or normalized value with or without a standard deviation. Moreover, the term “nematode killing” refers to the number of nematodes killed when incubated with the bacteria.

Other methods of the invention concern identifying or characterizing a virulence gene in a gram positive bacterium comprising: a) incubating with nematodes on an agar plate a gram positive bacterium with a transposon insertion in a putative virulence gene, wherein the transposon insertion comprises a pair of inverted repeats recognized by a Himar 1 transposase; and, b) assaying for nematode killing, wherein reduced nematode killing compared to nematode killing by gram positive bacteria without a transposon insertion is indicative of an insertion in a putative virulence gene. The term “transposon insertion” means that there is a transposon inserted in the genome of the bacterium.

Methods of the invention also concern a method of screening for a virulence gene in a gram positive bacteria comprising: a) obtaining a gram positive bacteria with a transposon insertion, wherein the transposon insertion comprises a pair of inverted repeats recognized by a Himar 1 transposase; b) infecting a mouse or other host mammal with the bacteria having a transposon insertion and assaying the animal for organ abscesses, wherein a reduced amount of organ abscesses compared to a mouse incubated with the gram positive bacteria without a transposon insertion is indicative of an insertion in a putative virulence gene.

Methods of the invention involve, in some embodiments, identifying the location of an insertion that creates a genetic mutation. This can be done by a number of methods, including sequencing a region on one or both sides of the integrated transposable element.

In some embodiments, methods involve identifying the putative virulence gene. Additional assays or experiments may be conducted to characterize further or confirm the identification of a virulence gene. Moreover, it can also include screening for an inhibitor of the identified putative virulence gene.

Embodiments of the invention also concern a population of different bacterial mutants, referred to as a “pool.” A “pool” is understood to refer to 2 or more different mutants of the same strain and type of bacteria, such as in the same gram positive bacteria. Embodiments of the invention include a pool of S. aureus Newman strain mutants. The term “different bacterial mutants” refers to bacteria each having a different mutation(s). In certain cases the pool includes, includes at least, or includes at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10,000, 10,325, 11,000, 12,000, 13,000 14,000, 15,000 or more different bacterial mutants, or any range derivable therein. In certain embodiments, a pool contains between 2 and 500, between 5 and 400, between 10 and 200, or between 50 and 100 bacteria, each with a different transposon insertion. It is understood that a pool of different mutants can be referred to as a collection of mutants. Table 3 identifies such a pool or collection of mutants. It is contemplated that any pool or collection of the invention may contain all or part of the list of mutants in any of Tables 1, 2, and/or 3. In certain embodiments, the pool or collection contains any of the numbers of different bacterial mutants described above, including any range derivable therein.

In some methods, there is a step of lysing a bacteria with a transposon insertion in a putative virulence gene; transducing the insertion into a gram positive bacterium lacking a transposon insertion; and, evaluating the transduced bacteria with a nematode killing assay, wherein reduced killing compared to gram positive bacteria without a transposon insertion is further indicative of an insertion in a putative virulence gene. Furthermore, methods may also include infecting a mouse or other mammal with the bacteria having a transposon insertion in a putative virulence gene and assaying the mouse for organ abscesses, wherein a reduced amount of organ abscesses compared to an animal incubated with gram positive bacteria without a transposon insertion is further indicative of an insertion in a putative virulence gene.

It is also contemplated that screening assays may be conducted with a pool or population of bacterial mutants to expedite screening methods. If a pool or population of mutants yields reduced nematode killing, individual mutants or a subset of mutants in the pool can be assayed in methods of the invention to identify mutants giving rise to the reduced nematode killing. Putative virulence genes can be identified in this manner.

Moreover, methods and compositions of the invention can be readily applied to screening for, identifying, and characterizing other types of bacterial genes. Such screening methods are implemented like the screening methods for virulence genes, except the screen involves assaying for the activity, function, or other phenotype caused, attributed to or related to that particular gene or type of gene.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

It is specifically contemplated that any embodiments described in the Examples section are included as an embodiment of the invention.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Plasmids used for transposon mutagenesis in S. aureus. Bursa aurealis, a mini-mariner transposable element, was cloned into pTS2, with a temperature-sensitive plasmid replicon (repₜₛ) and chloramphenicol resistance gene (cat) to generate pBursa; the 3.2 kb bursa aurealis encompasses the mariner terminal inverted repeats (TIR), the green fluorescent protein gene (gfp), the R6K replication origin (oriV) and erythromycin resistance determinant ermB. The position of restriction enzyme recognition sites (AciI and BamHI) is indicated. Plasmid pFA545 encodes the mariner transposase (tnp), tetracycline (tetB tetD), ampicillin (bla) resistance markers, and origin of replication (repC). Shown at the bottom of the figure is the structure of the 5.3 kb transposon Tn917 with ermB erythromycin resistance determinant, tnpR resolvase and tnpA transposase.

FIG. 2. Comparison of the transposon insertion sites of bursa aurealis and Tn917. S. aureus strain Newman was subjected to mutagenesis and 582 insertion mutants of each transposon were mapped by DNA sequencing and aligned according to their insertion site on the genome of S. aureus strain Mu50, which is available in the art.

FIG. 3. Bursa aurealis transposon insertions in the genome of S. aureus strain Newman. Four regions of the genome, in which very few transposon mutants were isolated, are indicated by blue bars with the names of the genes residing in each of the four regions. The first circle represents open reading frames that were disrupted (black) or not disrupted (white) by bursa aurealis. The second and third circles represent open reading frames in the genome of S. aureus strains Mu50, in which different colors were assigned according to their functions. Green and red arrows indicate tRNAs and rRNA, respectively. The fourth circle shows the scale of the map (bp). The fifth circle is a circular bar graph showing the number of transposon mutants per open reading frame (scale bar with each ladder step representing 10 mutants). Sccmec, staphylococcal cassette chromosome mec and SaPIn1, staphylococcal pathogenicity island 1, are indicated. Bursa aurealis insertions (116) were isolated at the same position within thiD, but these insertions are likely to represent siblings as the isolated mutants arose from the same batch of transposon insertion mutants.

FIGS. 4A-B. Identification of S. aureus virulence genes in a mouse model of infection. S. aureus Newman or bursa aurealis insertion mutants ΦNΞ12478 (FIG. 4A) or ΦNΞ02863 (FIG. 4B) were used to infect ten mice each via retro-orbital injection (˜1.3×10⁷ CFU). After four days, mice were euthanized and organs (kidney, liver and spleen) removed. Homogenized tissues were incubated on agar medium for S. aureus colony formation and enumeration. Each circle indicates an animal experiment. The dashed line represents the limit of detection, P designates the p value and horizontal bars indicate the mean.

### DESCRIPTION OF ILLUSTRATIVE EMBODIMENTS

The present invention concerns transposable elements, their use in generating genomic mutations, the mutant bacteria, and methods of using and screening the mutant bacteria. In particular embodiments, the invention concerns gram positive bacteria, such as Staphylococcus aureus.

I. Staphylococcus aureus

The gram-positive bacterium S. aureus is a physiological commensal of the human skin, nares and mucosal surfaces (Lowy 1998; Archer 1998). Staphylococci are also opportunistic and adaptable pathogens equipped with the ability to infect, invade, persist and replicate in any human tissue including skin, bone, visceral organs or vasculature (Lowy 1998; Gillet et al., 2002). As a result of adaptation to specific tissues, staphylococci cause many diverse pathological syndromes such as abscess, bacteremia, osteomyelitis, toxic shock syndrome, food poisoning and endocarditis (Lowy 1998; Archer et al. 2001). Because they colonize the human skin continuously, S. aureus strains are exposed to all antibiotic therapies (Neu 1992). Whenever drug-resistant microbes emerge, these strains can spread by direct contact very rapidly among human populations, as is exemplified by the threat of methicillin-resistant S. aureus (MRSA) and vancomycin-resistant S. aureus (VRSA) in communities and hospitals world-wide (Brumfitt et al., 1989; Hiramatsu et al., 1997; Chang et al., 2003; Weigel et al., 2003; Oliveira et al, 2002).

Antibiotic therapy has focused on targets that are required by all bacteria, for example cell wall synthesis, protein synthesis and DNA replication (Walsh 2000). This therapeutic strategy cannot distinguish between resident non-pathogenic flora and invading pathogens and, in principle, does not require identification of etiologic disease agents or specific diagnosis to commence therapy (Alksne et al., 2000). As bacteria distribute genetic information beyond species boundaries, the emergence of drug-resistant microbes, even those that are unable to cause human disease, profoundly impacts the selection of pathogenic microbes with resistance mechanisms against all currently known antibiotic therapies (Alksne et al., 2000).

The 2.7-2.9 Mb genomes of several different S. aureus strains have been sequenced and encompass between 2,550 and 2,870 different genes (Kuroda et al., 2001; Baba et al., 2002; Fitzgerald et al., 2001). Over the past several decades, S. aureus secreted toxins, surface proteins and regulatory factors were identified as virulence factors and characterized, often using reverse genetic approaches (Archer 1998). S. aureus strain Newman is a human clinical isolate that stably maintains an agr⁺ phenotype, i.e. quorum controlled toxin secretion and the ability to cause animal disease (Duthie et al., 1952), whereas most other human clinical isolates rapidly lose the agr phenotype and with it the property of causing purulent infections in animals (Novick 2003).

A. Mutagenesis

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three. Transposable elements can also be used to “knock in” heterologous sequences.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another. In accordance with the present invention, mutations will be introduced into gram-positive bacteria such as S. aureus using a Himar 1 transposase.

Himar 1 is a “mariner,” one of a widespread and diverse family of animal transposons. Himar 1 is derived from Haematobia irritans. This transposase can reproduce transposition faithfully in an in vitro inter-plasmid transposition reaction. It binds to the inverted terminal repeat sequences of its cognate transposon and mediates S′ and 3′ cleavage of the element termini. It functions independent of species-specific host factors, which explains the broad distribution of mariners and why they are capable of horizontal transfer between species (Lampe et al., 1996).

B. Other Gram-Positive Bacteria

The present invention can be applied to other gram-positive bacteria as well. Gram-positive bacteria have a cell wall structure that contains peptidoglycan, in addition to polysaccharides and/or techoic acids. There are three general classes of gram-positive bacilli: Bacillacae, Micrococcaceae and Peptococcaceae. There are two major types of gram-positive cocci: Staphylococcous and Streptococcus.

1. Bacillacae

The Bacillacae are non-acid fast straight rod-shaped bacteria that are capable of forming spores. Some are motile, by virtue of peritrichous flagellae. They include aerobes, facultative anaerobes, and strict anaerobes and are generally non-halophylic with a wide growth range depending on the group. The biochemical actions seen in this family include fermentation and proteolytic activity. Some species can fix nitrogen, and a number of them also produce specific toxins. Bacillus is the type genus. The two main genera are Bacillus and Clostridium.

Bacillus: These are peritrichoulsly flagellated, form ellipsoidal or spherical, endospores, which may or may not swell the sporangium. They are aerobic to facultatively anaerobic and generally catalase-positive. There are currently very many species in this genus, including, B. anthracis, B. azotoformans, B. cereus, B. coagulans, B. israelensis, B. larvae, B. mycoides, B. polymyxa, B. pumilis, B. stearothormophillus, B. subtilis, B. thuringiensis and B. validus. Other genera include Sporolactobacilli, Sporocarcina, Filibacter and Caryophanum.

Clostridium: An inability to grow in air is the characteristic of this group, although some may tolerate it. Clostridium has many species, including psychrophilic, mesophilic or thermophilic members. They are generally Gram-positive with peritrichous flagellation, and can degrade organic materials. Acids, such as butyric acid, are a frequent product of fermentation by these organisms. They can form ellipsoidal or spherical, endospores. They tend to be grouped into saccharolytic or proteolytic species, but some are both.

Saccharolytic species include Cl. aerotolerans, Cl. aurantibutyricum, Cl. beijerinckii, Cl. botulinum B,E,F*, Cl. butyricum, Cl. chauvoei, Cl. difficile, Cl. intestinale, Cl. novyi A, Cl. pateurianum, Cl. saccharolyticum, Cl. septicum, Cl. thermoaceticum, and Cl. thermosaccharolyticum. The proteolytic species include: Cl. argeninense, Cl. ghoni, Cl. limosum, Cl. putrefaciens, Cl. subterminale and Cl. tetani. The proteolytic and saccharolytic species include Cl. acetobutylicum, Cl. bifermenans, Cl. botulinum A, B, F (prot)*, Cl. botulinum C,D*, Cl. cadaveris, Cl. haemolyticum, Cl. novyi B, C,* Cl. perfringens, Cl. putrefaciens, Cl. sordelli and Cl. sporogenes. The specialist species include Cl. acidiurici, Cl. irregularis, Cl. kluyveri, Cl. oxalicum, Cl. propionicum, Cl. sticklandii and Cl. villosum. Cl. botulinum is subdivided into a number of types according to the serological specificities of the toxins produced. These specificities are based on neutralisation studies (*other Clostridium species that produce botulinum toxins).

2. Micrococcoceae

These are gram-positive, non-sporing non-acid fast cocci They generally occur in tetrads (groups of four) or clusters and are not motile. They are generally aerobes and produce catalase. Carotenoid pigments are produced by most species. Most will grow on bacteriological media such as nutrient agar at 37° C. except some psychrotropic and halophyllic species which require cooler temperatures or 5% NaCl. There are only two genera in this family; Arthrobacter and Micrococcus. The genera Staphylococcus and Planococcus which were once considered part of this family are now excluded, due to marked differences in DNA base composition, cell wall, fatty acids and other compositions.

Arthrobacter: This group includes two species—the A. globiformis/A. citreus group and the A. nicotianae group—based on differences in cell wall structure. They are important soil organisms and the numerically largest number of isolates on aerobic primary isolation media.

Micrococcus: This genus has nine species, of which M. luteus, M. lylae, M. roseus are most studied. M. agilis differs from other species in being motile, psychrophilic and producing beta-galactosidase. M. kristinae can ferment glucose anaerobically and M. halobius requires at least 5% NaCl for growth.

3. Peptococcocaceae

These are gram-positive, non-acid fast cocci that do not form spores. They are strict anaerobes and are generally non-halophylic with a wide growth range. Many metabolize peptones and amino acids, and some require fermentable carbohydrates. The family includes fermenters, indole producers, nitrate reducers, urease producers, and those that express coagulase or catalase activity. Peptococcus is the type genus, and other genera include Peptostreptococcus, Sarcina and Coprococcus.

Peptococcus: Including only one species, P. niger, the cocci are arranged as diplococci, irregular clumps or regular clusters. They are anaerobic and n-caproic acid and butyric acid are the main metabolic products.

Peptostreptococcus: This genus has three groups. Ps. anaerobius is the only representative of one group. It is similar to P. niger, but grows confluently on commercial media and forms short chains. The other two groups form clumps and clusters, while some species form diplococci. Each group includes about five-six species, which are differentiated on their morphology and biochemical reactions including fermentation, reduction of nitrate, production of indole, urease, coagulase or catalase, etc.

Other genera include Ruminococcus, Sarcina, and Coprococcus.

4. Streptococci

Streptococci are spheroidal bacteria in the family Streptococcaceae. The term streptococcus (“twisted berry”) refers to the bacteria's characteristic grouping in chains resembling a string of beads. Streptococci can also be classified by the type of carbohydrate contained in the cell wall, a system called the Lancefield classification.

Streptococcus pyogenes: Group A S. pyogenes species of streptococci cause rheumatic fever, scarlet fever, erysipelas, strep throat, tonsillitis, and other upper respiratory infections. In common practice Streptococcus group A is usually found in samples from the throat, nasopharanyx, or in sputum in which a plethora of unimportant normal flora are also present. Its characteristic type of hemolysis is key for detection. If there is an area of clear or “beta,” hemolysis, the possibility of the presence of group A organisms exists, although the same type of hemolysis pattern may also be exhibited by some other organisms such as some gram negative rods which may also be present. Immunofluorescence can be used to confirm the group A diagnosis.

Streptococcus agalactiae: Group B is the most common cause of sepsis (blood infection) and meningitis (infection of the fluid and lining surrounding the brain) in newborns. Group B Streptococcus (GBS) is a frequent cause of newborn pneumonia and is more common than other, better known, newborn problems such as rubella, congenital syphilis, and spina bifida. One of every 20 babies with GBS disease dies from infection. Babies that survive, particularly those who have meningitis, may have long-term problems, such as hearing or vision loss or learning disabilities. In pregnant women, GBS can cause bladder infections, womb infections (amnionitis, endometritis), and stillbirth. Among men and among women who are not pregnant, the most common diseases caused by GBS are blood infections, skin or soft tissue infections, and pneumonia. Approximately 20% of men and nonpregnant women with GBS disease die of the disease. Adults can carry GBS in the bowel, vagina, bladder, or throat. People who carry GBS typically do so transiently.

GBS disease is diagnosed when the bacterium is grown from cultures of sterile body fluids, such as blood or spinal fluid. Cultures take a few days to complete. GBS infections in both newborns and adults are usually treated with i.v. antibiotics.

Streptococcus mutans is part of the normal mouth flora and is responsible for cavities. A vaccine is thought possible for Streptococcus mutans for the prevention of cavities.

5. Staphylococcus

Staphylococcus epidermidis (coagulase-negative) appears as white colonies on sheep blood agar plates. Like S. aureus, S. epidermidis is a normal inhabitant of the skin, but it is considered the lesser of the two in terms of virulence. Infections by S. epidermidis, S. haemolyticus, and S. hominis, the latter two also being coagulase negative, are associated with infections of intravascular devices (prosthetic heart valves and intra-arterial or intravenous lines), shunts and prosthetic joints, wound infections, osteomyelitis associated with foreign bodies, and endocarditis. Another clinically significant species is S. saprophyticus.

II. Nucleic Acids and Transformation

In accordance with the present invention, it may be desirable to manipulate transposon sequences to carry additional elements, which are described below. Further, transposon sequences will be transferred into bacterial host cells using the techniques described. Any nucleic acid sequence provided herein can be implemented with respect to any other embodiment. For example, a nucleic acid element may be contained in a plasmid, vector, host cell, or bacteria.

A. Vectors

The term “vector” is used to refer to a nucleic acid molecule into which a nucleic acid sequence can be inserted for introduction into a cell where it can be replicated and/or expressed. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules may then be translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

The plasmids pFA545 (SEQ IUD NO:1) and pBursa (SEQ ID NO:2) are described herein. Any of the functional genes and nucleic acid regions of these plasmids may be subcloned into other plasmids or vectors.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment.

In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous, and will be selected for activity in gram-positive bacteria.

2. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Origins of Replication

In order to propagate a vector in a host cell, it may contain one or more origins of replication sites (often termed “ori”), which is a specific nucleic acid sequence at which replication is initiated. The origin of replication may optionally be active or non-active at specific temperatures, i.e., temperature sensitive.

5. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ fluorescent or chemilluminescent markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Host Cell

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors or viruses (which does not qualify as a vector if it expresses no exogenous polypeptides). A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including bacteria cells, yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors.

Many host cells from various cell types and organisms are available and would be known to one of skill in the art. Similarly, a viral vector may be used in conjunction with either a eukaryotic or prokaryotic host cell, particularly one that is permissive for replication or expression of the vector.

C. Transformation

In accordance with the present invention, nucleic acid sequences are transferred into bacterial cells using standard methodologies known to those of ordinary skill in the art. In some instances, cells can be rendered competent for transformation by chemical treatment with CaCl (Sambrook et al. 1989). Some other methods are described below.

1. Electroporation

In certain embodiments of the present invention, the expression construct is introduced into the cell via electroporation. Electroporation involves the exposure of a suspension of cells and DNA to a high-voltage electric discharge. Electroporation works well with bacteria.

2. Calcium Phosphate or DEAE-Dextran

In other embodiments of the present invention, the expression construct is introduced to the cells using calcium phosphate precipitation. In another embodiment, the expression construct is delivered into the cell using DEAE-dextran followed by polyethylene glycol.

3. Particle Bombardment

Another embodiment of the invention for transferring a naked DNA expression construct into cells involves particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

4. Direct Microinjection or Sonication Loading

Further embodiments of the present invention include the introduction of the expression construct by direct microinjection or sonication loading. Direct microinjection has been used to introduce nucleic acid constructs into Xenopus oocytes (Harland and Weintraub, 1985), and LTK- fibroblasts have been transfected with the thymidine kinase gene by sonication loading (Fechheimer et al., 1987).

C. Markers

In certain aspects of the present invention, cells containing transposon are identified with specific markers. Such markers confer on their recombinant hosts a readily detectable phenotype that emerges only under conditions where the transposon has integrated into the genome of the host. Generally reporter genes encode a polypeptide not otherwise produced by the host cell which is detectable by analysis of the cell culture, e.g., by drug-resistance, or fluorometric or spectrophotometric analysis of the cell culture.

1. Screening Markers

Exemplary enzymes include esterases, phosphatases, proteases (tissue plasminogen activator or urokinase) and other enzymes capable of being detected by their activity, as will be known to those skilled in the art. Contemplated for use in the present invention is green fluorescent protein (GFP) as a marker for transgene expression (Chalfie et al., 1994). The use of GFP does not need exogenously added substrates, only irradiation by near UV or blue light, and thus has significant potential for use in monitoring gene expression in living cells.

Other particular examples are the enzymes firefly and bacterial luciferase, and the bacterial enzymes β-galactosidase and β-glucuronidase. Other marker genes within this class are well known to those of skill in the art, and are suitable for use in the present invention.

2. Selection

Another class of reporter genes which confer detectable characteristics on a host cell are those which encode polypeptides, generally enzymes, which render their transformants resistant against toxins. Examples of this class of reporter genes are the neo gene (Colberre-Garapin et al., 1981) which protects host cells against toxic levels of the antibiotic G418, the gene conferring streptomycin resistance (U.S. Pat. No. 4,430,434), the gene conferring hygromycin B resistance (Santerre et al., 1984; U.S. Pat. Nos. 4,727,028, 4,960,704 and 4,559,302), a gene encoding dihydrofolate reductase, which confers resistance to methotrexate (Alt et al., 1978), the enzyme HPRT, along with many others well known in the art (Kaufman, 1990). Chloramphenicol acetyltransferase (CAT) confer resistance to chloramphenicol, and the beta-lactamase gene confers ampicillin resistance.

III. Screening Methods

In accordance with the present invention, following transposon mutagenesis, one will select appropriate transformed strains using a variety of criteria. At a first level of selection, one will simply wish to identify those bacterial host cells that have taken up the transposon. At a second level, one will look for particular functional attributes of the transformants, presumably based on the site of integration of the transposon and the effect caused thereby. And at a third level, it may be desirable to identify the precise nature and location of the integration, optionally for the purpose of identifying bacterial genes or assigning function thereof. Various methods for achieving each of these selections are described below.

A. Selection Based on Antibiotic Resistance

In one embodiment, the transposon will contain a selectable marker. A selectable marker is an element, usually a polypeptide, that permits ready identification and selection of a transformant. A classic and often used selectable marker is a gene encoding a protein that confers resistance to an antibiotic. Antibiotics useful in selection procedures are well known to those of skill in the art and include chloramphenicol, ampicillin, hygromycin B, puromycin, zeocin, G418 and others.

Methods for antibiotic selection are well known to those of skill in the art. Appropriate concentrations of antibiotics are well known based on the desired purpose. Bacterial cells are cultured with antibiotics under other conditions otherwise suitable for growth of host cells. Selection may also involve using increasing concentrations of antibiotic with successive rounds of more rigorous selection. Selection may be in broth culture or plated on agar, or successive combinations of both.

B. Selection Based on Fluorescence

In another embodiment, transformants may be selected on the basis of expression of a fluorescent or a luminescent marker. The marker may be red fluorescent protein, green fluorescent protein, cyan fluorescent protein, or variants thereof. Luminescent markers include luciferase.

In this aspect, the present invention may take advantage of fluorescence-activated cell sorting (FACS). This technique utilizes a machine that can rapidly separate cells in a suspension on the basis of size and the color of their fluorescence. Generally, a cell suspension containing cells labeled with a fluorescent protein is directed into a thin stream so that all the cells pass in single file. This stream emerges from a nozzle vibrating at some 40,000 cycles per second which breaks the stream into 40,000 discrete droplets each second. Some of these may contain a cell. A laser beam is directed at the stream just before it breaks up into droplets. As each labeled cell passes through the beam, its resulting fluorescence is detected by a photocell. If the signals from the two detectors meet either of the criteria set for fluorescence and size, an electrical charge (+ or −) is given to the stream. The droplets retain this charge as they pass between a pair of charged metal plates. Positively-charged drops are attracted to the negatively-charged plate and vice versa. Uncharged droplets (those that contain no cell or a cell that fails to meet the desired criteria of fluorescence and size) pass straight into a third container and are later discarded. This apparatus can sort as many as 300,000 cells per minute.

C. Selection Based on Pathogenicity

In another aspect, the present invention will attempt to identify functional differences between bacterial mutants and their parental strains. The functional differences maybe any of a variety of different traits including growth, doubling time, nutrient dependence, drug susceptibility or pathogenicity.

A particular pathogenicity assay involves the testing of bacterial strains against C. elegans. When incubated on agar plates with either L4 larvae or adult worms, staphylococci enter the worm oral cavity, pass the food grinder and multiply in the worm's digestive tract. Colonization with S. aureus causes distension of the digestive tract and ultimately killing of infected worms over a period of 2-7 days (Sifri et al., 2003). Using this assay, Sifri and colleagues demonstrated that several known S. aureus virulence factors are also required for the killing of C. elegans (Sifri et al., 2003).

Another pathogenicity assay involves a murine model. S. aureus strains are inoculated intravenously into mice, which are euthanized four days after infection and assessed for gross pathology in the organs (Mazmanian et al., 2002).

D. Promoter Identification Assays

In another embodiment, the present invention will rely on the use of transposons that contain segments encoding marker genes, but lacking the regulatory sequences needed to effect transcription. However, if the transposon integrates near a promoter sequence, the adjacent promoter may drive expression of the otherwise promoter-less marker gene, thereby permitting identification/selection as described elsewhere in the document. It will generally be desired that the promoter-less marker be located near one terminus of the transposon, and that the 5′ end of the marker be closest to the nearest transposon terminus.

E. Identifying Insertion Sites

Finally, in some embodiments, it may be desirable to identify where a particular transposon has inserted. This can be performed at the level of rough genetic mapping using RFLP-type procedures (restriction followed by size separation of genomic fragments), or by sequencing. The latter will permit the precise identification of sequences that have been interrupted, and in some cases, attribute previously undescribed functions to a gene of interest. Sequencing may be effected in a variety of fashions using transposon sequences to prime synthesis into adjacent genomic sequences.

IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

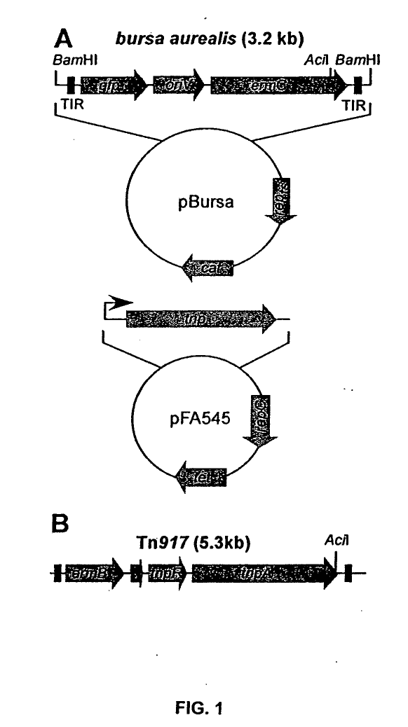

Transposon mutagenesis. S. aureus strain Newman was sequentially transformed with pFA545 and pBursa (FIG. 1) (GenBank accession nos. AY672108 and AY672109, respectively, which are hereby incorporated by reference). The resulting transformants were spread on tryptic soy agar containing 10 μg ml⁻¹ erythromycin (TSAₑᵣₘ) and incubated at 30° C. overnight. Isolated single colonies were suspended in 200 μl sterile water, spread on TSAₑᵣₘ, and then incubated at 43° C. for two days to select against the plasmids. Isolated single colonies were inoculated in tryptic soy broth containing 10 μg/ml erythromycin (TSBₑᵣₘ) and incubated at 43° C. overnight. Cells were collected by centrifugation, suspended in 100 μl TSM buffer (50 mM Tris-HCl, pH 7.5, 0.5 M sucrose, 10 mM MgCl₂) and then treated with lysostaphin (0.1 mg/ml final concentration) (Schindler et al., 1964) at 37° C. for 15 minutes. After collecting protoplasts by centrifugation, chromosomal DNA was purified with the Wizard Genomic DNA purification kit (Promega) according to the manufacturer's recommendation. AciI (NEB) was used to digest chromosomal DNA in 10 μl reaction volumes for 1-3 hours at 37° C. The digested DNA was ligated with T4 ligase in 100 μl reaction volume for 5-16 hours at room temperature and the resulting DNA was purified with QlAquick PCR purification kit (QIAGEN). DNA fragments carrying transposon/chromosome junction sequences were amplified by PCR with the primers: Martn-F (TTT ATG GTA CCA TTT CAT TTT CCT GCT TTT TC) and Martn-ermR (AAA CTG ATT TTT AGT AAA CAG TTG ACG ATA TTC). The annealing temperature was 63° C. and the DNA was amplified for 3 minutes with 40 cycles. After 0.8% agarose gel electrophoresis, PCR products were directly sequenced with the Martn-F primer at the sequencing facility of the University of Chicago Cancer Research Center.

Nematode killing assay. The C. elegans infection assay was carried out as previously reported (Sifri et al., 2003) with the following modifications. To synchronize the growth of worms, eggs were collected via the hypochlorite method (Lewis et al., 1995). The collected eggs were incubated in M9 buffer for 16-22 hours at room temperature and worms that had been arrested at the L1 larvae stage were plated on nutrient growth medium (NGM) agar that had been inoculated with E. coli OP50. Agar plates were incubated at 15° C. for 72 hours to generate L4 larvae or for 84-88 hours to generate adult worms. For initial screening procedures (screens 1-3, Table 1), 30-50 adult worms were used, while 50-100 L4 larvae or adult worms were infected for the final screening assay (screen 4, Table 1—located after Examples) after transduction of transposon insertions into the wild-type strain background. Worms were transferred with a platinum wire and autoclaved Elmer's Glue™ to TSA plates that had been inoculated with staphylococci, incubated at 25° C. for 48 hours, and live worms counted under a stereomicroscope (Nicon SMZ645). The nematode strain N2 and E. coli OP50 used in this work were provided by the Caenorhabditis Genetics Center, which is funded by the NIH National Center for Research Resources (NCRR).

Murine intra-venous infection and abscess formation. S. aureus transposon mutants were grown at 37° C. overnight in TSBₑᵣₘ, diluted 100 fold in fresh media and incubated at 37° C. for 2.5 hours until the cultures reached OD₆₀₀ 1.0. Bacteria were collected by centrifugation, washed and suspended in phosphate buffered saline (PBS). Viable staphylococci were enumerated by colony formation on TSA plates to quantify the infectious dose. One hundred microliter of bacterial suspension (˜1.3×10⁷ colony forming units) was administered intra-venously via retro-orbital injection into each of ten six- to eight-week old BALB/c mice. Four days after the injection, the mice were euthanized by CO₂ asphyxiation and the kidneys, spleen and liver were removed. The organs were homogenized in 1 ml of PBS and dilutions of the homogenates were plated on TSA;

Statistical analysis. Chi-square analysis was used to examine the statistical significance of worm killing data. Student's t-test was performed for statistical analysis of murine infection data, using the statistical software Analyse-it™.

TABLE 1

| Mutant | | Screen | | | |
|---|---|---|---|---|---|
| Number | ORF | 1 | Screen 2 | Screen 3 | Screen 4 |
| ΦNΞ 09697 | SAV0006 | 105 | | | |
| ΦNΞ 01964 | SAV0007 | 100 | | | |
| ΦNΞ 02761 | SAV0008 | 119 | | | |
| ΦNΞ 04440 | SAV0008 | 94 | | | |
| ΦNΞ 06169 | SAV0010 | 169 | | | |
| ΦNΞ 13102 | SAV0013 | 86 | 100 | | |
| ΦNΞ 04509 | SAV0013 | 118 | | | |
| ΦNΞ 03581 | SAV0017 | 105 | | | |
| ΦNΞ 02689 | SAV0017 | 125 | | | |
| ΦNΞ 11611 | SAV0022 | 67 | 79 | | 96 |
| ΦNΞ 04297 | SAV0023 | 84 | | | 100 |
| ΦNΞ 00158 | SAV0024 | 90 | | | |
| ΦNΞ 00007 | SAV0031 | 100 | | | |
| ΦNΞ 06084 | SAV0071 | 119 | | | |
| ΦNΞ 02921 | SAV0082 | 113 | | | |
| ΦNΞ 01632 | SAV0082 | 89 | | | |
| ΦNΞ 02710 | SAV0084 | 125 | | | |
| ΦNΞ 03605 | SAV0084 | 65 | | | |
| ΦNΞ 11469 | SAV0085 | 104 | | | |
| ΦNΞ 07569 | SAV0086 | 100 | | | |
| ΦNΞ 02897 | SAV0087 | 100 | 90 | 80 | |
| ΦNΞ 01747 | SAV0088 | 100 | | | |
| ΦNΞ 04416 | SAV0089 | 65 | 100 | | |
| ΦNΞ 04522 | SAV0093 | 118 | | | |
| ΦNΞ 08742 | SAV0094 | 86 | 104 | | |
| ΦNΞ 01578 | SAV0094 | 104 | | | |
| ΦNΞ 04331 | SAV0096 | 84 | | | |
| ΦNΞ 04701 | SAV0100 | 100 | | | |
| ΦNΞ 03975 | SAV0100 | 90 | | | |
| ΦNΞ 01752 | SAV0101 | 42 | 100 | | |
| ΦNΞ 01586 | SAV0101 | 79 | | | |
| ΦNΞ 12459 | SAV0102 | 90 | | | |
| ΦNΞ 04675 | SAV0103 | 100 | | | |
| ΦNΞ 03889 | SAV0104 | 95 | | | |
| ΦNΞ 09435 | SAV0105 | 91 | | | |
| ΦNΞ 01885 | SAV0106 | 95 | | | |
| ΦNΞ 05474 | SAV0107 | 105 | | | |
| ΦNΞ 05622 | SAV0107 | 105 | | | |
| ΦNΞ 04993 | SAV0108 | 85 | 75 | 56 | 100 |
| ΦNΞ 02760 | SAV0109 | 119 | | | |
| ΦNΞ 02284 | SAV0110 | 79 | 100 | | |
| ΦNΞ 03637 | SAV0111 | 100 | | | |
| ΦNΞ 11086 | SAV0112 | 82 | 50.9 | | |
| ΦNΞ 00251 | SAV0113 | 75 | | | |
| ΦNΞ 05329 | SAV0113 | 100 | | | |
| ΦNΞ 12605 | SAV0114 | 73 | 93 | | |
| ΦNΞ 04101 | SAV0115 | 65 | 75 | 92 | 100 |
| ΦNΞ 05598 | SAV0116 | 105 | | | |
| ΦNΞ 09972 | SAV0117 | 105 | | | |
| ΦNΞ 00750 | SAV0118 | 80 | | | |
| ΦNΞ 04431 | SAV0119 | 100 | | | |
| ΦNΞ 04528 | SAV0120 | 118 | | | |
| ΦNΞ 03981 | SAV0121 | 95 | | | |
| ΦNΞ 00568 | SAV0121 | 85 | | | |
| ΦNΞ 06601 | SAV0122 | 166 | | | |
| ΦNΞ 00693 | SAV0123 | 85 | | | |
| ΦNΞ 02682 | SAV0124 | 125 | | | |
| ΦNΞ 00114 | SAV0125 | 95 | | | |
| ΦNΞ 02811 | SAV0126 | 119 | | | |
| ΦNΞ 00797 | SAV0128 | 100 | | | |
| ΦNΞ 02789 | SAV0129 | 125 | | | |
| ΦNΞ 11103 | SAV0130 | 86 | 49.1 | | |
| ΦNΞ 01699 | SAV0130 | 105 | | | |
| ΦNΞ 00235 | SAV0131 | 95 | | | |
| ΦNΞ 00540 | SAV0132 | 95 | | | |
| ΦNΞ 02728 | SAV0133 | 113 | | | |
| ΦNΞ 01938 | SAV0134 | 105 | | | |
| ΦNΞ 00707 | SAV0134 | 70 | | | |
| ΦNΞ 00256 | SAV0135 | 95 | | | |
| ΦNΞ 04115 | SAV0136 | 89 | | | |
| ΦNΞ 04163 | SAV0137 | 105 | | | |
| ΦNΞ 04768 | SAV0139 | 100 | | | |
| ΦNΞ 04556 | SAV0140 | 118 | | | |
| ΦNΞ 04840 | SAV0141 | 100 | | | |
| ΦNΞ 03501 | SAV0142 | 105 | | | |
| ΦNΞ 04512 | SAV0143 | 118 | | | |
| ΦNΞ 00009 | SAV0144 | 106 | | | |
| ΦNΞ 06375 | SAV0145 | 153 | | | |
| ΦNΞ 06399 | SAV0147* | 169 | | | |
| ΦNΞ 01814 | SAV0148 | 100 | | | |
| ΦNΞ 10032 | SAV0149 | 105 | | | |
| ΦNΞ 05254 | SAV0150 | 100 | | | |
| ΦNΞ 05138 | SAV0151 | 100 | | | |
| ΦNΞ 02051 | SAV0152 | 105 | | | |
| ΦNΞ 11309 | SAV0153 | 118 | | | |
| ΦNΞ 05448 | SAV0154 | 53 | 10 | 49 | 52 |
| ΦNΞ 05573 | SAV0155 | 100 | | | |
| ΦNΞ 11624 | SAV0156 | 63 | 62 | | |
| ΦNΞ 04851 | SAV0157 | 40 | 55 | 71 | 43 |
| ΦNΞ 03492 | SAV0158 | 68 | 55 | 62 | 87 |
| ΦNΞ 04856 | SAV0159 | 75 | 95 | | |
| ΦNΞ 00618 | SAV0160 | 70 | | | |
| ΦNΞ 01957 | SAV0161 | 26 | 75 | 25 | |
| ΦNΞ 01866 | SAV0162 | 89 | | | |
| ΦNΞ 05283 | SAV0163 | 10 | 35 | 67 | 75 |
| ΦNΞ 04700 | SAV0164 | 100 | | | |
| ΦNΞ 11092 | SAV0165 | 132 | | | |
| ΦNΞ 04350 | SAV0166 | 94 | | | |
| ΦNΞ 02715 | SAV0167 | 125 | | | |
| ΦNΞ 03273 | SAV0168 | 63 | 95 | | |
| ΦNΞ 05237 | SAV0171 | 95 | | | |
| ΦNΞ 02635 | SAV0172 | 105 | | | |
| ΦNΞ 03639 | SAV0173 | 112 | | | |
| ΦNΞ 05563 | SAV0174 | 105 | | | |
| ΦNΞ 00698 | SAV0175 | 100 | | | |
| ΦNΞ 02233 | SAV0176 | 74 | 100 | | |
| ΦNΞ 07526 | SAV0177 | 70 | 93 | | |
| ΦNΞ 00016 | SAV0179 | 75 | | | |
| ΦNΞ 03096 | SAV0180 | 56 | 75 | 92 | 100 |
| ΦNΞ 02265 | SAV0182 | 89 | | | |
| ΦNΞ 12640 | SAV0183 | 67 | 96 | | |
| ΦNΞ 02803 | SAV0184 | 125 | | | |
| ΦNΞ 05074 | SAV0185 | 70 | 65 | 36 | 97 |
| ΦNΞ 10656 | SAV0186 | 93 | | | |
| ΦNΞ 08784 | SAV0187 | 63 | 100 | | |
| ΦNΞ 03140 | SAV0188 | 75 | 70 | 88 | 93 |
| ΦNΞ 01753 | SAV0189 | 105 | | | |
| ΦNΞ 04337 | SAV0190 | 47 | 95 | | |
| ΦNΞ 00006 | SAV0192 | 94 | | | |
| ΦNΞ 02135 | SAV0193 | 105 | | | |
| ΦNΞ 02429 | SAV0195 | 105 | | | |
| ΦNΞ 00710 | SAV0195 | 65 | | | |
| ΦNΞ 05126 | SAV0195 | 100 | | | |
| ΦNΞ 11739 | SAV0205 | 105 | | | |
| ΦNΞ 10862 | SAV0206 | 100 | | | |
| ΦNΞ 04004 | SAV0207 | 85 | | | |
| ΦNΞ 12525 | SAV0208 | 83 | 104 | | |
| ΦNΞ 03761 | SAV0209 | 76 | | | |
| ΦNΞ 13516 | SAV0210 | 96 | | | |
| ΦNΞ 10277 | SAV0211* | 105 | | | |
| ΦNΞ 04162 | SAV0212 | 63 | | | |
| ΦNΞ 06383 | SAV0213 | 131 | | | |
| ΦNΞ 03834 | SAV0214 | 85 | | | |
| ΦNΞ 13547 | SAV0215 | 76 | 107 | | |
| ΦNΞ 02950 | SAV0216 | 81 | 95 | | |
| ΦNΞ 00051 | SAV0216 | 88 | | | |
| ΦNΞ 12364 | SAV0217 | 63 | 100 | | |
| ΦNΞ 04534 | SAV0218 | 118 | | | |
| ΦNΞ 03836 | SAV0219 | 75 | 95 | | |
| ΦNΞ 01499 | SAV0219 | 85 | | | |
| ΦNΞ 04364 | SAV0220 | 47 | 95 | | |
| ΦNΞ 00616 | SAV0222 | 75 | | | |
| ΦNΞ 00716 | SAV0222 | 85 | | | |
| ΦNΞ 00452 | SAV0223 | 45 | 100 | | |
| ΦNΞ 02933 | SAV0224 | 88 | 90 | | |
| ΦNΞ 00232 | SAV0225 | 100 | | | |
| ΦNΞ 05431 | SAV0226 | 105 | | | |
| ΦNΞ 04690 | SAV0228 | 100 | | | |
| ΦNΞ 14033 | SAV0229 | 93 | | | |
| ΦNΞ 00492 | SAV0230 | 95 | | | |
| ΦNΞ 04061 | SAV0231 | 112 | | | |
| ΦNΞ 02733 | SAV0231 | 119 | | | |
| ΦNΞ 00054 | SAV0232 | 119 | | | |
| ΦNΞ 03997 | SAV0233 | 100 | | | |
| ΦNΞ 04666 | SAV0234 | 100 | | | |
| ΦNΞ 00796 | SAV0235 | 85 | | | |
| ΦNΞ 03764 | SAV0235 | 94 | | | |
| ΦNΞ 00015 | SAV0236 | 106 | | | |
| ΦNΞ 01810 | SAV0237 | 100 | | | |
| ΦNΞ 11473 | SAV0238 | 46 | 83 | | |
| ΦNΞ 04861 | SAV0240 | 100 | | | |
| ΦNΞ 02831 | SAV0241 | 125 | | | |
| ΦNΞ 10824 | SAV0242 | 64 | 86 | | 100 |
| ΦNΞ 02365 | SAV0243 | 95 | | | |
| ΦNΞ 12294 | SAV0244 | 87 | | | |
| ΦNΞ 03950 | SAV0245 | 90 | | | |
| ΦNΞ 05635 | SAV0246 | 105 | | | |
| ΦNΞ 05298 | SAV0248 | 55 | 50 | 25 | 95 |
| ΦNΞ 05235 | SAV0250 | 95 | | | |
| ΦNΞ 01934 | SAV0251 | 95 | | | |
| ΦNΞ 05236 | SAV0252 | 100 | | | |
| ΦNΞ 03224 | SAV0258 | 63 | 50 | 85 | 85 |
| ΦNΞ 04126 | SAV0258 | 42 | 60 | 75 | 97 |
| ΦNΞ 00118 | SAV0259 | 95 | | | |
| ΦNΞ 02846 | SAV0260 | 113 | | | |
| ΦNΞ 00645 | SAV0260 | 60 | | | |
| ΦNΞ 05796 | SAV0261 | 105 | | | |
| ΦNΞ 00684 | SAV0262 | 75 | | | |
| ΦNΞ 05502 | SAV0264 | 105 | | | |
| ΦNΞ 11126 | SAV0265 | 27 | 83 | | 98 |
| ΦNΞ 10852 | SAV0266 | 86 | | | |
| ΦNΞ 09554 | SAV0267 | 55 | 86 | | 94 |
| ΦNΞ 09840 | SAV0268 | 100 | 95 | | |
| ΦNΞ 05444 | SAV0270 | 105 | | | |
| ΦNΞ 03097 | SAV0271 | 106 | | | |
| ΦNΞ 07234 | SAV0272 | 97 | | | |
| ΦNΞ 12766 | SAV0273 | 47 | 86 | | |
| ΦNΞ 00275 | SAV0274 | 100 | | | |
| ΦNΞ 01870 | SAV0275 | 89 | | | |
| ΦNΞ 02987 | SAV0276 | 100 | | | |
| ΦNΞ 04665 | SAV0277 | 118 | | | |
| ΦNΞ 05550 | SAV0278 | 105 | | | |
| ΦNΞ 04368 | SAV0279 | 47 | 95 | | |
| ΦNΞ 03794 | SAV0280 | 106 | | | |
| ΦNΞ 02936 | SAV0281 | 100 | | | |
| ΦNΞ 09349 | SAV0283 | 123 | | | |
| ΦNΞ 03742 | SAV0284 | 106 | | | |
| ΦNΞ 11241 | SAV0285 | 132 | | | |
| ΦNΞ 02411 | SAV0286 | 100 | | | |
| ΦNΞ 03683 | SAV0287 | 24 | 85 | 93 | |
| ΦNΞ 04464 | SAV0287 | 106 | | | 63 |
| ΦNΞ 02755 | SAV0288 | 125 | | | |
| ΦNΞ 02832 | SAV0288 | 125 | | | |
| ΦNΞ 03493 | SAV0293 | 100 | | | |
| ΦNΞ 10978 | SAV0294 | 114 | | | |
| ΦNΞ 04048 | SAV0295 | 106 | | | |
| ΦNΞ 05120 | SAV0296 | 95 | | | |
| ΦNΞ 04317 | SAV0297 | 105 | | | |
| ΦNΞ 05433 | SAV0298 | 105 | | | |
| ΦNΞ 06103 | SAV0299 | 134 | | | |
| ΦNΞ 11844 | SAV0300 | 46 | 103 | | |
| ΦNΞ 10920 | SAV0302 | 32 | 100 | | |
| ΦNΞ 03864 | SAV0304 | 85 | | | |
| ΦNΞ 00605 | SAV0305 | 55 | 100 | | |
| ΦNΞ 03413 | SAV0306 | 79 | | | |
| ΦNΞ 03089 | SAV0307 | 119 | | | |
| ΦNΞ 00460 | SAV0308 | 70 | | | |
| ΦNΞ 13888 | SAV0309 | 90 | | | |
| ΦNΞ 04052 | SAV0310 | 102, 88 | | | |
| ΦNΞ 04086 | SAV0311 | 76 | 75 | 78 | 98 |
| ΦNΞ 06575 | SAV0312 | 175 | | | |
| ΦNΞ 05564 | SAV0313 | 105 | | | |
| ΦNΞ 03729 | SAV0314 | 71 | | | |
| ΦNΞ 09108 | SAV0315 | 72 | 105 | | |
| ΦNΞ 02871 | SAV0316 | 125 | | | |
| ΦNΞ 14683 | SAV0317 | 100 | | | |
| ΦNΞ 03752 | SAV0318 | 88 | | | |
| ΦNΞ 06115 | SAV0319 | 172 | | | |
| ΦNΞ 08795 | SAV0321 | 93 | | | |
| ΦNΞ 10723 | SAV0323 | 79 | | | |
| ΦNΞ 11496 | SAV0324 | 29 | 97 | | |
| ΦNΞ 05241 | SAV0325 | 100 | | | |
| ΦNΞ 04742 | SAV0326 | 100 | | | |
| ΦNΞ 02961 | SAV0327 | 94 | | | |
| ΦNΞ 04339 | SAV0327 | 37 | 85 | 58 | 100 |
| ΦNΞ 06073 | SAV0328 | 166 | | | |
| ΦNΞ 05517 | SAV0329 | 105 | | | |
| ΦNΞ 02277 | SAV0330 | 95 | | | |
| ΦNΞ 05320 | SAV0332 | 95 | | | |
| ΦNΞ 04148 | SAV0334 | 74 | | | |
| ΦNΞ 03498 | SAV0335 | 105 | | | |
| ΦNΞ 08713 | SAV0337 | 93 | | | |
| ΦNΞ 03817 | SAV0337 | 70 | | | |
| ΦNΞ 00413 | SAV0339 | 75 | | | |
| ΦNΞ 04056 | SAV0339 | 112 | | | |
| ΦNΞ 05037 | SAV0340 | 100 | | | |
| ΦNΞ 00424 | SAV0341 | 70 | | | |
| ΦNΞ 12477 | SAV0342 | 93 | | | |
| ΦNΞ 13656 | SAV0343 | 94 | | | |
| ΦNΞ 05333 | SAV0344 | 90 | | | |
| ΦNΞ 05694 | SAV0345 | 105 | | | |
| ΦNΞ 03851 | SAV0346 | 50 | 90 | | |
| ΦNΞ 10814 | SAV0348 | 90 | | | |
| ΦNΞ 11890 | SAV0350 | 50 | 93 | | |
| ΦNΞ 04881 | SAV0351 | 100 | | | |
| ΦNΞ 03635 | SAV0352 | 112 | | | |
| ΦNΞ 02127 | SAV0353 | 105 | | | |
| ΦNΞ 03134 | SAV0355 | 113 | | | |
| ΦNΞ 00735 | SAV0356 | 70 | | | |
| ΦNΞ 00204 | SAV0357 | 100 | | | |
| ΦNΞ 02989 | SAV0357 | 75 | 90 | | |
| ΦNΞ 08730 | SAV0358 | 100 | | | |
| ΦNΞ 04976 | SAV0359 | 100 | | | |
| ΦNΞ 03281 | SAV0360 | 79 | | | |
| ΦNΞ 11678 | SAV0361 | 92 | | | |
| ΦNΞ 06441 | SAV0363 | 184 | | | |
| ΦNΞ 01611 | SAV0368 | 43 | 85 | 98 | |
| ΦNΞ 13296 | SAV0370 | 102 | | | |
| ΦNΞ 02822 | SAV0372 | 106 | | | |
| ΦNΞ 07268 | SAV0375 | 70 | 100 | | |
| ΦNΞ 03854 | SAV0376 | 90 | | | |
| ΦNΞ 04161 | SAV0377 | 95 | | | |
| ΦNΞ 01991 | SAV0377 | 74 | 95 | | |
| ΦNΞ 11517 | SAV0378 | 71 | 100 | | |
| ΦNΞ 01746 | SAV0379 | 95 | | | |
| ΦNΞ 12691 | SAV0381 | 50 | 82 | | 96 |
| ΦNΞ 04519 | SAV0382 | 118 | | | |
| ΦNΞ 05489 | SAV0383 | 105 | | | |
| ΦNΞ 12236 | SAV0385 | 79 | 93 | | |
| ΦNΞ 09826 | SAV0386 | 105 | | | |
| ΦNΞ 09238 | SAV0388 | 94 | | | |
| ΦNΞ 04691 | SAV0389 | 100 | | | |
| ΦNΞ 02868 | SAV0390 | 125 | | | |
| ΦNΞ 02197 | SAV0391 | 105 | | | |
| ΦNΞ 10224 | SAV0416 | 105 | | | |
| ΦNΞ 11908 | SAV0417 | 100 | | | |
| ΦNΞ 00572 | SAV0420 | 60 | 100 | | |
| ΦNΞ 06130 | SAV0421 | 109 | 100 | | |
| ΦNΞ 04681 | SAV0424 | 100 | | | 102 |
| ΦNΞ 09645 | SAV0425 | 100 | 100 | | |
| ΦNΞ 03859 | SAV0426 | 75 | 95 | | |
| ΦNΞ 02183 | SAV0427 | 105 | | | |
| ΦNΞ 01645 | SAV0428 | 95 | | | |
| ΦNΞ 11723 | SAV0429 | 95 | | | |
| ΦNΞ 06340 | SAV0430 | 169 | | | |
| ΦNΞ 00606 | SAV0432 | 70 | | | |
| ΦNΞ 13910 | SAV0433 | 97 | | | |
| ΦNΞ 01618 | SAV0434 | 95 | | | |
| ΦNΞ 01996 | SAV0436 | 105 | | | |
| ΦNΞ 00586 | SAV0438 | 85 | | | |
| ΦNΞ 12927 | SAV0441 | 87 | | | |
| ΦNΞ 12268 | SAV0442 | 63 | 90 | | |
| ΦNΞ 04371 | SAV0444 | 100 | | | |
| ΦNΞ 00383 | SAV0445 | 100 | | | |
| ΦNΞ 05045 | SAV0446 | 80 | | | |
| ΦNΞ 05251 | SAV0449 | 100 | | | |
| ΦNΞ 04173 | SAV0450 | 47 | 90 | | |
| ΦNΞ 00537 | SAV0455 | 85 | | | |
| ΦNΞ 03741 | SAV0456 | 106 | | | |
| ΦNΞ 05316 | SAV0457 | 100 | | | |
| ΦNΞ 04955 | SAV0459 | 95 | | | |
| ΦNΞ 03554 | SAV0461 | 100 | | | |
| ΦNΞ 00099 | SAV0461 | 90 | | | |
| ΦNΞ 01861 | SAV0461 | 105 | | | |
| ΦNΞ 00841 | SAV0462 | 62 | | | |
| ΦNΞ 02295 | SAV0463 | 105 | | | |
| ΦNΞ 01857 | SAV0464 | 89 | | | |
| ΦNΞ 00339 | SAV0464 | 80 | | | |
| ΦNΞ 04405 | SAV0465 | 112 | | | |
| ΦNΞ 06582 | SAV0466* | 94 | | | |
| ΦNΞ 02115 | SAV0467 | 105 | | | |
| ΦNΞ 00464 | SAV0468 | 50 | 100 | | |
| ΦNΞ 04062 | SAV0469 | 112 | | | |
| ΦNΞ 03883 | SAV0470 | 80 | | | |
| ΦNΞ 12685 | SAV0471 | 67 | 96 | | |
| ΦNΞ 04151 | SAV0472 | 105 | | | |
| ΦNΞ 02133 | SAV0473 | 105 | | | |
| ΦNΞ 00072 | SAV0474 | 90 | | | |
| ΦNΞ 03871 | SAV0475 | 10 | 0 | | 10 |
| ΦNΞ 03557 | SAV0476 | 95 | | | |
| ΦNΞ 11167 | SAV0481 | 105 | | | |
| ΦNΞ 14049 | SAV0485 | 10 | 64 | | 96 |
| ΦNΞ 03926 | SAV0487 | 100 | | | |
| ΦNΞ 01612 | SAV0488 | 73 | 100 | | |
| ΦNΞ 04831 | SAV0489 | 90 | | | |
| ΦNΞ 13072 | SAV0489* | 102 | | | |
| ΦNΞ 05397 | SAV0490 | 105 | | | |
| ΦNΞ 03488 | SAV0492 | 105 | | | |
| ΦNΞ 05534 | SAV0493 | 105 | | | |
| ΦNΞ 08899 | SAV0495 | 33 | 107 | | 98 |
| ΦNΞ 09720 | SAV0499 | 105 | | | |
| ΦNΞ 09944 | SAV0501 | 105 | | | |
| ΦNΞ 02410 | SAV0501 | 105 | | | |
| ΦNΞ 09430 | SAV0503 | 32 | 71 | | 98 |
| ΦNΞ 04818 | SAV0511 | 60 | 65 | 33 | 75 |
| ΦNΞ 04925 | SAV0512 | 100 | | | |
| ΦNΞ 09310 | SAV0516 | 88 | 107 | | |
| ΦNΞ 04532 | SAV0518 | 118 | | | |
| ΦNΞ 03216 | SAV0518 | 89 | | | |
| ΦNΞ 10259 | SAV0520 | 105 | | | |
| ΦNΞ 04483 | SAV0521 | 100 | | | |
| ΦNΞ 00696 | SAV0522 | 60 | | | |
| ΦNΞ 02037 | SAV0523 | 105 | | | |
| ΦNΞ 03019 | SAV0525 | 113 | | | |
| ΦNΞ 00446 | SAV0525 | 60 | 100 | | |
| ΦNΞ 03634 | SAV0526 | 71 | | | |
| ΦNΞ 05910 | SAV0527 | 105 | | | |
| ΦNΞ 11689 | SAV0528 | 83 | | | |
| ΦNΞ 02860 | SAV0530 | 31 | 20 | | 97 |
| ΦNΞ 00708 | SAV0531 | 50 | 100 | | |
| ΦNΞ 11988 | SAV0532 | 58 | 93 | | |
| ΦNΞ 03612 | SAV0541 | 59 | 50 | 86 | 100 |
| ΦNΞ 03459 | SAV0549 | 105 | | | |
| ΦNΞ 03858 | SAV0550 | 100 | | | |
| ΦNΞ 01604 | SAV0551 | 110 | | | |
| ΦNΞ 04399 | SAV0552 | 71 | | | |
| ΦNΞ 02892 | SAV0552 | 119 | | | |
| ΦNΞ 00610 | SAV0553 | 70 | | | |
| ΦNΞ 08699 | SAV0555 | 90 | | | |
| ΦNΞ 03850 | SAV0556 | 75 | 100 | | |
| ΦNΞ 02743 | SAV0559 | 125 | | | |
| ΦNΞ 13068 | SAV0560 | 96 | | | |
| ΦNΞ 03932 | SAV0561 | 90 | | | |
| ΦNΞ 03506 | SAV0562 | 100 | | | |
| ΦNΞ 00583 | SAV0563 | 100 | | | 97 |
| ΦNΞ 03187 | SAV0564 | 100 | | | |
| ΦNΞ 09690 | SAV0567 | 105 | | | |
| ΦNΞ 04823 | SAV0568 | 95 | | | |
| ΦNΞ 11650 | SAV0569 | 83 | | | |
| ΦNΞ 12246 | SAV0570 | 88 | 90 | | |
| ΦNΞ 03062 | SAV0571 | 44 | 75 | 83 | 98 |
| ΦNΞ 03520 | SAV0572 | 84 | | | |
| ΦNΞ 03856 | SAV0573 | 80 | | | |
| ΦNΞ 04380 | SAV0573 | 65 | | | |
| ΦNΞ 09443 | SAV0574 | 95 | | | |
| ΦNΞ 04497 | SAV0575 | 118 | | | |
| ΦNΞ 05028 | SAV0576 | 95 | | | |
| ΦNΞ 06188 | SAV0577 | 131 | | | |
| ΦNΞ 03482 | SAV0580 | 89 | | | |
| ΦNΞ 10785 | SAV0581 | 86 | | | |
| ΦNΞ 06170 | SAV0583 | 159 | | | |
| ΦNΞ 05277 | SAV0584 | 75 | 95 | | |
| ΦNΞ 02439 | SAV0586 | 105 | | | |
| ΦNΞ 01545 | SAV0588 | 30 | 35 | | 5 |
| ΦNΞ 01995 | SAV0594 | 105 | | | |
| ΦNΞ 10439 | SAV0595 | 93 | | | |
| ΦNΞ 09988 | SAV0596 | 105 | | | |
| ΦNΞ 04142 | SAV0596 | 79 | | | |
| ΦNΞ 05200 | SAV0597 | 75 | 80 | 69 | 93 |
| ΦNΞ 02981 | SAV0598 | 125 | | | |
| ΦNΞ 06547 | SAV0598 | 88 | 100 | | |
| ΦNΞ 02070 | SAV0600 | 84 | | | |
| ΦNΞ 05263 | SAV0601 | 100 | | | |
| ΦNΞ 12002 | SAV0602 | 121 | | | |
| ΦNΞ 01599 | SAV0603 | 85 | | | |
| ΦNΞ 05238 | SAV0604 | 85 | | | |
| ΦNΞ 04470 | SAV0605 | 53 | 85 | 45 | 97 |
| ΦNΞ 00139 | SAV0609 | 95 | | | |
| ΦNΞ 04481 | SAV0610 | 53 | 80 | 51 | 98 |
| ΦNΞ 01637 | SAV0611 | 95 | | | |
| ΦNΞ 03143 | SAV0612 | 75 | 60 | 70 | 98 |
| ΦNΞ 03663 | SAV0615 | 59 | 100 | | |
| ΦNΞ 10017 | SAV0616 | 105 | | | |
| ΦNΞ 03709 | SAV0617 | 76 | | | |
| ΦNΞ 04096 | SAV0620 | 82 | 85 | 97 | |
| ΦNΞ 04837 | SAV0621 | 100 | | | |
| ΦNΞ 11326 | SAV0622 | 109 | | | |
| ΦNΞ 00286 | SAV0625 | 90 | | | |
| ΦNΞ 14384 | SAV0626 | 90 | | | |
| ΦNΞ 01786 | SAV0629 | 105 | | | |
| ΦNΞ 01485 | SAV0631 | 79 | | | |
| ΦNΞ 02717 | SAV0632 | 125 | | | |
| ΦNΞ 09193 | SAV0633 | 102 | | | |
| ΦNΞ 04913 | SAV0635 | 95 | | | |
| ΦNΞ 05382 | SAV0640 | 105 | | | |
| ΦNΞ 11552 | SAV0642 | 42 | 28 | | 100 |
| ΦNΞ 02038 | SAV0643 | 63 | 60 | 13 | 23 |
| ΦNΞ 12855 | SAV0644 | 93 | | | |
| ΦNΞ 03704 | SAV0645 | 59 | 85 | 97 | |
| ΦNΞ 03100 | SAV0646* | 69 | 75 | 83 | 98 |
| ΦNΞ 11205 | SAV0647 | 109 | | | |
| ΦNΞ 03749 | SAV0648 | 94 | | | |
| ΦNΞ 02667 | SAV0649 | 100 | | | |
| ΦNΞ 09237 | SAV0650 | 90 | | | |
| ΦNΞ 02257 | SAV0652 | 84 | | | |
| ΦNΞ 02489 | SAV0653 | 53 | 20 | 25 | |
| ΦNΞ 02849 | SAV0654 | 75 | | | |
| ΦNΞ 02091 | SAV0655 | 32 | 65 | 25 | |
| ΦNΞ 00623 | SAV0656 | 90 | | | |
| ΦNΞ 03803 | SAV0657 | 100 | | | |
| ΦNΞ 03813 | SAV0658 | 50 | 70 | 65 | 97 |
| ΦNΞ 04324 | SAV0659 | 89 | | | |
| ΦNΞ 03902 | SAV0660 | 50 | 75 | 63 | 98 |
| ΦNΞ 00587 | SAV0661 | 70 | | | |
| ΦNΞ 04764 | SAV0662 | 100 | | | |
| ΦNΞ 04345 | SAV0662 | 95 | | | |
| ΦNΞ 05491 | SAV0663 | 105 | | | |
| ΦNΞ 03029 | SAV0664 | 63 | 90 | | |
| ΦNΞ 09455 | SAV0665 | 59 | 107 | | 100 |
| ΦNΞ 05202 | SAV0667 | 95 | | | |
| ΦNΞ 12771 | SAV0668 | 90 | | | |
| ΦNΞ 04018 | SAV0669 | 80 | | | |
| ΦNΞ 03602 | SAV0672 | 118 | | | |
| ΦNΞ 02263 | SAV0672 | 105 | | | |
| ΦNΞ 01948 | SAV0673 | 89 | | | |
| ΦNΞ 01974 | SAV0674 | 100 | | | |
| ΦNΞ 10215 | SAV0675 | 105 | | | |
| ΦNΞ 05583 | SAV0675 | 95 | 95 | | |
| ΦNΞ 00507 | SAV0676 | 80 | | | |
| ΦNΞ 09638 | SAV0679 | 105 | | | |
| ΦNΞ 00785 | SAV0679 | 65 | | | |
| ΦNΞ 05086 | SAV0680 | 100 | | | |
| ΦNΞ 00836 | SAV0681 | 51 | 95 | | |
| ΦNΞ 00316 | SAV0684 | 100 | | | |
| ΦNΞ 00617 | SAV0685 | 75 | | | |
| ΦNΞ 03040 | SAV0686 | 88 | | | |
| ΦNΞ 04065 | SAV0687 | 112 | | | |
| ΦNΞ 10986 | SAV0690 | 109 | | | |
| ΦNΞ 12280 | SAV0691 | 58 | 100 | | |
| ΦNΞ 11360 | SAV0692 | 109 | | | |
| ΦNΞ 04797 | SAV0693 | 100 | | | |
| ΦNΞ 09710 | SAV0694 | 105 | | | |
| ΦNΞ 05267 | SAV0695 | 100 | | | |
| ΦNΞ 02147 | SAV0696 | 95 | | | |
| ΦNΞ 09918 | SAV0698 | 105 | | | |
| ΦNΞ 09835 | SAV0699 | 105 | | | |
| ΦNΞ 04930 | SAV0700 | 100 | | | |
| ΦNΞ 00659 | SAV0701 | 85 | | | |
| ΦNΞ 05064 | SAV0702 | 100 | | | |
| ΦNΞ 09837 | SAV0703 | 105 | | | |
| ΦNΞ 09725 | SAV0705 | 100 | 65 | 36 | 93 |
| ΦNΞ 01594 | SAV0706 | 98 | | | |
| ΦNΞ 02705 | SAV0707 | 125 | | | |
| ΦNΞ 09654 | SAV0708 | 105 | | | |
| ΦNΞ 01776 | SAV0709 | 16 | 90 | 53 | 78 |
| ΦNΞ 03711 | SAV0711 | 41 | 85 | 73 | 90 |
| ΦNΞ 11117 | SAV0713 | 91 | | | |
| ΦNΞ 02930 | SAV0714 | 119 | | | |
| ΦNΞ 00549 | SAV0715 | 85 | | | |
| ΦNΞ 05377 | SAV0717 | 100 | | | |
| ΦNΞ 04130 | SAV0718 | 95 | | | |
| ΦNΞ 04908 | SAV0720 | 100 | | | |
| ΦNΞ 09483 | SAV0721 | 27 | 100 | | 68 |
| ΦNΞ 09880 | SAV0722 | 105 | | | |
| ΦNΞ 05542 | SAV0723 | 105 | | | |
| ΦNΞ 02266 | SAV0724 | 100 | | | |
| ΦNΞ 05002 | SAV0725 | 90 | | | |
| ΦNΞ 00328 | SAV0726 | 95 | | | |
| ΦNΞ 04880 | SAV0727 | 85 | 85 | 71 | 97 |
| ΦNΞ 10547 | SAV0728 | 73 | | | |
| ΦNΞ 02913 | SAV0729 | 119 | | | |
| ΦNΞ 02988 | SAV0733 | 75 | 90 | | |
| ΦNΞ 02275 | SAV0733 | 63 | | | |
| ΦNΞ 02772 | SAV0734 | 125 | | | |
| ΦNΞ 08880 | SAV0735 | 92 | | | |
| ΦNΞ 00012 | SAV0736 | 100 | | | |
| ΦNΞ 01999 | SAV0737 | 74 | 100 | | |
| ΦNΞ 02004 | SAV0740 | 89 | | | |
| ΦNΞ 09297 | SAV0742 | 103 | | | |
| ΦNΞ 05140 | SAV0743 | 100 | | | |
| ΦNΞ 09363 | SAV0744 | 23 | 89 | | 100 |
| ΦNΞ 05370 | SAV0745 | 100 | | | |
| ΦNΞ 01804 | SAV0746 | 42 | 100 | | |
| ΦNΞ 13309 | SAV0748 | 96 | | | |
| ΦNΞ 12261 | SAV0749 | 83 | 103 | | |
| ΦNΞ 01492 | SAV0750 | 98 | | | |
| ΦNΞ 13522 | SAV0751 | 100 | | | |
| ΦNΞ 03632 | SAV0755 | 29 | 90 | | |
| ΦNΞ 00872 | SAV0755 | 92 | | | |
| ΦNΞ 11672 | SAV0758 | 54 | 90 | | |
| ΦNΞ 05522 | SAV0759 | 105 | | | |
| ΦNΞ 04223 | SAV0760 | 105 | | | |
| ΦNΞ 03114 | SAV0761 | 125 | | | |
| ΦNΞ 00106 | SAV0761 | 90 | | | |
| ΦNΞ 02399 | SAV0762 | 105 | | | |
| ΦNΞ 05151 | SAV0763 | 100 | | | |
| ΦNΞ 13116 | SAV0768 | 22 | | | |
| ΦNΞ 00102 | SAV0769 | 95 | | | |
| ΦNΞ 03043 | SAV0769 | 88 | | | |
| ΦNΞ 15277 | SAV0770 | 57 | 93 | | |
| ΦNΞ 03118 | SAV0775 | 125 | | | |
| ΦNΞ 14436 | SAV0779 | 97 | | | |
| ΦNΞ 04111 | SAV0800 | 79 | | | |
| ΦNΞ 03793 | SAV0803 | 112 | | | |
| ΦNΞ 02513 | SAV0804 | 100 | | | |
| ΦNΞ 02360 | SAV0805 | 100 | | | |
| ΦNΞ 12858 | SAV0808 | 43 | 82 | | 98 |
| ΦNΞ 09646 | SAV0809 | 105 | | | |
| ΦNΞ 06882 | SAV0810 | 100 | | | |
| ΦNΞ 05280 | SAV0811 | 100 | | | |
| ΦNΞ 06933 | SAV0812 | 87 | | | |
| ΦNΞ 09823 | SAV0815 | 95 | 65 | 56 | 93 |
| ΦNΞ 03877 | SAV0815 | 97 | | | |
| ΦNΞ 15315 | SAV0819 | 73 | 104 | | |
| ΦNΞ 07534 | SAV0822 | 63 | 82 | | 98 |
| ΦNΞ 11279 | SAV0825 | 95 | | | |
| ΦNΞ 06610 | SAV0826 | 73 | 107 | | |
| ΦNΞ 12871 | SAV0830 | 57 | 100 | | |
| ΦNΞ 06050 | SAV0830 | 138 | | | |
| ΦNΞ 02894 | SAV0835 | 100 | 80 | 85 | 98 |
| ΦNΞ 05142 | SAV0837 | 95 | | | |
| ΦNΞ 02404 | SAV0838 | 105 | | | |
| ΦNΞ 00555 | SAV0838 | 80 | | | |
| ΦNΞ 12909 | SAV0839 | 90 | | | |
| ΦNΞ 04279 | SAV0841 | 21 | 30 | 13 | 10 |
| ΦNΞ 03788 | SAV0845 | 118 | | | |
| ΦNΞ 09814 | SAV0848 | 105 | | | |
| ΦNΞ 00225 | SAV0849 | 100 | | | |
| ΦNΞ 07262 | SAV0855 | 27 | 36 | | 70 |
| ΦNΞ 03416 | SAV0855 | 84 | | | |
| ΦNΞ 12671 | SAV0858 | 57 | 29 | | |
| ΦNΞ 04000 | SAV0861* | 95 | | | |
| ΦNΞ 12478 | SAV0862 | 7 | 21 | | 96 |
| ΦNΞ 06544 | SAV0863 | 82 | 104 | | |
| ΦNΞ 08772 | SAV0866 | 28 | 22.7 | | |
| ΦNΞ 12985 | SAV0868* | 98 | | | |
| ΦNΞ 04743 | SAV0875 | 100 | | | |
| ΦNΞ 03004 | SAV0876 | 38 | 15 | 27 | 17 |
| ΦNΞ 01478 | SAV0877 | 85 | | | |
| ΦNΞ 04372 | SAV0881 | 118 | | | |
| ΦNΞ 03060 | SAV0882 | 75 | 60 | 65 | 100 |
| ΦNΞ 00024 | SAV0883 | 50 | 100 | | |
| ΦNΞ 05312 | SAV0885 | 95 | | | |
| ΦNΞ 00046 | SAV0886 | 81 | | | |
| ΦNΞ 08764 | SAV0886 | 79 | | | |
| ΦNΞ 10805 | SAV0887 | 90 | | | |
| ΦNΞ 03820 | SAV0887 | 90 | | | |
| ΦNΞ 00084 | SAV0888 | 85 | | | |
| ΦNΞ 03685 | SAV0889 | 76 | | | |
| ΦNΞ 00294 | SAV0890 | 95 | | | |
| ΦNΞ 09919 | SAV0891 | 105 | | | |
| ΦNΞ 00667 | SAV0892 | 80 | | | |
| ΦNΞ 11287 | SAV0893 | 55 | 35.1 | | |
| ΦNΞ 02185 | SAV0894 | 100 | | | |
| ΦNΞ 03655 | SAV0895 | 100 | | | |
| ΦNΞ 02232 | SAV0896 | 89 | | | |
| ΦNΞ 03687 | SAV0897 | 47 | 100 | | |
| ΦNΞ 10971 | SAV0898 | 82 | | | |
| ΦNΞ 04734 | SAV0898 | 100 | | | |
| ΦNΞ 06680 | SAV0898 | 163 | | | |
| ΦNΞ 09552 | SAV0900 | 55 | 100 | | |
| ΦNΞ 03708 | SAV0901 | 82 | | | |
| ΦNΞ 00064 | SAV0901 | 81 | | | |
| ΦNΞ 00226 | SAV0902 | 100 | | | |
| ΦNΞ 02017 | SAV0903 | 53 | 95 | | |
| ΦNΞ 01552 | SAV0904 | 104 | | | |
| ΦNΞ 03093 | SAV0904 | 94 | | | |
| ΦNΞ 00637 | SAV0905 | 90 | | | |
| ΦNΞ 07080 | SAV0906 | 67 | 89 | | |
| ΦNΞ 06695 | SAV0908 | 96 | | | |
| ΦNΞ 03088 | SAV0909 | 113 | | | |
| ΦNΞ 01519 | SAV0909 | 104 | | | |
| ΦNΞ 03217 | SAV0910 | 100 | | | |
| ΦNΞ 00672 | SAV0910 | 70 | | | |
| ΦNΞ 01580 | SAV0911 | 85 | | | |
| ΦNΞ 06605 | SAV0912 | 166 | | | |
| ΦNΞ 00479 | SAV0913 | 75 | | | |
| ΦNΞ 12527 | SAV0920 | 47 | 93 | | |
| ΦNΞ 04333 | SAV0921 | 100 | | | |
| ΦNΞ 06690 | SAV0922 | 175 | | | |
| ΦNΞ 03068 | SAV0922 | 106 | | | |
| ΦNΞ 01629 | SAV0922 | 105 | | | |
| ΦNΞ 01811 | SAV0923 | 89 | | | |
| ΦNΞ 00703 | SAV0924 | 15 | 100 | | |
| ΦNΞ 00769 | SAV0927 | 35 | 100 | | |
| ΦNΞ 05344 | SAV0928 | 100 | | | |
| ΦNΞ 01968 | SAV0929 | 95 | | | |
| ΦNΞ 00653 | SAV0930 | 85 | | | |
| ΦNΞ 12076 | SAV0933 | 54 | 100 | | |
| ΦNΞ 09346 | SAV0935 | 100 | | | |
| ΦNΞ 04728 | SAV0937 | 100 | | | |
| ΦNΞ 00231 | SAV0938 | 100 | | | |
| ΦNΞ 13891 | SAV0939 | 100 | | | |
| ΦNΞ 10876 | SAV0940 | 72 | 45.5 | 29.8 | |
| ΦNΞ 01739 | SAV0941 | 105 | | | |
| ΦNΞ 05012 | SAV0942 | 85 | | | |
| ΦNΞ 11584 | SAV0943 | 63 | 93 | | |
| ΦNΞ 05437 | SAV0944 | 95 | 65 | 47 | 25 |
| ΦNΞ 04008 | SAV0945 | 75 | | | |
| ΦNΞ 09242 | SAV0956 | 93 | | | |
| ΦNΞ 03954 | SAV0957 | 100 | | | |
| ΦNΞ 02830 | SAV0958 | 106 | | | |
| ΦNΞ 00806 | SAV0959 | 31 | 100 | | |
| ΦNΞ 04599 | SAV0960 | 76 | | | |
| ΦNΞ 08885 | SAV0962 | 98 | | | |
| ΦNΞ 00327 | SAV0963 | 80 | | | |
| ΦNΞ 02078 | SAV0968 | 63 | 100 | | |
| ΦNΞ 02923 | SAV0969 | 94 | | | |
| ΦNΞ 11697 | SAV0970 | 33 | 103 | | |
| ΦNΞ 05620 | SAV0974 | 105 | | | |
| ΦNΞ 03545 | SAV0976 | 100 | | | |
| ΦNΞ 00694 | SAV0977 | 35 | 100 | | |
| ΦNΞ 05299 | SAV0978 | 100 | | | |
| ΦNΞ 05409 | SAV0979 | 100 | | | |
| ΦNΞ 01532 | SAV0981 | 110 | | | |
| ΦNΞ 04047 | SAV0985 | 100 | | | |
| ΦNΞ 02880 | SAV0986 | 113 | | | |
| ΦNΞ 02280 | SAV0987 | 100 | | | |
| ΦNΞ 14304 | SAV0988 | 100 | | | |
| ΦNΞ 04084 | SAV0989 | 94 | | | |
| ΦNΞ 02040 | SAV0990 | 105 | | | |
| ΦNΞ 00303 | SAV0991 | 90 | | | |
| ΦNΞ 01951 | SAV0992 | 100 | | | |
| ΦNΞ 05621 | SAV0993 | 105 | | | |
| ΦNΞ 03876 | SAV0994 | 100 | | | |
| ΦNΞ 10629 | SAV0995 | 90 | | | |
| ΦNΞ 05436 | SAV0998 | 105 | | | |
| ΦNΞ 04804 | SAV1000 | 100 | | | |
| ΦNΞ 00315 | SAV1000 | 100 | | | |
| ΦNΞ 05368 | SAV1000 | 100 | | | |
| ΦNΞ 06682 | SAV1002 | 188 | | | |
| ΦNΞ 02696 | SAV1004 | 125 | | | |
| ΦNΞ 04841 | SAV1008 | 100 | | | |
| ΦNΞ 04961 | SAV1009 | 100 | | | |
| ΦNΞ 00142 | SAV1010 | 70 | 95 | | |
| ΦNΞ 00126 | SAV1012 | 65 | 90 | 37 | 25 |
| ΦNΞ 10663 | SAV1013 | 97 | | | |
| ΦNΞ 08774 | SAV1014 | 94 | | | |
| ΦNΞ 06998 | SAV1019 | 70 | 96 | | |
| ΦNΞ 04400 | SAV1023 | 71 | | | 93 |
| ΦNΞ 02935 | SAV1024 | 125 | | | |
| ΦNΞ 05509 | SAV1025 | 105 | | | |
| ΦNΞ 03801 | SAV1028 | 100 | | | |
| ΦNΞ 00658 | SAV1028* | 90 | | | |
| ΦNΞ 04348 | SAV1030 | 112 | | | |
| ΦNΞ 05080 | SAV1031 | 95 | | | |
| ΦNΞ 02673 | SAV1032 | 95 | | | |
| ΦNΞ 02898 | SAV1033 | 119 | | | |
| ΦNΞ 03633 | SAV1035 | 88 | | | |
| ΦNΞ 13696 | SAV1037 | 57 | 100 | | |
| ΦNΞ 00261 | SAV1039 | 60 | | | |
| ΦNΞ 03175 | SAV1039 | 106 | | | |
| ΦNΞ 05328 | SAV1040 | 100 | | | |
| ΦNΞ 09687 | SAV1044 | 105 | | | |
| ΦNΞ 04732 | SAV1046 | 100 | | | |
| ΦNΞ 01572 | SAV1047 | 91 | | | |
| ΦNΞ 13070 | SAV1048 | 98 | | | |
| ΦNΞ 09873 | SAV1049 | 105 | | | |
| ΦNΞ 02744 | SAV1050 | 125 | | | |
| ΦNΞ 06238 | SAV1052 | 22 | 89.7 | | |
| ΦNΞ 04970 | SAV1053 | 100 | | | |
| ΦNΞ 06233 | SAV1054 | 144 | | | |
| ΦNΞ 10201 | SAV1054 | 105 | 59 | 75 | 100 |
| ΦNΞ 05261 | SAV1057 | 90 | | | |
| ΦNΞ 02302 | SAV1060 | 105 | | | |
| ΦNΞ 05543 | SAV1062 | 105 | | | |
| ΦNΞ 02380 | SAV1064 | 100 | | | |
| ΦNΞ 05091 | SAV1065 | 100 | | | |
| ΦNΞ 04806 | SAV1065 | 100 | | | |
| ΦNΞ 02924 | SAV1066 | 125 | | | |
| ΦNΞ 01626 | SAV1066 | 105 | | | |
| ΦNΞ 12894 | SAV1067 | 100 | | | |
| ΦNΞ 05399 | SAV1068 | 105 | | | |
| ΦNΞ 03038 | SAV1070 | 125 | | | |
| ΦNΞ 03863 | SAV1071 | 100 | | | |
| ΦNΞ 04103 | SAV1073 | 118 | | | |
| ΦNΞ 03896 | SAV1074 | 100 | | | |
| ΦNΞ 03789 | SAV1075 | 118 | | | |
| ΦNΞ 05649 | SAV1076 | 105 | | | |
| ΦNΞ 13746 | SAV1077 | 102 | | | |
| ΦNΞ 00570 | SAV1081 | 85 | | | |
| ΦNΞ 00870 | SAV1084 | 56 | 100 | | |
| ΦNΞ 06439 | SAV1086 | 153 | | | |
| ΦNΞ 09917 | SAV1088 | 105 | | | |
| ΦNΞ 01837 | SAV1089 | 89 | | | |
| ΦNΞ 04761 | SAV1092 | 95 | | | |
| ΦNΞ 10868 | SAV1094 | 0 | 26 | | 22 |
| ΦNΞ 04312 | SAV1098 | 68 | | | |
| ΦNΞ 11872 | SAV1100 | 75 | 97 | | |
| ΦNΞ 12133 | SAV1101 | 46 | 100 | | |
| ΦNΞ 02669 | SAV1102 | 100 | | | |
| ΦNΞ 06164 | SAV1104 | 134 | | | |
| ΦNΞ 03662 | SAV1105 | 76 | | | |
| ΦNΞ 12645 | SAV1108 | 100 | | | |
| ΦNΞ 02729 | SAV1109 | 125 | | | |
| ΦNΞ 04029 | SAV1109 | 95 | | | |
| ΦNΞ 06499 | SAV1110 | 141 | | | |
| ΦNΞ 04166 | SAV1111 | 105 | | | |
| ΦNΞ 01941 | SAV1111 | 105 | | | |
| ΦNΞ 09768 | SAV1112 | 100 | 100 | | |
| ΦNΞ 04475 | SAV1114 | 47 | 95 | | |
| ΦNΞ 03778 | SAV1114 | 59 | 100 | | |
| ΦNΞ 14423 | SAV1115 | 100 | | | |
| ΦNΞ 02477 | SAV1117 | 79 | 100 | | |
| ΦNΞ 00441 | SAV1118 | 25 | 95 | | |
| ΦNΞ 04298 | SAV1121 | 105 | | | |
| ΦNΞ 03807 | SAV1121 | 90 | | | |
| ΦNΞ 04820 | SAV1122 | 75 | 90 | | |
| ΦNΞ 12232 | SAV1123 | 96 | | | |
| ΦNΞ 11795 | SAV1126 | 118 | | | |
| ΦNΞ 06230 | SAV1127 | 166 | | | |
| ΦNΞ 04990 | SAV1129 | 95 | | | |
| ΦNΞ 01954 | SAV1130 | 105 | | | |
| ΦNΞ 09284 | SAV1131 | 103 | | | |
| ΦNΞ 03054 | SAV1132 | 69 | | | |
| ΦNΞ 04382 | SAV1132 | 112 | | | |
| ΦNΞ 13865 | SAV1133 | 100 | | | |
| ΦNΞ 12849 | SAV1134 | 93 | | | |
| ΦNΞ 03811 | SAV1135 | 75 | | | |
| ΦNΞ 01454 | SAV1135 | 104 | | | |
| ΦNΞ 06585 | SAV1136 | 90 | 100 | | |
| ΦNΞ 05067 | SAV1136 | 85 | | | |
| ΦNΞ 04124 | SAV1137 | 100 | | | |
| ΦNΞ 04007 | SAV1142 | 70 | | | |
| ΦNΞ 00668 | SAV1143 | 75 | | | |
| ΦNΞ 05614 | SAV1144 | 105 | | | |
| ΦNΞ 05356 | SAV1146 | 100 | | | |
| ΦNΞ 04650 | SAV1146 | 112 | | | |
| ΦNΞ 01442 | SAV1148 | 104 | | | |
| ΦNΞ 04085 | SAV1150 | 118 | | | |
| ΦNΞ 02844 | SAV1152 | 100 | | | |
| ΦNΞ 11877 | SAV1153 | 13 | 97 | | |
| ΦNΞ 13803 | SAV1157 | 100 | | | |
| ΦNΞ 03597 | SAV1158 | 100 | | | |
| ΦNΞ 11961 | SAV1159 | 92 | | | |
| ΦNΞ 02140 | SAV1163 | 89 | | | |
| ΦNΞ 01440 | SAV1166 | 122 | | | 101 |
| ΦNΞ 10229 | SAV1168 | 105 | | | |
| ΦNΞ 05014 | SAV1169 | 100 | | | |
| ΦNΞ 12598 | SAV1170 | 97 | | | |
| ΦNΞ 04829 | SAV1171 | 95 | | | |
| ΦNΞ 04507 | SAV1172 | 118 | | | |
| ΦNΞ 00372 | SAV1173 | 75 | | | |
| ΦNΞ 11641 | SAV1174 | 121 | | | |
| ΦNΞ 09093 | SAV1175 | 97 | | | |
| ΦNΞ 04044 | SAV1177 | 76 | 30 | 50 | 100 |
| ΦNΞ 10949 | SAV1190 | 86 | 64.9 | | |
| ΦNΞ 12315 | SAV1191 | 97 | | | |
| ΦNΞ 00715 | SAV1194 | 65 | | | |
| ΦNΞ 00656 | SAV1201 | 30 | 5 | | |
| ΦNΞ 00678 | SAV1202 | 35 | 20 | | |
| ΦNΞ 02887 | SAV1203 | 88 | 5 | | 88 |
| ΦNΞ 04659 | SAV1203 | 12 | 80 | | |
| ΦNΞ 12249 | SAV1204* | 42 | 103 | | |
| ΦNΞ 02904 | SAV1205 | 56 | 5 | | 94 |
| ΦNΞ 03984 | SAV1206 | 90 | | | |
| ΦNΞ 04888 | SAV1207 | 100 | | | |
| ΦNΞ 09798 | SAV1208 | 105 | | | |
| ΦNΞ 09735 | SAV1213 | 105 | | | |
| ΦNΞ 00197 | SAV1217 | 100 | | | |
| ΦNΞ 00032 | SAV1218 | 81 | | | |
| ΦNΞ 14673 | SAV1219 | 43 | 100 | | |
| ΦNΞ 03940 | SAV1220 | 95 | | | |
| ΦNΞ 12600 | SAV1221 | 30 | 14 | | |
| ΦNΞ 11428 | SAV1224 | 117 | | | |
| ΦNΞ 00020 | SAV1226 | 25 | 100 | | |
| ΦNΞ 11173 | SAV1227 | 27 | 24 | | 84 |
| ΦNΞ 10529 | SAV1234 | 64 | 79 | | 96 |
| ΦNΞ 11999 | SAV1240 | 104 | | | |
| ΦNΞ 09294 | SAV1241 | 103 | | | |
| ΦNΞ 05179 | SAV1242 | 90 | | | |
| ΦNΞ 01541 | SAV1244 | 91 | | | |
| ΦNΞ 00594 | SAV1247 | 60 | 100 | | |
| ΦNΞ 02920 | SAV1248 | 88 | 80 | 78 | 98 |
| ΦNΞ 06190 | SAV1249 | 159 | | | |
| ΦNΞ 03407 | SAV1251 | 89 | | | |
| ΦNΞ 09425 | SAV1270 | 132 | | | |
| ΦNΞ 02315 | SAV1271 | 100 | | | |
| ΦNΞ 05451 | SAV1274 | 105 | | | |
| ΦNΞ 10926 | SAV1278 | 27 | 97 | | |
| ΦNΞ 14030 | SAV1279 | 100 | | | |
| ΦNΞ 04737 | SAV1280 | 100 | | | |
| ΦNΞ 01812 | SAV1281 | 100 | | | |
| ΦNΞ 09773 | SAV1284 | 100 | 100 | | |
| ΦNΞ 00206 | SAV1292 | 20 | 100 | | |
| ΦNΞ 09545 | SAV1294 | 32 | 107 | | |
| ΦNΞ 04775 | SAV1296 | 100 | | | |
| ΦNΞ 00866 | SAV1296 | 72 | | | |
| ΦNΞ 01512 | SAV1297 | 79 | | | |
| ΦNΞ 00722 | SAV1298 | 70 | | | |
| ΦNΞ 11481 | SAV1300 | 42 | 48 | | 98 |
| ΦNΞ 05496 | SAV1301 | 105 | | | |
| ΦNΞ 02025 | SAV1302 | 100 | | | |
| ΦNΞ 09695 | SAV1303 | 105 | | | |
| ΦNΞ 06616 | SAV1305 | 178 | | | |
| ΦNΞ 04164 | SAV1306 | 100 | | | |
| ΦNΞ 00270 | SAV1307 | 90 | | | |
| ΦNΞ 02357 | SAV1308 | 105 | | | |
| ΦNΞ 01862 | SAV1314 | 100 | | | |
| ΦNΞ 03808 | SAV1315 | 85 | | | |
| ΦNΞ 00201 | SAV1317 | 95 | | | |
| ΦNΞ 04854 | SAV1318 | 100 | | | |
| ΦNΞ 09053 | SAV1319 | 90 | 100 | | |
| ΦNΞ 12683 | SAV1320 | 63 | 104 | | |
| ΦNΞ 01824 | SAV1321 | 105 | | | |
| ΦNΞ 11466 | SAV1322 | 46 | 103 | | |
| ΦNΞ 00021 | SAV1323 | 94 | | | |
| ΦNΞ 03258 | SAV1324 | 95 | | | |
| ΦNΞ 04891 | SAV1326 | 100 | | | |
| ΦNΞ 03196 | SAV1327 | 106 | | | |
| ΦNΞ 11942 | SAV1328 | 104 | | | |
| ΦNΞ 12625 | SAV1329 | 53 | 71 | | 100 |
| ΦNΞ 09336 | SAV1330 | 82 | | | |
| ΦNΞ 10079 | SAV1331 | 105 | | | |
| ΦNΞ 00210 | SAV1332 | 45 | 100 | | |
| ΦNΞ 03920 | SAV1333 | 100 | | | |
| ΦNΞ 12739 | SAV1335 | 80 | 75 | | 100 |
| ΦNΞ 06121 | SAV1336 | 169 | | | |
| ΦNΞ 03569 | SAV1337 | 89 | | | |
| ΦNΞ 05032 | SAV1338 | 100 | | | |
| ΦNΞ 04545 | SAV1340 | 106 | | | |
| ΦNΞ 10517 | SAV1341 | 50 | 86 | | 100 |
| ΦNΞ 09392 | SAV1344 | 105 | | | |
| ΦNΞ 05227 | SAV1345 | 100 | | | |
| ΦNΞ 00220 | SAV1346 | 30 | 85 | 98 | |
| ΦNΞ 06464 | SAV1349 | 90 | 104 | | |
| ΦNΞ 00263 | SAV1350 | 25 | 50 | | 58 |
| ΦNΞ 06317 | SAV1351 | 147 | | | |
| ΦNΞ 11868 | SAV1355 | 21 | 34 | | 100 |
| ΦNΞ 09110 | SAV1356 | 72 | 86.4 | 63.2 | |
| ΦNΞ 11405 | SAV1357 | 132 | | | |
| ΦNΞ 08767 | SAV1361 | 103 | | | |
| ΦNΞ 11797 | SAV1362* | 50 | 21.1 | | |
| ΦNΞ 04469 | SAV1364 | 53 | 95 | | |
| ΦNΞ 09750 | SAV1365 | 100 | 100 | | |
| ΦNΞ 03998 | SAV1366 | 90 | | | |
| ΦNΞ 00553 | SAV1367 | 100 | | | |
| ΦNΞ 06482 | SAV1368 | 134 | | | |
| ΦNΞ 03894 | SAV1369 | 100 | | | |
| ΦNΞ 10240 | SAV1370 | 84 | 65 | | 72 |
| ΦNΞ 01453 | SAV1371 | 104 | | | |
| ΦNΞ 11372 | SAV1372 | 82 | 71.9 | | |
| ΦNΞ 03755 | SAV1373 | 118 | | | |
| ΦNΞ 00727 | SAV1373 | 85 | | | |
| ΦNΞ 09532 | SAV1377 | 27 | 100 | | |
| ΦNΞ 00134 | SAV1379 | 85 | | | |
| ΦNΞ 04936 | SAV1379 | 95 | | | |
| ΦNΞ 12518 | SAV1380 | 90 | | | |
| ΦNΞ 05051 | SAV1381 | 100 | | | |
| ΦNΞ 04189 | SAV1382 | 58 | 45 | 75 | 100 |
| ΦNΞ 00562 | SAV1383 | 80 | | | |
| ΦNΞ 03893 | SAV1384 | 85 | | | |
| ΦNΞ 00589 | SAV1385 | 80 | | | |
| ΦNΞ 05321 | SAV1386 | 100 | | | |
| ΦNΞ 05589 | SAV1387 | 105 | | | |
| ΦNΞ 06387 | SAV1388 | 153 | | | |
| ΦNΞ 01667 | SAV1389 | 84 | | | |
| ΦNΞ 02833 | SAV1389 | 125 | | | |
| ΦNΞ 04987 | SAV1391 | 100 | | | |
| ΦNΞ 04444 | SAV1392 | 100 | | | |
| ΦNΞ 09077 | SAV1393 | 90 | | | |
| ΦNΞ 04943 | SAV1394 | 95 | | | |
| ΦNΞ 02057 | SAV1395 | 100 | | | |
| ΦNΞ 00844 | SAV1396 | 41 | 90 | 97 | |
| ΦNΞ 12571 | SAV1397 | 97 | | | |
| ΦNΞ 11121 | SAV1399 | 132 | | | |
| ΦNΞ 10233 | SAV1400 | 105 | | | |
| ΦNΞ 05537 | SAV1401 | 105 | | | |
| ΦNΞ 08971 | SAV1404 | 80 | 100 | | |
| ΦNΞ 09592 | SAV1405 | 86 | | | |
| ΦNΞ 00420 | SAV1407 | 80 | | | |
| ΦNΞ 03005 | SAV1408 | 100 | | | |
| ΦNΞ 03580 | SAV1408 | 32 | 15 | 27 | 98 |
| ΦNΞ 03792 | SAV1411 | 112 | | | |
| ΦNΞ 04792 | SAV1412 | 90 | 100 | | |
| ΦNΞ 04120 | SAV1416 | 53 | 95 | | |
| ΦNΞ 09941 | SAV1420 | 105 | | | |
| ΦNΞ 02406 | SAV1423 | 105 | | | |
| ΦNΞ 05309 | SAV1425 | 95 | | | |
| ΦNΞ 05515 | SAV1429 | 95 | 100 | | |
| ΦNΞ 02155 | SAV1432 | 68 | 100 | | |
| ΦNΞ 11448 | SAV1433 | 58 | 66 | | 100 |
| ΦNΞ 04384 | SAV1434 | 118 | | | |
| ΦNΞ 00288 | SAV1435 | 85 | | | |
| ΦNΞ 02417 | SAV1435 | 105 | | | |
| ΦNΞ 03648 | SAV1435 | 94 | | | |
| ΦNΞ 13073 | SAV1436 | 92 | | | |
| ΦNΞ 03505 | SAV1436 | 89 | | | |
| ΦNΞ 02311 | SAV1437 | 105 | | | |
| ΦNΞ 09822 | SAV1437 | 100 | 95 | | |
| ΦNΞ 03573 | SAV1438 | 74 | | | |
| ΦNΞ 01628 | SAV1439 | 105 | | | |
| ΦNΞ 10883 | SAV1440 | 97 | | | |
| ΦNΞ 04669 | SAV1441 | 100 | | | |
| ΦNΞ 10919 | SAV1442 | 62 | 50 | 49.1 | |
| ΦNΞ 05628 | SAV1443 | 105 | | | |
| ΦNΞ 10463 | SAV1444 | 100 | | | |
| ΦNΞ 02660 | SAV1448 | 100 | | | |
| ΦNΞ 00061 | SAV1451 | 63 | 100 | | |
| ΦNΞ 11116 | SAV1454 | 123 | | | |
| ΦNΞ 01561 | SAV1455 | 67 | 100 | 97 | |
| ΦNΞ 09834 | SAV1455 | 100 | 85 | | 97 |
| ΦNΞ 10521 | SAV1458 | 41 | 93 | | |
| ΦNΞ 14032 | SAV1459 | 100 | | | |
| ΦNΞ 04132 | SAV1461 | 105 | | | |
| ΦNΞ 01471 | SAV1463 | 98 | | | |
| ΦNΞ 00200 | SAV1469 | 60 | 95 | | |
| ΦNΞ 09236 | SAV1476 | 96 | | | |
| ΦNΞ 00664 | SAV1480 | 75 | | | |
| ΦNΞ 02088 | SAV1480 | 105 | | | |
| ΦNΞ 01733 | SAV1482 | 95 | | | |
| ΦNΞ 04755 | SAV1483 | 100 | | | |
| ΦNΞ 09828 | SAV1484 | 105 | | | |
| ΦNΞ 00334 | SAV1485 | 100 | | | |
| ΦNΞ 09669 | SAV1488 | 105 | | | |
| ΦNΞ 06275 | SAV1489 | 134 | 100 | | |
| ΦNΞ 08794 | SAV1489* | 86 | | | |
| ΦNΞ 12844 | SAV1491 | 97 | | | |
| ΦNΞ 00634 | SAV1493 | 85 | | | |
| ΦNΞ 08902 | SAV1494 | 92 | | | |
| ΦNΞ 02742 | SAV1495 | 119 | | | |
| ΦNΞ 10457 | SAV1496 | 93 | | | |
| ΦNΞ 03765 | SAV1499 | 71 | | | |
| ΦNΞ 08669 | SAV1500 | 86 | | | |
| ΦNΞ 04692 | SAV1501 | 100 | | | |
| ΦNΞ 03207 | SAV1502 | 105 | | | |
| ΦNΞ 09842 | SAV1503 | 105 | | | |
| ΦNΞ 03915 | SAV1506 | 95 | | | |
| ΦNΞ 10195 | SAV1507 | 105 | | | |
| ΦNΞ 04920 | SAV1508 | 100 | | | |
| ΦNΞ 01616 | SAV1509 | 53 | 100 | | |
| ΦNΞ 00153 | SAV1512 | 90 | | | |
| ΦNΞ 05030 | SAV1513 | 95 | | | |
| ΦNΞ 09950 | SAV1514 | 105 | | | |
| ΦNΞ 10858 | SAV1515 | 64 | 10 | | |
| ΦNΞ 13468 | SAV1517 | 100 | | | |
| ΦNΞ 10390 | SAV1518 | 9 | | | |
| ΦNΞ 09439 | SAV1519 | 45 | 64 | | |
| ΦNΞ 00598 | SAV1520 | 50 | 95 | | |
| ΦNΞ 00218 | SAV1521 | 65 | 95 | | |
| ΦNΞ 01978 | SAV1523 | 26 | 95 | | |
| ΦNΞ 13764 | SAV1524 | 98 | | | |
| ΦNΞ 09368 | SAV1529 | 105 | | | |
| ΦNΞ 02082 | SAV1535 | 95 | | | |
| ΦNΞ 01569 | SAV1536 | 104 | | | |
| ΦNΞ 04366 | SAV1539 | 112 | | | |
| ΦNΞ 09937 | SAV1540 | 105 | | | |
| ΦNΞ 09268 | SAV1541 | 100 | | | |
| ΦNΞ 03679 | SAV1543 | 106 | | | |
| ΦNΞ 04447 | SAV1544 | 82 | | | |
| ΦNΞ 05124 | SAV1545 | 95 | | | |
| ΦNΞ 10927 | SAV1546 | 68 | | | |
| ΦNΞ 01495 | SAV1547 | 49 | 100 | | |
| ΦNΞ 09080 | SAV1549 | 55 | 96 | | |
| ΦNΞ 00130 | SAV1550 | 85 | | | |
| ΦNΞ 11029 | SAV1555 | 127 | | | |
| ΦNΞ 00845 | SAV1556 | 82 | | | |
| ΦNΞ 05095 | SAV1557 | 100 | | | |
| ΦNΞ 10627 | SAV1558 | 79 | | | |
| ΦNΞ 03909 | SAV1563 | 100 | | | |
| ΦNΞ 10839 | SAV1571 | 86 | | | |
| ΦNΞ 06393 | SAV1572 | 153 | | | |
| ΦNΞ 10437 | SAV1574 | 103 | | | |
| ΦNΞ 09721 | SAV1574 | 105 | | | |
| ΦNΞ 05130 | SAV1576 | 100 | | | |
| ΦNΞ 00034 | SAV1577 | 69 | | | |
| ΦNΞ 00481 | SAV1583 | 85 | | | |
| ΦNΞ 15237 | SAV1585 | 70 | 100 | | |
| ΦNΞ 03153 | SAV1588 | 19 | 35 | 43 | 98 |
| ΦNΞ 15341 | SAV1589 | 90 | | | |
| ΦNΞ 04436 | SAV1590 | 76 | | | |
| ΦNΞ 03417 | SAV1591 | 79 | | | |
| ΦNΞ 13165 | SAV1593 | 67 | 104 | | |
| ΦNΞ 01788 | SAV1596 | 74 | 100 | | |
| ΦNΞ 09942 | SAV1596 | 105 | | | |
| ΦNΞ 14659 | SAV1600 | 87 | | | |
| ΦNΞ 10658 | SAV1601 | 90 | | | |
| ΦNΞ 05136 | SAV1603 | 100 | | | |
| ΦNΞ 03405 | SAV1604 | 105 | | | |
| ΦNΞ 00048 | SAV1606 | 81 | | | |
| ΦNΞ 02005 | SAV1606 | 84 | | | |
| ΦNΞ 00810 | SAV1607 | 87 | | | |
| ΦNΞ 05512 | SAV1609 | 105 | | | |
| ΦNΞ 13426 | SAV1610 | 96 | | | |
| ΦNΞ 02786 | SAV1611 | 125 | | | |
| ΦNΞ 10251 | SAV1612 | 105 | | | |
| ΦNΞ 05586 | SAV1613 | 105 | | | |
| ΦNΞ 02797 | SAV1614 | 125 | | | |
| ΦNΞ 09933 | SAV1614 | 105 | | | |
| ΦNΞ 13374 | SAV1616 | 92 | | | |
| ΦNΞ 13118 | SAV1617 | 51 | | | |
| ΦNΞ 01851 | SAV1619 | 16 | 70 | 22 | 63 |
| ΦNΞ 03924 | SAV1619 | 75 | 30 | 53 | 78 |
| ΦNΞ 12141 | SAV1620 | 38 | 76 | | 84 |
| ΦNΞ 00281 | SAV1623 | 95 | | | |
| ΦNΞ 10418 | SAV1624 | 103 | | | |
| ΦNΞ 09949 | SAV1625 | 95 | 90 | | |
| ΦNΞ 03264 | SAV1627 | 37 | 70 | 88 | 98 |
| ΦNΞ 11865 | SAV1628 | 88 | | | |
| ΦNΞ 02307 | SAV1632 | 105 | | | |
| ΦNΞ 12680 | SAV1635 | 97 | | | |
| ΦNΞ 00874 | SAV1636 | 67 | | | |
| ΦNΞ 02425 | SAV1638 | 95 | | | |
| ΦNΞ 03502 | SAV1638 | 105 | | | |
| ΦNΞ 03576 | SAV1639 | 89 | | | |
| ΦNΞ 02724 | SAV1640 | 119 | | | |
| ΦNΞ 08942 | SAV1645 | 51 | 79 | | 98 |
| ΦNΞ 04763 | SAV1651 | 100 | | | |
| ΦNΞ 13114 | SAV1660 | 102 | | | |
| ΦNΞ 04873 | SAV1661 | 100 | | | |
| ΦNΞ 09611 | SAV1664 | 105 | | | |
| ΦNΞ 00482 | SAV1665 | 85 | | | |
| ΦNΞ 00691 | SAV1667 | 40 | 100 | | |
| ΦNΞ 09787 | SAV1674 | 105 | | | |
| ΦNΞ 11361 | SAV1675 | 123 | | | |
| ΦNΞ 00815 | SAV1676 | 26 | 95 | | |
| ΦNΞ 01639 | SAV1677 | 21 | 35 | 18 | 48 |
| ΦNΞ 01982 | SAV1687 | 16 | 50 | 22 | |
| ΦNΞ 11111 | SAV1689 | 123 | | | |
| ΦNΞ 05527 | SAV1690 | 100 | | | |
| ΦNΞ 06260 | SAV1691 | 169 | | | |
| ΦNΞ 03695 | SAV1692 | 47 | 90 | | |
| ΦNΞ 14551 | SAV1693 | 100 | | | |
| ΦNΞ 05271 | SAV1694 | 70 | 90 | | |
| ΦNΞ 06106 | SAV1695 | 188 | | | |
| ΦNΞ 13092 | SAV1697 | 100 | | | |
| ΦNΞ 01819 | SAV1706 | 16 | 85 | 73 | 100 |
| ΦNΞ 14013 | SAV1707 | 80 | 96 | | |
| ΦNΞ 10478 | SAV1708 | 91 | | | |
| ΦNΞ 02179 | SAV1709 | 105 | | | |
| ΦNΞ 10024 | SAV1710 | 105 | | | |
| ΦNΞ 09686 | SAV1712 | 105 | | | |
| ΦNΞ 09756 | SAV1713 | 105 | | | |
| ΦNΞ 12153 | SAV1714 | 54 | 97 | | |
| ΦNΞ 00831 | SAV1716 | 46 | 100 | | |
| ΦNΞ 13546 | SAV1718 | 102 | | | |
| ΦNΞ 09295 | SAV1721 | 103 | | | |
| ΦNΞ 06110 | SAV1722 | 144 | | | |
| ΦNΞ 02692 | SAV1723 | 119 | | | |
| ΦNΞ 05575 | SAV1724 | 105 | | | |
| ΦNΞ 09771 | SAV1725 | 100 | 70 | 84 | 75 |
| ΦNΞ 01769 | SAV1726 | 100 | | | |
| ΦNΞ 05535 | SAV1728 | 100 | | | |
| ΦNΞ 05285 | SAV1730 | 75 | 80 | 109 | |
| ΦNΞ 06315 | SAV1731 | 163 | | | |
| ΦNΞ 00742 | SAV1731 | 100 | | | 94 |
| ΦNΞ 04341 | SAV1732 | 32 | 90 | | |
| ΦNΞ 00456 | SAV1733 | 100 | 100 | | |
| ΦNΞ 09642 | SAV1734 | 105 | | | |
| ΦNΞ 03717 | SAV1735 | 65 | | | |
| ΦNΞ 11822 | SAV1736 | 88 | | | |
| ΦNΞ 12369 | SAV1744 | 57 | 93 | | |
| ΦNΞ 09978 | SAV1745 | 105 | | | |
| ΦNΞ 13099 | SAV1746 | 100 | | | |
| ΦNΞ 05495 | SAV1747 | 100 | | | |
| ΦNΞ 00370 | SAV1750 | 70 | | | |
| ΦNΞ 06678 | SAV1751 | 92 | | | |
| ΦNΞ 06780 | SAV1752 | 60 | 96 | | |
| ΦNΞ 03027 | SAV1753 | 50 | 80 | 78 | 100 |
| ΦNΞ 01581 | SAV1755 | 91 | | | |
| ΦNΞ 01809 | SAV1756 | 105 | | | |
| ΦNΞ 04980 | SAV1758 | 100 | | | |
| ΦNΞ 00208 | SAV1758 | 92 | | | 104 |
| ΦNΞ 05106 | SAV1758 | 95 | | | |
| ΦNΞ 04981 | SAV1761 | 100 | | | |
| ΦNΞ 00074 | SAV1762 | 90 | | | |
| ΦNΞ 05156 | SAV1763 | 95 | | | |
| ΦNΞ 11740 | SAV1765 | 77 | | | |
| ΦNΞ 13818 | SAV1766 | 96 | | | |
| ΦNΞ 00862 | SAV1768 | 103 | | | |
| ΦNΞ 14759 | SAV1769 | 100 | | | |
| ΦNΞ 04855 | SAV1770 | 100 | | | |
| ΦNΞ 03534 | SAV1771 | 100 | | | |
| ΦNΞ 02541 | SAV1772 | 100 | | | |
| ΦNΞ 02899 | SAV1772 | 125 | | | |
| ΦNΞ 11851 | SAV1773 | 96 | | | |
| ΦNΞ 03743 | SAV1774 | 100 | | | |
| ΦNΞ 06358 | SAV1775 | 172 | | | |
| ΦNΞ 09741 | SAV1777 | 105 | | | |
| ΦNΞ 11323 | SAV1778 | 105 | | | |
| ΦNΞ 09124 | SAV1779 | 90 | 93 | | |
| ΦNΞ 00247 | SAV1780 | 85 | | | |
| ΦNΞ 04924 | SAV1784 | 100 | | | |
| ΦNΞ 02929 | SAV1785 | 63 | 95 | | |
| ΦNΞ 05157 | SAV1787 | 100 | | | |
| ΦNΞ 13264 | SAV1788 | 90 | 107 | | |
| ΦNΞ 01969 | SAV1791 | 16 | 5 | | 90 |
| ΦNΞ 05533 | SAV1793 | 105 | | | |
| ΦNΞ 13076 | SAV1794 | 100 | | | |
| ΦNΞ 13249 | SAV1796 | 100 | | | |
| ΦNΞ 11082 | SAV1798 | 64 | 86 | | |
| ΦNΞ 04503 | SAV1801 | 118 | | | |
| ΦNΞ 04108 | SAV1803 | 106 | | | |
| ΦNΞ 05478 | SAV1805 | 105 | | | |
| ΦNΞ 04948 | SAV1807 | 100 | | | |
| ΦNΞ 03260 | SAV1808 | 42 | 85 | 78 | 100 |
| ΦNΞ 05473 | SAV1808 | 100 | | | |
| ΦNΞ 06104 | SAV1809 | 169 | | | |
| ΦNΞ 02814 | SAV1810 | 125 | | | |
| ΦNΞ 11075 | SAV1811 | 100 | | | |
| ΦNΞ 12789 | SAV1813 | 97 | | | |
| ΦNΞ 01785 | SAV1814 | 105 | | | |
| ΦNΞ 03169 | SAV1815 | 69 | 90 | | |
| ΦNΞ 13601 | SAV1819 | 100 | | | |
| ΦNΞ 08984 | SAV1820 | 92 | | | |
| ΦNΞ 03521 | SAV1821 | 95 | | | |
| ΦNΞ 09153 | SAV1835 | 88 | 104 | | |
| ΦNΞ 01973 | SAV1838 | 105 | | | |
| ΦNΞ 11581 | SAV1839 | 54 | 76 | | 96 |
| ΦNΞ 09667 | SAV1840 | 105 | | | |
| ΦNΞ 09920 | SAV1842 | 105 | | | |
| ΦNΞ 00631 | SAV1843 | 70 | | | |
| ΦNΞ 03798 | SAV1844 | 118 | | | |
| ΦNΞ 04904 | SAV1846 | 95 | | | |
| ΦNΞ 06189 | SAV1848 | 84 | 100 | | |
| ΦNΞ 03790 | SAV1849 | 106 | | | |
| ΦNΞ 13245 | SAV1850 | 88 | 100 | | |
| ΦNΞ 01596 | SAV1851 | 12 | | | |
| ΦNΞ 03937 | SAV1851 | 25 | 30 | | 63 |
| ΦNΞ 00748 | SAV1852 | 85 | | | |
| ΦNΞ 11183 | SAV1854 | 82 | 64.9 | | |
| ΦNΞ 04023 | SAV1856 | 60 | 65 | 75 | 100 |
| ΦNΞ 09706 | SAV1857 | 105 | | | |
| ΦNΞ 09524 | SAV1858 | 32 | 96 | | |
| ΦNΞ 05062 | SAV1859 | 100 | | | |
| ΦNΞ 01520 | SAV1862 | 61 | 100 | | |
| ΦNΞ 05104 | SAV1863 | 90 | | | |
| ΦNΞ 15185 | SAV1864 | 97 | | | |
| ΦNΞ 01784 | SAV1865 | 105 | | | |
| ΦNΞ 04859 | SAV1866 | 95 | | | |
| ΦNΞ 00194 | SAV1868 | 95 | | | |
| ΦNΞ 03703 | SAV1870 | 35 | 70 | 97 | |
| ΦNΞ 12992 | SAV1871 | 100 | | | 98 |
| ΦNΞ 12899 | SAV1873 | 53 | 75 | | 96 |
| ΦNΞ 06284 | SAV1874 | 150 | | | |
| ΦNΞ 03774 | SAV1875 | 41 | 90 | | |
| ΦNΞ 09071 | SAV1877 | 76 | 114 | | |
| ΦNΞ 03400 | SAV1879 | 100 | | | |
| ΦNΞ 03575 | SAV1880 | 89 | | | |
| ΦNΞ 15208 | SAV1881 | 83 | 104 | | |
| ΦNΞ 06353 | SAV1883 | 91 | 72.4 | | |
| ΦNΞ 02993 | SAV1884 | 69 | 45 | 67 | 95 |
| ΦNΞ 03821 | SAV1885 | 70 | 70 | 68 | 98 |
| ΦNΞ 02960 | SAV1886 | 88 | 95 | | |
| ΦNΞ 08685 | SAV1887 | 100 | | | |
| ΦNΞ 11839 | SAV1889 | 113 | | | |
| ΦNΞ 13487 | SAV1890 | 102 | | | |
| ΦNΞ 06497 | SAV1893 | 144 | | | |
| ΦNΞ 03231 | SAV1894 | 105 | | | |
| ΦNΞ 04966 | SAV1896 | 95 | | | |
| ΦNΞ 00136 | SAV1897 | 100 | | | |
| ΦNΞ 00137 | SAV1897 | 100 | | | |
| ΦNΞ 10870 | SAV1897 | 93 | 55 | 97 | |
| ΦNΞ 11673 | SAV1902 | 79 | | | |
| ΦNΞ 11997 | SAV1903 | 38 | 72 | | 100 |
| ΦNΞ 01622 | SAV1906 | 95 | | | |
| ΦNΞ 01592 | SAV1907 | 55 | 100 | | |
| ΦNΞ 05144 | SAV1908 | 100 | | | |
| ΦNΞ 06249 | SAV1909 | 98 | | | |
| ΦNΞ 04478 | SAV1911 | 65 | 90 | | |
| ΦNΞ 04134 | SAV1914 | 95 | | | |
| ΦNΞ 09372 | SAV1915 | 68 | | | |
| ΦNΞ 00718 | SAV1916 | 65 | | | |
| ΦNΞ 02291 | SAV1917 | 89 | | | |
| ΦNΞ 04769 | SAV1918 | 35 | 40 | 73 | 82 |
| ΦNΞ 04846 | SAV1922 | 100 | | | |
| ΦNΞ 01723 | SAV1924 | 95 | | | |
| ΦNΞ 05468 | SAV1925 | 105 | | | |
| ΦNΞ 04883 | SAV1926 | 0 | 10 | 38 | 30 |
| ΦNΞ 11080 | SAV1927 | 41 | 45 | | 20 |
| ΦNΞ 01449 | SAV1928 | 67 | 35 | 23 | 15 |
| ΦNΞ 11264 | SAV1929 | 55 | 38.6 | | |
| ΦNΞ 01508 | SAV1932 | 49 | 90 | 55 | 97 |
| ΦNΞ 12158 | SAV1933 | 42 | 62 | | 98 |
| ΦNΞ 09620 | SAV1934 | 105 | | | |
| ΦNΞ 13110 | SAV1935 | 98 | | | |
| ΦNΞ 02967 | SAV1936 | 88 | | | |
| ΦNΞ 04149 | SAV1938 | 79 | | | |
| ΦNΞ 09659 | SAV1938 | 105 | | | |
| ΦNΞ 03698 | SAV1939 | 94 | | | |
| ΦNΞ 09316 | SAV1941 | 123 | | | |
| ΦNΞ 02996 | SAV1945 | 88 | | | |
| ΦNΞ 03552 | SAV1948 | 89 | | | |
| ΦNΞ 04335 | SAV1951 | 100 | | | |
| ΦNΞ 12961 | SAV1952 | 100 | | | |
| ΦNΞ 04092 | SAV1953 | 118 | | | |
| ΦNΞ 02902 | SAV1953 | 125 | | | |
| ΦNΞ 01647 | SAV1954 | 89 | | | |
| ΦNΞ 00355 | SAV1955 | 85 | | | |
| ΦNΞ 04496 | SAV1957 | 118 | | | |
| ΦNΞ 00548 | SAV1958 | 70 | | | |
| ΦNΞ 05637 | SAV1959 | 105 | | | |
| ΦNΞ 11198 | SAV1960 | 109 | | | |
| ΦNΞ 12540 | SAV1962 | 57 | 96 | | |
| ΦNΞ 11765 | SAV1964 | 118 | | | |
| ΦNΞ 06327 | SAV1965 | 50 | 41.4 | | |
| ΦNΞ 04147 | SAV1966 | 58 | 100 | | |
| ΦNΞ 03672 | SAV1967 | 65 | | | |
| ΦNΞ 03058 | SAV1968 | 44 | 80 | 87 | 98 |
| ΦNΞ 00613 | SAV1969 | 85 | | | |
| ΦNΞ 00311 | SAV1970 | 100 | | | |
| ΦNΞ 03622 | SAV1974 | 94 | | | |
| ΦNΞ 06446 | SAV1976 | 169 | | | |
| ΦNΞ 05162 | SAV1977 | 95 | | | |
| ΦNΞ 02679 | SAV1978 | 25 | 45 | 23 | 15 |
| ΦNΞ 11089 | SAV1980 | 109 | | | |
| ΦNΞ 04158 | SAV1980 | 37 | 80 | 88 | 97 |
| ΦNΞ 04765 | SAV1980 | 100 | | | |
| ΦNΞ 00050 | SAV1981 | 81 | | | |
| ΦNΞ 02109 | SAV1982 | 105 | | | |
| ΦNΞ 05258 | SAV1983 | 95 | | | |
| ΦNΞ 12674 | SAV1984 | 73 | 96 | | |
| ΦNΞ 03200 | SAV1986 | 38 | 60 | 83 | 28 |
| ΦNΞ 06697 | SAV1991 | 163 | | | |
| ΦNΞ 02683 | SAV1992 | 125 | | 48 | |
| ΦNΞ 01459 | SAV1992 | 98 | | | |
| ΦNΞ 03814 | SAV1994 | 75 | | | |
| ΦNΞ 09825 | SAV2001 | 105 | | | |
| ΦNΞ 09742 | SAV2002 | 105 | | | |
| ΦNΞ 04193 | SAV2003 | 53 | 100 | | |
| ΦNΞ 03288 | SAV2004 | 74 | | | |
| ΦNΞ 11331 | SAV2005 | 114 | | | |
| ΦNΞ 09440 | SAV2006 | 91 | | | |
| ΦNΞ 03441 | SAV2007 | 95 | | | |
| ΦNΞ 12603 | SAV2031 | 23 | 104 | | |
| ΦNΞ 01971 | SAV2034 | 105 | | | |
| ΦNΞ 04484 | SAV2034 | 65 | | | |
| ΦNΞ 04055 | SAV2036 | 65 | 30 | | |
| ΦNΞ 05384 | SAV2038 | 89 | 95 | | |
| ΦNΞ 03938 | SAV2039 | 100 | | | 63 |
| ΦNΞ 03962 | SAV2040 | 100 | | | |
| ΦNΞ 03868 | SAV2041 | 30 | 35 | | 100 |
| ΦNΞ 05101 | SAV2042 | 100 | | | |
| ΦNΞ 04946 | SAV2043 | 85 | 55 | 62 | 95 |
| ΦNΞ 04413 | SAV2045 | 118 | | | |
| ΦNΞ 04397 | SAV2046 | 118 | | | |
| ΦNΞ 05103 | SAV2048 | 95 | | | |
| ΦNΞ 00752 | SAV2053 | 50 | 100 | | |
| ΦNΞ 10056 | SAV2055 | 105 | | | |
| ΦNΞ 03592 | SAV2058 | 68 | | | |
| ΦNΞ 11921 | SAV2059 | 63 | 100 | | |
| ΦNΞ 06357 | SAV2060 | 92 | | | |
| ΦNΞ 04802 | SAV2060* | 95 | | | |
| ΦNΞ 00820 | SAV2061 | 56 | 95 | | |
| ΦNΞ 00789 | SAV2063 | 75 | | | |
| ΦNΞ 03805 | SAV2063 | 85 | | | |
| ΦNΞ 04540 | SAV2064 | 94 | | | |
| ΦNΞ 06056 | SAV2066 | 75 | 69 | | |
| ΦNΞ 00527 | SAV2067 | 60 | 95 | | |
| ΦNΞ 00556 | SAV2068 | 40 | 70 | 88 | 87 |
| ΦNΞ 09718 | SAV2069 | 105 | | | |
| ΦNΞ 13708 | SAV2070 | 22 | | | |
| ΦNΞ 04461 | SAV2073 | 112 | | | |
| ΦNΞ 03192 | SAV2074 | 113 | | | |
| ΦNΞ 09662 | SAV2075 | 105 | | | |
| ΦNΞ 03183 | SAV2077 | 94 | | | |
| ΦNΞ 02877 | SAV2078 | 125 | | | |
| ΦNΞ 00593 | SAV2078 | 100 | | | |
| ΦNΞ 04094 | SAV2079 | 100 | | | |
| ΦNΞ 12590 | SAV2081 | 37 | 46 | | |
| ΦNΞ 02828 | SAV2087 | 113 | | | |
| ΦNΞ 03083 | SAV2088 | 75 | | | |
| ΦNΞ 00196 | SAV2088 | 90 | | | |
| ΦNΞ 10618 | SAV2091 | 86 | | | |
| ΦNΞ 12882 | SAV2093 | 93 | | | |
| ΦNΞ 05639 | SAV2093 | 105 | | | |
| ΦNΞ 00426 | SAV2094 | 60 | 100 | | |
| ΦNΞ 05459 | SAV2096 | 105 | | | |
| ΦNΞ 02126 | SAV2101 | 105 | | | |
| ΦNΞ 02208 | SAV2104 | 74 | 100 | | |
| ΦNΞ 00209 | SAV2111 | 45 | 100 | | |
| ΦNΞ 03277 | SAV2112 | 89 | | | |
| ΦNΞ 09033 | SAV2113 | 24 | 96 | | |
| ΦNΞ 11642 | SAV2114 | 67 | 66 | | 98 |
| ΦNΞ 03191 | SAV2115 | 100 | | | |
| ΦNΞ 03531 | SAV2117 | 16 | 35 | 55 | 92 |
| ΦNΞ 01625 | SAV2121 | 63 | 100 | | |
| ΦNΞ 03903 | SAV2122 | 75 | 95 | | |
| ΦNΞ 13125 | SAV2123 | 4 | 75 | | |
| ΦNΞ 04714 | SAV2124 | 100 | | | |
| ΦNΞ 00608 | SAV2129 | 80 | | | |
| ΦNΞ 09408 | SAV2131 | 50 | 82 | | 98 |
| ΦNΞ 11140 | SAV2132 | 123 | | | |
| ΦNΞ 01579 | SAV2133 | 73 | | | |
| ΦNΞ 03816 | SAV2133 | 90 | | | |
| ΦNΞ 15294 | SAV2134 | 73 | 104 | | |
| ΦNΞ 11538 | SAV2135 | 54 | 69 | | 98 |
| ΦNΞ 04729 | SAV2136 | 100 | | | |
| ΦNΞ 04697 | SAV2137 | 100 | | | |
| ΦNΞ 00644 | SAV2138 | 80 | | | |
| ΦNΞ 13004 | SAV2142 | 100 | | | |
| ΦNΞ 00297 | SAV2143 | 85 | | | |
| ΦNΞ 01875 | SAV2144 | 100 | | | |
| ΦNΞ 13492 | SAV2145 | 102 | | | |
| ΦNΞ 00173 | SAV2146 | 85 | | | |
| ΦNΞ 04069 | SAV2147 | 106 | | | |
| ΦNΞ 12714 | SAV2152 | 90 | | | |
| ΦNΞ 09777 | SAV2153 | 95 | 100 | | |
| ΦNΞ 01551 | SAV2156 | 85 | | | |
| ΦNΞ 04123 | SAV2157 | 89 | | | |
| ΦNΞ 12774 | SAV2158 | 50 | 96 | | |
| ΦNΞ 05401 | SAV2159 | 105 | | | |
| ΦNΞ 04909 | SAV2160 | 95 | | | |
| ΦNΞ 09330 | SAV2161 | 27 | 18 | | |
| ΦNΞ 13821 | SAV2162 | 102 | | | |
| ΦNΞ 09246 | SAV2164 | 76 | 63.6 | 57.9 | |
| ΦNΞ 01617 | SAV2166 | 105 | | | |
| ΦNΞ 03603 | SAV2167 | 12 | 65 | 72 | 67 |
| ΦNΞ 01945 | SAV2168 | 84 | | | |
| ΦNΞ 03806 | SAV2169 | 75 | 90 | | |
| ΦNΞ 04933 | SAV2170 | 95 | | | 105 |
| ΦNΞ 03929 | SAV2171 | 85 | | | |
| ΦNΞ 04558 | SAV2172 | 118 | | | |
| ΦNΞ 00414 | SAV2174 | 85 | | | |
| ΦNΞ 03244 | SAV2174 | 100 | | | |
| ΦNΞ 04180 | SAV2175 | 53 | 75 | 95 | 98 |
| ΦNΞ 05526 | SAV2176 | 105 | | | |
| ΦNΞ 06699 | SAV2177 | 150 | | | |
| ΦNΞ 00049 | SAV2178 | 75 | | | |
| ΦNΞ 12559 | SAV2179 | 90 | | | |
| ΦNΞ 02896 | SAV2180 | 88 | 85 | | 100 |
| ΦNΞ 11381 | SAV2181 | 127 | | | |
| ΦNΞ 11306 | SAV2184 | 100 | | | |
| ΦNΞ 09843 | SAV2185 | 105 | | | |
| ΦNΞ 00615 | SAV2187 | 85 | | | |
| ΦNΞ 04401 | SAV2188 | 112 | | | |
| ΦNΞ 04314 | SAV2189 | 32 | 75 | 64 | 98 |
| ΦNΞ 05243 | SAV2189 | 90 | | | |
| ΦNΞ 06472 | SAV2190 | 175 | | | |
| ΦNΞ 06631 | SAV2191* | 163 | | | |
| ΦNΞ 03563 | SAV2192 | 74 | | | |
| ΦNΞ 02796 | SAV2193 | 119 | | | |
| ΦNΞ 00103 | SAV2194 | 90 | | | |
| ΦNΞ 03251 | SAV2194 | 89 | | | |
| ΦNΞ 00445 | SAV2197 | 55 | 100 | | |
| ΦNΞ 09119 | SAV2200 | 31 | 89 | | 100 |
| ΦNΞ 15274 | SAV2201 | 87 | | | |
| ΦNΞ 03517 | SAV2202 | 74 | | | |
| ΦNΞ 12117 | SAV2203 | 79 | | | |
| ΦNΞ 04489 | SAV2204 | 76 | | | |
| ΦNΞ 02906 | SAV2204 | 119 | | | |
| ΦNΞ 08826 | SAV2206 | 94 | | | |
| ΦNΞ 03959 | SAV2207 | 100 | | | |
| ΦNΞ 06362 | SAV2219 | 150 | | | |
| ΦNΞ 05576 | SAV2252 | 105 | | | |
| ΦNΞ 05438 | SAV2253 | 105 | | | |
| ΦNΞ 04663 | SAV2254 | 118 | | | |
| ΦNΞ 04767 | SAV2254 | 100 | | | |
| ΦNΞ 03598 | SAV2255 | 63 | 95 | | |
| ΦNΞ 01500 | SAV2256 | 91 | | | |
| ΦNΞ 01901 | SAV2258 | 89 | | | |
| ΦNΞ 00436 | SAV2261 | 100 | | | |
| ΦNΞ 03285 | SAV2261 | 95 | | | |
| ΦNΞ 10966 | SAV2265 | 109 | | | 100 |
| ΦNΞ 03874 | SAV2266 | 85 | | | |
| ΦNΞ 05250 | SAV2268 | 100 | | | |
| ΦNΞ 04699 | SAV2269 | 100 | | | |
| ΦNΞ 03509 | SAV2270 | 105 | | | |
| ΦNΞ 01567 | SAV2271 | 116 | | | |
| ΦNΞ 03209 | SAV2271 | 89 | | | |
| ΦNΞ 11375 | SAV2273 | 109 | | | |
| ΦNΞ 00662 | SAV2273 | 65 | | | |
| ΦNΞ 03257 | SAV2274 | 95 | | | |
| ΦNΞ 12467 | SAV2276 | 93 | | | |
| ΦNΞ 04648 | SAV2277 | 118 | | | |
| ΦNΞ 02985 | SAV2279 | 100 | | | |
| ΦNΞ 04839 | SAV2280 | 95 | | | |
| ΦNΞ 03843 | SAV2281 | 80 | | | |
| ΦNΞ 04375 | SAV2283 | 76 | | | |
| ΦNΞ 03239 | SAV2283 | 105 | | | |
| ΦNΞ 13637 | SAV2284 | 88 | 100 | | |
| ΦNΞ 03275 | SAV2285 | 79 | | | |
| ΦNΞ 03011 | SAV2287 | 63 | 90 | | |
| ΦNΞ 05068 | SAV2290 | 95 | | | |
| ΦNΞ 09184 | SAV2292 | 82 | 96 | | |
| ΦNΞ 01475 | SAV2293 | 110 | | | |
| ΦNΞ 12725 | SAV2294 | 93 | | | |
| ΦNΞ 03045 | SAV2297 | 63 | 80 | 55 | 100 |
| ΦNΞ 03230 | SAV2298 | 89 | | | |
| ΦNΞ 09996 | SAV2300 | 105 | | | |
| ΦNΞ 02916 | SAV2301 | 75 | 90 | | |
| ΦNΞ 11716 | SAV2302 | 118 | | | |
| ΦNΞ 05455 | SAV2304 | 105 | | | |
| ΦNΞ 11098 | SAV2305 | 127 | | | |
| ΦNΞ 03759 | SAV2306 | 94 | | | |
| ΦNΞ 11146 | SAV2306 | 123 | | | |
| ΦNΞ 09134 | SAV2307 | 86 | 86 | | 98 |
| ΦNΞ 04836 | SAV2309 | 100 | | | |
| ΦNΞ 03707 | SAV2310 | 112 | 20 | 43 | 97 |
| ΦNΞ 03164 | SAV2311 | 69 | 100 | | |
| ΦNΞ 04299 | SAV2313 | 105 | | | |
| ΦNΞ 03658 | SAV2313 | 100 | | | |
| ΦNΞ 02969 | SAV2314 | 119 | | | |
| ΦNΞ 05372 | SAV2315 | 100 | | | |
| ΦNΞ 02289 | SAV2315 | 74 | 100 | | |
| ΦNΞ 01488 | SAV2316 | 116 | | | |
| ΦNΞ 10856 | SAV2317 | 91 | | | |
| ΦNΞ 04077 | SAV2318 | 94 | | | |
| ΦNΞ 12860 | SAV2320 | 53 | 104 | | |
| ΦNΞ 03233 | SAV2321 | 105 | | | |
| ΦNΞ 12342 | SAV2322 | 57 | 93 | | |
| ΦNΞ 09671 | SAV2323 | 100 | 100 | | |
| ΦNΞ 12551 | SAV2324 | 83 | 100 | | |
| ΦNΞ 00733 | SAV2326 | 50 | 100 | | |
| ΦNΞ 04482 | SAV2327 | 59 | 85 | 55 | 100 |
| ΦNΞ 08886 | SAV2328 | 73 | 107 | | |
| ΦNΞ 01836 | SAV2329 | 105 | | | |
| ΦNΞ 04251 | SAV2330 | 100 | | | |
| ΦNΞ 03839 | SAV2331 | 95 | | | |
| ΦNΞ 11894 | SAV2332 | 88 | | | |
| ΦNΞ 09848 | SAV2333 | 105 | | | |
| ΦNΞ 03723 | SAV2334 | 71 | | | |
| ΦNΞ 12843 | SAV2335 | 100 | | | |
| ΦNΞ 01722 | SAV2337 | 84 | | | |
| ΦNΞ 04091 | SAV2338 | 112 | | | |
| ΦNΞ 11404 | SAV2339 | 132 | | | |
| ΦNΞ 04934 | SAV2340 | 45 | 80 | 58 | 98 |
| ΦNΞ 06075 | SAV2341 | 128 | | | |
| ΦNΞ 01529 | SAV2342 | 61 | 95 | | |
| ΦNΞ 03262 | SAV2344 | 47 | 95 | | |
| ΦNΞ 00352 | SAV2345 | 55 | 90 | 100 | |
| ΦNΞ 00096 | SAV2347 | 80 | | | |
| ΦNΞ 06759 | SAV2348 | 73 | 82, 77 | | 100 |
| ΦNΞ 11256 | SAV2350 | 109 | | | |
| ΦNΞ 03560 | SAV2352 | 100 | | | |
| ΦNΞ 05190 | SAV2354 | 95 | | | |
| ΦNΞ 02193 | SAV2355 | 89 | | | |
| ΦNΞ 00737 | SAV2356 | 45 | | | |
| ΦNΞ 01835 | SAV2356 | 11 | 75 | 28 | |
| ΦNΞ 05293 | SAV2358 | 95 | | | |
| ΦNΞ 03177 | SAV2359 | 113 | | | |
| ΦNΞ 02861 | SAV2360 | 125 | | | |
| ΦNΞ 08752 | SAV2360 | 76 | 100 | | |
| ΦNΞ 02823 | SAV2361 | 113 | | | |
| ΦNΞ 01562 | SAV2362 | 116 | | | |
| ΦNΞ 08907 | SAV2363 | 82 | 100 | | |
| ΦNΞ 00141 | SAV2365 | 60 | 90 | 98 | |
| ΦNΞ 03728 | SAV2366 | 41 | 95 | | |
| ΦNΞ 00287 | SAV2367 | 80 | | | |
| ΦNΞ 02684 | SAV2368 | 125 | | | |
| ΦNΞ 09868 | SAV2369 | 105 | | | |
| ΦNΞ 03958 | SAV2370 | 95 | | | |
| ΦNΞ 04025 | SAV2371 | 65 | 90 | | |
| ΦNΞ 03496 | SAV2372 | 100 | | | |
| ΦNΞ 00682 | SAV2373 | 85 | | | |
| ΦNΞ 01600 | SAV2374 | 110 | | | |
| ΦNΞ 02788 | SAV2374 | 125 | | | |
| ΦNΞ 07192 | SAV2375 | 90 | | | |
| ΦNΞ 04551 | SAV2376 | 53 | 100 | | |
| ΦNΞ 01749 | SAV2377 | 95 | | | |
| ΦNΞ 08917 | SAV2378 | 92 | | | |
| ΦNΞ 09727 | SAV2381 | 100 | 95 | | |
| ΦNΞ 09162 | SAV2382 | 102 | | | |
| ΦNΞ 03890 | SAV2383 | 95 | | | |
| ΦNΞ 09969 | SAV2384 | 105 | | | |
| ΦNΞ 00203 | SAV2385 | 95 | | | |
| ΦNΞ 02310 | SAV2386 | 84 | | | |
| ΦNΞ 11343 | SAV2388 | 118 | | | |
| ΦNΞ 01640 | SAV2389 | 105 | | | |
| ΦNΞ 13220 | SAV2390 | 100 | | | |
| ΦNΞ 00428 | SAV2392 | 100 | | | |
| ΦNΞ 10970 | SAV2393 | 136 | | | |
| ΦNΞ 00117 | SAV2394 | 100 | | | |
| ΦNΞ 03599 | SAV2395 | 47 | 95 | | |
| ΦNΞ 04939 | SAV2396 | 90 | | | |
| ΦNΞ 01710 | SAV2397 | 16 | 95 | | |
| ΦNΞ 05110 | SAV2397 | 95 | | | |
| ΦNΞ 12701 | SAV2398* | 73 | 79 | | 98 |
| ΦNΞ 05367 | SAV2399 | 100 | | | |
| ΦNΞ 09981 | SAV2399 | 105 | | | |
| ΦNΞ 00448 | SAV2400 | 60 | 100 | | |
| ΦNΞ 02449 | SAV2401 | 105 | | | |
| ΦNΞ 05127 | SAV2404 | 100 | | | |
| ΦNΞ 00423 | SAV2405 | 75 | | | |
| ΦNΞ 05619 | SAV2406 | 100 | | | |
| ΦNΞ 00671 | SAV2409 | 75 | | | |
| ΦNΞ 10967 | SAV2410 | 127 | | | |
| ΦNΞ 02665 | SAV2411 | 105 | | | |
| ΦNΞ 05574 | SAV2412 | 105 | | | |
| ΦNΞ 10833 | SAV2414 | 76 | 36.4 | 19.3 | |
| ΦNΞ 03616 | SAV2415 | 88 | | | |
| ΦNΞ 01732 | SAV2416 | 95 | | | |
| ΦNΞ 01483 | SAV2417 | 116 | | | |
| ΦNΞ 03151 | SAV2418 | 88 | | | |
| ΦNΞ 00267 | SAV2418 | 75 | | | |
| ΦNΞ 05500 | SAV2420 | 105 | | | |
| ΦNΞ 11423 | SAV2421 | 73 | | | |
| ΦNΞ 09907 | SAV2422 | 105 | | | |
| ΦNΞ 04521 | SAV2424 | 106 | | | |
| ΦNΞ 00654 | SAV2425 | 75 | | | |
| ΦNΞ 12770 | SAV2426 | 60 | 96 | | |
| ΦNΞ 09971 | SAV2427 | 105 | | | |
| ΦNΞ 05360 | SAV2428 | 100 | | | |
| ΦNΞ 12818 | SAV2429 | 40 | 86 | | |
| ΦNΞ 11692 | SAV2432 | 29 | 97 | | |
| ΦNΞ 01542 | SAV2433 | 98 | | | |
| ΦNΞ 04828 | SAV2434 | 90 | | | |
| ΦNΞ 03511 | SAV2436 | 100 | | | |
| ΦNΞ 00312 | SAV2436 | 95 | | | |
| ΦNΞ 06150 | SAV2436 | 128 | | | |
| ΦNΞ 02428 | SAV2437 | 105 | | | |
| ΦNΞ 03152 | SAV2438 | 106 | | | |
| ΦNΞ 04415 | SAV2439 | 94 | | | |
| ΦNΞ 10025 | SAV2440 | 105 | | | |
| ΦNΞ 04529 | SAV2441 | 118 | | | |
| ΦNΞ 11498 | SAV2442 | 67 | 79 | | 96 |
| ΦNΞ 05078 | SAV2443 | 100 | | | |
| ΦNΞ 03490 | SAV2444 | 95 | | | |
| ΦNΞ 04878 | SAV2445 | 100 | | | |
| ΦNΞ 04718 | SAV2446 | 100 | | | |
| ΦNΞ 14285 | SAV2448 | 100 | | | |
| ΦNΞ 04078 | SAV2449 | 100 | | | |
| ΦNΞ 04825 | SAV2450 | 80 | 100 | | |
| ΦNΞ 08781 | SAV2451 | 76 | 105 | | |
| ΦNΞ 06100 | SAV2452 | 78 | 100 | | |
| ΦNΞ 04174 | SAV2453 | 74 | | | |
| ΦNΞ 00440 | SAV2454 | 50 | 100 | | |
| ΦNΞ 05359 | SAV2455 | 100 | | | |
| ΦNΞ 05524 | SAV2458 | 105 | | | |
| ΦNΞ 04845 | SAV2459 | 90 | | | |
| ΦNΞ 09040 | SAV2460 | 93 | | | |
| ΦNΞ 03065 | SAV2462 | 44 | 55 | 88 | 97 |
| ΦNΞ 05105 | SAV2463 | 100 | | | |
| ΦNΞ 03694 | SAV2464 | 76 | | | |
| ΦNΞ 04010 | SAV2466 | 60 | 90 | | |
| ΦNΞ 05145 | SAV2467 | 95 | | | |
| ΦNΞ 04787 | SAV2468 | 100 | | | |
| ΦNΞ 03991 | SAV2469 | 100 | | | |
| ΦNΞ 04552 | SAV2469 | 118 | | | |
| ΦNΞ 03928 | SAV2470 | 90 | | | |
| ΦNΞ 04362 | SAV2473 | 112 | | | |
| ΦNΞ 00313 | SAV2476 | 100 | | | |
| ΦNΞ 05047 | SAV2477 | 90 | | | |
| ΦNΞ 03921 | SAV2478 | 95 | | | |
| ΦNΞ 02065 | SAV2479 | 84 | | | |
| ΦNΞ 06614 | SAV2480 | 163 | | | |
| ΦNΞ 00282 | SAV2481 | 80 | | | |
| ΦNΞ 11149 | SAV2483 | 27 | 90 | | |
| ΦNΞ 09762 | SAV2484 | 105 | | | |
| ΦNΞ 01846 | SAV2487 | 89 | | | |
| ΦNΞ 00390 | SAV2488 | 30 | 95 | | |
| ΦNΞ 03411 | SAV2488 | 95 | | | |
| ΦNΞ 00306 | SAV2489 | 100 | | | |
| ΦNΞ 02020 | SAV2490 | 95 | | | |
| ΦNΞ 03705 | SAV2492 | 41 | 95 | | |
| ΦNΞ 10624 | SAV2493* | 93 | | | |
| ΦNΞ 11084 | SAV2494 | 109 | | | |
| ΦNΞ 04326 | SAV2495 | 100 | | | |
| ΦNΞ 12431 | SAV2497 | 93 | | | |
| ΦNΞ 03242 | SAV2498 | 100 | | | |
| ΦNΞ 01952 | SAV2500 | 89 | | | |
| ΦNΞ 02964 | SAV2501 | 113 | | | |
| ΦNΞ 05396 | SAV2502 | 105 | | | |
| ΦNΞ 02646 | SAV2503 | 105 | | | |
| ΦNΞ 05115 | SAV2505 | 95 | | | |
| ΦNΞ 00576 | SAV2506 | 80 | | | |
| ΦNΞ 02437 | SAV2507 | 105 | | | |
| ΦNΞ 02984 | SAV2508 | 125 | | | |
| ΦNΞ 05634 | SAV2509 | 100 | | | |
| ΦNΞ 10441 | SAV2510 | 100 | | | |
| ΦNΞ 04803 | SAV2511 | 95 | | | |
| ΦNΞ 05605 | SAV2512 | 105 | | | |
| ΦNΞ 00223 | SAV2513 | 95 | | | |
| ΦNΞ 10052 | SAV2515 | 105 | | | |
| ΦNΞ 01975 | SAV2516 | 11 | 5 | | 91 |
| ΦNΞ 03402 | SAV2517 | 84 | | | |
| ΦNΞ 05603 | SAV2518 | 105 | | | |
| ΦNΞ 04104 | SAV2519 | 112 | | | |
| ΦNΞ 02392 | SAV2520 | 105 | | | |
| ΦNΞ 13298 | SAV2521 | 82 | 104 | | |
| ΦNΞ 10416 | SAV2522 | 83 | | | |
| ΦNΞ 11429 | SAV2523 | 83 | | | |
| ΦNΞ 03861 | SAV2525 | 100 | | | |
| ΦNΞ 09879 | SAV2526 | 100 | 100 | | |
| ΦNΞ 04500 | SAV2526 | 118 | | | |
| ΦNΞ 00284 | SAV2527 | 100 | | | |
| ΦNΞ 03486 | SAV2528 | 100 | | | 96 |
| ΦNΞ 04089 | SAV2529 | 106 | | | |
| ΦNΞ 08659 | SAV2530 | 83 | | | |
| ΦNΞ 03188 | SAV2531 | 88 | | | |
| ΦNΞ 05330 | SAV2532 | 100 | | | |
| ΦNΞ 10747 | SAV2534 | 97 | | | |
| ΦNΞ 00187 | SAV2535 | 80 | | | 105 |
| ΦNΞ 12724 | SAV2537 | 90 | | | |
| ΦNΞ 00373 | SAV2538 | 70 | | | |
| ΦNΞ 04017 | SAV2539 | 90 | | | |
| ΦNΞ 08702 | SAV2540 | 86 | | | |
| ΦNΞ 12224 | SAV2541 | 42 | 100 | | |
| ΦNΞ 00643 | SAV2542 | 80 | | | |
| ΦNΞ 11917 | SAV2543 | 100 | | | |
| ΦNΞ 06492 | SAV2544 | 166 | | | |
| ΦNΞ 14634 | SAV2546 | 63 | 61 | | |
| ΦNΞ 00319 | SAV2548 | 80 | | | |
| ΦNΞ 04186 | SAV2549 | 37 | 75 | 83 | 100 |
| ΦNΞ 04437 | SAV2550 | 76 | | | |
| ΦNΞ 09781 | SAV2550 | 105 | | | |
| ΦNΞ 04953 | SAV2551 | 95 | | | |
| ΦNΞ 01546 | SAV2552 | 91 | | | |
| ΦNΞ 03159 | SAV2552 | 6 | 20 | 50 | 98 |
| ΦNΞ 00458 | SAV2554 | 35 | 100 | | |
| ΦNΞ 02145 | SAV2555 | 105 | | | |
| ΦNΞ 04387 | SAV2557 | 82 | | | |
| ΦNΞ 11762 | SAV2558 | 114 | | | |
| ΦNΞ 01477 | SAV2560 | 61 | 100 | | |
| ΦNΞ 00022 | SAV2561 | 100 | | | |
| ΦNΞ 03594 | SAV2561 | 89 | | | |
| ΦNΞ 01602 | SAV2562 | 85 | | | |
| ΦNΞ 02946 | SAV2563 | 94 | | | |
| ΦNΞ 09114 | SAV2564 | 69 | 50 | 33.3 | |
| ΦNΞ 04150 | SAV2565 | 105 | | | |
| ΦNΞ 00516 | SAV2566 | 70 | | | |
| ΦNΞ 03895 | SAV2566 | 100 | | | |
| ΦNΞ 00792 | SAV2567 | 45 | 25 | 25 | 53 |
| ΦNΞ 02863 | SAV2567 | 75 | 55 | | 48 |
| ΦNΞ 02695 | SAV2568 | 125 | | | |
| ΦNΞ 00237 | SAV2569 | 90 | | | |
| ΦNΞ 06710 | SAV2570 | 150 | | | |
| ΦNΞ 08644 | SAV2571 | 94 | | | |
| ΦNΞ 03781 | SAV2572 | 100 | | | |
| ΦNΞ 10619 | SAV2573 | 93 | | | |
| ΦNΞ 00838 | SAV2576 | 82 | | | |
| ΦNΞ 04774 | SAV2576 | 100 | | | |
| ΦNΞ 03283 | SAV2578 | 79 | | | |
| ΦNΞ 11432 | SAV2579 | 46 | 90 | | |
| ΦNΞ 04358 | SAV2580 | 106 | | | |
| ΦNΞ 14009 | SAV2581 | 90 | | | |
| ΦNΞ 05189 | SAV2582 | 85 | | | |
| ΦNΞ 11809 | SAV2583 | 83 | | | |
| ΦNΞ 00657 | SAV2584 | 65 | | | |
| ΦNΞ 02997 | SAV2584 | 81 | | | |
| ΦNΞ 00421 | SAV2585 | 60 | 100 | | |
| ΦNΞ 10636 | SAV2587 | 97 | | | |
| ΦNΞ 04914 | SAV2588 | 100 | | | |
| ΦNΞ 04893 | SAV2591 | 90 | | | |
| ΦNΞ 03919 | SAV2594 | 90 | | | |
| ΦNΞ 02296 | SAV2597 | 105 | | | |
| ΦNΞ 06644 | SAV2599 | 166 | | | |
| ΦNΞ 05452 | SAV2600 | 105 | | | |
| ΦNΞ 12720 | SAV2602 | 73 | 89 | | |
| ΦNΞ 13035 | SAV2603 | 100 | | | |
| ΦNΞ 00171 | SAV2604 | 95 | | | |
| ΦNΞ 02730 | SAV2604 | 119 | | | |
| ΦNΞ 04971 | SAV2605 | 100 | | | |
| ΦNΞ 12975 | SAV2606 | 100 | | | |
| ΦNΞ 00773 | SAV2607 | 80 | | | |
| ΦNΞ 11441 | SAV2608 | 117 | | | |
| ΦNΞ 04821 | SAV2609 | 95 | | | |
| ΦNΞ 08980 | SAV2610 | 71 | 93 | | |
| ΦNΞ 04344 | SAV2612 | 100 | | | |
| ΦNΞ 04535 | SAV2613 | 112 | | | |
| ΦNΞ 06718 | SAV2614 | 163 | | | |
| ΦNΞ 02034 | SAV2615 | 89 | | | |
| ΦNΞ 04459 | SAV2616 | 112 | | | |
| ΦNΞ 04385 | SAV2617 | 76 | | | |
| ΦNΞ 02850 | SAV2618 | 100 | | | |
| ΦNΞ 12167 | SAV2619 | 92 | 97 | | |
| ΦNΞ 13059 | SAV2620 | 100 | | | |
| ΦNΞ 02612 | SAV2621 | 105 | | | |
| ΦNΞ 04001 | SAV2622 | 90 | | | |
| ΦNΞ 03977 | SAV2622 | 100 | | | |
| ΦNΞ 03452 | SAV2623 | 100 | | | |
| ΦNΞ 05069 | SAV2624 | 95 | | | |
| ΦNΞ 05304 | SAV2625 | 75 | 90 | | |
| ΦNΞ 02121 | SAV2627 | 105 | | | |
| ΦNΞ 02802 | SAV2629 | 125 | | | |
| ΦNΞ 00826 | SAV2630 | 103 | | | 103 |
| ΦNΞ 13125 | SAV2630* | 4 | 75 | | 76 |
| ΦNΞ 13153 | SAV2630* | 98 | | | |
| ΦNΞ 05159 | SAV2631 | 100 | | | |
| ΦNΞ 04271 | SAV2632 | 68 | | | |
| ΦNΞ 05038 | SAV2633 | 100 | | | |
| ΦNΞ 01608 | SAV2634 | 110 | | | |
| ΦNΞ 04557 | SAV2635 | 112 | | | |
| ΦNΞ 00400 | SAV2636 | 95 | | | |
| ΦNΞ 09030 | SAV2637 | 80 | 79 | | 100 |
| ΦNΞ 08682 | SAV2638 | 65 | 100 | | 100 |
| ΦNΞ 13671 | SAV2639 | 86 | 104 | | |
| ΦNΞ 05231 | SAV2640 | 100 | | | |
| ΦNΞ 00382 | SAV2641 | 95 | | | |
| ΦNΞ 04717 | SAV2642 | 95 | | | |
| ΦNΞ 11406 | SAV2643 | 114 | | | |
| ΦNΞ 02659 | SAV2644 | 105 | | | |
| ΦNΞ 04073 | SAV2645 | 118 | | | |
| ΦNΞ 00505 | SAV2646 | 55 | 95 | | |
| ΦNΞ 09723 | SAV2646 | 105 | | | |
| ΦNΞ 01876 | SAV2647 | 95 | | | |
| ΦNΞ 03626 | SAV2647 | 100 | | | |
| ΦNΞ 00083 | SAV2648 | 85 | | | |
| ΦNΞ 04051 | SAV2649 | 106 | | | |
| ΦNΞ 00878 | SAV2650 | 51 | 95 | | |
| ΦNΞ 02762 | SAV2651 | 119 | | | |
| ΦNΞ 04305 | SAV2652 | 32 | 100 | | |
| ΦNΞ 02681 | SAV2652 | 125 | | | |
| ΦNΞ 03558 | SAV2652 | 95 | | | |
| ΦNΞ 01583 | SAV2653 | 37 | 100 | | |
| ΦNΞ 04676 | SAV2654 | 100 | | | 98 |
| ΦNΞ 04712 | SAV2654 | 95 | | | |
| ΦNΞ 06330 | SAV2655 | 98 | | | |
| ΦNΞ 11230 | SAV2658 | 105 | | | |
| ΦNΞ 11724 | SAV2659 | 95 | | | |
| ΦNΞ 04057 | SAV2660 | 94 | | | |
| ΦNΞ 02386 | SAV2660 | 89 | | | |
| ΦNΞ 03156 | SAV2661 | 25 | 70 | 87 | 98 |
| ΦNΞ 04689 | SAV2662 | 100 | | | |
| ΦNΞ 02030 | SAV2663 | 100 | | | |
| ΦNΞ 12143 | SAV2664 | 42 | 90 | | |
| ΦNΞ 09269 | SAV2665 | 93 | | | |
| ΦNΞ 04722 | SAV2666 | 100 | | | |
| ΦNΞ 04780 | SAV2666 | 100 | | | |
| ΦNΞ 01598 | SAV2667 | 91 | | | |
| ΦNΞ 03146 | SAV2668 | 81 | | | |
| ΦNΞ 05081 | SAV2669 | 65 | 90 | | |
| ΦNΞ 04872 | SAV2671 | 90 | | | |
| ΦNΞ 04758 | SAV2672 | 100 | | | |
| ΦNΞ 03963 | SAV2673 | 65 | 90 | | |
| ΦNΞ 03892 | SAV2674 | 95 | | | |
| ΦNΞ 09374 | SAV2674 | 82 | | | |
| ΦNΞ 02790 | SAV2675 | 125 | | | |
| ΦNΞ 00062 | SAV2676 | 56 | | | |
| ΦNΞ 02701 | SAV2676 | 125 | | | |
| ΦNΞ 00731 | SAV2677 | 70 | | | |
| ΦNΞ 00416 | SAV2678 | 80 | | | |
| ΦNΞ 02009 | SAV2678 | 89 | | | |
| ΦNΞ 02348 | SAV2679 | 100 | | | |
| ΦNΞ 00619 | SAV2680 | 90 | | | |
| ΦNΞ 01573 | SAV2681 | 110 | | | |
| ΦNΞ 12301 | SAV2683 | 67 | 79 | | |
| ΦNΞ 00726 | SAV2684 | 60 | 100 | | |
| ΦNΞ 01613 | SAV2685 | 84 | | | |
| ΦNΞ 00734 | SAV2686 | 60 | 95 | | |
| ΦNΞ 02732 | SAV2688 | 119 | | | |
| ΦNΞ 05335 | SAV2689 | 95 | | | |
| ΦNΞ 00709 | SAV2689 | 85 | | | |
| ΦNΞ 03122 | SAV2690 | 122 | | | |
| ΦNΞ 04754 | SAV2691 | 100 | | | |
| ΦNΞ 02837 | SAV2694 | 119 | | | |
| ΦNΞ 04759 | SAV2696 | 95 | | | |
| ΦNΞ 00295 | SAV2697 | 95 | | | |
| ΦNΞ 03556 | SAV2698 | 84 | | | |
| ΦNΞ 09337 | SAV2699 | 50 | 93 | | |
| ΦNΞ 02821 | SAV2700 | 125 | | | |
| ΦNΞ 09820 | SAV2700 | 100 | 100 | | |
| ΦNΞ 03823 | SAV2701 | 85 | | | |
| ΦNΞ 03436 | SAV2705 | 84 | | | |
| ΦNΞ 03664 | SAV2706 | 47 | 80 | 97 | |
| ΦNΞ 02441 | SAV2707 | 105 | | | |
| ΦNΞ 09932 | SAV2707 | 105 | | | |
| ΦNΞ 13476 | SAV2710 | 98 | | | |
| ΦNΞ 02246 | SAV2711 | 100 | | | |
| ΦNΞ 03570 | SAV2712 | 105 | | | |
| ΦNΞ 02773 | MW0055 | 113 | | | |
| ΦNΞ 02293 | MW0063 | 105 | | | |
| ΦNΞ 02272 | MW0064 | 105 | | | |
| ΦNΞ 05443 | MW0073 | 105 | | | |
| ΦNΞ 04871 | MW0098 | 85 | 95 | | |
| ΦNΞ 03653 | MW0120 | 65 | | | |
| ΦNΞ 02763 | MW0387 | 119 | | | |
| ΦNΞ 03693 | MW0398 | 41 | 100 | | |
| ΦNΞ 04808 | MW0554 | 100 | | | |
| ΦNΞ 02045 | MW0555 | 105 | | | |
| ΦNΞ 04799 | MW0558 | 100 | | | |
| ΦNΞ 04898 | MW0559 | 80 | 75 | 51 | |
| ΦNΞ 03457 | MW0562 | 100 | | | |
| ΦNΞ 03435 | MW0764 | 74 | | | |
| ΦNΞ 03828 | MW0765 | 85 | | | |
| ΦNΞ 05133 | MW0918 | 100 | | | |
| ΦNΞ 02413 | MW1201 | 105 | | | |
| ΦNΞ 10206 | MW1380 | 105 | | | |
| ΦNΞ 05098 | MW1442 | 100 | | | |
| ΦNΞ 03507 | MW1742 | 95 | | | |
| ΦNΞ 00599 | MW1743 | 105 | | | |
| ΦNΞ 02003 | MW1749 | 105 | | | |
| ΦNΞ 02041 | MW1760 | 105 | | | |
| ΦNΞ 03278 | MW1761 | 79 | | | |
| ΦNΞ 03627 | MW1762 | 47 | 75 | 97 | |
| ΦNΞ 05631 | MW1771 | 105 | | | |
| ΦNΞ 03866 | MW1947 | 95 | | | |
| ΦNΞ 02690 | MW2125 | 125 | | | |
| ΦNΞ 02436 | MW2197 | 105 | | | |
| ΦNΞ 03736 | MW2377 | 59 | 95 | | |
| ΦNΞ 03748 | MW2534 | 71 | | | |
| ΦNΞ 00038 | SA0877 | 118 | | | |
| ΦNΞ 00473 | SA1787 | 85 | | | |
| ΦNΞ 01711 | SA1802 | 16 | 95 | | |
| ΦNΞ 03466 | SA1806 | 105 | | | |
| ΦNΞ 05547 | SAS067 | 100 | | | |
| ΦNΞ 02052 | SA2074 | 105 | | | |

### EXAMPLE 2

### Creation of Transposon Element, Bursa aurealis

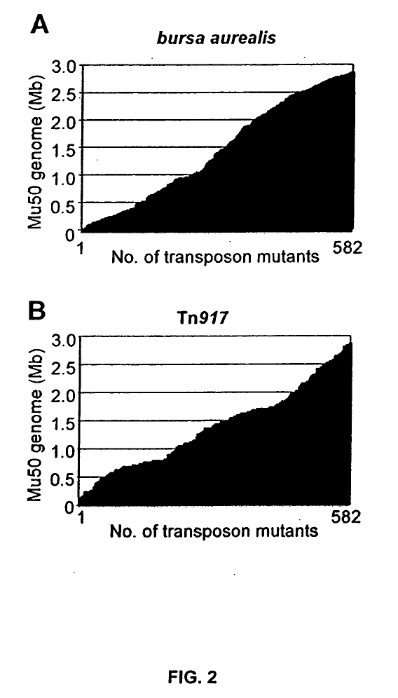

Bursa aurealis was derived from the Himar 1 (mariner) element; it encompasses the short inverted repeats of the hornfly transposon (Somerville et al., 2002; Robertson et al., 1995) as well as the ermC resistance marker (Trieu-Cuot et al., 1990) and a promotorless Aequorea victoria green fluorescent protein gene (FIG. 1). Insertion of bursa aurealis into genome sequences confers resistance to erythromycin and expression of gfp if the insertion occurred in the vicinity of promoter sequences. Bursa aurealis was cloned into the pTS2 vector with a temperature-sensitive S. aureus replicon and chloramphenicol resistance gene (Fitzgerald et al., 2000), thereby generating pBursa (FIG. 1). The Himar 1 transposase (Lampe et al., 1996) was cloned along with the xylose -repressor of pIK64 (Kullik et al., 1998) into plasmid pSPT246ts (Janzon et al., 1990), resulting in the transposase vector pFA545 (FIG. 1 top). Plasmids were transformed into S. aureus strain Newman by electroporation, and mutants were isolated by temperature shift to 43° C. and by streaking for erythromycin-resistant phenotype. Isolated mutants were stored frozen and the site of transposition for 10,325 isolates was examined by inverse PCR of bursa aurealis flanking sequences. Analysis of insertion sequences revealed that bursa aurealis generates TA duplications at the insertion sites and does not exhibit sequence preference in the S. aureus genome (FIG. 2).

### EXAMPLE 3

### Analysis of bursa aurealis and Tn917 Transposition Events

Bursa aurealis mutagenesis was compared to populations of transposon insertion mutants generated by Tn917. These Tn917 populations were created using plasmid pTV1 (Hartley et al., 1986), which carries Tn917—a transposon that has been the mobile genetic element of choice for insertional mutagenesis in gram-positive microbes and specifically in S. aureus strains (Mei et al., 1997; Schwan et al., 1998). After transformation of pTV1 and temperature-induced selection of transposon insertions, 960 S. aureus strain Newman mutants were generated with Tn917. Of 960 mutants, 585 mutants produced readable sequencing results and the Tn917 insertion site was determined using the same technique as described above for bursa aurealis. Insertion in non-coding DNA sequences of S. aureus occurred at a higher frequency for Tn917 (36% of all insertions as compared to 18% for bursa aurealis). Tn917 exhibited a strong bias for insertion in two regions on the staphylococcal chromosome centered at Mbp 0.75 and 1.7, respectively (FIG. 2). 587 insertions of Tn917 were located in 269 different genes, while the same number of bursa aurealis insertions hit 350 different genes. In addition, bursa aurealis insertions did not display the same bias for insertion at Mbp 0.75 and 1.7 as is reported here for Tn917 (FIG. 2).

### EXAMPLE 4

### Bursa aurealis Mutagenesis of S. aureus Newman

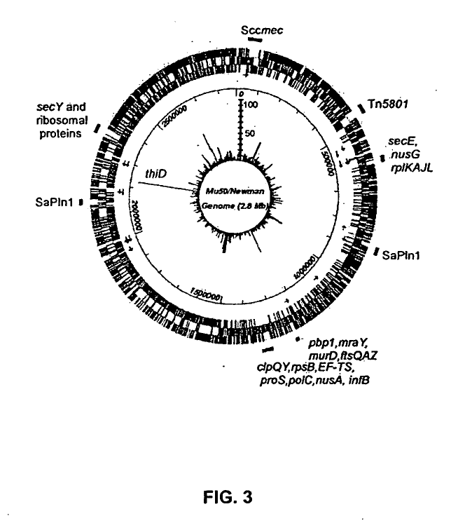

A total of 10,325 bursa aurealis isolates were generated, sequenced and the open reading frame (Orf) insertions mapped as outlined above (FIG. 3). Comparison of the data set with the genome sequence of S. aureus strain Mu50 indicated that 6,917 mutants carried bursa aurealis insertions within coding sequences, while 1,533 insertions occurred within intergenic or non-coding DNA sequences (ΦNΞ, Phoenix library, Table 3—located after Examples). The entire pBursa plasmid inserted at the S. aureus pre plasmid recombination site, yielding chloramphenicol, erythromycin double-resistant colonies that accounted for 7.6% of erythromycin-resistant colonies following mutagenesis. Together these experiments provided several insights. (i) The mutagenesis of the S. aureus chromosome with 10,325 bursa aurealis insertions was not saturated. Using the acquired data set for prediction, about 20,000 random insertion mutants would be required to achieve saturating mutagenesis. (ii) Based on the comparison of raw sequence data with the published genome sequence of S. aureus strain Mu50, it appears that S. aureus strain Newman encodes approximately 2,700 genes. Approximately 67% of all Orfs, 1,812 genes, have been disrupted by bursa aurealis insertion (FIG. 3). Table 3 summarizes all insertion mutants that have an identifiable BLAST hit in one of three completed staphylococcal genomes (Mu50, MW2, and N315). The prefixes indicate which genome sequence the Newman insertion mutant matches: SAV corresponds to Mu50, MW to MW2, and SA and SAS to N315. An asterisk in the column marked IGR indicates an insertion in an intergenic region, but within ten nucleotides of a neighboring gene.

### EXAMPLE 5

### S. aureus Strain Newman Genes

Previous work compared the contents of S. aureus genome sequences and pointed to genes that occur in some, but not in all, staphylococcal isolates (Kuroda et al., 2001; Baba et al., 2002). If one assumed that essential or housekeeping gene functions may be shared among staphylococci, some of the S. aureus Newman genes that are not found in other staphylococcal isolates may be uniquely responsible for animal virulence of strain Newman. For example, a comparison of the S. aureus MW2 genome sequence (a community-acquired MRSA strain) with the sequences of S. aureus strains N315 and Mu50 (hospital-acquired MRSA) revealed 94.7% and 94.8% sequence identities, respectively (N315 and Mu50 display 99.7% nucleotide match) (Baba et al., 2002). When analyzed with respect to the bursa aurealis pool of mutants, 234 insertions occurred in S. aureus strain Newman genes that did not display homology to other staphylococcal gene sequences and that may therefore represent mobile genetic elements or even virulence genes. FIG. 3 shows that several mobile gene elements of strain Mu50 display only limited or no homology to S. aureus Newman gene sequences.

### EXAMPLE 6

### S. aureus Virulence Genes Identified by Nematode Killing

Ausubel, Sifri, Calderwood and colleagues introduced the nematode Caenorhabditis elegans as a model host for the pathogenesis of S. aureus infections (Garsin et al., 2001; Sifri et al., 2003). When incubated on agar plates with either L4 larvae or adult worms, staphylococci enter the worm oral cavity, pass the food grinder and multiply in the C. elegans digestive tract. Colonization with S. aureus causes distension of the digestive tract and ultimately killing of infected worms over a period of 2-7 days (Sifri et al., 2003). Using this assay, Sifri and colleagues demonstrated that several known S. aureus virulence factors are also required for the killing of C. elegans (Sifri et al., 2003). Nematode killing was chosen as a first assay to identify virulence genes. 1,736 S. aureus Newman mutants with a bursa aurealis insertion were screened on agar plates for the ability to kill between 30-50 worms. Adult C. elegans proved to be more susceptible to S. aureus infection than larvae and were therefore used the former to screen the population of inserts. Both live and dead worms were enumerated under a dissecting microscope after 48 hours incubation at 25° C. on agar plates. Statistical significance was examined by Chi-square analysis and mutants with a p value below 0.1 were submitted to a second and third screening procedure with the same parameters. After three screening assays, 174 insertion mutants displayed reduced virulence and were analyzed further (p<0.05). To determine whether unrelated mutations caused the observed reduced virulence phenotypes, each strain was lysed with phage φ85 (Mazmanian et al., 2000) and transposon insertions were transduced into S. aureus wild-type strain Newman. The transduced bursa aurealis insertion sites were again mapped by inverse PCR and DNA sequencing. The transduced variants were then screened for defects in virulence using agar plate infections of either adult worms or L4 larvae. 71 mutants of the original 174 displayed reduced virulence in both assays (p<0.05) (Table 2), encompassing 41 genes with assigned function and 30 hypothetical open reading frames (Kuroda et al., 2001). Genes with assigned functions could be classified into three categories: metabolism, regulation, and extracellular functions.

TABLE 2
S. aureus strain Newman genes required for nematode killing

| | | C. elegans | | | |
|---|---|---|---|---|---|
| Phoenix | | killing (%) | | | |
| Mutant | Orf | Adult | L4 | Gene | Predicted Product |

Insertions in metabolic genes

| | | | | | |
|---|---|---|---|---|---|
| ΦNΞ05074 | SAV0185 | Ns | 64 | argD1 | ornithine aminotransferase |
| ΦNΞ05298 | SAV0248 | Ns | 75 | gutB | sorbitol dehydrogenase |
| ΦNΞ03871 | SAV0475 | 10 | 11 | agl | alpha-glucosidase |
| ΦNΞ01545 | SAV0588 | 5 | 25 | pta | phosphostransacetylase |
| ΦNΞ02038 | SAV0643 | 23 | 32 | abcA | ATP-binding cassette transporter A |
| ΦNΞ03711 | SAV0711 | 90 | 69 | ptpS | 6-pyruvoyl tetrahyrobiopterin synthase |
| | | | | | homologue |
| ΦNΞ10868 | SAV1094 | 22 | 0 | phdB | pyruvate dehydrogenase E1 component |
| ΦNΞ00656 | SAV1201 | Ns | 72 | pyrC | dihydroorotase |
| ΦNΞ00678 | SAV1202 | Ns | 77 | pyrAA | carbamoyl-phosphate synthase small chain |
| ΦNΞ02887 | SAV1203 | 88 | ns | pyrAB | carbamoyl-phosphate synthase large chain |
| ΦNΞ02904 | SAV1205 | Ns | 64 | pyrE | orotate phosphoribosyltransferase |
| ΦNΞ00263 | SAV1350 | 58 | 34 | citB | aconitase |
| ΦNΞ09110 | SAV1356 | Ns | 41 | alsT | sodium/alanine symporter |
| ΦNΞ10240 | SAV1370 | 72 | 11 | trpC | indole-3-glycerol phosphate synthase |
| ΦNΞ02026 | SAV1413 | Ns | 30 | odhA | oxoglutarate dehydrogenase |
| ΦNΞ10390 | SAV1518 | 90 | 1 | lpd | dihydrolipoamide dehydrogenase |
| ΦNΞ01982 | SAV1687 | 77 | 36 | gapB | glyceraldehyde 3-phosphoate |
| | | | | | dehydrogenase 2 |
| ΦNΞ05271 | SAV1694 | 76 | 52 | citC | isocitrate dehydrogenase |
| ΦNΞ11740 | SAV1765 | Ns | 77 | | lysophospholipase homologue |
| ΦNΞ01969 | SAV1791 | Ns | 77 | pckA | phosphoenolpyruvate carboxykinase |
| ΦNΞ03937 | SAV1851 | 63 | 30 | citG | fumarate hydratase |
| ΦNΞ04769 | SAV1918 | 82 | 80 | | pyrazinamidase/nicotinamidase homologue |
| ΦNΞ09246 | SAV2164 | Ns | 77 | arg | arginase |
| ΦNΞ01975 | SAV2516 | 26 | 23 | fbp | fructose-biphosphatase |
| ΦNΞ04818 | SAV0511 | 75 | 11 | ftsH | ATP-dependent Zn metallopeptidase |
| ΦNΞ13116 | SAV0768 | 31 | 2 | clpP | ATP-dependent Clp protease |
| ΦNΞ09483 | SAV0721 | 68 | 44 | recQ | probable DNA helicase |
| ΦNΞ11173 | SAV1227 | 84 | 10 | recG | ATP-dependent DNA helicase |

Insertions in regulatory genes

| | | | | | |
|---|---|---|---|---|---|
| ΦNΞ09725 | SAV0705 | Ns | 61 | saeS | sensor histidine kinase |
| ΦNΞ01594 | SAV0706 | Ns | 73 | saeR | response regulator |
| ΦNΞ12844 | SAV1491 | Ns | 57 | srrB | staphylococcal respiratory response protein |
| ΦNΞ01835 | SAV2356 | 55 | 57 | tcaA | TcaA protein |
| ΦNΞ06220 | SAV2355 | Ns | 64 | tcaB | TcaB protein |
| ΦNΞ11797 | SAV1362 | 87 | 68 | msrR | peptide methionine sulfoxide reductase |
| | | | | | regulator |
| ΦNΞ02575 | SAV2499 | Ns | 73 | sarH2 | sarA homologue |

Insertions in genes encoding extracellular factors

| | | | | | |
|---|---|---|---|---|---|
| ΦNΞ09823 | SAV0815 | 93 | 66 | nuc | staphylococcal nuclease |
| ΦNΞ02140 | SAV1163 | Ns | 57 | hla | aipha-hemolysin |
| ΦNΞ05448 | SAV0154 | 63 | 67 | cap5F | capsular polysaccharide synthesis enzyme |
| ΦNΞ03492 | SAV0158 | 71 | Ns | capJ | capsular polysaccharide synthesis enzyme |
| ΦNΞ01957 | SAV0161 | 76 | Ns | capM | capsular polysaccharide synthesis enzyme |
| ΦNΞ09330 | SAV2161 | 35 | 40 | glmM | phosphoglucosamine-mutase |

Insertions in unknown genes

| | | | | |
|---|---|---|---|---|
| ΦNΞ03224 | SAV0258 | 85 | Ns | similar to beta-glycosyltransferase |
| ΦNΞ01776 | SAV0709 | 78 | Ns | conserved hypothetical protein |
| ΦNΞ11117 | SAV0713 | Ns | 50 | hypothetical protein |
| ΦNΞ04880* | SAV0727* | Ns | 80 | similar to di-tripeptide ABC transporter |
| ΦNΞ04279 | SAV0841 | 10 | 51 | conserved hypothetical protein |
| ΦNΞ07262* | SAV0855* | 70 | 18 | hypothetical protein (phiMu50B) |
| ΦNΞ12478* | SAV0862* | Ns | 21 | hypothetical protein (phiMu50B) |
| ΦNΞ08772* | SAV0866* | Ns | 50 | hypothetical protein (phiMu50B) |
| ΦNΞ03004* | SAV0876* | 17 | 13 | hypothetical protein (phiMu50B) |
| ΦNΞ10876 | SAV0940 | Ns | 66 | conserved hypothetical protein |
| ΦNΞ05437 | SAV0944 | 25 | 11 | conserved hypothetical protein |
| ΦNΞ00126 | SAV1012 | 35 | 78 | hypothetical protein |
| ΦNΞ10949 | SAV1190 | Ns | 73 | conserved hypothetical protein |
| ΦNΞ12600 | SAV1221 | 26 | 53 | conserved hypothetical protein |
| ΦNΞ01851* | SAV1619* | 71 | 63 | similar to deoxyribonuclease |
| ΦNΞ01639 | SAV1677 | 48 | 63 | conserved hypothetical protein |
| ΦNΞ01819 | SAV1706 | Ns | 51 | conserved hypothetical protein |
| ΦNΞ09771 | SAV1725 | 75 | 20 | hypothetical protein |
| ΦNΞ04883* | SAV1926* | 30 | 11 | hypothetical protein |
| ΦNΞ11080* | SAV1927* | 20 | 3 | conserved hypothetical protein |
| ΦNΞ01449 | SAV1928 | 15 | 13 | hypothetical protein |
| ΦNΞ01508* | SAV1932* | Ns | 13 | hypothetical protein |
| ΦNΞ02679 | SAV1978 | 15 | 15 | hypothetical protein (phiMu50A) |
| ΦNΞ03200* | SAV1986* | 28 | 59 | hypothetical protein (phiMu50A) |
| ΦNΞ00556 | SAV2068 | 86 | Ns | hypothetical protein |
| ΦNΞ03531 | SAV2117 | Ns | 61 | similar to protoporphyrinogen oxidase HemK |
| ΦNΞ13125 | SAV2123 | 76 | Ns | conserved hypothetical protein |
| ΦNΞ36035 | SAV2167 | 66 | Ns | hypothetical protein |
| ΦNΞ02863 | SAV2175 | Ns | 65 | similar to ferrichrome ABC transporter |
| ΦNΞ04180* | SAV2567* | 51 | 48 | similar to acyltransferase |

Data are reported as % worm killing based on 100% killing by wild type Newman.
ΦNΞ mutants with underlined orfs displayed growth defects in TSB medium at 37° C.
ΦNΞ mutants marked with an asterisks (*) were tested in a mouse model of infection.
Ns, not significant.

### EXAMPLE 7

### S. aureus Mutants with Insertions in Metabolic Genes

About 70% of the bursa aurealis mutants with virulence defects carry insertions in genes responsible for energy metabolism or intermediary metabolism. This observation corroborates earlier work using IVET transposon screening techniques to define virulence defects in S. aureus, Salmonella species or other enteric pathogens (Lowe et al., 1998; Mahan et al., 1993). Further, enzymes of the S. aureus TCA cycle such as OdhB and CitB are required for S. aureus virulence in a murine model of bacteremia. CitB (aconitase) uses an iron sulfur cluster to catalyze the conversion of citrate to isocitrate (Kohler et al., 2003). Limited iron conditions affect the integrity of this iron sulfur cluster, leading to an arrest of the TCA cycle, accumulation of citrate and a switch in the activity of CitB from hydrolyase to RNA binding. In E. coli and B. subtilis, the regulatory RNA binding protein CitB is involved in the oxidative stress response. In S. aureus, CitB inactivation may affect the level of other factors such as RNAIII, the regulatory molecule that controls the output of the Agr-dependent quorum sensing system (Novick et al., 1993). Killing of C. elegans by S. aureus was markedly decreased in mutants interfering with other metabolic pathways such as urea cycle, pyrimidine biosynthesis, sugar and amino acid metabolism (Table 2). Nematode killing was also reduced for two mutants impaired in DNA helicase activity (recG and recQ). Bursa aurealis insertions of recA have thus far not been isolated. However, signature-tag mutagenesis previously identified recA as required for virulence in a murine bacteremia model (Mei et al., 1997). Together these results suggest that replication, recombination and thus functional maintenance of chromosomal DNA are important for virulence. Quality control of protein biogenesis may also be required for virulence. An ftsH mutation exhibited decreased virulence in the nematode killing assay. This observation is in agreement with ftsH mutants displaying attenuated virulence in a murine skin abscess model (Lithgow et al., 2004). ATP-dependent proteases such as Clp and FtsH are key factors in bacterial adaptation to environmental stress (Lithgow et al., 2004). It seems plausible that these bifunctional chaperones/proteases may also modulate the activity of S. aureus virulence factors.

### EXAMPLE 8

### S. aureus Mutants with Insertions in Regulatory Genes

The genome of S. aureus encodes five two-component regulatory systems and numerous transcription factors that are known to affect the production of virulence factors of S. aureus, albeit that important strain variations and differences in regulation are known to occur (Novick, 2003). Of these, three two-component regulatory systems were hit by bursa aurealis(saeRS, srrAB, agrAC), whereas insertions in arlRS were not identified (Fournier et al., 2001). The two-component regulator encoded by the yycGF genes is known to be essential for S. aureus growth (Dubrac et al., 2004). Only insertions in two of the sensory systems, saeRS and srrAB (Giraudo et al., 1999; Pragman et al., 2004), caused defects in the worm killing assay. Previously, an agrA mutant was demonstrated to display reduced virulence in the nematode killing assay (Sifri et al., 2003). Interestingly, in the current experiments, agrA, agrB, and agrC mutants did not display a consistent and significant reduction in virulence and were therefore not included in the list of virulence genes. Bursa aurealis insertions in eleven sarA homologs were isolated (Cheung et al., 2002). Surprisingly, insertion in SarA did not cause a significant defect in virulence and only insertions in the transcriptional regulator sarH2 diminished the ability of S. aureus Newman variants to kill worms. Mutations in msrR and tcaAB affect S. aureus susceptibility toward methicillin and teicoplanin and are thought to affect cell wall metabolism (Rossi et al., 2003; Brandenberger et al., 2000). Inactivation of msrR or tcaAB also affected the virulence of S. aureus during C. elegans infection.

### EXAMPLE 9

### S. aureus Mutants with Insertions in Genes Encoding Extracellular Factors

In S. aureus, synthesis and secretion of extracellular factors follows a quorum-controlled program, whereby surface adhesion genes are expressed prior to secreted hemolysins and host-tissue degradative or invasive enzymes (Novick, 2003). Only two extracellular factors, secreted nuclease and α-hemolysin, were required for the killing of C. elegans during S. aureus Newman infection. Further, attenuation of virulence was observed only with infections of L4 larvae but not with infections of adult worms, suggesting that the screening assay conditions may be too stringent to identify secreted virulence factors that play redundant or at least partially overlapping functions during pathogenesis. Furthermore, the requirement for surface proteins in this model system was directly addressed by transducing and testing mutants with bursa aurealis insertions in 10 genes encoding surface proteins. None of the insertions reduced the C. elegans virulence properties of strain Newman, not even insertions in sortase (srtA), a gene required for the cell surface display of about twenty different adhesins (Mazmanian et al., 2000; Mazmanian et al., 2002). In S. epidermidis, biofilm production has been shown to involve synthesis and secretion of PIA, a β-1,6-linked N-acetylglucosamine polysaccharide, Several mechanisms contribute to biofilm formation, which requires the icaADBC genes for PIA biosynthesis, the atlE, ssp1 and clfA genes encoding adhesive surface proteins as well as genes specifying other co-factors (dlta, aap, bap) (Gotz 2002). Although all of these genes have been hit by bursa aurealis, the isolated mutants infected and killed worms in a manner that was indistinguishable from wild-type staphylococci. Thus, sortase-anchored surface adhesins and the PIA biofilm of S. aureus are dispensable for staphylococcal replication in the C. elegans digestive track (Table 2). In contrast, insertions in genes involved in capsular polysaccharide biosynthesis, a known virulence factor in animals (Albus et al., 1991; Lee et al., 1987; Sau et al., 1997), caused reduced killing of adult worms and L4 larvae (Table 2).

### EXAMPLE 10

### Murine Infection Experiments Identify Novel S. aureus Virulence Genes

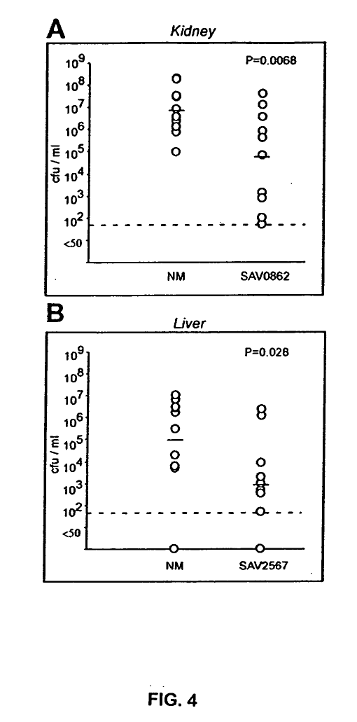

The screen identified 31 hypothetical genes that are required for the pathogenesis of S. aureus infection in C. elegans. To gauge whether these genes represent virulence factors that are involved in general disease strategies, S. aureus strains were inoculated intra-venously into mice, which were euthanized four days after infection (Albus et al., 1991). The kidney, spleen and liver were removed, inspected for abscess formation and then homogenized and plated on agar medium to quantify staphylococcal replication in host tissues. Of the eleven strains that were analyzed in this manner (Table 2), nine mutant strains caused organ abscesses in the liver and kidney in a manner that was indistinguishable from that of S. aureus wild-type strain Newman. Two strains, however, displayed significant defects in the ability to cause disease (FIG. 4). When compared to S. aureus Newman, strain ΦNΞ12478 with a bursa aurealis insertion in SAV0862, a hypothetical gene encoded by a lysogenic phage of S. aureus Newman, displayed a two log reduction in the ability to cause kidney abscesses (FIG. 4). Orf SAV2567 encodes a putative acyltransferase. Bursa aurealis insertion in SAV2567 caused a two log reduction in the ability of the mutant ΦNΞ02863 to establish liver abscesses in infected mice (FIG. 4). It should be noted that the observed defects in virulence for both strains were restricted to specific host organ tissues. ΦNΞ12478 did not show statistically significant defects in liver abscess formation, while the ΦNΞ02863 mutant caused kidney abscesses in a manner that was indistinguishable from that of wild-type strain Newman.

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE 3

| Mutant | Gene | gene | gene | gene | hit | | |
|---|---|---|---|---|---|---|---|
| Number | Number | name | begin | end | begin | Strand | Product |
| NMTN-11965 | | coa | 93 | 2165 | 651 | | |
| NMTN-13558 | | fnbB | 504 | 3394 | 1972 | | |
| NMTN-08937 | | arsC | 2383 | 2778 | 2731 | | |
| NMTN-13414 | | | 1 | 6779 | 3936 | | |
| NMTN-12542 | | splE | 4330 | 5046 | 4586 | | |
| NMTN-09697 | SAV0006 | gyrA | 7005 | 9674 | 9648 | + | DNA gyrase subunit A |
| NMTN-01964 | SAV0007 | | 9762 | 10592 | 10415 | − | conserved hypothetical protein |
| NMTN-12782d | SAV0007 | | 9762 | 10592 | 10579 | | |
| NMTN-02761 | SAV0008 | hutH | 10879 | 12414 | 11114 | + | histidine ammonia-lyase |
| NMTN-04440 | SAV0008 | hutH | 10879 | 12414 | 11596 | | |
| NMTN-09321 | SAV0010 | | 14730 | 15425 | 14879 | + | hypothetical protein |
| NMTN-06169 | SAV0010 | | 14730 | 15425 | 14919 | | |
| NMTN-02476 | SAV0012 | | 16094 | 17083 | 16919 | + | hypothetical protein |
| NMTN-12827 | SAV0012 | | 16094 | 17083 | 17025 | | |
| NMTN-13102 | SAV0013 | | 17319 | 18314 | 17463 | + | conserved hypothetical protein |
| NMTN-04509 | SAV0013 | | 17319 | 18314 | 17781 | | |
| NMTN-11727 | SAV0013 | | 17319 | 18314 | 18035 | | |
| NMTN-12713 | SAV0017 | purA | 22449 | 23732 | 22464 | + | adenylosuccinate synthase |
| NMTN-10703 | SAV0017 | purA | 22449 | 23732 | 22594 | | |
| NMTN-03581 | SAV0017 | purA | 22449 | 23732 | 22732 | | |
| NMTN-02689 | SAV0017 | purA | 22449 | 23732 | 22853 | | |
| NMTN-10572 | SAV0017 | purA | 22449 | 23732 | 22944 | | |
| NMTN-10831 | SAV0017 | purA | 22449 | 23732 | 23251 | | |
| NMTN-11456 | SAV0017 | purA | 22449 | 23732 | 23314 | | |
| NMTN-11756 | SAV0017 | purA | 22449 | 23732 | 23448 | | |
| NMTN-10932 | SAV0017 | purA | 22449 | 23732 | 23483 | | |
| NMTN-08533 | SAV0017 | purA | 22449 | 23732 | 23540 | | |
| NMTN-04972 | SAV0017 | purA | 22449 | 23732 | 23644 | | |
| NMTN-01530 | SAV0017 | purA | 22449 | 23732 | 23664 | | |
| NMTN-11611 | SAV0022 | | 29980 | 30780 | 30503 | + | conserved hypothetical protein |
| NMTN-14567 | SAV0022 | | 29980 | 30780 | 30771 | | |
| NMTN-11670 | SAV0023 | | 31007 | 33259 | 31023 | + | hypothetical protein |
| NMTN-04297 | SAV0023 | | 31007 | 33259 | 31217 | | |
| NMTN-12828 | SAV0023 | | 31007 | 33259 | 31943 | | |
| NMTN-13505 | SAV0023 | | 31007 | 33259 | 31956 | | |
| NMTN-12936 | SAV0023 | | 31007 | 33259 | 32400 | | |
| NMTN-12965 | SAV0023 | | 31007 | 33259 | 32400 | | |
| NMTN-12983d | SAV0023 | | 31007 | 33259 | 32400 | | |
| NMTN-13705 | SAV0023 | | 31007 | 33259 | 32464 | | |
| NMTN-09370 | SAV0023 | | 31007 | 33259 | 32510 | | |
| NMTN-02455 | SAV0023 | | 31007 | 33259 | 32685 | | |
| NMTN-03276 | SAV0023 | | 31007 | 33259 | 32691 | | |
| NMTN-11544 | SAV0023 | | 31007 | 33259 | 32765 | | |
| NMTN-03443 | SAV0023 | | 31007 | 33259 | 32823 | | |
| NMTN-03444 | SAV0023 | | 31007 | 33259 | 32823 | | |
| NMTN-07519 | SAV0023 | | 31007 | 33259 | 32907 | | |
| NMTN-06470 | SAV0024 | | 33697 | 34176 | 33793 | + | conserved hypothetical protein orfX |
| NMTN-00158 | SAV0024 | | 33697 | 34176 | 33928 | | |
| NMTN-00007 | SAV0031 | pre | 38433 | 39695 | 39416 | − | plasmid recombination enzyme |
| NMTN-00008 | SAV0031 | pre | 38433 | 39695 | 39416 | | |
| NMTN-06084 | SAV0071 | kdpD | 74144 | 76777 | 75938 | − | hypothetical protein |
| NMTN-12162 | SAV0082 | | 87657 | 89390 | 87668 | | |
| NMTN-02921 | SAV0082 | | 87657 | 89390 | 87959 | + | hypothetical protein |
| NMTN-09041 | SAV0082 | | 87657 | 89390 | 87959 | | |
| NMTN-03246 | SAV0082 | | 87657 | 89390 | 87961 | | |
| NMTN-06236 | SAV0082 | | 87657 | 89390 | 87964 | | |
| NMTN-13445 | SAV0082 | | 87657 | 89390 | 88428 | | |
| NMTN-01632 | SAV0082 | | 87657 | 89390 | 88458 | | |
| NMTN-02903 | SAV0082 | | 87657 | 89390 | 88505 | | |
| NMTN-04544 | SAV0082 | | 87657 | 89390 | 88508 | | |
| NMTN-09132 | SAV0082 | | 87657 | 89390 | 89116 | | |
| NMTN-02710 | SAV0084 | | 90334 | 91089 | 90441 | − | conserved hypothetical protein |
| NMTN-02569 | SAV0084 | | 90334 | 91089 | 91071 | | |
| NMTN-03605 | SAV0084 | | 90334 | 91089 | 91076 | | |
| NMTN-03606 | SAV0084 | | 90334 | 91089 | 91076 | | |
| NMTN-11469 | SAV0085 | | 91089 | 91349 | 91255 | − | conserved hypothetical protein |
| NMTN-11477 | SAV0085 | | 91089 | 91349 | 91255 | | |
| NMTN-07569 | SAV0086 | | 91486 | 92550 | 92005 | + | conserved hypothetical protein |
| NMTN-02897 | SAV0087 | | 92581 | 93915 | 92750 | + | conserved hypothetical protein |
| NMTN-04982 | SAV0087 | | 92581 | 93915 | 92836 | | |
| NMTN-11277 | SAV0087 | | 92581 | 93915 | 92951 | | |
| NMTN-04395 | SAV0087 | | 92581 | 93915 | 92972 | | |
| NMTN-13061d | SAV0087 | | 92581 | 93915 | 93034 | | |
| NMTN-06501 | SAV0087 | | 92581 | 93915 | 93679 | | |
| NMTN-11128 | SAV0088 | | 93934 | 95127 | 94213 | + | hypothetical protein |
| NMTN-01747 | SAV0088 | | 93934 | 95127 | 94509 | | |
| NMTN-04903 | SAV0088 | | 93934 | 95127 | 94655 | | |
| NMTN-11384 | SAV0088 | | 93934 | 95127 | 94730 | | |
| NMTN-06573 | SAV0088 | | 93934 | 95127 | 94897 | | |
| NMTN-06559 | SAV0088 | | 93934 | 95127 | 95048 | | |
| NMTN-12760 | SAV0088 | | 93934 | 95127 | 95060 | | |
| NMTN-12760 | SAV0088 | | 93934 | 95127 | 95060 | | |
| NMTN-00681 | SAV0088 | | 93934 | 95127 | 95068 | | |
| NMTN-06429 | SAV0089 | | 95794 | 96780 | 96641 | − | conserved hypothetical protein |
| NMTN-04416 | SAV0089 | | 95794 | 96780 | 96778 | | |
| NMTN-12169 | SAV0092 | | 97836 | 98447 | 97923 | + | hypothetical protein |
| NMTN-14422 | SAV0092 | | 97836 | 98447 | 98060 | | |
| NMTN-01701 | SAV0092 | | 97836 | 98447 | 98097 | | |
| NMTN-11771 | SAV0092 | | 97836 | 98447 | 98121 | | |
| NMTN-10640 | SAV0093 | | 98714 | 101866 | 98928 | + | hypothetical protein |
| NMTN-11846 | SAV0093 | | 98714 | 101866 | 98928 | | |
| NMTN-14664 | SAV0093 | | 98714 | 101866 | 99313 | | |
| NMTN-04522 | SAV0093 | | 98714 | 101866 | 99602 | | |
| NMTN-09017 | SAV0093 | | 98714 | 101866 | 99684 | | |
| NMTN-12303 | SAV0093 | | 98714 | 101866 | 99702 | | |
| NMTN-09022 | SAV0093 | | 98714 | 101866 | 99714 | | |
| NMTN-06265 | SAV0093 | | 98714 | 101866 | 99858 | | |
| NMTN-10867 | SAV0093 | | 98714 | 101866 | 100121 | | |
| NMTN-10580 | SAV0093 | | 98714 | 101866 | 100198 | | |
| NMTN-12233 | SAV0093 | | 98714 | 101866 | 100352 | | |
| NMTN-04969 | SAV0093 | | 98714 | 101866 | 100947 | | |
| NMTN-06215 | SAV0093 | | 98714 | 101866 | 101306 | | |
| NMTN-14503 | SAV0093 | | 98714 | 101866 | 101463 | | |
| NMTN-08742 | SAV0094 | | 101923 | 102405 | 102066 | + | hypothetical protein |
| NMTN-01578 | SAV0094 | | 101923 | 102405 | 102223 | | |
| NMTN-00113 | SAV0095 | plc | 102611 | 103597 | 103281 | + | 1-phosphatidylinositol |
| | | | | | | | phosphodiesterase precursor |
| NMTN-12336 | SAV0095 | plc | 102611 | 103597 | 103298 | | |
| NMTN-03522 | SAV0095 | plc | 102611 | 103597 | 103391 | | |
| NMTN-06798 | SAV0095 | plc | 102611 | 103597 | 103393 | | |
| NMTN-08856 | SAV0095 | plc | 102611 | 103597 | 103440 | | |
| NMTN-14570 | SAV0095 | plc | 102611 | 103597 | 103464 | | |
| NMTN-07542 | SAV0095 | plc | 102611 | 103597 | 103526 | | |
| NMTN-04331 | SAV0096 | | 103818 | 104588 | 103896 | + | hypothetical protein |
| NMTN-00584 | SAV0096 | | 103818 | 104588 | 104393 | | |
| NMTN-03737 | SAV0099 | | 106310 | 107080 | 106915 | + | hypothetical protein |
| NMTN-04701 | SAV0100 | | 107147 | 107914 | 107274 | + | hypothetical protein |
| NMTN-04709 | SAV0100 | | 107147 | 107914 | 107274 | | |
| NMTN-03975 | SAV0100 | | 107147 | 107914 | 107719 | | |
| NMTN-01752 | SAV0101 | | 108011 | 110248 | 108212 | + | hypothetical protein |
| NMTN-10945 | SAV0101 | | 108011 | 110248 | 108313 | | |
| NMTN-01586 | SAV0101 | | 108011 | 110248 | 108501 | | |
| NMTN-05318 | SAV0101 | | 108011 | 110248 | 109852 | | |
| NMTN-10988 | SAV0101 | | 108011 | 110248 | 109869 | | |
| NMTN-04135 | SAV0101 | | 108011 | 110248 | 110153 | | |
| NMTN-12459 | SAV0102 | | 110399 | 111577 | 110731 | + | hypothetical protein |
| NMTN-13495 | SAV0103 | | 111579 | 112967 | 111624 | + | hypothetical protein |
| NMTN-11065 | SAV0103 | | 111579 | 112967 | 111669 | | |
| NMTN-12838 | SAV0103 | | 111579 | 112967 | 112212 | | |
| NMTN-10450 | SAV0103 | | 111579 | 112967 | 112341 | | |
| NMTN-04675 | SAV0103 | | 111579 | 112967 | 112499 | | |
| NMTN-14391 | SAV0103 | | 111579 | 112967 | 112503 | | |
| NMTN-06613 | SAV0103 | | 111579 | 112967 | 112821 | | |
| NMTN-07033 | SAV0103 | | 111579 | 112967 | 112824 | | |
| NMTN-08903 | SAV0104 | | 113451 | 115118 | 113486 | − | conserved hypothetical protein |
| NMTN-08655 | SAV0104 | | 113451 | 115118 | 113665 | | |
| NMTN-03889 | SAV0104 | | 113451 | 115118 | 113829 | | |
| NMTN-12346 | SAV0104 | | 113451 | 115118 | 113829 | | |
| NMTN-12128 | SAV0104 | | 113451 | 115118 | 113923 | | |
| NMTN-07403 | SAV0104 | | 113451 | 115118 | 114275 | | |
| NMTN-06368 | SAV0104 | | 113451 | 115118 | 114866 | | |
| NMTN-09435 | SAV0105 | | 115210 | 115380 | 115227 | − | hypothetical protein |
| NMTN-06817 | SAV0106 | | 115437 | 117212 | 115622 | − | 67 kDa Myosin-crossreactive |
| | | | | | | | streptococcal antigen homologue |
| NMTN-11705 | SAV0106 | | 115437 | 117212 | 115700 | | |
| NMTN-01885 | SAV0106 | | 115437 | 117212 | 115810 | | |
| NMTN-07690 | SAV0106 | | 115437 | 117212 | 115810 | | |
| NMTN-07060 | SAV0106 | | 115437 | 117212 | 115815 | | |
| NMTN-05474 | SAV0107 | | 117386 | 118258 | 117389 | − | conserved hypothetical protein |
| NMTN-02908 | SAV0107 | | 117386 | 118258 | 117463 | | |
| NMTN-13514 | SAV0107 | | 117386 | 118258 | 117702 | | |
| NMTN-12584 | SAV0107 | | 117386 | 118258 | 117704 | | |
| NMTN-12353 | SAV0107 | | 117386 | 118258 | 117817 | | |
| NMTN-09396 | SAV0107 | | 117386 | 118258 | 117819 | | |
| NMTN-12785 | SAV0107 | | 117386 | 118258 | 117849 | | |
| NMTN-05622 | SAV0107 | | 117386 | 118258 | 117951 | | |
| NMTN-06307 | SAV0107 | | 117386 | 118258 | 118050 | | |
| NMTN-09023 | SAV0107 | | 117386 | 118258 | 118171 | | |
| NMTN-05033 | SAV0107 | | 117386 | 118258 | 118212 | | |
| NMTN-04993 | SAV0108 | | 118440 | 119768 | 118692 | + | hypothetical protein |
| NMTN-03596 | SAV0108 | | 118440 | 119768 | 118698 | | |
| NMTN-03867 | SAV0108 | | 118440 | 119768 | 118698 | | |
| NMTN-06920 | SAV0108 | | 118440 | 119768 | 118765 | | |
| NMTN-05484 | SAV0108 | | 118440 | 119768 | 119188 | | |
| NMTN-04045 | SAV0108 | | 118440 | 119768 | 119536 | | |
| NMTN-06065 | SAV0108 | | 118440 | 119768 | 119750 | | |
| NMTN-02760 | SAV0109 | | 119958 | 120431 | 120067 | + | hypothetical protein |
| NMTN-12136 | SAV0109 | | 119958 | 120431 | 120140 | | |
| NMTN-06243 | SAV0109 | | 119958 | 120431 | 120265 | | |
| NMTN-13863 | SAV0109 | | 119958 | 120431 | 120355 | | |
| NMTN-02284 | SAV0110 | lctP | 120693 | 122285 | 121225 | + | L-lactate permease homologue |
| NMTN-08722 | SAV0110 | lctP | 120693 | 122285 | 121394 | | |
| NMTN-04338 | SAV0110 | lctP | 120693 | 122285 | 121540 | | |
| NMTN-08768 | SAV0110 | lctP | 120693 | 122285 | 121913 | | |
| NMTN-00590 | SAV0110 | lctP | 120693 | 122285 | 121989 | | |
| NMTN-03637 | SAV0111 | spa | 122614 | 123966 | 122814 | − | Immunoglobulin G binding protein A |
| | | | | | | | precursor |
| NMTN-14676 | SAV0112 | sarH1 | 124387 | 125139 | 124714 | − | staphylococcal accessory regulator A |
| | | | | | | | homologue |
| NMTN-11086 | SAV0112 | sarH1 | 1124387 | 125139 | 124962 | | |
| NMTN-05486 | SAV0113 | sirC | 125508 | 126425 | 125535 | − | lipoprotein |
| NMTN-13610 | SAV0113 | sirC | 125508 | 126425 | 125538 | | |
| NMTN-13621 | SAV0113 | sirC | 125508 | 126425 | 125538 | | |
| NMTN-11499d | SAV0113 | sirC | 125508 | 126425 | 125776 | | |
| NMTN-12265 | SAV0113 | sirC | 125508 | 126425 | 125810 | | |
| NMTN-00251 | SAV0113 | sirC | 125508 | 126425 | 125846 | | |
| NMTN-14599 | SAV0113 | sirC | 125508 | 126425 | 125965 | | |
| NMTN-05329 | SAV0113 | sirC | 125508 | 126425 | 126164 | | |
| NMTN-09386 | SAV0113 | sirC | 125508 | 126425 | 126226 | | |
| NMTN-07861 | SAV0113 | sirC | 125508 | 126425 | 126398 | | |
| NMTN-11583 | SAV0113 | sirC | 125508 | 126425 | 126424 | | |
| NMTN-12605 | SAV0113 | sirB | 126503 | 127498 | 126533 | − | lipoprotein |
| NMTN-12605 | SAV0114 | sirB | 126503 | 127498 | 126533 | | |
| NMTN-15246 | SAV0114 | sirB | 126503 | 127498 | 127024 | | |
| NMTN-04101 | SAV0115 | sirA | 127514 | 128506 | 128217 | − | lipoprotein |
| NMTN-13438 | SAV0115 | sirA | 127514 | 128506 | 128305 | | |
| NMTN-11162 | SAV0116 | | 128737 | 129717 | 128912 | + | hypothetical protein |
| NMTN-06514 | SAV0116 | | 128737 | 129717 | 129038 | | |
| NMTN-05598 | SAV0116 | | 128737 | 129717 | 129192 | | |
| NMTN-11476 | SAV0116 | | 128737 | 129717 | 129651 | | |
| NMTN-12059 | SAV0117 | | 129714 | 130724 | 129734 | + | hypothetical protein |
| NMTN-12593 | SAV0117 | | 129714 | 130724 | 129740 | | |
| NMTN-09964 | SAV0117 | | 129714 | 130724 | 129962 | | |
| NMTN-09972 | SAV0117 | | 129714 | 130724 | 129962 | | |
| NMTN-10523 | SAV0117 | | 129714 | 130724 | 130074 | | |
| NMTN-05406 | SAV0117 | | 129714 | 130724 | 130317 | | |
| NMTN-11504 | SAV0117 | | 129714 | 130724 | 130373 | | |
| NMTN-03646 | SAV0117 | | 129714 | 130724 | 130420 | | |
| NMTN-07204 | SAV0118 | | 130745 | 132499 | 130783 | + | conserved hypothetical protein |
| NMTN-12707 | SAV0118 | | 130745 | 132499 | 130910 | | |
| NMTN-00750 | SAV0118 | | 130745 | 132499 | 130913 | | |
| NMTN-09730 | SAV0118 | | 130745 | 132499 | 131033 | | |
| NMTN-07518 | SAV0118 | | 130745 | 132499 | 131514 | | |
| NMTN-07479 | SAV0118 | | 130745 | 132499 | 131541 | | |
| NMTN-11959 | SAV0119 | | 132492 | 133748 | 132593 | + | hypothetical protein |
| NMTN-04431 | SAV0119 | | 132492 | 133748 | 132741 | | |
| NMTN-09553 | SAV0119 | | 132492 | 133748 | 133099 | | |
| NMTN-10959 | SAV0119 | | 132492 | 133748 | 133193 | | |
| NMTN-08282 | SAV0119 | | 132492 | 133748 | 133245 | | |
| NMTN-04870 | SAV0119 | | 132492 | 133748 | 133358 | | |
| NMTN-11587 | SAV0119 | | 132492 | 133748 | 133687 | | |
| NMTN-07729 | SAV0120 | | 133738 | 135474 | 133855 | + | hypothetical protein |
| NMTN-10719 | SAV0120 | | 133738 | 135474 | 134308 | | |
| NMTN-13771 | SAV0120 | | 133738 | 135474 | 134359 | | |
| NMTN-14774 | SAV0120 | | 133738 | 135474 | 134368 | | |
| NMTN-14561 | SAV0120 | | 133738 | 135474 | 134989 | | |
| NMTN-04528 | SAV0120 | | 133738 | 135474 | 135139 | | |
| NMTN-14678 | SAV0120 | | 133738 | 135474 | 135215 | | |
| NMTN-09400 | SAV0120 | | 133738 | 135474 | 135408 | | |
| NMTN-09224 | SAV0121 | | 135455 | 137233 | 135630 | + | hypothetical protein |
| NMTN-03981 | SAV0121 | | 135455 | 137233 | 135773 | | |
| NMTN-00568 | SAV0121 | | 135455 | 137233 | 136229 | | |
| NMTN-01773 | SAV0121 | | 135455 | 137233 | 136229 | | |
| NMTN-14720 | SAV0121 | | 135455 | 137233 | 137230 | | |
| NMTN-14720 | SAV0122 | | 137205 | 137984 | 137230 | + | hypothetical protein |
| NMTN-06601 | SAV0122 | | 137205 | 137984 | 137635 | | |
| NMTN-13737 | SAV0122 | | 137205 | 137984 | 137783 | | |
| NMTN-13752 | SAV0122 | | 137205 | 137984 | 137784 | | |
| NMTN-12966 | SAV0123 | | 137984 | 139186 | 138084 | + | hypothetical protein |
| NMTN-00693 | SAV0123 | | 137984 | 139186 | 139183 | | |
| NMTN-02682 | SAV0124 | | 139190 | 139954 | 139310 | + | hypothetical protein |
| NMTN-09557 | SAV0124 | | 139190 | 139954 | 139350 | | |
| NMTN-10882 | SAV0124 | | 139190 | 139954 | 139935 | | |
| NMTN-14430 | SAV0125 | | 140150 | 140776 | 140144 | + | hypothetical protein |
| NMTN-09159 | SAV0125 | | 140150 | 140776 | 140210 | | |
| NMTN-00114 | SAV0125 | | 140150 | 140776 | 140213 | | |
| NMTN-13916 | SAV0125 | | 140150 | 140776 | 140231 | | |
| NMTN-05201 | SAV0125 | | 140150 | 140776 | 140502 | | |
| NMTN-13518 | SAV0125 | | 140150 | 140776 | 140522 | | |
| NMTN-02811 | SAV0126 | butA | 140988 | 141764 | 141446 | + | acetoin(diacetyl)reductase |
| NMTN-13074 | SAV0126 | butA | 140988 | 141764 | 141721 | | |
| NMTN-08640 | SAV0128 | | 142109 | 143080 | 142441 | + | hypothetical protein |
| NMTN-06141 | SAV0128 | | 142109 | 143080 | 142604 | | |
| NMTN-09302 | SAV0128 | | 142109 | 143080 | 142756 | | |
| NMTN-00797 | SAV0128 | | 142109 | 143080 | 142763 | | |
| NMTN-07499 | SAV0129 | | 143043 | 143735 | 143401 | + | hypothetical protein |
| NMTN-02789 | SAV0129 | | 143043 | 143735 | 143686 | | |
| NMTN-07925 | SAV0129 | | 143043 | 143735 | 143702 | | |
| NMTN-09466 | SAV0130 | | 143945 | 145111 | 143961 | + | hypothetical protein |
| NMTN-11103d | SAV0130 | | 143945 | 145111 | 144713 | | |
| NMTN-13876 | SAV0130 | | 143945 | 145111 | 144935 | | |
| NMTN-01699 | SAV0130 | | 143945 | 145111 | 144938 | | |
| NMTN-09350 | SAV0131 | | 145092 | 146330 | 145300 | + | hypothetical protein |
| NMTN-12498 | SAV0131 | | 145092 | 146330 | 145506 | | |
| NMTN-08064 | SAV0131 | | 145092 | 146330 | 145706 | | |
| NMTN-08065 | SAV0131 | | 145092 | 146330 | 145708 | | |
| NMTN-12562 | SAV0131 | | 145092 | 146330 | 145832 | | |
| NMTN-00235 | SAV0131 | | 145092 | 146330 | 146074 | | |
| NMTN-00302 | SAV0131 | | 145092 | 146330 | 146074 | | |
| NMTN-06768t | SAV0131 | | 145092 | 146330 | 146120 | | |
| NMTN-09206 | SAV0132 | | 146320 | 147750 | 146719 | + | hypothetical protein |
| NMTN-14283 | SAV0132 | | 146320 | 147750 | 146785 | | |
| NMTN-00540 | SAV0132 | | 146320 | 147750 | 146886 | | |
| NMTN-05398 | SAV0132 | | 146320 | 147750 | 146903 | | |
| NMTN-02649 | SAV0132 | | 146320 | 147750 | 147010 | | |
| NMTN-09251 | SAV0132 | | 146320 | 147750 | 147028 | | |
| NMTN-01648 | SAV0132 | | 146320 | 147750 | 147316 | | |
| NMTN-02728 | SAV0133 | sodM | 148018 | 148617 | 148072 | + | superoxide dismutase |
| NMTN-10952 | SAV0134 | sasD* | 148984 | 149709 | 149223 | + | hypothetical protein |
| NMTN-01938 | SAV0134 | sasD | 148984 | 149709 | 149226 | | |
| NMTN-03734 | SAV0134 | sasD | 148984 | 149709 | 149291 | | |
| NMTN-08770 | SAV0134 | sasD | 148984 | 149709 | 149344 | | |
| NMTN-00707 | SAV0134 | sasD | 148984 | 149709 | 149552 | | |
| NMTN-00635 | SAV0134 | sasD | 148984 | 149709 | 149633 | | |
| NMTN-00256 | SAV0135 | | 149900 | 150640 | 150553 | − | hypothetical protein |
| NMTN-01871 | SAV0135 | | 149900 | 150640 | 150577 | | |
| NMTN-04115 | SAV0136 | pnp | 150891 | 151598 | 151454 | + | purine nucleoside phosphorylase |
| NMTN-06226 | SAV0137 | | 151605 | 152957 | 151776 | + | hypothetical protein |
| NMTN-04163 | SAV0137 | | 151605 | 152957 | 151961 | | |
| NMTN-11633 | SAV0137 | | 151605 | 152957 | 151961 | | |
| NMTN-01998 | SAV0137 | | 151605 | 152957 | 152170 | | |
| NMTN-02864 | SAV0137 | | 151605 | 152957 | 152402 | | |
| NMTN-02866 | SAV0137 | | 151605 | 152957 | 152402 | | |
| NMTN-02867 | SAV0137 | | 151605 | 152957 | 152402 | | |
| NMTN-03656 | SAV0137 | | 151605 | 152957 | 152402 | | |
| NMTN-12889 | SAV0137 | | 151605 | 152957 | 152402 | | |
| NMTN-15245 | SAV0137 | | 151605 | 152957 | 152916 | | |
| NMTN-13850 | SAV0138 | dra | 153038 | 153700 | 153705 | + | deoxyribose-phosphate aldolase |
| NMTN-04768 | SAV0139 | drm | 153728 | 154906 | 153945 | + | phosphopentomutase |
| NMTN-13448 | SAV0139 | drm | 153728 | 154906 | 154379 | | |
| NMTN-03891 | SAV0139 | drm | 153728 | 154906 | 154842 | | |
| NMTN-04628 | SAV0140 | | 155035 | 155850 | 155067 | − | hypothetical protein |
| NMTN-04858 | SAV0140 | | 155035 | 155850 | 155067 | | |
| NMTN-04556 | SAV0140 | | 155035 | 155850 | 155205 | | |
| NMTN-09372 | SAV0140 | | 155035 | 155850 | 155650 | | |
| NMTN-09378 | SAV0140 | | 155035 | 155850 | 155650 | | |
| NMTN-07182 | SAV0140 | | 155035 | 155850 | 155838 | | |
| NMTN-07883 | SAV0141 | | 155847 | 156647 | 155902 | − | hypothetical protein |
| NMTN-04840 | SAV0141 | | 155847 | 156647 | 156038 | | |
| NMTN-07545 | SAV0141 | | 155847 | 156647 | 156263 | | |
| NMTN-04813 | SAV0142 | | 156649 | 157422 | 156686 | − | hypothetical protein |
| NMTN-12568 | SAV0142 | | 156649 | 157422 | 156694 | | |
| NMTN-03501 | SAV0142 | | 156649 | 157422 | 157003 | | |
| NMTN-05093 | SAV0143 | | 157617 | 158573 | 158258 | − | hypothetical protein |
| NMTN-00754 | SAV0143 | | 157617 | 158573 | 158302 | | |
| NMTN-04512 | SAV0143 | | 157617 | 158573 | 158476 | | |
| NMTN-00009 | SAV0144 | | 158802 | 160346 | 158804 | + | hypothetical protein |
| NMTN-00019 | SAV0144 | | 158802 | 160346 | 158815 | | |
| NMTN-00822 | SAV0144 | | 158802 | 160346 | 159340 | | |
| NMTN-05609 | SAV0144 | | 158802 | 160346 | 159454 | | |
| NMTN-06305 | SAV0144 | | 158802 | 160346 | 159555 | | |
| NMTN-09385 | SAV0144 | | 158802 | 160346 | 159668 | | |
| NMTN-03513 | SAV0144 | | 158802 | 160346 | 159829 | | |
| NMTN-09233 | SAV0144 | | 158802 | 160346 | 160080 | | |
| NMTN-13020 | SAV0144 | | 158802 | 160346 | 160080 | | |
| NMTN-11527 | SAV0145 | | 160397 | 161932 | 160409 | + | hypothetical protein |
| NMTN-06375 | SAV0145 | | 160397 | 161932 | 161066 | | |
| NMTN-12594 | SAV0145 | | 160397 | 161932 | 161068 | | |
| NMTN-11012 | SAV0145 | | 160397 | 161932 | 161654 | | |
| NMTN-07766 | SAV0145 | | 160397 | 161932 | 161812 | | |
| NMTN-11933 | SAV0145 | | 160397 | 161932 | 161880 | | |
| NMTN-09395 | SAV0147 | | 162862 | 164037 | 163828 | + | hypothetical protein |
| NMTN-07291 | SAV0147 | | 162862 | 164037 | 163849 | | |
| NMTN-06399 | SAV0147 | | 162862 | 164037 | 164045 | | |
| NMTN-08262 | SAV0148 | adhE | 164422 | 167031 | 164641 | + | alcohol-acetaldehyde dehydrogenase |
| NMTN-13252 | SAV0148 | adhE | 164422 | 167031 | 164679 | | |
| NMTN-01814 | SAV0148 | adhE | 164422 | 167031 | 164948 | | |
| NMTN-00652 | SAV0148 | adhE | 164422 | 167031 | 165301 | | |
| NMTN-01590 | SAV0148 | adhE | 164422 | 167031 | 165946 | | |
| NMTN-12618 | SAV0148 | adhE | 164422 | 167031 | 165946 | | |
| NMTN-11427 | SAV0148 | adhE | 164422 | 167031 | 166431 | | |
| NMTN-08701 | SAV0148 | adhE | 164422 | 167031 | 166793 | | |
| NMTN-07517 | SAV0148 | adhE | 164422 | 167031 | 166799 | | |
| NMTN-11236 | SAV0148 | adhE | 164422 | 167031 | 166838 | | |
| NMTN-05613 | SAV0148 | adhE | 164422 | 167031 | 167019 | | |
| NMTN-10032 | SAV0149 | capA | 167374 | 168042 | 167388 | + | capsular polysaccharide synthesis |
| | | | | | | | enzyme Cap5A |
| NMTN-13572 | SAV0149 | capA | 167374 | 168042 | 167939 | | |
| NMTN-09402 | SAV0150 | capB | 168058 | 168744 | 168118 | + | capsular polysaccharide synthesis |
| | | | | | | | enzyme Cap5B |
| NMTN-05254 | SAV0150 | capB | 168058 | 168744 | 168676 | | |
| NMTN-05268 | SAV0150 | capB | 168058 | 168744 | 168676 | | |
| NMTN-05138 | SAV0151 | capC | 168747 | 169511 | 168769 | + | capsular polysaccharide synthesis |
| | | | | | | | enzyme Cap8C |
| NMTN-07374 | SAV0151 | capC | 168747 | 169511 | 169192 | | |
| NMTN-12543 | SAV0151 | capC | 168747 | 169511 | 169405 | | |
| NMTN-10890 | SAV0152 | capD | 169531 | 171354 | 169823 | + | capsular polysaccharide synthesis |
| | | | | | | | enzyme Cap5D |
| NMTN-14629 | SAV0152 | capD | 169531 | 171354 | 169943 | | |
| NMTN-14637d | SAV0152 | capD | 169531 | 171354 | 169943 | | |
| NMTN-10711 | SAV0152 | capD | 169531 | 171354 | 170355 | | |
| NMTN-02051 | SAV0152 | capD | 169531 | 171354 | 170544 | | |
| NMTN-11025 | SAV0152 | capD | 169531 | 171354 | 170598 | | |
| NMTN-11033 | SAV0152 | capD | 169531 | 171354 | 170620 | | |
| NMTN-14665 | SAV0152 | capD | 169531 | 171354 | 171244 | | |
| NMTN-12392 | SAV0153 | capE | 171344 | 172372 | 171866 | + | capsular polysaccharide synthesis |
| | | | | | | | enzyme Cap8E |
| NMTN-11309 | SAV0153 | capE | 171344 | 172372 | 172272 | | |
| NMTN-06712 | SAV0154 | capF | 172385 | 173494 | 172732 | + | capsular polysaccharide synthesis |
| | | | | | | | enzyme Cap5F |
| NMTN-05448 | SAV0154 | capF | 172385 | 173494 | 173142 | | |
| NMTN-06887 | SAV0154 | capF | 172385 | 173494 | 173432 | | |
| NMTN-05573 | SAV0155 | capG | 173498 | 174622 | 173597 | + | capsular polysaccharide synthesis |
| | | | | | | | enzyme Cap5G |
| NMTN-00004 | SAV0155 | capG | 173498 | 174622 | 173701 | | |
| NMTN-12183 | SAV0155 | capG | 173498 | 174622 | 174443 | | |
| NMTN-11624 | SAV0156 | capH | 174625 | 175251 | 174749 | + | capsular polysaccharide synthesis |
| | | | | | | | enzyme O-acetyl transferase Cap5H |
| NMTN-12659 | SAV0156 | capH | 174625 | 175251 | 174957 | | |
| NMTN-04851 | SAV0157 | capI | 175256 | 176365 | 175688 | + | capsular polysaccharide synthesis |
| | | | | | | | enzyme Cap5I |
| NMTN-06161 | SAV0157 | capI | 175256 | 176365 | 176129 | | |
| NMTN-10865 | SAV0158 | capJ | 176379 | 177545 | 176815 | + | capsular polysaccharide synthesis |
| | | | | | | | enzyme Cap5J |
| NMTN-06124 | SAV0158 | capJ | 176379 | 177545 | 176940 | | |
| NMTN-12264 | SAV0158 | capJ | 176379 | 177545 | 177077 | | |
| NMTN-03492 | SAV0158 | capJ | 176379 | 177545 | 177159 | | |
| NMTN-04856 | SAV0159 | capK | 177538 | 178743 | 177688 | + | capsular polysaccharide synthesis |
| | | | | | | | enzyme Cap5K |
| NMTN-10993 | SAV0159 | capK | 177538 | 178743 | 177706 | | |
| NMTN-04144 | SAV0159 | capK | 177538 | 178743 | 178073 | | |
| NMTN-07711 | SAV0159 | capK | 177538 | 178743 | 178105 | | |
| NMTN-06095 | SAV0159 | capK | 177538 | 178743 | 178316 | | |
| NMTN-13537 | SAV0159 | capK | 177538 | 178743 | 178418 | | |
| NMTN-01929 | SAV0159 | capK | 177538 | 178743 | 178490 | | |
| NMTN-12805 | SAV0159 | capK | 177538 | 178743 | 178492 | | |
| NMTN-12976 | SAV0159 | capK | 177538 | 178743 | 178492 | | |
| NMTN-08155 | SAV0159 | capK | 177538 | 178743 | 178493 | | |
| NMTN-06218 | SAV0159 | capK | 177538 | 178743 | 178495 | | |
| NMTN-13318 | SAV0159 | capK | 177538 | 178743 | 178600 | | |
| NMTN-06426 | SAV0160 | capL | 178744 | 179949 | 179152 | + | capsular polysaccharide synthesis |
| | | | | | | | enzyme Cap5L |
| NMTN-07750 | SAV0160 | capL | 178744 | 179949 | 179466 | | |
| NMTN-00618 | SAV0160 | capL | 178744 | 179949 | 179744 | | |
| NMTN-10879 | SAV0161 | capM | 179960 | 180517 | 180039 | + | capsular polysaccharide synthesis |
| | | | | | | | enzyme Cap5M |
| NMTN-01957 | SAV0161 | capM | 179960 | 180517 | 180123 | | |
| NMTN-01866 | SAV0162 | capN | 180517 | 181404 | 180581 | + | capsular polysaccharide synthesis |
| | | | | | | | enzyme Cap5N |
| NMTN-04136 | SAV0162 | capN | 180517 | 181404 | 180722 | | |
| NMTN-06083 | SAV0163 | capO | 181458 | 182720 | 181875 | + | capsular polysaccharide synthesis |
| | | | | | | | enzyme Cap8O |
| NMTN-05283 | SAV0163 | capO | 181458 | 182720 | 182050 | | |
| NMTN-12917 | SAV0163 | capO | 181458 | 182720 | 182254 | | |
| NMTN-11669 | SAV0163 | capO | 181458 | 182720 | 182382 | | |
| NMTN-04646 | SAV0163 | capO | 181458 | 182720 | 182489 | | |
| NMTN-08890 | SAV0163 | capO | 181458 | 182720 | 182505 | | |
| NMTN-04700 | SAV0164 | capP | 182767 | 183942 | 182926 | + | capsular polysaccharide synthesis |
| | | | | | | | enzyme Cap5P |
| NMTN-05644 | SAV0164 | capP | 182767 | 183942 | 183501 | | |
| NMTN-04706 | SAV0164 | capP | 182767 | 183942 | 183668 | | |
| NMTN-09847 | SAV0164 | capP | 182767 | 183942 | 183792 | | |
| NMTN-09227 | SAV0165 | isdI | 184007 | 184333 | 184034 | − | conserved hypothetical protein |
| NMTN-11092 | SAV0165 | isdI | 184007 | 184333 | 184116 | | |
| NMTN-11891 | SAV0165 | isdI | 184007 | 184333 | 184170 | | |
| NMTN-12932 | SAV0166 | | 184340 | 184723 | 184477 | − | conserved hypothetical protein |
| NMTN-04350 | SAV0166 | isdG** | 184340 | 184723 | 184481 | | |
| NMTN-07301 | SAV0167 | aldA | 185150 | 186637 | 185141 | | |
| NMTN-15192 | SAV0167 | aldA | 185150 | 186637 | 185864 | + | aldehyde dehydrogenase homologue |
| NMTN-02715 | SAV0167 | aldA | 185150 | 186637 | 185925 | | |
| NMTN-06813 | SAV0167 | aldA | 185150 | 186637 | 186032 | | |
| NMTN-03273 | SAV0168 | | 187284 | 188243 | 187488 | + | hypothetical protein |
| NMTN-08279 | SAV0168 | | 187284 | 188243 | 187999 | | |
| NMTN-11701 | SAV0168 | | 187284 | 188243 | 188031 | | |
| NMTN-13649 | SAV0168 | | 187284 | 188243 | 188031 | | |
| NMTN-05237 | SAV0171 | | 188920 | 189432 | 189318 | + | hypothetical protein |
| NMTN-07506 | SAV0172 | | 189774 | 190514 | 189822 | + | hypothetical protein |
| NMTN-02635 | SAV0172 | | 189774 | 190514 | 189925 | | |
| NMTN-10948 | SAV0172 | | 189774 | 190514 | 189955 | | |
| NMTN-07554 | SAV0173 | | 190528 | 191499 | 190627 | + | hypothetical protein |
| NMTN-11245 | SAV0173 | | 190528 | 191499 | 190670 | | |
| NMTN-07471 | SAV0173 | | 190528 | 191499 | 190976 | | |
| NMTN-03639 | SAV0173 | | 190528 | 191499 | 191031 | | |
| NMTN-07902 | SAV0173 | | 190528 | 191499 | 191290 | | |
| NMTN-06072 | SAV0174 | | 191496 | 192257 | 191601 | + | hypothetical protein |
| NMTN-05563 | SAV0174 | | 191496 | 192257 | 191653 | | |
| NMTN-00861 | SAV0174 | | 191496 | 192257 | 192197 | | |
| NMTN-00698 | SAV0175 | | 192270 | 193301 | 192626 | + | hypothetical protein |
| NMTN-13362 | SAV0175 | | 192270 | 193301 | 192996 | | |
| NMTN-05305 | SAV0175 | | 192270 | 193301 | 193064 | | |
| NMTN-10577 | SAV0175 | | 192270 | 193301 | 193308 | | |
| NMTN-02233 | SAV0176 | | 193527 | 193877 | 193609 | + | conserved hypothetical protein |
| NMTN-10905 | SAV0176 | | 193527 | 193877 | 193609 | | |
| NMTN-07878 | SAV0177 | fdh | 193952 | 195076 | 194048 | + | NAD-dependent formate |
| | | | | | | | dehydrogenase |
| NMTN-07526 | SAV0177 | fdh | 193952 | 195076 | 194251 | | |
| NMTN-10911 | SAV0178 | | 195462 | 196712 | 195675 | + | hypothetical protein |
| NMTN-02469 | SAV0178 | | 195462 | 196712 | 196090 | | |
| NMTN-09014 | SAV0178 | | 195462 | 196712 | 196575 | | |
| NMTN-12202 | SAV0179 | | 197159 | 204334 | 197203 | + | hypothetical protein |
| NMTN-13575 | SAV0179 | | 197159 | 204334 | 197467 | | |
| NMTN-00016 | SAV0179 | | 197159 | 204334 | 197654 | | |
| NMTN-04439 | SAV0179 | | 197159 | 204334 | 197900 | | |
| NMTN-02049 | SAV0179 | | 197159 | 204334 | 197901 | | |
| NMTN-06946 | SAV0179 | | 197159 | 204334 | 197909 | | |
| NMTN-07190 | SAV0179 | | 197159 | 204334 | 197909 | | |
| NMTN-04853 | SAV0179 | | 197159 | 204334 | 197981 | | |
| NMTN-11262 | SAV0179 | | 197159 | 204334 | 198566 | | |
| NMTN-06633 | SAV0179 | | 197159 | 204334 | 199169 | | |
| NMTN-10667 | SAV0179 | | 197159 | 204334 | 199707 | | |
| NMTN-00811 | SAV0179 | | 197159 | 204334 | 199809 | | |
| NMTN-14695 | SAV0179 | | 197159 | 204334 | 199870 | | |
| NMTN-03234 | SAV0179 | | 197159 | 204334 | 200255 | | |
| NMTN-02766 | SAV0179 | | 197159 | 204334 | 200320 | | |
| NMTN-01820 | SAV0179 | | 197159 | 204334 | 200478 | | |
| NMTN-11721 | SAV0179 | | 197159 | 204334 | 200478 | | |
| NMTN-04446 | SAV0179 | | 197159 | 204334 | 200546 | | |
| NMTN-01854 | SAV0179 | | 197159 | 204334 | 200622 | | |
| NMTN-04641 | SAV0179 | | 197159 | 204334 | 200697 | | |
| NMTN-13211 | SAV0179 | | 197159 | 204334 | 201206 | | |
| NMTN-01891 | SAV0179 | | 197159 | 204334 | 201264 | | |
| NMTN-08612 | SAV0179 | | 197159 | 204334 | 201293 | | |
| NMTN-04365 | SAV0179 | | 197159 | 204334 | 201295 | | |
| NMTN-07825 | SAV0179 | | 197159 | 204334 | 201336 | | |
| NMTN-08323 | SAV0179 | | 197159 | 204334 | 201571 | | |
| NMTN-06437 | SAV0179 | | 197159 | 204334 | 201599 | | |
| NMTN-09393 | SAV0179 | | 197159 | 204334 | 201686 | | |
| NMTN-09666 | SAV0179 | | 197159 | 204334 | 201837 | | |
| NMTN-07956 | SAV0179 | | 197159 | 204334 | 202174 | | |
| NMTN-06385 | SAV0179 | | 197159 | 204334 | 202221 | | |
| NMTN-04381 | SAV0179 | | 197159 | 204334 | 202435 | | |
| NMTN-11449 | SAV0179 | | 197159 | 204334 | 203118 | | |
| NMTN-08671 | SAV0179 | | 197159 | 204334 | 203142 | | |
| NMTN-11184 | SAV0179 | | 197159 | 204334 | 203142 | | |
| NMTN-06485 | SAV0179 | | 197159 | 204334 | 203169 | | |
| NMTN-08756 | SAV0179 | | 197159 | 204334 | 203505 | | |
| NMTN-13062 | SAV0179 | | 197159 | 204334 | 203660 | | |
| NMTN-06835 | SAV0179 | | 197159 | 204334 | 203665 | | |
| NMTN-03491 | SAV0179 | | 197159 | 204334 | 203769 | | |
| NMTN-09354 | SAV0179 | | 197159 | 204334 | 203822 | | |
| NMTN-11799 | SAV0179 | | 197159 | 204334 | 203946 | | |
| NMTN-10880 | SAV0179 | | 197159 | 204334 | 204189 | | |
| NMTN-14532 | SAV0180 | | 204347 | 204991 | 204448 | + | conserved hypothetical protein |
| NMTN-03096 | SAV0180 | | 204347 | 204991 | 204753 | | |
| NMTN-10581 | SAV0182 | | 206068 | 206832 | 206317 | − | hypothetical protein |
| NMTN-06304 | SAV0182 | | 206068 | 206832 | 206488 | | |
| NMTN-03414 | SAV0182 | | 206068 | 206832 | 206538 | | |
| NMTN-02265 | SAV0182 | | 206068 | 206832 | 206795 | | |
| NMTN-12640 | SAV0183 | argJ | 206848 | 208089 | 208055 | − | arginine biosynthesis bifunctional |
| | | | | | | | protein homologue |
| NMTN-03127 | SAV0184 | argC | 208101 | 209132 | 208251 | − | N-acetylglutamate gamma− |
| | | | | | | | semialdehyde dehydrogenase |
| NMTN-02803 | SAV0184 | argC | 208101 | 209132 | 208612 | | |
| NMTN-07307 | SAV0184 | argC | 208101 | 209132 | 208985 | | |
| NMTN-02387 | SAV0185 | | 209171 | 210355 | 209256 | − | ornithine aminotransferase |
| NMTN-00824 | SAV0185 | | 209171 | 210355 | 210144 | | |
| NMTN-05074 | SAV0185 | | 209171 | 210355 | 210235 | | |
| NMTN-12376 | SAV0186 | | 210614 | 211963 | 210942 | − | hypothetical protein |
| NMTN-13213 | SAV0186 | | 210614 | 211963 | 211268 | | |
| NMTN-10656 | SAV0186 | | 210614 | 211963 | 211715 | | |
| NMTN-08784 | SAV0187 | | 212242 | 212739 | 212621 | − | hypothetical protein |
| NMTN-09016 | SAV0188 | | 212866 | 214506 | 213464 | − | hypothetical protein |
| NMTN-02579 | SAV0188 | | 212866 | 214506 | 213929 | | |
| NMTN-07755 | SAV0188 | | 212866 | 214506 | 213929 | | |
| NMTN-03140 | SAV0188 | | 212866 | 214506 | 213931 | | |
| NMTN-06532 | SAV0188 | | 212866 | 214506 | 214099 | | |
| NMTN-12556 | SAV0188 | | 212866 | 214506 | 214148 | | |
| NMTN-06454 | SAV0189 | glcA | 214778 | 216823 | 215026 | − | PTS enzyme II |
| NMTN-03102 | SAV0189 | glcA | 214778 | 216823 | 215642 | | |
| NMTN-05296 | SAV0189 | glcA | 214778 | 216823 | 215704 | | |
| NMTN-01753 | SAV0189 | glcA | 214778 | 216823 | 216550 | | |
| NMTN-04337 | SAV0190 | | 217408 | 218463 | 217432 | + | conserved hypothetical protein |
| NMTN-00178 | SAV0190 | | 217408 | 218463 | 217985 | | |
| NMTN-12312 | SAV0190 | | 217408 | 218463 | 218168 | | |
| NMTN-11922 | SAV0192 | | 219371 | 220825 | 219473 | + | hypothetical protein |
| NMTN-00006 | SAV0192 | | 219371 | 220825 | 219577 | | |
| NMTN-01763 | SAV0192 | | 219371 | 220825 | 219623 | | |
| NMTN-04956 | SAV0192 | | 219371 | 220825 | 219682 | | |
| NMTN-00863 | SAV0192 | | 219371 | 220825 | 220038 | | |
| NMTN-04361 | SAV0192 | | 219371 | 220825 | 220347 | | |
| NMTN-04951 | SAV0192 | | 219371 | 220825 | 220347 | | |
| NMTN-03927 | SAV0192 | | 219371 | 220825 | 220650 | | |
| NMTN-13067 | SAV0193 | | 220828 | 221703 | 220906 | + | hypothetical protein |
| NMTN-02135 | SAV0193 | | 220828 | 221703 | 220967 | | |
| NMTN-08828 | SAV0193 | | 220828 | 221703 | 221067 | | |
| NMTN-02362 | SAV0193 | | 220828 | 221703 | 221456 | | |
| NMTN-02429 | SAV0195 | hsdR | 222391 | 225180 | 222479 | + | probable type I restriction enzyme |
| | | | | | | | restriction chain |
| NMTN-13443 | SAV0195 | hsdR | 222391 | 225180 | 222743 | | |
| NMTN-12699 | SAV0195 | hsdR | 222391 | 225180 | 222911 | | |
| NMTN-00710 | SAV0195 | hsdR | 222391 | 225180 | 223058 | | |
| NMTN-09492 | SAV0195 | hsdR | 222391 | 225180 | 223205 | | |
| NMTN-05126 | SAV0195 | hsdR | 222391 | 225180 | 223655 | | |
| NMTN-09118 | SAV0195 | hsdR | 222391 | 225180 | 223659 | | |
| NMTN-08992 | SAV0195 | hsdR | 222391 | 225180 | 223693 | | |
| NMTN-15247 | SAV0195 | hsdR | 222391 | 225180 | 224668 | | |
| NMTN-09050 | SAV0195 | hsdR | 222391 | 225180 | 224757 | | |
| NMTN-07632 | SAV0195 | hsdR | 222391 | 225180 | 224854 | | |
| NMTN-14427 | SAV0195 | hsdR | 222391 | 225180 | 224946 | | |
| NMTN-08861 | SAV0205 | oppF | 233309 | 234901 | 233824 | − | oligopeptide transport ATP-binding |
| | | | | | | | protein |
| NMTN-11739 | SAV0205 | oppF | 233309 | 234901 | 234790 | | |
| NMTN-10862 | SAV0206 | truncated- | 234999 | 236336 | 235224 | + | truncated hypothetical protein |
| | | oppB | | | | | |
| NMTN-12437 | SAV0206 | truncated- | 234999 | 236336 | 235540 | | |
| | | oppB | | | | | |
| NMTN-07849 | SAV0206 | truncated- | 234999 | 236336 | 235961 | | |
| | | oppB | | | | | |
| NMTN-11430 | SAV0206 | truncated- | 234999 | 236336 | 236240 | | |
| | | oppB | | | | | |
| NMTN-14610 | SAV0206 | truncated- | 234999 | 236336 | 236250 | | |
| | | oppB | | | | | |
| NMTN-04004 | SAV0207 | | 236342 | 237505 | 236667 | + | hypothetical protein |
| NMTN-05315 | SAV0207 | | 236342 | 237505 | 236994 | | |
| NMTN-05454 | SAV0207 | | 236342 | 237505 | 237185 | | |
| NMTN-06052 | SAV0207 | | 236342 | 237505 | 237299 | | |
| NMTN-11760 | SAV0207 | | 236342 | 237505 | 237381 | | |
| NMTN-01926 | SAV0208 | rlp | 237522 | 239297 | 237570 | + | RGD-containing lipoprotein |
| NMTN-07795 | SAV0208 | rlp | 237522 | 239297 | 237571 | | |
| NMTN-12525 | SAV0208 | rlp | 237522 | 239297 | 238567 | | |
| NMTN-03751 | SAV0209 | | 239335 | 241341 | 239494 | + | hypothetical protein |
| NMTN-03761 | SAV0209 | | 239335 | 241341 | 239494 | | |
| NMTN-03750 | SAV0209/ | | 239335 | 241341 | 239494 | | |
| | SAV0103 | | | | | | |
| NMTN-07928 | SAV0209 | | 239335 | 241341 | 239578 | | |
| NMTN-03566 | SAV0209 | | 239335 | 241341 | 239639 | | |
| NMTN-15333 | SAV0209 | | 239335 | 241341 | 239657 | | |
| NMTN-13268 | SAV0209 | | 239335 | 241341 | 239863 | | |
| NMTN-12316 | SAV0209 | | 239335 | 241341 | 239967 | | |
| NMTN-13896 | SAV0209 | | 239335 | 241341 | 241037 | | |
| NMTN-05324 | SAV0209 | | 239335 | 241341 | 241261 | | |
| NMTN-08994 | SAV0209 | | 239335 | 241341 | 241271 | | |
| NMTN-02891 | SAV0209 | | 239335 | 241341 | 241278 | | |
| NMTN-06988 | SAV0209 | | 239335 | 241341 | 241296 | | |
| NMTN-00603 | SAV0209 | | 239335 | 241341 | 241311 | | |
| NMTN-12945 | SAV0210 | | 241654 | 242427 | 241925 | − | hypothetical protein |
| NMTN-11515 | SAV0210 | | 241654 | 242427 | 242150 | | |
| NMTN-07427 | SAV0210 | | 241654 | 242427 | 242203 | | |
| NMTN-07298 | SAV0210 | | 241654 | 242427 | 242206 | | |
| NMTN-13516 | SAV0210 | | 241654 | 242427 | 242242 | | |
| NMTN-12256 | SAV0211 | | 242616 | 243242 | 242966 | − | NAD(P)H dehydrogenase homologue |
| NMTN-09004 | SAV0211 | | 242616 | 243242 | 242967 | | |
| NMTN-07811 | SAV0211 | | 242616 | 243242 | 243049 | | |
| NMTN-10874 | SAV0211 | | 242616 | 243242 | 243156 | | |
| NMTN-10277 | SAV0211 | | 242616 | 243242 | 243244 | | |
| NMTN-11857 | SAV0212 | | 243451 | 244029 | 243505 | + | hypothetical protein |
| NMTN-04162 | SAV0212 | | 243451 | 244029 | 243523 | | |
| NMTN-06068 | SAV0212 | | 243451 | 244029 | 243581 | | |
| NMTN-13778 | SAV0212 | | 243451 | 244029 | 243651 | | |
| NMTN-10402 | SAV0212 | | 243451 | 244029 | 243801 | | |
| NMTN-10403 | SAV0212 | | 243451 | 244029 | 243801 | | |
| NMTN-10405 | SAV0212 | | 243451 | 244029 | 243801 | | |
| NMTN-10410 | SAV0212 | | 243451 | 244029 | 243801 | | |
| NMTN-10411 | SAV0212 | | 243451 | 244029 | 243801 | | |
| NMTN-10419 | SAV0212 | | 243451 | 244029 | 243801 | | |
| NMTN-10420 | SAV0212 | | 243451 | 244029 | 243801 | | |
| NMTN-10423 | SAV0212 | | 243451 | 244029 | 243801 | | |
| NMTN-10428 | SAV0212 | | 243451 | 244029 | 243801 | | |
| NMTN-10430 | SAV0212 | | 243451 | 244029 | 243801 | | |
| NMTN-10460 | SAV0212 | | 243451 | 244029 | 243801 | | |
| NMTN-10462 | SAV0212 | | 243451 | 244029 | 243801 | | |
| NMTN-11738 | SAV0212 | | 243451 | 244029 | 243836 | | |
| NMTN-13376 | SAV0212 | | 243451 | 244029 | 243837 | | |
| NMTN-06383 | SAV0213 | msmX | 244413 | 245510 | 244684 | + | multiple sugar-binding transport ATP- |
| | | | | | | | binding protein |
| NMTN-08675 | SAV0213 | msmX | 244413 | 245510 | 244864 | | |
| NMTN-06618 | SAV0213 | msmX | 244413 | 245510 | 245076 | | |
| NMTN-12348 | SAV0213 | msmX | 244413 | 245510 | 245097 | | |
| NMTN-06972 | SAV0213 | msmX | 244413 | 245510 | 245467 | | |
| NMTN-03834 | SAV0214 | | 245523 | 246794 | 245568 | + | hypothetical protein |
| NMTN-14479 | SAV0214 | | 245523 | 246794 | 245996 | | |
| NMTN-12263 | SAV0214 | | 245523 | 246794 | 246580 | | |
| NMTN-08291 | SAV0215 | | 246797 | 248065 | 247574 | + | maltose/maltodextrin transport |
| | | | | | | | permease homologue |
| NMTN-13547 | SAV0215 | | 246797 | 248065 | 247607 | | |
| NMTN-12504 | SAV0216 | | 248067 | 248906 | 248139 | + | maltose/maltodextrin transport |
| | | | | | | | permease homologue |
| NMTN-02950 | SAV0216 | | 248067 | 248906 | 248287 | | |
| NMTN-03194 | SAV0216 | | 248067 | 248906 | 248298 | | |
| NMTN-13904 | SAV0216 | | 248067 | 248906 | 248348 | | |
| NMTN-00051 | SAV0216 | | 248067 | 248906 | 248447 | | |
| NMTN-01515 | SAV0216 | | 248067 | 248906 | 248629 | | |
| NMTN-12364 | SAV0217 | | 248977 | 250056 | 249316 | + | hypothetical protein |
| NMTN-04534 | SAV0218 | | 250081 | 251121 | 250298 | + | hypothetical protein |
| NMTN-04548 | SAV0218 | | 250081 | 251121 | 250298 | | |
| NMTN-11575 | SAV0218 | | 250081 | 251121 | 250363 | | |
| NMTN-12211 | SAV0218 | | 250081 | 251121 | 250445 | | |
| NMTN-09449 | SAV0218 | | 250081 | 251121 | 250692 | | |
| NMTN-08362 | SAV0218 | | 250081 | 251121 | 251059 | | |
| NMTN-01858 | SAV0218 | | 250081 | 251121 | 251071 | | |
| NMTN-12429 | SAV0218 | | 250081 | 251121 | 251128 | | |
| NMTN-03836 | SAV0219 | | 251176 | 252144 | 251228 | + | conserved hypothetical protein |
| NMTN-11513 | SAV0219 | | 251176 | 252144 | 251238 | | |
| NMTN-01499 | SAV0219 | | 251176 | 252144 | 251714 | | |
| NMTN-05230 | SAV0219 | | 251176 | 252144 | 252024 | | |
| NMTN-12663 | SAV0220 | | 252505 | 252999 | 252780 | − | conserved hypothetical protein |
| NMTN-11730 | SAV0220 | | 252505 | 252999 | 252847 | | |
| NMTN-04364 | SAV0220 | | 252505 | 252999 | 252977 | | |
| NMTN-08995 | SAV0222 | uhpT | 253232 | 253951 | 253056 | + | hexose phosphate transport protein |
| NMTN-10780 | SAV0222 | uhpT | 253232 | 253951 | 253416 | | |
| NMTN-07443 | SAV0222 | uhpT | 253232 | 253951 | 253594 | | |
| NMTN-05413 | SAV0222 | uhpT | 253232 | 254611 | 253784 | | |
| NMTN-00616 | SAV0222 | uhpT | 253955 | 254611 | 254128 | | |
| NMTN-01873 | SAV0222 | uhpT | 253955 | 254611 | 254186 | | |
| NMTN-01889 | SAV0222 | uhpT | 253955 | 254611 | 254186 | | |
| NMTN-00592 | SAV0222 | uhpT | 253955 | 254611 | 254527 | | |
| NMTN-00716 | SAV0222 | uhpT | 253955 | 254611 | 254527 | | |
| NMTN-00717 | SAV0222 | uhpT | 253955 | 254611 | 254527 | | |
| NMTN-09482 | SAV0223 | | 254970 | 255728 | 255509 | − | hypothetical protein |
| NMTN-12010 | SAV0223 | | 254970 | 255728 | 255633 | | |
| NMTN-00452 | SAV0223 | | 254970 | 255728 | 255719 | | |
| NMTN-02933 | SAV0224 | | 255721 | 257277 | 255729 | − | hypothetical protein |
| NMTN-09571 | SAV0224 | | 255721 | 257277 | 256205 | | |
| NMTN-07304 | SAV0224 | | 255721 | 257277 | 256284 | | |
| NMTN-09538 | SAV0224 | | 255721 | 257277 | 256840 | | |
| NMTN-07461 | SAV0224 | | 255721 | 257277 | 256923 | | |
| NMTN-11248 | SAV0224 | | 255721 | 257277 | 257167 | | |
| NMTN-12666 | SAV0225 | | 257274 | 258242 | 257626 | − | hypothetical protein |
| NMTN-09513 | SAV0225 | | 257274 | 258242 | 257720 | | |
| NMTN-02423 | SAV0225 | | 257274 | 258242 | 257728 | | |
| NMTN-00232 | SAV0225 | | 257274 | 258242 | 257889 | | |
| NMTN-13472 | SAV0226 | pflB | 258830 | 261079 | 258836 | + | formate acetyltransferase |
| NMTN-13723 | SAV0226 | pflB | 258830 | 261079 | 258899 | | |
| NMTN-14058 | SAV0226 | pflB | 258830 | 261079 | 258899 | | |
| NMTN-05431 | SAV0226 | pflB | 258830 | 261079 | 259130 | | |
| NMTN-12999 | SAV0226 | pflB | 258830 | 261079 | 259446 | | |
| NMTN-07509 | SAV0226 | pflB | 258830 | 261079 | 259600 | | |
| NMTN-03884 | SAV0226 | pflB | 258830 | 261079 | 260237 | | |
| NMTN-13055 | SAV0226 | pflB | 258830 | 261079 | 260373 | | |
| NMTN-05414 | SAV0226 | pflB | 258830 | 261079 | 260769 | | |
| NMTN-13028 | SAV0228 | | 262179 | 263942 | 262923 | + | hypothetical protein |
| NMTN-04690 | SAV0228 | | 262179 | 263942 | 263028 | | |
| NMTN-09373 | SAV0228 | | 262179 | 263942 | 263168 | | |
| NMTN-09379 | SAV0228 | | 262179 | 263942 | 263168 | | |
| NMTN-12997 | SAV0228 | | 262179 | 263942 | 263784 | | |
| NMTN-14033 | SAV0229 | | 264106 | 264450 | 264341 | − | hypothetical protein |
| NMTN-12830 | SAV0230 | coa | 264640 | 266616 | 265432 | + | staphylocoagulase precursor |
| NMTN-00492 | SAV0230 | coa | 264640 | 266616 | 265946 | | |
| NMTN-02852 | SAV0230 | coa | 264640 | 266616 | 266235 | | |
| NMTN-06144 | SAV0231 | | 267203 | 268387 | 267397 | − | acetyl-CoA acetyltransferase |
| | | | | | | | homologue |
| NMTN-11460 | SAV0231 | | 267203 | 268387 | 267881 | | |
| NMTN-04061 | SAV0231 | | 267203 | 268387 | 267919 | | |
| NMTN-03669 | SAV0231 | | 267203 | 268387 | 267937 | | |
| NMTN-08823 | SAV0231 | | 267203 | 268387 | 267950 | | |
| NMTN-02733 | SAV0231 | | 267203 | 268387 | 267961 | | |
| NMTN-07137d | SAV0231 | | 267203 | 268387 | 268048 | | |
| NMTN-13014d | SAV0232 | | 268417 | 270678 | 268525 | − | hypothetical protein |
| NMTN-14533 | SAV0232 | | 268417 | 270678 | 268708 | | |
| NMTN-12082 | SAV0232 | | 268417 | 270678 | 268980 | | |
| NMTN-13462 | SAV0232 | | 268417 | 270678 | 268980 | | |
| NMTN-09715 | SAV0232 | | 268417 | 270678 | 269108 | | |
| NMTN-12806 | SAV0232 | | 268417 | 270678 | 269405 | | |
| NMTN-09034 | SAV0232 | | 268417 | 270678 | 269645 | | |
| NMTN-05362 | SAV0232 | | 268417 | 270678 | 269980 | | |
| NMTN-10972 | SAV0232 | | 268417 | 270678 | 270098 | | |
| NMTN-08134 | SAV0232 | | 268417 | 270678 | 270185 | | |
| NMTN-01985 | SAV0232 | | 268417 | 270678 | 270230 | | |
| NMTN-01842 | SAV0232 | | 268417 | 270678 | 270423 | | |
| NMTN-00054 | SAV0232 | | 268417 | 270678 | 270498 | | |
| NMTN-12864 | SAV0233 | | 270864 | 272075 | 271000 | − | hypothetical protein |
| NMTN-06234 | SAV0233 | | 270864 | 272075 | 271191 | | |
| NMTN-09469 | SAV0233 | | 270864 | 272075 | 271281 | | |
| NMTN-14512 | SAV0233 | | 270864 | 272075 | 271281 | | |
| NMTN-03997 | SAV0233 | | 270864 | 272075 | 271523 | | |
| NMTN-09518 | SAV0233 | | 270864 | 272075 | 271761 | | |
| NMTN-11991 | SAV0233 | | 270864 | 272075 | 272079 | | |
| NMTN-11657 | SAV0234 | | 272187 | 273692 | 272269 | − | hypothetical protein |
| NMTN-06845 | SAV0234 | | 272187 | 273692 | 272369 | | |
| NMTN-04666 | SAV0234 | | 272187 | 273692 | 272971 | | |
| NMTN-09223 | SAV0235 | | 273718 | 275280 | 273709 | − | conserved hypothetical protein |
| NMTN-00796 | SAV0235 | | 273718 | 275280 | 274053 | | |
| NMTN-03764 | SAV0235 | | 273718 | 275280 | 274595 | | |
| NMTN-08577 | SAV0235 | | 273718 | 275280 | 274931 | | |
| NMTN-11834d | SAV0235 | | 273718 | 275280 | 274961 | | |
| NMTN-00015 | SAV0236 | | 275736 | 276878 | 275953 | + | hypothetical protein |
| NMTN-13802 | SAV0236 | | 275736 | 276878 | 276226 | | |
| NMTN-12972 | SAV0236 | | 275736 | 276878 | 276591 | | |
| NMTN-03838 | SAV0236 | | 275736 | 276878 | 276708 | | |
| NMTN-11407 | SAV0237 | | 277192 | 278667 | 277229 | − | hypothetical protein |
| NMTN-07202 | SAV0237 | | 277192 | 278667 | 277667 | | |
| NMTN-09207 | SAV0237 | | 277192 | 278667 | 277759 | | |
| NMTN-09520 | SAV0237 | | 277192 | 278667 | 277874 | | |
| NMTN-11454 | SAV0237 | | 277192 | 278667 | 277895 | | |
| NMTN-03665 | SAV0237 | | 277192 | 278667 | 277901 | | |
| NMTN-06982 | SAV0237 | | 277192 | 278667 | 278200 | | |
| NMTN-01810 | SAV0237 | | 277192 | 278667 | 278344 | | |
| NMTN-11473 | SAV0238 | | 278865 | 279221 | 279187 | − | conserved hypothetical protein |
| NMTN-06088 | SAV0240 | | 279577 | 280722 | 279828 | − | hypothetical protein |
| NMTN-11180 | SAV0240 | | 279577 | 280722 | 279909 | | |
| NMTN-05886 | SAV0240 | | 279577 | 280722 | 279959 | | |
| NMTN-09163 | SAV0240 | | 279577 | 280722 | 280130 | | |
| NMTN-04861 | SAV0240 | | 279577 | 280722 | 280138 | | |
| NMTN-14418 | SAV0240 | | 279577 | 280722 | 280303 | | |
| NMTN-11105 | SAV0240 | | 279577 | 280722 | 280386 | | |
| NMTN-02831 | SAV0241 | lctE | 281295 | 282248 | 281357 | + | L-lactate dehydrogenase |
| NMTN-11741 | SAV0241 | lctE | 281295 | 282248 | 281727 | | |
| NMTN-06726t | SAV0241 | lctE | 281295 | 282248 | 281990 | | |
| NMTN-12870 | SAV0242 | | 282568 | 284097 | 283082 | − | PTS enzyme |
| NMTN-11718 | SAV0242 | | 282568 | 284097 | 283359 | | |
| NMTN-11720 | SAV0242 | | 282568 | 284097 | 283359 | | |
| NMTN-08010 | SAV0242 | | 282568 | 284097 | 283588 | | |
| NMTN-10824 | SAV0242 | | 282568 | 284097 | 283773 | | |
| NMTN-02365 | SAV0243 | | 284459 | 285394 | 285173 | + | hypothetical protein |
| NMTN-12294 | SAV0244 | | 285731 | 287827 | 286723 | + | hypothetical protein |
| NMTN-10705 | SAV0244 | | 285731 | 287827 | 286897 | | |
| NMTN-09375 | SAV0244 | | 285731 | 287827 | 287477 | | |
| NMTN-09381 | SAV0244 | | 285731 | 287827 | 287477 | | |
| NMTN-14293 | SAV0244 | | 285731 | 287827 | 287780 | | |
| NMTN-03950 | SAV0245 | | 287812 | 288279 | 288125 | + | hypothetical protein |
| NMTN-13901 | SAV0245 | | 287812 | 288279 | 288148 | | |
| NMTN-06720 | SAV0245 | | 287812 | 288279 | 288272 | | |
| NMTN-05635 | SAV0246 | | 288302 | 288580 | 288320 | + | hypothetical protein |
| NMTN-13103 | SAV0247 | gatC | 288807 | 290066 | 289951 | + | probable PTS galactitol-specific enzyme |
| | | | | | | | IIC component |
| NMTN-05298 | SAV0248 | | 290084 | 291139 | 290258 | + | sorbitol dehydrogenase |
| NMTN-02456 | SAV0248 | | 290084 | 291139 | 290347 | | |
| NMTN-10639 | SAV0248 | | 290084 | 291139 | 290350 | | |
| NMTN-10934 | SAV0248 | | 290084 | 291139 | 290350 | | |
| NMTN-08061 | SAV0248 | | 290084 | 291139 | 290462 | | |
| NMTN-05235 | SAV0250 | | 291311 | 292354 | 291399 | + | sorbitol dehydrogenase homologue |
| NMTN-12703 | SAV0250 | | 291311 | 292354 | 291463 | | |
| NMTN-02563 | SAV0250 | | 291311 | 292354 | 291506 | | |
| NMTN-00243 | SAV0250 | | 291311 | 292354 | 291643 | | |
| NMTN-12539 | SAV0250 | | 291311 | 292354 | 291884 | | |
| NMTN-10860 | SAV0250 | | 291311 | 292354 | 291885 | | |
| NMTN-02654 | SAV0250 | | 291311 | 292354 | 291890 | | |
| NMTN-04049 | SAV0250 | | 291311 | 292354 | 291890 | | |
| NMTN-09085 | SAV0250 | | 291311 | 292354 | 291906 | | |
| NMTN-12737 | SAV0250 | | 291311 | 292354 | 292007 | | |
| NMTN-01506 | SAV0250 | | 291311 | 292354 | 292222 | | |
| NMTN-15241 | SAV0250 | | 291311 | 292354 | 292257 | | |
| NMTN-01934 | SAV0251 | | 292882 | 293598 | 293284 | + | hypothetical protein |
| NMTN-07739 | SAV0252 | | 293591 | 294616 | 294169 | + | hypothetical protein |
| NMTN-07972 | SAV0252 | | 293591 | 294616 | 294192 | | |
| NMTN-05236 | SAV0252 | | 293591 | 294616 | 294477 | | |
| NMTN-06414 | SAV0252 | | 293591 | 294616 | 294533 | | |
| NMTN-06545 | SAV0253 | | 294638 | 296332 | 294857 | + | hypothetical protein |
| NMTN-04632 | SAV0253 | | 294638 | 296332 | 295374 | | |
| NMTN-03224 | SAV0258 | | 301682 | 303403 | 301700 | + | hypothetical protein |
| NMTN-07206 | SAV0258 | | 301682 | 303403 | 302075 | | |
| NMTN-04126 | SAV0258 | | 301682 | 303403 | 302528 | | |
| NMTN-08462 | SAV0258 | | 301682 | 303403 | 302661 | | |
| NMTN-06310 | SAV0258 | | 301682 | 303403 | 302928 | | |
| NMTN-06910 | SAV0258 | | 301682 | 303403 | 303100 | | |
| NMTN-00118 | SAV0259 | scdA | 303547 | 304221 | 303944 | + | cell division and morphogenesis-related |
| | | | | | | | protein |
| NMTN-02542 | SAV0259 | scdA | 303547 | 304221 | 304003 | | |
| NMTN-02543 | SAV0259 | scdA | 303547 | 304221 | 304003 | | |
| NMTN-04188 | SAV0259 | scdA | 303547 | 304221 | 304094 | | |
| NMTN-12116 | SAV0259 | scdA | 303547 | 304221 | 304094 | | |
| NMTN-11393 | SAV0259 | scdA | 303547 | 304221 | 304201 | | |
| NMTN-02846 | SAV0260 | lytS | 304466 | 306220 | 304602 | + | two-component sensor histidine kinase |
| NMTN-08540 | SAV0260 | lytS | 304466 | 306220 | 304602 | | |
| NMTN-03944 | SAV0260 | lytS | 304466 | 306220 | 304810 | | |
| NMTN-13256 | SAV0260 | lytS | 304466 | 306220 | 304851 | | |
| NMTN-07386 | SAV0260 | lytS | 304466 | 306220 | 305276 | | |
| NMTN-00645 | SAV0260 | lytS | 304466 | 306220 | 305741 | | |
| NMTN-05796 | SAV0261 | lytR | 306223 | 306963 | 306277 | + | two-component response regulator |
| NMTN-04539 | SAV0261 | lytR | 306223 | 306963 | 306830 | | |
| NMTN-06542 | SAV0261 | lytR | 306223 | 306963 | 306957 | | |
| NMTN-08827 | SAV0262 | lrgA | 307082 | 307519 | 307235 | + | holin-like protein |
| NMTN-00684 | SAV0262 | lrgA | 307082 | 307519 | 307409 | | |
| NMTN-12275 | SAV0264 | | 308321 | 309025 | 308741 | − | hypothetical protein |
| NMTN-05502 | SAV0264 | | 308321 | 309025 | 309014 | | |
| NMTN-13853 | SAV0265 | | 309174 | 309965 | 309184 | + | hypothetical protein |
| NMTN-11126 | SAV0265 | | 309174 | 309965 | 309757 | | |
| NMTN-10715 | SAV0265 | | 309174 | 309965 | 309777 | | |
| NMTN-10852 | SAV0266 | bglA | 309981 | 311417 | 310166 | + | 6-phospho-beta-glucosidase |
| NMTN-12650 | SAV0266 | bglA | 309981 | 311417 | 311312 | | |
| NMTN-11711 | SAV0267 | | 311904 | 312665 | 311910 | − | conserved hypothetical protein |
| NMTN-08331 | SAV0267 | | 311904 | 312665 | 311922 | | |
| NMTN-12288 | SAV0267 | | 311904 | 312665 | 312403 | | |
| NMTN-08149 | SAV0267 | | 311904 | 312665 | 312581 | | |
| NMTN-09554 | SAV0267 | | 311904 | 312665 | 312646 | | |
| NMTN-09840 | SAV0268 | rbsK | 312916 | 313830 | 313312 | − | probable ribokinase |
| NMTN-07989 | SAV0269 | rbsD | 313858 | 314262 | 314040 | − | ribose permease |
| NMTN-05444 | SAV0270 | | 314277 | 315158 | 314338 | − | hypothetical protein |
| NMTN-08976 | SAV0270 | | 314277 | 315158 | 314506 | | |
| NMTN-09036 | SAV0270 | | 314277 | 315158 | 314509 | | |
| NMTN-07943 | SAV0270 | | 314277 | 315158 | 315162 | | |
| NMTN-03680 | SAV0271 | | 315390 | 316388 | 315393 | − | hypothetical protein |
| NMTN-07021 | SAV0271 | | 315390 | 316388 | 315541 | | |
| NMTN-12824 | SAV0271 | | 315390 | 316388 | 316020 | | |
| NMTN-05434 | SAV0271 | | 315390 | 316388 | 316031 | | |
| NMTN-03097 | SAV0271 | | 315390 | 316388 | 316321 | | |
| NMTN-11540 | SAV0271 | | 315390 | 316388 | 316393 | | |
| NMTN-07234 | SAV0272 | | 316527 | 316727 | 316697 | + | NADH dehydrogenase subunit |
| NMTN-12766 | SAV0273 | | 316765 | 317157 | 316815 | + | hypothetical protein |
| NMTN-04884 | SAV0274 | | 317285 | 318661 | 317347 | − | hypothetical protein |
| NMTN-11282 | SAV0274 | | 317285 | 318661 | 317434 | | |
| NMTN-07031 | SAV0274 | | 317285 | 318661 | 317496 | | |
| NMTN-10840 | SAV0274 | | 317285 | 318661 | 318057 | | |
| NMTN-00275 | SAV0274 | | 317285 | 318661 | 318145 | | |
| NMTN-09562 | SAV0274 | | 317285 | 318661 | 318150 | | |
| NMTN-14451 | SAV0274 | | 317285 | 318661 | 318241 | | |
| NMTN-06195 | SAV0274 | | 317285 | 318661 | 318376 | | |
| NMTN-01870 | SAV0275 | | 318895 | 319887 | 319279 | + | hypothetical protein |
| NMTN-05529 | SAV0275 | | 318895 | 319887 | 319592 | | |
| NMTN-06423 | SAV0275 | | 318895 | 319887 | 319715 | | |
| NMTN-02987 | SAV0276 | lytM | 320213 | 321163 | 320262 | + | peptidoglycan hydrolase |
| NMTN-13071 | SAV0276 | lytM | 320213 | 321163 | 320364 | | |
| NMTN-07010 | SAV0276 | lytM | 320213 | 321163 | 320801 | | |
| NMTN-10851 | SAV0277 | | 321215 | 321874 | 321224 | − | conserved hypothetical protein |
| NMTN-04752 | SAV0277 | | 321215 | 321874 | 321460 | | |
| NMTN-12831 | SAV0277 | | 321215 | 321874 | 321495 | | |
| NMTN-04665 | SAV0277 | | 321215 | 321874 | 321549 | | |
| NMTN-10915 | SAV0277 | | 321215 | 321874 | 321778 | | |
| NMTN-05460 | SAV0278 | | 321888 | 322808 | 321908 | − | hypothetical protein |
| NMTN-12519 | SAV0278 | | 321888 | 322808 | 321934 | | |
| NMTN-07287 | SAV0278 | | 321888 | 322808 | 321937 | | |
| NMTN-11434 | SAV0278 | | 321888 | 322808 | 322015 | | |
| NMTN-05175 | SAV0278 | | 321888 | 322808 | 322120 | | |
| NMTN-13402d | SAV0278 | | 321888 | 322808 | 322400 | | |
| NMTN-00493 | SAV0278 | | 321888 | 322808 | 322444 | | |
| NMTN-05550 | SAV0278 | | 321888 | 322808 | 322720 | | |
| NMTN-01681 | SAV0279 | | 322805 | 323971 | 322898 | − | hypothetical protein |
| NMTN-00381 | SAV0279 | | 322805 | 323971 | 323117 | | |
| NMTN-10664 | SAV0279 | | 322805 | 323971 | 323298 | | |
| NMTN-00629 | SAV0279 | | 322805 | 323971 | 323604 | | |
| NMTN-13243 | SAV0279 | | 322805 | 323971 | 323612 | | |
| NMTN-07198 | SAV0279 | | 322805 | 323971 | 323625 | | |
| NMTN-01970 | SAV0279 | | 322805 | 323971 | 323824 | | |
| NMTN-04368 | SAV0279 | | 322805 | 323971 | 323906 | | |
| NMTN-11750 | SAV0280 | | 324039 | 325562 | 324319 | − | hypothetical protein |
| NMTN-06456 | SAV0280 | | 324039 | 325562 | 324411 | | |
| NMTN-04994 | SAV0280 | | 324039 | 325562 | 324451 | | |
| NMTN-03794 | SAV0280 | | 324039 | 325562 | 324809 | | |
| NMTN-13900 | SAV0280 | | 324039 | 325562 | 325010 | | |
| NMTN-10570 | SAV0280 | | 324039 | 325562 | 325061 | | |
| NMTN-14462 | SAV0281 | | 325893 | 326786 | 326019 | | |
| NMTN-14809 | SAV0281 | | 325893 | 326786 | 326019 | − | hypothetical protein |
| NMTN-02936 | SAV0281 | | 325893 | 326786 | 326184 | | |
| NMTN-07490 | SAV0281 | | 325893 | 326786 | 326184 | | |
| NMTN-14682 | SAV0283 | | 327410 | 330439 | 327923 | + | hypothetical protein |
| NMTN-09349 | SAV0283 | | 327410 | 330439 | 328600 | | |
| NMTN-15360 | SAV0283 | | 327410 | 330439 | 330247 | | |
| NMTN-06301 | SAV0283 | | 327410 | 330439 | 330304 | | |
| NMTN-03742 | SAV0284 | | 330439 | 330897 | 330560 | + | hypothetical protein |
| NMTN-13152 | SAV0285 | | 330869 | 331111 | 330912 | + | conserved hypothetical protein |
| NMTN-11241 | SAV0285 | | 330869 | 331111 | 330968 | | |
| NMTN-02411 | SAV0286 | | 331124 | 332458 | 331322 | + | conserved hypothetical protein |
| NMTN-11556 | SAV0286 | | 331124 | 332458 | 331322 | | |
| NMTN-15284 | SAV0286 | | 331124 | 332458 | 331340 | | |
| NMTN-05513 | SAV0286 | | 331124 | 332458 | 331452 | | |
| NMTN-00689 | SAV0286 | | 331124 | 332458 | 331890 | | |
| NMTN-00165 | SAV0286 | | 331124 | 332458 | 331950 | | |
| NMTN-00394 | SAV0286 | | 331124 | 332458 | 331950 | | |
| NMTN-00511 | SAV0286 | | 331124 | 332458 | 331950 | | |
| NMTN-07525 | SAV0286 | | 331124 | 332458 | 332163 | | |
| NMTN-10892 | SAV0286 | | 331124 | 332458 | 332458 | | |
| NMTN-06491 | SAV0287 | | 332480 | 333679 | 332547 | + | conserved hypothetical protein |
| NMTN-07893 | SAV0287 | | 332480 | 333679 | 332566 | | |
| NMTN-03683 | SAV0287 | | 332480 | 333679 | 332895 | | |
| NMTN-04464 | SAV0287 | | 332480 | 333679 | 333271 | | |
| NMTN-11214 | SAV0288 | | 333672 | 336920 | 333773 | + | conserved hypothetical protein |
| NMTN-02755 | SAV0288 | | 333672 | 336920 | 334140 | | |
| NMTN-02191 | SAV0288 | | 333672 | 336920 | 334334 | | |
| NMTN-02832 | SAV0288 | | 333672 | 336920 | 334641 | | |
| NMTN-10633 | SAV0288 | | 333672 | 336920 | 335132 | | |
| NMTN-11255 | SAV0288 | | 333672 | 336920 | 335135 | | |
| NMTN-13592 | SAV0288 | | 333672 | 336920 | 335364 | | |
| NMTN-04847 | SAV0288 | | 333672 | 336920 | 336087 | | |
| NMTN-05618 | SAV0288 | | 333672 | 336920 | 336152 | | |
| NMTN-13038 | SAV0288 | | 333672 | 336920 | 336315 | | |
| NMTN-13043 | SAV0288 | | 333672 | 336920 | 336738 | | |
| NMTN-08872 | SAV0288 | | 333672 | 336920 | 336799 | | |
| NMTN-09488 | SAV0288 | | 333672 | 336920 | 336856 | | |
| NMTN-07912 | SAV0290 | | 337358 | 337672 | 337359 | | |
| NMTN-08567 | SAV0290 | | 337358 | 337672 | 337612 | + | hypothetical protein |
| NMTN-08340 | SAV0293 | | 338673 | 340517 | 338691 | + | conserved hypothetical protein |
| NMTN-11068 | SAV0293 | | 338673 | 340517 | 338775 | | |
| NMTN-07570 | SAV0293 | | 338673 | 340517 | 338953 | | |
| NMTN-13293 | SAV0293 | | 338673 | 340517 | 338958 | | |
| NMTN-03493 | SAV0293 | | 338673 | 340517 | 338970 | | |
| NMTN-09277 | SAV0293 | | 338673 | 340517 | 339676 | | |
| NMTN-11663 | SAV0293 | | 338673 | 340517 | 339724 | | |
| NMTN-03911 | SAV0293 | | 338673 | 340517 | 340122 | | |
| NMTN-08495 | SAV0293 | | 338673 | 340517 | 340195 | | |
| NMTN-10978 | SAV0294 | | 340528 | 341019 | 341000 | + | conserved hypothetical protein |
| NMTN-13089 | SAV0295 | | 341222 | 341905 | 341220 | + | hypothetical protein |
| NMTN-04048 | SAV0295 | | 341222 | 341905 | 341247 | | |
| NMTN-13694 | SAV0295 | | 341222 | 341905 | 341248 | | |
| NMTN-06126 | SAV0295 | | 341222 | 341905 | 341598 | | |
| NMTN-06822 | SAV0295 | | 341222 | 341905 | 341712 | | |
| NMTN-12070 | SAV0295 | | 341222 | 341905 | 341846 | | |
| NMTN-00500 | SAV0295 | | 341222 | 341905 | 341857 | | |
| NMTN-07742 | SAV0296 | | 342051 | 342662 | 342145 | + | hypothetical protein |
| NMTN-13914 | SAV0296 | | 342051 | 342662 | 342152 | | |
| NMTN-09573 | SAV0296 | | 342051 | 342662 | 342193 | | |
| NMTN-05120 | SAV0296 | | 342051 | 342662 | 342495 | | |
| NMTN-04317 | SAV0297 | | 342797 | 343015 | 342915 | + | hypothetical protein |
| NMTN-05433 | SAV0298 | | 343147 | 343647 | 343317 | + | conserved hypothetical protein |
| NMTN-10048 | SAV0298 | | 343147 | 343647 | 343320 | | |
| NMTN-02932 | SAV0298 | | 343147 | 343647 | 343493 | | |
| NMTN-04791 | SAV0298 | | 343147 | 343647 | 343544 | | |
| NMTN-06103 | SAV0299 | | 343658 | 344158 | 343759 | + | conserved hypothetical protein |
| NMTN-11844 | SAV0300 | | 344169 | 344630 | 344582 | + | conserved hypothetical protein |
| NMTN-06534 | SAV0301 | | 344679 | 345179 | 344885 | + | conserved hypothetical protein |
| NMTN-06634 | SAV0302 | | 345190 | 345675 | 345538 | + | conserved hypothetical protein |
| NMTN-10684 | SAV0302 | | 345190 | 345675 | 345582 | | |
| NMTN-10920 | SAV0302 | | 345190 | 345675 | 345625 | | |
| NMTN-06648 | SAV0302 | | 345190 | 345675 | 345653 | | |
| NMTN-08543 | SAV0303 | | 346300 | 346674 | 346682 | + | hypothetical protein |
| NMTN-06396 | SAV0304 | | 346824 | 347222 | 347078 | + | hypothetical protein |
| NMTN-07501 | SAV0304 | | 346824 | 347222 | 347103 | | |
| NMTN-03864 | SAV0304 | | 346824 | 347222 | 347178 | | |
| NMTN-12220 | SAV0305 | | 347438 | 348262 | 347929 | − | hypothetical protein |
| NMTN-00605 | SAV0305 | | 347438 | 348262 | 347977 | | |
| NMTN-09719 | SAV0305 | | 347438 | 348262 | 348228 | | |
| NMTN-14709 | SAV0306 | | 348499 | 349806 | 348660 | − | hypothetical protein |
| NMTN-05155 | SAV0306 | | 348499 | 349806 | 348897 | | |
| NMTN-03413 | SAV0306 | | 348499 | 349806 | 349093 | | |
| NMTN-12867 | SAV0306 | | 348499 | 349806 | 349118 | | |
| NMTN-03089 | SAV0307 | | 350390 | 351280 | 350410 | + | hypothetical protein |
| NMTN-12439 | SAV0307 | | 350390 | 351280 | 350410 | | |
| NMTN-07388 | SAV0307 | | 350390 | 351280 | 350669 | | |
| NMTN-02028 | SAV0307 | | 350390 | 351280 | 350750 | | |
| NMTN-02029 | SAV0307 | | 350390 | 351280 | 350750 | | |
| NMTN-04783 | SAV0307 | | 350390 | 351280 | 350982 | | |
| NMTN-08120 | SAV0307 | | 350390 | 351280 | 351098 | | |
| NMTN-07352 | SAV0307 | | 350390 | 351280 | 351138 | | |
| NMTN-06476 | SAV0308 | | 351529 | 352578 | 351916 | + | conserved hypothetical protein |
| NMTN-00460 | SAV0308 | | 351529 | 352578 | 352279 | | |
| NMTN-02480 | SAV0308 | | 351529 | 352578 | 352515 | | |
| NMTN-13888 | SAV0309 | | 352591 | 353268 | 353054 | + | hypothetical protein |
| NMTN-04052 | SAV0310 | | 353476 | 354507 | 353495 | + | hypothetical protein |
| NMTN-07777 | SAV0310 | | 353476 | 354507 | 353518 | | |
| NMTN-08817 | SAV0310 | | 353476 | 354507 | 353743 | | |
| NMTN-05210 | SAV0310 | | 353476 | 354507 | 353745 | | |
| NMTN-00853 | SAV0310 | | 353476 | 354507 | 354387 | | |
| NMTN-02805 | SAV0311 | | 354850 | 355968 | 355283 | + | hypothetical protein |
| NMTN-04086 | SAV0311 | | 354850 | 355968 | 355298 | | |
| NMTN-13883 | SAV0311 | | 354850 | 355968 | 355739 | | |
| NMTN-00108 | SAV0311 | | 354850 | 355968 | 355834 | | |
| NMTN-08765 | SAV0312 | | 355943 | 356866 | 356127 | + | conserved hypothetical protein |
| NMTN-10783 | SAV0312 | | 355943 | 356866 | 356136 | | |
| NMTN-06575 | SAV0312 | | 355943 | 356866 | 356671 | | |
| NMTN-07340 | SAV0312 | | 355943 | 356866 | 356671 | | |
| NMTN-05564 | SAV0313 | | 356877 | 358097 | 357244 | + | probable pyrimidine nucleoside |
| | | | | | | | transport protein |
| NMTN-10826 | SAV0313 | | 356877 | 358097 | 357373 | | |
| NMTN-03125 | SAV0313 | | 356877 | 358097 | 357417 | | |
| NMTN-04110 | SAV0313 | | 356877 | 358097 | 357425 | | |
| NMTN-02707 | SAV0313 | | 356877 | 358097 | 357684 | | |
| NMTN-02708 | SAV0313 | | 356877 | 358097 | 357684 | | |
| NMTN-04713 | SAV0313 | | 356877 | 358097 | 357691 | | |
| NMTN-03193 | SAV0313 | | 356877 | 358097 | 357693 | | |
| NMTN-13466 | SAV0313 | | 356877 | 358097 | 357916 | | |
| NMTN-07746 | SAV0314 | | 358202 | 359734 | 358195 | − | hypothetical protein |
| NMTN-10632 | SAV0314 | | 358202 | 359734 | 358344 | | |
| NMTN-06445 | SAV0314 | | 358202 | 359734 | 358390 | | |
| NMTN-08831 | SAV0314 | | 358202 | 359734 | 358803 | | |
| NMTN-07456 | SAV0314 | | 358202 | 359734 | 358863 | | |
| NMTN-03729 | SAV0314 | | 358202 | 359734 | 358935 | | |
| NMTN-09701 | SAV0314 | | 358202 | 359734 | 359162 | | |
| NMTN-11215 | SAV0314 | | 358202 | 359734 | 359373 | | |
| NMTN-13697 | SAV0314 | | 358202 | 359734 | 359460 | | |
| NMTN-09108 | SAV0315 | nanA | 359774 | 360655 | 360323 | − | N-acetylneuraminate lyase subunit |
| NMTN-02871 | SAV0316 | | 360813 | 361673 | 360818 | + | hypothetical protein |
| NMTN-08818 | SAV0316 | | 360813 | 361673 | 360954 | | |
| NMTN-11934 | SAV0316 | | 360813 | 361673 | 361003 | | |
| NMTN-06650 | SAV0316 | | 360813 | 361673 | 361408 | | |
| NMTN-14683 | SAV0317 | | 361950 | 362750 | 362584 | − | conserved hypothetical protein |
| NMTN-08080 | SAV0317 | | 361950 | 362750 | 362691 | | |
| NMTN-13133 | SAV0318 | | 362890 | 363558 | 362949 | + | conserved hypothetical protein |
| NMTN-13880 | SAV0318 | | 362890 | 363558 | 363217 | | |
| NMTN-03752 | SAV0318 | | 362890 | 363558 | 363222 | | |
| NMTN-06525 | SAV0318 | | 362890 | 363558 | 363338 | | |
| NMTN-07516 | SAV0318 | | 362890 | 363558 | 363453 | | |
| NMTN-06115 | SAV0319 | | 363689 | 365002 | 364533 | − | conserved hypothetical protein |
| NMTN-09174 | SAV0319 | | 363689 | 365002 | 364908 | | |
| NMTN-00040 | SAV0320 | geh | 365420 | 367495 | 366732 | + | glycerol ester hydrolase |
| NMTN-08795 | SAV0321 | | 367737 | 368564 | 367969 | − | hypothetical protein |
| NMTN-15244 | SAV0322 | | 368637 | 369836 | 369085 | − | hypothetical protein |
| NMTN-12426d | SAV0322 | | 368637 | 369836 | 369838 | | |
| NMTN-07336 | SAV0323 | | 370138 | 371139 | 370447 | + | hypothetical protein |
| NMTN-10723 | SAV0323 | | 370138 | 371139 | 370480 | | |
| NMTN-07949 | SAV0323 | | 370138 | 371139 | 370660 | | |
| NMTN-12538 | SAV0323 | | 370138 | 371139 | 370748 | | |
| NMTN-11496 | SAV0324 | | 371173 | 371505 | 371231 | + | hypothetical protein |
| NMTN-05241 | SAV0325 | | 371506 | 372306 | 371698 | + | conserved hypothetical protein |
| NMTN-09128 | SAV0325 | | 371506 | 372306 | 371855 | | |
| NMTN-11482 | SAV0325 | | 371506 | 372306 | 372204 | | |
| NMTN-14522 | SAV0325 | | 371506 | 372306 | 372247 | | |
| NMTN-04742 | SAV0326 | | 372293 | 373240 | 372306 | + | hypothetical protein |
| NMTN-07407 | SAV0326 | | 372293 | 373240 | 372984 | | |
| NMTN-01707 | SAV0326 | | 372293 | 373240 | 373071 | | |
| NMTN-07189 | SAV0326 | | 372293 | 373240 | 373212 | | |
| NMTN-11764 | SAV0327 | | 373218 | 374240 | 373400 | | |
| NMTN-02961 | SAV0327 | | 373218 | 374240 | 373460 | + | hypothetical protein |
| NMTN-04339 | SAV0327 | | 373218 | 374240 | 373561 | | |
| NMTN-12083 | SAV0327 | | 373218 | 374240 | 374243 | | |
| NMTN-08706 | SAV0327 | | 373218 | 374240 | 374247 | | |
| NMTN-08707 | SAV0327 | | 373218 | 374240 | 374247 | | |
| NMTN-06073 | SAV0328 | | 374556 | 375581 | 375132 | + | hypothetical protein |
| NMTN-13813 | SAV0328 | | 374556 | 375581 | 375240 | | |
| NMTN-06254 | SAV0328 | | 374556 | 375581 | 375475 | | |
| NMTN-02086 | SAV0329 | | 375675 | 377021 | 375736 | − | hypothetical protein |
| NMTN-11976 | SAV0329 | | 375675 | 377021 | 376153 | | |
| NMTN-04777 | SAV0329 | | 375675 | 377021 | 376175 | | |
| NMTN-05096 | SAV0329 | | 375675 | 377021 | 376207 | | |
| NMTN-10946 | SAV0329 | | 375675 | 377021 | 376351 | | |
| NMTN-07702 | SAV0329 | | 375675 | 377021 | 376484 | | |
| NMTN-05517 | SAV0329 | | 375675 | 377021 | 376516 | | |
| NMTN-11793 | SAV0329 | | 375675 | 377021 | 376556 | | |
| NMTN-09291 | SAV0329 | | 375675 | 377021 | 376754 | | |
| NMTN-11892 | SAV0329 | | 375675 | 377021 | 377030 | | |
| NMTN-11892 | SAV0330 | | 377036 | 377320 | 377030 | − | conserved hypothetical protein |
| NMTN-08728 | SAV0330 | | 377036 | 377320 | 377091 | | |
| NMTN-02277 | SAV0330 | | 377036 | 377320 | 377302 | | |
| NMTN-04848 | SAV0332 | | 377770 | 379725 | 378044 | − | hypothetical protein |
| NMTN-06548 | SAV0332 | | 377770 | 379725 | 378513 | | |
| NMTN-00014 | SAV0332 | | 377770 | 379725 | 378586 | | |
| NMTN-11684 | SAV0332 | | 377770 | 379725 | 378586 | | |
| NMTN-07503 | SAV0332 | | 377770 | 379725 | 378790 | | |
| NMTN-02510 | SAV0332 | | 377770 | 379725 | 378966 | | |
| NMTN-01509 | SAV0332 | | 377770 | 379725 | 379140 | | |
| NMTN-01510 | SAV0332 | | 377770 | 379725 | 379140 | | |
| NMTN-05320 | SAV0332 | | 377770 | 379725 | 379225 | | |
| NMTN-02134 | SAV0334 | | 380463 | 381818 | 380574 | + | conserved hypothetical protein |
| NMTN-04148 | SAV0334 | | 380463 | 381818 | 380574 | | |
| NMTN-12814 | SAV0334 | | 380463 | 381818 | 380886 | | |
| NMTN-08652 | SAV0334 | | 380463 | 381818 | 381219 | | |
| NMTN-06133 | SAV0334 | | 380463 | 381818 | 381454 | | |
| NMTN-13808 | SAV0334 | | 380463 | 381818 | 381495 | | |
| NMTN-07308 | SAV0334 | | 380463 | 381818 | 381696 | | |
| NMTN-12875 | SAV0334 | | 380463 | 381818 | 381699 | | |
| NMTN-03498 | SAV0335 | | 381922 | 382380 | 382253 | + | hypothetical protein |
| NMTN-08713 | SAV0337 | glpT | 382444 | 382719 | 382623 | − | glycerol-3-phosphate transporter |
| NMTN-05632 | SAV0337 | glpT | 382738 | 383802 | 382799 | − | glycerol-3-phosphate transporter |
| NMTN-12614 | SAV0337 | glpT | 382738 | 383802 | 382944 | | |
| NMTN-02555 | SAV0337 | glpT | 382738 | 383802 | 383214 | | |
| NMTN-07783 | SAV0337 | glpT | 382738 | 383802 | 383581 | | |
| NMTN-04867 | SAV0337 | glpT | 382738 | 383802 | 383744 | | |
| NMTN-03817 | SAV0337 | glpT | 382738 | 383802 | 383771 | | |
| NMTN-13805 | SAV0338 | | 384114 | 385040 | 384245 | + | conserved hypothetical protein |
| NMTN-08388 | SAV0338 | | 384114 | 385040 | 384418 | | |
| NMTN-13032 | SAV0338 | | 384114 | 385040 | 384535 | | |
| NMTN-12267 | SAV0338 | | 384114 | 385040 | 384815 | | |
| NMTN-02460 | SAV0338 | | 384114 | 385040 | 384904 | | |
| NMTN-10394 | SAV0339 | | 385054 | 386115 | 385169 | + | conserved hypothetical protein |
| NMTN-00413 | SAV0339 | | 385054 | 386115 | 385462 | | |
| NMTN-12206 | SAV0339 | | 385054 | 386115 | 385462 | | |
| NMTN-04056 | SAV0339 | | 385054 | 386115 | 385579 | | |
| NMTN-14638 | SAV0339 | | 385054 | 386115 | 385723 | | |
| NMTN-01514 | SAV0339 | | 385054 | 386115 | 385786 | | |
| NMTN-02044 | SAV0339 | | 385054 | 386115 | 385787 | | |
| NMTN-05037 | SAV0340 | | 386129 | 386695 | 386309 | + | hypothetical protein |
| NMTN-03829 | SAV0340 | | 386129 | 386695 | 386441 | | |
| NMTN-00622 | SAV0340 | | 386129 | 386695 | 386483 | | |
| NMTN-06448 | SAV0340 | | 386129 | 386695 | 386502 | | |
| NMTN-07595 | SAV0340 | | 386129 | 386695 | 386567 | | |
| NMTN-11379 | SAV0341 | | 386754 | 387749 | 386758 | − | conserved hypothetical protein |
| NMTN-09397 | SAV0341 | | 386754 | 387749 | 386802 | | |
| NMTN-06430 | SAV0341 | | 386754 | 387749 | 386955 | | |
| NMTN-07826 | SAV0341 | | 386754 | 387749 | 386955 | | |
| NMTN-12272 | SAV0341 | | 386754 | 387749 | 386959 | | |
| NMTN-09120 | SAV0341 | | 386754 | 387749 | 386962 | | |
| NMTN-09117 | SAV0341 | | 386754 | 387749 | 387072 | | |
| NMTN-02680 | SAV0341 | | 386754 | 387749 | 387234 | | |
| NMTN-03583 | SAV0341 | | 386754 | 387749 | 387235 | | |
| NMTN-13170 | SAV0341 | | 386754 | 387749 | 387245 | | |
| NMTN-08690 | SAV0341 | | 386754 | 387749 | 387524 | | |
| NMTN-01547 | SAV0341 | | 386754 | 387749 | 387579 | | |
| NMTN-07708 | SAV0341 | | 386754 | 387749 | 387611 | | |
| NMTN-00424 | SAV0341 | | 386754 | 387749 | 387613 | | |
| NMTN-09560 | SAV0341 | | 386754 | 387749 | 387698 | | |
| NMTN-12477 | SAV0342 | | 388114 | 388659 | 388397 | + | hypothetical protein |
| NMTN-12469 | SAV0342 | | 388114 | 388659 | 388399 | | |
| NMTN-13656 | SAV0343 | | 388926 | 389780 | 389037 | + | conserved hypothetical protein |
| NMTN-12557 | SAV0343 | | 388926 | 389780 | 389319 | | |
| NMTN-11302 | SAV0344 | | 389891 | 391006 | 390390 | + | conserved hypothetical protein |
| NMTN-05333 | SAV0344 | | 389891 | 391006 | 390642 | | |
| NMTN-06246 | SAV0344 | | 389891 | 391006 | 390922 | | |
| NMTN-02522 | SAV0344 | | 389891 | 391006 | 390924 | | |
| NMTN-07975 | SAV0344 | | 389891 | 391006 | 390929 | | |
| NMTN-08222 | SAV0344 | | 389891 | 391006 | 390938 | | |
| NMTN-11665 | SAV0345 | | 390984 | 392699 | 391045 | + | conserved hypothetical protein |
| NMTN-14527 | SAV0345 | | 390984 | 392699 | 391542 | | |
| NMTN-10980 | SAV0345 | | 390984 | 392699 | 391804 | | |
| NMTN-06058 | SAV0345 | | 390984 | 392699 | 391860 | | |
| NMTN-09219 | SAV0345 | | 390984 | 392699 | 392262 | | |
| NMTN-11735 | SAV0345 | | 390984 | 392699 | 392275 | | |
| NMTN-05694 | SAV0345 | | 390984 | 392699 | 392466 | | |
| NMTN-03885 | SAV0345 | | 390984 | 392699 | 392481 | | |
| NMTN-03887 | SAV0345 | | 390984 | 392699 | 392481 | | |
| NMTN-14491 | SAV0345 | | 390984 | 392699 | 392484 | | |
| NMTN-14795 | SAV0345 | | 390984 | 392699 | 392484 | | |
| NMTN-14801 | SAV0345 | | 390984 | 392699 | 392484 | | |
| NMTN-14803 | SAV0345 | | 390984 | 392699 | 392484 | | |
| NMTN-14811 | SAV0345 | | 390984 | 392699 | 392484 | | |
| NMTN-14836 | SAV0345 | | 390984 | 392699 | 392484 | | |
| NMTN-14849 | SAV0345 | | 390984 | 392699 | 392484 | | |
| NMTN-14853 | SAV0345 | | 390984 | 392699 | 392484 | | |
| NMTN-14887 | SAV0345 | | 390984 | 392699 | 392484 | | |
| NMTN-14915 | SAV0345 | | 390984 | 392699 | 392484 | | |
| NMTN-14921 | SAV0345 | | 390984 | 392699 | 392484 | | |
| NMTN-15053 | SAV0345 | | 390984 | 392699 | 392484 | | |
| NMTN-15007 | SAV0345 | | 390984 | 392699 | 392500 | | |
| NMTN-10082 | SAV0345 | | 390984 | 392699 | 392515 | | |
| NMTN-09556 | SAV0345 | | 390984 | 392699 | 392617 | | |
| NMTN-12868 | SAV0345 | | 390984 | 392699 | 392683 | | |
| NMTN-12780 | SAV0346 | | 392940 | 393602 | 393297 | − | conserved hypothetical protein |
| NMTN-03851 | SAV0346 | | 392940 | 393602 | 393348 | | |
| NMTN-05420 | SAV0346 | | 392940 | 393602 | 393348 | | |
| NMTN-10661 | SAV0348 | | 393981 | 394370 | 394066 | − | hypothetical protein |
| NMTN-10814 | SAV0348 | | 393981 | 394370 | 394363 | | |
| NMTN-14614 | SAV0350 | | 394810 | 395523 | 394925 | + | hypothetical protein |
| NMTN-11890 | SAV0350 | | 394810 | 395523 | 395426 | | |
| NMTN-11900 | SAV0350 | | 394810 | 395523 | 395426 | | |
| NMTN-08896 | SAV0350 | | 394810 | 395523 | 395461 | | |
| NMTN-07402 | SAV0351 | | 395548 | 396390 | 395752 | + | hypothetical protein |
| NMTN-09288 | SAV0351 | | 395548 | 396390 | 395785 | | |
| NMTN-04881 | SAV0351 | | 395548 | 396390 | 395788 | | |
| NMTN-06556 | SAV0352 | | 396390 | 397019 | 396581 | + | conserved hypothetical protein |
| NMTN-12698 | SAV0352 | | 396390 | 397019 | 396831 | | |
| NMTN-12079 | SAV0352 | | 396390 | 397019 | 396866 | | |
| NMTN-03635 | SAV0352 | | 396390 | 397019 | 396869 | | |
| NMTN-03562 | SAV0353 | | 397406 | 398539 | 397622 | − | hypothetical protein |
| NMTN-04172 | SAV0353 | | 397406 | 398539 | 397640 | | |
| NMTN-11979 | SAV0353 | | 397406 | 398539 | 397650 | | |
| NMTN-05351 | SAV0353 | | 397406 | 398539 | 397845 | | |
| NMTN-05352 | SAV0353 | | 397406 | 398539 | 397845 | | |
| NMTN-05421 | SAV0353 | | 397406 | 398539 | 397845 | | |
| NMTN-06533 | SAV0353 | | 397406 | 398539 | 397996 | | |
| NMTN-02127 | SAV0353 | | 397406 | 398539 | 398257 | | |
| NMTN-08839d | SAV0354 | | 399078 | 400259 | 399664 | + | acetyl-CoA C-acetyltransferase |
| | | | | | | | homologue |
| NMTN-09086 | SAV0354 | | 399078 | 400259 | 399930 | | |
| NMTN-10526 | SAV0355 | | 400342 | 401094 | 400373 | − | conserved hypothetical protein |
| NMTN-03134 | SAV0355 | | 400342 | 401094 | 400509 | | |
| NMTN-06551 | SAV0355 | | 400342 | 401094 | 400600 | | |
| NMTN-11313 | SAV0355 | | 400342 | 401094 | 400912 | | |
| NMTN-04642 | SAV0356 | metE | 401137 | 403365 | 401477 | − | 5-methyltetrahydropteroyltriglutamate- |
| | | | | | | | homocysteine methyltransferase |
| NMTN-00724 | SAV0356 | metE | 401137 | 403365 | 402278 | | |
| NMTN-02141 | SAV0356 | metE | 401137 | 403365 | 403012 | | |
| NMTN-00735 | SAV0356 | metE | 401137 | 403365 | 403019 | | |
| NMTN-00167 | SAV0357 | | 403362 | 405203 | 403823 | − | conserved hypothetical protein |
| NMTN-00146 | SAV0357 | | 403362 | 405203 | 403825 | | |
| NMTN-00204 | SAV0357 | | 403362 | 405203 | 404047 | | |
| NMTN-00486 | SAV0357 | | 403362 | 405203 | 404047 | | |
| NMTN-02989 | SAV0357 | | 403362 | 405203 | 404905 | | |
| NMTN-13452 | SAV0357 | | 403362 | 405203 | 404907 | | |
| NMTN-12307 | SAV0358 | | 405172 | 406332 | 405483 | − | hypothetical protein |
| NMTN-12166 | SAV0358 | | 405172 | 406332 | 405543 | | |
| NMTN-08730 | SAV0358 | | 405172 | 406332 | 405818 | | |
| NMTN-12463 | SAV0359 | | 406329 | 407432 | 406927 | − | hypothetical protein |
| NMTN-04976 | SAV0359 | | 406329 | 407432 | 407150 | | |
| NMTN-07988 | SAV0360 | | 408103 | 408939 | 408745 | + | hypothetical protein |
| NMTN-06558 | SAV0360 | | 408103 | 408939 | 408787 | | |
| NMTN-09137 | SAV0360 | | 408103 | 408939 | 408819 | | |
| NMTN-03281 | SAV0360 | | 408103 | 408939 | 408839 | | |
| NMTN-11678 | SAV0361 | | 409093 | 409974 | 409757 | + | conserved hypothetical protein |
| NMTN-14769 | SAV0363 | | 410219 | 411316 | 410351 | + | hypothetical protein |
| NMTN-12209 | SAV0363 | | 410219 | 411316 | 410601 | | |
| NMTN-06441 | SAV0363 | | 410219 | 411316 | 410877 | | |
| NMTN-06449 | SAV0363 | | 410219 | 411316 | 410967 | | |
| NMTN-13266 | SAV0368 | | 413228 | 414202 | 413859 | − | hypothetical protein |
| NMTN-07510 | SAV0368 | | 413228 | 414202 | 413968 | | |
| NMTN-01611 | SAV0368 | | 413228 | 414202 | 413980 | | |
| NMTN-14444 | SAV0368 | | 413228 | 414202 | 413986 | | |
| NMTN-13684 | SAV0369 | | 414254 | 414508 | 415118 | − | truncated integrase |
| NMTN-13684 | SAV0370 | | 415124 | 415735 | 415118 | + | hypothetical protein |
| NMTN-08152 | SAV0370 | | 415124 | 415735 | 415475 | | |
| NMTN-13296 | SAV0370 | | 415124 | 415735 | 415521 | | |
| NMTN-09501 | SAV0370 | | 415124 | 415735 | 415622 | | |
| NMTN-02822 | SAV0372 | | 416653 | 417225 | 416654 | + | conserved hypothetical protein |
| NMTN-07268 | SAV0375 | | 418215 | 418424 | 418275 | − | hypothetical protein |
| NMTN-06108 | SAV0376 | | 418578 | 419183 | 418686 | + | hypothetical protein |
| NMTN-14613d | SAV0376 | | 418578 | 419183 | 418686 | | |
| NMTN-03854 | SAV0376 | | 418578 | 419183 | 418767 | | |
| NMTN-05334 | SAV0376 | | 418578 | 419183 | 419004 | | |
| NMTN-03425 | SAV0377 | | 419249 | 419632 | 419456 | − | conserved hypothetical protein |
| NMTN-04161 | SAV0377 | | 419249 | 419632 | 419457 | | |
| NMTN-01781 | SAV0377 | | 419249 | 419632 | 419460 | | |
| NMTN-01991 | SAV0377 | | 419249 | 419632 | 419494 | | |
| NMTN-15342 | SAV0377 | | 419249 | 419632 | 419496 | | |
| NMTN-12266 | SAV0378 | | 419902 | 420528 | 420434 | − | hypothetical protein |
| NMTN-11517 | SAV0378 | | 419902 | 420528 | 420477 | | |
| NMTN-01746 | SAV0379 | | 420601 | 420792 | 420716 | − | hypothetical protein |
| NMTN-12691 | SAV0381 | ahpC | 422511 | 423080 | 422638 | − | alkyl hydroperoxide reductase subunit C |
| NMTN-14050 | SAV0381 | ahpC | 422511 | 423080 | 422726 | | |
| NMTN-04519 | SAV0382 | | 423572 | 424327 | 423587 | + | hypothetical protein |
| NMTN-05427 | SAV0382 | | 423572 | 424327 | 423589 | | |
| NMTN-13511 | SAV0382 | | 423572 | 424327 | 424030 | | |
| NMTN-06054 | SAV0382 | | 423572 | 424327 | 424128 | | |
| NMTN-05489 | SAV0383 | | 424407 | 425795 | 424649 | − | hypothetical protein |
| NMTN-09471 | SAV0383 | | 424407 | 425795 | 425418 | | |
| NMTN-12236 | SAV0385 | | 428293 | 429249 | 429061 | − | conserved hypothetical protein |
| NMTN-09826 | SAV0386 | | 429367 | 430029 | 429401 | − | hypothetical protein |
| NMTN-09238 | SAV0388 | xprT | 431092 | 431670 | 431101 | + | xanthine phosphoribosyltransferase |
| NMTN-08834 | SAV0389 | pbuX | 431670 | 432938 | 431725 | + | xanthine permease |
| NMTN-07938 | SAV0389 | pbuX | 431670 | 432938 | 431798 | | |
| NMTN-04691 | SAV0389 | pbuX | 431670 | 432938 | 432259 | | |
| NMTN-14620 | SAV0389 | pbuX | 431670 | 432938 | 432765 | | |
| NMTN-02868 | SAV0390 | guaB | 432976 | 434442 | 433027 | + | inositol-monophosphate dehydrogenase |
| NMTN-06706 | SAV0391 | guaA | 434467 | 436008 | 435130 | + | GMP synthase |
| NMTN-02197 | SAV0391 | guaA | 434467 | 436008 | 435193 | | |
| NMTN-10996 | | set21 | 435077 | 435775 | 435373 | | |
| NMTN-15177 | SAV0391 | guaA | 434467 | 436008 | 435451 | | |
| NMTN-11946 | SAV0391 | guaA | 434467 | 436008 | 435583 | | |
| NMTN-06940 | SAV0391 | guaA | 434467 | 436008 | 435587 | | |
| NMTN-08349 | SAV0391 | guaA | 434467 | 436008 | 435619 | | |
| NMTN-11810 | SAV0391 | guaA | 434467 | 436008 | 435940 | | |
| NMTN-10413 | | set24 | 438308 | 439006 | 438550 | | |
| NMTN-10224 | SAV0416 | | 461922 | 462458 | 462194 | − | hypothetical protein |
| NMTN-06701 | SAV0417 | | 462850 | 463554 | 462954 | − | hypothetical protein |
| NMTN-09364 | SAV0417 | | 462850 | 463554 | 462988 | | |
| NMTN-11889 | SAV0417 | | 462850 | 463554 | 463489 | | |
| NMTN-11897 | SAV0417 | | 462850 | 463554 | 463492 | | |
| NMTN-11908 | SAV0417 | | 462850 | 463554 | 463516 | | |
| NMTN-00572 | SAV0420 | | 465170 | 465529 | 465329 | − | conserved hypothetical protein |
| NMTN-11141 | SAV0420 | | 465170 | 465529 | 465471 | | |
| NMTN-06130 | SAV0421 | | 465548 | 466393 | 465927 | − | conserved hypothetical protein |
| NMTN-12619 | SAV0421 | | 465548 | 466393 | 465984 | | |
| NMTN-09567 | SAV0424 | set8 | 468817 | 469887 | 469438 | + | exotoxin 8 |
| NMTN-04681 | SAV0424 | set8 | 468817 | 469887 | 469491 | | |
| NMTN-10262 | SAV0424 | set8 | 468817 | 469887 | 469674 | | |
| NMTN-10490 | SAV0424 | set8 | 468817 | 469887 | 469867 | | |
| NMTN-08867 | SAV0425 | set10 | 470251 | 470955 | 470290 | + | exotoxin 10 |
| NMTN-09122 | SAV0425 | set10 | 470251 | 470955 | 470335 | | |
| NMTN-13316 | SAV0425 | set10 | 470251 | 470955 | 470471 | | |
| NMTN-11488 | SAV0425 | set10 | 470251 | 470955 | 470512 | | |
| NMTN-12345 | SAV0425 | set10 | 470251 | 470955 | 470563 | | |
| NMTN-09645 | SAV0425 | set10 | 470251 | 470955 | 470644 | | |
| NMTN-03859 | SAV0426 | set11 | 471401 | 472096 | 471521 | + | exotoxin 11 |
| NMTN-15240 | SAV0426 | set11 | 471401 | 472096 | 471521 | | |
| NMTN-11318 | SAV0426 | set11 | 471401 | 472096 | 471528 | | |
| NMTN-03905 | SAV0426 | set11 | 471401 | 472096 | 471603 | | |
| NMTN-12292 | SAV0426 | set11 | 471401 | 472096 | 472097 | | |
| NMTN-02183 | SAV0427 | set12 | 472435 | 473133 | 472453 | + | exotoxin 12 |
| NMTN-07136 | SAV0427 | set12 | 472435 | 473133 | 472474 | | |
| NMTN-01980 | SAV0427 | set12 | 472435 | 473133 | 472855 | | |
| NMTN-01645 | SAV0428 | set13 | 473510 | 474208 | 473559 | + | exotoxin 13 |
| NMTN-10409 | SAV0428 | set13 | 473510 | 474208 | 473754 | | |
| NMTN-10261 | SAV0428 | set13 | 473510 | 474208 | 473758 | | |
| NMTN-09836 | SAV0428 | set13 | 473510 | 474208 | 473791 | | |
| NMTN-03059 | SAV0428 | set13 | 473510 | 474208 | 473909 | | |
| NMTN-06098 | SAV0428 | set13 | 473510 | 474208 | 474153 | | |
| NMTN-12470 | SAV0429 | set14 | 474574 | 475257 | 475177 | + | exotoxin 14 |
| NMTN-11723 | SAV0429 | set14 | 474574 | 475257 | 475202 | | |
| NMTN-06340 | SAV0430 | | 475310 | 475468 | 475404 | + | hypothetical protein |
| NMTN-14715 | SAV0432 | hsdS | 477070 | 478281 | 477173 | + | probable restriction modification system |
| | | | | | | | specificity subunit |
| NMTN-09115 | SAV0432 | hsdS | 477070 | 478281 | 477821 | | |
| NMTN-06674 | SAV0432 | hsdS | 477070 | 478281 | 477988 | | |
| NMTN-00606 | SAV0432 | hsdS | 477070 | 478281 | 477989 | | |
| NMTN-11974 | SAV0432 | hsdS | 477070 | 478281 | 478095 | | |
| NMTN-14438 | SAV0432 | hsdS | 477070 | 478281 | 478137 | | |
| NMTN-00661 | SAV0432 | hsdS | 477070 | 478281 | 478144 | | |
| NMTN-04805 | SAV0432 | hsdS | 477070 | 478281 | 478156 | | |
| NMTN-13910 | SAV0433 | set15 | 478664 | 479347 | 478806 | + | exotoxin 15 |
| NMTN-01618 | SAV0434 | | 479369 | 480856 | 479411 | + | hypothetical protein |
| NMTN-09712 | SAV0434 | | 479369 | 480856 | 480149 | | |
| NMTN-08110 | SAV0434 | | 479369 | 480856 | 480357 | | |
| NMTN-00801 | SAV0434 | | 479369 | 480856 | 480560 | | |
| NMTN-01996 | SAV0436 | lpl1 | 481609 | 482412 | 481781 | + | hypothetical protein |
| NMTN-00586 | SAV0438 | lpl3 | 483282 | 484070 | 483318 | + | hypothetical protein |
| NMTN-12927 | SAV0441 | lpl5 | 485741 | 486523 | 485973 | + | hypothetical protein |
| NMTN-12150 | SAV0441 | lpl5 | 485741 | 486523 | 486170 | | |
| NMTN-12268 | SAV0442 | lpl6 | 486580 | 487374 | 486600 | + | hypothetical protein |
| NMTN-06425 | SAV0444 | lpl8 | 488245 | 489027 | 488272 | + | hypothetical protein |
| NMTN-04371 | SAV0444 | lpl8 | 488245 | 489027 | 488474 | | |
| NMTN-00383 | SAV0445 | lpl9 | 489206 | 490021 | 489375 | + | hypothetical protein |
| NMTN-12829 | SAV0446 | | 490011 | 491336 | 490395 | + | hypothetical protein |
| NMTN-05045 | SAV0446 | | 490011 | 491336 | 490632 | | |
| NMTN-01489 | SAV0446 | | 490011 | 491336 | 490705 | | |
| NMTN-02862 | SAV0446 | | 490011 | 491336 | 490803 | | |
| NMTN-12950 | SAV0446 | | 490011 | 491336 | 490899 | | |
| NMTN-12452 | SAV0446 | | 490011 | 491336 | 491106 | | |
| NMTN-04779 | SAV0446 | | 490011 | 491336 | 491173 | | |
| NMTN-02881 | SAV0446 | | 490011 | 491336 | 491181 | | |
| NMTN-03660 | SAV0446 | | 490011 | 491336 | 491319 | | |
| NMTN-02829 | SAV0448 | | 491675 | 491989 | 491719 | + | hypothetical protein |
| NMTN-05251 | SAV0449 | | 492278 | 492478 | 492373 | − | hypothetical protein |
| NMTN-07283 | SAV0450 | | 492657 | 493859 | 492653 | + | conserved hypothetical protein |
| NMTN-09060 | SAV0450 | | 492657 | 493859 | 492656 | | |
| NMTN-04173 | SAV0450 | | 492657 | 493859 | 492786 | | |
| NMTN-10015 | SAV0450 | | 492657 | 493859 | 493185 | | |
| NMTN-05506 | SAV0450 | | 492657 | 493859 | 493450 | | |
| NMTN-03754 | SAV0450 | | 492657 | 493859 | 493563 | | |
| NMTN-00537 | SAV0455 | | 500402 | 500644 | 500437 | − | hypothetical protein |
| NMTN-10904 | SAV0455 | | 500402 | 500644 | 500534 | | |
| NMTN-03741 | SAV0456 | | 500856 | 501530 | 501167 | + | conserved hypothetical protein |
| NMTN-02151 | SAV0456 | | 500856 | 501530 | 501216 | | |
| NMTN-02530 | SAV0456 | | 500856 | 501530 | 501233 | | |
| NMTN-05316 | SAV0457 | | 501567 | 502301 | 501630 | + | hypothetical protein |
| NMTN-00687 | SAV0457 | | 501567 | 502301 | 501707 | | |
| NMTN-12744 | SAV0457 | | 501567 | 502301 | 501755 | | |
| NMTN-09422 | SAV0457 | | 501567 | 502301 | 501765 | | |
| NMTN-06560 | SAV0457 | | 501567 | 502301 | 502038 | | |
| NMTN-01138 | SAV0458 | | 502670 | 504010 | 503125 | + | hypothetical protein |
| NMTN-11558 | SAV0458 | | 502670 | 504010 | 504013 | | |
| NMTN-14431 | SAV0459 | cysM | 504227 | 505132 | 504411 | + | cysteine synthase homologue |
| NMTN-04955 | SAV0459 | cysM | 504227 | 505132 | 504480 | | |
| NMTN-06894 | SAV0459 | cysM | 504227 | 505132 | 504670 | | |
| NMTN-12011 | SAV0459 | cysM | 504227 | 505132 | 504670 | | |
| NMTN-03815 | SAV0459 | cysM | 504227 | 505132 | 504675 | | |
| NMTN-09274 | SAV0459 | cysM | 504227 | 505132 | 504983 | | |
| NMTN-03966 | SAV0459 | cysM | 504227 | 505132 | 505086 | | |
| NMTN-08429 | SAV0459 | cysM | 504227 | 505132 | 505087 | | |
| NMTN-06902 | SAV0461 | yrhB | 505146 | 506266 | 505198 | | |
| NMTN-03554 | SAV0461 | yrhB | 505146 | 506266 | 505213 | | |
| NMTN-00099 | SAV0461 | yrhB | 505146 | 506266 | 505221 | + | cystathionine gamma-synthase homolog |
| NMTN-04796 | SAV0461 | yrhB | 505146 | 506266 | 505486 | | |
| NMTN-01861 | SAV0461 | yrhB | 505146 | 506266 | 505725 | | |
| NMTN-09549 | SAV0462 | | 506561 | 507586 | 506603 | + | hypothetical protein |
| NMTN-00841 | SAV0462 | | 506561 | 507586 | 506778 | | |
| NMTN-11775 | SAV0463 | | 507590 | 508249 | 507845 | + | hypothetical protein |
| NMTN-08336 | SAV0463 | | 507590 | 508249 | 507941 | | |
| NMTN-02295 | SAV0463 | | 507590 | 508249 | 508003 | | |
| NMTN-03585 | SAV0463 | | 507590 | 508249 | 508129 | | |
| NMTN-05733 | SAV0463 | | 507590 | 508249 | 508181 | | |
| NMTN-01857 | SAV0464 | | 508286 | 509128 | 508325 | + | hypothetical protein |
| NMTN-13917 | SAV0464 | | 508286 | 509128 | 508547 | | |
| NMTN-00339 | SAV0464 | | 508286 | 509128 | 508770 | | |
| NMTN-09977 | SAV0464 | | 508286 | 509128 | 508910 | | |
| NMTN-05539 | SAV0464 | | 508286 | 509128 | 508947 | | |
| NMTN-09353 | SAV0465 | | 509460 | 510263 | 509835 | + | hypothetical protein |
| NMTN-11802 | SAV0465 | | 509460 | 510263 | 509947 | | |
| NMTN-01696 | SAV0465 | | 509460 | 510263 | 509950 | | |
| NMTN-12140 | SAV0465 | | 509460 | 510263 | 510007 | | |
| NMTN-04405 | SAV0465 | | 509460 | 510263 | 510090 | | |
| NMTN-08084 | SAV0466 | | 510448 | 510726 | 510435 | − | hypothetical protein |
| NMTN-14425 | SAV0466 | | 510448 | 510726 | 510625 | | |
| NMTN-06582 | SAV0466 | | 510448 | 510726 | 510729 | | |
| NMTN-02115 | SAV0467 | | 510866 | 511261 | 511172 | + | conserved hypothetical protein |
| NMTN-09534 | SAV0467 | | 510866 | 511261 | 511258 | | |
| NMTN-09534 | SAV0468 | | 511251 | 511736 | 511258 | + | conserved hypothetical protein |
| NMTN-00464 | SAV0468 | | 511251 | 511736 | 511676 | | |
| NMTN-08141 | SAV0469 | | 511905 | 512687 | 512096 | − | conserved hypothetical protein |
| NMTN-03852 | SAV0469 | | 511905 | 512687 | 512248 | | |
| NMTN-04062 | SAV0469 | | 511905 | 512687 | 512561 | | |
| NMTN-04068 | SAV0469 | | 511905 | 512687 | 512561 | | |
| NMTN-04070 | SAV0469 | | 511905 | 512687 | 512561 | | |
| NMTN-04131 | SAV0470 | | 512684 | 513796 | 512813 | − | conserved hypothetical protein |
| NMTN-05165 | SAV0470 | | 512684 | 513796 | 512856 | | |
| NMTN-03883 | SAV0470 | | 512684 | 513796 | 513127 | | |
| NMTN-06596 | SAV0470 | | 512684 | 513796 | 513382 | | |
| NMTN-11576 | SAV0470 | | 512684 | 513796 | 513561 | | |
| NMTN-12685 | SAV0471 | gltC | 513917 | 514801 | 514401 | − | transcription activator of glutamate |
| | | | | | | | synthase operon |
| NMTN-06250 | SAV0471 | gltC | 513917 | 514801 | 514811 | | |
| NMTN-11578 | SAV0472 | gltB | 514982 | 519481 | 515912 | + | glutamate synthase large subunit |
| NMTN-10043 | SAV0472 | gltB | 514982 | 519481 | 516259 | | |
| NMTN-14687 | SAV0472 | gltB | 514982 | 519481 | 516562 | | |
| NMTN-04151 | SAV0472 | gltB | 514982 | 519481 | 516821 | | |
| NMTN-04441 | SAV0472 | gltB | 514982 | 519481 | 517008 | | |
| NMTN-11069 | SAV0472 | gltB | 514982 | 519481 | 517104 | | |
| NMTN-10228 | SAV0472 | gltB | 514982 | 519481 | 517297 | | |
| NMTN-03726 | SAV0472 | gltB | 514982 | 519481 | 517651 | | |
| NMTN-04377 | SAV0472 | gltB | 514982 | 519481 | 517817 | | |
| NMTN-09438 | SAV0472 | gltB | 514982 | 519481 | 517925 | | |
| NMTN-11119 | SAV0472 | gltB | 514982 | 519481 | 518147 | | |
| NMTN-00215 | SAV0472 | gltB | 514982 | 519481 | 518838 | | |
| NMTN-00369 | SAV0472 | gltB | 514982 | 519481 | 518838 | | |
| NMTN-00138 | SAV0472 | gltB | 514982 | 519481 | 519098 | | |
| NMTN-10929 | SAV0472 | gltB | 514982 | 519481 | 519123 | | |
| NMTN-13372 | SAV0472 | gltB | 514982 | 519481 | 519237 | | |
| NMTN-12044 | SAV0473 | gltD | 519499 | 520962 | 519675 | + | NADH-glutamate synthase small subunit |
| NMTN-08561 | SAV0473 | gltD | 519499 | 520962 | 519748 | | |
| NMTN-02133 | SAV0473 | gltD | 519499 | 520962 | 520015 | | |
| NMTN-05001 | SAV0473 | gltD | 519499 | 520962 | 520077 | | |
| NMTN-12442 | SAV0473 | gltD | 519499 | 520962 | 520228 | | |
| NMTN-08530 | SAV0473 | gltD | 519499 | 520962 | 520229 | | |
| NMTN-03677 | SAV0473 | gltD | 519499 | 520962 | 520348 | | |
| NMTN-04723 | SAV0473 | gltD | 519499 | 520962 | 520469 | | |
| NMTN-00072 | SAV0474 | treP | 521792 | 523219 | 522098 | + | phosphoenolpyruvate-dependent and |
| | | | | | | | trehalose-specific PTS enzyme II |
| NMTN-14632 | SAV0474 | treP | 521792 | 523219 | 522808 | | |
| NMTN-09541 | SAV0474 | treP | 521792 | 523219 | 522941 | | |
| NMTN-14604 | SAV0474 | treP | 521792 | 523219 | 523117 | | |
| NMTN-03871 | SAV0475 | | 523283 | 524923 | 523899 | + | alpha-glucosidase |
| NMTN-12360 | SAV0476 | | 524948 | 525676 | 525471 | + | hypothetical protein |
| NMTN-03557 | SAV0476 | | 524948 | 525676 | 525541 | | |
| NMTN-11167 | SAV0481 | | 536410 | 537747 | 536996 | + | hypothetical protein |
| NMTN-13561 | SAV0481 | | 536410 | 537747 | 537107 | | |
| NMTN-07048 | SAV0481 | | 536410 | 537747 | 537306 | | |
| NMTN-15220d | SAV0481 | | 536410 | 537747 | 537524 | | |
| NMTN-12986 | SAV0481 | | 536410 | 537747 | 537528 | | |
| NMTN-14049 | SAV0485 | | 540032 | 540667 | 540183 | + | hypothetical protein |
| NMTN-03926 | SAV0487 | | 541305 | 542030 | 541504 | + | conserved hypothetical protein |
| NMTN-01943 | SAV0487 | | 541305 | 542030 | 541776 | | |
| NMTN-11308 | SAV0487 | | 541305 | 542030 | 541879 | | |
| NMTN-01612 | SAV0488 | | 542023 | 542271 | 542072 | + | conserved hypothetical protein |
| NMTN-10845 | SAV0488 | | 542023 | 542271 | 542075 | | |
| NMTN-04831 | SAV0489 | | 542273 | 543112 | 542650 | + | conserved hypothetical protein |
| NMTN-05607 | SAV0489 | | 542273 | 543112 | 543086 | | |
| NMTN-13072 | SAV0489 | | 542273 | 543112 | 543107 | | |
| NMTN-05397 | SAV0490 | metS | 543397 | 545370 | 545072 | + | methionyl-tRNA synthetase |
| NMTN-01766 | SAV0490 | metS | 543397 | 545370 | 545075 | | |
| NMTN-03488 | SAV0492 | | 546341 | 546877 | 546337 | + | conserved hypothetical protein |
| NMTN-06122 | SAV0493 | ksgA | 546882 | 547772 | 547143 | + | dimethyladenosine transferase |
| NMTN-05534 | SAV0493 | ksgA | 546882 | 547772 | 547452 | | |
| NMTN-08899d | SAV0495 | | 548453 | 549301 | 548875 | + | hypothetical protein |
| NMTN-09720 | SAV0499 | gcaD | 551317 | 552669 | 552649 | + | UDP-N-acetylglucosamine |
| | | | | | | | pyrophosphorylase homologue |
| NMTN-11929 | SAV0501 | rplY | 553931 | 554584 | 554021 | + | 50S ribosomal protein L25 |
| NMTN-09944 | SAV0501 | rplY | 553931 | 554584 | 554061 | | |
| NMTN-10050 | SAV0501 | rplY | 553931 | 554584 | 554061 | | |
| NMTN-02410 | SAV0501 | rplY | 553931 | 554584 | 554315 | | |
| NMTN-09430 | SAV0503 | mfd | 555467 | 558973 | 555683 | + | transcription-repair coupling factor |
| NMTN-10525 | SAV0503 | mfd | 555467 | 558973 | 557549 | | |
| NMTN-11860 | SAV0503 | mfd | 555467 | 558973 | 558256 | | |
| NMTN-11483 | SAV0504 | | 558963 | 560489 | 559588 | + | hypothetical protein |
| NMTN-04818 | SAV0511 | ftsH | 565134 | 567227 | 565240 | + | cell-division protein |
| NMTN-13644 | SAV0511 | ftsH | 565134 | 567227 | 565240 | | |
| NMTN-11982 | SAV0511 | ftsH | 565134 | 567227 | 567103 | | |
| NMTN-07872 | SAV0512 | | 567455 | 568336 | 567440 | + | heat-shock protein HSP33 homologue |
| NMTN-07334 | SAV0512 | | 567455 | 568336 | 567504 | | |
| NMTN-04925 | SAV0512 | | 567455 | 568336 | 567721 | | |
| NMTN-06938 | SAV0512 | | 567455 | 568336 | 567838 | | |
| NMTN-06790 | SAV0514 | folP | 569663 | 570466 | 569694 | + | dihydropteroate synthase chain A |
| | | | | | | | synthetase |
| NMTN-02416 | SAV0516 | folK | 570806 | 571282 | 571275 | + | 2-amino-4-hydroxy-6- |
| | | | | | | | hydroxymethyldihydropteridine |
| | | | | | | | pyrophosphokinase |
| NMTN-09310 | SAV0516 | folK | 570806 | 571282 | 571282 | | |
| NMTN-09568 | SAV0516 | folK | 570806 | 571282 | 571282 | | |
| NMTN-10020 | SAV0518 | | 580361 | 581743 | 580545 | − | hypothetical protein |
| NMTN-10828 | SAV0518 | | 580361 | 581743 | 580563 | | |
| NMTN-13540 | SAV0518 | | 580361 | 581743 | 580876 | | |
| NMTN-06053 | SAV0518 | | 580361 | 581743 | 580879 | | |
| NMTN-09910 | SAV0518 | | 580361 | 581743 | 580944 | | |
| NMTN-07770 | SAV0518 | | 580361 | 581743 | 581042 | | |
| NMTN-09862 | SAV0518 | | 580361 | 581743 | 581148 | | |
| NMTN-10489 | SAV0518 | | 580361 | 581743 | 581402 | | |
| NMTN-09746 | SAV0518 | | 580361 | 581743 | 581427 | | |
| NMTN-04532 | SAV0518 | | 580361 | 581743 | 581438 | | |
| NMTN-10482 | SAV0518 | | 580361 | 581743 | 581496 | | |
| NMTN-03216 | SAV0518 | | 580361 | 581743 | 581692 | | |
| NMTN-15271d | SAV0519 | | 581847 | 582734 | 581845 | + | conserved hypothetical protein |
| NMTN-00222 | SAV0519 | | 581847 | 582734 | 582028 | | |
| NMTN-07952 | SAV0520 | | 582738 | 583298 | 582823 | + | conserved hypothetical protein |
| NMTN-14498 | SAV0520 | | 582738 | 583298 | 583135 | | |
| NMTN-10259 | SAV0520 | | 582738 | 583298 | 583154 | | |
| NMTN-08330 | SAV0520 | | 582738 | 583298 | 583170 | | |
| NMTN-09357 | SAV0520 | | 582738 | 583298 | 583170 | | |
| NMTN-13570 | SAV0520 | | 582738 | 583298 | 583273 | | |
| NMTN-01480 | SAV0521 | nupC | 583506 | 584720 | 583717 | − | pyrimidine nucleoside transport protein |
| NMTN-08491 | SAV0521 | nupC | 583506 | 584720 | 583717 | | |
| NMTN-03483 | SAV0521 | nupC | 583506 | 584720 | 584083 | | |
| NMTN-04483 | SAV0521 | nupC | 583506 | 584720 | 584420 | | |
| NMTN-14043 | SAV0521 | nupC | 583506 | 584720 | 584615 | | |
| NMTN-00696 | SAV0522 | ctsR | 584878 | 585339 | 584888 | + | transcription repressor of class III stress |
| | | | | | | | genes homologue |
| NMTN-02037 | SAV0523 | | 585358 | 585924 | 585562 | + | conserved hypothetical protein |
| NMTN-12880 | SAV0523 | | 585358 | 585924 | 585710 | | |
| NMTN-11531 | SAV0524 | | 585914 | 586921 | 586497 | + | hypothetical protein |
| NMTN-11228 | SAV0525 | clpC | 586935 | 589391 | 587758 | + | endopeptidase |
| NMTN-03019 | SAV0525 | clpC | 586935 | 589391 | 588453 | | |
| NMTN-00446 | SAV0525 | clpC | 586935 | 589391 | 588760 | | |
| NMTN-12647 | SAV0526 | radA | 589876 | 591240 | 590057 | + | DNA repair protein homologue |
| NMTN-03634 | SAV0526 | radA | 589876 | 591240 | 590235 | | |
| NMTN-09979 | SAV0526 | radA | 589876 | 591240 | 590887 | | |
| NMTN-05910 | SAV0527 | | 591265 | 592338 | 591394 | + | conserved hypothetical protein |
| NMTN-00437 | SAV0527 | | 591265 | 592338 | 591397 | | |
| NMTN-07475 | SAV0527 | | 591265 | 592338 | 591408 | | |
| NMTN-12850 | SAV0527 | | 591265 | 592338 | 591467 | | |
| NMTN-11391 | SAV0527 | | 591265 | 592338 | 591934 | | |
| NMTN-00666 | SAV0527 | | 591265 | 592338 | 591955 | | |
| NMTN-11689 | SAV0528 | gltX | 592895 | 594349 | 594345 | + | glutamyl-tRNA synthetase |
| NMTN-12388 | SAV0528 | gltX | 592895 | 594349 | 594349 | | |
| NMTN-12390 | SAV0528 | gltX | 592895 | 594349 | 594349 | | |
| NMTN-12391 | SAV0528 | gltX | 592895 | 594349 | 594349 | | |
| NMTN-12400 | SAV0528 | gltX | 592895 | 594349 | 594349 | | |
| NMTN-12403 | SAV0528 | gltX | 592895 | 594349 | 594349 | | |
| NMTN-07942 | SAV0530 | cysS | 595373 | 597199 | 596799 | + | cysteinyl-tRNA synthetase |
| NMTN-12176 | SAV0530 | cysS | 595373 | 597199 | 596887 | | |
| NMTN-02860 | SAV0530 | cysS | 595373 | 597199 | 596951 | | |
| NMTN-00708 | SAV0531 | | 597207 | 597953 | 597504 | + | hypothetical protein |
| NMTN-13498 | SAV0531 | | 597207 | 597953 | 597870 | | |
| NMTN-11988 | SAV0532 | | 597953 | 598477 | 598214 | + | conserved hypothetical protein |
| NMTN-03612 | SAV0541 | | 603045 | 603653 | 603505 | + | conserved hypothetical protein |
| NMTN-13622 | SAV0541 | | 603045 | 603653 | 603640 | | |
| NMTN-03510 | SAV0549 | | 616504 | 617679 | 616617 | − | hypothetical protein |
| NMTN-13208 | SAV0549 | | 616504 | 617679 | 616774 | | |
| NMTN-04952 | SAV0549 | | 616504 | 617679 | 616889 | | |
| NMTN-12942 | SAV0549 | | 616504 | 617679 | 616919 | | |
| NMTN-03459 | SAV0549 | | 616504 | 617679 | 617639 | | |
| NMTN-09539 | SAV0550 | | 617886 | 619037 | 617924 | + | hypothetical protein |
| NMTN-03858 | SAV0550 | | 617886 | 619037 | 618380 | | |
| NMTN-06156 | SAV0550 | | 617886 | 619037 | 618403 | | |
| NMTN-13497 | SAV0551 | | 619307 | 620185 | 619607 | + | conserved hypothetical protein |
| NMTN-06526 | SAV0551 | | 619307 | 620185 | 620040 | | |
| NMTN-10434 | SAV0551 | | 619307 | 620185 | 620119 | | |
| NMTN-01604 | SAV0551 | | 619307 | 620185 | 620162 | | |
| NMTN-09479 | SAV0552 | araB | 620344 | 621981 | 620447 | + | probable L-ribulokinase |
| NMTN-04399 | SAV0552 | araB | 620344 | 621981 | 620492 | | |
| NMTN-02892 | SAV0552 | araB | 620344 | 621981 | 620495 | | |
| NMTN-09249 | SAV0552 | araB | 620344 | 621981 | 620552 | | |
| NMTN-09252 | SAV0552 | araB | 620344 | 621981 | 620552 | | |
| NMTN-09253 | SAV0552 | araB | 620344 | 621981 | 620552 | | |
| NMTN-09255 | SAV0552 | araB | 620344 | 621981 | 620552 | | |
| NMTN-09256 | SAV0552 | araB | 620344 | 621981 | 620552 | | |
| NMTN-09258 | SAV0552 | araB | 620344 | 621981 | 620552 | | |
| NMTN-09260 | SAV0552 | araB | 620344 | 621981 | 620552 | | |
| NMTN-09261 | SAV0552 | araB | 620344 | 621981 | 620552 | | |
| NMTN-09262 | SAV0552 | araB | 620344 | 621981 | 620552 | | |
| NMTN-09273 | SAV0552 | araB | 620344 | 621981 | 620552 | | |
| NMTN-09275 | SAV0552 | araB | 620344 | 621981 | 620552 | | |
| NMTN-08988 | SAV0552 | araB | 620344 | 621981 | 620555 | | |
| NMTN-03229 | SAV0552 | araB | 620344 | 621981 | 620587 | | |
| NMTN-04353 | SAV0552 | araB | 620344 | 621981 | 621001 | | |
| NMTN-06281 | SAV0552 | araB | 620344 | 621981 | 621037 | | |
| NMTN-10454 | SAV0552 | araB | 620344 | 621981 | 621513 | | |
| NMTN-06335 | SAV0552 | araB | 620344 | 621981 | 621567 | | |
| NMTN-10943 | SAV0552 | araB | 620344 | 621981 | 621567 | | |
| NMTN-09259 | SAV0552 | araB | 620344 | 621981 | 621635 | | |
| NMTN-00027 | SAV0552 | araB | 620344 | 621981 | 621772 | | |
| NMTN-07226d | SAV0553 | | 622195 | 623160 | 622298 | + | hypothetical protein |
| NMTN-00610 | SAV0553 | | 622195 | 623160 | 622821 | | |
| NMTN-09073 | SAV0554 | ilvE | 623496 | 624572 | 624025 | + | branched-chain amino acid |
| | | | | | | | aminotroansferase homologue |
| NMTN-08716 | SAV0554 | ilvE | 623496 | 624572 | 624036 | | |
| NMTN-12385 | SAV0554 | ilvE | 623496 | 624572 | 624153 | | |
| NMTN-12251 | SAV0555 | | 624822 | 625505 | 624851 | + | conserved hypothetical protein |
| NMTN-08699 | SAV0555 | | 624822 | 625505 | 625054 | | |
| NMTN-14415 | SAV0555 | | 624822 | 625505 | 625347 | | |
| NMTN-13458 | SAV0555 | | 624822 | 625505 | 625446 | | |
| NMTN-06474 | SAV0556 | | 625621 | 626283 | 625721 | − | hypothetical protein |
| NMTN-07026 | SAV0556 | | 625621 | 626283 | 625790 | | |
| NMTN-03850 | SAV0556 | | 625621 | 626283 | 625861 | | |
| NMTN-02743 | SAV0559 | | 627577 | 628446 | 627647 | | conserved hypothetical protein |
| NMTN-11104 | SAV0559 | | 627577 | 628446 | 627665 | | |
| NMTN-08528 | SAV0559 | | 627577 | 628446 | 628025 | | |
| NMTN-13068d | SAV0560 | | 628468 | 629034 | 628789 | | conserved hypothetical protein |
| NMTN-09013 | SAV0560 | | 628468 | 629034 | 628894 | | |
| NMTN-06677 | SAV0561 | sdrC | 629464 | 632325 | 629786 | + | Ser-Asp rich fibrinogen-binding, bone |
| | | | | | | | sialoprotein-binding protein |
| NMTN-03932 | SAV0561 | sdrC | 629464 | 632325 | 629876 | | |
| NMTN-08654 | SAV0561 | sdrC | 629464 | 632325 | 629933 | | |
| NMTN-00071 | SAV0561 | sdrC | 629464 | 632325 | 630306 | | |
| NMTN-12620 | SAV0561 | sdrC | 629464 | 632325 | 630311 | | |
| NMTN-02927 | SAV0561 | sdrC | 629464 | 632325 | 630325 | | |
| NMTN-06131 | SAV0561 | sdrC | 629464 | 632325 | 630510 | | |
| NMTN-09994 | SAV0561 | sdrC | 629464 | 632325 | 630597 | | |
| NMTN-07414 | SAV0561 | sdrC | 629464 | 632325 | 630639 | | |
| NMTN-10232 | SAV0561 | sdrC | 629464 | 632325 | 630739 | | |
| NMTN-06187 | SAV0561 | sdrC | 629464 | 632325 | 630791 | | |
| NMTN-05174 | SAV0561 | sdrC | 629464 | 632325 | 630899 | | |
| NMTN-06089 | SAV0561 | sdrC | 629464 | 632325 | 630966 | | |
| NMTN-03888 | SAV0561 | sdrC | 629464 | 632325 | 631255 | | |
| NMTN-01536 | SAV0561 | sdrC | 629464 | 632325 | 632024 | | |
| NMTN-00318 | SAV0561 | sdrC | 629464 | 632325 | 632194 | | |
| NMTN-00124 | SAV0561 | sdrC | 629464 | 632325 | 632195 | | |
| NMTN-00255 | SAV0561 | sdrC | 629464 | 632325 | 632195 | | |
| NMTN-09605 | SAV0562 | sdrD | 632692 | 636849 | 633324 | + | Ser-Asp rich fibrinogen-binding, bone |
| | | | | | | | sialoprotein-binding protein |
| NMTN-09045 | SAV0562 | sdrD | 632692 | 636849 | 634409 | | |
| NMTN-05302 | SAV0562 | sdrD | 632692 | 636849 | 634413 | | |
| NMTN-03506 | SAV0562 | sdrD | 632692 | 636849 | 634416 | | |
| NMTN-10396 | SAV0562 | sdrD | 632692 | 636849 | 635413 | | |
| NMTN-11285 | SAV0563 | sdrE | 637243 | 640668 | 637448 | + | Ser-Asp rich fibrinogen-binding, bone |
| | | | | | | | sialoprotein-binding protein |
| NMTN-11826 | SAV0563 | sdrE | 637243 | 640668 | 637465 | | |
| NMTN-09904 | SAV0563 | sdrE | 637243 | 640668 | 637491 | | |
| NMTN-08963 | SAV0563 | sdrE | 637243 | 640668 | 638120 | | |
| NMTN-03489 | SAV0563 | sdrE | 637243 | 640668 | 638339 | | |
| NMTN-00583 | SAV0563 | sdrE | 637243 | 640668 | 638367 | | |
| NMTN-00254 | SAV0563 | sdrE | 637243 | 640668 | 638647 | | |
| NMTN-12588 | SAV0563 | sdrE | 637243 | 640668 | 638707 | | |
| NMTN-01560 | SAV0563 | sdrE | 637243 | 640668 | 638756 | | |
| NMTN-11817 | SAV0563 | sdrE | 637243 | 640668 | 639167 | | |
| NMTN-01448 | SAV0563 | sdrE | 637243 | 640668 | 639335 | | |
| NMTN-04553 | SAV0563 | sdrE | 637243 | 640668 | 639515 | | |
| NMTN-04694 | SAV0563 | sdrE | 637243 | 640668 | 640335 | | |
| NMTN-02509 | SAV0564 | | 640789 | 642261 | 640902 | + | hypothetical protein |
| NMTN-11800 | SAV0564 | | 640789 | 642261 | 640902 | | |
| NMTN-05212 | SAV0564 | | 640789 | 642261 | 640971 | | |
| NMTN-03187 | SAV0564 | | 640789 | 642261 | 641095 | | |
| NMTN-08741 | SAV0564 | | 640789 | 642261 | 641224 | | |
| NMTN-00434 | SAV0564/ | | 640789 | 642261 | 641282 | | |
| | SA1797 | | | | | | |
| NMTN-00800 | SAV0564 | | 640789 | 642261 | 641614 | | |
| NMTN-10480 | SAV0565 | | 642386 | 643876 | 642394 | − | hypothetical protein |
| NMTN-04788 | SAV0565 | | 642386 | 643876 | 643510 | | |
| NMTN-07891 | SAV0565 | | 642386 | 643876 | 643617 | | |
| NMTN-09690 | SAV0567 | | 645049 | 645714 | 645381 | − | conserved hypothetical protein |
| NMTN-04823 | SAV0568 | | 645728 | 646087 | 645924 | − | conserved hypothetical protein |
| NMTN-09062 | SAV0568 | | 645728 | 646087 | 645931 | | |
| NMTN-09065 | SAV0568 | | 645728 | 646087 | 645931 | | |
| NMTN-11650 | SAV0569 | nagB | 646366 | 647124 | 646766 | + | probable glucosamine-6-phosphate |
| | | | | | | | isomerase |
| NMTN-12246 | SAV0570 | | 647201 | 647833 | 647712 | + | hypothetical protein |
| NMTN-03062 | SAV0571 | | 647835 | 648383 | 647949 | + | conserved hypothetical protein |
| NMTN-13759 | SAV0571 | | 647835 | 648383 | 648117 | | |
| NMTN-08820 | SAV0572 | | 648496 | 649143 | 648627 | + | hypothetical protein |
| NMTN-03520 | SAV0572 | | 648496 | 649143 | 648644 | | |
| NMTN-03735 | SAV0572 | | 648496 | 649143 | 648644 | | |
| NMTN-05026 | SAV0572 | | 648496 | 649143 | 648722 | | |
| NMTN-03856 | SAV0573 | proP | 649645 | 651045 | 649913 | + | proline/betaine transporter homologue |
| NMTN-04380 | SAV0573 | proP | 649645 | 651045 | 650079 | | |
| NMTN-00690 | SAV0573 | proP | 649645 | 651045 | 650392 | | |
| NMTN-03590 | SAV0573 | proP | 649645 | 651045 | 650674 | | |
| NMTN-03745 | SAV0573 | proP | 649645 | 651045 | 650753 | | |
| NMTN-14607 | SAV0573 | proP | 649645 | 651045 | 650923 | | |
| NMTN-08116 | SAV0573 | proP | 649645 | 651045 | 651007 | | |
| NMTN-08305 | SAV0574 | | 651380 | 651589 | 651453 | + | hypothetical protein |
| NMTN-09443 | SAV0574 | | 651380 | 651589 | 651557 | | |
| NMTN-08717 | SAV0575 | vraA | 651589 | 652965 | 651848 | + | hypothetical protein |
| NMTN-06125 | SAV0575 | vraA | 651589 | 652965 | 652331 | | |
| NMTN-02468 | SAV0575 | vraA | 651589 | 652965 | 652516 | | |
| NMTN-04497 | SAV0575 | vraA | 651589 | 652965 | 652526 | | |
| NMTN-13328 | SAV0576 | vraB | 652967 | 654106 | 653076 | + | acetyl-CoA c-acetyltransferase |
| NMTN-12947 | SAV0576 | vraB | 652967 | 654106 | 653287 | | |
| NMTN-05028 | SAV0576 | vraB | 652967 | 654106 | 653952 | | |
| NMTN-06188 | SAV0577 | vraC | 654081 | 654446 | 654314 | + | hypothetical protein |
| NMTN-04977 | SAV0580 | | 655593 | 656423 | 656185 | | hypothetical protein |
| NMTN-03482 | SAV0580 | | 655593 | 656423 | 656284 | | |
| NMTN-14469 | SAV0581 | ung | 656607 | 657263 | 656836 | + | uracil-DNA glycosylase |
| NMTN-10785 | SAV0581 | ung | 656607 | 657263 | 656870 | | |
| NMTN-06170 | SAV0583 | | 657777 | 658145 | 657942 | + | conserved hypothetical protein |
| NMTN-14460 | SAV0584 | | 658266 | 659750 | 658269 | + | hypothetical protein |
| NMTN-12340 | SAV0584 | | 658266 | 659750 | 659307 | | |
| NMTN-05277 | SAV0584 | | 658266 | 659750 | 659385 | | |
| NMTN-09817 | SAV0584 | | 658266 | 659750 | 659691 | | |
| NMTN-02439 | SAV0586 | | 660356 | 661006 | 660655 | − | conserved hypothetical protein |
| NMTN-01545 | SAV0588 | pta | 662589 | 663575 | 663052 | + | phosphotransacetylase |
| NMTN-07133 | SAV0594 | | 668568 | 670016 | 668643 | − | mercuric reductase homologue |
| NMTN-03429 | SAV0594 | | 668568 | 670016 | 668708 | | |
| NMTN-09528 | SAV0594 | | 668568 | 670016 | 668795 | | |
| NMTN-13336 | SAV0594 | | 668568 | 670016 | 668988 | | |
| NMTN-04683 | SAV0594 | | 668568 | 670016 | 669190 | | |
| NMTN-06257 | SAV0594 | | 668568 | 670016 | 669580 | | |
| NMTN-04801 | SAV0594 | | 668568 | 670016 | 669635 | | |
| NMTN-01995 | SAV0594 | | 668568 | 670016 | 669728 | | |
| NMTN-10610 | SAV0594 | | 668568 | 670016 | 669752 | | |
| NMTN-13692 | SAV0594 | | 668568 | 670016 | 669881 | | |
| NMTN-13692 | SAV0595 | | 669877 | 670317 | 669881 | − | hypothetical protein |
| NMTN-10439 | SAV0595 | | 669877 | 670317 | 670248 | | |
| NMTN-09988 | SAV0596 | | 670942 | 672336 | 671033 | + | conserved hypothetical protein |
| NMTN-09234 | SAV0596 | | 670942 | 672336 | 671218 | | |
| NMTN-12757 | SAV0596 | | 670942 | 672336 | 671365 | | |
| NMTN-14716 | SAV0596 | | 670942 | 672336 | 671713 | | |
| NMTN-04142 | SAV0596 | | 670942 | 672336 | 671746 | | |
| NMTN-13135 | SAV0596 | | 670942 | 672336 | 671965 | | |
| NMTN-05481 | SAV0596 | | 670942 | 672336 | 672218 | | |
| NMTN-05200 | SAV0597 | | 672340 | 672972 | 672356 | + | conserved hypothetical protein |
| NMTN-07877 | SAV0597 | | 672340 | 672972 | 672481 | | |
| NMTN-14639 | SAV0597 | | 672340 | 672972 | 672692 | | |
| NMTN-02981 | SAV0598 | | 673019 | 673639 | 673203 | | |
| NMTN-06547 | SAV0598 | | 673019 | 673639 | 673237 | + | conserved hypothetical protein |
| NMTN-09457 | SAV0598 | | 673019 | 673639 | 673399 | | |
| NMTN-11522 | SAV0598 | | 673019 | 673639 | 673507 | | |
| NMTN-13785 | SAV0598 | | 673019 | 673639 | 673607 | | |
| NMTN-08656 | SAV0600 | | 674859 | 675797 | 674881 | + | hypothetical protein |
| NMTN-02070 | SAV0600 | | 674859 | 675797 | 674931 | | |
| NMTN-03972 | SAV0600 | | 674859 | 675797 | 675227 | | |
| NMTN-00119 | SAV0600 | | 674859 | 675797 | 675230 | | |
| NMTN-04979 | SAV0600 | | 674859 | 675797 | 675399 | | |
| NMTN-04560 | SAV0600 | | 674859 | 675797 | 675606 | | |
| NMTN-14280 | SAV0601 | | 676243 | 676779 | 676380 | + | conserved hypothetical protein |
| NMTN-05263 | SAV0601 | | 676243 | 676779 | 676678 | | |
| NMTN-12002 | SAV0602 | | 676947 | 677423 | 677288 | + | conserved hypothetical protein |
| NMTN-01599 | SAV0603 | | 677463 | 678758 | 677478 | + | conserved hypothetical protein |
| NMTN-11312 | SAV0603 | | 677463 | 678758 | 677481 | | |
| NMTN-12108 | SAV0603 | | 677463 | 678758 | 677932 | | |
| NMTN-04476 | SAV0603 | | 677463 | 678758 | 678032 | | |
| NMTN-13423 | SAV0603 | | 677463 | 678758 | 678340 | | |
| NMTN-11487 | SAV0603 | | 677463 | 678758 | 678727 | | |
| NMTN-05238 | SAV0604 | | 678801 | 679307 | 679228 | + | conserved hypothetical protein |
| NMTN-04470 | SAV0605 | adh1 | 679806 | 680816 | 680646 | + | alcohol dehydrogenase I |
| NMTN-09946 | SAV0605 | adh1 | 679806 | 680816 | 680730 | | |
| NMTN-08866 | SAV0605 | adh1 | 679806 | 680816 | 680787 | | |
| NMTN-06320 | SAV0609 | | 684323 | 685210 | 684729 | + | hypothetical protein |
| NMTN-00139 | SAV0609 | | 684323 | 685210 | 684897 | | |
| NMTN-06300 | SAV0609 | | 684323 | 685210 | 684938 | | |
| NMTN-12062d | SAV0609 | | 684323 | 685210 | 684996 | | |
| NMTN-06584 | SAV0609 | | 684323 | 685210 | 685079 | | |
| NMTN-04481 | SAV0610 | | 685359 | 686309 | 685414 | + | hypothetical protein |
| NMTN-07453 | SAV0610 | | 685359 | 686309 | 685562 | | |
| NMTN-05594 | SAV0610 | | 685359 | 686309 | 685888 | | |
| NMTN-06185 | SAV0610 | | 685359 | 686309 | 685929 | | |
| NMTN-13687 | SAV0611 | | 686364 | 687083 | 686415 | + | hypothetical protein |
| NMTN-01637 | SAV0611 | | 686364 | 687083 | 686459 | | |
| NMTN-13151 | SAV0611 | | 686364 | 687083 | 686645 | | |
| NMTN-09856 | SAV0611 | | 686364 | 687083 | 686796 | | |
| NMTN-12058 | SAV0611 | | 686364 | 687083 | 686842 | | |
| NMTN-14497 | SAV0611 | | 686364 | 687083 | 686878 | | |
| NMTN-00604 | SAV0611 | | 686364 | 687083 | 686881 | | |
| NMTN-03143 | SAV0612 | | 687067 | 687867 | 687379 | + | hypothetical protein |
| NMTN-08159 | SAV0612 | | 687067 | 687867 | 687406 | | |
| NMTN-07275 | SAV0614 | | 688671 | 689387 | 689387 | + | hypothetical protein |
| NMTN-06666 | SAV0615 | | 689556 | 690344 | 689628 | + | hypothetical protein |
| NMTN-09461 | SAV0615 | | 689556 | 690344 | 689826 | | |
| NMTN-09652 | SAV0615 | | 689556 | 690344 | 690125 | | |
| NMTN-03663 | SAV0615 | | 689556 | 690344 | 690136 | | |
| NMTN-14744 | SAV0615 | | 689556 | 690344 | 690344 | | |
| NMTN-10017 | SAV0616 | sarA | 690598 | 690972 | 690866 | − | staphylococcal accessory regulator A |
| NMTN-13209 | SAV0617 | | 691920 | 692849 | 692308 | − | conserved hypothetical protein |
| NMTN-00330 | SAV0617 | | 691920 | 692849 | 692323 | | |
| NMTN-03709 | SAV0617 | | 691920 | 692849 | 692549 | | |
| NMTN-05556 | SAV0617 | | 691920 | 692849 | 692627 | | |
| NMTN-06515 | SAV0617 | | 691920 | 692849 | 692676 | | |
| NMTN-09404 | SAV0618 | | 693044 | 693268 | 693100 | − | hypothetical protein |
| NMTN-03162 | SAV0620 | | 693647 | 694207 | 693659 | + | hypothetical protein |
| NMTN-04096 | SAV0620 | | 693647 | 694207 | 693899 | | |
| NMTN-13634 | SAV0620 | | 693647 | 694207 | 694031 | | |
| NMTN-11927 | SAV0620 | | 693647 | 694207 | 694120 | | |
| NMTN-11296 | SAV0620 | | 693647 | 694207 | 694179 | | |
| NMTN-11754 | SAV0621 | | 694226 | 696250 | 694738 | + | hypothetical protein |
| NMTN-08881 | SAV0621 | | 694226 | 696250 | 695280 | | |
| NMTN-10452 | SAV0621 | | 694226 | 696250 | 695443 | | |
| NMTN-04837 | SAV0621 | | 694226 | 696250 | 695569 | | |
| NMTN-12298 | SAV0621 | | 694226 | 696250 | 695861 | | |
| NMTN-11326 | SAV0622 | | 696296 | 696628 | 696440 | + | hypothetical protein |
| NMTN-00286 | SAV0625 | | 697371 | 698867 | 697434 | + | MnhD homologue |
| NMTN-11745 | SAV0625 | | 697371 | 698867 | 697456 | | |
| NMTN-02419 | SAV0625 | | 697371 | 698867 | 697809 | | |
| NMTN-01806 | SAV0625 | | 697371 | 698867 | 698129 | | |
| NMTN-00821 | SAV0625 | | 697371 | 698867 | 698412 | | |
| NMTN-14384 | SAV0626 | | 698868 | 699350 | 699265 | + | hypothetical protein |
| NMTN-15297 | SAV0626 | | 698868 | 699350 | 699289 | | |
| NMTN-13291 | SAV0628 | | 699624 | 700061 | 699976 | + | conserved hypothetical protein |
| NMTN-13474 | SAV0629 | | 700401 | 702443 | 700407 | + | conserved hypothetical protein |
| NMTN-01786 | SAV0629 | | 700401 | 702443 | 701154 | | |
| NMTN-14555 | SAV0629 | | 700401 | 702443 | 701229 | | |
| NMTN-15296 | SAV0629 | | 700401 | 702443 | 701648 | | |
| NMTN-02474 | SAV0629 | | 700401 | 702443 | 701742 | | |
| NMTN-05555 | SAV0629 | | 700401 | 702443 | 701742 | | |
| NMTN-05588 | SAV0629 | | 700401 | 702443 | 701755 | | |
| NMTN-06410 | SAV0629 | | 700401 | 702443 | 701967 | | |
| NMTN-01956 | SAV0629 | | 700401 | 702443 | 702003 | | |
| NMTN-13725 | SAV0629 | | 700401 | 702443 | 702004 | | |
| NMTN-12361 | SAV0629 | | 700401 | 702443 | 702006 | | |
| NMTN-08490 | SAV0629 | | 700401 | 702443 | 702012 | | |
| NMTN-10554 | SAV0629 | | 700401 | 702443 | 702032 | | |
| NMTN-13312 | SAV0629 | | 700401 | 702443 | 702038 | | |
| NMTN-01485 | SAV0631 | | 704990 | 705919 | 705166 | − | lipoprotein |
| NMTN-08869 | SAV0631 | | 704990 | 705919 | 705901 | | |
| NMTN-14394 | SAV0632 | | 705916 | 706752 | 706277 | − | conserved hypothetical protein |
| NMTN-09063 | SAV0632 | | 705916 | 706752 | 706599 | | |
| NMTN-02717 | SAV0632 | | 705916 | 706752 | 706658 | | |
| NMTN-08894 | SAV0633 | | 706746 | 707489 | 707275 | − | hypothetical protein |
| NMTN-09193 | SAV0633 | | 706746 | 707489 | 707452 | | |
| NMTN-04913 | SAV0635 | | 708335 | 709087 | 708946 | − | hypothetical protein |
| NMTN-14463 | SAV0635 | | 708335 | 709087 | 708947 | | |
| NMTN-12156 | SAV0635 | | 708335 | 709087 | 709040 | | |
| NMTN-08883 | SAV0640 | tagX | 713354 | 714361 | 713968 | + | teichoic acid biosynthesis protein X |
| NMTN-05382 | SAV0640 | tagX | 713354 | 714361 | 714040 | | |
| NMTN-11552 | SAV0642 | pbp4 | 714939 | 716234 | 715333 | − | penicillin binding protein 4 |
| NMTN-13662 | SAV0643 | | 716655 | 718382 | 717066 | + | ATP-binding cassette transporter A |
| NMTN-02038 | SAV0643 | | 716655 | 718382 | 717187 | | |
| NMTN-09403 | SAV0643 | | 716655 | 718382 | 717645 | | |
| NMTN-11569 | SAV0643 | | 716655 | 718382 | 717805 | | |
| NMTN-12631 | SAV0643 | | 716655 | 718382 | 717868 | | |
| NMTN-05177 | SAV0643 | | 716655 | 718382 | 717931 | | |
| NMTN-14458 | SAV0643 | | 716655 | 718382 | 718050 | | |
| NMTN-12855 | SAV0644 | | 718451 | 718531 | 718460 | + | hypothetical protein |
| NMTN-09195 | SAV0644 | | 718451 | 718531 | 718521 | | |
| NMTN-03704 | SAV0645 | | 718739 | 719968 | 719062 | + | hypothetical protein |
| NMTN-10995 | SAV0645 | | 718739 | 719968 | 719111 | | |
| NMTN-13702 | SAV0645 | | 718739 | 719968 | 719533 | | |
| NMTN-02716 | SAV0645 | | 718739 | 719968 | 719553 | | |
| NMTN-03100 | SAV0646 | | 720715 | 721326 | 720713 | + | conserved hypothetical protein |
| NMTN-11205 | SAV0647 | fhuA | 721608 | 722405 | 722118 | + | ferrichrome transport ATP-binding |
| | | | | | | | protein |
| NMTN-08160 | SAV0647 | fhuA | 721608 | 722405 | 722302 | | |
| NMTN-08211 | SAV0647 | fhuA | 721608 | 722405 | 722364 | | |
| NMTN-03749 | SAV0648 | fhuB | 722441 | 723445 | 722573 | + | ferrichrome transport permease |
| NMTN-12142 | SAV0648 | fhuB | 722441 | 723445 | 722663 | | |
| NMTN-07433d | SAV0648 | fhuB | 722441 | 723445 | 722877 | | |
| NMTN-07881 | SAV0648 | fhuB | 722441 | 723445 | 722942 | | |
| NMTN-07882 | SAV0648 | fhuB | 722441 | 723445 | 722968 | | |
| NMTN-02314 | SAV0648 | fhuB | 722441 | 723445 | 723344 | | |
| NMTN-02667 | SAV0649 | fhuG | 723442 | 724458 | 723550 | + | ferrichrome transport permease |
| NMTN-03223 | SAV0649 | fhuG | 723442 | 724458 | 723725 | | |
| NMTN-05008 | SAV0649 | fhuG | 723442 | 724458 | 723808 | | |
| NMTN-00285 | SAV0649 | fhuG | 723442 | 724458 | 723959 | | |
| NMTN-12124 | SAV0649 | fhuG | 723442 | 724458 | 723959 | | |
| NMTN-06179 | SAV0649 | fhuG | 723442 | 724458 | 724133 | | |
| NMTN-12862d | SAV0650 | | 724695 | 725660 | 725492 | + | hypothetical protein |
| NMTN-09237 | SAV0650 | | 724695 | 725660 | 725536 | | |
| NMTN-08830 | SAV0652 | | 726279 | 726641 | 726364 | + | conserved hypothetical protein |
| NMTN-06391 | SAV0652 | | 726279 | 726641 | 726387 | | |
| NMTN-02257 | SAV0652 | | 726279 | 726641 | 726579 | | |
| NMTN-02489 | SAV0653 | | 726757 | 727254 | 726784 | + | hypothetical protein |
| NMTN-02384 | SAV0653 | | 726757 | 727254 | 726918 | | |
| NMTN-01585 | SAV0653 | | 726757 | 727254 | 727109 | | |
| NMTN-09478 | SAV0654 | | 727798 | 728778 | 728068 | + | conserved hypothetical protein |
| NMTN-02849 | SAV0654 | | 727798 | 728778 | 728131 | | |
| NMTN-12623 | SAV0654 | | 727798 | 728778 | 728170 | | |
| NMTN-12611 | SAV0654 | | 727798 | 728778 | 728436 | | |
| NMTN-06198 | SAV0654 | | 727798 | 728778 | 728513 | | |
| NMTN-09147 | SAV0654 | | 727798 | 728778 | 728621 | | |
| NMTN-12362 | SAV0654 | | 727798 | 728778 | 728668 | | |
| NMTN-02091 | SAV0655 | | 728929 | 729972 | 728956 | + | hypothetical protein |
| NMTN-09658 | SAV0655 | | 728929 | 729972 | 729124 | | |
| NMTN-03120 | SAV0655 | | 728929 | 729972 | 729808 | | |
| NMTN-04905 | SAV0655 | | 728929 | 729972 | 729808 | | |
| NMTN-06583 | SAV0656 | | 730301 | 730729 | 730477 | + | hypothetical protein |
| NMTN-14397 | SAV0656 | | 730301 | 730729 | 730622 | | |
| NMTN-00623 | SAV0656 | | 730301 | 730729 | 730691 | | |
| NMTN-11443 | SAV0656 | | 730301 | 730729 | 730691 | | |
| NMTN-11410 | SAV0657 | | 730938 | 731444 | 731218 | − | conserved hypothetical protein |
| NMTN-03803 | SAV0657 | | 730938 | 731444 | 731422 | | |
| NMTN-09891 | SAV0658 | | 731557 | 732480 | 731574 | + | hypothetical protein |
| NMTN-11269 | SAV0658 | | 731557 | 732480 | 731597 | | |
| NMTN-03813 | SAV0658 | | 731557 | 732480 | 731604 | | |
| NMTN-03115 | SAV0658 | | 731557 | 732480 | 731940 | | |
| NMTN-08281 | SAV0658 | | 731557 | 732480 | 731995 | | |
| NMTN-05150 | SAV0658 | | 731557 | 732480 | 732040 | | |
| NMTN-02700 | SAV0658 | | 731557 | 732480 | 732154 | | |
| NMTN-11930 | SAV0658 | | 731557 | 732480 | 732159 | | |
| NMTN-13761d | SAV0658 | | 731557 | 732480 | 732161 | | |
| NMTN-04324 | SAV0659 | | 732496 | 733170 | 732608 | + | hypothetical protein |
| NMTN-07802 | SAV0659 | | 732496 | 733170 | 732883 | | |
| NMTN-12529 | SAV0659 | | 732496 | 733170 | 733118 | | |
| NMTN-11539 | SAV0660 | | 733163 | 734203 | 733446 | + | hypothetical protein |
| NMTN-13161 | SAV0660 | | 733163 | 734203 | 733630 | | |
| NMTN-03902 | SAV0660 | | 733163 | 734203 | 733669 | | |
| NMTN-03996 | SAV0660 | | 733163 | 734203 | 733677 | | |
| NMTN-05171 | SAV0660 | | 733163 | 734203 | 733816 | | |
| NMTN-08661 | SAV0660 | | 733163 | 734203 | 733971 | | |
| NMTN-12657 | SAV0661 | vraF | 734347 | 735108 | 734497 | + | ABC transporter ATP-binding protein |
| NMTN-12743 | SAV0661 | vraF | 734347 | 735108 | 734756 | | |
| NMTN-00587 | SAV0661 | vraF | 734347 | 735108 | 734822 | | |
| NMTN-12065 | SAV0662 | vraG | 735086 | 736987 | 735287 | + | ABC transporter permease |
| NMTN-04764 | SAV0662 | vraG | 735086 | 736987 | 735383 | | |
| NMTN-10635 | SAV0662 | vraG | 735086 | 736987 | 735407 | | |
| NMTN-10002 | SAV0662 | vraG | 735086 | 736987 | 735614 | | |
| NMTN-12433 | SAV0662 | vraG | 735086 | 736987 | 735716 | | |
| NMTN-04345 | SAV0662 | vraG | 735086 | 736987 | 735748 | | |
| NMTN-11491 | SAV0662 | vraG | 735086 | 736987 | 735893 | | |
| NMTN-14400 | SAV0662 | vraG | 735086 | 736987 | 736002 | | |
| NMTN-12418 | SAV0662 | vraG | 735086 | 736987 | 736179 | | |
| NMTN-08348 | SAV0662 | vraG | 735086 | 736987 | 736224 | | |
| NMTN-12913 | SAV0662 | vraG | 735086 | 736987 | 736241 | | |
| NMTN-07575 | SAV0662 | vraG | 735086 | 736987 | 736402 | | |
| NMTN-13760 | SAV0662 | vraG | 735086 | 736987 | 736569 | | |
| NMTN-13738 | SAV0662 | vraG | 735086 | 736987 | 736781 | | |
| NMTN-07723 | SAV0662 | vraG | 735086 | 736987 | 736833 | | |
| NMTN-03532 | SAV0662 | vraG | 735086 | 736987 | 736927 | | |
| NMTN-03538 | SAV0662 | vraG | 735086 | 736987 | 736927 | | |
| NMTN-03539 | SAV0662 | vraG | 735086 | 736987 | 736927 | | |
| NMTN-11182 | SAV0662 | vraG | 735086 | 736987 | 736948 | | |
| NMTN-11188 | SAV0662 | vraG | 735086 | 736987 | 736948 | | |
| NMTN-09610 | SAV0663 | | 737710 | 738327 | 737791 | + | conserved hypothetical protein |
| NMTN-05491 | SAV0663 | | 737710 | 738327 | 738009 | | |
| NMTN-03029 | SAV0664 | | 738343 | 739350 | 738351 | + | hypothetical protein |
| NMTN-11854 | SAV0664 | | 738343 | 739350 | 739012 | | |
| NMTN-12204 | SAV0665 | | 739938 | 740735 | 739954 | − | secretory antigen SsaA homologue |
| NMTN-09455 | SAV0665 | | 739938 | 740735 | 740035 | | |
| NMTN-06444 | SAV0667 | | 742437 | 744587 | 742740 | + | hypothetical protein |
| NMTN-11776 | SAV0667 | | 742437 | 744587 | 743087 | | |
| NMTN-13409 | SAV0667 | | 742437 | 744587 | 743089 | | |
| NMTN-14641 | SAV0667 | | 742437 | 744587 | 743100 | | |
| NMTN-11555 | SAV0667 | | 742437 | 744587 | 743304 | | |
| NMTN-05202 | SAV0667 | | 742437 | 744587 | 743316 | | |
| NMTN-04824 | SAV0667 | | 742437 | 744587 | 744019 | | |
| NMTN-09757 | SAV0667 | | 742437 | 744587 | 744277 | | |
| NMTN-09616 | SAV0667 | | 742437 | 744587 | 744377 | | |
| NMTN-12771 | SAV0668 | | 744667 | 745092 | 745040 | + | hypothetical protein |
| NMTN-04018 | SAV0669 | | 745558 | 745998 | 745963 | + | conserved hypothetical protein |
| NMTN-03602 | SAV0672 | | 747770 | 748450 | 748055 | + | hypothetical protein |
| NMTN-12715 | SAV0672 | | 747770 | 748450 | 748220 | | |
| NMTN-02263 | SAV0672 | | 747770 | 748450 | 748256 | | |
| NMTN-08050 | SAV0673 | | 748588 | 749802 | 749130 | + | hypothetical protein |
| NMTN-06167 | SAV0673 | | 748588 | 749802 | 749292 | | |
| NMTN-14710 | SAV0673 | | 748588 | 749802 | 749380 | | |
| NMTN-11906 | SAV0673 | | 748588 | 749802 | 749509 | | |
| NMTN-01948 | SAV0673 | | 748588 | 749802 | 749567 | | |
| NMTN-11566 | SAV0673 | | 748588 | 749802 | 749574 | | |
| NMTN-03271 | SAV0673 | | 748588 | 749802 | 749752 | | |
| NMTN-01974 | SAV0674 | | 749799 | 750287 | 749889 | + | conserved hypothetical protein |
| NMTN-06847 | SAV0674 | | 749799 | 750287 | 750007 | | |
| NMTN-10215 | SAV0675 | | 750386 | 751075 | 750409 | − | conserved hypothetical protein |
| NMTN-05114 | SAV0675 | | 750386 | 751075 | 750649 | | |
| NMTN-05583 | SAV0675 | | 750386 | 751075 | 750822 | | |
| NMTN-07807 | SAV0676 | | 751202 | 751645 | 751203 | + | hypothetical protein |
| NMTN-00507 | SAV0676 | | 751202 | 751645 | 751583 | | |
| NMTN-01633 | SAV0676 | | 751202 | 751645 | 751642 | | |
| NMTN-07528 | SAV0679 | | 752624 | 753166 | 752654 | + | conserved hypothetical protein |
| NMTN-09638 | SAV0679 | | 752624 | 753166 | 752696 | | |
| NMTN-09740 | SAV0679 | | 752624 | 753166 | 752696 | | |
| NMTN-08055 | SAV0679 | | 752624 | 753166 | 752715 | | |
| NMTN-08045 | SAV0679 | | 752624 | 753166 | 752717 | | |
| NMTN-10406 | SAV0679 | | 752624 | 753166 | 752729 | | |
| NMTN-00785 | SAV0679 | | 752624 | 753166 | 752775 | | |
| NMTN-14448 | SAV0679 | | 752624 | 753166 | 752855 | | |
| NMTN-06329 | SAV0679 | | 752624 | 753166 | 752892 | | |
| NMTN-02793 | SAV0679 | | 752624 | 753166 | 752918 | | |
| NMTN-10019 | SAV0679 | | 752624 | 753166 | 753095 | | |
| NMTN-02754 | SAV0679 | | 752624 | 753166 | 753107 | | |
| NMTN-10083 | SAV0679 | | 752624 | 753166 | 753154 | | |
| NMTN-09951 | SAV0680 | | 753262 | 753828 | 753443 | − | conserved hypothetical protein |
| NMTN-05086 | SAV0680 | | 753262 | 753828 | 753778 | | |
| NMTN-06973 | SAV0681 | | 753830 | 754288 | 754079 | − | conserved hypothetical protein |
| NMTN-01789 | SAV0681 | | 753830 | 754288 | 754139 | | |
| NMTN-00836 | SAV0681 | | 753830 | 754288 | 754150 | | |
| NMTN-08015 | SAV0682 | | 754291 | 754974 | 754518 | − | conserved hypothetical protein |
| NMTN-06681 | SAV0684 | | 756240 | 757871 | 756423 | + | hypothetical protein |
| NMTN-00316 | SAV0684 | | 756240 | 757871 | 756948 | | |
| NMTN-04157 | SAV0684 | | 756240 | 757871 | 757181 | | |
| NMTN-07614 | SAV0684 | | 756240 | 757871 | 757383 | | |
| NMTN-00617 | SAV0685 | | 757868 | 759541 | 757916 | + | hypothetical protein |
| NMTN-12930 | SAV0685 | | 757868 | 759541 | 758054 | | |
| NMTN-02741 | SAV0685 | | 757868 | 759541 | 758246 | | |
| NMTN-05141 | SAV0685 | | 757868 | 759541 | 758289 | | |
| NMTN-12352 | SAV0685 | | 757868 | 759541 | 758702 | | |
| NMTN-13905 | SAV0685 | | 757868 | 759541 | 758839 | | |
| NMTN-03671 | SAV0685 | | 757868 | 759541 | 759021 | | |
| NMTN-14881 | SAV0685 | | 757868 | 759541 | 759037 | | |
| NMTN-14437 | SAV0685 | | 757868 | 759541 | 759048 | | |
| NMTN-14454 | SAV0685 | | 757868 | 759541 | 759048 | | |
| NMTN-14461 | SAV0685 | | 757868 | 759541 | 759048 | | |
| NMTN-14474 | SAV0685 | | 757868 | 759541 | 759048 | | |
| NMTN-14540 | SAV0685 | | 757868 | 759541 | 759048 | | |
| NMTN-14543 | SAV0685 | | 757868 | 759541 | 759048 | | |
| NMTN-14545 | SAV0685 | | 757868 | 759541 | 759048 | | |
| NMTN-14546 | SAV0685 | | 757868 | 759541 | 759048 | | |
| NMTN-14548 | SAV0685 | | 757868 | 759541 | 759048 | | |
| NMTN-14550 | SAV0685 | | 757868 | 759541 | 759048 | | |
| NMTN-14791 | SAV0685 | | 757868 | 759541 | 759048 | | |
| NMTN-14856 | SAV0685 | | 757868 | 759541 | 759048 | | |
| NMTN-13763 | SAV0685 | | 757868 | 759541 | 759123 | | |
| NMTN-00577 | SAV0685 | | 757868 | 759541 | 759287 | | |
| NMTN-10266 | SAV0685 | | 757868 | 759541 | 759304 | | |
| NMTN-14538 | SAV0685 | | 757868 | 759541 | 759394 | | |
| NMTN-09679 | SAV0685 | | 757868 | 759541 | 759515 | | |
| NMTN-03040 | SAV0686 | | 759668 | 760111 | 760078 | − | hypothetical protein |
| NMTN-11829 | SAV0687 | | 760338 | 761264 | 760424 | + | hypothetical protein |
| NMTN-04065 | SAV0687 | | 760338 | 761264 | 760875 | | |
| NMTN-07605 | SAV0687 | | 760338 | 761264 | 760903 | | |
| NMTN-07605 | SAV0687 | | 760338 | 761264 | 760903 | | |
| NMTN-03974 | SAV0688 | | 761367 | 762275 | 761367 | + | hypothetical protein |
| NMTN-02580 | SAV0688 | | 761367 | 762275 | 761465 | | |
| NMTN-10034 | SAV0688 | | 761367 | 762275 | 761738 | | |
| NMTN-14621 | SAV0688 | | 761367 | 762275 | 761797 | | |
| NMTN-08993 | SAV0688 | | 761367 | 762275 | 761902 | | |
| NMTN-09003 | SAV0688 | | 761367 | 762275 | 761902 | | |
| NMTN-10891 | SAV0688 | | 761367 | 762275 | 762177 | | |
| NMTN-06398 | SAV0688 | | 761367 | 762275 | 762277 | | |
| NMTN-10986 | SAV0690 | | 762880 | 764433 | 763171 | + | conserved hypothetical protein |
| NMTN-13506 | SAV0690 | | 762880 | 764433 | 763538 | | |
| NMTN-12280 | SAV0691 | | 764519 | 765892 | 764941 | + | hypothetical protein |
| NMTN-14693 | SAV0691 | | 764519 | 765892 | 765366 | | |
| NMTN-11360 | SAV0692 | | 766047 | 766331 | 766307 | − | hypothetical protein |
| NMTN-02306 | SAV0693 | | 766328 | 766948 | 766389 | − | conserved hypothetical protein |
| NMTN-04797 | SAV0693 | | 766328 | 766948 | 766646 | | |
| NMTN-12979d | SAV0693 | | 766328 | 766948 | 766758 | | |
| NMTN-11397 | SAV0693 | | 766328 | 766948 | 766761 | | |
| NMTN-08465 | SAV0693 | | 766328 | 766948 | 766762 | | |
| NMTN-07256 | SAV0693 | | 766328 | 766948 | 766834 | | |
| NMTN-09710 | SAV0694 | | 767131 | 767553 | 767397 | − | conserved hypothetical protein |
| NMTN-05267 | SAV0695 | norA | 767762 | 768928 | 768480 | + | quinolone resistance protein |
| NMTN-14499 | SAV0695 | norA | 767762 | 768928 | 768489 | | |
| NMTN-11290 | SAV0695 | norA | 767762 | 768928 | 768509 | | |
| NMTN-06619 | SAV0695 | norA | 767762 | 768928 | 768529 | | |
| NMTN-09594 | SAV0696 | | 769221 | 769673 | 769538 | + | hypothetical protein |
| NMTN-02147 | SAV0696 | | 769221 | 769673 | 769552 | | |
| NMTN-11344 | SAV0696 | | 769221 | 769673 | 769594 | | |
| NMTN-04834 | SAV0696 | | 769221 | 769673 | 769598 | | |
| NMTN-13306 | SAV0696 | | 769221 | 769673 | 769600 | | |
| NMTN-04999 | SAV0696 | | 769221 | 769673 | 769604 | | |
| NMTN-09918 | SAV0698 | | 770582 | 771343 | 770646 | + | hypothetical protein |
| NMTN-13698 | SAV0699 | fruB | 771340 | 772260 | 771544 | + | fructose 1-phosphate kinase |
| NMTN-09835 | SAV0699 | fruB | 771340 | 772260 | 771605 | | |
| NMTN-15260d | SAV0700 | fruA | 772266 | 774224 | 772790 | + | fructose specific permease |
| NMTN-07862 | SAV0700 | fruA | 772266 | 774224 | 773210 | | |
| NMTN-07866 | SAV0700 | fruA | 772266 | 774224 | 773210 | | |
| NMTN-09792 | SAV0700 | fruA | 772266 | 774224 | 773664 | | |
| NMTN-04930 | SAV0700 | fruA | 772266 | 774224 | 773713 | | |
| NMTN-13355 | SAV0700 | fruA | 772266 | 774224 | 773921 | | |
| NMTN-01714 | SAV0700 | fruA | 772266 | 774224 | 774132 | | |
| NMTN-00659 | SAV0701 | nagA | 774532 | 775713 | 774828 | + | probable N-acetylglucosamine-6- |
| | | | | | | | phosphate deacetylase |
| NMTN-10016 | SAV0701 | nagA | 774532 | 775713 | 775011 | | |
| NMTN-14408 | SAV0701 | nagA | 774532 | 775713 | 775161 | | |
| NMTN-09968 | SAV0701 | nagA | 774532 | 775713 | 775362 | | |
| NMTN-13343 | SAV0702 | | 775934 | 777283 | 775971 | + | hypothetical protein |
| NMTN-05064 | SAV0702 | | 775934 | 777283 | 776087 | | |
| NMTN-06952 | SAV0702 | | 775934 | 777283 | 776254 | | |
| NMTN-06436 | SAV0702 | | 775934 | 777283 | 776898 | | |
| NMTN-09643 | SAV0702 | | 775934 | 777283 | 777005 | | |
| NMTN-09837 | SAV0703 | | 777505 | 778344 | 777578 | + | hypothetical protein |
| NMTN-10218 | SAV0703 | | 777505 | 778344 | 777682 | | |
| NMTN-10069 | SAV0703 | | 777505 | 778344 | 777851 | | |
| NMTN-10602 | SAV0703 | | 777505 | 778344 | 778233 | | |
| NMTN-04645 | SAV0704 | | 778476 | 779459 | 778605 | + | hypothetical protein |
| NMTN-12955 | SAV0704 | | 778476 | 779459 | 778711 | | |
| NMTN-11490 | SAV0704 | | 778476 | 779459 | 778855 | | |
| NMTN-09783 | SAV0704 | | 778476 | 779459 | 779125 | | |
| NMTN-10443 | SAV0704 | | 778476 | 779459 | 779125 | | |
| NMTN-07659 | SAV0704 | | 778476 | 779459 | 779195 | | |
| NMTN-09808 | SAV0704 | | 778476 | 779459 | 779204 | | |
| NMTN-07936 | SAV0704 | | 778476 | 779459 | 779344 | | |
| NMTN-08978 | SAV0704 | | 778476 | 779459 | 779345 | | |
| NMTN-12334 | SAV0705 | saeS | 779531 | 780586 | 779584 | − | histidine protein kinase |
| NMTN-06298 | SAV0705 | saeS | 779531 | 780586 | 779587 | | |
| NMTN-08696 | SAV0705 | saeS | 779531 | 780586 | 779596 | | |
| NMTN-09887 | SAV0705 | saeS | 779531 | 780586 | 779645 | | |
| NMTN-09810 | SAV0705 | saeS | 779531 | 780586 | 779674 | | |
| NMTN-09885 | SAV0705 | saeS | 779531 | 780586 | 779679 | | |
| NMTN-08778 | SAV0705 | saeS | 779531 | 780586 | 779712 | | |
| NMTN-09752 | SAV0705 | saeS | 779531 | 780586 | 779723 | | |
| NMTN-09755 | SAV0705 | saeS | 779531 | 780586 | 779755 | | |
| NMTN-05467 | SAV0705 | saeS | 779531 | 780586 | 779799 | | |
| NMTN-11987 | SAV0705 | saeS | 779531 | 780586 | 779799 | | |
| NMTN-09734 | SAV0705 | saeS | 779531 | 780586 | 779923 | | |
| NMTN-07762 | SAV0705 | saeS | 779531 | 780586 | 779957 | | |
| NMTN-09754 | SAV0705 | saeS | 779531 | 780586 | 779979 | | |
| NMTN-13260 | SAV0705 | saeS | 779531 | 780586 | 780091 | | |
| NMTN-07904 | SAV0705 | saeS | 779531 | 780586 | 780162 | | |
| NMTN-12384 | SAV0705 | saeS | 779531 | 780586 | 780375 | | |
| NMTN-09725 | SAV0705 | saeS | 779531 | 780586 | 780399 | | |
| NMTN-11179 | SAV0705 | saeS | 779531 | 780586 | 780421 | | |
| NMTN-02321 | SAV0705 | saeS | 779531 | 780586 | 780445 | | |
| NMTN-10567 | SAV0705 | saeS | 779531 | 780586 | 780504 | | |
| NMTN-08853 | SAV0705 | saeS | 779531 | 780586 | 780519 | | |
| NMTN-12661 | SAV0705 | saeS | 779531 | 780586 | 780540 | | |
| NMTN-12747 | SAV0706 | saeR | 780586 | 781272 | 780605 | − | response regulator |
| NMTN-08517 | SAV0706 | saeR | 780586 | 781272 | 780627 | | |
| NMTN-02945 | SAV0706 | saeR | 780586 | 781272 | 780638 | | |
| NMTN-09648 | SAV0706 | saeR | 780586 | 781272 | 780676 | | |
| NMTN-09655 | SAV0706 | saeR | 780586 | 781272 | 780676 | | |
| NMTN-09778 | SAV0706 | saeR | 780586 | 781272 | 780676 | | |
| NMTN-11354 | SAV0706 | saeR | 780586 | 781272 | 780704 | | |
| NMTN-07103 | SAV0706 | saeR | 780586 | 781272 | 780758 | | |
| NMTN-02564 | SAV0706 | saeR | 780586 | 781272 | 780852 | | |
| NMTN-01594 | SAV0706 | saeR | 780586 | 781272 | 780870 | | |
| NMTN-12583 | SAV0706 | saeR | 780586 | 781272 | 780910 | | |
| NMTN-14421 | SAV0707 | | 781247 | 781429 | 781311 | − | hypothetical protein |
| NMTN-02705 | SAV0707 | | 781247 | 781429 | 781395 | | |
| NMTN-13902 | SAV0707 | | 781247 | 781429 | 781416 | | |
| NMTN-13878 | SAV0707 | | 781247 | 781429 | 781423 | | |
| NMTN-12351 | SAV0707 | | 781247 | 781429 | 781428 | | |
| NMTN-11666 | SAV0708 | | 782063 | 782503 | 782126 | − | hypothetical protein |
| NMTN-03947 | SAV0708 | | 782063 | 782503 | 782231 | | |
| NMTN-07547 | SAV0708 | | 782063 | 782503 | 782231 | | |
| NMTN-06932 | SAV0708 | | 782063 | 782503 | 782239 | | |
| NMTN-09654 | SAV0708 | | 782063 | 782503 | 782359 | | |
| NMTN-01776 | SAV0709 | | 782783 | 783367 | 783213 | − | hypothetical protein |
| NMTN-03711 | SAV0711 | | 784183 | 784602 | 784362 | − | 6-pyruvoyl tetrahydrobiopterin synthase |
| | | | | | | | homologue |
| NMTN-09059 | SAV0712 | | 784604 | 785272 | 784996 | − | conserved hypothetical protein |
| NMTN-11117 | SAV0713 | | 785623 | 786216 | 785657 | + | hypothetical protein |
| NMTN-06611 | SAV0713 | | 785623 | 786216 | 785673 | | |
| NMTN-12284 | SAV0713 | | 785623 | 786216 | 785845 | | |
| NMTN-06730 | SAV0713 | | 785623 | 786216 | 785936 | | |
| NMTN-07478 | SAV0713 | | 785623 | 786216 | 786175 | | |
| NMTN-02930 | SAV0714 | | 786200 | 787351 | 786289 | + | hypothetical protein |
| NMTN-11500 | SAV0714 | | 786200 | 787351 | 786289 | | |
| NMTN-08783 | SAV0714 | | 786200 | 787351 | 786441 | | |
| NMTN-14279 | SAV0714 | | 786200 | 787351 | 786697 | | |
| NMTN-09306 | SAV0714 | | 786200 | 787351 | 787346 | | |
| NMTN-04472 | SAV0715 | | 787351 | 787959 | 787351 | + | hypothetical protein |
| NMTN-00549 | SAV0715 | | 787351 | 787959 | 787503 | | |
| NMTN-10837 | SAV0715 | | 787351 | 787959 | 787520 | | |
| NMTN-06655 | SAV0715 | | 787351 | 787959 | 787702 | | |
| NMTN-03503 | SAV0715 | | 787351 | 787959 | 787883 | | |
| NMTN-05377 | SAV0717 | | 788319 | 789029 | 788486 | + | hypothetical protein |
| NMTN-13126 | SAV0718 | | 789013 | 790017 | 789206 | + | conserved hypothetical protein |
| NMTN-04130 | SAV0718 | | 789013 | 790017 | 789274 | | |
| NMTN-13658 | SAV0718 | | 789013 | 790017 | 789984 | | |
| NMTN-04390 | SAV0718 | | 789013 | 790017 | 789988 | | |
| NMTN-10194 | SAV0720 | | 792707 | 794584 | 792734 | + | hypothetical protein |
| NMTN-04908 | SAV0720 | | 792707 | 794584 | 792818 | | |
| NMTN-14663 | SAV0720 | | 792707 | 794584 | 792989 | | |
| NMTN-06242 | SAV0720 | | 792707 | 794584 | 793396 | | |
| NMTN-06521 | SAV0720 | | 792707 | 794584 | 793496 | | |
| NMTN-03480 | SAV0720 | | 792707 | 794584 | 793606 | | |
| NMTN-13091 | SAV0720 | | 792707 | 794584 | 793670 | | |
| NMTN-09483 | SAV0721 | recQ | 794596 | 796377 | 794717 | + | probable DNA helicase |
| NMTN-06980 | SAV0721 | recQ | 794596 | 796377 | 794948 | | |
| NMTN-06308 | SAV0721 | recQ | 794596 | 796377 | 795914 | | |
| NMTN-09880 | SAV0722 | | 796599 | 797576 | 796620 | + | hypothetical protein |
| NMTN-11507d | SAV0722 | | 796599 | 797576 | 796808 | | |
| NMTN-05542 | SAV0723 | | 797569 | 799083 | 798112 | + | hypothetical protein |
| NMTN-13814 | SAV0723 | | 797569 | 799083 | 798163 | | |
| NMTN-03050 | SAV0723 | | 797569 | 799083 | 798665 | | |
| NMTN-01575 | SAV0723 | | 797569 | 799083 | 798742 | | |
| NMTN-02726 | SAV0723 | | 797569 | 799083 | 798758 | | |
| NMTN-05084 | SAV0723 | | 797569 | 799083 | 798808 | | |
| NMTN-12500 | SAV0724 | | 799323 | 800381 | 799548 | + | hypothetical protein |
| NMTN-02266 | SAV0724 | | 799323 | 800381 | 799713 | | |
| NMTN-09356 | SAV0724 | | 799323 | 800381 | 799816 | | |
| NMTN-05002 | SAV0725 | | 800720 | 801262 | 800766 | + | conserved hypothetical protein |
| NMTN-13026 | SAV0725 | | 800720 | 801262 | 800978 | | |
| NMTN-07217 | SAV0725 | | 800720 | 801262 | 800982 | | |
| NMTN-00328 | SAV0726 | | 802085 | 803002 | 802516 | − | hypothetical protein |
| NMTN-09786 | SAV0726 | | 802085 | 803002 | 803006 | | |
| NMTN-10568 | SAV0727 | | 803272 | 804777 | 803476 | − | hypothetical protein |
| NMTN-10444 | SAV0727 | | 803272 | 804777 | 803724 | | |
| NMTN-10634 | SAV0727 | | 803272 | 804777 | 804077 | | |
| NMTN-09563 | SAV0727 | | 803272 | 804777 | 804314 | | |
| NMTN-09164 | SAV0727 | | 803272 | 804777 | 804317 | | |
| NMTN-11758 | SAV0727 | | 803272 | 804777 | 804438 | | |
| NMTN-12450 | SAV0727 | | 803272 | 804777 | 804449 | | |
| NMTN-13087 | SAV0727 | | 803272 | 804777 | 804541 | | |
| NMTN-08154 | SAV0727 | | 803272 | 804777 | 804624 | | |
| NMTN-04880 | SAV0727 | | 803272 | 804777 | 804625 | | |
| NMTN-10566 | SAV0727 | | 803272 | 804777 | 804780 | | |
| NMTN-13548 | SAV0728 | | 805118 | 805618 | 805115 | − | conserved hypothetical protein |
| NMTN-10400 | SAV0728 | | 805118 | 805618 | 805189 | | |
| NMTN-10547 | SAV0728 | | 805118 | 805618 | 805503 | | |
| NMTN-02913 | SAV0729 | | 805638 | 806504 | 806071 | − | conserved hypothetical protein |
| NMTN-14605 | SAV0729 | | 805638 | 806504 | 806227 | | |
| NMTN-02988 | SAV0733 | | 811235 | 812206 | 811562 | + | hypothetical protein |
| NMTN-02275 | SAV0733 | | 811235 | 812206 | 811949 | | |
| NMTN-02670 | SAV0733 | | 811235 | 812206 | 812087 | | |
| NMTN-13846 | SAV0734 | | 812193 | 813149 | 812255 | + | hypothetical protein |
| NMTN-02772 | SAV0734 | | 812193 | 813149 | 812511 | | |
| NMTN-08763 | SAV0734 | | 812193 | 813149 | 812644 | | |
| NMTN-09241 | SAV0734 | | 812193 | 813149 | 812692 | | |
| NMTN-13262 | SAV0734 | | 812193 | 813149 | 812754 | | |
| NMTN-02619 | SAV0734 | | 812193 | 813149 | 812792 | | |
| NMTN-13566 | SAV0734 | | 812193 | 813149 | 813024 | | |
| NMTN-14644 | SAV0734 | | 812193 | 813149 | 813084 | | |
| NMTN-08880 | SAV0735 | | 813146 | 813907 | 813284 | + | hypothetical protein |
| NMTN-12408d | SAV0735 | | 813146 | 813907 | 813639 | | |
| NMTN-14654 | SAV0736 | | 814026 | 815054 | 814695 | + | lipoprotein |
| NMTN-00012 | SAV0736 | | 814026 | 815054 | 815008 | | |
| NMTN-01999 | SAV0737 | | 815371 | 815685 | 815448 | − | conserved hypothetical protein |
| NMTN-13315 | SAV0737 | | 815371 | 815685 | 815684 | | |
| NMTN-10613 | SAV0740 | | 817391 | 818269 | 817439 | + | hypothetical protein |
| NMTN-05052 | SAV0740 | | 817391 | 818269 | 817665 | | |
| NMTN-02004 | SAV0740 | | 817391 | 818269 | 817725 | | |
| NMTN-05187 | SAV0740 | | 817391 | 818269 | 818130 | | |
| NMTN-07067 | SAV0742 | | 819167 | 820291 | 819466 | + | hypothetical protein |
| NMTN-09297 | SAV0742 | | 819167 | 820291 | 819775 | | |
| NMTN-06060 | SAV0743 | pepT | 820478 | 821704 | 821055 | − | aminotripeptidase |
| NMTN-00601 | SAV0743 | pepT | 820478 | 821704 | 821118 | | |
| NMTN-02418 | SAV0743 | pepT | 820478 | 821704 | 821121 | | |
| NMTN-04725 | SAV0743 | pepT | 820478 | 821704 | 821634 | | |
| NMTN-05140 | SAV0743 | pepT | 820478 | 821704 | 821672 | | |
| NMTN-09363 | SAV0744 | | 821718 | 822212 | 821799 | − | conserved hypothetical protein |
| NMTN-14757d | SAV0745 | | 822230 | 822991 | 822361 | − | conserved hypothetical protein |
| NMTN-05370 | SAV0745 | | 822230 | 822991 | 822940 | | |
| NMTN-01804 | SAV0746 | | 823187 | 824257 | 823885 | − | conserved hypothetical protein |
| NMTN-01849 | SAV0746 | | 823187 | 824257 | 823885 | | |
| NMTN-14380 | SAV0746 | | 823187 | 824257 | 824244 | | |
| NMTN-13309 | SAV0748 | | 825795 | 826436 | 826294 | − | conserved hypothetical protein |
| NMTN-13833 | SAV0749 | | 826580 | 827446 | 826605 | + | conserved hypothetical protein |
| NMTN-12261 | SAV0749 | | 826580 | 827446 | 826868 | | |
| NMTN-11101 | SAV0750 | | 827990 | 828892 | 828122 | + | hypothetical protein |
| NMTN-00670 | SAV0750 | | 827990 | 828892 | 828323 | | |
| NMTN-01492 | SAV0750 | | 827990 | 828892 | 828534 | | |
| NMTN-14691 | SAV0750 | | 827990 | 828892 | 828562 | | |
| NMTN-13522 | SAV0751 | | 828885 | 829559 | 828940 | + | hypothetical protein |
| NMTN-11533 | SAV0751 | | 828885 | 829559 | 829009 | | |
| NMTN-07108 | SAV0751 | | 828885 | 829559 | 829468 | | |
| NMTN-08563 | SAV0755 | | 834971 | 835810 | 835126 | + | conserved hypothetical protein |
| NMTN-12918 | SAV0755 | | 834971 | 835810 | 835129 | | |
| NMTN-08563 | SAV0755 | | 834971 | 835810 | 835139 | | |
| NMTN-03632 | SAV0755 | | 834971 | 835810 | 835153 | | |
| NMTN-00872 | SAV0755 | | 834971 | 835810 | 835356 | | |
| NMTN-00148 | SAV0755 | | 834971 | 835810 | 835553 | | |
| NMTN-00513 | SAV0755 | | 834971 | 835810 | 835553 | | |
| NMTN-03587 | SAV0755 | | 834971 | 835810 | 835625 | | |
| NMTN-11364 | SAV0758 | uvrB | 837130 | 839121 | 837128 | + | exinuclease ABC subunit B |
| NMTN-11672 | SAV0758 | uvrB | 837130 | 839121 | 837361 | | |
| NMTN-06591 | SAV0758 | uvrB | 837130 | 839121 | 837362 | | |
| NMTN-13361 | SAV0758 | uvrB | 837130 | 839121 | 837547 | | |
| NMTN-09516 | SAV0758 | uvrB | 837130 | 839121 | 838194 | | |
| NMTN-13867 | SAV0758 | uvrB | 837130 | 839121 | 838886 | | |
| NMTN-12378 | SAV0759 | uvrA | 839129 | 841975 | 839390 | + | exinuclease ABC subunit A |
| NMTN-14666 | SAV0759 | uvrA | 839129 | 841975 | 839660 | | |
| NMTN-07896 | SAV0759 | uvrA | 839129 | 841975 | 840308 | | |
| NMTN-06628 | SAV0759 | uvrA | 839129 | 841975 | 840391 | | |
| NMTN-09244 | SAV0759 | uvrA | 839129 | 841975 | 840612 | | |
| NMTN-05522 | SAV0759 | uvrA | 839129 | 841975 | 840806 | | |
| NMTN-02611 | SAV0759 | uvrA | 839129 | 841975 | 841234 | | |
| NMTN-13319 | SAV0759 | uvrA | 839129 | 841975 | 841256 | | |
| NMTN-08136 | SAV0759 | uvrA | 839129 | 841975 | 841697 | | |
| NMTN-00560 | SAV0759 | uvrA | 839129 | 841975 | 841725 | | |
| NMTN-00056 | SAV0759 | uvrA | 839129 | 841975 | 841728 | | |
| NMTN-11142 | SAV0759 | uvrA | 839129 | 841975 | 841821 | | |
| NMTN-04343 | SAV0759 | uvrA | 839129 | 841975 | 841863 | | |
| NMTN-04223 | SAV0760 | hprK | 842639 | 843571 | 842678 | + | HPr kinase/phosphatase |
| NMTN-14025 | SAV0760 | hprK | 842639 | 843571 | 842729 | | |
| NMTN-13228 | SAV0760 | hprK | 842639 | 843571 | 842741 | | |
| NMTN-06976 | SAV0760 | hprK | 842639 | 843571 | 842961 | | |
| NMTN-15201 | SAV0761 | lgt | 843577 | 844416 | 843594 | + | prolipoprotein diacylglyceryl transferase |
| NMTN-15206 | SAV0761 | lgt | 843577 | 844416 | 843594 | | |
| NMTN-15207 | SAV0761 | lgt | 843577 | 844416 | 843596 | | |
| NMTN-07358 | SAV0761 | lgt | 843577 | 844416 | 843601 | | |
| NMTN-13881 | SAV0761 | lgt | 843577 | 844416 | 843601 | | |
| NMTN-03114 | SAV0761 | lgt | 843577 | 844416 | 843733 | | |
| NMTN-00106 | SAV0761 | lgt | 843577 | 844416 | 844079 | | |
| NMTN-09213 | SAV0761 | lgt | 843577 | 844416 | 844087 | | |
| NMTN-03713 | SAV0761 | lgt | 843577 | 844416 | 844321 | | |
| NMTN-02399 | SAV0762 | | 844424 | 844909 | 844691 | + | hypothetical protein |
| NMTN-05151 | SAV0763 | | 844917 | 846356 | 845539 | + | conserved hypothetical protein |
| NMTN-11709 | SAV0763 | | 844917 | 846356 | 846007 | | |
| NMTN-06055 | SAV0763 | | 844917 | 846356 | 846316 | | |
| NMTN-06657 | SAV0763 | | 844917 | 846356 | 846319 | | |
| NMTN-08114 | SAV0765 | | 848303 | 849214 | 848939 | + | conserved hypothetical protein |
| NMTN-13116 | SAV0768 | clpP | 851825 | 852412 | 851966 | + | ATP-dependent Clp protease proteolytic |
| | | | | | | | subunit homologue |
| NMTN-07860 | SAV0769 | | 852655 | 853557 | 852998 | − | hypothetical protein |
| NMTN-11973 | SAV0769 | | 852655 | 853557 | 853241 | | |
| NMTN-00650 | SAV0769 | | 852655 | 853557 | 853250 | | |
| NMTN-00102 | SAV0769 | | 852655 | 853557 | 853349 | | |
| NMTN-11836 | SAV0769 | | 852655 | 853557 | 853421 | | |
| NMTN-03043 | SAV0769 | | 852655 | 853557 | 853423 | | |
| NMTN-15277 | SAV0770 | | 854260 | 854889 | 854609 | + | conserved hypothetical protein |
| NMTN-03118 | SAV0775 | pgm | 860104 | 861621 | 860211 | + | 2,3-diphosphoglycerate-independent |
| | | | | | | | phosphoglycerate mutase |
| NMTN-13775 | SAV0775 | pgm | 860104 | 861621 | 860211 | | |
| NMTN-06684 | SAV0775 | pgm | 860104 | 861621 | 860551 | | |
| NMTN-14656 | SAV0775 | pgm | 860104 | 861621 | 860958 | | |
| NMTN-05319 | SAV0775 | pgm | 860104 | 861621 | 860976 | | |
| NMTN-09208 | SAV0775 | pgm | 860104 | 861621 | 861249 | | |
| NMTN-14436 | SAV0779 | | 864268 | 865008 | 864370 | + | carboxyesterase precursor homologue |
| NMTN-14534 | | truncated- | 874698 | 875576 | 875336 | | |
| | | SA | | | | | |
| NMTN-04111 | SAV0800 | | 878067 | 878636 | 878221 | + | hypothetical protein |
| NMTN-06708 | SAV0803 | | 880845 | 881759 | 881232 | + | hypothetical protein |
| NMTN-12006 | SAV0803 | | 880845 | 881759 | 881232 | | |
| NMTN-03793 | SAV0803 | | 880845 | 881759 | 881412 | | |
| NMTN-12000 | SAV0804 | | 881817 | 882665 | 882068 | + | hypothetical protein |
| NMTN-06738 | SAV0804 | | 881817 | 882665 | 882130 | | |
| NMTN-02513 | SAV0804 | | 881817 | 882665 | 882190 | | |
| NMTN-10493 | SAV0805 | | 883041 | 883181 | 883071 | + | hypothetical protein |
| NMTN-02360 | SAV0805 | | 883041 | 883181 | 883140 | | |
| NMTN-02656 | SAV0805 | | 883041 | 883181 | 883140 | | |
| NMTN-12858 | SAV0808 | | 884407 | 885135 | 885101 | − | conserved hypothetical protein |
| NMTN-11273 | SAV0809 | | 885733 | 886254 | 885927 | + | hypothetical protein |
| NMTN-09646 | SAV0809 | | 885733 | 886254 | 886092 | | |
| NMTN-06882 | SAV0810 | | 886393 | 886923 | 886892 | + | conserved hypothetical protein |
| NMTN-07401 | SAV0811 | fnb | 887186 | 889993 | 888251 | + | fibrinogen-binding protein |
| NMTN-08738 | SAV0811 | fnb | 887186 | 889993 | 888255 | | |
| NMTN-07317 | SAV0811 | fnb | 887186 | 889993 | 888481 | | |
| NMTN-05280 | SAV0811 | fnb | 887186 | 889993 | 888645 | | |
| NMTN-06933 | SAV0812 | | 890214 | 891716 | 891568 | + | hypothetical protein |
| NMTN-07970 | SAV0813 | ssp | 892069 | 893091 | 892618 | + | extracellular ECM and plasma binding |
| | | | | | | | protein |
| NMTN-00036 | SAV0813 | ssp | 892069 | 893091 | 892625 | | |
| NMTN-09415 | SAV0813 | ssp | 892069 | 893091 | 892652 | | |
| NMTN-12513 | SAV0813 | ssp | 892069 | 893091 | 893049 | | |
| NMTN-09823 | SAV0815 | nuc | 894288 | 894974 | 894306 | + | staphylococcal nuclease |
| NMTN-10057 | SAV0815 | nuc | 894288 | 894974 | 894340 | | |
| NMTN-03877 | SAV0815 | nuc | 894288 | 894974 | 894342 | | |
| NMTN-14464 | SAV0815 | nuc | 894288 | 894974 | 894521 | | |
| NMTN-11524 | SAV0815 | nuc | 894288 | 894974 | 894523 | | |
| NMTN-12201 | SAV0815 | nuc | 894288 | 894974 | 894776 | | |
| NMTN-09519 | SAV0819 | | 896675 | 897244 | 896675 | − | conserved hypothetical protein |
| NMTN-07534d | SAV0822 | | 898250 | 898453 | 898341 | + | hypothetical protein |
| NMTN-11151 | SAV0825 | | 899565 | 900182 | 899928 | − | conserved hypothetical protein |
| NMTN-11590 | SAV0825 | | 899565 | 900182 | 900005 | | |
| NMTN-11279 | SAV0825 | | 899565 | 900182 | 900072 | | |
| NMTN-06610 | SAV0826 | | 900337 | 900831 | 900384 | − | hypothetical protein |
| NMTN-12871 | SAV0830 | | 902599 | 903138 | 902608 | + | conserved hypothetical protein |
| NMTN-15355 | SAV0830 | | 902599 | 903138 | 902695 | | |
| NMTN-06050 | SAV0830 | | 902599 | 903138 | 903069 | | |
| NMTN-07446 | SAV0830 | | 902599 | 903138 | 903121 | | |
| NMTN-06228 | SAV0830 | | 902599 | 903138 | 903142 | | |
| NMTN-02894 | SAV0835 | | 906501 | 906887 | 906612 | + | conserved hypothetical protein |
| NMTN-05142 | SAV0837 | | 907426 | 908451 | 907633 | + | ABC transporter ATP-binding protein |
| | | | | | | | homologue |
| NMTN-08443 | SAV0837 | | 907426 | 908451 | 908213 | | |
| NMTN-02404 | SAV0838 | | 908444 | 909139 | 908741 | + | conserved hypothetical protein |
| NMTN-00555 | SAV0838 | | 908444 | 909139 | 909016 | | |
| NMTN-12909 | SAV0839 | | 909157 | 909978 | 909416 | + | conserved hypothetical protein |
| NMTN-04279 | SAV0841 | | 910760 | 911611 | 911069 | − | conserved hypothetical protein |
| NMTN-03788 | SAV0845 | | 915451 | 915915 | 915875 | + | hypothetical protein |
| NMTN-09814 | SAV0848 | int | 918692 | 918871 | 918712 | + | excisionase |
| NMTN-11204 | SAV0848 | int | 918692 | 918871 | 918724 | | |
| NMTN-13398 | SAV0848 | int | 918692 | 918871 | 918724 | | |
| NMTN-11204 | SAV0848 | int | 918692 | 918871 | 918724 | | |
| NMTN-09031 | SAV0848 | int | 918692 | 918871 | 918753 | | |
| NMTN-08829 | SAV0848 | int | 918692 | 918871 | 918864 | | |
| NMTN-08829 | SAV0849 | | 918851 | 919783 | 918864 | − | hypothetical protein |
| NMTN-06091 | SAV0849 | | 918851 | 919783 | 918984 | | |
| NMTN-12286 | SAV0849 | | 918851 | 919783 | 919060 | | |
| NMTN-12689 | SAV0849 | | 918851 | 919783 | 919144 | | |
| NMTN-10197 | SAV0849 | | 918851 | 919783 | 919217 | | |
| NMTN-01443 | SAV0849 | | 918851 | 919783 | 919338 | | |
| NMTN-07294 | SAV0849 | | 918851 | 919783 | 919469 | | |
| NMTN-09514 | SAV0849 | | 918851 | 919783 | 919505 | | |
| NMTN-00029 | SAV0849 | | 918851 | 919783 | 919507 | | |
| NMTN-00225 | SAV0849 | | 918851 | 919783 | 919578 | | |
| NMTN-07262 | SAV0855 | | 921461 | 922240 | 921524 | | |
| NMTN-03416 | SAV0855 | | 921461 | 922240 | 921803 | + | hypothetical protein |
| NMTN-05572 | SAV0855 | | 921461 | 922240 | 921890 | | |
| NMTN-00340 | SAV0855 | | 921461 | 922240 | 921935 | | |
| NMTN-02139 | SAV0855 | | 921461 | 922240 | 922032 | | |
| NMTN-15272d | SAV0855 | | 921461 | 922240 | 922212 | | |
| NMTN-12671 | SAV0858 | | 922670 | 923317 | 923315 | − | hypothetical protein |
| NMTN-04000 | SAV0861 | | 924300 | 924434 | 924295 | − | hypothetical protein |
| NMTN-09143 | SAV0861 | | 924300 | 924434 | 924359 | | |
| NMTN-12478 | SAV0862 | | 924433 | 924654 | 924542 | + | hypothetical protein |
| NMTN-06544 | SAV0862 | | 924433 | 924654 | 924654 | | |
| NMTN-06544 | SAV0863 | | 924647 | 924769 | 924654 | + | hypothetical protein |
| NMTN-12038 | SAV0866 | | 926259 | 926933 | 926732 | + | hypothetical protein |
| NMTN-08772 | SAV0866 | | 926259 | 926933 | 926793 | | |
| NMTN-07325 | SAV0868 | | 927419 | 928150 | 927412 | + | hypothetical protein |
| NMTN-12985 | SAV0868 | | 927419 | 928150 | 927412 | | |
| NMTN-06487 | SAV0874 | | 930315 | 930563 | 930509 | + | hypothetical protein |
| NMTN-04743 | SAV0875 | | 930575 | 930826 | 930676 | + | hypothetical protein |
| NMTN-06711 | SAV0876 | | 930819 | 931355 | 930895 | + | hypothetical protein |
| NMTN-03004 | SAV0876 | | 930819 | 931355 | 931262 | | |
| NMTN-01478 | SAV0877 | | 931392 | 931598 | 931450 | + | hypothetical protein |
| NMTN-12253 | SAV0881 | | 932208 | 932594 | 932330 | + | hypothetical protein |
| NMTN-09411 | SAV0881 | | 932208 | 932594 | 932405 | | |
| NMTN-08472 | SAV0881 | | 932208 | 932594 | 932427 | | |
| NMTN-04372 | SAV0881 | | 932208 | 932594 | 932448 | | |
| NMTN-14628 | SAV0881 | | 932208 | 932594 | 932481 | | |
| NMTN-12793 | SAV0881 | | 932208 | 932594 | 932499 | | |
| NMTN-09388 | SAV0881 | | 932208 | 932594 | 932530 | | |
| NMTN-05470 | SAV0881 | | 932208 | 932594 | 932536 | | |
| NMTN-03184 | SAV0881 | | 932208 | 932594 | 932553 | | |
| NMTN-11073 | SAV0882 | | 932594 | 932767 | 932640 | + | int gene activator RinB |
| NMTN-03060 | SAV0882 | | 932594 | 932767 | 932673 | | |
| NMTN-01698 | SAV0882 | | 932594 | 932767 | 932699 | | |
| NMTN-00024 | SAV0883 | | 932825 | 932914 | 932879 | + | hypothetical protein |
| NMTN-05312 | SAV0885 | | 933535 | 933975 | 933567 | + | hypothetical protein |
| NMTN-08673 | SAV0885 | | 933535 | 933975 | 933567 | | |
| NMTN-08726 | SAV0885 | | 933535 | 933975 | 933680 | | |
| NMTN-11632 | SAV0885 | | 933535 | 933975 | 933837 | | |
| NMTN-10789 | SAV0885 | | 933535 | 933975 | 933889 | | |
| NMTN-06264 | SAV0886 | | 933962 | 935239 | 934232 | + | hypothetical protein |
| NMTN-08868 | SAV0886 | | 933962 | 935239 | 934289 | | |
| NMTN-06516 | SAV0886 | | 933962 | 935239 | 934434 | | |
| NMTN-00046 | SAV0886 | | 933962 | 935239 | 934485 | | |
| NMTN-14674 | SAV0886 | | 933962 | 935239 | 934539 | | |
| NMTN-08440 | SAV0886 | | 933962 | 935239 | 934793 | | |
| NMTN-08764 | SAV0886 | | 933962 | 935239 | 934824 | | |
| NMTN-08843c | SAV0886 | | 933962 | 935239 | 934870 | | |
| NMTN-12881d | SAV0886 | | 933962 | 935239 | 934881 | | |
| NMTN-04036 | SAV0886 | | 933962 | 935239 | 935128 | | |
| NMTN-05193 | SAV0887 | | 935250 | 936785 | 935339 | + | hypothetical protein |
| NMTN-10805 | SAV0887 | | 935250 | 936785 | 935428 | | |
| NMTN-14481 | SAV0887 | | 935250 | 936785 | 935458 | | |
| NMTN-03820 | SAV0887 | | 935250 | 936785 | 935528 | | |
| NMTN-01646 | SAV0887 | | 935250 | 936785 | 935540 | | |
| NMTN-08108 | SAV0887 | | 935250 | 936785 | 935624 | | |
| NMTN-01676 | SAV0887 | | 935250 | 936785 | 935659 | | |
| NMTN-06181 | SAV0887 | | 935250 | 936785 | 935775 | | |
| NMTN-06090 | SAV0887 | | 935250 | 936785 | 935797 | | |
| NMTN-13389 | SAV0887 | | 935250 | 936785 | 935800 | | |
| NMTN-03684 | SAV0887 | | 935250 | 936785 | 935878 | | |
| NMTN-01869 | SAV0887 | | 935250 | 936785 | 935903 | | |
| NMTN-03001 | SAV0887 | | 935250 | 936785 | 935980 | | |
| NMTN-01729 | SAV0887 | | 935250 | 936785 | 936012 | | |
| NMTN-12852 | SAV0887 | | 935250 | 936785 | 936080 | | |
| NMTN-01827 | SAV0887 | | 935250 | 936785 | 936257 | | |
| NMTN-03204 | SAV0887 | | 935250 | 936785 | 936260 | | |
| NMTN-02736 | SAV0887 | | 935250 | 936785 | 936621 | | |
| NMTN-08106 | SAV0887 | | 935250 | 936785 | 936713 | | |
| NMTN-00084 | SAV0888 | | 936792 | 937787 | 937091 | + | hypothetical protein |
| NMTN-10573 | SAV0888 | | 936792 | 937787 | 937094 | | |
| NMTN-10565 | SAV0888 | | 936792 | 937787 | 937096 | | |
| NMTN-06149 | SAV0888 | | 936792 | 937787 | 937125 | | |
| NMTN-02714 | SAV0888 | | 936792 | 937787 | 937278 | | |
| NMTN-01533 | SAV0888 | | 936792 | 937787 | 937282 | | |
| NMTN-07483 | SAV0888 | | 936792 | 937787 | 937480 | | |
| NMTN-02251 | SAV0888 | | 936792 | 937787 | 937568 | | |
| NMTN-11790 | SAV0888 | | 936792 | 937787 | 937568 | | |
| NMTN-06287 | SAV0888 | | 936792 | 937787 | 937571 | | |
| NMTN-06600 | SAV0888 | | 936792 | 937787 | 937571 | | |
| NMTN-04423 | SAV0888 | | 936792 | 937787 | 937587 | | |
| NMTN-03685 | SAV0889 | | 937860 | 938030 | 937893 | + | hypothetical protein |
| NMTN-00176 | SAV0889 | | 937860 | 938030 | 938011 | | |
| NMTN-00294 | SAV0890 | | 938165 | 938779 | 938229 | + | hypothetical protein |
| NMTN-00497 | SAV0890 | | 938165 | 938779 | 938483 | | |
| NMTN-09919 | SAV0891 | | 938793 | 939101 | 938973 | + | hypothetical protein |
| NMTN-08474 | SAV0891 | | 938793 | 939101 | 939096 | | |
| NMTN-08478 | SAV0891 | | 938793 | 939101 | 939096 | | |
| NMTN-08479 | SAV0891 | | 938793 | 939101 | 939096 | | |
| NMTN-08480 | SAV0891 | | 938793 | 939101 | 939096 | | |
| NMTN-08483 | SAV0891 | | 938793 | 939101 | 939096 | | |
| NMTN-08484 | SAV0891 | | 938793 | 939101 | 939096 | | |
| NMTN-08485 | SAV0891 | | 938793 | 939101 | 939096 | | |
| NMTN-08486 | SAV0891 | | 938793 | 939101 | 939096 | | |
| NMTN-08487 | SAV0891 | | 938793 | 939101 | 939096 | | |
| NMTN-08488 | SAV0891 | | 938793 | 939101 | 939096 | | |
| NMTN-08489 | SAV0891 | | 938793 | 939101 | 939096 | | |
| NMTN-08493 | SAV0891 | | 938793 | 939101 | 939096 | | |
| NMTN-08497 | SAV0891 | | 938793 | 939101 | 939096 | | |
| NMTN-08498 | SAV0891 | | 938793 | 939101 | 939096 | | |
| NMTN-08501 | SAV0891 | | 938793 | 939101 | 939096 | | |
| NMTN-08502 | SAV0891 | | 938793 | 939101 | 939096 | | |
| NMTN-08503 | SAV0891 | | 938793 | 939101 | 939096 | | |
| NMTN-08505 | SAV0891 | | 938793 | 939101 | 939096 | | |
| NMTN-08507 | SAV0891 | | 938793 | 939101 | 939096 | | |
| NMTN-08511 | SAV0891 | | 938793 | 939101 | 939096 | | |
| NMTN-08512 | SAV0891 | | 938793 | 939101 | 939096 | | |
| NMTN-08513 | SAV0891 | | 938793 | 939101 | 939096 | | |
| NMTN-08516 | SAV0891 | | 938793 | 939101 | 939096 | | |
| NMTN-08519 | SAV0891 | | 938793 | 939101 | 939096 | | |
| NMTN-08520 | SAV0891 | | 938793 | 939101 | 939096 | | |
| NMTN-08521 | SAV0891 | | 938793 | 939101 | 939096 | | |
| NMTN-08499 | SAV0892 | | 939233 | 939766 | 939291 | + | hypothetical protein |
| NMTN-15214 | SAV0892 | | 939233 | 939766 | 939441 | | |
| NMTN-00667 | SAV0892 | | 939233 | 939766 | 939470 | | |
| NMTN-04129 | SAV0892 | | 939233 | 939766 | 939675 | | |
| NMTN-11287 | SAV0893 | | 939788 | 940075 | 939853 | + | hypothetical protein |
| NMTN-14537 | SAV0893 | | 939788 | 940075 | 940083 | | |
| NMTN-14537 | SAV0894 | | 940084 | 940416 | 940083 | + | hypothetical protein |
| NMTN-02185 | SAV0894 | | 940084 | 940416 | 940238 | | |
| NMTN-03655 | SAV0895 | | 940413 | 940715 | 940515 | + | hypothetical protein |
| NMTN-08496 | SAV0896 | | 940715 | 941062 | 940751 | + | hypothetical protein |
| NMTN-06748 | SAV0896 | | 940715 | 941062 | 940897 | | |
| NMTN-02232 | SAV0896 | | 940715 | 941062 | 941008 | | |
| NMTN-06484 | SAV0897 | | 941074 | 941457 | 941141 | + | hypothetical protein |
| NMTN-12160 | SAV0897 | | 941074 | 941457 | 941393 | | |
| NMTN-03687 | SAV0897 | | 941074 | 941457 | 941399 | | |
| NMTN-10971 | SAV0898 | | 941476 | 942057 | 941507 | + | hypothetical protein |
| NMTN-04734 | SAV0898 | | 941476 | 942057 | 941843 | | |
| NMTN-06680 | SAV0898 | | 941476 | 942057 | 942041 | | |
| NMTN-09552 | SAV0900 | | 942604 | 942858 | 942764 | + | hypothetical protein |
| NMTN-14560 | SAV0900 | | 942604 | 942858 | 942833 | | |
| NMTN-09067 | SAV0900 | | 942604 | 942858 | 942869 | | |
| NMTN-10046 | SAV0901 | | 942875 | 946339 | 943089 | + | hypothetical protein |
| NMTN-10062 | SAV0901 | | 942875 | 946339 | 943089 | | |
| NMTN-03708 | SAV0901 | | 942875 | 946339 | 943127 | | |
| NMTN-06253 | SAV0901 | | 942875 | 946339 | 943356 | | |
| NMTN-12796 | SAV0901 | | 942875 | 946339 | 943458 | | |
| NMTN-13838 | SAV0901 | | 942875 | 946339 | 943473 | | |
| NMTN-06620 | SAV0901 | | 942875 | 946339 | 943622 | | |
| NMTN-14523 | SAV0901 | | 942875 | 946339 | 943626 | | |
| NMTN-09458 | SAV0901 | | 942875 | 946339 | 943817 | | |
| NMTN-01917 | SAV0901 | | 942875 | 946339 | 944127 | | |
| NMTN-07916 | SAV0901 | | 942875 | 946339 | 944310 | | |
| NMTN-12335 | SAV0901 | | 942875 | 946339 | 944461 | | |
| NMTN-14047 | SAV0901 | | 942875 | 946339 | 944461 | | |
| NMTN-13605 | SAV0901 | | 942875 | 946339 | 944508 | | |
| NMTN-14483 | SAV0901 | | 942875 | 946339 | 944673 | | |
| NMTN-14818 | SAV0901 | | 942875 | 946339 | 944673 | | |
| NMTN-14850 | SAV0901 | | 942875 | 946339 | 944673 | | |
| NMTN-14858 | SAV0901 | | 942875 | 946339 | 944673 | | |
| NMTN-00064 | SAV0901 | | 942875 | 946339 | 944681 | | |
| NMTN-13611 | SAV0901 | | 942875 | 946339 | 944694 | | |
| NMTN-14100 | SAV0901 | | 942875 | 946339 | 944694 | | |
| NMTN-10885 | SAV0901 | | 942875 | 946339 | 944862 | | |
| NMTN-03865 | SAV0901 | | 942875 | 946339 | 944936 | | |
| NMTN-03873 | SAV0901 | | 942875 | 946339 | 944936 | | |
| NMTN-13581 | SAV0901 | | 942875 | 946339 | 945173 | | |
| NMTN-11192 | SAV0901 | | 942875 | 946339 | 945311 | | |
| NMTN-14524 | SAV0901 | | 942875 | 946339 | 945348 | | |
| NMTN-06240 | SAV0901 | | 942875 | 946339 | 945534 | | |
| NMTN-08008 | SAV0901 | | 942875 | 946339 | 945656 | | |
| NMTN-04159 | SAV0901 | | 942875 | 946339 | 945741 | | |
| NMTN-08089 | SAV0901 | | 942875 | 946339 | 946111 | | |
| NMTN-10887 | SAV0901 | | 942875 | 946339 | 946134 | | |
| NMTN-00607 | SAV0901 | | 942875 | 946339 | 946161 | | |
| NMTN-13200 | SAV0901 | | 942875 | 946339 | 946196 | | |
| NMTN-07920 | SAV0901 | | 942875 | 946339 | 946349 | | |
| NMTN-07920 | SAV0902 | | 946352 | 947299 | 946349 | + | hypothetical protein |
| NMTN-07215 | SAV0902 | | 946352 | 947299 | 946381 | | |
| NMTN-00226 | SAV0902 | | 946352 | 947299 | 946394 | | |
| NMTN-12885 | SAV0902 | | 946352 | 947299 | 946430 | | |
| NMTN-02791 | SAV0902 | | 946352 | 947299 | 946614 | | |
| NMTN-06112 | SAV0902 | | 946352 | 947299 | 946614 | | |
| NMTN-00465 | SAV0902 | | 946352 | 947299 | 946615 | | |
| NMTN-07271 | SAV0902 | | 946352 | 947299 | 946694 | | |
| NMTN-09632 | SAV0902 | | 946352 | 947299 | 946794 | | |
| NMTN-06860 | SAV0902 | | 946352 | 947299 | 946855 | | |
| NMTN-04745 | SAV0902 | | 946352 | 947299 | 947007 | | |
| NMTN-06483 | SAV0902 | | 946352 | 947299 | 947013 | | |
| NMTN-11378d | SAV0903 | | 947308 | 949209 | 947317 | + | hypothetical protein |
| NMTN-08824 | SAV0903 | | 947308 | 949209 | 947359 | | |
| NMTN-10528 | SAV0903 | | 947308 | 949209 | 947372 | | |
| NMTN-10630 | SAV0903 | | 947308 | 949209 | 947423 | | |
| NMTN-02017 | SAV0903 | | 947308 | 949209 | 947506 | | |
| NMTN-12635 | SAV0903 | | 947308 | 949209 | 947510 | | |
| NMTN-07759 | SAV0903 | | 947308 | 949209 | 947813 | | |
| NMTN-03000 | SAV0903 | | 947308 | 949209 | 948187 | | |
| NMTN-12740 | SAV0903 | | 947308 | 949209 | 948200 | | |
| NMTN-06191 | SAV0903 | | 947308 | 949209 | 948238 | | |
| NMTN-12823 | SAV0903 | | 947308 | 949209 | 948260 | | |
| NMTN-02845 | SAV0903 | | 947308 | 949209 | 948318 | | |
| NMTN-00701 | SAV0903 | | 947308 | 949209 | 948497 | | |
| NMTN-05339 | SAV0903 | | 947308 | 949209 | 948497 | | |
| NMTN-13033 | SAV0903 | | 947308 | 949209 | 948766 | | |
| NMTN-03165 | SAV0903 | | 947308 | 949209 | 948785 | | |
| NMTN-11107 | SAV0903 | | 947308 | 949209 | 948993 | | |
| NMTN-11438 | SAV0903 | | 947308 | 949209 | 949095 | | |
| NMTN-01552 | SAV0904 | | 949224 | 951134 | 949278 | + | hypothetical protein |
| NMTN-07940 | SAV0904 | | 949224 | 951134 | 949302 | | |
| NMTN-12916 | SAV0904 | | 949224 | 951134 | 949302 | | |
| NMTN-01705 | SAV0904 | | 949224 | 951134 | 949372 | | |
| NMTN-08825 | SAV0904 | | 949224 | 951134 | 949425 | | |
| NMTN-03093 | SAV0904 | | 949224 | 951134 | 949439 | | |
| NMTN-07500 | SAV0904 | | 949224 | 951134 | 949524 | | |
| NMTN-13428 | SAV0904 | | 949224 | 951134 | 949699 | | |
| NMTN-02959 | SAV0904 | | 949224 | 951134 | 949702 | | |
| NMTN-11185 | SAV0904 | | 949224 | 951134 | 949784 | | |
| NMTN-06128 | SAV0904 | | 949224 | 951134 | 949804 | | |
| NMTN-07585 | SAV0904 | | 949224 | 951134 | 949887 | | |
| NMTN-07585 | SAV0904 | | 949224 | 951134 | 949887 | | |
| NMTN-06299 | SAV0904 | | 949224 | 951134 | 949967 | | |
| NMTN-03953 | SAV0904 | | 949224 | 951134 | 950001 | | |
| NMTN-05531 | SAV0904 | | 949224 | 951134 | 950001 | | |
| NMTN-04146 | SAV0904 | | 949224 | 951134 | 950020 | | |
| NMTN-05044 | SAV0904 | | 949224 | 951134 | 950020 | | |
| NMTN-12313 | SAV0904 | | 949224 | 951134 | 950041 | | |
| NMTN-03621 | SAV0904 | | 949224 | 951134 | 950160 | | |
| NMTN-03900 | SAV0904 | | 949224 | 951134 | 950240 | | |
| NMTN-03908 | SAV0904 | | 949224 | 951134 | 950240 | | |
| NMTN-03910 | SAV0904 | | 949224 | 951134 | 950240 | | |
| NMTN-06261 | SAV0904 | | 949224 | 951134 | 950240 | | |
| NMTN-14057 | SAV0904 | | 949224 | 951134 | 950251 | | |
| NMTN-06231 | SAV0904 | | 949224 | 951134 | 950286 | | |
| NMTN-14603 | SAV0904 | | 949224 | 951134 | 950588 | | |
| NMTN-10647 | SAV0904 | | 949224 | 951134 | 950620 | | |
| NMTN-03625 | SAV0904 | | 949224 | 951134 | 950872 | | |
| NMTN-03987 | SAV0904 | | 949224 | 951134 | 950913 | | |
| NMTN-00864 | SAV0904 | | 949224 | 951134 | 950994 | | |
| NMTN-09341 | SAV0904 | | 949224 | 951134 | 951013 | | |
| NMTN-00876 | SAV0904 | | 949224 | 951134 | 951040 | | |
| NMTN-08710 | SAV0904 | | 949224 | 951134 | 951136 | | |
| NMTN-08710 | SAV0905 | | 951134 | 952957 | 951136 | + | hypothetical protein |
| NMTN-15350 | SAV0905 | | 951134 | 952957 | 951173 | | |
| NMTN-00637 | SAV0905 | | 951134 | 952957 | 951287 | | |
| NMTN-13676 | SAV0905 | | 951134 | 952957 | 951506 | | |
| NMTN-08704 | SAV0905 | | 951134 | 952957 | 951905 | | |
| NMTN-12354 | SAV0905 | | 951134 | 952957 | 951967 | | |
| NMTN-12973 | SAV0905 | | 951134 | 952957 | 952148 | | |
| NMTN-14658 | SAV0905 | | 951134 | 952957 | 952244 | | |
| NMTN-11593 | SAV0905 | | 951134 | 952957 | 952310 | | |
| NMTN-12458 | SAV0905 | | 951134 | 952957 | 952310 | | |
| NMTN-03445 | SAV0905 | | 951134 | 952957 | 952371 | | |
| NMTN-01775 | SAV0905 | | 951134 | 952957 | 952411 | | |
| NMTN-14782 | SAV0905 | | 951134 | 952957 | 952418 | | |
| NMTN-09194 | SAV0905 | | 951134 | 952957 | 952484 | | |
| NMTN-07080 | SAV0906 | | 952957 | 953334 | 953038 | + | hypothetical protein |
| NMTN-11792 | SAV0908 | | 953552 | 953851 | 953550 | + | hypothetical protein |
| NMTN-06695 | SAV0908 | | 953552 | 953851 | 953763 | | |
| NMTN-11543 | SAV0908 | | 953552 | 953851 | 953774 | | |
| NMTN-04015 | SAV0909 | | 953988 | 955886 | 954056 | | |
| NMTN-06554 | SAV0909 | | 953988 | 955886 | 954074 | | hypothetical protein |
| NMTN-12488 | SAV0909 | | 953988 | 955886 | 954550 | | |
| NMTN-11630 | SAV0909 | | 953988 | 955886 | 954552 | | |
| NMTN-03088 | SAV0909 | | 953988 | 955886 | 954694 | | |
| NMTN-11535 | SAV0909 | | 953988 | 955886 | 954777 | | |
| NMTN-02487 | SAV0909 | | 953988 | 955886 | 954816 | | |
| NMTN-02254 | SAV0909 | | 953988 | 955886 | 954891 | | |
| NMTN-06371 | SAV0909 | | 953988 | 955886 | 955045 | | |
| NMTN-15334 | SAV0909 | | 953988 | 955886 | 955341 | | |
| NMTN-01519 | SAV0909 | | 953988 | 955886 | 955360 | | |
| NMTN-08047 | SAV0909 | | 953988 | 955886 | 955590 | | |
| NMTN-11325 | SAV0909 | | 953988 | 955886 | 955648 | | |
| NMTN-08591 | SAV0909 | | 953988 | 955886 | 955848 | | |
| NMTN-03217 | SAV0910 | | 955899 | 957137 | 955950 | + | hypothetical protein |
| NMTN-11143 | SAV0910 | | 955899 | 957137 | 955990 | | |
| NMTN-06328 | SAV0910 | | 955899 | 957137 | 955999 | | |
| NMTN-04887 | SAV0910 | | 955899 | 957137 | 956124 | | |
| NMTN-10617 | SAV0910 | | 955899 | 957137 | 956156 | | |
| NMTN-06522 | SAV0910 | | 955899 | 957137 | 956302 | | |
| NMTN-00672 | SAV0910 | | 955899 | 957137 | 956446 | | |
| NMTN-11311 | SAV0910 | | 955899 | 957137 | 956551 | | |
| NMTN-09475 | SAV0910 | | 955899 | 957137 | 956701 | | |
| NMTN-11165 | SAV0910 | | 955899 | 957137 | 956781 | | |
| NMTN-06135 | SAV0910 | | 955899 | 957137 | 957092 | | |
| NMTN-02505 | SAV0910 | | 955899 | 957137 | 957094 | | |
| NMTN-07062 | SAV0910 | | 955899 | 957137 | 957096 | | |
| NMTN-11362 | SAV0910 | | 955899 | 957137 | 957096 | | |
| NMTN-06173 | SAV0910 | | 955899 | 957137 | 957098 | | |
| NMTN-07722 | SAV0910 | | 955899 | 957137 | 957105 | | |
| NMTN-01580 | SAV0911 | | 957143 | 957538 | 957198 | + | hypothetical protein |
| NMTN-00455 | SAV0911 | | 957143 | 957538 | 957206 | | |
| NMTN-00487 | SAV0911 | | 957143 | 957538 | 957290 | | |
| NMTN-00559 | SAV0911 | | 957143 | 957538 | 957290 | | |
| NMTN-06996 | SAV0911 | | 957143 | 957538 | 957290 | | |
| NMTN-07058 | SAV0911 | | 957143 | 957538 | 957290 | | |
| NMTN-13651 | SAV0911 | | 957143 | 957538 | 957487 | | |
| NMTN-06605 | SAV0912 | | 957594 | 958031 | 957757 | + | hypothetical protein |
| NMTN-00479 | SAV0913 | | 958012 | 959457 | 958121 | + | amidase |
| NMTN-10896 | SAV0913 | | 958012 | 959457 | 958630 | | |
| NMTN-06570 | SAV0913 | | 958012 | 959457 | 959193 | | |
| NMTN-12527 | SAV0920 | | 964008 | 965075 | 964247 | + | hypothetical protein |
| NMTN-14315 | SAV0921 | | 965460 | 966308 | 965459 | + | conserved hypothetical protein |
| NMTN-04333 | SAV0921 | | 965460 | 966308 | 965514 | | |
| NMTN-00488 | SAV0921 | | 965460 | 966308 | 965743 | | |
| NMTN-06241 | SAV0921 | | 965460 | 966308 | 966264 | | |
| NMTN-06690 | SAV0922 | | 966321 | 967148 | 966450 | + | conserved hypothetical protein |
| NMTN-11244 | SAV0922 | | 966321 | 967148 | 966457 | | |
| NMTN-11604 | SAV0922 | | 966321 | 967148 | 966781 | | |
| NMTN-08672 | SAV0922 | | 966321 | 967148 | 966829 | | |
| NMTN-11612 | SAV0922 | | 966321 | 967148 | 966925 | | |
| NMTN-03068 | SAV0922 | | 966321 | 967148 | 967121 | | |
| NMTN-11050c | SAV0922 | | 966321 | 967148 | 967122 | | |
| NMTN-01629 | SAV0922/ | | 966321 | 967148 | 967122 | | |
| | SAV2176 | | | | | | |
| NMTN-06165 | SAV0922 | | 966321 | 967148 | 967127 | | |
| NMTN-11053c | SAV0922 | | 966321 | 967148 | 967137 | | |
| NMTN-11056c | SAV0922 | | 966321 | 967148 | 967145 | | |
| NMTN-09216 | SAV0923 | | 967175 | 968494 | 967220 | + | conserved hypothetical protein |
| NMTN-01811 | SAV0923 | | 967175 | 968494 | 967635 | | |
| NMTN-13174 | SAV0923 | | 967175 | 968494 | 967878 | | |
| NMTN-03980 | SAV0923 | | 967175 | 968494 | 967880 | | |
| NMTN-04926 | SAV0923 | | 967175 | 968494 | 968055 | | |
| NMTN-14624 | SAV0923 | | 967175 | 968494 | 968166 | | |
| NMTN-14703 | SAV0923 | | 967175 | 968494 | 968271 | | |
| NMTN-04030 | SAV0923 | | 967175 | 968494 | 968376 | | |
| NMTN-12149 | SAV0924 | lipA | 968578 | 969495 | 968810 | + | lipoic acid synthetase |
| NMTN-00703 | SAV0924 | lipA | 968578 | 969495 | 969268 | | |
| NMTN-12210 | SAV0924 | lipA | 968578 | 969495 | 969268 | | |
| NMTN-10729 | SAV0924 | lipA | 968578 | 969495 | 969308 | | |
| NMTN-02013 | SAV0924 | lipA | 968578 | 969495 | 969420 | | |
| NMTN-11001 | SAV0927 | | 971607 | 971873 | 971660 | − | conserved hypothetical protein |
| NMTN-00769 | SAV0927 | | 971607 | 971873 | 971685 | | |
| NMTN-05344 | SAV0928 | | 971971 | 972405 | 972012 | + | conserved hypothetical protein |
| NMTN-01968 | SAV0929 | | 972405 | 973184 | 972672 | + | hypothetical protein |
| NMTN-05388 | SAV0929 | | 972405 | 973184 | 972672 | | |
| NMTN-03519 | SAV0929 | | 972405 | 973184 | 973062 | | |
| NMTN-12314 | SAV0929 | | 972405 | 973184 | 973092 | | |
| NMTN-00653 | SAV0930 | | 973217 | 974176 | 973631 | + | hypothetical protein |
| NMTN-11315 | SAV0930 | | 973217 | 974176 | 973670 | | |
| NMTN-11221 | SAV0930 | | 973217 | 974176 | 973743 | | |
| NMTN-01709 | SAV0930 | | 973217 | 974176 | 974060 | | |
| NMTN-05246 | SAV0930 | | 973217 | 974176 | 974128 | | |
| NMTN-12076 | SAV0933 | dltB | 976210 | 977424 | 976498 | + | DltB membrane protein |
| NMTN-14377 | SAV0933 | dltB | 976210 | 977424 | 976940 | | |
| NMTN-09346 | SAV0935 | dltD | 977675 | 978850 | 977907 | + | poly (glycerophosphate chain) D-alanine |
| | | | | | | | transfer protein |
| NMTN-09243 | SAV0937 | | 979471 | 979779 | 979533 | | |
| NMTN-10387 | SAV0937 | | 979471 | 979779 | 979600 | + | conserved hypothetical protein |
| NMTN-04728 | SAV0937 | | 979471 | 979779 | 979603 | | |
| NMTN-12008 | SAV0937 | | 979471 | 979779 | 979724 | | |
| NMTN-00231 | SAV0938 | | 979838 | 980902 | 979857 | − | hypothetical protein |
| NMTN-07367 | SAV0938 | | 979838 | 980902 | 979990 | | |
| NMTN-12404 | SAV0938 | | 979838 | 980902 | 980396 | | |
| NMTN-12434 | SAV0938 | | 979838 | 980902 | 980455 | | |
| NMTN-13773 | SAV0938 | | 979838 | 980902 | 980541 | | |
| NMTN-10221 | SAV0938 | | 979838 | 980902 | 980629 | | |
| NMTN-10223 | SAV0938 | | 979838 | 980902 | 980629 | | |
| NMTN-10235 | SAV0938 | | 979838 | 980902 | 980629 | | |
| NMTN-13667 | SAV0938 | | 979838 | 980902 | 980665 | | |
| NMTN-09301 | SAV0939 | | 981219 | 981455 | 981207 | + | conserved hypothetical protein |
| NMTN-13891d | SAV0939 | | 981219 | 981455 | 981422 | | |
| NMTN-10876 | SAV0940 | | 981468 | 981827 | 981503 | + | conserved hypothetical protein |
| NMTN-01739 | SAV0941 | | 982281 | 983489 | 982281 | + | hypothetical protein |
| NMTN-05012 | SAV0942 | ampA | 983620 | 985095 | 984403 | + | probable cytosol aminopeptidase |
| NMTN-05015 | SAV0942 | ampA | 983620 | 985095 | 984403 | | |
| NMTN-08115 | SAV0942 | ampA | 983620 | 985095 | 984808 | | |
| NMTN-02308 | SAV0942 | ampA | 983620 | 985095 | 984911 | | |
| NMTN-11584 | SAV0943 | | 985506 | 986822 | 986523 | + | conserved hypothetical protein |
| NMTN-10576 | SAV0944 | | 986841 | 987215 | 986853 | + | conserved hypothetical protein |
| NMTN-05437 | SAV0944 | | 986841 | 987215 | 986926 | | |
| NMTN-04008 | SAV0945 | | 987271 | 988425 | 987690 | − | hypothetical protein |
| NMTN-08972 | SAV0945 | | 987271 | 988425 | 988239 | | |
| NMTN-07555 | SAV0956 | | 996763 | 997890 | 996759 | + | hypothetical protein |
| NMTN-07359 | SAV0956 | | 996763 | 997890 | 996841 | | |
| NMTN-09242 | SAV0956 | | 996763 | 997890 | 997300 | | |
| NMTN-12501 | SAV0956 | | 996763 | 997890 | 997432 | | |
| NMTN-11078 | SAV0956 | | 996763 | 997890 | 997678 | | |
| NMTN-12260 | SAV0956 | | 996763 | 997890 | 997886 | | |
| NMTN-03954 | SAV0957 | rocD | 998198 | 999388 | 998589 | + | ornithine aminotransferase |
| NMTN-11547 | SAV0957 | rocD | 998198 | 999388 | 998726 | | |
| NMTN-07736 | SAV0958 | gudB | 999497 | 1000741 | 999884 | + | NAD-specific glutamate dehydrogenase |
| NMTN-14019 | SAV0958 | gudB | 999497 | 1000741 | 1000080 | | |
| NMTN-02830 | SAV0958 | gudB | 999497 | 1000741 | 1000428 | | |
| NMTN-10720 | SAV0958 | gudB | 999497 | 1000741 | 1000449 | | |
| NMTN-00026 | SAV0959 | glpQ | 1001078 | 1002007 | 1001197 | − | glycerophosphoryl diester |
| | | | | | | | phosphodiesterase |
| NMTN-00806 | SAV0959 | glpQ | 1001078 | 1002007 | 1001706 | | |
| NMTN-11755 | SAV0960 | argH | 1002249 | 1003628 | 1002446 | − | argininosuccinate lyase |
| NMTN-03785 | SAV0960 | argH | 1002249 | 1003628 | 1002716 | | |
| NMTN-13726 | SAV0960 | argH | 1002249 | 1003628 | 1002745 | | |
| NMTN-04599 | SAV0960 | argH | 1002249 | 1003628 | 1002927 | | |
| NMTN-08885 | SAV0962 | pgi | 1005174 | 1006505 | 1006452 | + | glucose-6-phosphate isomerase A |
| NMTN-00327 | SAV0963 | | 1006830 | 1007405 | 1007280 | + | conserved hypothetical protein |
| NMTN-02078 | SAV0968 | | 1015981 | 1016883 | 1016366 | + | hypothetical protein |
| NMTN-08749 | SAV0968 | | 1015981 | 1016883 | 1016410 | | |
| NMTN-09319 | SAV0968 | | 1015981 | 1016883 | 1016871 | | |
| NMTN-02923 | SAV0969 | | 1017212 | 1017601 | 1017218 | + | conserved hypothetical protein |
| NMTN-08231 | SAV0969 | | 1017212 | 1017601 | 1017226 | | |
| NMTN-09429 | SAV0969 | | 1017212 | 1017601 | 1017227 | | |
| NMTN-05804 | SAV0969 | | 1017212 | 1017601 | 1017228 | | |
| NMTN-10802 | SAV0969 | | 1017212 | 1017601 | 1017420 | | |
| NMTN-09957 | SAV0969 | | 1017212 | 1017601 | 1017422 | | |
| NMTN-08996 | SAV0969 | | 1017212 | 1017601 | 1017548 | | |
| NMTN-09924 | SAV0969 | | 1017212 | 1017601 | 1017550 | | |
| NMTN-04039 | SAV0969 | | 1017212 | 1017601 | 1017551 | | |
| NMTN-06962 | SAV0970 | cdr | 1017773 | 1019089 | 1018098 | − | coenzyme A disulfide reductase |
| NMTN-03101 | SAV0970 | cdr | 1017773 | 1019089 | 1018179 | | |
| NMTN-11697 | SAV0970 | cdr | 1017773 | 1019089 | 1018392 | | |
| NMTN-12928 | SAV0974 | | 1020935 | 1022749 | 1020976 | + | hypothetical protein |
| NMTN-06647 | SAV0974 | | 1020935 | 1022749 | 1021193 | | |
| NMTN-05620 | SAV0974 | | 1020935 | 1022749 | 1021202 | | |
| NMTN-02941 | SAV0974 | | 1020935 | 1022749 | 1021620 | | |
| NMTN-06916 | SAV0974 | | 1020935 | 1022749 | 1021649 | | |
| NMTN-00524 | SAV0974 | | 1020935 | 1022749 | 1021670 | | |
| NMTN-11337 | SAV0974 | | 1020935 | 1022749 | 1021901 | | |
| NMTN-11403 | SAV0974 | | 1020935 | 1022749 | 1021915 | | |
| NMTN-02991 | SAV0974 | | 1020935 | 1022749 | 1021936 | | |
| NMTN-12060 | SAV0974 | | 1020935 | 1022749 | 1021939 | | |
| NMTN-11662 | SAV0974 | | 1020935 | 1022749 | 1022544 | | |
| NMTN-07833 | SAV0975 | clpB | 1022952 | 1025561 | 1023086 | + | ClpB chaperone homologue |
| NMTN-08275 | SAV0975 | clpB | 1022952 | 1025561 | 1023099 | | |
| NMTN-14455 | SAV0975 | clpB | 1022952 | 1025561 | 1024434 | | |
| NMTN-05214 | SAV0976 | | 1025620 | 1026489 | 1025701 | − | hypothetical protein |
| NMTN-03545 | SAV0976 | | 1025620 | 1026489 | 1025777 | | |
| NMTN-06935 | SAV0976 | | 1025620 | 1026489 | 1025844 | | |
| NMTN-06518 | SAV0976 | | 1025620 | 1026489 | 1025978 | | |
| NMTN-11614 | SAV0976 | | 1025620 | 1026489 | 1026398 | | |
| NMTN-00694 | SAV0977 | | 1026599 | 1027744 | 1026902 | + | hypothetical protein |
| NMTN-12509 | SAV0977 | | 1026599 | 1027744 | 1027045 | + | conserved hypothetical protein |
| NMTN-06247 | SAV0978 | | 1027728 | 1028369 | 1028056 | | |
| NMTN-05299 | SAV0978 | | 1027728 | 1028369 | 1028149 | | |
| NMTN-02143 | SAV0978 | | 1027728 | 1028369 | 1028222 | | |
| NMTN-12884 | SAV0979 | | 1028362 | 1029483 | 1028460 | + | hypothetical protein |
| NMTN-05409 | SAV0979 | | 1028362 | 1029483 | 1028526 | | |
| NMTN-14611 | SAV0979 | | 1028362 | 1029483 | 1028614 | | |
| NMTN-07415 | SAV0979 | | 1028362 | 1029483 | 1028791 | | |
| NMTN-09008 | SAV0979 | | 1028362 | 1029483 | 1029179 | | |
| NMTN-12613 | SAV0979 | | 1028362 | 1029483 | 1029182 | | |
| NMTN-03567 | SAV0979 | | 1028362 | 1029483 | 1029185 | | |
| NMTN-05568 | SAV0979 | | 1028362 | 1029483 | 1029305 | | |
| NMTN-11791 | SAV0979 | | 1028362 | 1029483 | 1029350 | | |
| NMTN-01532 | SAV0981 | | 1030287 | 1030721 | 1030506 | + | hypothetical protein |
| NMTN-04633 | SAV0982 | | 1030978 | 1031163 | 1031099 | − | hypothetical protein |
| NMTN-04047 | SAV0985 | | 1033710 | 1034081 | 1033786 | − | hypothetical protein |
| NMTN-13898 | SAV0986 | oppB | 1034324 | 1035250 | 1034398 | + | oligopeptide transport system permease |
| | | | | | | | protein |
| NMTN-02880 | SAV0986 | oppB | 1034324 | 1035250 | 1034988 | | |
| NMTN-08460 | SAV0987 | | 1035250 | 1036320 | 1035559 | + | hypothetical protein |
| NMTN-02280 | SAV0987 | | 1035250 | 1036320 | 1036180 | | |
| NMTN-06450 | SAV0987 | | 1035250 | 1036320 | 1036261 | | |
| NMTN-14304 | SAV0988 | oppD | 1036336 | 1037418 | 1037092 | + | oligopeptide transport system ATP- |
| | | | | | | | binding protein OppD homologue |
| NMTN-08680 | SAV0989 | oppF | 1037408 | 1038349 | 1037633 | + | oligopeptide transport system ATP- |
| | | | | | | | binding protein OppF homologue |
| NMTN-04084 | SAV0989 | oppF | 1037408 | 1038349 | 1037806 | | |
| NMTN-11394 | SAV0989 | oppF | 1037408 | 1038349 | 1038263 | | |
| NMTN-10397 | SAV0990 | | 1038368 | 1040023 | 1038541 | + | hypothetical protein |
| NMTN-10486 | SAV0990 | | 1038368 | 1040023 | 1038654 | | |
| NMTN-02040 | SAV0990 | | 1038368 | 1040023 | 1038798 | | |
| NMTN-08760 | SAV0990 | | 1038368 | 1040023 | 1039040 | | |
| NMTN-09596 | SAV0990 | | 1038368 | 1040023 | 1039135 | | |
| NMTN-06703 | SAV0990 | | 1038368 | 1040023 | 1039910 | | |
| NMTN-12419 | SAV0990 | | 1038368 | 1040023 | 1039969 | | |
| NMTN-11494 | SAV0990 | | 1038368 | 1040023 | 1039976 | | |
| NMTN-00303 | SAV0991 | | 1040235 | 1041950 | 1040382 | + | hypothetical protein |
| NMTN-03536 | SAV0991 | | 1040235 | 1041950 | 1040428 | | |
| NMTN-10854 | SAV0991 | | 1040235 | 1041950 | 1041368 | | |
| NMTN-06577 | SAV0992 | | 1042001 | 1042987 | 1042242 | + | oligopeptide ABC transporter ATP- |
| | | | | | | | binding protein homologue |
| NMTN-01951 | SAV0992 | | 1042001 | 1042987 | 1042753 | | |
| NMTN-05621 | SAV0993 | appF | 1042990 | 1043970 | 1043347 | + | oligopeptide transport system ATP- |
| | | | | | | | binding protein AppF homologue |
| NMTN-03178 | SAV0993 | appF | 1042990 | 1043970 | 1043416 | | |
| NMTN-04918 | SAV0993 | appF | 1042990 | 1043970 | 1043435 | | |
| NMTN-05606 | SAV0993 | appF | 1042990 | 1043970 | 1043530 | | |
| NMTN-13365d | SAV0993 | appF | 1042990 | 1043970 | 1043614 | | |
| NMTN-04720 | SAV0993 | appF | 1042990 | 1043970 | 1043638 | | |
| NMTN-00272 | SAV0993 | appF | 1042990 | 1043970 | 1043650 | | |
| NMTN-03960 | SAV0993 | appF | 1042990 | 1043970 | 1043686 | | |
| NMTN-01992 | SAV0993 | appF | 1042990 | 1043970 | 1043777 | | |
| NMTN-06455 | SAV0994 | oppB | 1043963 | 1044925 | 1043982 | + | probable oligopeptide transport system |
| | | | | | | | permease protein |
| NMTN-13513 | SAV0994 | oppB | 1043963 | 1044925 | 1044185 | | |
| NMTN-14170 | SAV0994 | oppB | 1043963 | 1044925 | 1044185 | | |
| NMTN-03876 | SAV0994 | oppB | 1043963 | 1044925 | 1044292 | | |
| NMTN-14776 | SAV0994 | oppB | 1043963 | 1044925 | 1044373 | | |
| NMTN-03912 | SAV0994 | oppB | 1043963 | 1044925 | 1044514 | | |
| NMTN-08776 | SAV0994 | oppB | 1043963 | 1044925 | 1044800 | | |
| NMTN-10014 | SAV0994 | oppB | 1043963 | 1044925 | 1044893 | | |
| NMTN-10629 | SAV0995 | | 1044937 | 1045818 | 1045409 | + | hypothetical protein |
| NMTN-11596 | SAV0995 | | 1044937 | 1045818 | 1045462 | | |
| NMTN-05436 | SAV0998 | | 1047909 | 1408628 | 1047961 | + | hypothetical protein |
| NMTN-00680 | SAV0998 | | 1047909 | 1408628 | 1048424 | | |
| NMTN-03907 | SAV1000 | | 1049783 | 1051591 | 1049781 | + | thimet oligopeptidase homologue |
| NMTN-04804 | SAV1000 | | 1049783 | 1051591 | 1049817 | | |
| NMTN-13731 | SAV1000 | | 1049783 | 1051591 | 1050013 | | |
| NMTN-11452 | SAV1000 | | 1049783 | 1051591 | 1050400 | | |
| NMTN-11270 | SAV1000 | | 1049783 | 1051591 | 1050462 | | |
| NMTN-12448 | SAV1000 | | 1049783 | 1051591 | 1050689 | | |
| NMTN-08998 | SAV1000 | | 1049783 | 1051591 | 1050723 | | |
| NMTN-13237 | SAV1000 | | 1049783 | 1051591 | 1050924 | | |
| NMTN-00315 | SAV1000 | | 1049783 | 1051591 | 1050944 | | |
| NMTN-05368 | SAV1001 | yjbM | 1052051 | 1052605 | 1052187 | − | GTP pyrophosphokinase |
| NMTN-06682 | SAV1002 | | 1052541 | 1052867 | 1052612 | − | conserved hypothetical protein |
| NMTN-07348 | SAV1004 | | 1053353 | 1053946 | 1053766 | − | conserved hypothetical protein |
| NMTN-02696 | SAV1004 | | 1053353 | 1053946 | 1053824 | | |
| NMTN-11970 | SAV1004 | | 1053353 | 1053946 | 1053955 | | |
| NMTN-04841 | SAV1008 | | 1055954 | 1056808 | 1056345 | + | conserved hypothetical protein |
| NMTN-06475 | SAV1008 | | 1055954 | 1056808 | 1056609 | | |
| NMTN-11935 | SAV1009 | | 1056829 | 1058214 | 1057061 | + | hypothetical protein |
| NMTN-04961 | SAV1009 | | 1056829 | 1058214 | 1057110 | | |
| NMTN-12106 | SAV1009 | | 1056829 | 1058214 | 1057284 | | |
| NMTN-11226 | SAV1009 | | 1056829 | 1058214 | 1057441 | | |
| NMTN-11218 | SAV1009 | | 1056829 | 1058214 | 1057451 | | |
| NMTN-02703 | SAV1009 | | 1056829 | 1058214 | 1058165 | | |
| NMTN-10401 | SAV1010 | | 1058224 | 1060068 | 1058362 | + | Na+/H+ antiporter homologue |
| NMTN-00142 | SAV1010 | | 1058224 | 1060068 | 1058581 | | |
| NMTN-12609 | SAV1010 | | 1058224 | 1060068 | 1058810 | | |
| NMTN-15202 | SAV1010 | | 1058224 | 1060068 | 1058847 | | |
| NMTN-01921 | SAV1010 | | 1058224 | 1060068 | 1059508 | | |
| NMTN-13330 | SAV1010 | | 1058224 | 1060068 | 1059881 | | |
| NMTN-10818 | SAV1010 | | 1058224 | 1060068 | 1059882 | | |
| NMTN-14708 | SAV1012 | | 1061313 | 1062398 | 1061585 | − | conserved hypothetical protein |
| NMTN-14573 | SAV1012 | | 1061313 | 1062398 | 1061618 | | |
| NMTN-09644 | SAV1012 | | 1061313 | 1062398 | 1061637 | | |
| NMTN-00126 | SAV1012 | | 1061313 | 1062398 | 1061710 | | |
| NMTN-07981 | SAV1012 | | 1061313 | 1062398 | 1061842 | | |
| NMTN-13832 | SAV1013 | | 1062803 | 1064308 | 1062802 | + | hypothetical protein |
| NMTN-11732 | SAV1013 | | 1062803 | 1064308 | 1063340 | | |
| NMTN-02794 | SAV1013 | | 1062803 | 1064308 | 1063387 | | |
| NMTN-15262 | SAV1013 | | 1062803 | 1064308 | 1063696 | | |
| NMTN-14646 | SAV1013 | | 1062803 | 1064308 | 1063810 | | |
| NMTN-10663 | SAV1013 | | 1062803 | 1064308 | 1064148 | | |
| NMTN-08774 | SAV1014 | | 1064453 | 1065211 | 1064563 | + | conserved hypothetical protein |
| NMTN-08458 | SAV1014 | | 1064453 | 1065211 | 1065088 | | |
| NMTN-06998 | SAV1019 | | 1070277 | 1070528 | 1070510 | + | hypothetical protein |
| NMTN-11246 | SAV1019 | | 1070277 | 1070528 | 1070537 | | |
| NMTN-04654 | SAV1021 | | 1070918 | 1072135 | 1070988 | + | peptide chain release factor 3 |
| NMTN-13507 | SAV1021 | | 1070918 | 1072135 | 1071005 | | |
| NMTN-12678 | SAV1021 | | 1070918 | 1072135 | 1071655 | | |
| NMTN-12188 | SAV1021 | | 1070918 | 1072135 | 1071656 | | |
| NMTN-08059 | SAV1021 | | 1070918 | 1072135 | 1071714 | | |
| NMTN-06388 | SAV1021 | | 1070918 | 1072135 | 1071838 | | |
| NMTN-12561 | SAV1023 | htrA | 1073474 | 1075783 | 1074247 | + | serine protease HtrA |
| NMTN-13714 | SAV1023 | htrA | 1073474 | 1075783 | 1074690 | | |
| NMTN-09232 | SAV1023 | htrA | 1073474 | 1075783 | 1074783 | | |
| NMTN-04400 | SAV1023 | htrA | 1073474 | 1075783 | 1074999 | | |
| NMTN-09181 | SAV1024 | | 1075800 | 1077158 | 1075859 | + | hypothetical protein |
| NMTN-13301 | SAV1024 | | 1075800 | 1077158 | 1075862 | | |
| NMTN-10850 | SAV1024 | | 1075800 | 1077158 | 1076319 | | |
| NMTN-07776 | SAV1024 | | 1075800 | 1077158 | 1076464 | | |
| NMTN-02935 | SAV1024 | | 1075800 | 1077158 | 1076878 | | |
| NMTN-10003 | SAV1024 | | 1075800 | 1077158 | 1077128 | | |
| NMTN-14045 | SAV1025 | | 1077294 | 1078808 | 1077349 | + | hypothetical protein |
| NMTN-07756 | SAV1025 | | 1077294 | 1078808 | 1077821 | | |
| NMTN-05509 | SAV1025 | | 1077294 | 1078808 | 1078181 | | |
| NMTN-12367 | SAV1025 | | 1077294 | 1078808 | 1078334 | | |
| NMTN-11551 | SAV1025 | | 1077294 | 1078808 | 1078335 | | |
| NMTN-07277 | SAV1025 | | 1077294 | 1078808 | 1078468 | | |
| NMTN-05273 | SAV1025 | | 1077294 | 1078808 | 1078471 | | |
| NMTN-07536 | SAV1025 | | 1077294 | 1078808 | 1078510 | | |
| NMTN-10599 | SAV1025 | | 1077294 | 1078808 | 1078657 | | |
| NMTN-15349 | SAV1025 | | 1077294 | 1078808 | 1078718 | | |
| NMTN-03801 | SAV1028 | | 1080372 | 1081358 | 1080677 | − | lipoate-protein ligase homolog |
| NMTN-11919 | SAV1028 | | 1080372 | 1081358 | 1080742 | | |
| NMTN-10696 | SAV1029 | | 1081557 | 1081733 | 1081552 | + | conserved hypothetical protein |
| NMTN-00658 | SAV1028 | | 1080372 | 1081358 | 1081555 | | |
| NMTN-13251 | SAV1030 | | 1081748 | 1082350 | 1081818 | + | hypothetical protein |
| NMTN-04348 | SAV1030 | | 1081748 | 1082350 | 1081853 | | |
| NMTN-06826 | SAV1030 | | 1081748 | 1082350 | 1081863 | | |
| NMTN-14485 | SAV1030 | | 1081748 | 1082350 | 1081967 | | |
| NMTN-02390 | SAV1030 | | 1081748 | 1082350 | 1082193 | | |
| NMTN-12523 | SAV1030 | | 1081748 | 1082350 | 1082291 | | |
| NMTN-02382 | SAV1030 | | 1081748 | 1082350 | 1082324 | | |
| NMTN-07020 | SAV1030 | | 1081748 | 1082350 | 1082327 | | |
| NMTN-05080 | SAV1031 | | 1082559 | 1082720 | 1082588 | + | hypothetical protein |
| NMTN-02673 | SAV1032 | | 1083377 | 1083649 | 1083634 | + | hypothetical protein |
| NMTN-00554 | SAV1033 | | 1083693 | 1085657 | 1083753 | + | hypothetical protein |
| NMTN-14601 | SAV1033 | | 1083693 | 1085657 | 1083760 | | |
| NMTN-11250 | SAV1033 | | 1083693 | 1085657 | 1083942 | | |
| NMTN-02898 | SAV1033 | | 1083693 | 1085657 | 1083943 | | |
| NMTN-04931 | SAV1033 | | 1083693 | 1085657 | 1083943 | | |
| NMTN-12762 | SAV1033 | | 1083693 | 1085657 | 1084131 | | |
| NMTN-09504 | SAV1033 | | 1083693 | 1085657 | 1084389 | | |
| NMTN-10255 | SAV1033 | | 1083693 | 1085657 | 1084518 | | |
| NMTN-13829 | SAV1033 | | 1083693 | 1085657 | 1085122 | | |
| NMTN-01790 | SAV1033 | | 1083693 | 1085657 | 1085125 | | |
| NMTN-13563 | SAV1033 | | 1083693 | 1085657 | 1085309 | | |
| NMTN-08413 | SAV1033 | | 1083693 | 1085657 | 1085400 | | |
| NMTN-05363 | SAV1033 | | 1083693 | 1085657 | 1085499 | | |
| NMTN-06660 | SAV1033 | | 1083693 | 1085657 | 1085500 | | |
| NMTN-13234 | SAV1034 | | 1085660 | 1085980 | 1085945 | + | conserved hypothetical protein |
| NMTN-03633 | SAV1035 | | 1085977 | 1086618 | 1086113 | + | conserved hypothetical protein |
| NMTN-13696 | SAV1037 | | 1087363 | 1087719 | 1087706 | − | conserved hypothetical protein |
| NMTN-13696 | SAV1037 | | 1087363 | 1087719 | 1087706 | | |
| NMTN-07906 | SAV1038 | | 1088207 | 1089166 | 1088926 | + | hypothetical protein |
| NMTN-00261 | SAV1039 | | 1089407 | 1089622 | 1089571 | − | hypothetical protein |
| NMTN-03175 | SAV1039 | | 1089407 | 1089622 | 1089612 | | |
| NMTN-05328 | SAV1040 | | 1089787 | 1090338 | 1089982 | + | conserved hypothetical protein |
| NMTN-12607 | SAV1040 | | 1089787 | 1090338 | 1090002 | | |
| NMTN-03688 | SAV1040 | | 1089787 | 1090338 | 1090205 | | |
| NMTN-11267 | SAV1043 | menD | 1092858 | 1094531 | 1093227 | + | menaquinone biosynthesis protein |
| NMTN-08028 | SAV1044 | | 1094518 | 1095321 | 1094882 | + | hypothetical protein |
| NMTN-09687 | SAV1044 | | 1094518 | 1095321 | 1095210 | | |
| NMTN-07533 | SAV1044 | | 1094518 | 1095321 | 1095216 | | |
| NMTN-12636 | SAV1046 | sspC | 1096373 | 1096702 | 1096475 | − | cysteine protease |
| NMTN-04732 | SAV1046 | sspC | 1096373 | 1096702 | 1096648 | | |
| NMTN-01572 | SAV1047 | sspB | 1096740 | 1097921 | 1096796 | − | cysteine protease precursor |
| NMTN-10512 | SAV1047 | sspB | 1096740 | 1097921 | 1097641 | | |
| NMTN-13070 | SAV1048 | sspA | 1098003 | 1099031 | 1098494 | − | serine protease |
| NMTN-03465 | SAV1049 | | 1099559 | 1100713 | 1099837 | − | HisC homolog |
| NMTN-09873 | SAV1049 | | 1099559 | 1100713 | 1099935 | | |
| NMTN-12250 | SAV1049 | | 1099559 | 1100713 | 1100562 | | |
| NMTN-13484 | SAV1050 | | 1100909 | 1101919 | 1101113 | − | conserved hypothetical protein |
| NMTN-13489 | SAV1050 | | 1100909 | 1101919 | 1101113 | | |
| NMTN-02963 | SAV1050 | | 1100909 | 1101919 | 1101137 | | |
| NMTN-08146 | SAV1050 | | 1100909 | 1101919 | 1101216 | | |
| NMTN-12951 | SAV1050 | | 1100909 | 1101919 | 1101384 | | |
| NMTN-02744 | SAV1050 | | 1100909 | 1101919 | 1101716 | | |
| NMTN-12102 | SAV1052 | truncated- | 1102698 | 1103504 | 1102803 | − | partial autolysin |
| | | atl | | | | | |
| NMTN-12110 | SAV1052 | truncated- | 1102698 | 1103504 | 1102803 | | |
| | | atl | | | | | |
| NMTN-12112 | SAV1052 | truncated- | 1102698 | 1103504 | 1102803 | | |
| | | atl | | | | | |
| NMTN-12121 | SAV1052 | truncated- | 1102698 | 1103504 | 1102803 | | |
| | | atl | | | | | |
| NMTN-12103 | SAV1052 | truncated- | 1102698 | 1103504 | 1102805 | | |
| | | atl | | | | | |
| NMTN-07710 | SAV1052 | truncated- | 1102698 | 1103504 | 1102903 | | |
| | | atl | | | | | |
| NMTN-06238 | SAV1052 | truncated- | 1102698 | 1103504 | 1103419 | | |
| | | atl | | | | | |
| NMTN-03732 | SAV1053 | atl | 1103624 | 1106455 | 1104246 | − | autolysin |
| NMTN-11530 | SAV1053 | atl | 1103624 | 1106455 | 1104698 | | |
| NMTN-10038 | SAV1053 | atl | 1103624 | 1106455 | 1105626 | | |
| NMTN-04043 | SAV1053 | atl | 1103624 | 1106455 | 1105876 | | |
| NMTN-04970 | SAV1053 | atl | 1103624 | 1106455 | 1106091 | | |
| NMTN-06233 | SAV1054 | | 1106662 | 1107096 | 1106723 | − | conserved hypothetical protein |
| NMTN-10201 | SAV1054 | | 1106662 | 1107096 | 1107031 | | |
| NMTN-10442 | SAV1057 | fmt | 1109437 | 1110630 | 1109757 | + | autolysis and methicillin resistant-related |
| | | | | | | | protein |
| NMTN-06459 | SAV1057 | fmt | 1109437 | 1110630 | 1109972 | | |
| NMTN-13704 | SAV1057 | fmt | 1109437 | 1110630 | 1110340 | | |
| NMTN-05261 | SAV1057 | fmt | 1109437 | 1110630 | 1110435 | | |
| NMTN-02055 | SAV1060 | qoxB | 1112049 | 1114037 | 1112432 | − | Quinol oxidase polypeptide I |
| NMTN-02302 | SAV1060 | qoxB | 1112049 | 1114037 | 1112818 | | |
| NMTN-00741 | SAV1062 | | 1115708 | 1116025 | 1115721 | − | hypothetical protein |
| NMTN-05543 | SAV1062 | | 1115708 | 1116025 | 1115726 | | |
| NMTN-02380 | SAV1064 | | 1117950 | 1118432 | 1118021 | + | hypothetical protein |
| NMTN-14653 | SAV1064 | | 1117950 | 1118432 | 1118164 | | |
| NMTN-05091 | SAV1065 | purK | 1118419 | 1119543 | 1118592 | + | phosphoribosylaminoimidazole |
| | | | | | | | carboxylase carbon dioxide-fixation |
| | | | | | | | chain Purk homolog |
| NMTN-04806 | SAV1065 | purK | 1118419 | 1119543 | 1118948 | | |
| NMTN-02006 | SAV1065 | purK | 1118419 | 1119543 | 1119008 | | |
| NMTN-12746 | SAV1065 | purK | 1118419 | 1119543 | 1119425 | | |
| NMTN-02393 | SAV1065 | purK | 1118419 | 1119543 | 1119496 | | |
| NMTN-03769 | SAV1065 | purK | 1118419 | 1119543 | 1119515 | | |
| NMTN-02924 | SAV1066 | purC | 1119547 | 1120251 | 1119556 | + | phosphoribosylamino- |
| | | | | | | | imidazolesuccinocarboxamide |
| | | | | | | | synthetase homolog |
| NMTN-06530 | SAV1066 | purC | 1119547 | 1120251 | 1119556 | | |
| NMTN-01626 | SAV1066 | purC | 1119547 | 1120251 | 1119707 | | |
| NMTN-08758 | SAV1066 | purC | 1119547 | 1120251 | 1120043 | | |
| NMTN-01946 | SAV1066 | purC | 1119547 | 1120251 | 1120127 | | |
| NMTN-07718 | SAV1066 | purC | 1119547 | 1120251 | 1120180 | | |
| NMTN-02602 | SAV1066 | purC | 1119547 | 1120251 | 1120181 | | |
| NMTN-12894 | SAV1067 | | 1120251 | 1120514 | 1120335 | + | conserved hypothetical protein |
| NMTN-05399 | SAV1068 | purQ | 1120516 | 1121187 | 1120616 | + | phosphoribosylformylglycinamidine |
| | | | | | | | synthase I |
| NMTN-06147 | SAV1068 | purQ | 1120516 | 1121187 | 1121074 | | |
| NMTN-02002 | SAV1069 | purL | 1121180 | 1123369 | 1122317 | + | phosphoribosylformylglycinamidine |
| | | | | | | | synthetase |
| NMTN-08158 | SAV1069 | purL | 1121180 | 1123369 | 1122385 | | |
| NMTN-05314 | SAV1069 | purL | 1121180 | 1123369 | 1122420 | | |
| NMTN-10940 | SAV1069 | purL | 1121180 | 1123369 | 1122429 | | |
| NMTN-07153 | SAV1069 | purL | 1121180 | 1123369 | 1122782 | | |
| NMTN-03227 | SAV1069 | purL | 1121180 | 1123369 | 1123314 | | |
| NMTN-09570 | SAV1069 | purL | 1121180 | 1123369 | 1123328 | | |
| NMTN-04992 | SAV1070 | purF | 1123348 | 1124832 | 1123425 | + | phosphoribosylpyrophosphate |
| | | | | | | | amidotransferase |
| NMTN-12566 | SAV1070 | purF | 1123348 | 1124832 | 1123425 | | |
| NMTN-03038 | SAV1070 | purF | 1123348 | 1124832 | 1123428 | | |
| NMTN-13190 | SAV1070 | purF | 1123348 | 1124832 | 1124030 | | |
| NMTN-09228 | SAV1070 | purF | 1123348 | 1124832 | 1124063 | | |
| NMTN-05294 | SAV1070 | purF | 1123348 | 1124832 | 1124102 | | |
| NMTN-03419 | SAV1070 | purF | 1123348 | 1124832 | 1124321 | | |
| NMTN-10821 | SAV1070 | purF | 1123348 | 1124832 | 1124641 | | |
| NMTN-00869 | SAV1070 | purF | 1123348 | 1124832 | 1124676 | | |
| NMTN-06381 | SAV1071 | purM | 1124825 | 1125853 | 1125171 | + | phosphoribosylformylglycinamidine |
| | | | | | | | cyclo-ligase |
| NMTN-03863 | SAV1071 | purM | 1124825 | 1125853 | 1125468 | | |
| NMTN-07785 | SAV1072 | purN | 1125856 | 1126422 | 1126030 | + | phosphoribosylglycinamide |
| | | | | | | | formyltransferase |
| NMTN-07721 | SAV1072 | purN | 1125856 | 1126422 | 1126066 | | |
| NMTN-12649 | SAV1073 | purH | 1126434 | 1127915 | 1126431 | + | bifunctional purine biosynthesis protein |
| NMTN-04103 | SAV1073 | purH | 1126434 | 1127915 | 1126528 | | |
| NMTN-10455 | SAV1073 | purH | 1126434 | 1127915 | 1126546 | | |
| NMTN-11708 | SAV1073 | purH | 1126434 | 1127915 | 1126710 | | |
| NMTN-02356 | SAV1073 | purH | 1126434 | 1127915 | 1126902 | | |
| NMTN-02573 | SAV1073 | purH | 1126434 | 1127915 | 1126930 | | |
| NMTN-02443 | SAV1073 | purH | 1126434 | 1127915 | 1127238 | | |
| NMTN-02394 | SAV1073 | purH | 1126434 | 1127915 | 1127394 | | |
| NMTN-10721 | SAV1074 | purD | 1128045 | 1129184 | 1128409 | + | phosphoribosylamine-glycine ligase |
| NMTN-09084 | SAV1074 | purD | 1128045 | 1129184 | 1128571 | | |
| NMTN-03896 | SAV1074 | purD | 1128045 | 1129184 | 1128966 | | |
| NMTN-06507 | SAV1074 | purD | 1128045 | 1129184 | 1128966 | | |
| NMTN-11668 | SAV1075 | | 1129452 | 1130258 | 1129567 | − | conserved hypothetical protein |
| NMTN-12839 | SAV1075 | | 1129452 | 1130258 | 1129682 | | |
| NMTN-12178 | SAV1075 | | 1129452 | 1130258 | 1129685 | | |
| NMTN-03789 | SAV1075 | | 1129452 | 1130258 | 1130162 | | |
| NMTN-06360 | SAV1075 | | 1129452 | 1130258 | 1130209 | | |
| NMTN-11127 | SAV1076 | | 1130251 | 1131651 | 1130672 | − | hypothetical protein |
| NMTN-12840 | SAV1076 | | 1130251 | 1131651 | 1130992 | | |
| NMTN-05649 | SAV1076 | | 1130251 | 1131651 | 1131043 | | |
| NMTN-13746 | SAV1077 | | 1131666 | 1132241 | 1131846 | − | conserved hypothetical protein |
| NMTN-07237 | SAV1081 | | 1135082 | 1136254 | 1135421 | + | conserved hypothetical protein |
| NMTN-12731 | SAV1081 | | 1135082 | 1136254 | 1135935 | | |
| NMTN-00570 | SAV1081 | | 1135082 | 1136254 | 1136066 | | |
| NMTN-03266 | SAV1081 | | 1135082 | 1136254 | 1136089 | | |
| NMTN-09129 | SAV1081 | | 1135082 | 1136254 | 1136115 | | |
| NMTN-00870 | SAV1084 | ptsI | 1137273 | 1138991 | 1137354 | + | phosphoenolpyruvate-protein |
| | | | | | | | phosphatase |
| NMTN-13743 | SAV1086 | | 1139659 | 1141020 | 1139801 | + | cytochrome D ubiquinol oxidase subunit |
| | | | | | | | 1 homolog |
| NMTN-07264 | SAV1086 | | 1139659 | 1141020 | 1140004 | | |
| NMTN-14450 | SAV1086 | | 1139659 | 1141020 | 1140055 | | |
| NMTN-06439 | SAV1086 | | 1139659 | 1141020 | 1140161 | | |
| NMTN-14445d | SAV1086 | | 1139659 | 1141020 | 1140548 | | |
| NMTN-10388 | SAV1086 | | 1139659 | 1141020 | 1140997 | | |
| NMTN-07732d | SAV1086 | | 1139659 | 1141020 | 1141027 | | |
| NMTN-07732d | SAV1087 | | 1141017 | 1142036 | 1141027 | + | cytochrome D ubiquinol oxidase subunit |
| | | | | | | | II homolog |
| NMTN-08761 | SAV1087 | | 1141017 | 1142036 | 1141414 | | |
| NMTN-13858 | SAV1087 | | 1141017 | 1142036 | 1141422 | | |
| NMTN-13642 | SAV1087 | | 1141017 | 1142036 | 1141426 | | |
| NMTN-02588 | SAV1087 | | 1141017 | 1142036 | 1141444 | | |
| NMTN-10071 | SAV1087 | | 1141017 | 1142036 | 1141665 | | |
| NMTN-09898 | SAV1087 | | 1141017 | 1142036 | 1141671 | | |
| NMTN-07272 | SAV1087 | | 1141017 | 1142036 | 1141901 | | |
| NMTN-09917 | SAV1088 | | 1142169 | 1142831 | 1142405 | + | conserved hypothetical protein |
| NMTN-11316 | SAV1088 | | 1142169 | 1142831 | 1142689 | | |
| NMTN-01837 | SAV1089 | | 1143077 | 1144774 | 1143081 | − | conserved hypothetical protein |
| NMTN-01838 | SAV1089 | | 1143077 | 1144774 | 1143081 | | |
| NMTN-06252 | SAV1092 | | 1146390 | 1147016 | 1146420 | + | conserved hypothetical protein |
| NMTN-04761 | SAV1092 | | 1146390 | 1147016 | 1146826 | | |
| NMTN-08841c | SAV1092 | | 1146390 | 1147016 | 1146899 | | |
| NMTN-02453 | SAV1092 | | 1146390 | 1147016 | 1146952 | | |
| NMTN-10868 | SAV1094 | phdB | 1148303 | 1149280 | 1148326 | + | pyruvate dehydrogenase E1 component |
| | | | | | | | beta subunit |
| NMTN-14671 | SAV1096 | pdhD | 1150667 | 1152073 | 1151889 | + | dihydrolipoamide dehydrogenase |
| | | | | | | | component of pyruvate |
| | | | | | | | dehydrogenase E3 |
| NMTN-04312 | SAV1098 | | 1152660 | 1153199 | 1152673 | + | conserved hypothetical protein |
| NMTN-11380 | SAV1098 | | 1152660 | 1153199 | 1153045 | | |
| NMTN-08945 | SAV1098 | | 1152660 | 1153199 | 1153174 | | |
| NMTN-11872 | SAV1100 | potB | 1154299 | 1155096 | 1154309 | + | spermidine/putrescine ABC transporter |
| | | | | | | | homolog |
| NMTN-13250 | SAV1100 | potB | 1154299 | 1155096 | 1154351 | | |
| NMTN-11647d | SAV1100 | potB | 1154299 | 1155096 | 1154357 | | |
| NMTN-12516 | SAV1100 | potB | 1154299 | 1155096 | 1155032 | | |
| NMTN-12133 | SAV1101 | potC | 1155102 | 1155911 | 1155531 | + | spermidine/putrescine ABC transporter |
| | | | | | | | homolog |
| NMTN-13079 | SAV1102 | potD | 1155911 | 1156984 | 1155989 | + | spermidine/putrescine-binding protein |
| | | | | | | | precursor homolog |
| NMTN-02669 | SAV1102 | potD | 1155911 | 1156984 | 1156344 | | |
| NMTN-06164 | SAV1104 | | 1158360 | 1158779 | 1158409 | + | hypothetical protein |
| NMTN-06070 | SAV1105 | | 1158858 | 1160210 | 1159058 | − | hypothetical protein |
| NMTN-03662 | SAV1105 | | 1158858 | 1160210 | 1159882 | | |
| NMTN-08986 | SAV1108 | | 1162146 | 1162337 | 1162144 | − | hypothetical protein |
| NMTN-12645 | SAV1108 | | 1162146 | 1162337 | 1162219 | | |
| NMTN-02729 | SAV1109 | | 1162439 | 1164286 | 1162970 | + | GTP-binding elongation factor |
| | | | | | | | homologue |
| NMTN-04029 | SAV1109 | | 1162439 | 1164286 | 1163027 | | |
| NMTN-08961 | SAV1109 | | 1162439 | 1164286 | 1163361 | | |
| NMTN-12684 | SAV1109 | | 1162439 | 1164286 | 1163565 | | |
| NMTN-09092 | SAV1109 | | 1162439 | 1164286 | 1163754 | | |
| NMTN-03460 | SAV1109 | | 1162439 | 1164286 | 1163975 | | |
| NMTN-11571 | SAV1109 | | 1162439 | 1164286 | 1164012 | | |
| NMTN-06499 | SAV1110 | | 1164426 | 1164593 | 1164481 | − | conserved hypothetical protein |
| NMTN-04166 | SAV1111 | | 1164595 | 1165077 | 1164615 | − | conserved hypothetical protein |
| NMTN-06069 | SAV1111 | | 1164595 | 1165077 | 1164615 | | |
| NMTN-07725 | SAV1111 | | 1164595 | 1165077 | 1164720 | | |
| NMTN-01941 | SAV1111 | | 1164595 | 1165077 | 1164732 | | |
| NMTN-09768 | SAV1112 | | 1165218 | 1165493 | 1165303 | + | conserved hypothetical protein |
| NMTN-12949 | SAV1114 | pycA | 1167587 | 1171039 | 1167639 | + | pyruvate carboxylase |
| NMTN-03965 | SAV1114 | pycA | 1167587 | 1171039 | 1168983 | | |
| NMTN-04475 | SAV1114 | pycA | 1167587 | 1171039 | 1169007 | | |
| NMTN-02885 | SAV1114 | pycA | 1167587 | 1171039 | 1169344 | | |
| NMTN-07821 | SAV1114 | pycA | 1167587 | 1171039 | 1169483 | | |
| NMTN-03778 | SAV1114 | pycA | 1167587 | 1171039 | 1169690 | | |
| NMTN-06263 | SAV1114 | pycA | 1167587 | 1171039 | 1169840 | | |
| NMTN-06749 | SAV1114 | pycA | 1167587 | 1171039 | 1170523 | | |
| NMTN-14423 | SAV1115 | | 1171179 | 1172090 | 1171447 | − | hypothetical protein |
| NMTN-02477 | SAV1117 | | 1173466 | 1173927 | 1173740 | + | conserved hypothetical protein |
| NMTN-00441 | SAV1118 | | 1174254 | 1175291 | 1174272 | + | conserved hypothetical protein |
| NMTN-09283 | SAV1121 | | 1175802 | 1176728 | 1175912 | − | hypothetical protein |
| NMTN-01940 | SAV1121 | | 1175802 | 1176728 | 1176202 | | |
| NMTN-04298 | SAV1121 | | 1175802 | 1176728 | 1176307 | | |
| NMTN-03807 | SAV1121 | | 1175802 | 1176728 | 1176643 | | |
| NMTN-04820 | SAV1122 | | 1176967 | 1177221 | 1176992 | + | conserved hypothetical protein |
| NMTN-05249 | SAV1122 | | 1176967 | 1177221 | 1176994 | | |
| NMTN-07870 | SAV1122 | | 1176967 | 1177221 | 1177184 | | |
| NMTN-12232 | SAV1123 | | 1177224 | 1177613 | 1177529 | − | conserved hypothetical protein |
| NMTN-07969 | SAV1126 | | 1178771 | 1179910 | 1179699 | − | conserved hypothetical protein |
| NMTN-11795 | SAV1126 | | 1178771 | 1179910 | 1179764 | | |
| NMTN-06230 | SAV1127 | | 1180037 | 1180594 | 1180224 | + | conserved hypothetical protein |
| NMTN-11478 | SAV1127 | | 1180037 | 1180594 | 1180315 | | |
| NMTN-06622 | SAV1127 | | 1180037 | 1180594 | 1180356 | | |
| NMTN-10902 | SAV1127 | | 1180037 | 1180594 | 1180577 | | |
| NMTN-06127 | SAV1129 | | 1181001 | 1182938 | 1181169 | − | conserved hypothetical protein |
| NMTN-02671 | SAV1129 | | 1181001 | 1182938 | 1181660 | | |
| NMTN-06572 | SAV1129 | | 1181001 | 1182938 | 1181698 | | |
| NMTN-05059 | SAV1129 | | 1181001 | 1182938 | 1181880 | | |
| NMTN-04990 | SAV1129 | | 1181001 | 1182938 | 1181892 | | |
| NMTN-12075 | SAV1129 | | 1181001 | 1182938 | 1182881 | | |
| NMTN-12628 | SAV1130 | | 1183141 | 1184193 | 1183685 | − | cell surface protein |
| NMTN-01954 | SAV1130 | | 1183141 | 1184193 | 1184167 | | |
| NMTN-09284 | SAV1131 | | 1184402 | 1185085 | 1184758 | + | conserved hypothetical protein |
| NMTN-09597 | SAV1131 | | 1184402 | 1185085 | 1185000 | | |
| NMTN-12630 | SAV1131 | | 1184402 | 1185085 | 1185001 | | |
| NMTN-03054 | SAV1132 | | 1185085 | 1186161 | 1185121 | + | conserved hypothetical protein |
| NMTN-04382 | SAV1132 | | 1185085 | 1186161 | 1185241 | | |
| NMTN-03458 | SAV1133 | | 1186158 | 1187036 | 1186708 | + | hypothetical protein |
| NMTN-13865 | SAV1133 | | 1186158 | 1187036 | 1186776 | | |
| NMTN-12849 | SAV1134 | | 1187193 | 1188014 | 1187628 | + | hypothetical protein |
| NMTN-03811 | SAV1135 | | 1188076 | 1188810 | 1188101 | + | conserved hypothetical protein |
| NMTN-01454 | SAV1135 | | 1188076 | 1188810 | 1188401 | | |
| NMTN-11757 | SAV1135 | | 1188076 | 1188810 | 1188615 | | |
| NMTN-06585 | SAV1136 | | 1188838 | 1189152 | 1188874 | + | conserved hypothetical protein |
| NMTN-13688 | SAV1136 | | 1188838 | 1189152 | 1189129 | | |
| NMTN-05067 | SAV1136 | | 1188838 | 1189152 | 1189143 | | |
| NMTN-04124 | SAV1137 | | 1189536 | 1190276 | 1190143 | + | hypothetical protein |
| NMTN-06200 | SAV1142 | | 1196123 | 1196644 | 1196202 | + | conserved hypothetical protein |
| NMTN-11954 | SAV1142 | | 1196123 | 1196644 | 1196204 | | |
| NMTN-04007 | SAV1142 | | 1196123 | 1196644 | 1196408 | | |
| NMTN-11871 | SAV1143 | | 1196717 | 1198429 | 1196764 | + | DNA-dependent DNA polymerase beta |
| | | | | | | | chain |
| NMTN-11643 | SAV1143 | | 1196717 | 1198429 | 1197614 | | |
| NMTN-00668 | SAV1143 | | 1196717 | 1198429 | 1197690 | | |
| NMTN-06925 | SAV1143 | | 1196717 | 1198429 | 1197704 | | |
| NMTN-05571 | SAV1143 | | 1196717 | 1198429 | 1197903 | | |
| NMTN-05516 | SAV1143 | | 1196717 | 1198429 | 1197947 | | |
| NMTN-03259 | SAV1143 | | 1196717 | 1198429 | 1198100 | | |
| NMTN-02361 | SAV1143 | | 1196717 | 1198429 | 1198392 | | |
| NMTN-09072 | SAV1144 | mutS2 | 1198439 | 1200787 | 1198756 | + | MutS-like protein |
| NMTN-05614 | SAV1144 | mutS2 | 1198439 | 1200787 | 1199207 | | |
| NMTN-06846 | SAV1144 | mutS2 | 1198439 | 1200787 | 1199667 | | |
| NMTN-06694 | SAV1144 | mutS2 | 1198439 | 1200787 | 1199670 | | |
| NMTN-14509 | SAV1144 | mutS2 | 1198439 | 1200787 | 1200027 | | |
| NMTN-10908 | SAV1144 | mutS2 | 1198439 | 1200787 | 1200210 | | |
| NMTN-05356 | SAV1146 | uvrC | 1201598 | 1203379 | 1201647 | + | excinuclease ABC subunit C |
| NMTN-04650 | SAV1146 | uvrC | 1201598 | 1203379 | 1201693 | | |
| NMTN-01981 | SAV1146 | uvrC | 1201598 | 1203379 | 1201787 | | |
| NMTN-12597 | SAV1146 | uvrC | 1201598 | 1203379 | 1201873 | | |
| NMTN-05099 | SAV1146 | uvrC | 1201598 | 1203379 | 1202033 | | |
| NMTN-03025 | SAV1146 | uvrC | 1201598 | 1203379 | 1203080 | | |
| NMTN-06914 | SAV1147 | sdhC | 1203703 | 1204317 | 1203326 | + | succinate dehydrogenase cytochrome |
| | | | | | | | b-558 |
| NMTN-02502 | SAV1147 | sdhC | 1203703 | 1204317 | 1203845 | | |
| NMTN-10669 | SAV1147 | sdhC | 1203703 | 1204317 | 1203886 | | |
| NMTN-09169 | SAV1148 | sdhA | 1204369 | 1206135 | 1204726 | + | succinate dehydrogenase flavoprotein |
| | | | | | | | subunit |
| NMTN-12329 | SAV1148 | sdhA | 1204369 | 1206135 | 1204774 | | |
| NMTN-12873 | SAV1148 | sdhA | 1204369 | 1206135 | 1204777 | | |
| NMTN-02471 | SAV1148 | sdhA | 1204369 | 1206135 | 1205102 | | |
| NMTN-01442 | SAV1148 | sdhA | 1204369 | 1206135 | 1205296 | | |
| NMTN-07394 | SAV1150 | sdhB | 1206150 | 1206455 | 1206402 | + | iron-sulphur subunit of succinate |
| | | | | | | | dehydrogenase |
| NMTN-04085 | SAV1150 | sdhB | 1206359 | 1206949 | 1206782 | + | iron-sulphur subunit of succinate |
| | | | | | | | dehydrogenase |
| NMTN-02844 | SAV1152 | | 1207997 | 1208584 | 1208243 | + | conserved hypothetical protein |
| NMTN-11877 | SAV1153 | | 1208577 | 1209080 | 1208843 | + | conserved hypothetical protein |
| NMTN-05369 | SAV1153 | | 1208577 | 1209080 | 1208896 | | |
| NMTN-13803 | SAV1157 | | 1211338 | 1211844 | 1211576 | + | hypothetical protein |
| NMTN-03597 | SAV1158 | | 1212104 | 1212601 | 1212282 | + | hypothetical protein |
| NMTN-11961 | SAV1159 | | 1212751 | 1213101 | 1212934 | + | hypothetical protein |
| NMTN-11602 | SAV1163 | | 1216904 | 1217863 | 1217039 | − | alpha-hemolysin precursor |
| NMTN-02140 | SAV1163 | | 1216904 | 1217863 | 1217218 | | |
| NMTN-11832 | SAV1163 | | 1216904 | 1217863 | 1217451 | | |
| NMTN-11217 | SAV1163 | | 1216904 | 1217863 | 1217666 | | |
| NMTN-11568 | SAV1163 | | 1216904 | 1217863 | 1217751 | | |
| NMTN-01440 | SAV1166 | | 1219400 | 1220116 | 1219631 | − | hypothetical protein |
| NMTN-10229 | SAV1168 | | 1221044 | 1221769 | 1221223 | − | hypothetical protein |
| NMTN-11955 | SAV1168 | | 1221044 | 1221769 | 1221552 | | |
| NMTN-11698 | SAV1168 | | 1221044 | 1221769 | 1221633 | | |
| NMTN-05014 | SAV1169 | argF | 1222207 | 1223208 | 1222304 | + | ornithine carbamoyltransferase |
| NMTN-05018 | SAV1169 | argF | 1222207 | 1223208 | 1222304 | | |
| NMTN-05048 | SAV1169 | argF | 1222207 | 1223208 | 1222304 | | |
| NMTN-05050 | SAV1169 | argF | 1222207 | 1223208 | 1222304 | | |
| NMTN-04450 | SAV1169 | argF | 1222207 | 1223208 | 1222683 | | |
| NMTN-12097 | SAV1169 | argF | 1222207 | 1223208 | 1222967 | | |
| NMTN-12598 | SAV1170 | | 1223231 | 1224163 | 1223473 | + | hypothetical protein |
| NMTN-11736 | SAV1170 | | 1223231 | 1224163 | 1223799 | | |
| NMTN-11300 | SAV1170 | | 1223231 | 1224163 | 1223852 | | |
| NMTN-12554 | SAV1171 | | 1224335 | 1225891 | 1224728 | + | conserved hypothetical protein |
| NMTN-04829 | SAV1171 | | 1224335 | 1225891 | 1225356 | | |
| NMTN-04830 | SAV1171 | | 1224335 | 1225891 | 1225356 | | |
| NMTN-06689 | SAV1171 | | 1224335 | 1225891 | 1225875 | | |
| NMTN-04507 | SAV1172 | | 1226198 | 1226425 | 1226286 | + | hypothetical protein |
| NMTN-07029 | SAV1173 | | 1226731 | 1227678 | 1226742 | − | conserved hypothetical protein |
| NMTN-00372 | SAV1173 | | 1226731 | 1227678 | 1226763 | | |
| NMTN-06420 | SAV1173 | | 1226731 | 1227678 | 1227038 | | |
| NMTN-06204 | SAV1173 | | 1226731 | 1227678 | 1227064 | | |
| NMTN-12273 | SAV1173 | | 1226731 | 1227678 | 1227067 | | |
| NMTN-11641 | SAV1174 | | 1227927 | 1228115 | 1227948 | + | hypothetical protein |
| NMTN-07267 | SAV1174 | | 1227927 | 1228115 | 1227976 | | |
| NMTN-08529 | SAV1174 | | 1227927 | 1228115 | 1227976 | | |
| NMTN-08918 | SAV1175 | | 1229478 | 1230164 | 1229836 | + | conserved hypothetical protein |
| NMTN-08919 | SAV1175 | | 1229478 | 1230164 | 1229836 | | |
| NMTN-08920 | SAV1175 | | 1229478 | 1230164 | 1229836 | | |
| NMTN-08928 | SAV1175 | | 1229478 | 1230164 | 1229836 | | |
| NMTN-08929 | SAV1175 | | 1229478 | 1230164 | 1229836 | | |
| NMTN-08931 | SAV1175 | | 1229478 | 1230164 | 1229836 | | |
| NMTN-08932 | SAV1175 | | 1229478 | 1230164 | 1229836 | | |
| NMTN-08933 | SAV1175 | | 1229478 | 1230164 | 1229836 | | |
| NMTN-08934 | SAV1175 | | 1229478 | 1230164 | 1229836 | | |
| NMTN-08935 | SAV1175 | | 1229478 | 1230164 | 1229836 | | |
| NMTN-08936 | SAV1175 | | 1229478 | 1230164 | 1229836 | | |
| NMTN-08938 | SAV1175 | | 1229478 | 1230164 | 1229836 | | |
| NMTN-08939 | SAV1175 | | 1229478 | 1230164 | 1229836 | | |
| NMTN-08940 | SAV1175 | | 1229478 | 1230164 | 1229836 | | |
| NMTN-08941 | SAV1175 | | 1229478 | 1230164 | 1229836 | | |
| NMTN-08943 | SAV1175 | | 1229478 | 1230164 | 1229836 | | |
| NMTN-08944 | SAV1175 | | 1229478 | 1230164 | 1229836 | | |
| NMTN-08946 | SAV1175 | | 1229478 | 1230164 | 1229836 | | |
| NMTN-08947 | SAV1175 | | 1229478 | 1230164 | 1229836 | | |
| NMTN-08949 | SAV1175 | | 1229478 | 1230164 | 1229836 | | |
| NMTN-08950 | SAV1175 | | 1229478 | 1230164 | 1229836 | | |
| NMTN-08951 | SAV1175 | | 1229478 | 1230164 | 1229836 | | |
| NMTN-08952 | SAV1175 | | 1229478 | 1230164 | 1229836 | | |
| NMTN-08954 | SAV1175 | | 1229478 | 1230164 | 1229836 | | |
| NMTN-08955 | SAV1175 | | 1229478 | 1230164 | 1229836 | | |
| NMTN-08956 | SAV1175 | | 1229478 | 1230164 | 1229836 | | |
| NMTN-08957 | SAV1175 | | 1229478 | 1230164 | 1229836 | | |
| NMTN-08958 | SAV1175 | | 1229478 | 1230164 | 1229836 | | |
| NMTN-08959 | SAV1175 | | 1229478 | 1230164 | 1229836 | | |
| NMTN-09081 | SAV1175 | | 1229478 | 1230164 | 1229836 | | |
| NMTN-09082 | SAV1175 | | 1229478 | 1230164 | 1229836 | | |
| NMTN-09083 | SAV1175 | | 1229478 | 1230164 | 1229836 | | |
| NMTN-09091 | SAV1175 | | 1229478 | 1230164 | 1229836 | | |
| NMTN-09093 | SAV1175 | | 1229478 | 1230164 | 1229836 | | |
| NMTN-09095 | SAV1175 | | 1229478 | 1230164 | 1229836 | | |
| NMTN-09096 | SAV1175 | | 1229478 | 1230164 | 1229836 | | |
| NMTN-09097 | SAV1175 | | 1229478 | 1230164 | 1229836 | | |
| NMTN-09100 | SAV1175 | | 1229478 | 1230164 | 1229836 | | |
| NMTN-09101 | SAV1175 | | 1229478 | 1230164 | 1229836 | | |
| NMTN-09102 | SAV1175 | | 1229478 | 1230164 | 1229836 | | |
| NMTN-09103 | SAV1175 | | 1229478 | 1230164 | 1229836 | | |
| NMTN-09104 | SAV1175 | | 1229478 | 1230164 | 1229836 | | |
| NMTN-09105 | SAV1175 | | 1229478 | 1230164 | 1229836 | | |
| NMTN-09106 | SAV1175 | | 1229478 | 1230164 | 1229836 | | |
| NMTN-09109 | SAV1175 | | 1229478 | 1230164 | 1229836 | | |
| NMTN-06915 | SAV1175 | | 1229478 | 1230164 | 1230042 | | |
| NMTN-07410 | SAV1176 | | 1230268 | 1230714 | 1230612 | − | conserved hypothetical protein |
| NMTN-10530 | SAV1176 | | 1230268 | 1230714 | 1230726 | | |
| NMTN-12322 | SAV1177 | | 1230909 | 1232522 | 1231486 | + | conserved hypothetical protein |
| NMTN-07527 | SAV1177 | | 1230909 | 1232522 | 1231768 | | |
| NMTN-13027 | SAV1177 | | 1230909 | 1232522 | 1231838 | | |
| NMTN-04044 | SAV1177 | | 1230909 | 1232522 | 1231995 | | |
| NMTN-10949 | SAV1190 | | 1245659 | 1245949 | 1245660 | + | conserved hypothetical protein |
| NMTN-14291 | SAV1191 | | 1246200 | 1246838 | 1246431 | + | hypothetical protein |
| NMTN-12315 | SAV1191 | | 1246200 | 1246838 | 1246830 | | |
| NMTN-00715 | SAV1194 | | 1250744 | 1251541 | 1251295 | + | conserved hypothetical protein |
| NMTN-00857 | SAV1194 | | 1250744 | 1251541 | 1251433 | | |
| NMTN-02459 | SAV1196 | lsp | 1252365 | 1252856 | 1252794 | + | lipoprotein signal peptidase |
| NMTN-00656 | SAV1201 | pyrC | 1257152 | 1258426 | 1257410 | + | dihydroorotase |
| NMTN-02980 | SAV1201 | pyrC | 1257152 | 1258426 | 1258344 | | |
| NMTN-00609 | SAV1201 | pyrC | 1257152 | 1258426 | 1258423 | | |
| NMTN-11122 | SAV1202 | pyrAA | 1258428 | 1259528 | 1259074 | + | carbamoyl-phosphate synthase small |
| | | | | | | | chain |
| NMTN-00678 | SAV1202 | pyrAA | 1258428 | 1259528 | 1259466 | | |
| NMTN-02887 | SAV1203 | pyrAB | 1259521 | 1262694 | 1259715 | + | carbamoyl-phosphate synthase large |
| | | | | | | | chain |
| NMTN-04659 | SAV1203 | pyrAB | 1259521 | 1262694 | 1259715 | | |
| NMTN-05220 | SAV1203 | pyrAB | 1259521 | 1262694 | 1259715 | | |
| NMTN-03401 | SAV1203 | pyrAB | 1259521 | 1262694 | 1260071 | | |
| NMTN-04640 | SAV1203 | pyrAB | 1259521 | 1262694 | 1260211 | | |
| NMTN-02298 | SAV1203 | pyrAB | 1259521 | 1262694 | 1260215 | | |
| NMTN-13691 | SAV1203 | pyrAB | 1259521 | 1262694 | 1260327 | | |
| NMTN-10041 | SAV1203 | pyrAB | 1259521 | 1262694 | 1260470 | | |
| NMTN-05726 | SAV1203 | pyrAB | 1259521 | 1262694 | 1261633 | | |
| NMTN-05191 | SAV1203 | pyrAB | 1259521 | 1262694 | 1261835 | | |
| NMTN-08897 | SAV1203 | pyrAB | 1259521 | 1262694 | 1262037 | | |
| NMTN-12249 | SAV1204 | pyrF | 1262804 | 1263496 | 1262800 | + | orotidine-5-phosphate decarboxylase |
| NMTN-02904 | SAV1205 | pyrE | 1263496 | 1264107 | 1263564 | + | orotate phosphoribosyltransferase |
| NMTN-12091 | SAV1206 | | 1264137 | 1264349 | 1264167 | + | hypothetical protein |
| NMTN-03984 | SAV1206 | | 1264137 | 1264349 | 1264282 | | |
| NMTN-04888 | SAV1207 | | 1264786 | 1265187 | 1264966 | + | conserved hypothetical protein |
| NMTN-05132 | SAV1208 | | 1265450 | 1267147 | 1265761 | − | hypothetical protein |
| NMTN-00463 | SAV1208 | | 1265450 | 1267147 | 1265800 | | |
| NMTN-04917 | SAV1208 | | 1265450 | 1267147 | 1265955 | | |
| NMTN-13392 | SAV1208 | | 1265450 | 1267147 | 1266144 | | |
| NMTN-09798 | SAV1208 | | 1265450 | 1267147 | 1266753 | | |
| NMTN-09735 | SAV1213 | | 1272592 | 1273545 | 1273265 | + | hypothetical protein |
| NMTN-10522 | SAV1213 | | 1272592 | 1273545 | 1273363 | | |
| NMTN-00197 | SAV1217 | | 1275545 | 1276930 | 1275792 | + | hypothetical protein |
| NMTN-00392 | SAV1217 | | 1275545 | 1276930 | 1275792 | | |
| NMTN-10603 | SAV1217 | | 1275545 | 1276930 | 1276244 | | |
| NMTN-07201 | SAV1217 | | 1275545 | 1276930 | 1276466 | | |
| NMTN-00032 | SAV1218 | | 1276933 | 1278027 | 1276936 | + | conserved hypothetical protein |
| NMTN-14673d | SAV1219 | | 1278034 | 1278777 | 1278278 | + | conserved hypothetical protein |
| NMTN-10712 | SAV1220 | | 1278774 | 1280768 | 1280320 | + | protein kinase |
| NMTN-06272 | SAV1220 | | 1278774 | 1280768 | 1280509 | | |
| NMTN-03940 | SAV1220 | | 1278774 | 1280768 | 1280550 | | |
| NMTN-12600 | SAV1221 | | 1280996 | 1281871 | 1281812 | + | conserved hypothetical protein |
| NMTN-06693 | SAV1224 | rpmB | 1283545 | 1283733 | 1283538 | − | 50S ribosomal protein L28 |
| NMTN-12416 | SAV1224 | rpmB | 1283545 | 1283733 | 1283647 | | |
| NMTN-11428 | SAV1224 | rpmB | 1283545 | 1283733 | 1283651 | | |
| NMTN-00020 | SAV1226 | | 1284565 | 1286211 | 1284835 | + | conserved hypothetical protein |
| NMTN-11173 | SAV1227 | recG | 1286401 | 1288461 | 1287281 | + | ATP-dependent DNA helicase |
| NMTN-10001 | SAV1233 | rnc | 1292657 | 1293388 | 1293361 | + | RNase III |
| NMTN-10529 | SAV1234 | smc | 1293535 | 1297101 | 1297053 | + | chromosome segregation SMC protein |
| NMTN-11999 | SAV1240 | trmD | 1301464 | 1302201 | 1302184 | + | tRNA-mehtyltransferase |
| NMTN-09294 | SAV1241 | rplS | 1302305 | 1302655 | 1302627 | + | 50S ribosomal protein L19 |
| NMTN-13189 | SAV1242 | | 1302899 | 1305505 | 1302963 | − | conserved hypothetical protein |
| NMTN-00744 | SAV1242 | | 1302899 | 1305505 | 1303982 | | |
| NMTN-05179 | SAV1242 | | 1302899 | 1305505 | 1304880 | | |
| NMTN-06465 | SAV1242 | | 1302899 | 1305505 | 1305335 | | |
| NMTN-06435 | SAV1242 | | 1302899 | 1305505 | 1305460 | | |
| NMTN-01541 | SAV1244 | rnhB | 1306774 | 1307541 | 1306938 | + | RNase HII |
| NMTN-02806 | SAV1244 | rnhB | 1306774 | 1307541 | 1307114 | | |
| NMTN-02853 | SAV1244 | rnhB | 1306774 | 1307541 | 1307391 | | |
| NMTN-00594 | SAV1247 | lytN | 1309974 | 1311092 | 1310749 | + | LytN protein |
| NMTN-10497 | SAV1247 | lytN | 1309974 | 1311092 | 1310945 | | |
| NMTN-11340 | SAV1248 | fmhC | 1311120 | 1312364 | 1311487 | + | FmhC protein |
| | | (eprh) | | | | | |
| NMTN-12196 | SAV1248 | fmhC | 1311120 | 1312364 | 1311812 | | |
| | | (eprh) | | | | | |
| NMTN-11358 | SAV1248 | fmhC | 1311120 | 1312364 | 1311831 | | |
| | | (eprh) | | | | | |
| NMTN-12808 | SAV1248 | fmhC | 1311120 | 1312364 | 1311837 | | |
| | | (eprh) | | | | | |
| NMTN-02920 | SAV1248 | fmhC | 1311120 | 1312364 | 1311865 | | |
| | | (eprh) | | | | | |
| NMTN-12758 | SAV1248 | fmhC | 1311120 | 1312364 | 1311987 | | |
| | | (eprh) | | | | | |
| NMTN-10449 | SAV1248 | fmhC | 1311120 | 1312364 | 1312099 | | |
| | | (eprh) | | | | | |
| NMTN-06190 | SAV1249 | | 1312537 | 1313409 | 1312874 | + | hypothetical protein |
| NMTN-12222 | SAV1249 | | 1312537 | 1313409 | 1312917 | | |
| NMTN-09503 | SAV1249 | | 1312537 | 1313409 | 1313153 | | |
| NMTN-03537 | SAV1250 | | 1313589 | 1315658 | 1315620 | + | DNA topoisomerase I topA homologue |
| NMTN-03112 | SAV1250 | | 1313589 | 1315658 | 1315658 | | |
| NMTN-03407 | SAV1251 | gid | 1315814 | 1317121 | 1315967 | + | glucose-inhibited division protein gid |
| NMTN-01735 | SAV1251 | gid | 1315814 | 1317121 | 1316731 | | |
| NMTN-01736 | SAV1251 | gid | 1315814 | 1317121 | 1316731 | | |
| NMTN-09425 | SAV1270 | rbfA | 1339660 | 1340010 | 1339993 | + | ribosome-binding factor A |
| NMTN-02315 | SAV1271 | truB | 1340179 | 1341096 | 1340184 | + | tRNA pseudouridine 5S synthase |
| NMTN-13097 | SAV1271 | truB | 1340179 | 1341096 | 1340762 | | |
| NMTN-10021 | SAV1274 | pnpA | 1342835 | 1344931 | 1343059 | + | polyribonucleotide |
| | | | | | | | nucleotidyltransferase |
| NMTN-05451 | SAV1274 | pnpA | 1342835 | 1344931 | 1344453 | | |
| NMTN-12736 | SAV1274 | pnpA | 1342835 | 1344931 | 1344491 | | |
| NMTN-05375 | SAV1274 | pnpA | 1342835 | 1344931 | 1344627 | | |
| NMTN-04554 | SAV1274 | pnpA | 1342835 | 1344931 | 1344748 | | |
| NMTN-10926 | SAV1278 | | 1350212 | 1351477 | 1350412 | + | hypothetical protein |
| NMTN-09580 | SAV1278 | | 1350212 | 1351477 | 1350748 | | |
| NMTN-14030 | SAV1279 | | 1351477 | 1352763 | 1352044 | + | hypothetical protein |
| NMTN-04737 | SAV1280 | | 1352763 | 1353467 | 1352990 | + | hypothetical protein |
| NMTN-13632 | SAV1280 | | 1352763 | 1353467 | 1353336 | | |
| NMTN-14742 | SAV1280 | | 1352763 | 1353467 | 1353336 | | |
| NMTN-14747 | SAV1280 | | 1352763 | 1353467 | 1353336 | | |
| NMTN-08750 | SAV1281 | | 1353572 | 1354399 | 1353917 | + | conserved hypothetical protein |
| NMTN-08622 | SAV1281 | | 1353572 | 1354399 | 1354117 | | |
| NMTN-01812 | SAV1281 | | 1353572 | 1354399 | 1354355 | | |
| NMTN-10468 | SAV1284 | cinA | 1355646 | 1356797 | 1355941 | + | competence-damage inducible protein |
| NMTN-06397 | SAV1284 | cinA | 1355646 | 1356797 | 1356288 | | |
| NMTN-09773 | SAV1284 | cinA | 1355646 | 1356797 | 1356424 | | |
| NMTN-12157 | SAV1284 | cinA | 1355646 | 1356797 | 1356458 | | |
| NMTN-07371 | SAV1289 | | 1361541 | 1363301 | 1362828 | + | hypothetical protein |
| NMTN-07373 | SAV1289 | | 1361541 | 1363301 | 1362828 | | |
| NMTN-07375 | SAV1289 | | 1361541 | 1363301 | 1362828 | | |
| NMTN-07376 | SAV1289 | | 1361541 | 1363301 | 1362828 | | |
| NMTN-07379 | SAV1289 | | 1361541 | 1363301 | 1362828 | | |
| NMTN-00206 | SAV1292 | | 1364689 | 1366233 | 1365344 | + | conserved hypothetical protein |
| NMTN-09545 | SAV1294 | | 1366626 | 1367117 | 1366879 | + | conserved hypothetical protein |
| NMTN-09387 | SAV1294 | | 1366626 | 1367117 | 1366891 | | |
| NMTN-04775 | SAV1296 | mutS | 1367420 | 1370039 | 1367639 | + | DNA mismatch repair protein |
| NMTN-00866 | SAV1296 | mutS | 1367420 | 1370039 | 1367872 | | |
| NMTN-08510 | SAV1296 | mutS | 1367420 | 1370039 | 1367953 | | |
| NMTN-09099 | SAV1296 | mutS | 1367420 | 1370039 | 1368125 | | |
| NMTN-12900 | SAV1296 | mutS | 1367420 | 1370039 | 1368125 | | |
| NMTN-06111 | SAV1296 | mutS | 1367420 | 1370039 | 1368753 | | |
| NMTN-02925 | SAV1296 | mutS | 1367420 | 1370039 | 1368850 | | |
| NMTN-06063 | SAV1296 | mutS | 1367420 | 1370039 | 1369035 | | |
| NMTN-02609 | SAV1297 | mutL | 1370052 | 1372061 | 1370809 | + | DNA mismatch repair protein |
| NMTN-01512 | SAV1297 | mutL | 1370052 | 1372061 | 1370908 | | |
| NMTN-04409 | SAV1297 | mutL | 1370052 | 1372061 | 1370928 | | |
| NMTN-13276 | SAV1297 | mutL | 1370052 | 1372061 | 1371239 | | |
| NMTN-09150 | SAV1297 | mutL | 1370052 | 1372061 | 1371642 | | |
| NMTN-11299 | SAV1297 | mutL | 1370052 | 1372061 | 1371814 | | |
| NMTN-00722 | SAV1298 | glpP | 1372076 | 1372609 | 1372217 | + | glycerol uptake operon antiterminator |
| | | | | | | | regulatory protein |
| NMTN-06061 | SAV1298 | glpP | 1372076 | 1372609 | 1372311 | | |
| NMTN-03936 | SAV1298 | glpP | 1372076 | 1372609 | 1372454 | | |
| NMTN-11481 | SAV1300 | glpF | 1373084 | 1373902 | 1373099 | + | glycerol uptake facilitator |
| NMTN-11671 | SAV1300 | glpF | 1373084 | 1373902 | 1373099 | | |
| NMTN-10519 | SAV1301 | glpK | 1374031 | 1375527 | 1374048 | + | glycerol kinase |
| NMTN-08840 | SAV1301 | glpK | 1374031 | 1375527 | 1374822 | | |
| NMTN-05496 | SAV1301 | glpK | 1374031 | 1375527 | 1375475 | | |
| NMTN-02025 | SAV1302 | glpD | 1375637 | 1377358 | 1375894 | + | aerobic glycerol-3-phosphate |
| | | | | | | | dehydrogenase |
| NMTN-09995 | SAV1302 | glpD | 1375637 | 1377358 | 1376256 | | |
| NMTN-13128d | SAV1302 | glpD | 1375637 | 1377358 | 1376580 | | |
| NMTN-13156d | SAV1302 | glpD | 1375637 | 1377358 | 1376580 | | |
| NMTN-03791 | SAV1302 | glpD | 1375637 | 1377358 | 1376626 | | |
| NMTN-06177 | SAV1302 | glpD | 1375637 | 1377358 | 1376647 | | |
| NMTN-06192 | SAV1302 | glpD | 1375637 | 1377358 | 1376647 | | |
| NMTN-06197 | SAV1302 | glpD | 1375637 | 1377358 | 1376647 | | |
| NMTN-06199 | SAV1302 | glpD | 1375637 | 1377358 | 1376647 | | |
| NMTN-14027 | SAV1302 | glpD | 1375637 | 1377358 | 1376778 | | |
| NMTN-06537 | SAV1302 | glpD | 1375637 | 1377358 | 1377026 | | |
| NMTN-03553 | SAV1302 | glpD | 1375637 | 1377358 | 1377286 | | |
| NMTN-09695 | SAV1303 | | 1377508 | 1378422 | 1378047 | + | hypothetical protein |
| NMTN-09861 | SAV1303 | | 1377508 | 1378422 | 1378047 | | |
| NMTN-09845 | SAV1303 | | 1377508 | 1378422 | 1378102 | | |
| NMTN-06616 | SAV1305 | | 1379390 | 1379623 | 1379515 | + | hypothetical protein |
| NMTN-09936 | SAV1306 | bsaA | 1379844 | 1380320 | 1379863 | − | glutathione peroxidase |
| NMTN-09947 | SAV1306 | bsaA | 1379844 | 1380320 | 1379863 | | |
| NMTN-10045 | SAV1306 | bsaA | 1379844 | 1380320 | 1379863 | | |
| NMTN-12421 | SAV1306 | bsaA | 1379844 | 1380320 | 1380158 | | |
| NMTN-01741 | SAV1306 | bsaA | 1379844 | 1380320 | 1380256 | | |
| NMTN-04164 | SAV1306 | bsaA | 1379844 | 1380320 | 1380263 | | |
| NMTN-11511 | SAV1307 | | 1380432 | 1381670 | 1380428 | + | hypothetical protein |
| NMTN-00270 | SAV1307 | | 1380432 | 1381670 | 1380589 | | |
| NMTN-07320 | SAV1307 | | 1380432 | 1381670 | 1381621 | | |
| NMTN-07044 | SAV1308 | | 1381689 | 1382927 | 1381830 | + | hypothetical protein |
| NMTN-02357 | SAV1308 | | 1381689 | 1382927 | 1382922 | | |
| NMTN-01862 | SAV1314 | | 1390138 | 1390332 | 1390155 | + | hypothetical protein |
| NMTN-00442 | SAV1314 | | 1390138 | 1390332 | 1390287 | | |
| NMTN-13465 | SAV1315 | | 1390876 | 1391901 | 1390898 | − | conserved hypothetical protein |
| NMTN-03189 | SAV1315 | | 1390876 | 1391901 | 1391019 | | |
| NMTN-03808 | SAV1315 | | 1390876 | 1391901 | 1391298 | | |
| NMTN-12629 | SAV1315 | | 1390876 | 1391901 | 1391394 | | |
| NMTN-11842 | SAV1315 | | 1390876 | 1391901 | 1391480 | | |
| NMTN-07019 | SAV1317 | | 1392389 | 1393906 | 1392412 | | |
| NMTN-13732 | SAV1317 | | 1392389 | 1393906 | 1392510 | + | cardiolipin synthetase homolog |
| NMTN-00201 | SAV1317 | | 1392389 | 1393906 | 1392904 | | |
| NMTN-04854 | SAV1318 | | 1394090 | 1394587 | 1394303 | + | ABC transporter homolog |
| NMTN-12468 | SAV1318 | | 1394090 | 1394587 | 1394569 | | |
| NMTN-09053 | SAV1319 | | 1394612 | 1394989 | 1394776 | + | ABC transporter homolog |
| NMTN-12683 | SAV1319 | | 1394612 | 1394989 | 1394961 | | |
| NMTN-12683 | SAV1320 | | 1394958 | 1395689 | 1394961 | + | hypothetical protein |
| NMTN-06285 | SAV1320 | | 1394958 | 1395689 | 1395112 | | |
| NMTN-09497 | SAV1320 | | 1394958 | 1395689 | 1395570 | | |
| NMTN-06746 | SAV1321 | | 1395693 | 1396784 | 1395800 | + | hypothetical protein |
| NMTN-11164 | SAV1321 | | 1395693 | 1396784 | 1395865 | | |
| NMTN-08492 | SAV1321 | | 1395693 | 1396784 | 1396199 | | |
| NMTN-01824 | SAV1321 | | 1395693 | 1396784 | 1396558 | | |
| NMTN-11466 | SAV1322 | | 1396781 | 1397383 | 1396849 | + | hypothetical protein |
| NMTN-00021 | SAV1323 | | 1397496 | 1397684 | 1397665 | − | hypothetical protein |
| NMTN-03258 | SAV1324 | nuc | 1397823 | 1398356 | 1397880 | + | thermonuclease |
| NMTN-06615 | SAV1324 | nuc | 1397823 | 1398356 | 1397880 | | |
| NMTN-12295 | SAV1324 | nuc | 1397823 | 1398356 | 1398202 | | |
| NMTN-14502 | SAV1326 | | 1399671 | 1400261 | 1399714 | + | hypothetical protein |
| NMTN-12506 | SAV1326 | | 1399671 | 1400261 | 1399885 | | |
| NMTN-09502 | SAV1326 | | 1399671 | 1400261 | 1399955 | | |
| NMTN-08589 | SAV1326 | | 1399671 | 1400261 | 1400029 | | |
| NMTN-11769 | SAV1326 | | 1399671 | 1400261 | 1400035 | | |
| NMTN-04891 | SAV1326 | | 1399671 | 1400261 | 1400121 | | |
| NMTN-07790 | SAV1327 | | 1400315 | 1401697 | 1400366 | − | aspartate kinase homolog |
| NMTN-13143 | SAV1327 | | 1400315 | 1401697 | 1401124 | | |
| NMTN-03196 | SAV1327 | | 1400315 | 1401697 | 1401127 | | |
| NMTN-05492 | SAV1327 | | 1400315 | 1401697 | 1401128 | | |
| NMTN-11351 | SAV1327 | | 1400315 | 1401697 | 1401223 | | |
| NMTN-09599 | SAV1327 | | 1400315 | 1401697 | 1401352 | | |
| NMTN-11942 | SAV1328 | dhoM | 1401888 | 1403168 | 1401982 | + | homoserine dehydrogenase |
| NMTN-12625 | SAV1329 | thrC | 1403174 | 1404235 | 1403597 | + | threonine synthase |
| NMTN-09336 | SAV1330 | thrB | 1404237 | 1405151 | 1404938 | + | homoserine kinase homolog |
| NMTN-11455 | SAV1331 | | 1405209 | 1406012 | 1405348 | + | conserved hypothetical protein |
| NMTN-10079 | SAV1331 | | 1405209 | 1406012 | 1405960 | | |
| NMTN-00210 | SAV1332 | | 1406305 | 1406619 | 1406361 | − | hypothetical protein |
| NMTN-03920 | SAV1333 | | 1406837 | 1408291 | 1407265 | − | gamma-aminobutyrate permease |
| NMTN-08285 | SAV1333 | | 1406837 | 1408291 | 1407275 | | |
| NMTN-12837 | SAV1333 | | 1406837 | 1408291 | 1407499 | | |
| NMTN-07353 | SAV1334 | katA | 1408489 | 1410012 | 1409716 | + | catalase |
| NMTN-12739 | SAV1335 | rpmG | 1410103 | 1410252 | 1410230 | + | 50S ribosomal protein L33 |
| NMTN-06121 | SAV1336 | rpsN | 1410706 | 1410975 | 1410784 | + | 30S ribosomal protein S14 homolog |
| NMTN-10812 | SAV1336 | rpsN | 1410706 | 1410975 | 1410914 | | |
| NMTN-10673 | SAV1337 | | 1411132 | 1412109 | 1411162 | + | hypothetical protein |
| NMTN-03569 | SAV1337 | | 1411132 | 1412109 | 1411547 | | |
| NMTN-05206 | SAV1337 | | 1411132 | 1412109 | 1411713 | | |
| NMTN-13756 | SAV1337 | | 1411132 | 1412109 | 1411822 | | |
| NMTN-08926 | SAV1337 | | 1411132 | 1412109 | 1411890 | | |
| NMTN-12890 | SAV1337 | | 1411132 | 1412109 | 1411964 | | |
| NMTN-05032 | SAV1338 | | 1412131 | 1413147 | 1412387 | + | conserved hypothetical protein |
| NMTN-05025 | SAV1338 | | 1412131 | 1413147 | 1412390 | | |
| NMTN-12269 | SAV1338 | | 1412131 | 1413147 | 1412484 | | |
| NMTN-06936 | SAV1338 | | 1412131 | 1413147 | 1412612 | | |
| NMTN-08135 | SAV1338 | | 1412131 | 1413147 | 1412854 | | |
| NMTN-06117 | SAV1338 | | 1412131 | 1413147 | 1412867 | | |
| NMTN-04545 | SAV1340 | | 1414293 | 1414526 | 1414328 | + | hypothetical protein |
| NMTN-10517 | SAV1341 | | 1414663 | 1414902 | 1414726 | + | conserved hypothetical protein |
| NMTN-09392 | SAV1344 | | 1417715 | 1418182 | 1418148 | + | conserved hypothetical protein |
| NMTN-05227 | SAV1345 | | 1418305 | 1419426 | 1418338 | + | hypothetical protein |
| NMTN-03935 | SAV1345 | | 1418305 | 1419426 | 1418474 | | |
| NMTN-11156 | SAV1345 | | 1418305 | 1419426 | 1419070 | | |
| NMTN-13069 | SAV1346 | | 1419430 | 1422459 | 1419732 | + | hypothetical protein |
| NMTN-11888 | SAV1346 | | 1419430 | 1422459 | 1419734 | | |
| NMTN-00220 | SAV1346 | | 1419430 | 1422459 | 1421693 | | |
| NMTN-00491 | SAV1346 | | 1419430 | 1422459 | 1421693 | | |
| NMTN-08249 | SAV1346 | | 1419430 | 1422459 | 1421747 | | |
| NMTN-03758 | SAV1346 | | 1419430 | 1422459 | 1421901 | | |
| NMTN-07844 | SAV1346 | | 1419430 | 1422459 | 1422158 | | |
| NMTN-06464 | SAV1349 | opuD | 1423662 | 1424741 | 1423937 | + | glycine betaine transporter |
| NMTN-06752 | SAV1349 | opuD | 1423662 | 1424741 | 1424211 | | |
| NMTN-12482 | SAV1349 | opuD | 1423662 | 1424741 | 1424713 | | |
| NMTN-14562 | SAV1350 | citB | 1425688 | 1428393 | 1425998 | + | aconitate hydratase |
| NMTN-08221 | SAV1350 | citB | 1425688 | 1428393 | 1426027 | | |
| NMTN-07535 | SAV1350 | citB | 1425688 | 1428393 | 1426307 | | |
| NMTN-13526 | SAV1350 | citB | 1425688 | 1428393 | 1426658 | | |
| NMTN-00263 | SAV1350 | citB | 1425688 | 1428393 | 1427455 | | |
| NMTN-12465 | SAV1350 | citB | 1425688 | 1428393 | 1427527 | | |
| NMTN-02780 | SAV1350 | citB | 1425688 | 1428393 | 1427631 | | |
| NMTN-06317 | SAV1351 | | 1428573 | 1429040 | 1429017 | + | conserved hypothetical protein |
| NMTN-11868 | SAV1355 | parC | 1432724 | 1435126 | 1434834 | + | topoisomerase IV subunit A |
| NMTN-09342 | SAV1356 | alsT | 1435376 | 1436836 | 1435772 | + | amino acid carrier protein |
| NMTN-09110 | SAV1356 | alsT | 1435376 | 1436836 | 1436084 | | |
| NMTN-11968 | SAV1357 | glcT | 1437336 | 1438187 | 1437366 | + | transcription antiterminator |
| NMTN-11405 | SAV1357 | glcT | 1437336 | 1438187 | 1437394 | | |
| NMTN-14381 | SAV1357 | glcT | 1437336 | 1438187 | 1437691 | | |
| NMTN-11953 | SAV1361 | msrA | 1442736 | 1443245 | 1443044 | − | peptide methionine sulfoxide reductase |
| | | | | | | | homologue |
| NMTN-08767 | SAV1361 | msrA | 1442736 | 1443245 | 1443120 | | |
| NMTN-11797 | SAV1362 | msrR | 1443383 | 1444366 | 1443371 | + | peptide methionine sulfoxide reductase |
| | | | | | | | regulator |
| NMTN-09419 | SAV1362 | msrR | 1443383 | 1444366 | 1443936 | | |
| NMTN-04469 | SAV1364 | | 1444787 | 1446049 | 1444791 | + | hypothetical protein |
| NMTN-10415 | SAV1364 | | 1444787 | 1446049 | 1445427 | | |
| NMTN-10263 | SAV1364 | | 1444787 | 1446049 | 1445436 | | |
| NMTN-05518 | SAV1364 | | 1444787 | 1446049 | 1445739 | | |
| NMTN-03640 | SAV1364 | | 1444787 | 1446049 | 1445778 | | |
| NMTN-09431 | SAV1365 | tyrA | 1446186 | 1447277 | 1446275 | − | prephenate dehydrogenase |
| NMTN-10030 | SAV1365 | tyrA | 1446186 | 1447277 | 1446685 | | |
| NMTN-08757 | SAV1365 | tyrA | 1446186 | 1447277 | 1447043 | | |
| NMTN-09750 | SAV1365 | tyrA | 1446186 | 1447277 | 1447086 | | |
| NMTN-08643 | SAV1365 | tyrA | 1446186 | 1447277 | 1447138 | | |
| NMTN-10614 | SAV1366 | | 1447442 | 1448473 | 1447616 | + | hypothetical protein |
| NMTN-03998 | SAV1366 | | 1447442 | 1448473 | 1447944 | | |
| NMTN-05435 | SAV1366 | | 1447442 | 1448473 | 1448277 | | |
| NMTN-07306 | SAV1366 | | 1447442 | 1448473 | 1448300 | | |
| NMTN-00553 | SAV1367 | | 1448965 | 1450371 | 1449180 | + | hypothetical protein |
| NMTN-11322 | SAV1367 | | 1448965 | 1450371 | 1449347 | | |
| NMTN-11402 | SAV1367 | | 1448965 | 1450371 | 1449406 | | |
| NMTN-10864 | SAV1367 | | 1448965 | 1450371 | 1449474 | | |
| NMTN-00219 | SAV1367 | | 1448965 | 1450371 | 1449764 | | |
| NMTN-08852 | SAV1367 | | 1448965 | 1450371 | 1449767 | | |
| NMTN-09377 | SAV1367 | | 1448965 | 1450371 | 1450050 | | |
| NMTN-09383 | SAV1367 | | 1448965 | 1450371 | 1450050 | | |
| NMTN-02467 | SAV1367 | | 1448965 | 1450371 | 1450204 | | |
| NMTN-04639 | SAV1367 | | 1448965 | 1450371 | 1450266 | | |
| NMTN-09333 | SAV1368 | trpG | 1450368 | 1450934 | 1450403 | + | anthranilate synthase component II |
| NMTN-12242 | SAV1368 | trpG | 1450368 | 1450934 | 1450406 | | |
| NMTN-06482 | SAV1368 | trpG | 1450368 | 1450934 | 1450848 | | |
| NMTN-03894 | SAV1369 | trpD | 1450940 | 1451938 | 1451013 | + | anthranilate phosphoribosyltransferase |
| NMTN-01859 | SAV1369 | trpD | 1450940 | 1451938 | 1451028 | | |
| NMTN-11749 | SAV1369 | trpD | 1450940 | 1451938 | 1451105 | | |
| NMTN-02572 | SAV1369 | trpD | 1450940 | 1451938 | 1451259 | | |
| NMTN-09521 | SAV1369 | trpD | 1450940 | 1451938 | 1451321 | | |
| NMTN-02144 | SAV1369 | trpD | 1450940 | 1451938 | 1451883 | | |
| NMTN-10240 | SAV1370 | trpC | 1451940 | 1452722 | 1451994 | + | indole-3-glycerol phosphate synthase |
| NMTN-13128 | SAV1370 | trpC | 1451940 | 1452722 | 1452088 | | |
| NMTN-12365 | SAV1370 | trpC | 1451940 | 1452722 | 1452504 | | |
| NMTN-01453 | SAV1371 | trpF | 1452722 | 1453354 | 1452887 | + | phosphoriborylanthranilate isomerase |
| NMTN-10638 | SAV1371 | trpF | 1452722 | 1453354 | 1453088 | | |
| NMTN-10583 | SAV1371 | trpF | 1452722 | 1453354 | 1453131 | | |
| NMTN-15232 | SAV1371 | trpF | 1452722 | 1453354 | 1453274 | | |
| NMTN-13136 | SAV1371 | trpF | 1452722 | 1453354 | 1453338 | | |
| NMTN-11658 | SAV1372 | trpB | 1453347 | 1454561 | 1453397 | + | tryptophan synthase beta chain |
| NMTN-11372 | SAV1372 | trpB | 1453347 | 1454561 | 1453908 | | |
| NMTN-12217 | SAV1372 | trpB | 1453347 | 1454561 | 1454401 | | |
| NMTN-07688 | SAV1373 | trpA | 1454554 | 1455282 | 1454781 | + | tryptophan synthase alpha chain |
| NMTN-03755 | SAV1373 | trpA | 1454554 | 1455282 | 1454783 | | |
| NMTN-00727 | SAV1373 | trpA | 1454554 | 1455282 | 1454787 | | |
| NMTN-11591 | SAV1373 | trpA | 1454554 | 1455282 | 1454813 | | |
| NMTN-08044 | SAV1377 | | 1459409 | 1460176 | 1460055 | + | conserved hypothetical protein |
| NMTN-09532 | SAV1377 | | 1459409 | 1460176 | 1460145 | | |
| NMTN-09617 | SAV1378 | | 1460224 | 1460697 | 1460700 | − | hypothetical protein |
| NMTN-14539 | SAV1379 | | 1460821 | 1461522 | 1460852 | − | oligopeptide transporter putative |
| | | | | | | | ATPase domain |
| NMTN-00134 | SAV1379 | | 1460821 | 1461522 | 1460988 | | |
| NMTN-06686 | SAV1379 | | 1460821 | 1461522 | 1461163 | | |
| NMTN-04936 | SAV1379 | | 1460821 | 1461522 | 1461224 | | |
| NMTN-10028 | SAV1379 | | 1460821 | 1461522 | 1461303 | | |
| NMTN-08400 | SAV1379 | | 1460821 | 1461522 | 1461437 | | |
| NMTN-12518 | SAV1380 | | 1461515 | 1462288 | 1461524 | − | oligopeptide transport ATPase |
| NMTN-12101 | SAV1381 | | 1462275 | 1463105 | 1462572 | − | oligopeptide transporter membrane |
| | | | | | | | permease domain |
| NMTN-00217 | SAV1381 | | 1462275 | 1463105 | 1462639 | | |
| NMTN-05051 | SAV1381 | | 1462275 | 1463105 | 1462639 | | |
| NMTN-09250 | SAV1381 | | 1462275 | 1463105 | 1462879 | | |
| NMTN-05892 | SAV1382 | | 1463098 | 1464084 | 1463352 | − | oligopeptide transporter membrane |
| | | | | | | | permease domain |
| NMTN-11061 | SAV1382 | | 1463098 | 1464084 | 1463431 | | |
| NMTN-03860 | SAV1382 | | 1463098 | 1464084 | 1463586 | | |
| NMTN-04334 | SAV1382 | | 1463098 | 1464084 | 1463586 | | |
| NMTN-12330 | SAV1382 | | 1463098 | 1464084 | 1463586 | | |
| NMTN-04189 | SAV1382 | | 1463098 | 1464084 | 1464011 | | |
| NMTN-02100 | SAV1383 | | 1464388 | 1464732 | 1464513 | − | hypothetical protein |
| NMTN-00562 | SAV1383 | | 1464388 | 1464732 | 1464549 | | |
| NMTN-03893 | SAV1384 | | 1464937 | 1466751 | 1465073 | + | hypothetical protein |
| NMTN-04696 | SAV1384 | | 1464937 | 1466751 | 1465235 | | |
| NMTN-05049 | SAV1384 | | 1464937 | 1466751 | 1465238 | | |
| NMTN-11139 | SAV1384 | | 1464937 | 1466751 | 1465344 | | |
| NMTN-04664 | SAV1384 | | 1464937 | 1466751 | 1465924 | | |
| NMTN-09853 | SAV1384 | (?) | 1464937 | 1466751 | 1466405 | | |
| NMTN-00092 | SAV1385 | | 1466891 | 1467532 | 1466982 | − | hypothetical protein |
| NMTN-05416 | SAV1385 | | 1466891 | 1467532 | 1466983 | | |
| NMTN-00589 | SAV1385 | | 1466891 | 1467532 | 1467029 | | |
| NMTN-05195 | SAV1386 | pstB | 1467539 | 1468390 | 1467855 | − | phosphate ABC transporter |
| NMTN-09921 | SAV1386 | pstB | 1467539 | 1468390 | 1468057 | | |
| NMTN-14434 | SAV1386 | pstB | 1467539 | 1468390 | 1468189 | | |
| NMTN-05321 | SAV1386 | pstB | 1467539 | 1468390 | 1468230 | | |
| NMTN-15249 | SAV1386 | pstB | 1467539 | 1468390 | 1468296 | | |
| NMTN-10977 | SAV1387 | | 1468437 | 1469354 | 1469084 | − | hypothetical protein |
| NMTN-05589 | SAV1387 | | 1468437 | 1469354 | 1469218 | | |
| NMTN-12326 | SAV1387 | | 1468437 | 1469354 | 1469242 | | |
| NMTN-13757d | SAV1388 | | 1469356 | 1470282 | 1469676 | − | hypothetical protein |
| NMTN-06387 | SAV1388 | | 1469356 | 1470282 | 1470153 | | |
| NMTN-01659 | SAV1389 | | 1470473 | 1471456 | 1470660 | − | thioredoxine reductase |
| NMTN-01660 | SAV1389 | | 1470473 | 1471456 | 1470660 | | |
| NMTN-01661 | SAV1389 | | 1470473 | 1471456 | 1470660 | | |
| NMTN-01663 | SAV1389 | | 1470473 | 1471456 | 1470660 | | |
| NMTN-01665 | SAV1389 | | 1470473 | 1471456 | 1470660 | | |
| NMTN-01667 | SAV1389 | | 1470473 | 1471456 | 1470660 | | |
| NMTN-01668 | SAV1389 | | 1470473 | 1471456 | 1470660 | | |
| NMTN-03202 | SAV1389 | | 1470473 | 1471456 | 1471043 | | |
| NMTN-02833 | SAV1389 | | 1470473 | 1471456 | 1471072 | | |
| NMTN-04987 | SAV1391 | | 1472431 | 1473333 | 1472514 | − | conserved hypothetical protein |
| NMTN-06216 | SAV1391 | | 1472431 | 1473333 | 1472929 | | |
| NMTN-12430 | SAV1392 | | 1473480 | 1475081 | 1473754 | + | ABC transporter homolog |
| NMTN-04444 | SAV1392 | | 1473480 | 1475081 | 1474134 | | |
| NMTN-10598 | SAV1392 | | 1473480 | 1475081 | 1474930 | | |
| NMTN-07886 | SAV1393 | lysC | 1476143 | 1477348 | 1476809 | + | aspartokinase II |
| NMTN-09077 | SAV1393 | lysC | 1476143 | 1477348 | 1476878 | | |
| NMTN-11396 | SAV1393 | lysC | 1476143 | 1477348 | 1476977 | | |
| NMTN-12395 | SAV1394 | asd | 1477412 | 1478401 | 1477448 | + | aspartate semialdehyde dehydrogenase |
| NMTN-04943 | SAV1394 | asd | 1477412 | 1478401 | 1477616 | | |
| NMTN-03495 | SAV1394 | asd | 1477412 | 1478401 | 1477688 | | |
| NMTN-13095 | SAV1394 | asd | 1477412 | 1478401 | 1477767 | | |
| NMTN-10556 | SAV1395 | dapA | 1478403 | 1479290 | 1478460 | + | dihydrodipicolinate synthase |
| NMTN-09171 | SAV1395 | dapA | 1478403 | 1479290 | 1478610 | | |
| NMTN-13002 | SAV1395 | dapA | 1478403 | 1479290 | 1478611 | | |
| NMTN-02057 | SAV1395 | dapA | 1478403 | 1479290 | 1478686 | | |
| NMTN-12262 | SAV1395 | dapA | 1478403 | 1479290 | 1478880 | | |
| NMTN-09152 | SAV1395 | dapA | 1478403 | 1479290 | 1479088 | | |
| NMTN-06502 | SAV1395 | dapA | 1478403 | 1479290 | 1479254 | | |
| NMTN-00844 | SAV1396 | dapB | 1479287 | 1480009 | 1479993 | + | dihydrodipicolinate reductase |
| NMTN-12571 | SAV1397 | dapD | 1480036 | 1480755 | 1480075 | + | tetrahydrodipicolinate acetyltransferase |
| NMTN-13105 | SAV1397 | dapD | 1480036 | 1480755 | 1480474 | | |
| NMTN-07808 | SAV1398 | | 1480898 | 1482049 | 1481071 | + | hippurate hydrolase |
| NMTN-07856 | SAV1398 | | 1480898 | 1482049 | 1481749 | | |
| NMTN-11121 | SAV1399 | | 1482054 | 1483139 | 1482996 | + | hypothetical protein |
| NMTN-11130 | SAV1399 | | 1482054 | 1483139 | 1482996 | | |
| NMTN-06259 | SAV1400 | lysA | 1483129 | 1484394 | 1483631 | + | diaminopimelate decarboxylase |
| NMTN-06489 | SAV1400 | lysA | 1483129 | 1484394 | 1483713 | | |
| NMTN-10233 | SAV1400 | lysA | 1483129 | 1484394 | 1483890 | | |
| NMTN-10470 | SAV1400 | lysA | 1483129 | 1484394 | 1483935 | | |
| NMTN-13776 | SAV1400 | lysA | 1483129 | 1484394 | 1483958 | | |
| NMTN-13782 | SAV1400 | lysA | 1483129 | 1484394 | 1483958 | | |
| NMTN-13783 | SAV1400 | lysA | 1483129 | 1484394 | 1483958 | | |
| NMTN-13789 | SAV1400 | lysA | 1483129 | 1484394 | 1483958 | | |
| NMTN-13790 | SAV1400 | lysA | 1483129 | 1484394 | 1483958 | | |
| NMTN-13791 | SAV1400 | lysA | 1483129 | 1484394 | 1483958 | | |
| NMTN-13792 | SAV1400 | lysA | 1483129 | 1484394 | 1483958 | | |
| NMTN-13794 | SAV1400 | lysA | 1483129 | 1484394 | 1483958 | | |
| NMTN-13801 | SAV1400 | lysA | 1483129 | 1484394 | 1483958 | | |
| NMTN-13804 | SAV1400 | lysA | 1483129 | 1484394 | 1483958 | | |
| NMTN-13811 | SAV1400 | lysA | 1483129 | 1484394 | 1483958 | | |
| NMTN-13920 | SAV1400 | lysA | 1483129 | 1484394 | 1483958 | | |
| NMTN-08157 | SAV1400 | lysA | 1483129 | 1484394 | 1484002 | | |
| NMTN-10386 | SAV1400 | lysA | 1483129 | 1484394 | 1484192 | | |
| NMTN-11703 | SAV1401 | | 1484634 | 1485035 | 1484700 | − | hypothetical protein |
| NMTN-05537 | SAV1401 | | 1484634 | 1485035 | 1484787 | | |
| NMTN-12337 | SAV1401 | | 1484634 | 1485035 | 1484787 | | |
| NMTN-08971 | SAV1404 | | 1486074 | 1486343 | 1486135 | + | conserved hypothetical protein |
| NMTN-09592 | SAV1405 | | 1486368 | 1486991 | 1486928 | + | hypothetical protein |
| NMTN-09088 | SAV1407 | braB | 1488232 | 1489575 | 1488485 | − | branched-chain amino acid carrier |
| | | | | | | | protein |
| NMTN-03826 | SAV1407 | braB | 1488232 | 1489575 | 1488507 | | |
| NMTN-06490 | SAV1407 | braB | 1488232 | 1489575 | 1488507 | | |
| NMTN-06214 | SAV1407 | braB | 1488232 | 1489575 | 1488847 | | |
| NMTN-10208 | SAV1407 | braB | 1488232 | 1489575 | 1488852 | | |
| NMTN-02848 | SAV1407 | braB | 1488232 | 1489575 | 1489245 | | |
| NMTN-00420 | SAV1407 | braB | 1488232 | 1489575 | 1489313 | | |
| NMTN-10018 | SAV1407 | braB | 1488232 | 1489575 | 1489381 | | |
| NMTN-06295 | SAV1408 | | 1489801 | 1491687 | 1490385 | − | conserved hypothetical protein |
| NMTN-03005 | SAV1408 | | 1489801 | 1491687 | 1491156 | | |
| NMTN-11113 | SAV1408 | | 1489801 | 1491687 | 1491168 | | |
| NMTN-11700 | SAV1408 | | 1489801 | 1491687 | 1491271 | | |
| NMTN-06662 | SAV1408 | | 1489801 | 1491687 | 1491278 | | |
| NMTN-03580 | SAV1408 | | 1489801 | 1491687 | 1491465 | | |
| NMTN-03792 | SAV1411 | | 1492905 | 1493714 | 1493695 | − | ABC transporter homolog |
| NMTN-07930 | SAV1412 | odhB | 1494294 | 1495562 | 1494578 | − | dihydrolipoamide succinyltransferase |
| NMTN-04792 | SAV1412 | odhB | 1494294 | 1495562 | 1494677 | | |
| NMTN-01494 | SAV1412 | odhB | 1494294 | 1495562 | 1494717 | | |
| NMTN-13678 | SAV1412 | odhB | 1494294 | 1495562 | 1495516 | | |
| NMTN-07888 | SAV1413 | odhA | 1495599 | 1498373 | 1495674 | − | oxoglutarate dehydrogenase |
| NMTN-09416 | SAV1413 | odhA | 1495599 | 1498373 | 1495692 | | |
| NMTN-10823 | SAV1413 | odhA | 1495599 | 1498373 | 1495711 | | |
| NMTN-03753 | SAV1413 | odhA | 1495599 | 1498373 | 1495725 | | |
| NMTN-13413 | SAV1413 | odhA | 1495599 | 1498373 | 1496214 | | |
| NMTN-06555 | SAV1413 | odhA | 1495599 | 1498373 | 1496554 | | |
| NMTN-07266 | SAV1413 | odhA | 1495599 | 1498373 | 1496863 | | |
| NMTN-02026 | SAV1413 | odhA | 1495599 | 1498373 | 1497020 | | |
| NMTN-01754 | SAV1413 | odhA | 1495599 | 1498373 | 1497408 | | |
| NMTN-10214 | SAV1413 | odhA | 1495599 | 1498373 | 1498069 | | |
| NMTN-13248 | SAV1413 | odhA | 1495599 | 1498373 | 1498073 | | |
| NMTN-13411 | SAV1413 | odhA | 1495599 | 1498373 | 1498076 | | |
| NMTN-06603 | SAV1413 | odhA | 1495599 | 1498373 | 1498120 | | |
| NMTN-10023 | SAV1413 | odhA | 1495599 | 1498373 | 1498206 | | |
| NMTN-13148 | SAV1413 | odhA | 1495599 | 1498373 | 1498319 | | |
| NMTN-04120 | SAV1416 | | 1501139 | 1501339 | 1501262 | − | hypothetical protein |
| NMTN-12235 | SAV1417 | | 1501456 | 1502070 | 1501496 | − | conserved hypothetical protein |
| NMTN-12959 | SAV1420 | ctpA | 1504078 | 1505568 | 1504331 | − | probable carboxy-terminal processing |
| | | | | | | | proteinase |
| NMTN-10848 | SAV1420 | ctpA | 1504078 | 1505568 | 1504371 | | |
| NMTN-08261 | SAV1420 | ctpA | 1504078 | 1505568 | 1504470 | | |
| NMTN-09098 | SAV1420 | ctpA | 1504078 | 1505568 | 1504613 | | |
| NMTN-09785 | SAV1420 | ctpA | 1504078 | 1505568 | 1504678 | | |
| NMTN-13749 | SAV1420 | ctpA | 1504078 | 1505568 | 1504891 | | |
| NMTN-09941 | SAV1420 | ctpA | 1504078 | 1505568 | 1505031 | | |
| NMTN-10595 | SAV1423 | | 1506493 | 1506921 | 1506801 | − | conserved hypothetical protein |
| NMTN-02406 | SAV1423 | | 1506493 | 1506921 | 1506888 | | |
| NMTN-03609 | SAV1425 | | 1507533 | 1508372 | 1507594 | − | conserved hypothetical protein |
| NMTN-03610 | SAV1425 | | 1507533 | 1508372 | 1507594 | | |
| NMTN-12595 | SAV1425 | | 1507533 | 1508372 | 1507897 | | |
| NMTN-12679 | SAV1425 | | 1507533 | 1508372 | 1508096 | | |
| NMTN-11371 | SAV1425 | | 1507533 | 1508372 | 1508239 | | |
| NMTN-12821 | SAV1425 | | 1507533 | 1508372 | 1508239 | | |
| NMTN-12822 | SAV1425 | | 1507533 | 1508372 | 1508239 | | |
| NMTN-05309 | SAV1425 | | 1507533 | 1508372 | 1508278 | | |
| NMTN-05317 | SAV1429 | | 1510899 | 1512023 | 1511111 | − | conserved hypothetical protein |
| NMTN-11431 | SAV1429 | | 1510899 | 1512023 | 1511959 | | |
| NMTN-05515 | SAV1429 | | 1510899 | 1512023 | 1512007 | | |
| NMTN-02155 | SAV1432 | | 1512763 | 1513467 | 1512788 | + | conserved hypothetical protein |
| NMTN-07038 | SAV1432 | | 1512763 | 1513467 | 1512821 | | |
| NMTN-03810 | SAV1432 | | 1512763 | 1513467 | 1512824 | | |
| NMTN-13643 | SAV1432 | | 1512763 | 1513467 | 1512979 | | |
| NMTN-11448 | SAV1433 | | 1513950 | 1514351 | 1514095 | + | hypothetical protein |
| NMTN-10593 | SAV1433 | | 1513950 | 1514351 | 1514101 | | |
| NMTN-09463 | SAV1434 | ebhA | 1514410 | 1534551 | 1515246 | − | hypothetical protein |
| NMTN-05505 | SAV1434 | ebhA | 1514410 | 1534551 | 1515608 | | |
| NMTN-13081 | SAV1434 | ebhA | 1514410 | 1534551 | 1516190 | | |
| NMTN-00579 | SAV1434 | ebhA | 1514410 | 1534551 | 1517535 | | |
| NMTN-09418 | SAV1434 | ebhA | 1514410 | 1534551 | 1517981 | | |
| NMTN-02432 | SAV1434 | ebhA | 1514410 | 1534551 | 1518602 | | |
| NMTN-01690 | SAV1434 | ebhA | 1514410 | 1534551 | 1518897 | | |
| NMTN-07924 | SAV1434 | ebhA | 1514410 | 1534551 | 1519416 | | |
| NMTN-01692 | SAV1434 | ebhA | 1514410 | 1534551 | 1520745 | | |
| NMTN-01726 | SAV1434 | ebhA | 1514410 | 1534551 | 1520745 | | |
| NMTN-06873 | SAV1434 | ebhA | 1514410 | 1534551 | 1520745 | | |
| NMTN-11210 | SAV1434 | ebhA | 1514410 | 1534551 | 1520802 | | |
| NMTN-01731 | SAV1434 | ebhA | 1514410 | 1534551 | 1520887 | | |
| NMTN-06642 | SAV1434 | ebhA | 1514410 | 1534551 | 1521051 | | |
| NMTN-03287 | SAV1434 | ebhA | 1514410 | 1534551 | 1521566 | | |
| NMTN-14060 | SAV1434 | ebhA | 1514410 | 1534551 | 1521569 | | |
| NMTN-09629 | SAV1434 | ebhA | 1514410 | 1534551 | 1522151 | | |
| NMTN-06508 | SAV1434 | ebhA | 1514410 | 1534551 | 1522456 | | |
| NMTN-12072 | SAV1434 | ebhA | 1514410 | 1534551 | 1522655 | | |
| NMTN-07393 | SAV1434 | ebhA | 1514410 | 1534551 | 1522965 | | |
| NMTN-11977 | SAV1434 | ebhA | 1514410 | 1534551 | 1524921 | | |
| NMTN-10974 | SAV1434 | ebhA | 1514410 | 1534551 | 1526318 | | |
| NMTN-03812 | SAV1434 | ebhA | 1514410 | 1534551 | 1526525 | | |
| NMTN-12449 | SAV1434 | ebhA | 1514410 | 1534551 | 1526604 | | |
| NMTN-00580 | SAV1434 | ebhA | 1514410 | 1534551 | 1527487 | | |
| NMTN-12003 | SAV1434 | ebhA | 1514410 | 1534551 | 1528829 | | |
| NMTN-04942 | SAV1434 | ebhA | 1514410 | 1534551 | 1528890 | | |
| NMTN-10853 | SAV1434 | ebhA | 1514410 | 1534551 | 1529545 | | |
| NMTN-15257 | SAV1434 | ebhA | 1514410 | 1534551 | 1529545 | | |
| NMTN-02585 | SAV1434 | ebhA | 1514410 | 1534551 | 1531370 | | |
| NMTN-09796 | SAV1434 | ebhA | 1514410 | 1534551 | 1531490 | | |
| NMTN-04384 | SAV1434 | ebhA | 1514410 | 1534551 | 1531514 | | |
| NMTN-12813 | SAV1434 | ebhA | 1514410 | 1534551 | 1531514 | | |
| NMTN-06753 | SAV1434 | ebhA | 1514410 | 1534551 | 1531983 | | |
| NMTN-09156 | SAV1434 | ebhA | 1514410 | 1534551 | 1531986 | | |
| NMTN-10992 | SAV1434 | ebhA | 1514410 | 1534551 | 1532146 | | |
| NMTN-09582 | SAV1434 | ebhA | 1514410 | 1534551 | 1532509 | | |
| NMTN-12214 | SAV1434 | ebhA | 1514410 | 1534551 | 1532521 | | |
| NMTN-09506 | SAV1434 | ebhA | 1514410 | 1534551 | 1533133 | | |
| NMTN-06747 | SAV1434 | ebhA | 1514410 | 1534551 | 1533537 | | |
| NMTN-03696 | SAV1434 | ebhA | 1514410 | 1534551 | 1533620 | | |
| NMTN-07867 | SAV1434 | ebhA | 1514410 | 1534551 | 1534531 | | |
| NMTN-00480 | SAV1435 | ebhB | 1534612 | 1546284 | 1535735 | − | hypothetical protein |
| NMTN-09542 | SAV1435 | ebhB | 1534612 | 1546284 | 1536446 | | |
| NMTN-01535 | SAV1435 | ebhB | 1534612 | 1546284 | 1536942 | | |
| NMTN-01563 | SAV1435 | ebhB | 1534612 | 1546284 | 1537286 | | |
| NMTN-04863 | SAV1435 | ebhB | 1534612 | 1546284 | 1537403 | | |
| NMTN-12200 | SAV1435 | ebhB | 1534612 | 1546284 | 1538025 | | |
| NMTN-11589 | SAV1435 | ebhB | 1534612 | 1546284 | 1538466 | | |
| NMTN-05027 | SAV1435 | ebhB | 1534612 | 1546284 | 1538549 | | |
| NMTN-08732 | SAV1435 | ebhB | 1534612 | 1546284 | 1538663 | | |
| NMTN-13754d | SAV1435 | ebhB | 1534612 | 1546284 | 1538876 | | |
| NMTN-01576 | SAV1435 | ebhB | 1534612 | 1546284 | 1539059 | | |
| NMTN-03094 | SAV1435 | ebhB | 1534612 | 1546284 | 1539948 | | |
| NMTN-13847 | SAV1435 | ebhB | 1534612 | 1546284 | 1540175 | | |
| NMTN-01950 | SAV1435 | ebhB | 1534612 | 1546284 | 1540265 | | |
| NMTN-10047 | SAV1435 | ebhB | 1534612 | 1546284 | 1540676 | | |
| NMTN-10438 | SAV1435 | ebhB | 1534612 | 1546284 | 1540810 | | |
| NMTN-06312 | SAV1435 | ebhB | 1534612 | 1546284 | 1541409 | | |
| NMTN-09612 | SAV1435 | ebhB | 1534612 | 1546284 | 1541438 | | |
| NMTN-06354 | SAV1435 | ebhB | 1534612 | 1546284 | 1541928 | | |
| NMTN-14606 | SAV1435 | ebhB | 1534612 | 1546284 | 1541993 | | |
| NMTN-00288 | SAV1435 | ebhB | 1534612 | 1546284 | 1542480 | | |
| NMTN-09225 | SAV1435 | ebhB | 1534612 | 1546284 | 1542518 | | |
| NMTN-10645 | SAV1435 | ebhB | 1534612 | 1546284 | 1542905 | | |
| NMTN-06442 | SAV1435 | ebhB | 1534612 | 1546284 | 1543679 | | |
| NMTN-13013d | SAV1435 | ebhB | 1534612 | 1546284 | 1543788 | | |
| NMTN-09529 | SAV1435 | ebhB | 1534612 | 1546284 | 1543997 | | |
| NMTN-02417 | SAV1435 | ebhB | 1534612 | 1546284 | 1544112 | | |
| NMTN-13553 | SAV1435 | ebhB | 1534612 | 1546284 | 1544294 | | |
| NMTN-03648 | SAV1435 | ebhB | 1534612 | 1546284 | 1544635 | | |
| NMTN-09044 | SAV1435 | ebhB | 1534612 | 1546284 | 1544755 | | |
| NMTN-10238 | SAV1435 | ehbB | 1534612 | 1546284 | 1546264 | | |
| NMTN-13073 | SAV1436 | | 1546682 | 1548073 | 1546926 | − | Bit-like protein |
| NMTN-03505 | SAV1436 | | 1546682 | 1548073 | 1546978 | | |
| NMTN-07114 | SAV1436 | | 1546682 | 1548073 | 1548061 | | |
| NMTN-07769 | SAV1437 | | 1548229 | 1549551 | 1548638 | − | hypothetical protein |
| NMTN-15210 | SAV1437 | | 1548229 | 1549551 | 1548656 | | |
| NMTN-02311 | SAV1437 | | 1548229 | 1549551 | 1548658 | | |
| NMTN-10391 | SAV1437 | | 1548229 | 1549551 | 1548907 | | |
| NMTN-09822 | SAV1437 | | 1548229 | 1549551 | 1549117 | | |
| NMTN-03573 | SAV1438 | | 1549582 | 1550622 | 1550492 | − | threonine deaminase llvA homolog |
| NMTN-05429 | SAV1439 | | 1550717 | 1551835 | 1551203 | − | alanine dehydrogenase |
| NMTN-01628 | SAV1439 | | 1550717 | 1551835 | 1551505 | | |
| NMTN-06311 | SAV1439 | | 1550717 | 1551835 | 1551590 | | |
| NMTN-10883 | SAV1440 | | 1552311 | 1553189 | 1552837 | − | hypothetical protein |
| NMTN-06178 | SAV1441 | | 1553209 | 1556649 | 1553442 | − | conserved hypothetical protein |
| NMTN-13587 | SAV1441 | | 1553209 | 1556649 | 1553449 | | |
| NMTN-06279 | SAV1441 | | 1553209 | 1556649 | 1554557 | | |
| NMTN-00813 | SAV1441 | | 1553209 | 1556649 | 1554660 | | |
| NMTN-14000 | SAV1441 | | 1553209 | 1556649 | 1555066 | | |
| NMTN-10960 | SAV1441 | | 1553209 | 1556649 | 1556183 | | |
| NMTN-04669 | SAV1441 | | 1553209 | 1556649 | 1556539 | | |
| NMTN-10919 | SAV1442 | | 1556953 | 1558293 | 1557350 | + | conserved hypothetical protein |
| NMTN-12248 | SAV1442 | | 1556953 | 1558293 | 1558039 | | |
| NMTN-13050 | SAV1442 | | 1556953 | 1558293 | 1558154 | | |
| NMTN-02819 | SAV1442 | | 1556953 | 1558293 | 1558171 | | |
| NMTN-05628 | SAV1443 | | 1558499 | 1558831 | 1558665 | − | hypothetical protein |
| NMTN-10463 | SAV1444 | | 1558915 | 1560060 | 1558939 | − | conserved hypothetical protein |
| NMTN-08469 | SAV1444 | | 1558915 | 1560060 | 1558959 | | |
| NMTN-13090 | SAV1448 | | 1561625 | 1561975 | 1561794 | − | conserved hypothetical protein |
| NMTN-02660 | SAV1448 | | 1561625 | 1561975 | 1561858 | | |
| NMTN-00061 | SAV1451 | | 1565887 | 1566228 | 1565924 | − | hypothetical protein |
| NMTN-08678 | SAV1451 | | 1565887 | 1566228 | 1566160 | | |
| NMTN-11116 | SAV1454 | asnS | 1567896 | 1569188 | 1568202 | − | asparaginyl-tRNA synthetase |
| NMTN-02119 | SAV1455 | dinG | 1569510 | 1572203 | 1569790 | − | probable ATP-dependent DNA helicase |
| NMTN-13660 | SAV1455 | dinG | 1569510 | 1572203 | 1570043 | | |
| NMTN-12299 | SAV1455 | dinG | 1569510 | 1572203 | 1570300 | | |
| NMTN-09305 | SAV1455 | dinG | 1569510 | 1572203 | 1570336 | | |
| NMTN-00788 | SAV1455 | dinG | 1569510 | 1572203 | 1570869 | | |
| NMTN-01522 | SAV1455 | dinG | 1569510 | 1572203 | 1570870 | | |
| NMTN-01523 | SAV1455 | dinG | 1569510 | 1572203 | 1570870 | | |
| NMTN-01524 | SAV1455 | dinG | 1569510 | 1572203 | 1570870 | | |
| NMTN-01526 | SAV1455 | dinG | 1569510 | 1572203 | 1570870 | | |
| NMTN-01527 | SAV1455 | dinG | 1569510 | 1572203 | 1570870 | | |
| NMTN-01538 | SAV1455 | dinG | 1569510 | 1572203 | 1570870 | | |
| NMTN-01539 | SAV1455 | dinG | 1569510 | 1572203 | 1570870 | | |
| NMTN-01540 | SAV1455 | dinG | 1569510 | 1572203 | 1570870 | | |
| NMTN-05585 | SAV1455 | dinG | 1569510 | 1572203 | 1570870 | | |
| NMTN-01561 | SAV1455 | dinG | 1569510 | 1572203 | 1570919 | | |
| NMTN-11510 | SAV1455 | dinG | 1569510 | 1572203 | 1571325 | | |
| NMTN-07319 | SAV1455 | dinG | 1569510 | 1572203 | 1571359 | | |
| NMTN-06258 | SAV1455 | dinG | 1569510 | 1572203 | 1571414 | | |
| NMTN-12772 | SAV1455 | dinG | 1569510 | 1572203 | 1571485 | | |
| NMTN-09834 | SAV1455 | dinG | 1569510 | 1572203 | 1571545 | | |
| NMTN-10432 | SAV1455 | dinG | 1569510 | 1572203 | 1572130 | | |
| NMTN-10521 | SAV1458 | | 1574392 | 1575534 | 1574778 | − | hypothetical protein |
| NMTN-14032 | SAV1459 | | 1575778 | 1576095 | 1575896 | − | conserved hypothetical protein |
| NMTN-12127 | SAV1461 | | 1577184 | 1577771 | 1577255 | − | conserved hypothetical protein |
| NMTN-11002 | SAV1461 | | 1577184 | 1577771 | 1577420 | | |
| NMTN-04132 | SAV1461 | | 1577184 | 1577771 | 1577450 | | |
| NMTN-12066 | SAV1461 | | 1577184 | 1577771 | 1577679 | | |
| NMTN-09151 | SAV1461 | | 1577184 | 1577771 | 1577698 | | |
| NMTN-09155 | SAV1461 | | 1577184 | 1577771 | 1577698 | | |
| NMTN-09165 | SAV1461 | | 1577184 | 1577771 | 1577698 | | |
| NMTN-09168 | SAV1461 | | 1577184 | 1577771 | 1577698 | | |
| NMTN-09173 | SAV1461 | | 1577184 | 1577771 | 1577698 | | |
| NMTN-09177 | SAV1461 | | 1577184 | 1577771 | 1577698 | | |
| NMTN-09179 | SAV1461 | | 1577184 | 1577771 | 1577698 | | |
| NMTN-09913 | SAV1463 | | 1578350 | 1579594 | 1579154 | − | conserved hypothetical protein |
| NMTN-01451 | SAV1463 | | 1578350 | 1579594 | 1579294 | | |
| NMTN-01457 | SAV1463 | | 1578350 | 1579594 | 1579294 | | |
| NMTN-01462 | SAV1463 | | 1578350 | 1579594 | 1579294 | | |
| NMTN-01463 | SAV1463 | | 1578350 | 1579594 | 1579294 | | |
| NMTN-01464 | SAV1463 | | 1578350 | 1579594 | 1579294 | | |
| NMTN-01465 | SAV1463 | | 1578350 | 1579594 | 1579294 | | |
| NMTN-01471 | SAV1463 | | 1578350 | 1579594 | 1579294 | | |
| NMTN-10524 | SAV1467 | | 1583551 | 1583751 | 1583763 | − | hypothetical protein |
| NMTN-00200 | SAV1469 | ndk | 1583960 | 1584409 | 1584298 | − | nucleoside diphosphate kinase |
| NMTN-12690 | SAV1476 | | 1590183 | 1591358 | 1590319 | − | 30S ribosomal protein S1 |
| NMTN-02484 | SAV1476 | | 1590183 | 1591358 | 1590452 | | |
| NMTN-09236 | SAV1476 | | 1590183 | 1591358 | 1591005 | | |
| NMTN-10445 | SAV1478 | cmk | 1592070 | 1592729 | 1592225 | − | cytidylate kinase |
| NMTN-15282 | SAV1480 | | 1593889 | 1594875 | 1594044 | − | hypothetical protein |
| NMTN-13173 | SAV1480 | | 1593889 | 1594875 | 1594143 | | |
| NMTN-11276 | SAV1480 | | 1593889 | 1594875 | 1594323 | | |
| NMTN-00664 | SAV1480 | | 1593889 | 1594875 | 1594492 | | |
| NMTN-02088 | SAV1480 | | 1593889 | 1594875 | 1594547 | | |
| NMTN-07946 | SAV1480 | | 1593889 | 1594875 | 1594596 | | |
| NMTN-01733 | SAV1482 | | 1596890 | 1598269 | 1597122 | − | probable ATP-dependent DNA helicase |
| | | | | | | | RecQ |
| NMTN-14600 | SAV1482 | | 1596890 | 1598269 | 1597246 | | |
| NMTN-08645 | SAV1482 | | 1596890 | 1598269 | 1598080 | | |
| NMTN-11560 | SAV1482 | | 1596890 | 1598269 | 1598080 | | |
| NMTN-11710 | SAV1482 | | 1596890 | 1598269 | 1598080 | | |
| NMTN-09531 | SAV1483 | | 1598259 | 1599212 | 1598687 | − | conserved hypothetical protein |
| NMTN-04523 | SAV1483 | | 1598259 | 1599212 | 1599165 | | |
| NMTN-04516 | SAV1483 | | 1598259 | 1599212 | 1599171 | | |
| NMTN-04755 | SAV1483 | | 1598259 | 1599212 | 1599171 | | |
| NMTN-04911 | SAV1483 | | 1598259 | 1599212 | 1599171 | | |
| NMTN-11752 | SAV1484 | fer | 1599319 | 1599567 | 1599404 | + | ferredoxin |
| NMTN-09828 | SAV1484 | fer | 1599319 | 1599567 | 1599563 | | |
| NMTN-02826 | SAV1485 | | 1599673 | 1600218 | 1599704 | − | conserved hypothetical protein |
| NMTN-13673 | SAV1485 | | 1599673 | 1600218 | 1599902 | | |
| NMTN-00334 | SAV1485 | | 1599673 | 1600218 | 1600184 | | |
| NMTN-00347 | SAV1485 | | 1599673 | 1600218 | 1600184 | | |
| NMTN-09669 | SAV1488 | | 1602999 | 1603904 | 1603740 | − | hypothetical protein |
| NMTN-10961 | SAV1489 | | 1603994 | 1606156 | 1604361 | − | hypothetical protein |
| NMTN-13535 | SAV1489 | | 1603994 | 1606156 | 1604507 | | |
| NMTN-12761 | SAV1489 | | 1603994 | 1606156 | 1604531 | | |
| NMTN-06275 | SAV1489 | | 1603994 | 1606156 | 1604844 | | |
| NMTN-08496 | SAV1489 | | 1603994 | 1606156 | 1605077 | | |
| NMTN-08794 | SAV1489 | | 1603994 | 1606156 | 1606159 | | |
| NMTN-12844 | SAV1491 | srrB | 1607319 | 1609085 | 1608752 | − | staphylococcal respiratory response |
| | | | | | | | protein |
| NMTN-00634 | SAV1493 | rluB | 1609909 | 1610646 | 1610090 | − | ribosomal large subunit pseudouridine |
| | | | | | | | synthase B |
| NMTN-08902 | SAV1494 | | 1610639 | 1611181 | 1610941 | − | conserved hypothetical protein |
| NMTN-01764 | SAV1495 | | 1611174 | 1611929 | 1611789 | − | conserved hypothetical protein |
| NMTN-11634 | SAV1495 | | 1611174 | 1611929 | 1611814 | | |
| NMTN-02742 | SAV1495 | | 1611174 | 1611929 | 1611900 | | |
| NMTN-10457 | SAV1496 | | 1611997 | 1612503 | 1612021 | + | conserved hypothetical protein |
| NMTN-03765 | SAV1499 | | 1614071 | 1614613 | 1614287 | − | conserved hypothetical protein |
| NMTN-08669 | SAV1500 | | 1614695 | 1615603 | 1614854 | + | conserved hypothetical protein |
| NMTN-03270 | SAV1501 | | 1615819 | 1616067 | 1615832 | − | hypothetical protein |
| NMTN-04692 | SAV1501 | | 1615819 | 1616067 | 1615938 | | |
| NMTN-09635 | SAV1502 | | 1616083 | 1616838 | 1616213 | − | hypothetical protein |
| NMTN-02184 | SAV1502 | | 1616083 | 1616838 | 1616216 | | |
| NMTN-12283 | SAV1502 | | 1616083 | 1616838 | 1616461 | | |
| NMTN-03207 | SAV1502 | | 1616083 | 1616838 | 1616687 | | |
| NMTN-03208 | SAV1502 | | 1616083 | 1616838 | 1616687 | | |
| NMTN-03210 | SAV1502 | | 1616083 | 1616838 | 1616687 | | |
| NMTN-03211 | SAV1502 | | 1616083 | 1616838 | 1616687 | | |
| NMTN-03212 | SAV1502 | | 1616083 | 1616838 | 1616687 | | |
| NMTN-03219 | SAV1502 | | 1616083 | 1616838 | 1616687 | | |
| NMTN-14594 | SAV1503 | | 1616980 | 1617795 | 1617287 | + | hypothetical protein |
| NMTN-12154 | SAV1503 | | 1616980 | 1617795 | 1617596 | | |
| NMTN-14689 | SAV1503 | | 1616980 | 1617795 | 1617714 | | |
| NMTN-14690 | SAV1503 | | 1616980 | 1617795 | 1617714 | | |
| NMTN-14696 | SAV1503 | | 1616980 | 1617795 | 1617714 | | |
| NMTN-14697 | SAV1503 | | 1616980 | 1617795 | 1617714 | | |
| NMTN-14698 | SAV1503 | | 1616980 | 1617795 | 1617714 | | |
| NMTN-14749 | SAV1503 | | 1616980 | 1617795 | 1617714 | | |
| NMTN-14762 | SAV1503 | | 1616980 | 1617795 | 1617714 | | |
| NMTN-14764 | SAV1503 | | 1616980 | 1617795 | 1617714 | | |
| NMTN-14768 | SAV1503 | | 1616980 | 1617795 | 1617714 | | |
| NMTN-14771 | SAV1503 | | 1616980 | 1617795 | 1617714 | | |
| NMTN-14773 | SAV1503 | | 1616980 | 1617795 | 1617714 | | |
| NMTN-09842 | SAV1503 | | 1616980 | 1617795 | 1617783 | | |
| NMTN-03915 | SAV1506 | | 1620845 | 1621711 | 1621146 | + | transcription regulator AraC/XylS family |
| | | | | | | | homolog |
| NMTN-10195 | SAV1507 | malA | 1621793 | 1623442 | 1621849 | − | alpha-D-1,4-glucosidase |
| NMTN-10496 | SAV1507 | malA | 1621793 | 1623442 | 1623338 | | |
| NMTN-14031 | SAV1508 | malR | 1623458 | 1624477 | 1623688 | − | maltose operon transcriptional repressor |
| NMTN-05490 | SAV1508 | malR | 1623458 | 1624477 | 1623768 | | |
| NMTN-04920 | SAV1508 | malR | 1623458 | 1624477 | 1624009 | | |
| NMTN-13798 | SAV1508 | malR | 1623458 | 1624477 | 1624137 | | |
| NMTN-06273 | SAV1508 | malR | 1623458 | 1624477 | 1624299 | | |
| NMTN-07692 | SAV1508 | malR | 1624671 | 1625042 | 1624691 | | |
| NMTN-07430 | SAV1509 | | 1624671 | 1625042 | 1624727 | − | conserved hypothetical protein |
| NMTN-00545 | SAV1509 | | 1624671 | 1625042 | 1624742 | | |
| NMTN-01616 | SAV1509 | | 1624671 | 1625042 | 1625016 | | |
| NMTN-06529 | SAV1511 | gnd | 1627042 | 1628448 | 1627036 | − | phosphogluconate dehydrogenase |
| NMTN-10611 | SAV1512 | | 1628516 | 1629649 | 1629153 | − | hypothetical protein |
| NMTN-00153 | SAV1512 | | 1628516 | 1629649 | 1629560 | | |
| NMTN-10518 | SAV1513 | | 1630218 | 1631198 | 1630219 | − | conserved hypothetical protein |
| NMTN-10029 | SAV1513 | | 1630218 | 1631198 | 1630230 | | |
| NMTN-05030 | SAV1513 | | 1630218 | 1631198 | 1630736 | | |
| NMTN-09889 | SAV1513 | | 1630218 | 1631198 | 1630840 | | |
| NMTN-10081 | SAV1513 | | 1630218 | 1631198 | 1630942 | | |
| NMTN-09950 | SAV1514 | | 1631212 | 1631649 | 1631259 | − | conserved hypothetical protein |
| NMTN-09564 | SAV1515 | bmfBB | 1632000 | 1633274 | 1632589 | − | branched-chain alpha-keto acid |
| | | | | | | | dehydrogenase E2 |
| NMTN-10858 | SAV1515 | bmfBB | 1632000 | 1633274 | 1633041 | | |
| NMTN-13527 | SAV1516 | bfmBAB | 1633287 | 1634270 | 1633995 | − | branched-chain alpha-keto acid |
| | | | | | | | dehydrogenase E1 |
| NMTN-13468 | SAV1517 | bfmBAA | 1634270 | 1635262 | 1634864 | − | branched-chain alpha-keto acid |
| | | | | | | | dehydrogenase E1 |
| NMTN-13873 | SAV1517 | bfmBAA | 1634270 | 1635262 | 1635029 | | |
| NMTN-08777 | SAV1518 | | 1635278 | 1636699 | 1635528 | − | dihydrolipoamide dehydrogenase |
| NMTN-12287 | SAV1518 | | 1635278 | 1636699 | 1635684 | | |
| NMTN-10390 | SAV1518 | | 1635278 | 1636699 | 1635900 | | |
| NMTN-09439 | SAV1519 | recN | 1636850 | 1638529 | 1638296 | − | DNA repair protein |
| NMTN-02320 | SAV1520 | ahrC | 1638545 | 1638997 | 1638618 | − | arginine repressor |
| NMTN-02610 | SAV1520 | ahrC | 1638545 | 1638997 | 1638692 | | |
| NMTN-11035 | SAV1520 | ahrC | 1638545 | 1638997 | 1638736 | | |
| NMTN-00598 | SAV1520 | ahrC | 1638545 | 1638997 | 1638835 | | |
| NMTN-08966 | SAV1521 | ispA | 1639429 | 1640310 | 1639526 | − | geranyltranstransferase homolog |
| NMTN-03133 | SAV1521 | ispA | 1639429 | 1640310 | 1639694 | | |
| NMTN-10447 | SAV1521 | ispA | 1639429 | 1640310 | 1639924 | | |
| NMTN-08565 | SAV1521 | ispA | 1639429 | 1640310 | 1639991 | | |
| NMTN-00218 | SAV1521 | ispA | 1639429 | 1640310 | 1640007 | | |
| NMTN-13659 | SAV1521 | ispA | 1639429 | 1640310 | 1640210 | | |
| NMTN-04349 | SAV1523 | | 1640511 | 1641848 | 1640552 | − | hypothetical protein |
| NMTN-14596 | SAV1523 | | 1640511 | 1641848 | 1640915 | | |
| NMTN-08618 | SAV1523 | | 1640511 | 1641848 | 1640954 | | |
| NMTN-08432 | SAV1523 | | 1640511 | 1641848 | 1640966 | | |
| NMTN-14396 | SAV1523 | | 1640511 | 1641848 | 1641025 | | |
| NMTN-14694d | SAV1523 | | 1640511 | 1641848 | 1641304 | | |
| NMTN-08782 | SAV1523 | | 1640511 | 1641848 | 1641377 | | |
| NMTN-01828 | SAV1523 | | 1640511 | 1641848 | 1641527 | | |
| NMTN-01978 | SAV1523 | | 1640511 | 1641848 | 1641536 | | |
| NMTN-12092 | SAV1523 | | 1640511 | 1641848 | 1641802 | | |
| NMTN-04651 | SAV1523 | | 1640511 | 1641848 | 1641849 | | |
| NMTN-13764 | SAV1524 | | 1641865 | 1642254 | 1641991 | − | hypothetical protein |
| NMTN-14011 | SAV1524 | | 1641865 | 1642254 | 1641994 | | |
| NMTN-08273 | SAV1524 | | 1641865 | 1642254 | 1642064 | | |
| NMTN-08274 | SAV1524 | | 1641865 | 1642254 | 1642064 | | |
| NMTN-08278 | SAV1524 | | 1641865 | 1642254 | 1642064 | | |
| NMTN-09368 | SAV1529 | | 1645686 | 1645997 | 1645958 | − | Xaa-Pro dipeptidase |
| NMTN-12331 | SAV1530 | | 1646053 | 1646628 | 1646193 | − | Xaa-Pro dipeptidase |
| NMTN-10040 | SAV1530 | | 1646053 | 1646628 | 1646376 | | |
| NMTN-12974 | SAV1535 | | 1649349 | 1650821 | 1649697 | − | glycine dehydrogenase subunit 2 |
| | | | | | | | homologue |
| NMTN-02082 | SAV1535 | | 1649349 | 1650821 | 1649881 | | |
| NMTN-09577 | SAV1535 | | 1649349 | 1650821 | 1650265 | | |
| NMTN-06418 | SAV1535 | | 1649349 | 1650821 | 1650768 | | |
| NMTN-10762 | SAV1536 | | 1650814 | 1652160 | 1650888 | − | glycine dehydrogenase subunit 1 |
| NMTN-12213 | SAV1536 | | 1650814 | 1652160 | 1650951 | | |
| NMTN-02182 | SAV1536 | | 1650814 | 1652160 | 1651019 | | |
| NMTN-11197 | SAV1536 | | 1650814 | 1652160 | 1651440 | | |
| NMTN-13786 | SAV1536 | | 1650814 | 1652160 | 1651636 | | |
| NMTN-01569 | SAV1536 | | 1650814 | 1652160 | 1651650 | | |
| NMTN-11615 | SAV1537 | | 1652180 | 1653271 | 1652626 | − | aminomethyltransferase |
| NMTN-02594 | SAV1537 | | 1652180 | 1653271 | 1653241 | | |
| NMTN-02470 | SAV1539 | | 1654187 | 1654684 | 1654257 | − | hypothetical protein |
| NMTN-04366 | SAV1539 | | 1654187 | 1654684 | 1654423 | | |
| NMTN-09937 | SAV1540 | | 1654602 | 1654901 | 1654890 | − | hypothetical protein |
| NMTN-09268 | SAV1541 | | 1654888 | 1655322 | 1654935 | − | hypothetical protein |
| NMTN-09270 | SAV1541 | | 1654888 | 1655322 | 1654935 | | |
| NMTN-07768 | SAV1541 | | 1654888 | 1655322 | 1655218 | | |
| NMTN-10049 | SAV1542 | | 1655312 | 1655623 | 1655460 | − | exogenous DNA-binding protein comGC |
| NMTN-11174 | SAV1542 | | 1655312 | 1655623 | 1655569 | | |
| NMTN-06550 | SAV1543 | | 1655637 | 1656707 | 1655793 | − | hypothetical protein |
| NMTN-03679 | SAV1543 | | 1655637 | 1656707 | 1655852 | | |
| NMTN-06373 | SAV1543 | | 1655637 | 1656707 | 1656182 | | |
| NMTN-10067 | SAV1543 | | 1655637 | 1656707 | 1656406 | | |
| NMTN-04447 | SAV1544 | | 1656679 | 1657653 | 1657002 | − | hypothetical protein |
| NMTN-14616 | SAV1544 | | 1656679 | 1657653 | 1657260 | | |
| NMTN-10440 | SAV1544 | | 1656679 | 1657653 | 1657484 | | |
| NMTN-08997 | SAV1544 | | 1656679 | 1657653 | 1657562 | | |
| NMTN-05124 | SAV1545 | | 1657705 | 1658328 | 1657912 | − | conserved hypothetical protein |
| NMTN-14429 | SAV1545 | | 1657705 | 1658328 | 1657912 | | |
| NMTN-10927 | SAV1546 | | 1658325 | 1658654 | 1658574 | − | conserved hypothetical protein |
| NMTN-13005 | SAV1547 | glcK | 1658654 | 1659640 | 1658868 | − | glucokinase |
| NMTN-01495 | SAV1547 | glcK | 1658654 | 1659640 | 1658900 | | |
| NMTN-08539 | SAV1547 | glcK | 1658654 | 1659640 | 1659006 | | |
| NMTN-08650 | SAV1547 | glcK | 1658654 | 1659640 | 1659286 | | |
| NMTN-13800 | SAV1549 | | 1659821 | 1661284 | 1660632 | − | conserved hypothetical protein |
| NMTN-08948 | SAV1549 | | 1659821 | 1661284 | 1660962 | | |
| NMTN-15172 | SAV1549 | | 1659821 | 1661284 | 1660963 | | |
| NMTN-09413 | SAV1549 | | 1659821 | 1661284 | 1660981 | | |
| NMTN-09080 | SAV1549 | | 1659821 | 1661284 | 1661009 | | |
| NMTN-00130 | SAV1550 | | 1661296 | 1661835 | 1661310 | − | conserved hypothetical protein |
| NMTN-11029 | SAV1555 | | 1665774 | 1666637 | 1666171 | − | hypothetical protein |
| NMTN-01591 | SAV1556 | | 1666680 | 1667447 | 1666964 | − | hypothetical protein |
| NMTN-00845 | SAV1556 | | 1666680 | 1667447 | 1667211 | | |
| NMTN-01864 | SAV1556 | | 1666680 | 1667447 | 1667211 | | |
| NMTN-09048 | SAV1556 | | 1666680 | 1667447 | 1667216 | | |
| NMTN-10520 | SAV1557 | | 1667591 | 1668481 | 1667740 | − | hypothetical protein |
| NMTN-05095 | SAV1557 | | 1667591 | 1668481 | 1668337 | | |
| NMTN-13559 | SAV1558 | | 1668491 | 1669837 | 1668496 | − | hypothetical protein |
| NMTN-10627 | SAV1558 | | 1668491 | 1669837 | 1669362 | | |
| NMTN-11890 | SAV1558 | | 1668491 | 1669837 | 1669408 | | |
| NMTN-04886 | SAV1563 | | 1675070 | 1675888 | 1675184 | − | conserved hypothetical protein |
| NMTN-12549 | SAV1563 | | 1675070 | 1675888 | 1675243 | | |
| NMTN-03872 | SAV1563 | | 1675070 | 1675888 | 1675323 | | |
| NMTN-03904 | SAV1563 | | 1675070 | 1675888 | 1675328 | | |
| NMTN-10421 | SAV1563 | | 1675070 | 1675888 | 1675485 | | |
| NMTN-12526 | SAV1563 | | 1675070 | 1675888 | 1675662 | | |
| NMTN-03909 | SAV1563 | | 1675070 | 1675888 | 1675788 | | |
| NMTN-10510 | SAV1563 | | 1675070 | 1675888 | 1675854 | | |
| NMTN-10839 | SAV1571 | phoH | 1681302 | 1682249 | 1681677 | − | phosphate starvation-induced protein |
| | | | | | | | phoH homologue |
| NMTN-08889 | SAV1571 | phoH | 1681302 | 1682249 | 1682234 | | |
| NMTN-07544 | SAV1572 | | 1682553 | 1683251 | 1682692 | − | conserved hypothetical protein |
| NMTN-09146 | SAV1572 | | 1682553 | 1683251 | 1682800 | | |
| NMTN-06393 | SAV1572 | | 1682553 | 1683251 | 1682953 | | |
| NMTN-11862 | SAV1573 | | 1683268 | 1684257 | 1683687 | − | conserved hypothetical protein |
| NMTN-06402 | SAV1573 | | 1683268 | 1684257 | 1684101 | | |
| NMTN-07983 | SAV1573 | | 1683268 | 1684257 | 1684215 | | |
| NMTN-08686 | SAV1573 | | 1683268 | 1684257 | 1684253 | | |
| NMTN-10437 | SAV1574 | | 1684275 | 1684982 | 1684318 | − | conserved hypothetical protein |
| NMTN-10954 | SAV1574 | | 1684275 | 1684982 | 1684336 | | |
| NMTN-10271 | SAV1574 | | 1684275 | 1684982 | 1684355 | | |
| NMTN-15190 | SAV1574 | | 1684275 | 1684982 | 1684373 | | |
| NMTN-11310 | SAV1574 | | 1684275 | 1684982 | 1684550 | | |
| NMTN-09721 | SAV1574 | | 1684275 | 1684982 | 1684617 | | |
| NMTN-05383 | SAV1574 | | 1684275 | 1684982 | 1684639 | | |
| NMTN-10399 | SAV1576 | | 1685671 | 1687017 | 1685740 | − | conserved hypothetical protein |
| NMTN-05130 | SAV1576 | | 1685671 | 1687017 | 1687016 | | |
| NMTN-10031 | SAV1577 | | 1687024 | 1687776 | 1687551 | − | conserved hypothetical protein |
| NMTN-00034 | SAV1577 | | 1687024 | 1687776 | 1687700 | | |
| NMTN-09459 | SAV1583 | hemN | 1693631 | 1694755 | 1694092 | − | oxygen-independent |
| | | | | | | | coproporphyrinogen oxidase III |
| NMTN-09074 | SAV1583 | hemN | 1693631 | 1694755 | 1694647 | | |
| NMTN-00481 | SAV1583 | hemN | 1693631 | 1694755 | 1694736 | | |
| NMTN-15237 | SAV1585 | lepA | 1695321 | 1697144 | 1695827 | − | GTP-binding protein |
| NMTN-09748 | SAV1588 | | 1698817 | 1701018 | 1699136 | − | hypothetical protein |
| NMTN-01942 | SAV1588 | | 1698817 | 1701018 | 1699334 | | |
| NMTN-13467 | SAV1588 | | 1698817 | 1701018 | 1699334 | | |
| NMTN-04721 | SAV1588 | | 1698817 | 1701018 | 1699356 | | |
| NMTN-11840 | SAV1588 | | 1698817 | 1701018 | 1699428 | | |
| NMTN-13582 | SAV1588 | | 1698817 | 1701018 | 1699501 | | |
| NMTN-10072 | SAV1588 | | 1698817 | 1701018 | 1699617 | | |
| NMTN-03153 | SAV1588 | | 1698817 | 1701018 | 1700079 | | |
| NMTN-09905 | SAV1588 | | 1698817 | 1701018 | 1700578 | | |
| NMTN-02535 | SAV1588 | | 1698817 | 1701018 | 1700712 | | |
| NMTN-06704 | SAV1588 | | 1698817 | 1701018 | 1700827 | | |
| NMTN-15341 | SAV1589 | comEB | 1701023 | 1701484 | 1701067 | − | late competence operon required for |
| | | | | | | | DNA binding and uptake |
| NMTN-14510 | SAV1590 | | 1701576 | 1702253 | 1701752 | − | hypothetical protein |
| NMTN-01528 | SAV1590 | | 1701576 | 1702253 | 1701928 | | |
| NMTN-03623 | SAV1590 | | 1701576 | 1702253 | 1701955 | | |
| NMTN-04436 | SAV1590 | | 1701576 | 1702253 | 1702137 | | |
| NMTN-03417 | SAV1591 | | 1702302 | 1703018 | 1702523 | − | conserved hypothetical protein |
| NMTN-13165 | SAV1593 | | 1703375 | 1703959 | 1703417 | − | conserved hypothetical protein |
| NMTN-09804 | SAV1596 | aroE | 1704815 | 1705621 | 1705007 | − | shikimate dehydrogenease |
| NMTN-00573 | SAV1596 | aroE | 1704815 | 1705621 | 1705077 | | |
| NMTN-01788 | SAV1596 | aroE | 1704815 | 1705621 | 1705329 | | |
| NMTN-07513 | SAV1596 | aroE | 1704815 | 1705621 | 1705429 | | |
| NMTN-09942 | SAV1596 | aroE | 1704815 | 1705621 | 1705562 | | |
| NMTN-14659 | SAV1600 | | 1708283 | 1708552 | 1708463 | + | hypothetical protein |
| NMTN-10658 | SAV1601 | | 1708754 | 1708942 | 1708776 | − | enterotoxin homolog |
| NMTN-14387 | SAV1603 | | 1710271 | 1711491 | 1710425 | − | conserved hypothetical protein |
| NMTN-12817 | SAV1603 | | 1710271 | 1711491 | 1710691 | | |
| NMTN-10574 | SAV1603 | | 1710271 | 1711491 | 1710795 | | |
| NMTN-10488 | SAV1603 | | 1710271 | 1711491 | 1711001 | | |
| NMTN-05136 | SAV1603 | | 1710271 | 1711491 | 1711046 | | |
| NMTN-09087 | SAV1603 | | 1710271 | 1711491 | 1711377 | | |
| NMTN-04125 | SAV1604 | | 1711780 | 1713009 | 1712408 | − | conserved hypothetical protein |
| NMTN-09980 | SAV1604 | | 1711780 | 1713009 | 1712536 | | |
| NMTN-04677 | SAV1604 | | 1711780 | 1713009 | 1712586 | | |
| NMTN-10651 | SAV1604 | | 1711780 | 1713009 | 1712679 | | |
| NMTN-03405 | SAV1604 | | 1711780 | 1713009 | 1712796 | | |
| NMTN-10511 | SAV1604 | | 1711780 | 1713009 | 1712931 | | |
| NMTN-02099 | SAV1606 | | 1713774 | 1715135 | 1714634 | − | acetyl-CoA carboxylase |
| NMTN-00048 | SAV1606 | | 1713774 | 1715135 | 1714978 | | |
| NMTN-12009 | SAV1606 | | 1713774 | 1715135 | 1715012 | | |
| NMTN-02005 | SAV1606 | | 1713774 | 1715135 | 1715084 | | |
| NMTN-10243 | SAV1606 | | 1713774 | 1715135 | 1715097 | | |
| NMTN-00810 | SAV1607 | | 1715149 | 1715598 | 1715159 | − | hypothetical protein |
| NMTN-09590 | SAV1608 | | 1715600 | 1716610 | 1716166 | − | conserved hypothetical protein |
| NMTN-02412 | SAV1608 | | 1715600 | 1716610 | 1716208 | | |
| NMTN-02324 | SAV1608 | | 1715600 | 1716610 | 1716450 | | |
| NMTN-03667 | SAV1609 | | 1716600 | 1717334 | 1716726 | − | conserved hypothetical protein |
| NMTN-10608 | SAV1609 | | 1716600 | 1717334 | 1716839 | | |
| NMTN-06653 | SAV1609 | | 1716600 | 1717334 | 1716886 | | |
| NMTN-05512 | SAV1609 | | 1716600 | 1717334 | 1716945 | | |
| NMTN-06452 | SAV1609 | | 1716600 | 1717334 | 1716979 | | |
| NMTN-09775 | SAV1609 | | 1716600 | 1717334 | 1717028 | | |
| NMTN-09767 | SAV1609 | | 1716600 | 1717334 | 1717155 | | |
| NMTN-13426 | SAV1610 | greA | 1717660 | 1718136 | 1717751 | − | transcription elongation factor |
| NMTN-03914 | SAV1611 | udk | 1718164 | 1718787 | 1718324 | − | uridine kinase |
| NMTN-09647 | SAV1611 | udk | 1718164 | 1718787 | 1718447 | | |
| NMTN-13380 | SAV1611 | udk | 1718164 | 1718787 | 1718538 | | |
| NMTN-02786 | SAV1611 | udk | 1718164 | 1718787 | 1718682 | | |
| NMTN-10251 | SAV1612 | | 1718787 | 1720055 | 1719139 | − | hypothetical protein |
| NMTN-10375 | SAV1612 | | 1718787 | 1720055 | 1719139 | | |
| NMTN-13889 | SAV1612 | | 1718787 | 1720055 | 1719238 | | |
| NMTN-08658 | SAV1612 | | 1718787 | 1720055 | 1719475 | | |
| NMTN-07995 | SAV1612 | | 1718787 | 1720055 | 1719486 | | |
| NMTN-13271 | SAV1612 | | 1718787 | 1720055 | 1719645 | | |
| NMTN-11070 | SAV1612 | | 1718787 | 1720055 | 1719814 | | |
| NMTN-13394 | SAV1612 | | 1718787 | 1720055 | 1719956 | | |
| NMTN-07781 | SAV1612 | | 1718787 | 1720055 | 1720042 | | |
| NMTN-06599 | SAV1613 | | 1720067 | 1720990 | 1720085 | − | hypothetical protein |
| NMTN-06403 | SAV1613 | | 1720067 | 1720990 | 1720230 | | |
| NMTN-09960 | SAV1613 | | 1720067 | 1720990 | 1720307 | | |
| NMTN-09909 | SAV1613 | | 1720067 | 1720990 | 1720466 | | |
| NMTN-05586 | SAV1613 | | 1720067 | 1720990 | 1720816 | | |
| NMTN-08097 | SAV1614 | | 1720993 | 1721631 | 1721028 | − | hypothetical protein |
| NMTN-08257 | SAV1614 | | 1720993 | 1721631 | 1721046 | | |
| NMTN-08092 | SAV1614 | | 1720993 | 1721631 | 1721048 | | |
| NMTN-08271 | SAV1614 | | 1720993 | 1721631 | 1721048 | | |
| NMTN-08093 | SAV1614 | | 1720993 | 1721631 | 1721050 | | |
| NMTN-08099 | SAV1614 | | 1720993 | 1721631 | 1721050 | | |
| NMTN-02797 | SAV1614 | | 1720993 | 1721631 | 1721303 | | |
| NMTN-09933 | SAV1614 | | 1720993 | 1721631 | 1721608 | | |
| NMTN-13374 | SAV1616 | | 1722239 | 1722610 | 1722520 | − | conserved hypothetical protein |
| NMTN-13118 | SAV1617 | | 1722671 | 1722931 | 1722824 | − | conserved hypothetical protein |
| NMTN-01851 | SAV1619 | | 1725967 | 1728444 | 1726423 | − | hypothetical protein |
| NMTN-13184 | SAV1619 | | 1725967 | 1728444 | 1727261 | | |
| NMTN-09046 | SAV1619 | | 1725967 | 1728444 | 1727316 | | |
| NMTN-03924 | SAV1619 | | 1725967 | 1728444 | 1727472 | | |
| NMTN-12141 | SAV1620 | | 1728446 | 1729114 | 1728886 | − | conserved hypothetical protein |
| NMTN-12857t | SAV1620 | | 1728446 | 1729114 | 1728892 | | |
| NMTN-02539 | SAV1623 | | 1732376 | 1733401 | 1732569 | + | conserved hypothetical protein |
| NMTN-07404 | SAV1623 | | 1732376 | 1733401 | 1732832 | | |
| NMTN-00281 | SAV1623 | | 1732376 | 1733401 | 1733378 | | |
| NMTN-10429 | SAV1624 | | 1733633 | 1733779 | 1733623 | − | hypothetical protein |
| NMTN-10418 | SAV1624 | | 1733633 | 1733779 | 1733751 | | |
| NMTN-09949 | SAV1625 | csbD | 1733819 | 1734001 | 1733826 | − | sigmaB-controlled gene product |
| NMTN-09912 | SAV1627 | | 1734608 | 1735882 | 1734930 | + | conserved hypothetical protein |
| NMTN-03264 | SAV1627 | | 1734608 | 1735882 | 1734944 | | |
| NMTN-13834 | SAV1627 | | 1734608 | 1735882 | 1734986 | | |
| NMTN-03087 | SAV1627 | | 1734608 | 1735882 | 1735049 | | |
| NMTN-10053 | SAV1627 | | 1734608 | 1735882 | 1735413 | | |
| NMTN-09930 | SAV1627 | | 1734608 | 1735882 | 1735433 | | |
| NMTN-11865 | SAV1628 | | 1736043 | 1736816 | 1736441 | − | conserved hypothetical protein |
| NMTN-09724 | SAV1628 | | 1736043 | 1736816 | 1736664 | | |
| NMTN-02307 | SAV1632 | lytH | 1740783 | 1741658 | 1741078 | − | N-acetylmuramoyl-L-alanine amidase |
| NMTN-08882 | SAV1635 | apt | 1744736 | 1745254 | 1744894 | − | adenine phosphoribosyl transferase |
| NMTN-12680 | SAV1635 | apt | 1744736 | 1745254 | 1745237 | | |
| NMTN-14433 | SAV1636 | | 1745276 | 1747549 | 1745554 | − | hypothetical protein |
| NMTN-12480 | SAV1636 | | 1745276 | 1747549 | 1745716 | | |
| NMTN-00874 | SAV1636 | | 1745276 | 1747549 | 1746481 | | |
| NMTN-09928 | SAV1637 | secF | 1747752 | 1750031 | 1747796 | − | protein-export membrane protein SecDF |
| NMTN-09005 | SAV1638 | | 1750306 | 1750566 | 1750415 | − | conserved hypothetical protein |
| NMTN-02425 | SAV1638 | | 1750306 | 1750566 | 1750516 | | |
| NMTN-13469 | SAV1638 | | 1750306 | 1750566 | 1750541 | | |
| NMTN-03502 | SAV1638 | | 1750306 | 1750566 | 1750543 | | |
| NMTN-12732 | SAV1638 | | 1750306 | 1750566 | 1750543 | | |
| NMTN-10417 | SAV1639 | tgt | 1750585 | 1751724 | 1750652 | − | tRNA-guanine transglycosylase |
| NMTN-03576 | SAV1639 | tgt | 1750585 | 1751724 | 1750767 | | |
| NMTN-08610 | SAV1639 | tgt | 1750585 | 1751724 | 1750798 | | |
| NMTN-13096 | SAV1640 | queA | 1751747 | 1752772 | 1751749 | − | S-adenosylmethionine tRNA |
| | | | | | | | ribosyltransferase |
| NMTN-02724 | SAV1640 | queA | 1751747 | 1752772 | 1751851 | | |
| NMTN-11811 | SAV1640 | queA | 1751747 | 1752772 | 1752163 | | |
| NMTN-08942 | SAV1645 | rpmA | 1756540 | 1756839 | 1756710 | − | 50S ribosomal protei L27 |
| NMTN-04763 | SAV1651 | | 1760103 | 1760300 | 1760205 | + | hypothetical protein |
| NMTN-07932 | SAV1652 | | 1760417 | 1760701 | 1760414 | − | hypothetical protein |
| NMTN-10469 | SAV1652 | | 1760417 | 1760701 | 1760525 | | |
| NMTN-08113 | SAV1652 | | 1760417 | 1760701 | 1760703 | | |
| NMTN-09315 | SAV1652 | | 1760417 | 1760701 | 1760703 | | |
| NMTN-13114 | SAV1660 | truncated- | 1767593 | 1768147 | 1768048 | − | DNA repair protein RadC homolog |
| | | radC | | | | | |
| NMTN-13569 | SAV1661 | | 1768144 | 1768851 | 1768481 | − | hypothetical protein |
| NMTN-02079 | SAV1661 | | 1768144 | 1768851 | 1768572 | | |
| NMTN-05225 | SAV1661 | | 1768144 | 1768851 | 1768591 | | |
| NMTN-04873 | SAV1661 | | 1768144 | 1768851 | 1768806 | | |
| NMTN-11138 | SAV1664 | tag | 1773438 | 1773998 | 1773762 | + | DNA-3-methyladenine glycosidase |
| NMTN-09611 | SAV1664 | tag | 1773438 | 1773998 | 1773987 | | |
| NMTN-06627 | SAV1665 | | 1774221 | 1775294 | 1774327 | − | conserved hypothetical protein |
| NMTN-09830 | SAV1665 | | 1774221 | 1775294 | 1774404 | | |
| NMTN-02083 | SAV1665 | | 1774221 | 1775294 | 1774471 | | |
| NMTN-05323 | SAV1665 | | 1774221 | 1775294 | 1774504 | | |
| NMTN-05056 | SAV1665 | | 1774221 | 1775294 | 1774554 | | |
| NMTN-00482 | SAV1665 | | 1774221 | 1775294 | 1774557 | | |
| NMTN-10592 | SAV1665 | | 1774221 | 1775294 | 1774627 | | |
| NMTN-09296 | SAV1665 | | 1774221 | 1775294 | 1775009 | | |
| NMTN-00691 | SAV1667 | hemL | 1775490 | 1776776 | 1775814 | − | glutamate-1-semialdehyde 2,1- |
| | | | | | | | aminomutase |
| NMTN-06270 | SAV1674 | clpX | 1782603 | 1783865 | 1782623 | − | protease |
| NMTN-10448 | SAV1674 | clpX | 1782603 | 1783865 | 1782946 | | |
| NMTN-09787 | SAV1674 | clpX | 1782603 | 1783865 | 1783707 | | |
| NMTN-11361 | SAV1675 | tig | 1784016 | 1785317 | 1784101 | − | prolyl isomerase |
| NMTN-08815 | SAV1675 | tig | 1784016 | 1785317 | 1785131 | | |
| NMTN-06322 | SAV1675 | tig | 1784016 | 1785317 | 1785175 | | |
| NMTN-09198 | SAV1675 | tig | 1784016 | 1785317 | 1785294 | | |
| NMTN-06162 | SAV1676 | | 1785480 | 1786409 | 1785550 | − | hypothetical protein |
| NMTN-09142 | SAV1676 | | 1785480 | 1786409 | 1785688 | | |
| NMTN-12778 | SAV1676 | | 1785480 | 1786409 | 1785698 | | |
| NMTN-02739 | SAV1676 | | 1785480 | 1786409 | 1785759 | | |
| NMTN-04542 | SAV1676 | | 1785480 | 1786409 | 1785870 | | |
| NMTN-12560 | SAV1676 | | 1785480 | 1786409 | 1786358 | | |
| NMTN-00815 | SAV1676 | | 1785480 | 1786409 | 1786367 | | |
| NMTN-13130 | SAV1676 | | 1785480 | 1786409 | 1786379 | | |
| NMTN-13158 | SAV1676 | | 1785480 | 1786409 | 1786379 | | |
| NMTN-14282 | SAV1676 | | 1785480 | 1786409 | 1786408 | | |
| NMTN-07572 | SAV1676 | | 1785480 | 1786409 | 1786410 | | |
| NMTN-01639 | SAV1677 | | 1786428 | 1787036 | 1786655 | − | conserved hypothetical protein |
| NMTN-01982 | SAV1687 | gapB | 1795836 | 1796861 | 1796586 | − | glyceraldehyde 3-phosphate |
| | | | | | | | dehydrogenase 2 |
| NMTN-10389 | SAV1687 | gapB | 1795836 | 1796861 | 1796654 | | |
| NMTN-11111 | SAV1689 | | 1797670 | 1798542 | 1797866 | − | hypothetical protein |
| NMTN-13363 | SAV1689 | | 1797670 | 1798542 | 1798281 | | |
| NMTN-15238 | SAV1690 | polA | 1798558 | 1801188 | 1798599 | − | DNA polymerase I |
| NMTN-04682 | SAV1690 | polA | 1798558 | 1801188 | 1798871 | | |
| NMTN-08968 | SAV1690 | polA | 1798558 | 1801188 | 1799246 | | |
| NMTN-11891 | SAV1690 | polA | 1798558 | 1801188 | 1799441 | | |
| NMTN-11901 | SAV1690 | polA | 1798558 | 1801188 | 1799441 | | |
| NMTN-05636 | SAV1690 | polA | 1798558 | 1801188 | 1799848 | | |
| NMTN-02912 | SAV1690 | polA | 1798558 | 1801188 | 1800041 | | |
| NMTN-04778 | SAV1690 | polA | 1798558 | 1801188 | 1800365 | | |
| NMTN-10563 | SAV1690 | polA | 1798558 | 1801188 | 1800731 | | |
| NMTN-05527 | SAV1690 | polA | 1798558 | 1801188 | 1800756 | | |
| NMTN-07850 | SAV1691 | | 1801482 | 1802969 | 1801517 | − | hypothetical protein |
| NMTN-07842 | SAV1691 | | 1801482 | 1802969 | 1801520 | | |
| NMTN-11747 | SAV1691 | | 1801482 | 1802969 | 1801599 | | |
| NMTN-12759 | SAV1691 | | 1801482 | 1802969 | 1801774 | | |
| NMTN-07819 | SAV1691 | | 1801482 | 1802969 | 1801949 | | |
| NMTN-06260 | SAV1691 | | 1801482 | 1802969 | 1802347 | | |
| NMTN-11804 | SAV1692 | phoR | 1803468 | 1805132 | 1804598 | − | alkaline phosphatase synthesis sensor |
| | | | | | | | protein |
| NMTN-09369 | SAV1692 | phoR | 1803468 | 1805132 | 1804907 | | |
| NMTN-03695 | SAV1692 | phoR | 1803468 | 1805132 | 1805037 | | |
| NMTN-14551 | SAV1693 | phoP | 1805129 | 1805833 | 1805388 | − | alkaline phosphatase synthesis |
| | | | | | | | transcriptional regulatory protein |
| NMTN-11620 | SAV1694 | citC | 1806223 | 1807491 | 1807042 | − | isocitrate dehyrogenase |
| NMTN-09707 | SAV1694 | citC | 1806223 | 1807491 | 1807346 | | |
| NMTN-05271 | SAV1694 | citC | 1806223 | 1807491 | 1807368 | | |
| NMTN-06106 | SAV1695 | citZ | 1807540 | 1808661 | 1807907 | | |
| NMTN-14679 | SAV1695 | citZ | 1807540 | 1808661 | 1807932 | | |
| NMTN-06897 | SAV1695 | citZ | 1807540 | 1808661 | 1808045 | | |
| NMTN-06431 | SAV1695 | citZ | 1807540 | 1808661 | 1808413 | | |
| NMTN-08586 | SAV1695 | citZ | 1807540 | 1808661 | 1808426 | | |
| NMTN-11247 | SAV1695 | citZ | 1807540 | 1808661 | 1808454 | | |
| NMTN-13092 | SAV1697 | pykA | 1810718 | 1812475 | 1810778 | − | pyruvate kinase |
| NMTN-15191 | SAV1697 | pykA | 1810718 | 1812475 | 1812204 | | |
| NMTN-01819 | SAV1706 | | 1824903 | 1825316 | 1825069 | + | conserved hypothetical protein |
| NMTN-14013 | SAV1707 | | 1825621 | 1826310 | 1826247 | − | conserved hypothetical protein |
| NMTN-10478 | SAV1708 | | 1826464 | 1827540 | 1827380 | + | Xaa-Pro dipeptidase homolog |
| NMTN-10382 | SAV1709 | ald | 1827857 | 1828975 | 1828429 | − | alanine dehydrogenase |
| NMTN-07146 | SAV1709 | ald | 1827857 | 1828975 | 1828464 | | |
| NMTN-02179 | SAV1709 | ald | 1827857 | 1828975 | 1828697 | | |
| NMTN-10024 | SAV1710 | | 1829116 | 1829616 | 1829317 | + | conserved hypothetical protein |
| NMTN-14643 | SAV1712 | | 1831152 | 1832099 | 1831520 | − | conserved hypothetical protein |
| NMTN-13852 | SAV1712 | | 1831152 | 1832099 | 1831979 | | |
| NMTN-09686 | SAV1712 | | 1831152 | 1832099 | 1832067 | | |
| NMTN-09756 | SAV1713 | | 1832222 | 1832716 | 1832322 | − | hypothetical protein |
| NMTN-12153 | SAV1714 | | 1832814 | 1833584 | 1832950 | − | conserved hypothetical protein |
| NMTN-00831 | SAV1716 | | 1834851 | 1835990 | 1835980 | − | hypothetical protein |
| NMTN-13546 | SAV1718 | | 1838194 | 1838658 | 1838305 | + | conserved hypothetical protein |
| NMTN-09295 | SAV1721 | | 1841240 | 1841983 | 1841491 | − | hypothetical protein |
| NMTN-06110 | SAV1722 | | 1842063 | 1842509 | 1842141 | + | conserved hypothetical protein |
| NMTN-12565 | SAV1723 | | 1842624 | 1843781 | 1843309 | + | hypothetical protein |
| NMTN-09575 | SAV1723 | | 1842624 | 1843781 | 1843660 | | |
| NMTN-02692 | SAV1723 | | 1842624 | 1843781 | 1843730 | | |
| NMTN-08670 | SAV1724 | serA | 1843768 | 1845372 | 1844323 | + | D-3-phosphoglycerate dehydrogenase |
| NMTN-05575 | SAV1724 | serA | 1843768 | 1845372 | 1844864 | | |
| NMTN-10466 | SAV1724 | serA | 1843768 | 1845372 | 1844881 | | |
| NMTN-11691 | SAV1724 | serA | 1843768 | 1845372 | 1845299 | | |
| NMTN-09771 | SAV1725 | | 1845522 | 1846652 | 1845768 | − | hypothetical protein |
| NMTN-10054 | SAV1725 | | 1845522 | 1846652 | 1845772 | | |
| NMTN-14446 | SAV1725 | | 1845522 | 1846652 | 1846574 | | |
| NMTN-01769 | SAV1726 | ptaA | 1846759 | 1848225 | 1846835 | − | PTS system, N-acetylglucosamine- |
| | | | | | | | specific IIABC component |
| NMTN-10689 | SAV1726 | ptaA | 1846759 | 1848225 | 1847009 | | |
| NMTN-06593 | SAV1726 | ptaA | 1846759 | 1848225 | 1847429 | | |
| NMTN-12848 | SAV1726 | ptaA | 1846759 | 1848225 | 1847724 | | |
| NMTN-12216 | SAV1726 | ptaA | 1846759 | 1848225 | 1848081 | | |
| NMTN-12497 | SAV1726 | ptaA | 1846759 | 1848225 | 1848081 | | |
| NMTN-10697 | SAV1728 | | 1849142 | 1850416 | 1849284 | + | hypothetical protein |
| NMTN-11661 | SAV1728 | | 1849142 | 1850416 | 1849772 | | |
| NMTN-05535 | SAV1728 | | 1849142 | 1850416 | 1849919 | | |
| NMTN-02036 | SAV1728 | | 1849142 | 1850416 | 1850072 | | |
| NMTN-09264 | SAV1730 | sgtA | 1852154 | 1853059 | 1852198 | + | probable transglycosylase |
| NMTN-14018 | SAV1730 | sgtA | 1852154 | 1853059 | 1852334 | | |
| NMTN-05285 | SAV1730 | sgtA | 1852154 | 1853059 | 1852650 | | |
| NMTN-09211 | SAV1730 | sgtA | 1852154 | 1853059 | 1852725 | | |
| NMTN-09729 | SAV1730 | sgtA | 1852154 | 1853059 | 1852799 | | |
| | or | | | | | | |
| | SAV0504 | | | | | | |
| NMTN-13044 | SAV1730 | sgtA | 1852154 | 1853059 | 1853025 | | |
| NMTN-04121 | SAV1731 | sasl | 1853262 | 1855937 | 1853455 | − | hypothetical protein |
| NMTN-04340 | SAV1731 | sasl | 1853262 | 1855937 | 1853675 | | |
| NMTN-06315 | SAV1731 | sasl | 1853262 | 1855937 | 1854698 | | |
| NMTN-05519 | SAV1731 | sasl | 1853262 | 1855937 | 1854778 | | |
| NMTN-00742 | SAV1731 | sasl | 1853262 | 1855937 | 1855414 | | |
| NMTN-13827 | SAV1731 | sasl | 1853262 | 1855937 | 1855619 | | |
| NMTN-01649 | SAV1732 | fhs | 1856270 | 1857937 | 1856841 | − | formyltetrahydrofolate synthetase |
| NMTN-04341 | SAV1732 | fhs | 1856270 | 1857937 | 1857495 | | |
| NMTN-11263 | SAV1733 | acsA | 1858377 | 1860083 | 1858767 | − | acetyl-CoA synthetase |
| NMTN-10957 | SAV1733 | acsA | 1858377 | 1860083 | 1858898 | | |
| NMTN-01614 | SAV1733 | acsA | 1858377 | 1860083 | 1859552 | | |
| NMTN-04398 | SAV1733 | acsA | 1858377 | 1860083 | 1859626 | | |
| NMTN-03779 | SAV1733 | acsA | 1858377 | 1860083 | 1859771 | | |
| NMTN-02713 | SAV1733 | acsA | 1858377 | 1860083 | 1859863 | | |
| NMTN-01564 | SAV1733 | acsA | 1858377 | 1860083 | 1859993 | | |
| NMTN-00456 | SAV1733 | acsA | 1858377 | 1860083 | 1860019 | | |
| NMTN-09376 | SAV1734 | acuA | 1860252 | 1860884 | 1860638 | + | acetoin dehydrogenase homolog |
| NMTN-09382 | SAV1734 | acuA | 1860252 | 1860884 | 1860638 | | |
| NMTN-09642 | SAV1734 | acuA | 1860252 | 1860884 | 1860742 | | |
| NMTN-03717 | SAV1735 | acuC | 1860909 | 1862078 | 1861575 | + | acetoin utilization protein |
| NMTN-11458 | SAV1735 | acuC | 1860909 | 1862078 | 1861987 | | |
| NMTN-06725 | SAV1736 | ccpA | 1862176 | 1863120 | 1862328 | − | catabolite control protein A |
| NMTN-11822 | SAV1736 | ccpA | 1862176 | 1863120 | 1862951 | | |
| NMTN-07791 | SAV1736 | ccpA | 1862176 | 1863120 | 1863051 | | |
| NMTN-12369 | SAV1744 | | 1874251 | 1874562 | 1874338 | − | thioredoxin homolog |
| NMTN-11861 | SAV1745 | | 1874627 | 1875703 | 1874711 | − | endo-1,4-beta-glucanase homolog |
| NMTN-09978 | SAV1745 | | 1874627 | 1875703 | 1875150 | | |
| NMTN-09049 | SAV1745 | | 1874627 | 1875703 | 1875179 | | |
| NMTN-13099 | SAV1746 | | 1875790 | 1876101 | 1876022 | + | hypothetical protein |
| NMTN-02504 | SAV1747 | | 1876226 | 1877068 | 1876403 | − | conserved hypothetical protein |
| NMTN-05495 | SAV1747 | | 1876226 | 1877068 | 1876527 | | |
| NMTN-13578 | SAV1747 | | 1876226 | 1877068 | 1876885 | | |
| NMTN-08693 | SAV1750 | | 1879539 | 1880387 | 1879560 | − | D-alanine aminotransferase |
| NMTN-02855 | SAV1750 | | 1879539 | 1880387 | 1879583 | | |
| NMTN-04451 | SAV1750 | | 1879539 | 1880387 | 1879783 | | |
| NMTN-15243 | SAV1750 | | 1879539 | 1880387 | 1879789 | | |
| NMTN-11784 | SAV1750 | | 1879539 | 1880387 | 1879807 | | |
| NMTN-00182 | SAV1750 | | 1879539 | 1880387 | 1880096 | | |
| NMTN-00502 | SAV1750 | | 1879539 | 1880387 | 1880156 | | |
| NMTN-00370 | SAV1750 | | 1879539 | 1880387 | 1880272 | | |
| NMTN-06678 | SAV1751 | | 1880391 | 1881800 | 1880460 | − | Xaa-His dipeptidase homolog |
| NMTN-06780 | SAV1752 | | 1882358 | 1882780 | 1882743 | − | hypothetical protein |
| NMTN-13733 | SAV1753 | | 1882797 | 1883492 | 1882995 | − | hypothetical protein |
| NMTN-03697 | SAV1753 | | 1882797 | 1883492 | 1882998 | | |
| NMTN-03027 | SAV1753 | | 1882797 | 1883492 | 1883348 | | |
| NMTN-01581 | SAV1755 | | 1885515 | 1886459 | 1885702 | + | hypothetical protein |
| NMTN-04050 | SAV1755 | | 1885515 | 1886459 | 1885707 | | |
| NMTN-07832 | SAV1755 | | 1885515 | 1886459 | 1886032 | | |
| NMTN-06625 | SAV1755 | | 1885515 | 1886459 | 1886427 | | |
| NMTN-01809 | SAV1756 | | 1886434 | 1886706 | 1886450 | + | conserved hypothetical protein |
| NMTN-11099 | SAV1758 | mrp | 1886823 | 1887464 | 1886845 | − | Mrp protein |
| NMTN-09839 | SAV1758 | mrp | 1886823 | 1887464 | 1886975 | | |
| NMTN-11819 | SAV1758 | mrp | 1886823 | 1887464 | 1887207 | | |
| NMTN-08746 | SAV1758 | mrp | 1886823 | 1887464 | 1887262 | | |
| NMTN-04980 | SAV1758 | mrp | 1886823 | 1887464 | 1887275 | | |
| NMTN-12007 | SAV1758 | mrp | 1887431 | 1893382 | 1887895 | | |
| NMTN-14526 | SAV1758 | mrp | 1887431 | 1893382 | 1889271 | | |
| NMTN-07809 | SAV1758 | mrp | 1887431 | 1893382 | 1889355 | | |
| NMTN-07511 | SAV1758 | mrp | 1887431 | 1893382 | 1889664 | | |
| NMTN-11707 | SAV1758 | mrp | 1886823 | 1893382 | 1889664 | | |
| NMTN-00816 | SAV1758 | mrp | 1887431 | 1893382 | 1889902 | | |
| NMTN-00208 | SAV1758 | mrp | 1887431 | 1893382 | 1891275 | | |
| NMTN-00430 | SAV1758 | mrp | 1887431 | 1893382 | 1891275 | | |
| NMTN-00485 | SAV1758 | mrp | 1887431 | 1893382 | 1891275 | | |
| NMTN-05106 | SAV1758 | mrp | 1887431 | 1893382 | 1891635 | | |
| NMTN-15347 | SAV1758 | mrp | 1887431 | 1893382 | 1891947 | | |
| NMTN-06964 | SAV1759 | | 1893708 | 1894019 | 1893709 | − | conserved hypothetical protein |
| NMTN-14685 | SAV1761 | | 1896746 | 1897927 | 1897039 | − | multidrug resistance protein homolog |
| NMTN-09423 | SAV1761 | | 1896746 | 1897927 | 1897202 | | |
| NMTN-03453 | SAV1761 | | 1896746 | 1897927 | 1897203 | | |
| NMTN-12063d | SAV1761 | | 1896746 | 1897927 | 1897424 | | |
| NMTN-04981 | SAV1761 | | 1896746 | 1897927 | 1897669 | | |
| NMTN-09444 | SAV1762 | | 1898037 | 1898990 | 1898865 | + | conserved hypothetical protein |
| NMTN-00074 | SAV1762 | | 1898037 | 1898990 | 1898913 | | |
| NMTN-11223 | SAV1762 | | 1898987 | 1899550 | 1899036 | + | conserved hypothetical protein |
| NMTN-05156 | SAV1763 | | 1898987 | 1899550 | 1899036 | | |
| NMTN-11223 | SAV1763 | | 1898987 | 1899550 | 1899036 | | |
| NMTN-04006 | SAV1763 | | 1898987 | 1899550 | 1899162 | | |
| NMTN-08392 | SAV1763 | | 1898987 | 1899550 | 1899249 | | |
| NMTN-06245 | SAV1763 | | 1898987 | 1899550 | 1899359 | | |
| NMTN-04041 | SAV1763 | | 1898987 | 1899550 | 1899384 | | |
| NMTN-05428 | SAV1763 | | 1898987 | 1899550 | 1899395 | | |
| NMTN-11740 | SAV1765 | | 1900644 | 1901471 | 1901217 | − | lysophospholipase homolog |
| NMTN-07701 | SAV1765 | | 1900644 | 1901471 | 1901391 | | |
| NMTN-13818 | SAV1766 | | 1901705 | 1902706 | 1901721 | + | proline dehydrohenase homolog |
| NMTN-12386 | SAV1766 | | 1901705 | 1902706 | 1902093 | | |
| NMTN-06814 | SAV1766 | | 1901705 | 1902706 | 1902230 | | |
| NMTN-00862 | SAV1768 | | 1903201 | 1903293 | 1903245 | − | 6,7-dimethyl-8-ribityllumazine synthase |
| NMTN-06176 | SAV1769 | ribA | 1903306 | 1904487 | 1903322 | − | riboflavin biosynthesis protein |
| NMTN-06184 | SAV1769 | ribA | 1903306 | 1904487 | 1903322 | | |
| NMTN-11586 | SAV1769 | ribA | 1903306 | 1904487 | 1903322 | | |
| NMTN-14759 | SAV1769 | ribA | 1903306 | 1904487 | 1904319 | | |
| NMTN-04855 | SAV1770 | ribB | 1904498 | 1905130 | 1904524 | − | riboflavin synthase alpha chain |
| NMTN-06309 | SAV1770 | ribB | 1904498 | 1905130 | 1904692 | | |
| NMTN-11573 | SAV1770 | ribB | 1904498 | 1905130 | 1904730 | | |
| NMTN-11553 | SAV1771 | ribD | 1905137 | 1906180 | 1905569 | − | riboflavin specific deaminase |
| NMTN-03534 | SAV1771 | ribD | 1905137 | 1906180 | 1905582 | | |
| NMTN-15236 | SAV1771 | ribD | 1905137 | 1906180 | 1905883 | | |
| NMTN-10921 | SAV1772 | | 1906661 | 1908163 | 1906806 | − | hypothetical protein |
| NMTN-10652 | SAV1772 | | 1906661 | 1908163 | 1906987 | | |
| NMTN-02541 | SAV1772 | | 1906661 | 1908163 | 1906990 | | |
| NMTN-01922 | SAV1772 | | 1906661 | 1908163 | 1906996 | | |
| NMTN-02899 | SAV1772 | | 1906661 | 1908163 | 1907621 | | |
| NMTN-11737 | SAV1772 | | 1906661 | 1908163 | 1907667 | | |
| NMTN-06673 | SAV1772 | | 1906661 | 1908163 | 1907691 | | |
| NMTN-11489 | SAV1772 | | 1906661 | 1908163 | 1907752 | | |
| NMTN-06844 | SAV1772 | | 1906661 | 1908163 | 1908162 | | |
| NMTN-08006 | SAV1773 | | 1908686 | 1909000 | 1908852 | + | arsenical resistance operon repressor |
| | | | | | | | homolog |
| NMTN-11851 | SAV1773 | | 1908686 | 1909000 | 1908942 | | |
| NMTN-03743 | SAV1774 | | 1909000 | 1910292 | 1909296 | + | aesenical pump membrane protein |
| | | | | | | | homolog |
| NMTN-05075 | SAV1774 | | 1909000 | 1910292 | 1909369 | | |
| NMTN-12218 | SAV1774 | | 1909000 | 1910292 | 1909553 | | |
| NMTN-12716 | SAV1774 | | 1909000 | 1910292 | 1909817 | | |
| NMTN-08965 | SAV1774 | | 1909000 | 1910292 | 1909872 | | |
| NMTN-05013 | SAV1774 | | 1909000 | 1910292 | 1909875 | | |
| NMTN-13382 | SAV1774 | | 1909000 | 1910292 | 1909875 | | |
| NMTN-04924 | SAV1784 | | 1916652 | 1917005 | 1916805 | + | hypothetical protein |
| NMTN-02929 | SAV1785 | | 1917467 | 1917817 | 1917728 | + | truncated transposase |
| NMTN-12695 | SAV1787 | | 1918160 | 1918939 | 1918170 | + | truncated transposase |
| NMTN-11090 | SAV1787 | | 1918160 | 1918939 | 1918311 | | |
| NMTN-08318 | SAV1787 | | 1918160 | 1918939 | 1918344 | | |
| NMTN-05157 | SAV1787 | | 1918160 | 1918939 | 1918467 | | |
| NMTN-09018 | SAV1787 | | 1918160 | 1918939 | 1918476 | | |
| NMTN-11190 | SAV1787 | | 1918160 | 1918939 | 1918477 | | |
| NMTN-05559 | SAV1787 | | 1918160 | 1918939 | 1918521 | | |
| NMTN-13297 | SAV1787 | | 1918160 | 1918939 | 1918665 | | |
| NMTN-10381 | SAV1787 | | 1918160 | 1918939 | 1918754 | | |
| NMTN-13264 | SAV1788 | | 1919002 | 1919835 | 1919428 | − | plant metabolite dehydrogenase |
| | | | | | | | homolog |
| NMTN-01969 | SAV1791 | pckA | 1922645 | 1924237 | 1923229 | + | phosphoenolpyruvate carboxykinase |
| NMTN-08719 | SAV1791 | pckA | 1922645 | 1924237 | 1923548 | | |
| NMTN-09685 | SAV1791 | pckA | 1922645 | 1924237 | 1924070 | | |
| NMTN-11646 | SAV1793 | | 1924615 | 1925385 | 1925085 | − | conserved hypothetical protein |
| NMTN-05533 | SAV1793 | | 1924615 | 1925385 | 1925223 | | |
| NMTN-07557 | SAV1794 | | 1925366 | 1925845 | 1925588 | − | conserved hypothetical protein |
| NMTN-13076 | SAV1794 | | 1925366 | 1925845 | 1925793 | | |
| NMTN-13249 | SAV1796 | menC | 1926159 | 1927160 | 1926179 | − | o-succinylbenzoic acid synthetase |
| NMTN-11082 | SAV1798 | | 1928802 | 1929284 | 1929118 | − | hypothetical protein |
| NMTN-04503 | SAV1801 | | 1931361 | 1931987 | 1931506 | + | hypothetical protein |
| NMTN-04945 | SAV1801 | | 1931362 | 1931988 | 1931785 | | |
| NMTN-04108 | SAV1803 | | 1932470 | 1933042 | 1932649 | + | hypothetical protein |
| NMTN-11925 | SAV1803 | | 1932470 | 1933042 | 1932778 | | |
| NMTN-04822 | SAV1803 | | 1932470 | 1933042 | 1932816 | | |
| NMTN-05478 | SAV1805 | | 1933831 | 1934085 | 1933953 | − | truncated transposase |
| NMTN-12446 | SAV1805 | | 1933831 | 1934085 | 1934009 | | |
| NMTN-13371 | SAV1805 | | 1933831 | 1934085 | 1934022 | | |
| NMTN-04948 | SAV1807 | | 1935792 | 1937021 | 1936510 | − | probable specificity determinant HsdS |
| NMTN-09595 | SAV1807 | | 1935792 | 1937021 | 1936682 | | |
| NMTN-06428 | SAV1807 | | 1935792 | 1937021 | 1936732 | | |
| NMTN-03260 | SAV1808 | | 1937014 | 1938570 | 1937248 | − | type I restriction enzyme EcoR124II M |
| | | | | | | | protein homolog |
| NMTN-11963 | SAV1808 | | 1937014 | 1938570 | 1937663 | | |
| NMTN-05473 | SAV1808 | | 1937014 | 1938570 | 1937798 | | |
| NMTN-12382 | SAV1808 | | 1937014 | 1938570 | 1938019 | | |
| | or | | | | | | |
| | SAV0431 | | | | | | |
| NMTN-06104 | SAV1809 | splF | 1938931 | 1939650 | 1939158 | − | serine protease |
| NMTN-04782 | SAV1809/ | splF/splD | 1939808 | 1940527 | 1939202 | − | serine protease |
| | 1810 | | | | | | |
| NMTN-02814 | SAV1809 | | | | 1940294 | | |
| | or | | | | | | |
| | SAV1810 | | | | | | |
| NMTN-03294 | SAV1811 | splC | 1940648 | 1941367 | 1940807 | − | serine protease |
| NMTN-11075 | SAV1811 | splC | 1940648 | 1941367 | 1941075 | | |
| NMTN-12789 | SAV1813 | splA | 1942272 | 1942979 | 1942431 | − | serine protease |
| NMTN-01785 | SAV1814 | | 1943415 | 1943633 | 1943495 | − | hypothetical protein |
| NMTN-03169 | SAV1815 | | 1943942 | 1944523 | 1944173 | + | probable beta-lactamase |
| NMTN-14630d | SAV1819 | lukD | 1947093 | 1948091 | 1947646 | − | leukotoxin F-subunit |
| NMTN-13601 | SAV1819 | lukD | 1947093 | 1948091 | 1947863 | | |
| NMTN-14661 | SAV1820 | lukE | 1948093 | 1949028 | 1948233 | − | leukotoxin S-subunit |
| NMTN-08984 | SAV1820 | lukE | 1948093 | 1949028 | 1948416 | | |
| NMTN-03521 | SAV1821 | | 1950392 | 1951180 | 1950522 | + | hypothetical protein |
| NMTN-09153 | SAV1835 | traP | 1965438 | 1965941 | 1965756 | + | signal transduction protein |
| NMTN-07467 | SAV1835 | traP | 1965438 | 1965941 | 1965844 | | |
| NMTN-15242 | SAV1838 | hit | 1968155 | 1968577 | 1968248 | + | Hit-like protein involved in cell-cycle |
| | | | | | | | regulation |
| NMTN-01973 | SAV1838 | hit | 1968155 | 1968577 | 1968258 | | |
| NMTN-11581 | SAV1839 | | 1968719 | 1969084 | 1968910 | + | conserved hypothetical protein |
| NMTN-10849 | SAV1840 | | 1969819 | 1970376 | 1969919 | + | conserved hypothetical protein |
| NMTN-09667 | SAV1840 | | 1969819 | 1970376 | 1970043 | | |
| NMTN-10393 | SAV1841 | prsA | 1970581 | 1971543 | 1971420 | + | peptidyl-prolyl cis/trans isomerase |
| NMTN-09920 | SAV1842 | | 1971664 | 1972605 | 1971667 | − | cmp-binding-factor 1 |
| NMTN-07865 | SAV1843 | | 1972602 | 1975538 | 1972862 | − | conserved hypothetical protein |
| NMTN-13866 | SAV1843 | | 1972602 | 1975538 | 1972923 | | |
| NMTN-07734 | SAV1843 | | 1972602 | 1975538 | 1973119 | | |
| NMTN-07512 | SAV1843 | | 1972602 | 1975538 | 1973162 | | |
| NMTN-12781d | SAV1843 | | 1972602 | 1975538 | 1973162 | | |
| NMTN-10644 | SAV1843 | | 1972602 | 1975538 | 1974985 | | |
| NMTN-02336 | SAV1843 | | 1972602 | 1975538 | 1975094 | | |
| NMTN-02128 | SAV1843 | | 1972602 | 1975538 | 1975298 | | |
| NMTN-00631 | SAV1843 | | 1972602 | 1975538 | 1975322 | | |
| NMTN-01518 | SAV1844 | | 1975528 | 1976724 | 1975644 | − | conserved hypothetical protein |
| NMTN-02947 | SAV1844 | | 1975528 | 1976724 | 1975719 | | |
| NMTN-07909 | SAV1844 | | 1975528 | 1976724 | 1975971 | | |
| NMTN-08718 | SAV1844 | | 1975528 | 1976724 | 1976096 | | |
| NMTN-06479 | SAV1844 | | 1975528 | 1976724 | 1976133 | | |
| NMTN-13817 | SAV1844 | | 1975528 | 1976724 | 1976464 | | |
| NMTN-03798 | SAV1844 | | 1975528 | 1976724 | 1976526 | | |
| NMTN-08153 | SAV1846 | | 1978065 | 1979189 | 1978174 | − | conserved hypothetical protein |
| NMTN-04904 | SAV1846 | | 1978065 | 1979189 | 1979077 | | |
| NMTN-09366 | SAV1848 | | 1980189 | 1980812 | 1980192 | − | two-component response regulator |
| | | | | | | | homolog |
| NMTN-06189 | SAV1848 | | 1980189 | 1980812 | 1980606 | | |
| NMTN-06325 | SAV1849 | | 1980834 | 1981946 | 1981010 | − | two-component sensor histidine kinase |
| | | | | | | | homolog |
| NMTN-02615 | SAV1849 | | 1980834 | 1981946 | 1981012 | | |
| NMTN-03790 | SAV1849 | | 1980834 | 1981946 | 1981015 | | |
| NMTN-13819 | SAV1849 | | 1980834 | 1981946 | 1981538 | | |
| NMTN-13245 | SAV1850 | | 1982109 | 1982930 | 1982306 | + | conserved hypothetical protein |
| NMTN-01596 | SAV1851 | citG | 1983383 | 1984768 | 1983541 | − | fumarate hydratase, class-II |
| NMTN-03937 | SAV1851 | citG | 1983383 | 1984768 | 1984552 | | |
| NMTN-09007 | SAV1851 | citG | 1983383 | 1984768 | 1984621 | | |
| NMTN-13064 | SAV1852 | | 1984964 | 1985359 | 1985016 | − | hypothetical protein |
| NMTN-03642 | SAV18S2 | | 1984964 | 1985359 | 1985017 | | |
| NMTN-03650 | SAV1852 | | 1984964 | 1985359 | 1985017 | | |
| NMTN-04347 | SAV1852 | | 1984964 | 1985359 | 1985021 | | |
| NMTN-13036 | SAV1852 | | 1984964 | 1985359 | 1985056 | | |
| NMTN-09688 | SAV1852 | | 1984964 | 1985359 | 1985111 | | |
| NMTN-07774 | SAV1852 | | 1984964 | 1985359 | 1985119 | | |
| NMTN-07515 | SAV1852 | | 1984964 | 1985359 | 1985253 | | |
| NMTN-12503 | SAV18S2 | | 1984964 | 1985359 | 1985264 | | |
| NMTN-12935 | SAV1852 | | 1984964 | 1985359 | 1985326 | | |
| NMTN-00748 | SAV1852 | | 1984964 | 1985359 | 1985335 | | |
| NMTN-10202 | SAV1852 | | 1984964 | 1985359 | 1985354 | | |
| NMTN-11183 | SAV1854 | | 1986084 | 1986683 | 1986247 | − | hypothetical protein |
| NMTN-04023 | SAV1856 | | 1987317 | 1988444 | 1987741 | − | conserved hypothetical protein |
| NMTN-09126 | SAV1856 | | 1987317 | 1988444 | 1987925 | | |
| NMTN-09706 | SAV1857 | | 1988595 | 1989323 | 1989075 | − | glutamate ABC transporter ATP-binding |
| | | | | | | | protein |
| NMTN-15183 | SAV1858 | | 1989310 | 1990635 | 1989944 | − | hypothetical protein |
| NMTN-09524 | SAV1858 | | 1989310 | 1990635 | 1990417 | | |
| NMTN-12582 | SAV1859 | | 1991025 | 1992086 | 1991028 | − | hypothetical protein |
| NMTN-12615 | SAV1859 | | 1991025 | 1992086 | 1991665 | | |
| NMTN-05062 | SAV1859 | | 1991025 | 1992086 | 1991796 | | |
| NMTN-02488 | SAV1859 | | 1991025 | 1992086 | 1991893 | | |
| NMTN-06458 | SAV1699/ | tnp | 1992670 | 1993989 | 1992885 | + | transposase |
| | 1720/ | | | | | | |
| | 1860 | | | | | | |
| NMTN-09121 | | | 1994457 | 1994528 | 1994472 | − | tRNA-glycine |
| NMTN-08450 | SAV1862 | | 2003031 | 2003981 | 2003064 | − | hypothetical protein |
| NMTN-04175 | SAV1862 | | 2003031 | 2003981 | 2003312 | | |
| NMTN-12948 | SAV1862 | | 2003031 | 2003981 | 2003608 | | |
| NMTN-01520 | SAV1862 | | 2003031 | 2003981 | 2003854 | | |
| NMTN-06757 | SAV1863 | | 2003987 | 2004442 | 2004312 | − | conserved hypothetical protein |
| NMTN-05104 | SAV1863 | | 2003987 | 2004442 | 2004441 | | |
| NMTN-15185d | SAV1864 | gsaB | 2004523 | 2005812 | 2004693 | + | glutamate-1-semialdehyde |
| | | | | | | | aminotransferase |
| NMTN-01784 | SAV1865 | | 2006105 | 2007199 | 2006154 | + | conserved hypothetical protein |
| NMTN-14615 | SAV1865 | | 2006105 | 2007199 | 2006235 | | |
| NMTN-06367 | SAV1865 | | 2006105 | 2007199 | 2006288 | | |
| NMTN-06370 | SAV1865 | | 2006105 | 2007199 | 2006288 | | |
| NMTN-05465 | SAV1865 | | 2006105 | 2007199 | 2006303 | | |
| NMTN-08780 | SAV1865 | | 2006105 | 2007199 | 2006376 | | |
| NMTN-13338 | SAV1865 | | 2006105 | 2007199 | 2006423 | | |
| NMTN-09201 | SAV1865 | | 2006105 | 2007199 | 2006641 | | |
| NMTN-09272 | SAV1865 | | 2006105 | 2007199 | 2006643 | | |
| NMTN-14420 | SAV1865 | | 2006105 | 2007199 | 2006666 | | |
| NMTN-02750 | SAV1865 | | 2006105 | 2007199 | 2006891 | | |
| NMTN-06155 | SAV1865 | | 2006105 | 2007199 | 2007155 | | |
| NMTN-11093 | SAV1865 | | 2006105 | 2007199 | 2007190 | | |
| NMTN-01574 | SAV1866 | | 2007389 | 2009125 | 2007529 | − | ABC transporter homolog |
| NMTN-09304 | SAV1866 | | 2007389 | 2009125 | 2008258 | | |
| NMTN-08811 | SAV1866 | | 2007389 | 2009125 | 2008620 | | |
| NMTN-12175 | SAV1866 | | 2007389 | 2009125 | 2008885 | | |
| NMTN-04859 | SAV1866 | | 2007389 | 2009125 | 2009009 | | |
| NMTN-06107 | SAV1868 | | 2010261 | 2011298 | 2010295 | − | hypothetical protein |
| NMTN-00194 | SAV1868 | | 2010261 | 2011298 | 2010307 | | |
| NMTN-11037 | SAV1868 | | 2010261 | 2011298 | 2010988 | | |
| NMTN-10276 | SAV1868 | | 2010261 | 2011298 | 2011050 | | |
| NMTN-07273d | SAV1870 | | 2012688 | 2013524 | 2013200 | − | hypothetical protein |
| NMTN-03703 | SAV1870 | | 2012688 | 2013524 | 2013403 | | |
| NMTN-12992 | SAV1871 | | 2013533 | 2015050 | 2013798 | − | hypothetical protein |
| NMTN-12899 | SAV1873 | | 2015357 | 2016175 | 2015480 | − | conserved hypothetical protein |
| NMTN-06284 | SAV1874 | | 2016435 | 2017244 | 2016975 | − | conserved hypothetical protein |
| NMTN-08476 | SAV1874 | | 2016435 | 2017244 | 2017224 | | |
| NMTN-03774 | SAV1875 | | 2017584 | 2018099 | 2017763 | − | conserved hypothetical protein |
| NMTN-12914 | SAV1875 | | 2017584 | 2018099 | 2017918 | | |
| NMTN-02538 | SAV1877 | | 2018615 | 2019766 | 2018949 | + | conserved hypothetical protein |
| NMTN-06267 | SAV1877 | | 2018615 | 2019766 | 2019026 | | |
| NMTN-10893 | SAV1877 | | 2018615 | 2019766 | 2019224 | | |
| NMTN-09071 | SAV1877 | | 2018615 | 2019766 | 2019273 | | |
| NMTN-06180 | SAV1877 | | 2018615 | 2019766 | 2019383 | | |
| NMTN-06376 | SAV1877 | | 2018615 | 2019766 | 2019558 | | |
| NMTN-12444 | SAV1877 | | 2018615 | 2019766 | 2019735 | | |
| NMTN-12278 | SAV1879 | ampS | 2020646 | 2021893 | 2020927 | − | aminopeptidase ampS |
| NMTN-11495d | SAV1879 | ampS | 2020646 | 2021893 | 2021055 | | |
| NMTN-05290 | SAV1879 | ampS | 2020646 | 2021893 | 2021091 | | |
| NMTN-09410 | SAV1879 | ampS | 2020646 | 2021893 | 2021649 | | |
| NMTN-03400 | SAV1879 | ampS | 2020646 | 2021893 | 2021722 | | |
| NMTN-03809 | SAV1880 | | 2021905 | 2022111 | 2021991 | − | conserved hypothetical protein |
| NMTN-12085 | SAV1880 | | 2021905 | 2022111 | 2021993 | | |
| NMTN-12484 | SAV1880 | | 2021905 | 2022111 | 2021993 | | |
| NMTN-12080 | SAV1880 | | 2021905 | 2022111 | 2021995 | | |
| NMTN-03565 | SAV1880 | | 2021905 | 2022111 | 2021996 | | |
| NMTN-03575 | SAV1880 | | 2021905 | 2022111 | 2021996 | | |
| NMTN-15208 | SAV1881 | | 2022252 | 2022716 | 2022288 | + | hypothetical protein |
| NMTN-06353 | SAV1883 | | 2023276 | 2024493 | 2023391 | + | hypothetical protein |
| NMTN-13242 | SAV1883 | | 2023276 | 2024493 | 2023391 | | |
| NMTN-10508 | SAV1883 | | 2023276 | 2024493 | 2024475 | | |
| NMTN-02993 | SAV1884 | vraR | 2024606 | 2025235 | 2024988 | − | two-component response regulator |
| NMTN-07438 | SAV1885 | vraS | 2025225 | 2026268 | 2025278 | − | two-component sensor histidine kinase |
| NMTN-02364 | SAV1885 | vraS | 2025225 | 2026268 | 2025901 | | |
| NMTN-11639 | SAV1885 | vraS | 2025225 | 2026268 | 2026087 | | |
| NMTN-03821 | SAV1885 | vraS | 2025225 | 2026268 | 2026097 | | |
| NMTN-05255 | SAV1885 | vraS | 2025225 | 2026268 | 2026219 | | |
| NMTN-15227 | SAV1886 | | 2026265 | 2026966 | 2026283 | − | conserved hypothetical protein |
| NMTN-11863 | SAV1886 | | 2026265 | 2026966 | 2026575 | | |
| NMTN-04899 | SAV1886 | | 2026265 | 2026966 | 2026708 | | |
| NMTN-02960 | SAV1886 | | 2026265 | 2026966 | 2026840 | | |
| NMTN-11651 | SAV1886 | | 2026265 | 2026966 | 2026840 | | |
| NMTN-06266 | SAV1886 | | 2026265 | 2026966 | 2026951 | | |
| NMTN-11882 | SAV1887 | | 2026981 | 2027367 | 2027257 | − | hypothetical protein |
| NMTN-08685 | SAV1887 | | 2026981 | 2027367 | 2027359 | | |
| NMTN-11839 | SAV1889 | | 2028873 | 2029859 | 2029101 | + | conserved hypothetical protein |
| NMTN-07265 | SAV1890 | | 2030290 | 2030742 | 2030486 | − | hypothetical protein |
| NMTN-07023 | SAV1890 | | 2030290 | 2030742 | 2030634 | | |
| NMTN-13487 | SAV1890 | | 2030290 | 2030742 | 2030665 | | |
| NMTN-06498 | SAV1893 | | 2033266 | 2033766 | 2033516 | + | hypothetical protein |
| NMTN-06497 | SAV1893 | | 2033266 | 2033766 | 2033518 | | |
| NMTN-06672 | SAV1893 | | 2033266 | 2033766 | 2033604 | | |
| NMTN-07152 | SAV1893 | | 2033266 | 2033766 | 2033637 | | |
| NMTN-03231 | SAV1894 | | 2034156 | 2034710 | 2034211 | + | hypothetical protein |
| NMTN-13545 | SAV1894 | | 2034156 | 2034710 | 2034358 | | |
| NMTN-09127 | SAV1895 | | 2034778 | 2035848 | 2035209 | − | hypothetical protein |
| NMTN-07823 | SAV1895 | | 2034778 | 2035848 | 2035817 | | |
| NMTN-10378 | SAV1896 | | 2036095 | 2036625 | 2036095 | − | conserved hypothetical protein |
| NMTN-04966 | SAV1896 | | 2036095 | 2036625 | 2036132 | | |
| NMTN-15178d | SAV1897 | | 2036795 | 2038156 | 2036934 | − | RNA methyltransferase homolog |
| NMTN-13475 | SAV1897 | | 2036795 | 2038156 | 2037145 | | |
| NMTN-00136 | SAV1897 | | 2036795 | 2038156 | 2037163 | | |
| NMTN-00137 | SAV1897 | | 2036795 | 2038156 | 2037163 | | |
| NMTN-11166 | SAV1897 | | 2036795 | 2038156 | 2037187 | | |
| NMTN-12187 | SAV1897 | | 2036795 | 2038156 | 2037234 | | |
| NMTN-10870 | SAV1897 | | 2036795 | 2038156 | 2037600 | | |
| NMTN-01468 | SAV1899 | | 2039952 | 2041379 | 2041338 | − | glutamyl-tRNAGln amidotransferase |
| | | | | | | | subunit B |
| NMTN-11673 | SAV1902 | putP | 2043520 | 2045058 | 2044268 | + | high affinity proline permease |
| NMTN-11997 | SAV1903 | | 2045147 | 2046346 | 2046334 | − | conserved hypothetical protein |
| NMTN-01622 | SAV1906 | pcrB | 2050555 | 2051247 | 2050607 | − | conserved hypothetical protein |
| NMTN-14046 | SAV1907 | | 2051419 | 2051721 | 2051549 | − | conserved hypothetical protein |
| NMTN-01592 | SAV1907 | | 2051419 | 2051721 | 2051613 | | |
| NMTN-10743 | SAV1908 | purB | 2051830 | 2053125 | 2051984 | − | adenylosuccinate lyase |
| NMTN-02875 | SAV1908 | purB | 2051830 | 2053125 | 2052463 | | |
| NMTN-04589 | SAV1908 | purB | 2051830 | 2053125 | 2052612 | | |
| NMTN-12131 | SAV1908 | purB | 2051830 | 2053125 | 2052862 | | |
| NMTN-05144 | SAV1908 | purB | 2051830 | 2053125 | 2052913 | | |
| NMTN-11242 | SAV1908 | purB | 2051830 | 2053125 | 2052966 | | |
| NMTN-14022 | SAV1909 | | 2053985 | 2055151 | 2054315 | + | staphopain |
| NMTN-06249 | SAV1909 | | 2053985 | 2055151 | 2054695 | | |
| NMTN-13520 | SAV1909 | | 2053985 | 2055151 | 2054709 | | |
| NMTN-09254 | SAV1909 | | 2053985 | 2055151 | 2054884 | | |
| NMTN-11243 | SAV1909 | | 2053985 | 2055151 | 2054915 | | |
| NMTN-13504 | SAV1911 | | 2055954 | 2056556 | 2056100 | − | conserved hypothetical protein |
| NMTN-04478 | SAV1911 | | 2055954 | 2056556 | 2056404 | | |
| NMTN-11957 | SAV1911 | | 2055954 | 2056556 | 2056409 | | |
| NMTN-02554 | SAV1914 | | 2059294 | 2060370 | 2059304 | + | nitric-oxide synthase homolog |
| NMTN-12727 | SAV1914 | | 2059294 | 2060370 | 2059333 | | |
| NMTN-12194 | SAV1914 | | 2059294 | 2060370 | 2059418 | | |
| NMTN-04134 | SAV1914 | | 2059294 | 2060370 | 2059439 | | |
| NMTN-06919 | SAV1914 | | 2059294 | 2060370 | 2059523 | | |
| NMTN-13840 | SAV1914 | | 2059294 | 2060370 | 2059610 | | |
| NMTN-13862 | SAV1914 | | 2059294 | 2060370 | 2059620 | | |
| NMTN-07497 | SAV1914 | | 2059294 | 2060370 | 2059857 | | |
| NMTN-00331 | SAV1914 | | 2059294 | 2060370 | 2059939 | | |
| NMTN-05355 | SAV1914 | | 2059294 | 2060370 | 2059939 | | |
| NMTN-09372 | SAV1915 | | 2060390 | 2061184 | 2060856 | + | hypothetical protein |
| NMTN-09372 | SAV1915 | | 2060390 | 2061184 | 2060856 | | |
| NMTN-09265 | SAV1916 | | 2061374 | 2062936 | 2061427 | − | hypothetical protein |
| NMTN-12923 | SAV1916 | | 2061374 | 2062936 | 2061508 | | |
| NMTN-10888 | SAV1916 | | 2061374 | 2062936 | 2061582 | | |
| NMTN-07449 | SAV1916 | | 2061374 | 2062936 | 2061675 | | |
| NMTN-09025 | SAV1916 | | 2061374 | 2062936 | 2061694 | | |
| NMTN-02250 | SAV1916 | | 2061374 | 2062936 | 2061735 | | |
| NMTN-06567 | SAV1916 | | 2061374 | 2062936 | 2062036 | | |
| NMTN-04631 | SAV1916 | | 2061374 | 2062936 | 2062284 | | |
| NMTN-00718 | SAV1916 | | 2061374 | 2062936 | 2062458 | | |
| NMTN-13282 | SAV1916 | | 2061374 | 2062936 | 2062540 | | |
| NMTN-03182 | SAV1917 | | 2063141 | 2064238 | 2063215 | − | conserved hypothetical protein |
| NMTN-12608 | SAV1917 | | 2063141 | 2064238 | 2063275 | | |
| NMTN-11064 | SAV1917 | | 2063141 | 2064238 | 2063317 | | |
| NMTN-13226 | SAV1917 | | 2063141 | 2064238 | 2063392 | | |
| NMTN-07922 | SAV1917 | | 2063141 | 2064238 | 2063716 | | |
| NMTN-02291 | SAV1917 | | 2063141 | 2064238 | 2063728 | | |
| NMTN-02518 | SAV1917 | | 2063141 | 2064238 | 2063799 | | |
| NMTN-06664 | SAV1917 | | 2063141 | 2064238 | 2064237 | | |
| NMTN-04769 | SAV1918 | | 2064607 | 2065167 | 2064865 | + | pyrazinamidase/nicotinamidase |
| | | | | | | | homolog |
| NMTN-10631 | SAV1918 | | 2064607 | 2065167 | 2064901 | | |
| NMTN-08020 | SAV1918 | | 2064607 | 2065167 | 2065002 | | |
| NMTN-10965 | SAV1919 | | 2065220 | 2066149 | 2066149 | + | manganese-dependent inorganic |
| | | | | | | | pyrophosphatase |
| NMTN-11459 | SAV1920 | aldH | 2066567 | 2067946 | 2066713 | + | aldehyde dehydrogenase |
| NMTN-04846 | SAV1922 | | 2069364 | 2069765 | 2069594 | + | hypothetical protein |
| NMTN-01723 | SAV1924 | | 2070338 | 2070511 | 2070408 | − | hypothetical protein |
| NMTN-05468 | SAV1925 | | 2070962 | 2072002 | 2071172 | + | hypothetical protein |
| NMTN-11893 | SAV1925 | | 2070962 | 2072002 | 2071649 | | |
| NMTN-11893 | SAV1925 | | 2070962 | 2072002 | 2071649 | | |
| NMTN-03437 | SAV1926 | | 2072059 | 2072901 | 2072393 | − | hypothetical protein |
| NMTN-06378 | SAV1926 | | 2072059 | 2072901 | 2072454 | | |
| NMTN-04883 | SAV1926 | | 2072059 | 2072901 | 2072620 | | |
| NMTN-13899 | SAV1926 | | 2072059 | 2072901 | 2072667 | | |
| NMTN-11080 | SAV1927 | | 2072898 | 2073455 | 2073179 | − | conserved hypothetical protein |
| NMTN-01449 | SAV1928 | | 2073515 | 2074039 | 2073597 | − | hypothetical protein |
| NMTN-12471 | SAV1929 | | 2074160 | 2074723 | 2074640 | + | hypothetical protein |
| NMTN-11264 | SAV1929 | | 2074160 | 2074723 | 2074641 | | |
| NMTN-01508 | SAV1932 | | 2076398 | 2077078 | 2076660 | − | hypothetical protein |
| NMTN-09076 | SAV1933 | | 2077079 | 2077975 | 2077096 | − | hypothetical protein |
| NMTN-12158 | SAV1933 | | 2077079 | 2077975 | 2077758 | | |
| NMTN-09620 | SAV1934 | | 2077972 | 2078352 | 2078082 | − | hypothetical protein |
| NMTN-06269 | SAV1934 | | 2077972 | 2078352 | 2078111 | | |
| NMTN-13110 | SAV1935 | | 2078610 | 2078786 | 2078723 | − | hypothetical protein |
| NMTN-02967 | SAV1936 | | 2079326 | 2080612 | 2079482 | + | hypothetical protein |
| NMTN-11293 | SAV1936 | | 2079326 | 2080612 | 2079872 | | |
| NMTN-11559 | SAV1938 | truncated- | 2079326 | 2081211 | 2080941 | − | truncated map-w protein |
| | | mapW | | | | | |
| NMTN-13870 | SAV1938 | truncated- | 2080891 | 2081211 | 2081008 | | |
| | | mapW | | | | | |
| NMTN-10907 | SAV1938 | truncated- | 2081196 | 2082626 | 2081488 | | |
| | | mapW | | | | | |
| NMTN-11729 | SAV1938 | truncated- | 2081196 | 2082626 | 2081506 | | |
| | | mapW | | | | | |
| NMTN-11145 | SAV1938 | truncated- | 2081196 | 2082626 | 2081600 | | |
| | | mapW | | | | | |
| NMTN-03882 | SAV1938 | truncated- | 2081196 | 2082626 | 2081649 | | |
| | | mapW | | | | | |
| NMTN-03945 | SAV1938 | truncated- | | | | | |
| | | mapW | | | | | |
| NMTN-13766 | SAV1938 | truncated- | 2081196 | 2082626 | 2081903 | | |
| | | mapW | | | | | |
| NMTN-04149 | SAV1938 | truncated- | 2081196 | 2082626 | 2081971 | | |
| | | mapW | | | | | |
| NMTN-05094 | SAV1938 | truncated- | 2081196 | 2082626 | 2082297 | | |
| | | mapW | | | | | |
| NMTN-09659 | SAV1938 | truncated- | 2081196 | 2082626 | 2082400 | | |
| | | mapW | | | | | |
| NMTN-10055 | SAV1939 | truncated- | 2082969 | 2083211 | 2083063 | + | truncated 6449-6450 beta-hemolysin |
| | | hlb | | | | | |
| NMTN-03698 | SAV1939 | truncated- | 2082969 | 2083211 | 2083168 | | |
| | | hlb | | | | | |
| NMTN-07384 | SAV1939 | truncated- | 2082969 | 2083211 | 2083191 | | |
| | | hlb | | | | | |
| NMTN-09316 | SAV1941 | | 2083921 | 2084094 | 2083991 | + | hypothetical protein |
| NMTN-12753 | SAV1941 | | 2083921 | 2084094 | 2084047 | | |
| NMTN-00001 | SAV1943 | truncated- | 2085246 | 2085713 | 2085371 | − | truncated amidase |
| | | lytA | | | | | |
| NMTN-12182 | SAV1943 | truncated- | 2085246 | 2085713 | 2085386 | | |
| | | lytA | | | | | |
| NMTN-02464 | SAV1944 | sak | 2086233 | 2086724 | 2086477 | − | staphylokinase precursor |
| NMTN-08802 | SAV1945 | | 2086939 | 2087670 | 2087082 | − | lytic enzyme |
| NMTN-06671 | SAV1945 | | 2086939 | 2087670 | 2087267 | | |
| NMTN-02996 | SAV1945 | | 2086939 | 2087670 | 2087617 | | |
| NMTN-14717 | SAV1948 | sep | 2088433 | 2089215 | 2088550 | − | enterotoxin P |
| NMTN-03552 | SAV1948 | sep | 2088433 | 2089215 | 2088687 | | |
| NMTN-08265 | SAV1949 | | 2089299 | 2089538 | 2089412 | + | hypothetical protein |
| NMTN-09512 | SAV1950 | | 2089579 | 2089953 | 2089965 | − | hypothetical protein |
| NMTN-04335 | SAV1951 | | 2090009 | 2090338 | 2090283 | − | hypothetical protein |
| NMTN-12961 | SAV1952 | | 2090342 | 2090494 | 2090358 | − | hypothetical protein |
| NMTN-11644 | SAV1953 | | 2090487 | 2094269 | 2090578 | − | phi PVL ORF 20 and 21 homologue |
| NMTN-13132 | SAV1953 | | 2090487 | 2094269 | 2091346 | | |
| NMTN-12627 | SAV1953 | | 2090487 | 2094269 | 2091486 | | |
| NMTN-05058 | SAV1953 | | 2090487 | 2094269 | 2091778 | | |
| NMTN-02627 | SAV1953 | | 2090487 | 2094269 | 2091953 | | |
| NMTN-06828t | SAV1953 | | 2090487 | 2094269 | 2092309 | | |
| NMTN-02578 | SAV1953 | | 2090487 | 2094269 | 2093156 | | |
| NMTN-04092 | SAV1953 | | 2090487 | 2094269 | 2093269 | | |
| NMTN-02902 | SAV1953 | | 2090487 | 2094269 | 2093272 | | |
| NMTN-08857 | SAV1953 | | 2090487 | 2094269 | 2093272 | | |
| NMTN-11272 | SAV1953 | | 2090487 | 2094269 | 2093488 | | |
| NMTN-13166 | SAV1953 | | 2090487 | 2094269 | 2093661 | | |
| NMTN-10930 | SAV1953 | | 2090487 | 2094269 | 2093749 | | |
| NMTN-12228 | SAV1953 | | 2090487 | 2094269 | 2093861 | | |
| NMTN-06872 | SAV1953 | | 2090487 | 2094269 | 2093990 | | |
| NMTN-09827 | SAV1953 | | 2090487 | 2094269 | 2094244 | | |
| NMTN-06389 | SAV1953 | | 2090487 | 2094269 | 2094266 | | |
| NMTN-06390 | SAV1954 | | 2094285 | 2095769 | 2094309 | − | hypothetical protein |
| NMTN-03922 | SAV1954 | | 2094285 | 2095769 | 2094584 | | |
| NMTN-13367 | SAV1954 | | 2094285 | 2095769 | 2094756 | | |
| NMTN-06538 | SAV1954 | | 2094285 | 2095769 | 2094821 | | |
| NMTN-12807 | SAV1954 | | 2094285 | 2095769 | 2094881 | | |
| NMTN-11767 | SAV1954 | | 2094285 | 2095769 | 2094904 | | |
| NMTN-01647 | SAV1954 | | 2094285 | 2095769 | 2095584 | | |
| NMTN-03245 | SAV1954 | | 2094285 | 2095769 | 2095645 | | |
| NMTN-11508 | SAV1954 | | 2094285 | 2095769 | 2095753 | | |
| NMTN-10886 | SAV1955 | | 2095766 | 2100295 | 2096063 | − | phi PVL ORF 15 and 16 homologue |
| NMTN-03844 | SAV1955 | | 2095766 | 2100295 | 2096140 | | |
| NMTN-12567 | SAV1955 | | 2095766 | 2100295 | 2096237 | | |
| NMTN-05090 | SAV1955 | | 2095766 | 2100295 | 2096274 | | |
| NMTN-12922 | SAV1955 | | 2095766 | 2100295 | 2096400 | | |
| NMTN-08082 | SAV1955 | | 2095766 | 2100295 | 2096999 | | |
| NMTN-07974 | SAV1955 | | 2095766 | 2100295 | 2098045 | | |
| NMTN-03766 | SAV1955 | | 2095766 | 2100295 | 2098326 | | |
| NMTN-12005 | SAV1955 | | 2095766 | 2100295 | 2098938 | | |
| NMTN-11125 | SAV1955 | | 2095766 | 2100295 | 2099645 | | |
| NMTN-11134 | SAV1955 | | 2095766 | 2100295 | 2099663 | | |
| NMTN-00355 | SAV1955 | | 2095766 | 2100295 | 2099956 | | |
| NMTN-06840d | SAV1956 | truncated | 2100364 | 2100711 | 2100435 | − | hypothetical protein |
| | | int | | | | | |
| NMTN-13772 | SAV1957 | | 2101206 | 2101850 | 2101209 | − | hypothetical protein |
| NMTN-04011 | SAV1957 | | 2101206 | 2101850 | 2101339 | | |
| NMTN-04496 | SAV1957 | | 2101206 | 2101850 | 2101804 | | |
| NMTN-04346 | SAV1958 | | 2101851 | 2102258 | 2101990 | − | hypothetical protein |
| NMTN-02206 | SAV1958 | | 2101851 | 2102258 | 2102065 | | |
| NMTN-11129 | SAV1958 | | 2101851 | 2102258 | 2102094 | | |
| NMTN-00548 | SAV1958 | | 2101851 | 2102258 | 2102209 | | |
| NMTN-06209 | SAV1959 | | 2102255 | 2102659 | 2102403 | − | hypothetical protein |
| NMTN-05637 | SAV1959 | | 2102255 | 2102659 | 2102521 | | |
| NMTN-13129 | SAV1960 | | 2102656 | 2103018 | 2102660 | − | hypothetical protein |
| NMTN-13157 | SAV1960 | | 2102656 | 2103018 | 2102660 | | |
| NMTN-12074 | SAV1960 | | 2102656 | 2103018 | 2102812 | | |
| NMTN-11198 | SAV1960 | | 2102656 | 2103018 | 2102898 | | |
| NMTN-09323 | SAV1960 | | 2102656 | 2103018 | 2102987 | | |
| NMTN-12540d | SAV1962 | | 2103276 | 2103560 | 2103483 | − | hypothetical protein |
| NMTN-11706 | SAV1963 | | 2103580 | 2104725 | 2103746 | − | hypothetical protein |
| NMTN-11652 | SAV1963 | | 2103580 | 2104725 | 2104031 | | |
| NMTN-02544 | SAV1963 | | 2103580 | 2104725 | 2104137 | | |
| NMTN-12660 | SAV1963 | | 2103580 | 2104725 | 2104340 | | |
| NMTN-11213 | SAV1964 | | 2104749 | 2105486 | 2104825 | − | hypothetical protein |
| NMTN-09161 | SAV1964 | | 2104749 | 2105486 | 2104828 | | |
| NMTN-11765 | SAV1964 | | 2104749 | 2105486 | 2105381 | | |
| NMTN-06327 | SAV1965 | | 2105470 | 2106657 | 2105873 | − | hypothetical protein |
| NMTN-13045 | SAV1965 | | 2105470 | 2106657 | 2106298 | | |
| NMTN-13473 | SAV1965 | | 2105470 | 2106657 | 2106597 | | |
| NMTN-06277 | SAV1966 | | 2106673 | 2108334 | 2106966 | − | hypothetical protein |
| NMTN-08860 | SAV1966 | | 2106673 | 2108334 | 2107280 | | |
| NMTN-06939 | SAV1966 | | 2106673 | 2108334 | 2108112 | | |
| NMTN-04147 | SAV1966 | | 2106673 | 2108334 | 2108249 | | |
| NMTN-07411 | SAV1966 | | 2106673 | 2108334 | 2108320 | | |
| NMTN-09536 | SAV1967 | | 2108331 | 2108675 | 2108335 | − | hypothetical protein |
| NMTN-04974 | SAV1967 | | 2108331 | 2108675 | 2108431 | | |
| NMTN-03672 | SAV1967 | | 2108331 | 2108675 | 2108603 | | |
| NMTN-03674 | SAV1967 | | 2108331 | 2108675 | 2108603 | | |
| NMTN-03131 | SAV1968 | | 2108805 | 2109104 | 2108948 | − | hypothetical protein |
| NMTN-03058 | SAV1968 | | 2108805 | 2109104 | 2109075 | | |
| NMTN-15248 | SAV1969 | | 2109336 | 2109752 | 2109388 | − | hypothetical protein |
| NMTN-13638 | SAV1969 | | 2109336 | 2109752 | 2109529 | | |
| NMTN-00613 | SAV1969 | | 2109336 | 2109752 | 2109555 | | |
| NMTN-00246 | SAV1970 | | 2109780 | 2109983 | 2109857 | − | hypothetical protein |
| NMTN-00311 | SAV1970 | | 2109780 | 2109983 | 2109857 | | |
| NMTN-11426 | SAV1974 | | 2110561 | 2111130 | 2110686 | − | dUTP pyrophosphatase |
| NMTN-03622 | SAV1974 | | 2110561 | 2111130 | 2110715 | | |
| NMTN-03628 | SAV1974 | | 2110561 | 2111130 | 2110715 | | |
| NMTN-06446 | SAV1976 | | 2111319 | 2111555 | 2111537 | − | hypothetical protein |
| NMTN-05162 | SAV1977 | | 2111548 | 2111913 | 2111805 | − | hypothetical protein |
| NMTN-00630 | SAV1977 | | 2111548 | 2111913 | 2111888 | | |
| NMTN-02679 | SAV1978 | | 2111928 | 2112170 | 2112082 | − | hypothetical protein |
| NMTN-11089 | SAV1980 | | 2112555 | 2112977 | 2112570 | − | hypothetical protein |
| NMTN-09359 | SAV1980 | | 2112555 | 2112977 | 2112685 | | |
| NMTN-12532 | SAV1980 | | 2112555 | 2112977 | 2112700 | | |
| NMTN-11768 | SAV1980 | | 2112555 | 2112977 | 2112709 | | |
| NMTN-00143 | SAV1980 | | 2112555 | 2112977 | 2112710 | | |
| NMTN-10871 | SAV1980 | | 2112555 | 2112977 | 2112821 | | |
| NMTN-12718 | SAV1980 | | 2112555 | 2112977 | 2112821 | | |
| NMTN-04158 | SAV1980 | | 2112555 | 2112977 | 2112864 | | |
| NMTN-04765 | SAV1980 | | 2112555 | 2112977 | 2112865 | | |
| NMTN-12958d | SAV1980 | | 2112555 | 2112977 | 2112867 | | |
| NMTN-09027 | SAV1980 | | 2112555 | 2112977 | 2112904 | | |
| NMTN-09203 | SAV1980 | | 2112555 | 2112977 | 2112904 | | |
| NMTN-12138 | SAV1981 | | 2112968 | 2113186 | 2112975 | − | hypothetical protein |
| NMTN-00050 | SAV1981 | | 2112968 | 2113186 | 2113040 | | |
| NMTN-02109 | SAV1982 | | 2113193 | 2114086 | 2113836 | − | hypothetical protein |
| NMTN-00349 | SAV1983 | | 2114116 | 2114586 | 2114427 | − | single-strand DNA-binding protein |
| NMTN-05258 | SAV1983 | | 2114116 | 2114586 | 2114438 | | |
| NMTN-12674 | SAV1984 | | 2114587 | 2115072 | 2114592 | − | hypothetical protein |
| NMTN-03200 | SAV1986 | | 2116207 | 2118150 | 2117088 | − | hypothetical protein |
| NMTN-13591 | SAV1986 | | 2116207 | 2118150 | 2117495 | | |
| NMTN-12926 | SAV1986 | | 2116207 | 2118150 | 2117522 | | |
| NMTN-06292 | SAV1986 | | 2116207 | 2118150 | 2117860 | | |
| NMTN-07923 | SAV1987 | | 2118159 | 2118422 | 2118349 | − | hypothetical protein |
| NMTN-06697 | SAV1991 | | 2119246 | 2119566 | 2119559 | − | hypothetical protein |
| NMTN-02683 | SAV1992 | | 2119618 | 2120253 | 2119728 | + | hypothetical protein |
| NMTN-01459 | SAV1992 | | 2119618 | 2120253 | 2120203 | | |
| NMTN-11341 | SAV1994 | | 2120652 | 2121407 | 2120670 | − | hypothetical protein |
| NMTN-03814 | SAV1994 | | 2120652 | 2121407 | 2120714 | | |
| NMTN-08536 | SAV1994 | | 2120652 | 2121407 | 2121138 | | |
| NMTN-15273 | SAV1994 | | 2120652 | 2121407 | 2121360 | | |
| NMTN-07174 | SAV2001 | | 2124619 | 2125083 | 2125001 | + | hypothetical protein |
| NMTN-09825 | SAV2001 | | 2124619 | 2125083 | 2125050 | | |
| NMTN-06967 | SAV2001 | | 2124619 | 2125083 | 2125066 | | |
| NMTN-13353 | SAV2002 | int | 2125142 | 2126179 | 2125215 | + | integrase |
| NMTN-09742 | SAV2002 | int | 2125142 | 2126179 | 2125595 | | |
| NMTN-07339 | SAV2002 | int | 2125142 | 2126179 | 2126059 | | |
| NMTN-10642 | SAV2003 | truncated- | 2126191 | 2127060 | 2126360 | + | truncated beta-hemplysin |
| | | hlb | | | | | |
| NMTN-06639 | SAV2003 | truncated- | 2126191 | 2127060 | 2126737 | | |
| | | hlb | | | | | |
| NMTN-04193 | SAV2003 | hlb | 2126191 | 2127060 | 2126740 | | |
| NMTN-09993 | SAV2003 | hlb | 2126191 | 2127060 | 2126820 | | |
| NMTN-08871 | SAV2003 | truncated- | 2126191 | 2127060 | 2127053 | | |
| | | hlb | | | | | |
| NMTN-02723 | SAV2004 | | 2127298 | 2128314 | 2127340 | − | hypothetical protein |
| NMTN-06289 | SAV2004 | | 2127298 | 2128314 | 2127364 | | |
| NMTN-07361 | SAV2004 | | 2127298 | 2128314 | 2127418 | | |
| NMTN-02595 | SAV2004 | | 2127298 | 2128314 | 2127767 | | |
| NMTN-02607 | SAV2004 | | 2127298 | 2128314 | 2127767 | | |
| NMTN-02608 | SAV2004 | | 2127298 | 2128314 | 2127767 | | |
| NMTN-03288 | SAV2004 | | 2127298 | 2128314 | 2127880 | | |
| NMTN-11907 | SAV2004 | | 2127298 | 2128314 | 2127880 | | |
| NMTN-11331 | SAV2005 | | 2128336 | 2129391 | 2128513 | − | hypothetical protein |
| NMTN-13229 | SAV2005 | | 2128336 | 2129391 | 2128661 | | |
| NMTN-13179 | SAV2005 | | 2128336 | 2129391 | 2129076 | | |
| NMTN-09440 | SAV2006 | | 2129827 | 2131029 | 2129954 | + | hypothetical protein |
| NMTN-07300 | SAV2006 | | 2129827 | 2131029 | 2130349 | | |
| NMTN-08720 | SAV2007 | | 2131337 | 2132644 | 2131514 | + | hypothetical protein |
| NMTN-11638 | SAV2007 | | 2131337 | 2132644 | 2131514 | | |
| NMTN-12820 | SAV2007 | | 2131337 | 2132644 | 2131830 | | |
| NMTN-03441 | SAV2007 | | 2131337 | 2132644 | 2132156 | | |
| NMTN-09347 | SAV2007 | | 2131337 | 2132644 | 2132160 | | |
| NMTN-11617 | SAV2007 | | 2131337 | 2132644 | 2132517 | | |
| NMTN-12603 | SAV2031 | | 2150992 | 2151735 | 2151422 | + | conserved hypothetical protein |
| NMTN-05419 | SAV2032 | | 2151760 | 2153007 | 2152460 | − | hypothetical protein |
| NMTN-01679 | SAV2033 | | 2153204 | 2153830 | 2153340 | + | conserved hypothetical protein |
| NMTN-01944 | SAV2033 | | 2153204 | 2153830 | 2153376 | | |
| NMTN-06409 | SAV2034 | | 2154191 | 2154976 | 2154530 | + | hypothetical protein |
| NMTN-01971 | SAV2034 | | 2154191 | 2154976 | 2154817 | | |
| NMTN-04484 | SAV2034 | | 2154191 | 2154976 | 2154935 | | |
| NMTN-06509 | SAV2036 | agrB | 2155946 | 2156509 | 2156008 | + | accessory gene regulator B |
| NMTN-04055 | SAV2036 | agrB | 2155946 | 2156509 | 2156088 | | |
| NMTN-12177 | SAV2038 | agrC | 2156853 | 2157968 | 2157912 | + | accessory gene regulator C |
| NMTN-03938 | SAV2039 | agrA | 2157987 | 2158703 | 2158059 | + | accessory gene regulator A |
| NMTN-09613 | SAV2040 | | 2159075 | 2160034 | 2159300 | − | hypothetical protein |
| NMTN-15189 | SAV2040 | | 2159075 | 2160034 | 2159644 | | |
| NMTN-03962 | SAV2040 | | 2159075 | 2160034 | 2159910 | | |
| NMTN-03868 | SAV2041 | scrB | 2160031 | 2161515 | 2160078 | − | sucrose-6-phosphate hydrolase |
| NMTN-06670 | SAV2041 | scrB | 2160031 | 2161515 | 2161140 | | |
| NMTN-02268 | SAV2042 | scrR | 2161664 | 2162614 | 2161701 | − | sucrose operon repressor |
| NMTN-12879 | SAV2042 | scrR | 2161664 | 2162614 | 2161918 | | |
| NMTN-06232 | SAV2042 | scrR | 2161664 | 2162614 | 2161987 | | |
| NMTN-05101 | SAV2042 | scrR | 2161664 | 2162614 | 2161990 | | |
| NMTN-12507 | SAV2042 | scrR | 2161664 | 2162614 | 2162045 | | |
| NMTN-06283 | SAV2043 | nrgA | 2162797 | 2164047 | 2162789 | − | probabale ammonium transporter |
| NMTN-14554 | SAV2043 | nrgA | 2162797 | 2164047 | 2162790 | | |
| NMTN-14468 | SAV2043 | nrgA | 2162797 | 2164047 | 2162829 | | |
| NMTN-09626 | SAV2043 | nrgA | 2162797 | 2164047 | 2162883 | | |
| NMTN-07745 | SAV2043 | nrgA | 2162797 | 2164047 | 2162917 | | |
| NMTN-07935 | SAV2043 | nrgA | 2162797 | 2164047 | 2163035 | | |
| NMTN-09965 | SAV2043 | nrgA | 2162797 | 2164047 | 2163077 | | |
| NMTN-02624 | SAV2043 | nrgA | 2162797 | 2164047 | 2163113 | | |
| NMTN-02532 | SAV2043 | nrgA | 2162797 | 2164047 | 2163215 | | |
| NMTN-00075 | SAV2043 | nrgA | 2162797 | 2164047 | 2163217 | | |
| NMTN-07406 | SAV2043 | nrgA | 2162797 | 2164047 | 2163298 | | |
| NMTN-13113 | SAV2043 | nrgA | 2162797 | 2164047 | 2163333 | | |
| NMTN-11607 | SAV2043 | nrgA | 2162797 | 2164047 | 2163408 | | |
| NMTN-04946 | SAV2043 | nrgA | 2162797 | 2164047 | 2163522 | | |
| NMTN-10012 | SAV2043 | ngrA | 2162797 | 2164047 | 2163531 | | |
| NMTN-11062d | SAV2043 | nrgA | 2162797 | 2164047 | 2164046 | | |
| NMTN-12435 | SAV2045 | | 2164539 | 2165618 | 2164747 | − | conserved hypothetical protein |
| NMTN-03968 | SAV2045 | | 2164539 | 2165618 | 2164827 | | |
| NMTN-07954 | SAV2045 | | 2164539 | 2165618 | 2165041 | | |
| NMTN-02872 | SAV2045 | | 2164539 | 2165618 | 2165059 | | |
| NMTN-04413 | SAV2045 | | 2164539 | 2165618 | 2165277 | | |
| NMTN-13217 | SAV2045 | | 2164539 | 2165618 | 2165304 | | |
| NMTN-13011 | SAV2045 | | 2164539 | 2165618 | 2165520 | | |
| NMTN-04397 | SAV2046 | | 2165921 | 2166556 | 2166497 | − | conserved hypothetical protein |
| NMTN-11087 | SAV2047 | vga | 2166809 | 2168737 | 2167179 | + | hypothetical ABC transporter |
| NMTN-02486 | SAV2047 | vga | 2166809 | 2168737 | 2167956 | | |
| NMTN-10273 | SAV2047 | vga | 2166809 | 2168737 | 2168331 | | |
| NMTN-10710 | SAV2048 | | 2169099 | 2170709 | 2169408 | + | hypothetical protein |
| NMTN-05103 | SAV2048 | | 2169099 | 2170709 | 2169469 | | |
| NMTN-08692 | SAV2048 | | 2169099 | 2170709 | 2169571 | | |
| NMTN-09927 | SAV2048 | | 2169099 | 2170709 | 2169812 | | |
| NMTN-06756 | SAV2048 | | 2169099 | 2170709 | 2170171 | | |
| NMTN-00752 | SAV2053 | ilvD | 2174094 | 2175782 | 2174203 | + | dihydroxy-acid dehydratase |
| NMTN-06663 | SAV2053 | ilvD | 2174094 | 2175782 | 2174441 | | |
| NMTN-11077 | SAV2053 | ilvD | 2174094 | 2175782 | 2174923 | | |
| NMTN-06290 | SAV2053 | ilvD | 2174094 | 2175782 | 2175442 | | |
| NMTN-11288 | SAV2054 | ilvB | 2175810 | 2177579 | 2176016 | + | acetolactate synthase large subunit |
| NMTN-08835d | SAV2054 | ilvB | 2175810 | 2177579 | 2176092 | | |
| NMTN-11983 | SAV2054 | ilvB | 2175810 | 2177579 | 2176588 | | |
| NMTN-10582 | SAV2054 | ilvB | 2175810 | 2177579 | 2176606 | | |
| NMTN-11886 | SAV2054 | ilvB | 2175810 | 2177579 | 2176783 | | |
| NMTN-02287 | SAV2054 | ilvB | 2175810 | 2177579 | 2177018 | | |
| NMTN-02664 | SAV2054 | ilvB | 2175810 | 2177579 | 2177493 | | |
| NMTN-10637 | SAV2054 | ilvB | 2175810 | 2177579 | 2177577 | | |
| NMTN-10637 | SAV2055 | | 2177579 | 2177833 | 2177577 | + | hypothetical protein |
| NMTN-10056 | SAV2055 | | 2177579 | 2177833 | 2177759 | | |
| NMTN-12517 | SAV2058 | leuB | 2180536 | 2181582 | 2180544 | + | 3-isopropylmalate dehydrogenase |
| NMTN-03592 | SAV2058 | leuB | 2180536 | 2181582 | 2180628 | | |
| NMTN-08475 | SAV2058 | leuB | 2180536 | 2181582 | 2181542 | | |
| NMTN-11921 | SAV2059 | leuC | 2181596 | 2182966 | 2182534 | + | 3-isopropylmalate dehydratase large |
| | | | | | | | subunit |
| NMTN-04802 | SAV2060 | leuD | 2182967 | 2183539 | 2182963 | + | 3-isopropylmalate dehydratase small |
| | | | | | | | subunit |
| NMTN-06357 | SAV2060 | leuD | 2182967 | 2183539 | 2183169 | | |
| NMTN-13106 | SAV2060 | leuD | 2182967 | 2183539 | 2183460 | | |
| NMTN-00820 | SAV2061 | ilvA | 2183554 | 2184822 | 2183698 | + | thereonine dehydratase |
| NMTN-11374 | SAV2061 | ilvA | 2183554 | 2184822 | 2183777 | | |
| NMTN-09079 | SAV2061 | ilvA | 2183554 | 2184822 | 2183932 | | |
| NMTN-03897 | SAV2061 | ilvA | 2183554 | 2184822 | 2183979 | | |
| NMTN-05169 | SAV2061 | ilvA | 2183554 | 2184822 | 2184345 | | |
| NMTN-11772 | SAV2061 | ilvA | 2183554 | 2184822 | 2184439 | | |
| NMTN-00010 | SAV2061 | ilvA | 2183554 | 2184822 | 2184780 | | |
| NMTN-12622 | SAV2061 | ilvA | 2183554 | 2184822 | 2184828 | | |
| NMTN-08635 | SAV2062 | | 2191620 | 2192075 | 2191620 | − | conserved hypothetical protein |
| NMTN-12383 | SAV2063 | | 2192068 | 2194218 | 2192273 | − | conserved hypothetical protein |
| NMTN-07484 | SAV2063 | | 2192068 | 2194218 | 2192421 | | |
| NMTN-04740 | SAV2063 | | 2192068 | 2194218 | 2192558 | | |
| NMTN-01890 | SAV2063 | | 2192068 | 2194218 | 2193488 | | |
| NMTN-08030 | SAV2063 | | 2192068 | 2194218 | 2193547 | | |
| NMTN-09958 | SAV2063 | | 2192068 | 2194218 | 2193848 | | |
| NMTN-11094 | SAV2063 | | 2192068 | 2194218 | 2193910 | | |
| NMTN-00789 | SAV2063 | | 2192068 | 2194218 | 2193912 | | |
| NMTN-08677 | SAV2063 | | 2192068 | 2194218 | 2194013 | | |
| NMTN-03805 | SAV2063 | | 2192068 | 2194218 | 2194034 | | |
| NMTN-11175 | SAV2063 | | 2192068 | 2194218 | 2194036 | | |
| NMTN-12693 | SAV2063 | | 2192068 | 2194218 | 2194078 | | |
| NMTN-10513 | SAV2063 | | 2192068 | 2194218 | 2194177 | | |
| NMTN-08891 | SAV2064 | sigB | 2194652 | 2195422 | 2194806 | − | sigma factor B |
| NMTN-05387 | SAV2064 | sigB | 2194652 | 2195422 | 2195088 | | |
| NMTN-13410 | SAV2064 | sigB | 2194652 | 2195422 | 2195129 | | |
| NMTN-04540 | SAV2064 | sigB | 2194652 | 2195422 | 2195280 | | |
| NMTN-07757 | SAV2065 | rsbW | 2195397 | 2195876 | 2195656 | − | anti-sigmaB factor |
| NMTN-06056 | SAV2066 | rsbV | 2195878 | 2196204 | 2195971 | − | anti-sigmaB factor antagonist |
| NMTN-09149 | SAV2066 | rsbV | 2195878 | 2196204 | 2196194 | | |
| NMTN-14501 | SAV2067 | rsbU | 2196323 | 2197324 | 2196644 | − | sigmaB regulation protein |
| NMTN-13666 | SAV2067 | rsbU | 2196323 | 2197324 | 2196999 | | |
| NMTN-13680 | SAV2067 | rsbU | 2196323 | 2197324 | 2197005 | | |
| NMTN-00527 | SAV2067 | rsbU | 2196323 | 2197324 | 2197086 | | |
| NMTN-11599 | SAV2067 | rsbU | 2196323 | 2197324 | 2197126 | | |
| NMTN-13280 | SAV2067 | rsbU | 2196323 | 2197324 | 2197167 | | |
| NMTN-15179 | SAV2068 | | 2197673 | 2198035 | 2197732 | − | conserved hypothetical protein |
| NMTN-12368 | SAV2068 | | 2197673 | 2198035 | 2197760 | | |
| NMTN-15331 | SAV2068 | | 2197673 | 2198035 | 2197767 | | |
| NMTN-04020 | SAV2068 | | 2197673 | 2198035 | 2197815 | | |
| NMTN-12505 | SAV2068 | | 2197673 | 2198035 | 2197815 | | |
| NMTN-03218 | SAV2068 | | 2197673 | 2198035 | 2197841 | | |
| NMTN-00556 | SAV2068 | | 2197673 | 2198035 | 2198012 | | |
| NMTN-08755 | SAV2068 | | 2197673 | 2198035 | 2198012 | | |
| NMTN-04819 | SAV2069 | | 2198032 | 2198202 | 2198059 | − | hypothetical protein |
| NMTN-09718 | SAV2069 | | 2198032 | 2198202 | 2198156 | | |
| NMTN-11585 | SAV2069 | | 2198032 | 2198202 | 2198204 | | |
| NMTN-14652 | SAV2069 | | 2198032 | 2198202 | 2198204 | | |
| NMTN-13708d | SAV2070 | alr | 2198287 | 2199435 | 2198594 | − | alanine racemase |
| NMTN-13683 | SAV2073 | | 2200342 | 2201925 | 2200488 | − | conserved hypothetical protein |
| NMTN-02995 | SAV2073 | | 2200342 | 2201925 | 2200964 | | |
| NMTN-04461 | SAV2073 | | 2200342 | 2201925 | 2201007 | | |
| NMTN-13104 | SAV2073 | | 2200342 | 2201925 | 2201027 | | |
| NMTN-11773 | SAV2073 | | 2200342 | 2201925 | 2201354 | | |
| NMTN-14407 | SAV2073 | | 2200342 | 2201925 | 2201686 | | |
| NMTN-03192 | SAV2074 | | 2201918 | 2202397 | 2202009 | − | hypothetical protein |
| NMTN-11874 | SAV2074 | | 2201918 | 2202397 | 2202012 | | |
| NMTN-10901 | SAV2074 | | 2201918 | 2202397 | 2202211 | | |
| NMTN-06506 | SAV2074 | | 2201918 | 2202397 | 2202299 | | |
| NMTN-05113 | SAV2075 | kdpC | 2202605 | 2203165 | 2202717 | − | probable potassium-transporting |
| | | | | | | | ATPase C chain |
| NMTN-09662 | SAV2075 | kdpC | 2202605 | 2203165 | 2203055 | | |
| NMTN-11944 | SAV2075 | kdpC | 2202605 | 2203165 | 2203125 | | |
| NMTN-02668 | SAV2076 | kdpB | 2203185 | 2205212 | 2203440 | − | probable potassium-transporting |
| | | | | | | | ATPase B chain |
| NMTN-08660 | SAV2076 | kdpB | 2203185 | 2205212 | 2204032 | | |
| NMTN-12406 | SAV2076 | kdpB | 2203185 | 2205212 | 2204035 | | |
| NMTN-12407 | SAV2076 | kdpB | 2203185 | 2205212 | 2204035 | | |
| NMTN-04652 | SAV2076 | kdpB | 2203185 | 2205212 | 2204280 | | |
| NMTN-12197 | SAV2076 | kdpB | 2203185 | 2205212 | 2204986 | | |
| NMTN-09039 | SAV2076 | kdpB | 2203185 | 2205212 | 2205037 | | |
| NMTN-03183 | SAV2077 | kdpA | 2205231 | 2206907 | 2205275 | − | probable potassium-transporting |
| | | | | | | | ATPase A chain |
| NMTN-10655 | SAV2077 | kdpA | 2205231 | 2206907 | 2205982 | | |
| NMTN-14531 | SAV2077 | kdpA | 2205231 | 2206907 | 2205982 | | |
| NMTN-12670 | SAV2077 | kdpA | 2205231 | 2206907 | 2206732 | | |
| NMTN-02877 | SAV2078 | kdpD | 2207179 | 2209836 | 2207737 | + | sensor protein |
| NMTN-11994 | SAV2078 | kdpD | 2207179 | 2209836 | 2207787 | | |
| NMTN-00593 | SAV2078 | kdpD | 2207179 | 2209836 | 2207998 | | |
| NMTN-06755 | SAV2078 | kdpD | 2207179 | 2209836 | 2208018 | | |
| NMTN-03512 | SAV2078 | kdpD | 2207179 | 2209836 | 2208026 | | |
| NMTN-12891 | SAV2078 | kdpD | 2207179 | 2209836 | 2208239 | | |
| NMTN-10869 | SAV2078 | kdpD | 2207179 | 2209836 | 2208338 | | |
| NMTN-07778 | SAV2078 | kdpD | 2207179 | 2209836 | 2208578 | | |
| NMTN-11808 | SAV2078 | kdpD | 2207179 | 2209836 | 2209291 | | |
| NMTN-04128 | SAV2078 | kdpD | 2207179 | 2209836 | 2209808 | | |
| NMTN-03701 | SAV2078 | kdpD | 2207179 | 2209836 | 2209831 | | |
| NMTN-00503 | SAV2078 | kdpD | 2207179 | 2209836 | 2209834 | | |
| NMTN-04094 | SAV2079 | kdpE | 2209836 | 2210531 | 2210425 | + | KDP operon transcriptional regulatory |
| | | | | | | | protein |
| NMTN-12590 | SAV2081 | | 2212403 | 2213923 | 2213013 | − | hypothetical protein |
| NMTN-09584 | SAV2086 | | 2218932 | 2219141 | 2218923 | − | hypothetical protein |
| NMTN-02828 | SAV2087 | | 2219153 | 2219446 | 2219232 | − | conserved hypothetical protein |
| NMTN-09428 | SAV2088 | | 2219612 | 2221096 | 2219621 | + | hypothetical protein |
| NMTN-03083 | SAV2088 | | 2219612 | 2221096 | 2219971 | | |
| NMTN-00196 | SAV2088 | | 2219612 | 2221096 | 2220113 | | |
| NMTN-14553 | SAV2088 | | 2219612 | 2221096 | 2220335 | | |
| NMTN-07540 | SAV2088 | | 2219612 | 2221096 | 2220637 | | |
| NMTN-06337 | SAV2088 | | 2219612 | 2221096 | 2220863 | | |
| NMTN-05295 | SAV2088 | | 2219612 | 2221096 | 2220897 | | |
| NMTN-10615 | SAV2091 | thiE | 2223542 | 2224183 | 2223872 | − | thiamin phosphate synthase |
| NMTN-10618 | SAV2091 | thiE | 2223542 | 2224183 | 2223872 | | |
| NMTN-11497 | SAV2091 | thiE | 2223542 | 2224183 | 2223883 | | |
| NMTN-12882 | SAV2092 | thiM | 2224185 | 2224976 | 2224960 | − | hydroxyethyl thiazole kinase |
| NMTN-12882 | SAV2093 | thiD | 2224960 | 2225790 | 2224960 | − | phosphomethylpyrimidine kinase |
| NMTN-08417 | SAV2093 | thiD | 2224960 | 2225790 | 2225073 | | |
| NMTN-11298 | SAV2093 | thiD | 2224960 | 2225790 | 2225144 | | |
| NMTN-11211 | SAV2093 | thiD | 2224960 | 2225790 | 2225558 | | |
| NMTN-01759 | SAV2093 | thiD | 2224960 | 2225790 | 2225561 | | |
| NMTN-01765 | SAV2093 | thiD | 2224960 | 2225790 | 2225561 | | |
| NMTN-01853 | SAV2093 | thiD | 2224960 | 2225790 | 2225561 | | |
| NMTN-01879 | SAV2093 | thiD | 2224960 | 2225790 | 2225561 | | |
| NMTN-01880 | SAV2093 | thiD | 2224960 | 2225790 | 2225561 | | |
| NMTN-01881 | SAV2093 | thiD | 2224960 | 2225790 | 2225561 | | |
| NMTN-01886 | SAV2093 | thiD | 2224960 | 2225790 | 2225561 | | |
| NMTN-01887 | SAV2093 | thiD | 2224960 | 2225790 | 2225561 | | |
| NMTN-01897 | SAV2093 | thiD | 2224960 | 2225790 | 2225561 | | |
| NMTN-01898 | SAV2093 | thiD | 2224960 | 2225790 | 2225561 | | |
| NMTN-01899 | SAV2093 | thiD | 2224960 | 2225790 | 2225561 | | |
| NMTN-01902 | SAV2093 | thiD | 2224960 | 2225790 | 2225561 | | |
| NMTN-01906 | SAV2093 | thiD | 2224960 | 2225790 | 2225561 | | |
| NMTN-01909 | SAV2093 | thiD | 2224960 | 2225790 | 2225561 | | |
| NMTN-01910 | SAV2093 | thiD | 2224960 | 2225790 | 2225561 | | |
| NMTN-01918 | SAV2093 | thiD | 2224960 | 2225790 | 2225561 | | |
| NMTN-01925 | SAV2093 | thiD | 2224960 | 2225790 | 2225561 | | |
| NMTN-01947 | SAV2093 | thiD | 2224960 | 2225790 | 2225561 | | |
| NMTN-01949 | SAV2093 | thiD | 2224960 | 2225790 | 2225561 | | |
| NMTN-01958 | SAV2093 | thiD | 2224960 | 2225790 | 2225561 | | |
| NMTN-01966 | SAV2093 | thiD | 2224960 | 2225790 | 2225561 | | |
| NMTN-01967 | SAV2093 | thiD | 2224960 | 2225790 | 2225561 | | |
| NMTN-01972 | SAV2093 | thiD | 2224960 | 2225790 | 2225561 | | |
| NMTN-01989 | SAV2093 | thiD | 2224960 | 2225790 | 2225561 | | |
| NMTN-02080 | SAV2093 | thiD | 2224960 | 2225790 | 2225561 | | |
| NMTN-02087 | SAV2093 | thiD | 2224960 | 2225790 | 2225561 | | |
| NMTN-02089 | SAV2093 | thiD | 2224960 | 2225790 | 2225561 | | |
| NMTN-02094 | SAV2093 | thiD | 2224960 | 2225790 | 2225561 | | |
| NMTN-02095 | SAV2093 | thiD | 2224960 | 2225790 | 2225561 | | |
| NMTN-02097 | SAV2093 | thiD | 2224960 | 2225790 | 2225561 | | |
| NMTN-02149 | SAV2093 | thiD | 2224960 | 2225790 | 2225561 | | |
| NMTN-02150 | SAV2093 | thiD | 2224960 | 2225790 | 2225561 | | |
| NMTN-02152 | SAV2093 | thiD | 2224960 | 2225790 | 2225561 | | |
| NMTN-02156 | SAV2093 | thiD | 2224960 | 2225790 | 2225561 | | |
| NMTN-02157 | SAV2093 | thiD | 2224960 | 2225790 | 2225561 | | |
| NMTN-02159 | SAV2093 | thiD | 2224960 | 2225790 | 2225561 | | |
| NMTN-02163 | SAV2093 | thiD | 2224960 | 2225790 | 2225561 | | |
| NMTN-02166 | SAV2093 | thiD | 2224960 | 2225790 | 2225561 | | |
| NMTN-02172 | SAV2093 | thiD | 2224960 | 2225790 | 2225561 | | |
| NMTN-02173 | SAV2093 | thiD | 2224960 | 2225790 | 2225561 | | |
| NMTN-02174 | SAV2093 | thiD | 2224960 | 2225790 | 2225561 | | |
| NMTN-02175 | SAV2093 | thiD | 2224960 | 2225790 | 2225561 | | |
| NMTN-02177 | SAV2093 | thiD | 2224960 | 2225790 | 2225561 | | |
| NMTN-02178 | SAV2093 | thiD | 2224960 | 2225790 | 2225561 | | |
| NMTN-02187 | SAV2093 | thiD | 2224960 | 2225790 | 2225561 | | |
| NMTN-02188 | SAV2093 | thiD | 2224960 | 2225790 | 2225561 | | |
| NMTN-02256 | SAV2093 | thiD | 2224960 | 2225790 | 2225561 | | |
| NMTN-02262 | SAV2093 | thiD | 2224960 | 2225790 | 2225561 | | |
| NMTN-02267 | SAV2093 | thiD | 2224960 | 2225790 | 2225561 | | |
| NMTN-02288 | SAV2093 | thiD | 2224960 | 2225790 | 2225561 | | |
| NMTN-02300 | SAV2093 | thiD | 2224960 | 2225790 | 2225561 | | |
| NMTN-02309 | SAV2093 | thiD | 2224960 | 2225790 | 2225561 | | |
| NMTN-02312 | SAV2093 | thiD | 2224960 | 2225790 | 2225561 | | |
| NMTN-02359 | SAV2093 | thiD | 2224960 | 2225790 | 2225561 | | |
| NMTN-02363 | SAV2093 | thiD | 2224960 | 2225790 | 2225561 | | |
| NMTN-02378 | SAV2093 | thiD | 2224960 | 2225790 | 2225561 | | |
| NMTN-02385 | SAV2093 | thiD | 2224960 | 2225790 | 2225561 | | |
| NMTN-02397 | SAV2093 | thiD | 2224960 | 2225790 | 2225561 | | |
| NMTN-02398 | SAV2093 | thiD | 2224960 | 2225790 | 2225561 | | |
| NMTN-02401 | SAV2093 | thiD | 2224960 | 2225790 | 2225561 | | |
| NMTN-02402 | SAV2093 | thiD | 2224960 | 2225790 | 2225561 | | |
| NMTN-02537 | SAV2093 | thiD | 2224960 | 2225790 | 2225561 | | |
| NMTN-02593 | SAV2093 | thiD | 2224960 | 2225790 | 2225561 | | |
| NMTN-02596 | SAV2093 | thiD | 2224960 | 2225790 | 2225561 | | |
| NMTN-02620 | SAV2093 | thiD | 2224960 | 2225790 | 2225561 | | |
| NMTN-02623 | SAV2093 | thiD | 2224960 | 2225790 | 2225561 | | |
| NMTN-02658 | SAV2093 | thiD | 2224960 | 2225790 | 2225561 | | |
| NMTN-02663 | SAV2093 | thiD | 2224960 | 2225790 | 2225561 | | |
| NMTN-02666 | SAV2093 | thiD | 2224960 | 2225790 | 2225561 | | |
| NMTN-02675 | SAV2093 | thiD | 2224960 | 2225790 | 2225561 | | |
| NMTN-02685 | SAV2093 | thiD | 2224960 | 2225790 | 2225561 | | |
| NMTN-02686 | SAV2093 | thiD | 2224960 | 2225790 | 2225561 | | |
| NMTN-02694 | SAV2093 | thiD | 2224960 | 2225790 | 2225561 | | |
| NMTN-02699 | SAV2093 | thiD | 2224960 | 2225790 | 2225561 | | |
| NMTN-02718 | SAV2093 | thiD | 2224960 | 2225790 | 2225561 | | |
| NMTN-02720 | SAV2093 | thiD | 2224960 | 2225790 | 2225561 | | |
| NMTN-02812 | SAV2093 | thiD | 2224960 | 2225790 | 2225561 | | |
| NMTN-02824 | SAV2093 | thiD | 2224960 | 2225790 | 2225561 | | |
| NMTN-02827 | SAV2093 | thiD | 2224960 | 2225790 | 2225561 | | |
| NMTN-02834 | SAV2093 | thiD | 2224960 | 2225790 | 2225561 | | |
| NMTN-02840 | SAV2093 | thiD | 2224960 | 2225790 | 2225561 | | |
| NMTN-03070 | SAV2093 | thiD | 2224960 | 2225790 | 2225561 | | |
| NMTN-03074 | SAV2093 | thiD | 2224960 | 2225790 | 2225561 | | |
| NMTN-03075 | SAV2093 | thiD | 2224960 | 2225790 | 2225561 | | |
| NMTN-03079 | SAV2093 | thiD | 2224960 | 2225790 | 2225561 | | |
| NMTN-03084 | SAV2093 | thiD | 2224960 | 2225790 | 2225561 | | |
| NMTN-03104 | SAV2093 | thiD | 2224960 | 2225790 | 2225561 | | |
| NMTN-03108 | SAV2093 | thiD | 2224960 | 2225790 | 2225561 | | |
| NMTN-03121 | SAV2093 | thiD | 2224960 | 2225790 | 2225561 | | |
| NMTN-03132 | SAV2093 | thiD | 2224960 | 2225790 | 2225561 | | |
| NMTN-03144 | SAV2093 | thiD | 2224960 | 2225790 | 2225561 | | |
| NMTN-03150 | SAV2093 | thiD | 2224960 | 2225790 | 2225561 | | |
| NMTN-03170 | SAV2093 | thiD | 2224960 | 2225790 | 2225561 | | |
| NMTN-03180 | SAV2093 | thiD | 2224960 | 2225790 | 2225561 | | |
| NMTN-03190 | SAV2093 | thiD | 2224960 | 2225790 | 2225561 | | |
| NMTN-03195 | SAV2093 | thiD | 2224960 | 2225790 | 2225561 | | |
| NMTN-03403 | SAV2093 | thiD | 2224960 | 2225790 | 2225561 | | |
| NMTN-03415 | SAV2093 | thiD | 2224960 | 2225790 | 2225561 | | |
| NMTN-03430 | SAV2093 | thiD | 2224960 | 2225790 | 2225561 | | |
| NMTN-03561 | SAV2093 | thiD | 2224960 | 2225790 | 2225561 | | |
| NMTN-03574 | SAV2093 | thiD | 2224960 | 2225790 | 2225561 | | |
| NMTN-03577 | SAV2093 | thiD | 2224960 | 2225790 | 2225561 | | |
| NMTN-03762 | SAV2093 | thiD | 2224960 | 2225790 | 2225561 | | |
| NMTN-03782 | SAV2093 | thiD | 2224960 | 2225790 | 2225561 | | |
| NMTN-03800 | SAV2093 | thiD | 2224960 | 2225790 | 2225561 | | |
| NMTN-03832 | SAV2093 | thiD | 2224960 | 2225790 | 2225561 | | |
| NMTN-03983 | SAV2093 | thiD | 2224960 | 2225790 | 2225561 | | |
| NMTN-03988 | SAV2093 | thiD | 2224960 | 2225790 | 2225561 | | |
| NMTN-03995 | SAV2093 | thiD | 2224960 | 2225790 | 2225561 | | |
| NMTN-05639 | SAV2093 | thiD | 2224960 | 2225790 | 2225561 | | |
| NMTN-11202 | SAV2093 | thiD | 2224960 | 2225790 | 2225561 | | |
| NMTN-13711 | SAV2093 | thiD | 2224960 | 2225790 | 2225774 | | |
| NMTN-05306 | SAV2094 | | 2225783 | 2226472 | 2225881 | − | hypothetical protein |
| NMTN-02677 | SAV2094 | | 2225783 | 2226472 | 2226089 | | |
| NMTN-00834 | SAV2094 | | 2225783 | 2226472 | 2226122 | | |
| NMTN-00426 | SAV2094 | | 2225783 | 2226472 | 2226325 | | |
| NMTN-06531 | SAV2096 | | 2227943 | 2228338 | 2228057 | − | hypothetical protein |
| NMTN-05459 | SAV2096 | | 2227943 | 2228338 | 2228060 | | |
| NMTN-02495 | SAV2096 | | 2227943 | 2228338 | 2228062 | | |
| NMTN-09009 | SAV2096 | | 2227943 | 2228338 | 2228335 | | |
| NMTN-02126 | SAV2101 | | 2230882 | 2231115 | 2231040 | − | conserved hypothetical protein |
| NMTN-02208 | SAV2104 | atpG | 2233535 | 2234401 | 2234066 | − | ATP synthase gamma chain |
| NMTN-08119 | SAV2111 | mnaA | 2238744 | 2239874 | 2238821 | − | UDP-GlcNAc 2-epimerase |
| NMTN-00209 | SAV2111 | mnaA | 2238744 | 2239874 | 2238863 | | |
| NMTN-14642 | SAV2111 | mnaA | 2238744 | 2239874 | 2239850 | | |
| NMTN-03277 | SAV2112 | upp | 2239895 | 2240524 | 2240127 | − | uracil phosphoribosyl transferase |
| NMTN-09033 | SAV2113 | glyA | 2240552 | 2241790 | 2241713 | − | serine hydroxymethyl transferase |
| NMTN-08850 | SAV2114 | | 2241817 | 2242341 | 2241924 | − | conserved hypothetical protein |
| NMTN-11642 | SAV2114 | | 2241817 | 2242341 | 2241983 | | |
| NMTN-03186 | SAV2115 | | 2242448 | 2242867 | 2242537 | − | hypothetical protein |
| NMTN-03191 | SAV2115 | | 2242448 | 2242867 | 2242537 | | |
| NMTN-05055 | SAV2117 | | 2243994 | 2244830 | 2244032 | − | hypothetical protein |
| NMTN-03531 | SAV2117 | | 2243994 | 2244830 | 2244271 | | |
| NMTN-08127 | | tdk | 2245894 | 2246493 | 2246234 | | |
| NMTN-10641 | SAV2121 | rho | 2247212 | 2248528 | 2247475 | − | transcription termination factor Rho |
| NMTN-04809 | SAV2121 | rho | 2247212 | 2248528 | 2247660 | | |
| NMTN-10653 | SAV2121 | rho | 2247212 | 2248528 | 2247705 | | |
| NMTN-07423 | SAV2121 | rho | 2247212 | 2248528 | 2247780 | | |
| NMTN-02746 | SAV2121 | rho | 2247212 | 2248528 | 2247859 | | |
| NMTN-04536 | SAV2121 | rho | 2247212 | 2248528 | 2248263 | | |
| NMTN-08915 | SAV2121 | rho | 2247212 | 2248528 | 2248312 | | |
| NMTN-04296 | SAV2121 | rho | 2247212 | 2248528 | 2248318 | | |
| NMTN-04684 | SAV2121 | rho | 2247212 | 2248528 | 2248318 | | |
| NMTN-01625 | SAV2121 | rho | 2247212 | 2248528 | 2248327 | | |
| NMTN-13781 | SAV2121 | rho | 2247212 | 2248528 | 2248327 | | |
| NMTN-11444 | SAV2121 | rho | 2247212 | 2248528 | 2248350 | | |
| NMTN-09187 | SAV2121 | rho | 2247212 | 2248528 | 2248384 | | |
| NMTN-03903 | SAV2122 | | 2248775 | 2250202 | 2249033 | − | hypothetical protein |
| NMTN-12558 | SAV2122 | | 2248775 | 2250202 | 2249772 | | |
| NMTN-07480 | SAV2123 | | 2250440 | 2250778 | 2250697 | − | conserved hypothetical protein |
| NMTN-13125 | SAV2123 | | 2250440 | 2250778 | 2250736 | | |
| NMTN-01548 | SAV2124 | murZ | 2250864 | 2252123 | 2251399 | − | UDP-N-acetylglucosamine 1- |
| | | | | | | | carboxylvinyl transferase 2 |
| NMTN-01549 | SAV2124 | murZ | 2250864 | 2252123 | 2251399 | | |
| NMTN-01588 | SAV2124 | murZ | 2250864 | 2252123 | 2251399 | | |
| NMTN-01605 | SAV2124 | murZ | 2250864 | 2252123 | 2251399 | | |
| NMTN-04316 | SAV2124 | murZ | 2250864 | 2252123 | 2251401 | | |
| NMTN-04714 | SAV2124 | murZ | 2250864 | 2252123 | 2251541 | | |
| NMTN-01447 | SAV2129 | | 2256885 | 2257745 | 2257371 | − | hypothetical protein |
| NMTN-00608 | SAV2129 | | 2256885 | 2257745 | 2257634 | | |
| NMTN-09408 | SAV2131 | | 2259104 | 2259775 | 2259446 | − | hypothetical protein |
| NMTN-08895 | SAV2132 | | 2260174 | 2261367 | 2260319 | − | hypothetical protein |
| NMTN-11744 | SAV2132 | | 2260174 | 2261367 | 2260615 | | |
| NMTN-14419 | SAV2132 | | 2260174 | 2261367 | 2261243 | | |
| NMTN-11140 | SAV2132 | | 2260174 | 2261367 | 2261287 | | |
| NMTN-09186 | SAV2133 | hmrA | 2261367 | 2262551 | 2261672 | − | HmrA |
| NMTN-01579 | SAV2133 | hmrA | 2261367 | 2262551 | 2261798 | | |
| NMTN-07765 | SAV2133 | hmrA | 2261367 | 2262551 | 2262132 | | |
| NMTN-03816 | SAV2133 | hmrA | 2261367 | 2262551 | 2262501 | | |
| NMTN-15294 | SAV2134 | luxS | 2262862 | 2263332 | 2263196 | + | autoinducer-2 production protein |
| NMTN-11493 | SAV2135 | | 2263484 | 2263834 | 2263615 | − | conserved hypothetical protein |
| NMTN-11538 | SAV2135 | | 2263484 | 2263834 | 2263825 | | |
| NMTN-00230 | SAV2136 | pdp | 2263848 | 2265188 | 2264555 | − | pyrimidine nucleoside phosphorylase |
| NMTN-11366 | SAV2136 | pdp | 2263848 | 2265188 | 2264555 | | |
| NMTN-04729 | SAV2136 | pdp | 2263848 | 2265188 | 2264696 | | |
| NMTN-03771 | SAV2137 | | 2265428 | 2266090 | 2265459 | − | deoxyribose-phosphate aldolase |
| NMTN-04697 | SAV2137 | | 2265428 | 2266090 | 2265940 | | |
| NMTN-08131 | SAV2138 | deoD | 2266404 | 2267114 | 2266461 | + | purine nucleoside phosphorylase |
| NMTN-00644 | SAV2138 | deoD | 2266404 | 2267114 | 2266860 | | |
| NMTN-02008 | SAV2138 | deoD | 2266404 | 2267114 | 2266974 | | |
| NMTN-11386 | SAV2138 | deoD | 2266404 | 2267114 | 2267100 | | |
| NMTN-13004d | SAV2142 | | 2270793 | 2271023 | 2270921 | − | hypothetical protein |
| NMTN-11342 | SAV2143 | | 2271054 | 2271992 | 2271470 | − | hypothetical protein |
| NMTN-00297 | SAV2143 | | 2271054 | 2271992 | 2271986 | | |
| NMTN-01875 | SAV2144 | | 2272428 | 2273093 | 2273057 | − | conserved hypothetical protein |
| NMTN-13492 | SAV2145 | czrA | 2273245 | 2273565 | 2273513 | + | repressor protein |
| NMTN-13346 | SAV2145 | czrA | 2273245 | 2273565 | 2273530 | | |
| NMTN-00173 | SAV2146 | czrB | 2273567 | 2274544 | 2273664 | + | cation-efflux system membrane protein |
| | | | | | | | homolog |
| NMTN-14517 | SAV2146 | czrB | 2273567 | 2274544 | 2274029 | | |
| NMTN-02876 | SAV2146 | czrB | 2273567 | 2274544 | 2274236 | | |
| NMTN-09812 | SAV2146 | czrB | 2273567 | 2274544 | 2274382 | | |
| NMTN-01487 | SAV2146 | czrB | 2273567 | 2274544 | 2274429 | | |
| NMTN-04069 | SAV2147 | | 2274810 | 2275901 | 2274937 | + | lytic regulatory protein truncated with |
| | | | | | | | Tn554 |
| NMTN-09622 | SAV2147 | | 2274810 | 2275901 | 2275132 | | |
| NMTN-01507 | SAV2147 | | 2274810 | 2275901 | 2275294 | | |
| NMTN-14662 | SAV2152 | | 2277934 | 2278791 | 2278055 | − | conserved hypothetical protein |
| NMTN-12714 | SAV2152 | | 2277934 | 2278791 | 2278280 | | |
| NMTN-06880d | SAV2153 | | 2278859 | 2279641 | 2279091 | − | hypothetical protein |
| NMTN-09672 | SAV2153 | | 2278859 | 2279641 | 2279379 | | |
| NMTN-11623d | SAV2153 | | 2278859 | 2279641 | 2279403 | | |
| NMTN-08466 | SAV2153 | | 2278859 | 2279641 | 2279523 | | |
| NMTN-09777 | SAV2153 | | 2278859 | 2279641 | 2279631 | | |
| NMTN-01551 | SAV2156 | mtlF | 2282359 | 2283897 | 2282419 | + | mannitol specific IIBC component of |
| | | | | | | | PTS system |
| NMTN-03006 | SAV2156 | mtlF | 2282359 | 2283897 | 2282647 | | |
| NMTN-05248 | SAV2156 | mtlF | 2282359 | 2283897 | 2283437 | | |
| NMTN-08676 | SAV2157 | | 2283932 | 2286064 | 2284357 | + | hypothetical protein |
| NMTN-15337 | SAV2157 | | 2283932 | 2286064 | 2284913 | | |
| NMTN-04123 | SAV2157 | | 2283932 | 2286064 | 2284934 | | |
| NMTN-11023 | SAV2157 | | 2283932 | 2286064 | 2284996 | | |
| NMTN-12774 | SAV2158 | mtlA | 2286076 | 2286510 | 2286392 | + | PTS system, mannitol specific IIA |
| | | | | | | | component |
| NMTN-05401 | SAV2159 | mtlD | 2286510 | 2287616 | 2287097 | + | mannitol-1-phosphate 5-dehydrogenase |
| NMTN-09210 | SAV2159 | mtlD | 2286510 | 2287616 | 2287302 | | |
| NMTN-06251 | SAV2159 | mtlD | 2286510 | 2287616 | 2287409 | | |
| NMTN-09420 | SAV2159 | mtlD | 2286510 | 2287616 | 2287419 | | |
| NMTN-09280 | SAV2160 | fmtB(mrp) | 2287935 | 2295380 | 2287990 | − | FmtB protein |
| NMTN-04354 | SAV2160 | fmtB(mrp) | 2287935 | 2295380 | 2288641 | | |
| NMTN-11254 | SAV2160 | fmtB(mrp) | 2287935 | 2295380 | 2288708 | | |
| NMTN-07408 | SAV2160 | fmtB(mrp) | 2287935 | 2295380 | 2289189 | | |
| NMTN-07395 | SAV2160 | fmtB(mrp) | 2287935 | 2295380 | 2289200 | | |
| NMTN-07398 | SAV2160 | fmtB(mrp) | 2287935 | 2295380 | 2289200 | | |
| NMTN-14699 | SAV2160 | fmtB(mrp) | 2287935 | 2295380 | 2289653 | | |
| NMTN-00057 | SAV2160 | fmtB(mrp) | 2287935 | 2295380 | 2290443 | | |
| NMTN-10662 | SAV2160 | fmtB(mrp) | 2287935 | 2295380 | 2290760 | | |
| NMTN-09935 | SAV2160 | fmtB(mrp) | 2287935 | 2295380 | 2290800 | | |
| NMTN-02138 | SAV2160 | fmtB(mrp) | 2287935 | 2295380 | 2291860 | | |
| NMTN-14507 | SAV2160 | fmtB(mrp) | 2287935 | 2295380 | 2292229 | | |
| NMTN-06206 | SAV2160 | fmtB(mrp) | 2287935 | 2295380 | 2292317 | | |
| NMTN-14565 | SAV2160 | fmtB(mrp) | 2287935 | 2295380 | 2292652 | | |
| NMTN-06719 | SAV2160 | fmtB(mrp) | 2287935 | 2295380 | 2292728 | | |
| NMTN-11679 | SAV2160 | fmtB(mrp) | 2287935 | 2295380 | 2293214 | | |
| NMTN-06152 | SAV2160 | fmtB(mrp) | 2287935 | 2295380 | 2293585 | | |
| NMTN-06158 | SAV2160 | fmtB(mrp) | 2287935 | 2295380 | 2293585 | | |
| NMTN-09670 | SAV2160 | fmtB(mrp) | 2287935 | 2295380 | 2294043 | | |
| NMTN-13098 | SAV2160 | fmtB(mrp) | 2287935 | 2295380 | 2294151 | | |
| NMTN-04909 | SAV2160 | fmtB(mrp) | 2287935 | 2295380 | 2294324 | | |
| NMTN-11008 | SAV2160 | fmtB(mrp) | 2287935 | 2295380 | 2294576 | | |
| NMTN-14053 | SAV2160 | fmtB(mrp) | 2287935 | 2295380 | 2294777 | | |
| NMTN-09330 | SAV2161 | glmM | 2295658 | 2297013 | 2296079 | − | phosphoglucosamine-mutase |
| | | (femD) | | | | | |
| NMTN-09505 | SAV2162 | | 2297040 | 2297972 | 2297051 | − | conserved hypothetical protein |
| NMTN-11545 | SAV2162 | | 2297040 | 2297972 | 2297099 | | |
| NMTN-13821 | SAV2162 | | 2297040 | 2297972 | 2297658 | | |
| NMTN-10979 | SAV2164 | arg | 2298972 | 2299880 | 2298977 | − | arginase |
| NMTN-09246 | SAV2164 | arg | 2298972 | 2299880 | 2299853 | | |
| NMTN-01990 | | rRNA-23S | | | 2300935 | | |
| NMTN-02023 | | rRNA-23S | | | 2302735 | | |
| NMTN-01955 | | rRNA-16S | | | 2305514 | | |
| NMTN-10407 | SAV2166 | | 2307333 | 2308775 | 2307345 | − | hypothetical protein |
| NMTN-04726 | SAV2166 | | 2307333 | 2308775 | 2307346 | | |
| NMTN-09240 | SAV2166 | | 2307333 | 2308775 | 2308096 | | |
| NMTN-01617 | SAV2166 | | 2307333 | 2308775 | 2308161 | | |
| NMTN-14530 | SAV2166 | | 2307333 | 2308775 | 2308605 | | |
| NMTN-08740 | SAV2166 | | 2307333 | 2308775 | 2308683 | | |
| NMTN-10377 | SAV2167 | | 2309098 | 2309571 | 2309114 | − | hypothetical protein |
| NMTN-07474 | SAV2167 | | 2309098 | 2309571 | 2309136 | | |
| NMTN-00329 | SAV2167 | | 2309098 | 2309571 | 2309202 | | |
| NMTN-03220 | SAV2167 | | 2309098 | 2309571 | 2309410 | | |
| NMTN-05118 | SAV2167 | | 2309098 | 2309571 | 2309448 | | |
| NMTN-03603 | SAV2167 | | 2309098 | 2309571 | 2309525 | | |
| NMTN-03228 | SAV2168 | | 2309670 | 2310191 | 2309673 | − | putative multidrug transporter |
| NMTN-01945 | SAV2168 | | 2309670 | 2310191 | 2310017 | | |
| NMTN-03831 | SAV2169 | | 2310155 | 2310814 | 2310155 | − | hypothetical protein |
| NMTN-12451 | SAV2169 | | 2310155 | 2310814 | 2310337 | | |
| NMTN-14595 | SAV2169 | | 2310155 | 2310814 | 2310357 | | |
| NMTN-03806 | SAV2169 | | 2310155 | 2310814 | 2310523 | | |
| NMTN-02101 | SAV2169 | | 2310155 | 2310814 | 2310534 | | |
| NMTN-07951 | SAV2170 | | 2310970 | 2311656 | 2311460 | − | hypothetical protein |
| NMTN-04933 | SAV2170 | | 2310970 | 2311656 | 2311496 | | |
| NMTN-12401 | SAV2170 | | 2310970 | 2311656 | 2311655 | | |
| NMTN-03929 | SAV2171 | | 2311672 | 2312859 | 2311799 | − | hypothetical protein |
| NMTN-09215 | SAV2171 | | 2311672 | 2312859 | 2312087 | | |
| NMTN-07502 | SAV2171 | | 2311672 | 2312859 | 2312475 | | |
| NMTN-08769 | SAV2171 | | 2311672 | 2312859 | 2312724 | | |
| NMTN-12456 | SAV2172 | | 2312903 | 2313418 | 2312932 | − | conserved hypothetical protein |
| NMTN-13381 | SAV2172 | | 2312903 | 2313418 | 2312937 | | |
| NMTN-13416 | SAV2172 | | 2312903 | 2313418 | 2312966 | | |
| NMTN-04558 | SAV2172 | | 2312903 | 2313418 | 2313310 | | |
| NMTN-00602 | SAV2174 | | 2313924 | 2315294 | 2313959 | − | conserved hypothetical protein |
| NMTN-07383 | SAV2174 | | 2313924 | 2315294 | 2314113 | | |
| NMTN-11690 | SAV2174 | | 2313924 | 2315294 | 2314129 | | |
| NMTN-00414 | SAV2174 | | 2313924 | 2315294 | 2314684 | | |
| NMTN-06566 | SAV2174 | | 2313924 | 2315294 | 2314762 | | |
| NMTN-11619d | SAV2174 | | 2313924 | 2315294 | 2314868 | | |
| NMTN-06675 | SAV2174 | | 2313924 | 2315294 | 2315056 | | |
| NMTN-06676 | SAV2174 | | 2313924 | 2315294 | 2315056 | | |
| NMTN-06356 | SAV2174 | | 2313924 | 2315294 | 2315129 | | |
| NMTN-03244 | SAV2174 | | 2313924 | 2315294 | 2315242 | | |
| NMTN-02878 | SAV2175 | | 2315530 | 2316498 | 2315948 | − | hypothetical protein |
| NMTN-02884 | SAV2175 | | 2315530 | 2316498 | 2315948 | | |
| NMTN-08117 | SAV2175 | | 2315530 | 2316498 | 2316019 | | |
| NMTN-03504 | SAV2175 | | 2315530 | 2316498 | 2316041 | | |
| NMTN-04180 | SAV2175 | | 2315530 | 2316498 | 2316317 | | |
| NMTN-09698 | SAV2175 | | 2315530 | 2316498 | 2316388 | | |
| NMTN-09717 | SAV2175 | | 2315530 | 2316498 | 2316388 | | |
| NMTN-05526 | SAV2176 | | 2316495 | 2317526 | 2316608 | − | hypothetical protein |
| NMTN-06607 | SAV2176 | | 2316495 | 2317526 | 2316755 | | |
| NMTN-10535 | SAV2176 | | 2316495 | 2317526 | 2317007 | | |
| NMTN-07448 | SAV2176 | | 2316495 | 2317526 | 2317109 | | |
| NMTN-09257 | SAV2176 | | 2316495 | 2317526 | 2317131 | | |
| NMTN-08990 | SAV2177 | | 2317538 | 2318521 | 2317875 | − | hypothetical protein |
| NMTN-06699 | SAV2177 | | 2317538 | 2318521 | 2317880 | | |
| NMTN-12756 | SAV2178 | | 2318913 | 2319983 | 2318990 | − | conserved hypothetical protein |
| NMTN-13716 | SAV2178 | | 2318913 | 2319983 | 2319154 | | |
| NMTN-00049 | SAV2178 | | 2318913 | 2319983 | 2319771 | | |
| NMTN-12545 | SAV2179 | | 2319990 | 2321747 | 2321548 | − | conserved hypothetical protein |
| NMTN-12547 | SAV2179 | | 2319990 | 2321747 | 2321548 | | |
| NMTN-12559 | SAV2179 | | 2319990 | 2321747 | 2321548 | | |
| NMTN-11814 | SAV2180 | | 2321734 | 2322927 | 2321813 | − | hypothetical protein |
| NMTN-00252 | SAV2180 | | 2321734 | 2322927 | 2322248 | | |
| NMTN-05624 | SAV2180 | | 2321734 | 2322927 | 2322393 | | |
| NMTN-02896 | SAV2180 | | 2321734 | 2322927 | 2322402 | | |
| NMTN-11608 | SAV2180 | | 2321734 | 2322927 | 2322506 | | |
| NMTN-08977 | SAV2181 | | 2323029 | 2325005 | 2323538 | + | hypothetical protein |
| NMTN-11381 | SAV2181 | | 2323029 | 2325005 | 2323879 | | |
| NMTN-11653 | SAV2181 | | 2323029 | 2325005 | 2324342 | | |
| NMTN-10198 | SAV2181 | | 2323029 | 2325005 | 2324504 | | |
| NMTN-11306 | SAV2184 | | 2325990 | 2326526 | 2326348 | − | hypothetical protein |
| NMTN-03969 | SAV2185 | | 2326701 | 2328263 | 2327048 | − | glycine betaine transporter opuD |
| | | | | | | | homolog |
| NMTN-02520 | SAV2185 | | 2326701 | 2328263 | 2327464 | | |
| NMTN-09843 | SAV2185 | | 2326701 | 2328263 | 2327479 | | |
| NMTN-14647 | SAV2185 | | 2326701 | 2328263 | 2327486 | | |
| NMTN-07372 | SAV2185 | | 2326701 | 2328263 | 2327765 | | |
| NMTN-10709 | SAV2185 | | 2326701 | 2328263 | 2327820 | | |
| NMTN-06255 | SAV2186 | | 2328571 | 2329578 | 2328820 | − | hypothetical protein |
| NMTN-11621 | SAV2186 | | 2328571 | 2329578 | 2328884 | | |
| NMTN-10701 | SAV2186 | | 2328571 | 2329578 | 2329345 | | |
| NMTN-00377 | SAV2187 | | 2329813 | 2330814 | 2329989 | − | hypothetical protein |
| NMTN-00323 | SAV2187 | | 2329813 | 2330814 | 2330033 | | |
| NMTN-11314 | SAV2187 | | 2329813 | 2330814 | 2330039 | | |
| NMTN-00615 | SAV2187 | | 2329813 | 2330814 | 2330709 | | |
| NMTN-11943 | SAV2188 | | 2331092 | 2331961 | 2331221 | − | conserved hypothetical protein |
| NMTN-05259 | SAV2188 | | 2331092 | 2331961 | 2331340 | | |
| NMTN-04401 | SAV2188 | | 2331092 | 2331961 | 2331396 | | |
| NMTN-10622 | SAV2189 | lacG | 2332227 | 2333639 | 2332411 | − | 6-phospho-beta-galactosidase |
| NMTN-15318d | SAV2189 | lacG | 2332227 | 2333639 | 2332446 | | |
| NMTN-08395 | SAV2189 | lacG | 2332227 | 2333639 | 2332808 | | |
| NMTN-04314 | SAV2189 | lacG | 2332227 | 2333639 | 2332886 | | |
| NMTN-14399 | SAV2189 | lacG | 2332227 | 2333639 | 2333132 | | |
| NMTN-05243 | SAV2189 | lacG | 2332227 | 2333639 | 2333358 | | |
| NMTN-09001 | SAV2190 | lacE | 2333657 | 2335369 | 2333778 | − | PTS system, lactose-specific IIBC |
| | | | | | | | component |
| NMTN-11521 | SAV2190 | lacE | 2333657 | 2335369 | 2333991 | | |
| NMTN-08745 | SAV2190 | lacE | 2333657 | 2335369 | 2334180 | | |
| NMTN-13796d | SAV2190 | lacE | 2333657 | 2335369 | 2334841 | | |
| NMTN-06472 | SAV2190 | lacE | 2333657 | 2335369 | 2335303 | | |
| NMTN-11685 | SAV2190 | lacE | 2333657 | 2335369 | 2335305 | | |
| NMTN-06631 | SAV2191 | lacF | 2335375 | 2335686 | 2335691 | − | PTS system, lactose-specific IIA |
| | | | | | | | component |
| NMTN-03563 | SAV2192 | lacD | 2335708 | 2336688 | 2335784 | − | tagatose 1,6-diphosphate aldolase |
| NMTN-08603 | SAV2192 | lacD | 2335708 | 2336688 | 2336300 | | |
| NMTN-09340 | SAV2193 | lacC | 2336692 | 2337624 | 2337089 | − | tagatose-6-phosphate kinase |
| NMTN-02796 | SAV2193 | lacC | 2336692 | 2337624 | 2337209 | | |
| NMTN-12681 | SAV2193 | lacC | 2336692 | 2337624 | 2337377 | | |
| NMTN-00103 | SAV2194 | lacB | 2337637 | 2338152 | 2337722 | − | galactose-6-phosphate isomerase LacB |
| | | | | | | | subunit |
| NMTN-03251 | SAV2194 | lacB | 2337637 | 2338152 | 2337894 | | |
| NMTN-11333 | SAV2197 | | 2339969 | 2340700 | 2340332 | + | hypothetical protein |
| NMTN-00445 | SAV2197 | | 2339969 | 2340700 | 2340405 | | |
| NMTN-10928 | SAV2197 | | 2339969 | 2340700 | 2340536 | | |
| NMTN-09119 | SAV2200 | | 2341421 | 2342269 | 2341570 | − | hypothetical protein |
| NMTN-15274 | SAV2201 | | 2342339 | 2342755 | 2342523 | − | hypothetical protein |
| NMTN-03517 | SAV2202 | hysA | 2343163 | 2345592 | 2343173 | + | hyaluronate lyase precursor |
| NMTN-07810 | SAV2202 | hysA | 2343163 | 2345592 | 2343213 | | |
| NMTN-09175 | SAV2202 | hysA | 2343163 | 2345592 | 2343330 | | |
| NMTN-12632 | SAV2202 | hysA | 2343163 | 2345592 | 2344019 | | |
| NMTN-12734 | SAV2202 | hysA | 2343163 | 2345592 | 2344495 | | |
| NMTN-07492 | SAV2202 | hysA | 2343163 | 2345592 | 2344531 | | |
| NMTN-05181 | SAV2202 | hysA | 2343163 | 2345592 | 2344645 | | |
| NMTN-09176 | SAV2202 | hysA | 2343163 | 2345592 | 2345140 | | |
| NMTN-12358 | SAV2202 | hysA | 2343163 | 2345592 | 2345249 | | |
| NMTN-09278 | SAV2202 | hysA | 2343163 | 2345592 | 2345553 | | |
| NMTN-08970 | SAV2203 | | 2345652 | 2346506 | 2345768 | − | conserved hypothetical protein |
| NMTN-12521 | SAV2203 | | 2345652 | 2346506 | 2345768 | | |
| NMTN-12117 | SAV2203 | | 2345652 | 2346506 | 2346414 | | |
| NMTN-04489 | SAV2204 | | 2346717 | 2347532 | 2347084 | − | conserved hypothetical protein |
| NMTN-02906 | SAV2204 | | 2346717 | 2347532 | 2347201 | | |
| NMTN-08826 | SAV2206 | | 2348636 | 2349340 | 2349110 | − | hypothetical protein |
| NMTN-11932 | SAV2207 | alsS | 2349377 | 2351041 | 2350115 | − | alpha-acetolactate synthase |
| NMTN-08611 | SAV2207 | alsS | 2349377 | 2351041 | 2350642 | | |
| NMTN-01630 | SAV2207 | alsS | 2349377 | 2351041 | 2350750 | | |
| NMTN-05445 | SAV2207 | alsS | 2349377 | 2351041 | 2350750 | | |
| NMTN-03959 | SAV2207 | alsS | 2349377 | 2351041 | 2350804 | | |
| NMTN-12811 | SAV2207 | alsS | 2349377 | 2351041 | 2350902 | | |
| NMTN-06362 | SAV2219 | truA | 2360488 | 2361291 | 2361035 | − | tRNA pseudouridine synthase A |
| NMTN-05576 | SAV2252 | | 2378567 | 2378956 | 2378726 | + | hypothetical protein |
| NMTN-06866 | SAV2252 | | 2378567 | 2378956 | 2378762 | | |
| NMTN-14302 | SAV2252 | | 2378567 | 2378956 | 2378871 | | |
| NMTN-06286 | SAV2252 | | 2378567 | 2378956 | 2378895 | | |
| NMTN-01570 | SAV2252 | | 2378567 | 2378956 | 2378935 | | |
| NMTN-12745 | SAV2253 | | 2379147 | 2380481 | 2379386 | − | conserved hypothetical protein |
| NMTN-00068 | SAV2253 | | 2379147 | 2380481 | 2379974 | | |
| NMTN-05204 | SAV2253 | | 2379147 | 2380481 | 2379979 | | |
| NMTN-05438 | SAV2253 | | 2379147 | 2380481 | 2380133 | | |
| NMTN-09409 | SAV2253 | | 2379147 | 2380481 | 2380191 | | |
| NMTN-05017 | SAV2254 | topB | 2380594 | 2382729 | 2381734 | − | DNA topoisomerase III topB |
| NMTN-13837 | SAV2254 | topB | 2380594 | 2382729 | 2381734 | | |
| NMTN-12573 | SAV2254 | topB | 2380594 | 2382729 | 2381768 | | |
| NMTN-11561d | SAV2254 | topB | 2380594 | 2382729 | 2381855 | | |
| NMTN-07127 | SAV2254 | topB | 2380594 | 2382729 | 2381870 | | |
| NMTN-04663 | SAV2254 | topB | 2380594 | 2382729 | 2381897 | | |
| NMTN-03690 | SAV2254 | topB | 2380594 | 2382729 | 2381900 | | |
| NMTN-12971 | SAV2254 | topB | 2380594 | 2382729 | 2382131 | | |
| NMTN-13163 | SAV2254 | topB | 2380594 | 2382729 | 2382131 | | |
| NMTN-13550 | SAV2254 | topB | 2380594 | 2382729 | 2382131 | | |
| NMTN-13552 | SAV2254 | topB | 2380594 | 2382729 | 2382131 | | |
| NMTN-05391 | SAV2254 | topB | 2380594 | 2382729 | 2382187 | | |
| NMTN-04643 | SAV2254 | topB | 2380594 | 2382729 | 2382262 | | |
| NMTN-12279 | SAV2254 | topB | 2380594 | 2382729 | 2382690 | | |
| NMTN-04767 | SAV2254 | topB | 2380594 | 2382729 | 2382717 | | |
| NMTN-11660 | SAV2255 | | 2382814 | 2383791 | 2383516 | + | conserved hypothetical protein |
| NMTN-03598 | SAV2255 | | 2382814 | 2383791 | 2383557 | | |
| NMTN-01500 | SAV2256 | | 2383972 | 2384835 | 2384236 | + | glucose uptake protein homolog |
| NMTN-00123 | SAV2256 | | 2383972 | 2384835 | 2384473 | | |
| NMTN-07706 | SAV2256 | | 2383972 | 2384835 | 2384810 | | |
| NMTN-02909 | SAV2258 | | 2385490 | 2386398 | 2385685 | − | conserved hypothetical protein |
| NMTN-02917 | SAV2258 | | 2385490 | 2386398 | 2385685 | | |
| NMTN-02745 | SAV2258 | | 2385490 | 2386398 | 2385690 | | |
| NMTN-06951 | SAV2258 | | 2385490 | 2386398 | 2385729 | | |
| NMTN-01901 | SAV2258 | | 2385490 | 2386398 | 2385755 | | |
| NMTN-10666 | SAV2258 | | 2385490 | 2386398 | 2385871 | | |
| NMTN-11869 | SAV2258 | | 2385490 | 2386398 | 2385898 | | |
| NMTN-07061 | SAV2258 | | 2385490 | 2386398 | 2386197 | | |
| NMTN-11883 | SAV2261 | | 2387441 | 2390608 | 2388535 | − | hypothetical protein |
| NMTN-00283 | SAV2261 | | 2387441 | 2390608 | 2388663 | | |
| NMTN-05054 | SAV2261 | | 2387441 | 2390608 | 2389165 | | |
| NMTN-07468 | SAV2261 | | 2387441 | 2390608 | 2389249 | | |
| NMTN-00436 | SAV2261 | | 2387441 | 2390608 | 2389336 | | |
| NMTN-03285 | SAV2261 | | 2387441 | 2390608 | 2389671 | | |
| NMTN-10966 | SAV2265 | | 2393548 | 2393988 | 2393633 | + | hypothetical protein |
| NMTN-13918 | SAV2265 | | 2393548 | 2393988 | 2393638 | | |
| NMTN-03874 | SAV2266 | | 2393981 | 2395192 | 2394434 | + | hypothetical protein |
| NMTN-04875 | SAV2266 | | 2393981 | 2395192 | 2394467 | | |
| NMTN-04394 | SAV2266 | | 2393981 | 2395192 | 2394682 | | |
| NMTN-13333 | SAV2266 | | 2393981 | 2395192 | 2394751 | | |
| NMTN-01597 | SAV2266 | | 2393981 | 2395192 | 2394789 | | |
| NMTN-06524 | SAV2266 | | 2393981 | 2395192 | 2394872 | | |
| NMTN-07695 | SAV2266 | | 2393981 | 2395192 | 2395000 | | |
| NMTN-13636 | SAV2268 | moaA | 2396221 | 2397243 | 2396489 | − | molybdenum cofactor biosynthesis |
| | | | | | | | protein A |
| NMTN-11964 | SAV2268 | moaA | 2396221 | 2397243 | 2396687 | | |
| NMTN-11592 | SAV2268 | moaA | 2396221 | 2397243 | 2396994 | | |
| NMTN-05250 | SAV2268 | moaA | 2396221 | 2397243 | 2397048 | | |
| NMTN-09182 | SAV2269 | mobA | 2397263 | 2397862 | 2397258 | − | molybdopterin-guanine dinucleotide |
| | | | | | | | biosynthesis |
| NMTN-10620 | SAV2269 | mobA | 2397263 | 2397862 | 2397409 | | |
| NMTN-04699 | SAV2269 | mobA | 2397263 | 2397862 | 2397757 | | |
| NMTN-03509 | SAV2270 | moaD | 2397869 | 2398102 | 2398066 | − | probable molybdopterin synthase small |
| | | | | | | | subunit |
| NMTN-01567 | SAV2271 | moaE | 2398108 | 2398554 | 2398353 | − | molybdopterin converting factor |
| NMTN-03209 | SAV2271 | moaE | 2398108 | 2398554 | 2398399 | | |
| NMTN-11375 | SAV2273 | moeA | 2399050 | 2400309 | 2399100 | − | molybdopterin biosynthesis protein |
| NMTN-12113 | SAV2273 | moeA | 2399050 | 2400309 | 2399208 | | |
| NMTN-13820 | SAV2273 | moeA | 2399050 | 2400309 | 2399211 | | |
| NMTN-14649 | SAV2273 | moeA | 2399050 | 2400309 | 2399927 | | |
| NMTN-00662 | SAV2273 | moeA | 2399050 | 2400309 | 2399930 | | |
| NMTN-12616 | SAV2274 | moaC | 2400372 | 2400866 | 2400519 | + | molybdenum cofactor biosynthesis |
| | | | | | | | protein C |
| NMTN-12987 | SAV2274 | moaC | 2400372 | 2400866 | 2400561 | | |
| NMTN-03257 | SAV2274 | moaC | 2400372 | 2400866 | 2400623 | | |
| NMTN-12467 | SAV2276 | moeB | 2401405 | 2402409 | 2401931 | − | molybdopterin biosynthesis protein |
| NMTN-04648 | SAV2277 | modC | 2402579 | 2403184 | 2403043 | − | molybdenum transport ATP-binding |
| | | | | | | | protein |
| NMTN-02985 | SAV2279 | | 2403826 | 2404038 | 2403961 | + | probable molybdate-binding protein |
| NMTN-04839 | SAV2280 | narQ | 2404380 | 2405177 | 2404457 | + | FdhD protein homolog |
| NMTN-05029 | SAV2280 | narQ | 2404380 | 2405177 | 2404457 | | |
| NMTN-09544 | SAV2280 | narQ | 2404380 | 2405177 | 2404579 | | |
| NMTN-07077 | SAV2280 | narQ | 2404380 | 2405177 | 2404648 | | |
| NMTN-03843 | SAV2281 | | 2405349 | 2406119 | 2406093 | − | hypothetical protein |
| NMTN-08709 | SAV2281 | | 2405349 | 2406119 | 2406093 | | |
| NMTN-09551 | SAV2281 | | 2405349 | 2406119 | 2406106 | | |
| NMTN-04375 | SAV2283 | | 2406871 | 2407812 | 2406924 | − | hypothetical protein |
| NMTN-03239 | SAV2283 | | 2406871 | 2407812 | 2407374 | | |
| NMTN-11554 | SAV2284 | | 2408210 | 2409118 | 2408265 | − | hypothetical protein |
| NMTN-06626 | SAV2284 | | 2408210 | 2409118 | 2408277 | | |
| NMTN-13637 | SAV2284 | | 2408210 | 2409118 | 2408509 | | |
| NMTN-03275 | SAV2285 | | 2409332 | 2410486 | 2410380 | − | hypothetical protein |
| NMTN-02966 | SAV2287 | | 2410703 | 2411608 | 2411059 | − | hypothetical protein |
| NMTN-14486 | SAV2287 | | 2410703 | 2411608 | 2411320 | | |
| NMTN-05057 | SAV2287 | | 2410703 | 2411608 | 2411530 | | |
| NMTN-03011 | SAV2287 | | 2410703 | 2411608 | 2411560 | | |
| NMTN-15250 | SAV2288 | ureA | 2411868 | 2412170 | 2412174 | + | urease gamma subunit |
| NMTN-15250 | SAV2289 | ureB | 2412184 | 2412594 | 2412174 | + | urease beta subunit |
| NMTN-07749d | SAV2289 | ureB | 2412184 | 2412594 | 2412351 | | |
| NMTN-05068 | SAV2290 | ureC | 2412591 | 2414306 | 2412913 | + | urease alpha subunit |
| NMTN-00837 | SAV2290 | ureC | 2412591 | 2414306 | 2413999 | | |
| NMTN-02942 | SAV2290 | ureC | 2412591 | 2414306 | 2414231 | | |
| NMTN-09184 | SAV2292 | ureF | 2414764 | 2415453 | 2415230 | + | urease accessory protein |
| NMTN-14054 | SAV2292 | ureF | 2414764 | 2415453 | 2415310 | | |
| NMTN-14482 | SAV2293 | ureG | 2415466 | 2416080 | 2415749 | + | urease accessory protein |
| NMTN-01475 | SAV2293 | ureG | 2415466 | 2416080 | 2415763 | | |
| NMTN-14648 | SAV2293 | ureG | 2415466 | 2416080 | 2415882 | | |
| NMTN-12725 | SAV2294 | ureD | 2416272 | 2416916 | 2416333 | + | urease accessory protein |
| NMTN-03045 | SAV2297 | | 2418270 | 2419013 | 2418646 | − | hypothetical protein |
| NMTN-11387 | SAV2297 | | 2418270 | 2419013 | 2418707 | | |
| NMTN-06774 | SAV2298 | | 2419039 | 2420994 | 2419175 | − | hypothetical protein |
| NMTN-10717 | SAV2298 | | 2419039 | 2420994 | 2419208 | | |
| NMTN-10804 | SAV2298 | | 2419039 | 2420994 | 2419323 | | |
| NMTN-03399 | SAV2298 | | 2419039 | 2420994 | 2419732 | | |
| NMTN-06159 | SAV2298 | | 2419039 | 2420994 | 2419854 | | |
| NMTN-13119 | SAV2298 | | 2419039 | 2420994 | 2420360 | | |
| NMTN-03776 | SAV2298 | | 2419039 | 2420994 | 2420432 | | |
| NMTN-02820 | SAV2298 | | 2419039 | 2420994 | 2420598 | | |
| NMTN-10716 | SAV2298 | | 2419039 | 2420994 | 2420715 | | |
| NMTN-13598 | SAV2298 | | 2419039 | 2420994 | 2420793 | | |
| NMTN-03952 | SAV2298 | | 2419039 | 2420994 | 2420900 | | |
| NMTN-03230 | SAV2298 | | 2419039 | 2420994 | 2420925 | | |
| NMTN-09996 | SAV2300 | | 2422425 | 2422553 | 2422450 | − | hypothetical protein |
| NMTN-09197 | SAV2301 | | 2422841 | 2424241 | 2422879 | − | hypothetical protein |
| NMTN-00354 | SAV2301 | | 2422841 | 2424241 | 2422915 | | |
| NMTN-06511 | SAV2301 | | 2422841 | 2424241 | 2423160 | | |
| NMTN-10267 | SAV2301 | | 2422841 | 2424241 | 2423186 | | |
| NMTN-10990 | SAV2301 | | 2422841 | 2424241 | 2423218 | | |
| NMTN-04678 | SAV2301 | | 2422841 | 2424241 | 2423359 | | |
| NMTN-05291 | SAV2301 | | 2422841 | 2424241 | 2423533 | | |
| NMTN-03158 | SAV2301 | | 2422841 | 2424241 | 2423688 | | |
| NMTN-11699 | SAV2301 | | 2422841 | 2424241 | 2423695 | | |
| NMTN-02916 | SAV2301 | | 2422841 | 2424241 | 2423778 | | |
| NMTN-11716 | SAV2302 | | 2424336 | 2425418 | 2424922 | − | hypothetical protein |
| NMTN-05455 | SAV2304 | | 2426325 | 2426825 | 2426514 | + | hypothetical protein |
| NMTN-09630 | SAV2305 | | 2427186 | 2428139 | 2427190 | + | hypothetical protein |
| NMTN-11098 | SAV2305 | | 2427186 | 2428139 | 2427921 | | |
| NMTN-09992 | SAV2306 | | 2428231 | 2429355 | 2428303 | − | hypothetical protein |
| NMTN-12425 | SAV2306 | | 2428231 | 2429355 | 2428416 | | |
| NMTN-11352 | SAV2306 | | 2428231 | 2429355 | 2428746 | | |
| NMTN-07910 | SAV2306 | | 2428231 | 2429355 | 2428802 | | |
| NMTN-05523 | SAV2306 | | 2428231 | 2429355 | 2428863 | | |
| NMTN-03759 | SAV2306 | | 2428231 | 2429355 | 2428929 | | |
| NMTN-09916 | SAV2306 | | 2428231 | 2429355 | 2428994 | | |
| NMTN-06775 | SAV2306 | | 2428231 | 2429355 | 2429112 | | |
| NMTN-07399 | SAV2306 | | 2428231 | 2429355 | 2429112 | | |
| NMTN-11146 | SAV2306 | | 2428231 | 2429355 | 2429195 | | |
| NMTN-09134 | SAV2307 | | 2429666 | 2430442 | 2430373 | − | hypothetical protein |
| NMTN-14520 | SAV2308 | | 2430925 | 2431398 | 2431160 | − | conserved hypothetical protein |
| NMTN-07728 | SAV2309 | | 2431398 | 2434352 | 2431625 | − | formate dehydrogenase homolog |
| NMTN-02123 | SAV2309 | | 2431398 | 2434352 | 2431976 | | |
| NMTN-12601 | SAV2309 | | 2431398 | 2434352 | 2432603 | | |
| NMTN-11680 | SAV2309 | | 2431398 | 2434352 | 2432743 | | |
| NMTN-04836 | SAV2309 | | 2431398 | 2434352 | 2432992 | | |
| NMTN-07161 | SAV2309 | | 2431398 | 2434352 | 2433079 | | |
| NMTN-06344 | SAV2309 | | 2431398 | 2434352 | 2433168 | | |
| NMTN-07944 | SAV2309 | | 2431398 | 2434352 | 2433232 | | |
| NMTN-07435 | SAV2309 | | 2431398 | 2434352 | 2433353 | | |
| NMTN-07293 | SAV2309 | | 2431398 | 2434352 | 2433406 | | |
| NMTN-11081 | SAV2309 | | 2431398 | 2434352 | 2434068 | | |
| NMTN-03707 | SAV2310 | | 2434955 | 2435902 | 2435772 | − | hypothetical protein |
| NMTN-13576 | SAV2311 | | 2436035 | 2436832 | 2436038 | − | hypothetical protein |
| NMTN-05647 | SAV2311 | | 2436035 | 2436832 | 2436475 | | |
| NMTN-05648 | SAV2311 | | 2436035 | 2436832 | 2436475 | | |
| NMTN-13415 | SAV2311 | | 2436035 | 2436832 | 2436475 | | |
| NMTN-03164 | SAV2311 | | 2436035 | 2436832 | 2436524 | | |
| NMTN-11659 | SAV2311 | | 2436035 | 2436832 | 2436697 | | |
| NMTN-10183 | SAV2313 | | 2437934 | 2438671 | 2437950 | − | hypothetical protein |
| NMTN-11440 | SAV2313 | | 2437934 | 2438671 | 2438139 | | |
| NMTN-06248 | SAV2313 | | 2437934 | 2438671 | 2438180 | | |
| NMTN-06318 | SAV2313 | | 2437934 | 2438671 | 2438193 | | |
| NMTN-11367 | SAV2313 | | 2437934 | 2438671 | 2438217 | | |
| NMTN-12135 | SAV2313 | | 2437934 | 2438671 | 2438326 | | |
| NMTN-00848 | SAV2313 | | 2437934 | 2438671 | 2438345 | | |
| NMTN-12984 | SAV2313 | | 2437934 | 2438671 | 2438367 | | |
| NMTN-12172 | SAV2313 | | 2437934 | 2438671 | 2438462 | | |
| NMTN-09178 | SAV2313 | | 2437934 | 2438671 | 2438561 | | |
| NMTN-04299 | SAV2313 | | 2437934 | 2438671 | 2438584 | | |
| NMTN-03658 | SAV2313 | | 2437934 | 2438671 | 2438604 | | |
| NMTN-09064 | SAV2313 | | 2437934 | 2438671 | 2438634 | | |
| NMTN-12825 | SAV2313 | | 2437934 | 2438671 | 2438635 | | |
| NMTN-02969 | SAV2314 | | 2438720 | 2439049 | 2438895 | + | hypothetical protein |
| NMTN-05372 | SAV2315 | | 2439494 | 2440366 | 2439518 | + | hypothetical protein |
| NMTN-10942 | SAV2315 | | 2439494 | 2440366 | 2440002 | | |
| NMTN-02289 | SAV2315 | | 2439494 | 2440366 | 2440086 | | |
| NMTN-13744 | SAV2316 | | 2440753 | 2442132 | 2440887 | − | hypothetical protein |
| NMTN-13793 | SAV2316 | | 2440753 | 2442132 | 2440887 | | |
| NMTN-13797 | SAV2316 | | 2440753 | 2442132 | 2440887 | | |
| NMTN-13810 | SAV2316 | | 2440753 | 2442132 | 2440887 | | |
| NMTN-13823 | SAV2316 | | 2440753 | 2442132 | 2440887 | | |
| NMTN-13836 | SAV2316 | | 2440753 | 2442132 | 2440887 | | |
| NMTN-13842 | SAV2316 | | 2440753 | 2442132 | 2440887 | | |
| NMTN-13844 | SAV2316 | | 2440753 | 2442132 | 2440887 | | |
| NMTN-13848 | SAV2316 | | 2440753 | 2442132 | 2440887 | | |
| NMTN-13855 | SAV2316 | | 2440753 | 2442132 | 2440887 | | |
| NMTN-13845 | SAV2316 | | 2440753 | 2442132 | 2440888 | | |
| NMTN-13851 | SAV2316 | | 2440753 | 2442132 | 2440890 | | |
| NMTN-11847 | SAV2316 | | 2440753 | 2442132 | 2440893 | | |
| NMTN-04443 | SAV2316 | | 2440753 | 2442132 | 2440903 | | |
| NMTN-14572 | SAV2316 | | 2440753 | 2442132 | 2441240 | | |
| NMTN-01488 | SAV2316 | | 2440753 | 2442132 | 2441265 | | |
| NMTN-13109 | SAV2316 | | 2440753 | 2442132 | 2441652 | | |
| NMTN-11066 | SAV2316 | | 2440753 | 2442132 | 2441654 | | |
| NMTN-14409 | SAV2317 | | 2442412 | 2442768 | 2442511 | + | hypothetical protein |
| NMTN-10856 | SAV2317 | | 2442412 | 2442768 | 2442567 | | |
| NMTN-10918 | SAV2318 | | 2442765 | 2442950 | 2442909 | + | hypothetical protein |
| NMTN-04077 | SAV2318 | | 2442765 | 2442950 | 2442941 | | |
| NMTN-12860d | SAV2320 | | 2443248 | 2443883 | 2443819 | − | hypothetical protein |
| NMTN-13016 | SAV2321 | | 2443958 | 2444875 | 2443949 | − | hypothetical protein |
| NMTN-09522 | SAV2321 | | 2443958 | 2444875 | 2443999 | | |
| NMTN-03233 | SAV2321 | | 2443958 | 2444875 | 2444325 | | |
| NMTN-12634 | SAV2321 | | 2443958 | 2444875 | 2444400 | | |
| NMTN-09158 | SAV2321 | | 2443958 | 2444875 | 2444533 | | |
| NMTN-12342 | SAV2322 | | 2445143 | 2445673 | 2445649 | + | hypothetical protein |
| NMTN-12084 | SAV2323 | glvC | 2445739 | 2447343 | 2445809 | − | PTS system, arbutin-like IIBC |
| | | | | | | | component |
| NMTN-08461 | SAV2323 | glvC | 2445739 | 2447343 | 2445986 | | |
| NMTN-13046 | SAV2323 | glvC | 2445739 | 2447343 | 2446457 | | |
| NMTN-09671 | SAV2323 | glvC | 2445739 | 2447343 | 2446548 | | |
| NMTN-08862 | SAV2323 | glvC | 2445739 | 2447343 | 2446642 | | |
| NMTN-11950 | SAV2323 | glvC | 2445739 | 2447343 | 2446880 | | |
| NMTN-13912 | SAV2323 | glvC | 2445739 | 2447343 | 2446882 | | |
| NMTN-09308 | SAV2323 | glvC | 2445739 | 2447343 | 2447013 | | |
| NMTN-12551 | SAV2324 | | 2447673 | 2448437 | 2448305 | + | hypothetical protein |
| NMTN-12788 | SAV2324 | | 2447673 | 2448437 | 2448373 | | |
| NMTN-05514 | SAV2326 | | 2449168 | 2450547 | 2449256 | − | hypothetical protein |
| NMTN-12089 | SAV2326 | | 2449168 | 2450547 | 2449367 | | |
| NMTN-05520 | SAV2326 | | 2449168 | 2450547 | 2449637 | | |
| NMTN-11827 | SAV2326 | | 2449168 | 2450547 | 2449637 | | |
| NMTN-11688 | SAV2326 | | 2449168 | 2450547 | 2449813 | | |
| NMTN-06256 | SAV2326 | | 2449168 | 2450547 | 2449902 | | |
| NMTN-06359 | SAV2326 | | 2449168 | 2450547 | 2449905 | | |
| NMTN-03267 | SAV2326 | | 2449168 | 2450547 | 2449916 | | |
| NMTN-00431 | SAV2326 | | 2449168 | 2450547 | 2450108 | | |
| NMTN-00733 | SAV2326 | | 2449168 | 2450547 | 2450109 | | |
| NMTN-13882 | SAV2326 | | 2449168 | 2450547 | 2450472 | | |
| NMTN-07553 | SAV2326 | | 2449168 | 2450547 | 2450522 | | |
| NMTN-07378 | SAV2327 | | 2450706 | 2451275 | 2450975 | − | hypothetical protein |
| NMTN-04511 | SAV2327 | | 2450706 | 2451275 | 2451020 | | |
| NMTN-00675 | SAV2327 | | 2450706 | 2451275 | 2451087 | | |
| NMTN-04482 | SAV2327 | | 2450706 | 2451275 | 2451183 | | |
| NMTN-08886 | SAV2328 | | 2451762 | 2452643 | 2452435 | − | hypothetical protein |
| NMTN-14623 | SAV2329 | | 2452754 | 2453875 | 2452829 | − | hypothetical protein |
| NMTN-02709 | SAV2329 | | 2452754 | 2453875 | 2453259 | | |
| NMTN-01836 | SAV2329 | | 2452754 | 2453875 | 2453367 | | |
| NMTN-08148 | SAV2329 | | 2452754 | 2453875 | 2453383 | | |
| NMTN-02283 | SAV2330 | hutI | 2454151 | 2455389 | 2454294 | − | imidazolonepropionase |
| NMTN-11618d | SAV2330 | hutI | 2454151 | 2455389 | 2454543 | | |
| NMTN-11803 | SAV2330 | hutI | 2454151 | 2455389 | 2454543 | | |
| NMTN-00804 | SAV2330 | hutI | 2454151 | 2455389 | 2454731 | | |
| NMTN-04251 | SAV2330 | hutI | 2454151 | 2455389 | 2454866 | | |
| NMTN-11400 | SAV2330 | hutI | 2454151 | 2455389 | 2454990 | | |
| NMTN-07634 | SAV2331 | hutU | 2455389 | 2457050 | 2455768 | − | urocanate hydratase |
| NMTN-03839 | SAV2331 | hutU | 2455389 | 2457050 | 2456688 | | |
| NMTN-11095 | SAV2331 | hutU | 2455389 | 2457050 | 2456871 | | |
| NMTN-11894 | SAV2332 | | 2457210 | 2458094 | 2457283 | + | hypothetical protein |
| NMTN-12577 | SAV2332 | | 2457210 | 2458094 | 2457434 | | |
| NMTN-02817 | SAV2332 | | 2457210 | 2458094 | 2457602 | | |
| NMTN-11235 | SAV2332 | | 2457210 | 2458094 | 2457907 | | |
| NMTN-09848 | SAV2333(?) | fosB | 2458336 | 2458755 | 2458416 | | |
| NMTN-06569 | SAV2333 | fosB | 2458336 | 2458755 | 2458563 | + | fosfomycin resistance protein fofB |
| NMTN-09451 | SAV2333 | fosB | 2458336 | 2458755 | 2458634 | | |
| NMTN-03723 | SAV2334 | | 2459011 | 2459946 | 2459822 | − | hypothetical protein |
| NMTN-12843 | SAV2335 | | 2460236 | 2461495 | 2460360 | + | hypothetical protein |
| NMTN-09166 | SAV2335 | | 2460236 | 2461495 | 2461142 | | |
| NMTN-12541 | SAV2336 | | 2461561 | 2462247 | 2461679 | − | hypothetical protein |
| NMTN-02438 | SAV2336 | | 2461561 | 2462247 | 2462037 | | |
| NMTN-01722 | SAV2337 | | 2462421 | 2463074 | 2462427 | + | conserved hypothetical protein |
| NMTN-04698 | SAV2337 | | 2462421 | 2463074 | 2462617 | | |
| NMTN-08290 | SAV2337 | | 2462421 | 2463074 | 2462828 | | |
| NMTN-05245 | SAV2337 | | 2462421 | 2463074 | 2462833 | | |
| NMTN-08112 | SAV2337 | | 2462421 | 2463074 | 2462867 | | |
| NMTN-11425 | SAV2337 | | 2462421 | 2463074 | 2462967 | | |
| NMTN-00614 | SAV2337 | | 2462421 | 2463074 | 2463071 | | |
| NMTN-10459 | SAV2338 | | 2463310 | 2464329 | 2463338 | − | hypothetical protein |
| NMTN-08910 | SAV2338 | | 2463310 | 2464329 | 2463572 | | |
| NMTN-00669 | SAV2338 | | 2463310 | 2464329 | 2463590 | | |
| NMTN-07753 | SAV2338 | | 2463310 | 2464329 | 2463730 | | |
| NMTN-08527 | SAV2338 | | 2463310 | 2464329 | 2463735 | | |
| NMTN-11766 | SAV2338 | | 2463310 | 2464329 | 2463745 | | |
| NMTN-09180 | SAV2338 | | 2463310 | 2464329 | 2463753 | | |
| NMTN-04091 | SAV2338 | | 2463310 | 2464329 | 2463898 | | |
| NMTN-14700 | SAV2338 | | 2463310 | 2464329 | 2464208 | | |
| NMTN-11404 | SAV2339 | | 2464359 | 2464685 | 2464660 | − | conserved hypothetical protein |
| NMTN-03272 | SAV2340 | | 2464903 | 2466135 | 2464970 | − | conserved hypothetical protein |
| NMTN-02355 | SAV2340 | | 2464903 | 2466135 | 2465260 | | |
| NMTN-11015 | SAV2340 | | 2464903 | 2466135 | 2465915 | | |
| NMTN-09204 | SAV2340 | | 2464903 | 2466135 | 2465950 | | |
| NMTN-13403 | SAV2340 | | 2464903 | 2466135 | 2465953 | | |
| NMTN-04934 | SAV2340 | | 2464903 | 2466135 | 2466113 | | |
| NMTN-06075 | SAV2341 | | 2466128 | 2467027 | 2466160 | − | hypothetical protein |
| NMTN-12933 | SAV2341 | | 2466128 | 2467027 | 2466568 | | |
| NMTN-11486d | SAV2342 | | 2467258 | 2467878 | 2467407 | − | conserved hypothetical protein |
| NMTN-08332 | SAV2342 | | 2467258 | 2467878 | 2467484 | | |
| NMTN-01529 | SAV2342 | | 2467258 | 2467878 | 2467563 | | |
| NMTN-09226 | SAV2342 | | 2467258 | 2467878 | 2467860 | | |
| NMTN-07869 | SAV2344 | | 2468277 | 2468885 | 2468325 | + | hypothetical protein |
| NMTN-12094 | SAV2344 | | 2468277 | 2468885 | 2468325 | | |
| NMTN-12247 | SAV2344 | | 2468277 | 2468885 | 2468363 | | |
| NMTN-03262 | SAV2344 | | 2468277 | 2468885 | 2468728 | | |
| NMTN-00352 | SAV2345 | | 2469218 | 2470426 | 2469835 | + | hypothetical protein |
| NMTN-01587 | SAV2347 | | 2471554 | 2472501 | 2471718 | − | hypothetical protein |
| NMTN-03137 | SAV2347 | | 2471554 | 2472501 | 2471883 | | |
| NMTN-12748 | SAV2347 | | 2471554 | 2472501 | 2471883 | | |
| NMTN-13356 | SAV2347 | | 2471554 | 2472501 | 2471970 | | |
| NMTN-00098 | SAV2347 | | 2471554 | 2472501 | 2471975 | | |
| NMTN-07253 | SAV2347 | | 2471554 | 2472501 | 2472041 | | |
| NMTN-00096 | SAV2347 | | 2471554 | 2472501 | 2472145 | | |
| NMTN-07667 | SAV2347 | | 2471554 | 2472501 | 2472184 | | |
| NMTN-06759 | SAV2348 | | 2472604 | 2473089 | 2472671 | + | conserved hypothetical protein |
| NMTN-08427 | SAV2348 | | 2472604 | 2473089 | 2473059 | | |
| NMTN-01008 | SAV2349 | | 2473086 | 2473505 | 2473238 | + | hypothetical protein |
| NMTN-11582 | SAV2350 | | 2473613 | 2474533 | 2473627 | − | hypothetical protein |
| NMTN-13287 | SAV2350 | | 2473613 | 2474533 | 2473708 | | |
| NMTN-06350 | SAV2350 | | 2473613 | 2474533 | 2473736 | | |
| NMTN-09183 | SAV2350 | | 2473613 | 2474533 | 2474125 | | |
| NMTN-11256 | SAV2350 | | 2473613 | 2474533 | 2474212 | | |
| NMTN-03654 | SAV2352 | | 2475625 | 2477556 | 2476084 | − | hypothetical protein |
| NMTN-05224 | SAV2352 | | 2475625 | 2477556 | 2476084 | | |
| NMTN-03560 | SAV2352 | | 2475625 | 2477556 | 2477360 | | |
| NMTN-05190 | SAV2354 | | 2478337 | 2478891 | 2478737 | + | hypothetical protein |
| NMTN-02808 | SAV2354 | | 2478337 | 2478891 | 2478772 | | |
| NMTN-05284 | SAV2354 | | 2478337 | 2478891 | 2478772 | | |
| NMTN-02193 | SAV2355 | tcaB | 2479118 | 2480326 | 2479305 | − | TcaB protein |
| NMTN-12474 | SAV2355 | tcaB | 2479118 | 2480326 | 2479524 | | |
| NMTN-06220 | SAV2355 | tcaB | 2479118 | 2480326 | 2480173 | | |
| NMTN-08156 | SAV2355 | tcaB | 2479118 | 2480326 | 2480213 | | |
| NMTN-07025 | SAV2356 | tcaA | 2480595 | 2481977 | 2480671 | − | TcaA protein |
| NMTN-00737 | SAV2356 | tcaA | 2480595 | 2481977 | 2480986 | | |
| NMTN-07469 | SAV2356 | tcaA | 2480595 | 2481977 | 2481077 | | |
| NMTN-11450 | SAV2356 | tcaA | 2480595 | 2481977 | 2481199 | | |
| NMTN-06280 | SAV2356 | tcaA | 2480595 | 2481977 | 2481627 | | |
| NMTN-01835 | SAV2356 | tcaA | 2480595 | 2481977 | 2481638 | | |
| NMTN-05293 | SAV2358 | | 2482867 | 2483835 | 2482884 | + | hypothetical protein |
| NMTN-05203 | SAV2358 | | 2482867 | 2483835 | 2483171 | | |
| NMTN-12350 | SAV2358 | | 2482867 | 2483835 | 2483242 | | |
| NMTN-08005 | SAV2358 | | 2482867 | 2483835 | 2483352 | | |
| NMTN-09058 | SAV2358 | | 2482867 | 2483835 | 2483539 | | |
| NMTN-06457 | SAV2358 | | 2482867 | 2483835 | 2483749 | | |
| NMTN-03177 | SAV2359 | | 2484066 | 2484731 | 2484218 | − | hypothetical protein |
| NMTN-05158 | SAV2360 | | 2484731 | 2485786 | 2484814 | − | conserved hypothetical protein |
| NMTN-11356 | SAV2360 | | 2484731 | 2485786 | 2484814 | | |
| NMTN-13048 | SAV2360 | | 2484731 | 2485786 | 2484816 | | |
| NMTN-04342 | SAV2360 | | 2484731 | 2485786 | 2484885 | | |
| NMTN-02054 | SAV2360 | | 2484731 | 2485786 | 2485134 | | |
| NMTN-01762 | SAV2360 | | 2484731 | 2485786 | 2485338 | | |
| NMTN-05560 | SAV2360 | | 2484731 | 2485786 | 2485444 | | |
| NMTN-02861 | SAV2360 | | 2484731 | 2485786 | 2485659 | | |
| NMTN-08752 | SAV2360 | | 2484731 | 2485786 | 2485738 | | |
| NMTN-02823 | SAV2361 | | 2485922 | 2486596 | 2486008 | + | hypothetical protein |
| NMTN-07744 | SAV2362 | | 2486589 | 2487962 | 2486748 | + | hypothetical protein |
| NMTN-01562 | SAV2362 | | 2486589 | 2487962 | 2486878 | | |
| NMTN-12100 | SAV2362 | | 2486589 | 2487962 | 2487601 | | |
| NMTN-08907 | SAV2363 | | 2488125 | 2488568 | 2488252 | + | conserved hypothetical protein |
| NMTN-11074 | SAV2365 | | 2489086 | 2490621 | 2489364 | − | hypothetical protein |
| NMTN-12238 | SAV2365 | | 2489086 | 2490621 | 2489487 | | |
| NMTN-08335 | SAV2365 | | 2489086 | 2490621 | 2490175 | | |
| NMTN-02616 | SAV2365 | | 2489086 | 2490621 | 2490255 | | |
| NMTN-11336 | SAV2365 | | 2489086 | 2490621 | 2490255 | | |
| NMTN-00141 | SAV2365 | | 2489086 | 2490621 | 2490320 | | |
| NMTN-06225 | SAV2365 | | 2489086 | 2490621 | 2490485 | | |
| NMTN-09784 | SAV2366 | | 2490945 | 2492543 | 2490971 | − | L-lactate permease lctP homolog |
| NMTN-12184 | SAV2366 | | 2490945 | 2492543 | 2491447 | | |
| NMTN-13172 | SAV2366 | | 2490945 | 2492543 | 2491577 | | |
| NMTN-12729 | SAV2366 | | 2490945 | 2492543 | 2491708 | | |
| NMTN-03728 | SAV2366 | | 2490945 | 2492543 | 2491897 | | |
| NMTN-12585 | SAV2366 | | 2490945 | 2492543 | 2492320 | | |
| NMTN-12244 | SAV2367 | | 2492860 | 2494533 | 2493262 | + | hypothetical protein |
| NMTN-09006 | SAV2367 | | 2492860 | 2494533 | 2493350 | | |
| NMTN-00287 | SAV2367 | | 2492860 | 2494533 | 2493583 | | |
| NMTN-07919 | SAV2367 | | 2492860 | 2494533 | 2493784 | | |
| NMTN-14020 | SAV2367 | | 2492860 | 2494533 | 2494081 | | |
| NMTN-08909 | SAV2367 | | 2492860 | 2494533 | 2494502 | | |
| NMTN-12925 | SAV2367 | | 2492860 | 2494533 | 2494505 | | |
| NMTN-04661 | SAV2367 | | 2492860 | 2494533 | 2494526 | | |
| NMTN-13460 | SAV2368 | | 2494687 | 2495301 | 2494679 | − | hypothetical protein |
| NMTN-02684 | SAV2368 | | 2494687 | 2495301 | 2494830 | | |
| NMTN-09868 | SAV2369 | | 2495615 | 2496130 | 2495678 | − | hypothetical protein |
| NMTN-07712 | SAV2369 | | 2495615 | 2496130 | 2495996 | | |
| NMTN-14755 | SAV2370 | | 2496321 | 2497325 | 2496603 | + | conserved hypothetical protein |
| NMTN-08714 | SAV2370 | | 2496321 | 2497325 | 2497019 | | |
| NMTN-03958 | SAV2370 | | 2496321 | 2497325 | 2497078 | | |
| NMTN-12606 | SAV2370 | | 2496321 | 2497325 | 2497081 | | |
| NMTN-08444 | SAV2370 | | 2496321 | 2497325 | 2497097 | | |
| NMTN-00825 | SAV2371 | | 2497371 | 2497772 | 2497383 | − | hypothetical protein |
| NMTN-04025 | SAV2371 | | 2497371 | 2497772 | 2497383 | | |
| NMTN-12872 | SAV2372 | | 2497808 | 2498803 | 2497813 | − | hypothetical protein |
| NMTN-05076 | SAV2372 | | 2497808 | 2498803 | 2498003 | | |
| NMTN-09222 | SAV2372 | | 2497808 | 2498803 | 2498191 | | |
| NMTN-03494 | SAV2372 | | 2497808 | 2498803 | 2498717 | | |
| NMTN-03496 | SAV2372 | | 2497808 | 2498803 | 2498717 | | |
| NMTN-02339 | SAV2373 | | 2499074 | 2499505 | 2499213 | − | hypothetical protein |
| NMTN-06415 | SAV2373 | | 2499074 | 2499505 | 2499271 | | |
| NMTN-00035 | SAV2373 | | 2499074 | 2499505 | 2499277 | | |
| NMTN-00682 | SAV2373 | | 2499074 | 2499505 | 2499378 | | |
| NMTN-06604 | SAV2374 | | 2499712 | 2500989 | 2499956 | − | hypothetical protein |
| NMTN-01600 | SAV2374 | | 2499712 | 2500989 | 2500220 | | |
| NMTN-02788 | SAV2374 | | 2499712 | 2500989 | 2500748 | | |
| NMTN-13740 | SAV2374 | | 2499712 | 2500989 | 2500799 | | |
| NMTN-07192 | SAV2375 | | 2501150 | 2501773 | 2501172 | + | hypothetical protein |
| NMTN-12259 | SAV2376 | | 2501819 | 2502763 | 2501859 | + | hypothetical protein |
| NMTN-04551 | SAV2376 | | 2501819 | 2502763 | 2502318 | | |
| NMTN-11435 | SAV2376 | | 2501819 | 2502763 | 2502524 | | |
| NMTN-06379 | SAV2376 | | 2501819 | 2502763 | 2502672 | | |
| NMTN-09345 | SAV2376 | | 2501819 | 2502763 | 2502674 | | |
| NMTN-02978 | SAV2376 | | 2501819 | 2502763 | 2502675 | | |
| NMTN-11695 | SAV2376 | | 2501819 | 2502763 | 2502676 | | |
| NMTN-10973 | SAV2376 | | 2501819 | 2502763 | 2502681 | | |
| NMTN-09938 | SAV2377 | scrA | 2502848 | 2504290 | 2502877 | − | PTS system, sucrose-specific IIBC |
| | | | | | | | component |
| NMTN-04741 | SAV2377 | scrA | 2502848 | 2504290 | 2503326 | | |
| NMTN-10649 | SAV2377 | scrA | 2502848 | 2504290 | 2503444 | | |
| NMTN-01749 | SAV2377 | scrA | 2502848 | 2504290 | 2503702 | | |
| NMTN-10433 | SAV2377 | scrA | 2502848 | 2504290 | 2504107 | | |
| NMTN-08917 | SAV2378 | | 2504335 | 2504811 | 2504441 | + | probable type II DNA modification |
| | | | | | | | enzyme |
| NMTN-12432 | SAV2381 | | 2507864 | 2509969 | 2508087 | + | hypothetical protein |
| NMTN-10953 | SAV2381 | | 2507864 | 2509969 | 2508156 | | |
| NMTN-09727 | SAV2381 | | 2507864 | 2509969 | 2508198 | | |
| NMTN-14440 | SAV2381 | | 2507864 | 2509969 | 2508228 | | |
| NMTN-13437 | SAV2381 | | 2507864 | 2509969 | 2508591 | | |
| NMTN-12704 | SAV2381 | | 2507864 | 2509969 | 2508734 | | |
| NMTN-11240 | SAV2381 | | 2507864 | 2509969 | 2508923 | | |
| NMTN-10838 | SAV2381 | | 2507864 | 2509969 | 2508959 | | |
| NMTN-05394 | SAV2381 | | 2507864 | 2509969 | 2509061 | | |
| NMTN-11858 | SAV2381 | | 2507864 | 2509969 | 2509246 | | |
| NMTN-03472 | SAV2381 | | 2507864 | 2509969 | 2509389 | | |
| NMTN-05469 | SAV2381 | | 2507864 | 2509969 | 2509454 | | |
| NMTN-07405 | SAV2381 | | 2507864 | 2509969 | 2509681 | | |
| NMTN-08874 | SAV2381 | | 2507864 | 2509969 | 2509695 | | |
| NMTN-14416 | SAV2382 | | 2510040 | 2510462 | 2510172 | | |
| NMTN-09162 | SAV2382 | | 2510040 | 2510462 | 2510249 | + | hypothetical protein |
| NMTN-03890 | SAV2383 | | 2510726 | 2511082 | 2510783 | + | hypothetical protein |
| NMTN-12863t | SAV2383 | | 2510726 | 2511082 | 2510934 | | |
| NMTN-09969 | SAV2384 | gltT | 2511400 | 2512677 | 2511443 | − | proton/sodium-glutamate symport |
| | | | | | | | protein |
| NMTN-14044 | SAV2384 | gltT | 2511400 | 2512677 | 2511516 | | |
| NMTN-13401 | SAV2384 | gltT | 2511400 | 2512677 | 2512507 | | |
| NMTN-14198 | SAV2384 | gltT | 2511400 | 2512677 | 2512529 | | |
| NMTN-00393 | SAV2385 | | 2512859 | 2513224 | 2513019 | − | hypothetical protein |
| NMTN-11825 | SAV2385 | | 2512859 | 2513224 | 2513161 | | |
| NMTN-00203 | SAV2385 | | 2512859 | 2513224 | 2513207 | | |
| NMTN-02310 | SAV2386 | | 2513621 | 2514067 | 2513626 | + | hypothetical protein |
| NMTN-13735 | SAV2386 | | 2513621 | 2514067 | 2513646 | | |
| NMTN-11343 | SAV2388 | narK | 2514818 | 2515987 | 2515129 | − | nitrite extrusion protein |
| NMTN-09494 | SAV2389 | | 2516356 | 2517270 | 2516486 | + | conserved hypothetical protein |
| NMTN-01640 | SAV2389 | | 2516356 | 2517270 | 2516732 | | |
| NMTN-04935 | SAV2389 | | 2516356 | 2517270 | 2516740 | | |
| NMTN-08759 | SAV2389 | | 2516356 | 2517270 | 2516770 | | |
| NMTN-00175 | SAV2389 | | 2516356 | 2517270 | 2516807 | | |
| NMTN-04739 | SAV2389 | | 2516356 | 2517270 | 2516851 | | |
| NMTN-10379 | SAV2389 | | 2516356 | 2517270 | 2517175 | | |
| NMTN-05073 | SAV2389 | | 2516356 | 2517270 | 2517183 | | |
| NMTN-13220 | SAV2389 | | 2516356 | 2517270 | 2517254 | | |
| NMTN-13220 | SAV2390 | | 2517249 | 2517989 | 2517254 | + | hypothetical protein |
| NMTN-07053 | SAV2390 | | 2517249 | 2517989 | 2517840 | | |
| NMTN-11392 | SAV2392 | | 2518923 | 2519957 | 2519129 | − | hypothetical protein |
| NMTN-12130 | SAV2392 | | 2518923 | 2519957 | 2519236 | | |
| NMTN-10861 | SAV2392 | | 2518923 | 2519957 | 2519351 | | |
| NMTN-09170 | SAV2392 | | 2518923 | 2519957 | 2519549 | | |
| NMTN-11284 | SAV2392 | | 2518923 | 2519957 | 2519683 | | |
| NMTN-00428 | SAV2392 | | 2518923 | 2519957 | 2519723 | | |
| NMTN-03252 | SAV2392 | | 2518923 | 2519957 | 2519926 | | |
| NMTN-10970 | SAV2393 | | 2519981 | 2520433 | 2520330 | − | hypothetical protein |
| NMTN-03286 | SAV2394 | narI | 2520453 | 2521130 | 2520569 | − | nitrate reductase gamma chain |
| NMTN-11610 | SAV2394 | narI | 2520453 | 2521130 | 2520688 | | |
| NMTN-05446 | SAV2394 | narI | 2520453 | 2521130 | 2520839 | | |
| NMTN-14635 | SAV2394 | narI | 2520453 | 2521130 | 2520841 | | |
| NMTN-00117 | SAV2394 | narI | 2520453 | 2521130 | 2520872 | | |
| NMTN-10203 | SAV2394 | narI | 2520453 | 2521130 | 2521126 | | |
| NMTN-11370 | SAV2395 | | 2521123 | 2521698 | 2521238 | − | hypothetical protein |
| NMTN-11516 | SAV2395 | | 2521123 | 2521698 | 2521240 | | |
| NMTN-03599 | SAV2395 | | 2521123 | 2521698 | 2521462 | | |
| NMTN-12302 | SAV2395 | | 2521123 | 2521698 | 2521462 | | |
| NMTN-12245 | SAV2396 | narH | 2521691 | 2523250 | 2521824 | − | nitrate reductase beta chain |
| NMTN-11725 | SAV2396 | narH | 2521691 | 2523250 | 2521916 | | |
| NMTN-10950 | SAV2396 | narH | 2521691 | 2523250 | 2522077 | | |
| NMTN-11915 | SAV2396 | narH | 2521691 | 2523250 | 2522166 | | |
| NMTN-13856 | SAV2396 | narH | 2521691 | 2523250 | 2522297 | | |
| NMTN-07613 | SAV2396 | narH | 2521691 | 2523250 | 2522325 | | |
| NMTN-13049 | SAV2396 | narH | 2521691 | 2523250 | 2522358 | | |
| NMTN-06688 | SAV2396 | narH | 2521691 | 2523250 | 2522362 | | |
| NMTN-12779 | SAV2396 | narH | 2521691 | 2523250 | 2522431 | | |
| NMTN-00827 | SAV2396 | narH | 2521691 | 2523250 | 2522623 | | |
| NMTN-04939 | SAV2396 | narH | 2521691 | 2523250 | 2522748 | | |
| NMTN-02731 | SAV2397 | narG | 2523240 | 2526929 | 2523451 | − | respiratory nitrate reductase alpha chain |
| NMTN-02734 | SAV2397 | narG | 2523240 | 2526929 | 2523451 | | |
| NMTN-12489 | SAV2397 | narG | 2523240 | 2526929 | 2523647 | | |
| NMTN-12768 | SAV2397 | narG | 2523240 | 2526929 | 2524116 | | |
| NMTN-01566 | SAV2397 | narG | 2523240 | 2526929 | 2524547 | | |
| NMTN-05440 | SAV2397 | narG | 2523240 | 2526929 | 2524547 | | |
| NMTN-13332 | SAV2397 | narG | 2523240 | 2526929 | 2524547 | | |
| NMTN-00490 | SAV2397 | narG | 2523240 | 2526929 | 2524550 | | |
| NMTN-01710 | SAV2397 | narG | 2523240 | 2526929 | 2525133 | | |
| NMTN-12856 | SAV2397 | narG | 2523240 | 2526929 | 2525408 | | |
| NMTN-11083 | SAV2397 | narG | 2523240 | 2526929 | 2525422 | | |
| NMTN-05625 | SAV2397 | narG | 2523240 | 2526929 | 2525887 | | |
| NMTN-09985 | SAV2397 | narG | 2523240 | 2526929 | 2525988 | | |
| NMTN-11108 | SAV2397 | narG | 2523240 | 2526929 | 2526077 | | |
| NMTN-06924 | SAV2397 | narG | 2523240 | 2526929 | 2526114 | | |
| NMTN-05110 | SAV2397 | narG | 2523240 | 2526929 | 2526601 | | |
| NMTN-14598 | SAV2397 | narG | 2523240 | 2526929 | 2526775 | | |
| NMTN-12701 | SAV2398 | nasF | 2527238 | 2528215 | 2528216 | − | uroporphyrin-III C-methyl transferase |
| NMTN-05367 | SAV2399 | nasE | 2528206 | 2528520 | 2528216 | − | assimilatory nitrite reductase |
| NMTN-02571 | SAV2399 | nasE | 2528206 | 2528520 | 2528219 | | |
| NMTN-09981 | SAV2399 | nasE | 2528206 | 2528520 | 2528269 | | |
| NMTN-02285 | SAV2400 | nasD | 2528524 | 2530929 | 2528719 | − | nitrite reductase |
| NMTN-00457 | SAV2400 | nasD | 2528524 | 2530929 | 2529425 | | |
| NMTN-00535 | SAV2400 | nasD | 2528524 | 2530929 | 2529425 | | |
| NMTN-00829 | SAV2400 | nasD | 2528524 | 2530929 | 2529645 | | |
| NMTN-05082 | SAV2400 | nasD | 2528524 | 2530929 | 2529653 | | |
| NMTN-04784 | SAV2400 | nasD | 2528524 | 2530929 | 2529828 | | |
| NMTN-06612 | SAV2400 | nasD | 2528524 | 2530929 | 2529831 | | |
| NMTN-10956 | SAV2400 | nasD | 2528524 | 2530929 | 2529946 | | |
| NMTN-13822 | SAV2400 | nasD | 2528524 | 2530929 | 2530043 | | |
| NMTN-04087 | SAV2400 | nasD | 2528524 | 2530929 | 2530325 | | |
| NMTN-00448 | SAV2400 | nasD | 2528524 | 2530929 | 2530338 | | |
| NMTN-13849 | SAV2400 | nasD | 2528524 | 2530929 | 2530424 | | |
| NMTN-04629 | SAV2400 | nasD | 2528524 | 2530929 | 2530742 | | |
| NMTN-02449 | SAV2401 | | 2530995 | 2531726 | 2531630 | − | hypothetical protein |
| NMTN-05301 | SAV2404 | | 2533767 | 2533949 | 2533774 | − | hypothetical protein |
| NMTN-05127 | SAV2404 | | 2533767 | 2533949 | 2533912 | | |
| NMTN-00423 | SAV2405 | | 2534033 | 2534500 | 2534163 | − | conserved hypothetical protein |
| NMTN-15346 | SAV2405 | | 2534033 | 2534500 | 2534244 | | |
| NMTN-00011 | SAV2406 | | 2534686 | 2536233 | 2535052 | − | hypothetical protein |
| NMTN-03564 | SAV2406 | | 2534686 | 2536233 | 2535602 | | |
| NMTN-05619 | SAV2406 | | 2534686 | 2536233 | 2535758 | | |
| NMTN-08315 | SAV2408 | | 2536794 | 2537045 | 2536930 | − | conserved hypothetical protein |
| NMTN-04656 | SAV2409 | | 2537318 | 2537917 | 2537582 | − | conserved hypothetical protein |
| NMTN-00671 | SAV2409 | | 2537318 | 2537917 | 2537728 | | |
| NMTN-02970 | SAV2409 | | 2537318 | 2537917 | 2537728 | | |
| NMTN-09559 | SAV2409 | | 2537318 | 2537917 | 2537783 | | |
| NMTN-10967 | SAV2410 | | 2537936 | 2538298 | 2538118 | − | hypothetical protein |
| NMTN-12387 | SAV2410 | | 2537936 | 2538298 | 2538119 | | |
| NMTN-07573 | SAV2410 | | 2537936 | 2538298 | 2538262 | | |
| NMTN-02665 | SAV2411 | fmhA | 2538553 | 2539803 | 2539107 | + | fmhA protein |
| NMTN-09776 | SAV2411 | fmhA | 2538553 | 2539803 | 2539741 | | |
| NMTN-10985 | SAV2411 | fmhA | 2538553 | 2539803 | 2539779 | | |
| NMTN-05574 | SAV2412 | | 2539900 | 2540631 | 2540458 | − | hypothetical protein |
| NMTN-10833 | SAV2414 | | 2541328 | 2542107 | 2542013 | − | hypothetical protein |
| NMTN-05357 | SAV2415 | | 2542228 | 2543667 | 2542632 | − | hypothetical protein |
| NMTN-03616 | SAV2415 | | 2542228 | 2543667 | 2543055 | | |
| NMTN-12359 | SAV2415 | | 2542228 | 2543667 | 2543132 | | |
| NMTN-12181 | SAV2416 | | 2544099 | 2544785 | 2544286 | − | phosphoglycerate mutase |
| NMTN-01732 | SAV2416 | | 2544099 | 2544785 | 2544496 | | |
| NMTN-01483 | SAV2417 | | 2545112 | 2545978 | 2545843 | − | conserved hypothetical protein |
| NMTN-03151 | SAV2418 | sbi | 2546792 | 2548078 | 2546856 | + | IgG-binding protein |
| NMTN-09075 | SAV2418 | sbi | 2546792 | 2548078 | 2547753 | | |
| NMTN-00267 | SAV2418 | sbi | 2546792 | 2548078 | 2547919 | | |
| NMTN-07312 | SAV2419 | hlgA | 2548614 | 2549543 | 2549090 | + | gamma-hemolysin chain II precursor |
| NMTN-05500 | SAV2420 | hlgC | 2550110 | 2551057 | 2550530 | + | gamma-hemolysin component C |
| NMTN-11423 | SAV2421 | hlgB | 2551059 | 2552036 | 2551951 | + | gamma-hemolysin component B |
| NMTN-07727 | SAV2422 | | 2552088 | 2552555 | 2552222 | − | hypothetical protein |
| NMTN-09907 | SAV2422 | | 2552088 | 2552555 | 2552321 | | |
| NMTN-13739 | SAV2424 | | 2553269 | 2554384 | 2553300 | − | hypothetical protein |
| NMTN-08368 | SAV2424 | | 2553269 | 2554384 | 2553343 | | |
| NMTN-07531 | SAV2424 | | 2553269 | 2554384 | 2553455 | | |
| NMTN-03643 | SAV2424 | | 2553269 | 2554384 | 2553528 | | |
| NMTN-10204 | SAV2424 | | 2553269 | 2554384 | 2553716 | | |
| NMTN-13142 | SAV2424 | | 2553269 | 2554384 | 2553947 | | |
| NMTN-09343 | SAV2424 | | 2553269 | 2554384 | 2553949 | | |
| NMTN-01444 | SAV2424 | | 2553269 | 2554384 | 2553990 | | |
| NMTN-01761 | SAV2424 | | 2553269 | 2554384 | 2554089 | | |
| NMTN-04521 | SAV2424 | | 2553269 | 2554384 | 2554101 | | |
| NMTN-04986 | SAV2425 | bioB | 2554362 | 2555369 | 2554544 | − | biotin synthase |
| NMTN-13648 | SAV2425 | bioB | 2554362 | 2555369 | 2554789 | | |
| NMTN-00654 | SAV2425 | bioB | 2554362 | 2555369 | 2554891 | | |
| NMTN-12859d | SAV2426 | bioA | 2555371 | 2556729 | 2555973 | − | adenosylmethionine-8-amino-7- |
| | | | | | | | oxononanoate aminotransferase |
| NMTN-12809 | SAV2426 | bioA | 2555371 | 2556729 | 2556539 | | |
| NMTN-12770t | SAV2426 | bioA | 2555371 | 2556729 | 2556653 | | |
| NMTN-09971 | SAV2427 | bioD | 2556707 | 2557393 | 2557071 | − | dethiobiotin synthetase |
| NMTN-04957 | SAV2428 | | 2557846 | 2559579 | 2557967 | − | hypothetical protein |
| NMTN-05313 | SAV2428 | | 2557846 | 2559579 | 2558456 | | |
| NMTN-11096 | SAV2428 | | 2557846 | 2559579 | 2558572 | | |
| NMTN-02292 | SAV2428 | | 2557846 | 2559579 | 2558741 | | |
| NMTN-04634 | SAV2428 | | 2557846 | 2559579 | 2558777 | | |
| NMTN-07859 | SAV2428 | | 2557846 | 2559579 | 2559233 | | |
| NMTN-07841 | SAV2428 | | 2557846 | 2559579 | 2559353 | | |
| NMTN-05360 | SAV2428 | | 2557846 | 2559579 | 2559421 | | |
| NMTN-12722 | SAV2429 | | 2559604 | 2561367 | 2559602 | − | hypothetical protein |
| NMTN-06331 | SAV2429 | | 2559604 | 2561367 | 2559639 | | |
| NMTN-15221d | SAV2429 | | 2559604 | 2561367 | 2560214 | | |
| NMTN-12818 | SAV2429 | | 2559604 | 2561367 | 2560506 | | |
| NMTN-07187 | SAV2429 | | 2559604 | 2561367 | 2561209 | | |
| NMTN-11692 | SAV2432 | | 2562965 | 2564107 | 2563161 | + | conserved hypothetical protein |
| NMTN-01542 | SAV2433 | | 2564167 | 2564826 | 2564301 | + | hypothetical protein |
| NMTN-12398 | SAV2433 | | 2564167 | 2564826 | 2564513 | | |
| NMTN-04828 | SAV2434 | | 2565008 | 2566219 | 2565098 | + | hypothetical protein |
| NMTN-11807 | SAV2434 | | 2565008 | 2566219 | 2565102 | | |
| NMTN-04538 | SAV2434 | | 2565008 | 2566219 | 2565534 | | |
| NMTN-09530 | SAV2434 | | 2565008 | 2566219 | 2566098 | | |
| NMTN-07737 | SAV2434 | | 2565008 | 2566219 | 2566132 | | |
| NMTN-08352 | SAV2434 | | 2565008 | 2566219 | 2566144 | | |
| NMTN-12445 | SAV2434 | | 2565008 | 2566219 | 2566229 | | |
| NMTN-07043 | SAV2436 | | 2566928 | 2567584 | 2567117 | + | hypothetical protein |
| NMTN-03511 | SAV2436 | | 2566928 | 2567584 | 2567167 | | |
| NMTN-00312 | SAV2436 | | 2566928 | 2567584 | 2567171 | | |
| NMTN-00314 | SAV2436 | | 2566928 | 2567584 | 2567171 | | |
| NMTN-06150 | SAV2436 | | 2566928 | 2567584 | 2567274 | | |
| NMTN-00144 | SAV2436 | | 2566928 | 2567584 | 2567277 | | |
| NMTN-12144 | SAV2436 | | 2566928 | 2567584 | 2567317 | | |
| NMTN-09139 | SAV2436 | | 2566928 | 2567584 | 2567429 | | |
| NMTN-08723 | SAV2437 | | 2567647 | 2568453 | 2567818 | − | conserved hypothetical protein |
| NMTN-08724 | SAV2437 | | 2567647 | 2568453 | 2567818 | | |
| NMTN-08725 | SAV2437 | | 2567647 | 2568453 | 2567818 | | |
| NMTN-08735 | SAV2437 | | 2567647 | 2568453 | 2567818 | | |
| NMTN-08785 | SAV2437 | | 2567647 | 2568453 | 2567818 | | |
| NMTN-08786 | SAV2437 | | 2567647 | 2568453 | 2567818 | | |
| NMTN-08787 | SAV2437 | | 2567647 | 2568453 | 2567818 | | |
| NMTN-08788 | SAV2437 | | 2567647 | 2568453 | 2567818 | | |
| NMTN-08789 | SAV2437 | | 2567647 | 2568453 | 2567818 | | |
| NMTN-08790 | SAV2437 | | 2567647 | 2568453 | 2567818 | | |
| NMTN-08791 | SAV2437 | | 2567647 | 2568453 | 2567818 | | |
| NMTN-08793 | SAV2437 | | 2567647 | 2568453 | 2567818 | | |
| NMTN-08799 | SAV2437 | | 2567647 | 2568453 | 2567818 | | |
| NMTN-08801 | SAV2437 | | 2567647 | 2568453 | 2567818 | | |
| NMTN-08803 | SAV2437 | | 2567647 | 2568453 | 2567818 | | |
| NMTN-08804 | SAV2437 | | 2567647 | 2568453 | 2567818 | | |
| NMTN-08805 | SAV2437 | | 2567647 | 2568453 | 2567818 | | |
| NMTN-08806 | SAV2437 | | 2567647 | 2568453 | 2567818 | | |
| NMTN-08807 | SAV2437 | | 2567647 | 2568453 | 2567818 | | |
| NMTN-08808 | SAV2437 | | 2567647 | 2568453 | 2567818 | | |
| NMTN-08809 | SAV2437 | | 2567647 | 2568453 | 2567818 | | |
| NMTN-08810 | SAV2437 | | 2567647 | 2568453 | 2567818 | | |
| NMTN-08816 | SAV2437 | | 2567647 | 2568453 | 2567818 | | |
| NMTN-08837d | SAV2437 | | 2567647 | 2568453 | 2567818 | | |
| NMTN-08842c | SAV2437 | | 2567647 | 2568453 | 2567818 | | |
| NMTN-08845 | SAV2437 | | 2567647 | 2568453 | 2567818 | | |
| NMTN-08846 | SAV2437 | | 2567647 | 2568453 | 2567818 | | |
| NMTN-08849 | SAV2437 | | 2567647 | 2568453 | 2567818 | | |
| NMTN-08892 | SAV2437 | | 2567647 | 2568453 | 2567818 | | |
| NMTN-06528 | SAV2437 | | 2567647 | 2568453 | 2568108 | | |
| NMTN-13871 | SAV2437 | | 2567647 | 2568453 | 2568263 | | |
| NMTN-02428 | SAV2437 | | 2567647 | 2568453 | 2568264 | | |
| NMTN-03152 | SAV2438 | | 2568739 | 2570148 | 2569760 | + | amino add ABC transporter homolog |
| NMTN-03550 | SAV2438 | | 2568739 | 2570148 | 2569804 | | |
| NMTN-14476 | SAV2438 | | 2568739 | 2570148 | 2569876 | | |
| NMTN-05507 | SAV2438 | | 2568739 | 2570148 | 2569907 | | |
| NMTN-07801 | SAV2438 | | 2568739 | 2570148 | 2570063 | | |
| NMTN-04415 | SAV2439 | | 2570287 | 2572365 | 2570854 | + | hypothetical protein |
| NMTN-03085 | SAV2439 | | 2570287 | 2572365 | 2571088 | | |
| NMTN-04386 | SAV2439 | | 2570287 | 2572365 | 2571164 | | |
| NMTN-11389 | SAV2439 | | 2570287 | 2572365 | 2571437 | | |
| NMTN-10543 | SAV2439 | | 2570287 | 2572365 | 2571773 | | |
| NMTN-07576 | SAV2440 | | 2572506 | 2574335 | 2572591 | − | conserved hypothetical protein |
| NMTN-07812 | SAV2440 | | 2572506 | 2574335 | 2572651 | | |
| NMTN-07120 | SAV2440 | | 2572506 | 2574335 | 2572674 | | |
| NMTN-10025 | SAV2440 | | 2572506 | 2574335 | 2573498 | | |
| NMTN-07875 | SAV2440 | | 2572506 | 2574335 | 2573829 | | |
| NMTN-04529 | SAV2441 | | 2574567 | 2576063 | 2574633 | + | hypothetical protein |
| NMTN-08695 | SAV2441 | | 2574567 | 2576063 | 2574637 | | |
| NMTN-05172 | SAV2441 | | 2574567 | 2576063 | 2574638 | | |
| NMTN-08150 | SAV2441 | | 2574567 | 2576063 | 2574639 | | |
| NMTN-05097 | SAV2441 | | 2574567 | 2576063 | 2574642 | | |
| NMTN-13742 | SAV2441 | | 2574567 | 2576063 | 2574648 | | |
| NMTN-00030 | SAV2441 | | 2574567 | 2576063 | 2574781 | | |
| NMTN-04889 | SAV2441 | | 2574567 | 2576063 | 2575017 | | |
| NMTN-04941 | SAV2441 | | 2574567 | 2576063 | 2575018 | | |
| NMTN-13461 | SAV2441 | | 2574567 | 2576063 | 2575021 | | |
| NMTN-13273 | SAV2441 | | 2574567 | 2576063 | 2575023 | | |
| NMTN-11170 | SAV2441 | | 2574567 | 2576063 | 2575234 | | |
| NMTN-14465 | SAV2441 | | 2574567 | 2576063 | 2575245 | | |
| NMTN-09042 | SAV2441 | | 2574567 | 2576063 | 2575362 | | |
| NMTN-09394 | SAV2441 | | 2574567 | 2576063 | 2575663 | | |
| NMTN-13191 | SAV2441 | | 2574567 | 2576063 | 2575665 | | |
| NMTN-10962 | SAV2441 | | 2574567 | 2576063 | 2575779 | | |
| NMTN-02352 | SAV2441 | | 2574567 | 2576063 | 2575782 | | |
| NMTN-02548 | SAV2441 | | 2574567 | 2576063 | 2575876 | | |
| NMTN-02914 | SAV2441 | | 2574567 | 2576063 | 2575876 | | |
| NMTN-03117 | SAV2441 | | 2574567 | 2576063 | 2575876 | | |
| NMTN-05354 | SAV2441 | | 2574567 | 2576063 | 2575977 | | |
| NMTN-15322 | SAV2442 | | 2576155 | 2577006 | 2576506 | − | hypothetical protein |
| NMTN-11498 | SAV2442 | | 2576155 | 2577006 | 2576750 | | |
| NMTN-05078 | SAV2443 | | 2577328 | 2578263 | 2577916 | − | hypothetical protein |
| NMTN-03490 | SAV2444 | | 2578489 | 2579889 | 2578745 | − | hypothetical protein |
| NMTN-08390 | SAV2444 | | 2578489 | 2579889 | 2578746 | | |
| NMTN-12198 | SAV2444 | | 2578489 | 2579889 | 2579002 | | |
| NMTN-07498 | SAV2445 | opuCD | 2580183 | 2580878 | 2580629 | − | probable glycine |
| | | | | | | | betaine/carnitine/choline ABC |
| | | | | | | | transporter |
| NMTN-04878 | SAV2445 | opuCD | 2580183 | 2580878 | 2580697 | | |
| NMTN-11649 | SAV2446 | opuCC | 2580878 | 2581819 | 2581312 | − | glycine betaine/carnitine/choline ABC |
| | | | | | | | transporter |
| NMTN-00085 | SAV2446 | opuCC | 2580878 | 2581819 | 2581400 | | |
| NMTN-12081 | SAV2446 | opuCC | 2580878 | 2581819 | 2581737 | | |
| NMTN-12575 | SAV2446 | opuCC | 2580878 | 2581819 | 2581737 | | |
| NMTN-04718 | SAV2446 | opuCC | 2580878 | 2581819 | 2581741 | | |
| NMTN-07417 | SAV2447 | opuCB | 2581836 | 2582471 | 2581907 | − | probable glycine |
| | | | | | | | betaine/carnitine/choline ABC |
| | | | | | | | transporter |
| NMTN-14285 | SAV2448 | opuCA | 2582468 | 2583700 | 2582821 | − | glycine betaine/carnitine/choline ABC |
| | | | | | | | transporter |
| NMTN-11368 | SAV2449 | | 2584364 | 2584963 | 2584464 | − | conserved hypothetical protein |
| NMTN-11763 | SAV2449 | | 2584364 | 2584963 | 2584464 | | |
| NMTN-04078 | SAV2449 | | 2584364 | 2584963 | 2584489 | | |
| NMTN-13491 | SAV2449 | | 2584364 | 2584963 | 2584610 | | |
| NMTN-11648 | SAV2450 | | 2585143 | 2586780 | 2585305 | + | hypothetical protein |
| NMTN-04825 | SAV2450 | | 2585143 | 2586780 | 2585387 | | |
| NMTN-06349 | SAV2450 | | 2585143 | 2586780 | 2585481 | | |
| NMTN-09926 | SAV2450 | | 2585143 | 2586780 | 2585610 | | |
| NMTN-12548 | SAV2450 | | 2585143 | 2586780 | 2585650 | | |
| NMTN-10811 | SAV2450 | | 2585143 | 2586780 | 2585895 | | |
| NMTN-08647 | SAV2450 | | 2585143 | 2586780 | 2585909 | | |
| NMTN-08775 | SAV2450 | | 2585143 | 2586780 | 2586780 | | |
| NMTN-08981 | SAV2451 | | 2587089 | 2588441 | 2587251 | + | hypothetical protein |
| NMTN-08781 | SAV2451 | | 2587089 | 2588441 | 2588295 | | |
| NMTN-06100 | SAV2452 | | 2588503 | 2589690 | 2588534 | − | hypothetical protein |
| NMTN-12775 | SAV2452 | | 2588503 | 2589690 | 2588590 | | |
| NMTN-12664 | SAV2452 | | 2588503 | 2589690 | 2588591 | | |
| NMTN-13230 | SAV2452 | | 2588503 | 2589690 | 2589158 | | |
| NMTN-12257 | SAV2452 | | 2588503 | 2589690 | 2589282 | | |
| NMTN-07413 | SAV2452 | | 2588503 | 2589690 | 2589497 | | |
| NMTN-11609 | SAV2453 | | 2590086 | 2590862 | 2590247 | − | conserved hypothetical protein |
| NMTN-13082 | SAV2453 | | 2590086 | 2590862 | 2590414 | | |
| NMTN-03016 | SAV2453 | | 2590086 | 2590862 | 2590579 | | |
| NMTN-03485 | SAV2453 | | 2590086 | 2590862 | 2590579 | | |
| NMTN-03487 | SAV2453 | | 2590086 | 2590862 | 2590579 | | |
| NMTN-04174 | SAV2453 | | 2590086 | 2590862 | 2590630 | | |
| NMTN-06326 | SAV2454 | | 2590855 | 2591517 | 2591323 | − | hypothetical protein |
| NMTN-06334 | SAV2454 | | 2590855 | 2591517 | 2591323 | | |
| NMTN-00440 | SAV2454 | | 2590855 | 2591517 | 2591415 | | |
| NMTN-13218 | SAV2454 | | 2590855 | 2591517 | 2591468 | | |
| NMTN-13294 | SAV2455 | | 2591765 | 2592841 | 2591934 | − | hypothetical protein |
| NMTN-11161 | SAV2455 | | 2591765 | 2592841 | 2591980 | | |
| NMTN-08665 | SAV2455 | | 2591765 | 2592841 | 2592045 | | |
| NMTN-10586 | SAV2455 | | 2591765 | 2592841 | 2592111 | | |
| NMTN-07464 | SAV2455 | | 2591765 | 2592841 | 2592271 | | |
| NMTN-05359 | SAV2455 | | 2591765 | 2592841 | 2592372 | | |
| NMTN-09312 | SAV2457 | | 2593463 | 2593720 | 2593679 | − | hypothetical protein |
| NMTN-05524 | SAV2458 | | 2594079 | 2594534 | 2594329 | + | conserved hypothetical protein |
| NMTN-03535 | SAV2459 | | 2594702 | 2596279 | 2594852 | − | hypothetical protein |
| NMTN-13892 | SAV2459 | | 2594702 | 2596279 | 2595225 | | |
| NMTN-08251 | SAV2459 | | 2594702 | 2596279 | 2595772 | | |
| NMTN-00743 | SAV2459 | | 2594702 | 2596279 | 2596179 | | |
| NMTN-06183 | SAV2459 | | 2594702 | 2596279 | 2596215 | | |
| NMTN-04845 | SAV2459 | | 2594702 | 2596279 | 2596248 | | |
| NMTN-08545 | SAV2460 | | 2596474 | 2597223 | 2596501 | + | hypothetical protein |
| NMTN-11616 | SAV2460 | | 2596474 | 2597223 | 2596772 | | |
| NMTN-09040 | SAV2460 | | 2596474 | 2597223 | 2597087 | | |
| NMTN-12372 | SAV2460 | | 2596474 | 2597223 | 2597139 | | |
| NMTN-15193 | SAV2462 | | 2597585 | 2598778 | 2598103 | − | hypothetical protein |
| NMTN-12341 | SAV2462 | | 2597585 | 2598778 | 2598385 | | |
| NMTN-11376 | SAV2462 | | 2597585 | 2598778 | 2598670 | | |
| NMTN-03046 | SAV2462 | | 2597585 | 2598778 | 2598739 | | |
| NMTN-03047 | SAV2462 | | 2597585 | 2598778 | 2598739 | | |
| NMTN-03052 | SAV2462 | | 2597585 | 2598778 | 2598739 | | |
| NMTN-03057 | SAV2462 | | 2597585 | 2598778 | 2598739 | | |
| NMTN-03065 | SAV2462 | | 2597585 | 2598778 | 2598739 | | |
| NMTN-04005 | SAV2463 | | 2598790 | 2599539 | 2598827 | − | oligopeptide transporter putative |
| | | | | | | | ATPase domain |
| NMTN-07837 | SAV2463 | | 2598790 | 2599539 | 2598932 | | |
| NMTN-07864 | SAV2463 | | 2598790 | 2599539 | 2598974 | | |
| NMTN-05105 | SAV2463 | | 2598790 | 2599539 | 2599371 | | |
| NMTN-06696 | SAV2463 | | 2598790 | 2599539 | 2599375 | | |
| NMTN-09136 | SAV2464 | | 2599532 | 2600347 | 2599833 | − | oligopeptide transporter putative |
| | | | | | | | ATPase domain |
| NMTN-08046 | SAV2464 | | 2599532 | 2600347 | 2599955 | | |
| NMTN-03694 | SAV2464 | | 2599532 | 2600347 | 2600001 | | |
| NMTN-13066 | SAV2464 | | 2599532 | 2600347 | 2600001 | | |
| NMTN-01677 | SAV2465 | | 2600344 | 2601213 | 2600867 | − | oligopeptide transporter putative |
| | | | | | | | membrane permease domain |
| NMTN-12978 | SAV2465 | | 2600344 | 2601213 | 2600870 | | |
| NMTN-11728 | SAV2465 | | 2600344 | 2601213 | 2601098 | | |
| NMTN-02472 | SAV2466 | | 2601210 | 2602145 | 2601239 | − | oligopeptide transporter putative |
| | | | | | | | membrane permease domain |
| NMTN-05350 | SAV2466 | | 2601210 | 2602145 | 2601516 | | |
| NMTN-04010 | SAV2466 | | 2601210 | 2602145 | 2601568 | | |
| NMTN-10589 | SAV2466 | | 2601210 | 2602145 | 2601891 | | |
| NMTN-13633 | SAV2466 | | 2601210 | 2602145 | 2601895 | | |
| NMTN-10778 | SAV2467 | | 2602158 | 2603756 | 2602166 | − | oligopeptide transporter putative |
| | | | | | | | substrate binding domain |
| NMTN-14290 | SAV2467 | | 2602158 | 2603756 | 2602494 | | |
| NMTN-02792 | SAV2467 | | 2602158 | 2603756 | 2602580 | | |
| NMTN-07110 | SAV2467 | | 2602158 | 2603756 | 2602728 | | |
| NMTN-08100 | SAV2467 | | 2602158 | 2603756 | 2602775 | | |
| NMTN-04194 | SAV2467 | | 2602158 | 2603756 | 2603329 | | |
| NMTN-00595 | SAV2467 | | 2602158 | 2603756 | 2603410 | | |
| NMTN-05145 | SAV2467 | | 2602158 | 2603756 | 2603629 | − | conserved hypothetical protein |
| NMTN-13700 | SAV2468 | | 2603899 | 2605200 | 2603987 | | |
| NMTN-04787 | SAV2468 | | 2603899 | 2605200 | 2604742 | | |
| NMTN-10983 | SAV2469 | | 2605193 | 2606011 | 2605495 | − | conserved hypothetical protein |
| NMTN-03991 | SAV2469 | | 2605193 | 2606011 | 2605693 | | |
| NMTN-04552 | SAV2469 | | 2605193 | 2606011 | 2605696 | | |
| NMTN-09476 | SAV2469 | | 2605193 | 2606011 | 2605859 | | |
| NMTN-12653 | SAV2469 | | 2605193 | 2606011 | 2605936 | | |
| NMTN-03579 | SAV2470 | | 2606022 | 2606843 | 2606027 | − | hypothetical protein |
| NMTN-09320 | SAV2470 | | 2606022 | 2606843 | 2606063 | | |
| NMTN-03768 | SAV2470 | | 2606022 | 2606843 | 2606253 | | |
| NMTN-13551 | SAV2470 | | 2606022 | 2606843 | 2606296 | | |
| NMTN-07780 | SAV2470 | | 2606022 | 2606843 | 2606384 | | |
| NMTN-03928 | SAV2470 | | 2606022 | 2606843 | 2606402 | | |
| NMTN-06926 | SAV2470 | | 2606022 | 2606843 | 2606711 | | |
| NMTN-04362 | SAV2473 | | 2609453 | 2610991 | 2609606 | + | hypothetical protein |
| NMTN-08002 | SAV2473 | | 2609453 | 2610991 | 2609630 | | |
| NMTN-05371 | SAV2473 | | 2609453 | 2610991 | 2609691 | | |
| NMTN-13406 | SAV2473 | | 2609453 | 2610991 | 2610140 | | |
| NMTN-11580 | SAV2473 | | 2609453 | 2610991 | 2610178 | | |
| NMTN-12708 | SAV2473 | | 2609453 | 2610991 | 2610318 | | |
| NMTN-01504 | SAV2473 | | 2609453 | 2610991 | 2610950 | | |
| NMTN-05121 | SAV2473 | | 2609453 | 2610991 | 2610950 | | |
| NMTN-05552 | SAV2476 | | 2612424 | 2612834 | 2612438 | − | hypothetical protein |
| NMTN-00313 | SAV2476 | | 2612424 | 2612834 | 2612695 | | |
| NMTN-11667 | SAV2476 | | 2612424 | 2612834 | 2612800 | | |
| NMTN-04707 | SAV2477 | | 2613101 | 2613613 | 2613267 | − | hypothetical protein |
| NMTN-05047 | SAV2477 | | 2613101 | 2613613 | 2613477 | | |
| NMTN-03921 | SAV2478 | | 2613773 | 2614468 | 2613830 | − | hypothetical protein |
| NMTN-12495 | SAV2478 | | 2613773 | 2614468 | 2613835 | | |
| NMTN-12332 | SAV2478 | | 2613773 | 2614468 | 2614023 | | |
| NMTN-11265 | SAV2478 | | 2613773 | 2614468 | 2614422 | | |
| NMTN-12835 | SAV2479 | | 2614757 | 2615038 | 2614765 | + | hypothetical protein |
| NMTN-02065 | SAV2479 | | 2614757 | 2615038 | 2614853 | | |
| NMTN-06614 | SAV2480 | | 2615298 | 2615489 | 2615378 | − | hypothetical protein |
| NMTN-00282 | SAV2481 | | 2616050 | 2616844 | 2616557 | − | hypothetical protein |
| NMTN-11149 | SAV2483 | | 2617670 | 2617996 | 2617867 | − | hypothetical protein |
| NMTN-09762 | SAV2484 | | 2617996 | 2619321 | 2618605 | − | hypothetical protein |
| NMTN-14535 | SAV2484 | | 2617996 | 2619321 | 2618983 | | |
| NMTN-06333 | SAV2484 | | 2617996 | 2619321 | 2618985 | | |
| NMTN-01846 | SAV2487 | | 2621231 | 2622007 | 2621431 | − | hypothetical protein |
| NMTN-06282 | SAV2487 | | 2621231 | 2622007 | 2621870 | | |
| NMTN-06578 | SAV2487 | | 2621231 | 2622007 | 2621970 | | |
| NMTN-09447 | SAV2488 | | 2622311 | 2623801 | 2622332 | − | conserved hypothetical protein |
| NMTN-11039 | SAV2488 | | 2622311 | 2623801 | 2623003 | | |
| NMTN-01935 | SAV2488 | | 2622311 | 2623801 | 2623170 | | |
| NMTN-07691 | SAV2488 | | 2622311 | 2623801 | 2623225 | | |
| NMTN-00745 | SAV2488 | | 2622311 | 2623801 | 2623227 | | |
| NMTN-00216 | SAV2488 | | 2622311 | 2623801 | 2623275 | | |
| NMTN-00390 | SAV2488 | | 2622311 | 2623801 | 2623275 | | |
| NMTN-03411 | SAV2488 | | 2622311 | 2623801 | 2623390 | | |
| NMTN-02576 | SAV2488 | | 2622311 | 2623801 | 2623664 | | |
| NMTN-02568 | SAV2489 | | 2623841 | 2625172 | 2623984 | − | conserved hypothetical protein |
| NMTN-14766 | SAV2489 | | 2623841 | 2625172 | 2623986 | | |
| NMTN-00582 | SAV2489 | | 2623841 | 2625172 | 2624479 | | |
| NMTN-06424 | SAV2489 | | 2623841 | 2625172 | 2624616 | | |
| NMTN-12174 | SAV2489 | | 2623841 | 2625172 | 2624778 | | |
| NMTN-15357 | SAV2489 | | 2623841 | 2625172 | 2624781 | | |
| NMTN-08751 | SAV2489 | | 2623841 | 2625172 | 2624804 | | |
| NMTN-11196 | SAV2489 | | 2623841 | 2625172 | 2624898 | | |
| NMTN-00239 | SAV2489 | | 2623841 | 2625172 | 2625037 | | |
| NMTN-00306 | SAV2489 | | 2623841 | 2625172 | 2625037 | | |
| NMTN-02020 | SAV2490 | | 2625174 | 2625566 | 2625190 | − | hypothetical protein |
| NMTN-10209 | SAV2492 | | 2627790 | 2628056 | 2627821 | + | hypothetical protein |
| NMTN-03705 | SAV2492 | | 2627790 | 2628056 | 2627959 | | |
| NMTN-05393 | SAV2492 | | 2627790 | 2628056 | 2627959 | | |
| NMTN-11601 | SAV2493 | | 2628200 | 2628508 | 2628350 | + | hypothetical protein |
| NMTN-10624 | SAV2493 | | 2628200 | 2628508 | 2628515 | | |
| NMTN-11084 | SAV2494 | | 2628616 | 2628849 | 2628663 | − | hypothetical protein |
| NMTN-08431 | SAV2494 | | 2628616 | 2628849 | 2628800 | | |
| NMTN-09984 | SAV2494 | | 2628616 | 2628849 | 2628800 | | |
| NMTN-06119 | SAV2495 | | 2628946 | 2630022 | 2629096 | − | conserved hypothetical protein |
| NMTN-04772 | SAV2495 | | 2628946 | 2630022 | 2629256 | | |
| NMTN-04326 | SAV2495 | | 2628946 | 2630022 | 2629257 | | |
| NMTN-06709 | SAV2495 | | 2628946 | 2630022 | 2629282 | | |
| NMTN-09493 | SAV2495 | | 2628946 | 2630022 | 2629530 | | |
| NMTN-14625 | SAV2495 | | 2628946 | 2630022 | 2629802 | | |
| NMTN-12431 | SAV2497 | | 2632418 | 2633752 | 2632605 | − | hypothetical protein |
| NMTN-03242 | SAV2498 | sarH3 | 2634057 | 2634416 | 2634157 | − | staphylococcal accessory regulator A |
| | | | | | | | homolog |
| NMTN-02575 | SAV2499 | sarH2 | 2634737 | 2635480 | 2635232 | + | staphylococcal accessory regulator A |
| | | | | | | | homolog |
| NMTN-01952 | SAV2500 | gtaB | 2635691 | 2636557 | 2635884 | − | UTP-glucose-1-phosphate |
| | | | | | | | uridyltransferase |
| NMTN-02270 | SAV2501 | tnp | 2636730 | 2638049 | 2637083 | − | transposase |
| NMTN-02964 | SAV2501 | tnp | 2636730 | 2638049 | 2637994 | | |
| NMTN-02992 | SAV2502 | fnbB | 2638258 | 2641143 | 2638763 | − | fibronectin-binding protein homolog |
| NMTN-00088 | SAV2502 | fnbB | 2638258 | 2641143 | 2638886 | | |
| NMTN-06235 | SAV2502 | fnbB | 2638258 | 2641143 | 2639739 | | |
| NMTN-00140 | SAV2502 | fnbB | 2638258 | 2641143 | 2639958 | | |
| NMTN-14017 | SAV2502 | fnbB | 2638258 | 2641143 | 2640018 | | |
| NMTN-00627 | SAV2502 | fnbB | 2638258 | 2641143 | 2640424 | | |
| NMTN-05396 | SAV2502 | fnbB | 2638258 | 2641143 | 2640873 | | |
| NMTN-02446 | SAV2503 | fnb | 2641824 | 2644940 | 2644305 | − | fibronectin-binding protein homolog |
| NMTN-02646 | SAV2503 | fnb | 2641824 | 2644940 | 2644523 | | |
| NMTN-11549 | SAV2505 | gntP | 2645409 | 2646767 | 2645660 | − | gluconate permease |
| NMTN-12924 | SAV2505 | gntP | 2645409 | 2646767 | 2646026 | | |
| NMTN-11806 | SAV2505 | gntP | 2645409 | 2646767 | 2646179 | | |
| NMTN-05112 | SAV2505 | gntP | 2645409 | 2646767 | 2646676 | | |
| NMTN-05115 | SAV2505 | gntP | 2645409 | 2646767 | 2646676 | | |
| NMTN-01589 | SAV2506 | gntK | 2646884 | 2648437 | 2647108 | − | gluconokinase |
| NMTN-03786 | SAV2506 | gntK | 2646884 | 2648437 | 2647460 | | |
| NMTN-00576 | SAV2506 | gntK | 2646884 | 2648437 | 2647542 | | |
| NMTN-09486 | SAV2506 | gntK | 2646884 | 2648437 | 2648043 | | |
| NMTN-02437 | SAV2507 | gntR | 2648461 | 2649141 | 2648542 | − | gluconate operon transcriptional |
| | | | | | | | repressor |
| NMTN-14622 | SAV2507 | gntR | 2648461 | 2649141 | 2649133 | | |
| NMTN-05616 | SAV2508 | | 2649294 | 2650058 | 2649548 | − | hypothetical protein |
| NMTN-02984 | SAV2508 | | 2649294 | 2650058 | 2649863 | | |
| NMTN-12801 | SAV2508 | | 2649294 | 2650058 | 2650050 | | |
| NMTN-09462 | SAV2509 | | 2650223 | 2650915 | 2650367 | − | hypothetical protein |
| NMTN-05634 | SAV2509 | | 2650223 | 2650915 | 2650500 | | |
| NMTN-10441 | SAV2510 | | 2651146 | 2651634 | 2651218 | − | conserved hypothetical protein |
| NMTN-12937 | SAV2511 | | 2651701 | 2653524 | 2651912 | − | conserved hypothetical protein |
| NMTN-08737 | SAV2511 | | 2651701 | 2653524 | 2651956 | | |
| NMTN-09154 | SAV2511 | | 2651701 | 2653524 | 2651996 | | |
| NMTN-10894 | SAV2511 | | 2651701 | 2653524 | 2652079 | | |
| NMTN-12086 | SAV2511 | | 2651701 | 2653524 | 2652305 | | |
| NMTN-09231 | SAV2511 | | 2651701 | 2653524 | 2652492 | | |
| NMTN-08003 | SAV2511 | | 2651701 | 2653524 | 2652590 | | |
| NMTN-04449 | SAV2511 | | 2651701 | 2653524 | 2652599 | | |
| NMTN-10842 | SAV2511 | | 2651701 | 2653524 | 2652599 | | |
| NMTN-04773 | SAV2511 | | 2651701 | 2653524 | 2652725 | | |
| NMTN-11294 | SAV2511 | | 2651701 | 2653524 | 2652818 | | |
| NMTN-09527 | SAV2511 | | 2651701 | 2653524 | 2653068 | | |
| NMTN-04803 | SAV2511 | | 2651701 | 2653524 | 2653189 | | |
| NMTN-05000 | SAV2511 | | 2651701 | 2653524 | 2653287 | | |
| NMTN-07937 | SAV2512 | | 2654001 | 2655278 | 2654186 | − | hypothetical protein |
| NMTN-02069 | SAV2512 | | 2654001 | 2655278 | 2654300 | | |
| NMTN-05605 | SAV2512 | | 2654001 | 2655278 | 2654304 | | |
| NMTN-11594 | SAV2512 | | 2654001 | 2655278 | 2654543 | | |
| NMTN-12915 | SAV2512 | | 2654001 | 2655278 | 2654931 | | |
| NMTN-00223 | SAV2513 | | 2655825 | 2656436 | 2655904 | + | hypothetical protein |
| NMTN-10052 | SAV2515 | | 2657651 | 2658421 | 2657730 | + | hypothetical protein |
| NMTN-14411 | SAV2515 | | 2657651 | 2658421 | 2657735 | | |
| NMTN-14496 | SAV2515 | | 2657651 | 2658421 | 2657852 | | |
| NMTN-13631 | SAV2515 | | 2657651 | 2658421 | 2657853 | | |
| NMTN-13923 | SAV2515 | | 2657651 | 2658421 | 2657853 | | |
| NMTN-13925 | SAV2515 | | 2657651 | 2658421 | 2657853 | | |
| NMTN-13927 | SAV2515 | | 2657651 | 2658421 | 2657853 | | |
| NMTN-13930 | SAV2515 | | 2657651 | 2658421 | 2657853 | | |
| NMTN-13936 | SAV2515 | | 2657651 | 2658421 | 2657853 | | |
| NMTN-13978 | SAV2515 | | 2657651 | 2658421 | 2657853 | | |
| NMTN-14368 | SAV2515 | | 2657651 | 2658421 | 2657853 | | |
| NMTN-14492 | SAV2515 | | 2657651 | 2658421 | 2657853 | | |
| NMTN-14493 | SAV2515 | | 2657651 | 2658421 | 2657853 | | |
| NMTN-14494 | SAV2515 | | 2657651 | 2658421 | 2657853 | | |
| NMTN-14495 | SAV2515 | | 2657651 | 2658421 | 2657853 | | |
| NMTN-14575 | SAV2515 | | 2657651 | 2658421 | 2657853 | | |
| NMTN-14576 | SAV2515 | | 2657651 | 2658421 | 2657853 | | |
| NMTN-14577 | SAV2515 | | 2657651 | 2658421 | 2657853 | | |
| NMTN-14578 | SAV2515 | | 2657651 | 2658421 | 2657853 | | |
| NMTN-14579 | SAV2515 | | 2657651 | 2658421 | 2657853 | | |
| NMTN-14580 | SAV2515 | | 2657651 | 2658421 | 2657853 | | |
| NMTN-14582 | SAV2515 | | 2657651 | 2658421 | 2657853 | | |
| NMTN-14584 | SAV2515 | | 2657651 | 2658421 | 2657853 | | |
| NMTN-14586 | SAV2515 | | 2657651 | 2658421 | 2657853 | | |
| NMTN-14587 | SAV2515 | | 2657651 | 2658421 | 2657853 | | |
| NMTN-14591 | SAV2515 | | 2657651 | 2658421 | 2657853 | | |
| NMTN-14667 | SAV2515 | | 2657651 | 2658421 | 2657853 | | |
| NMTN-14668 | SAV2515 | | 2657651 | 2658421 | 2657853 | | |
| NMTN-14669 | SAV2515 | | 2657651 | 2658421 | 2657853 | | |
| NMTN-14726 | SAV2515 | | 2657651 | 2658421 | 2657853 | | |
| NMTN-14728 | SAV2515 | | 2657651 | 2658421 | 2657853 | | |
| NMTN-14729 | SAV2515 | | 2657651 | 2658421 | 2657853 | | |
| NMTN-14733 | SAV2515 | | 2657651 | 2658421 | 2657853 | | |
| NMTN-14734 | SAV2515 | | 2657651 | 2658421 | 2657853 | | |
| NMTN-14735 | SAV2515 | | 2657651 | 2658421 | 2657853 | | |
| NMTN-14736 | SAV2515 | | 2657651 | 2658421 | 2657853 | | |
| NMTN-14737 | SAV2515 | | 2657651 | 2658421 | 2657853 | | |
| NMTN-14738 | SAV2515 | | 2657651 | 2658421 | 2657853 | | |
| NMTN-14740 | SAV2515 | | 2657651 | 2658421 | 2657853 | | |
| NMTN-14741 | SAV2515 | | 2657651 | 2658421 | 2657853 | | |
| NMTN-14743 | SAV2515 | | 2657651 | 2658421 | 2657853 | | |
| NMTN-14745 | SAV2515 | | 2657651 | 2658421 | 2657853 | | |
| NMTN-14746 | SAV2515 | | 2657651 | 2658421 | 2657853 | | |
| NMTN-14748 | SAV2515 | | 2657651 | 2658421 | 2657853 | | |
| NMTN-14752 | SAV2515 | | 2657651 | 2658421 | 2657853 | | |
| NMTN-14754 | SAV2515 | | 2657651 | 2658421 | 2657853 | | |
| NMTN-14775 | SAV2515 | | 2657651 | 2658421 | 2657853 | | |
| NMTN-14785 | SAV2515 | | 2657651 | 2658421 | 2657853 | | |
| NMTN-14882 | SAV2515 | | 2657651 | 2658421 | 2657853 | | |
| NMTN-14899 | SAV2515 | | 2657651 | 2658421 | 2657853 | | |
| NMTN-14900 | SAV2515 | | 2657651 | 2658421 | 2657853 | | |
| NMTN-14900 | SAV2515 | | 2657651 | 2658421 | 2657853 | | |
| NMTN-14969 | SAV2515 | | 2657651 | 2658421 | 2657853 | | |
| NMTN-15079 | SAV2515 | | 2657651 | 2658421 | 2657853 | | |
| NMTN-15083 | SAV2515 | | 2657651 | 2658421 | 2657853 | | |
| NMTN-15089 | SAV2515 | | 2657651 | 2658421 | 2657853 | | |
| NMTN-15093 | SAV2515 | | 2657651 | 2658421 | 2657853 | | |
| NMTN-15099 | SAV2515 | | 2657651 | 2658421 | 2657853 | | |
| NMTN-15106 | SAV2515 | | 2657651 | 2658421 | 2657853 | | |
| NMTN-15144 | SAV2515 | | 2657651 | 2658421 | 2657853 | | |
| NMTN-14750 | SAV2515 | | 2657651 | 2658421 | 2657854 | | |
| NMTN-15141 | SAV2515 | | 2657651 | 2658421 | 2657854 | | |
| NMTN-10994 | SAV2515 | | 2657651 | 2658421 | 2658080 | | |
| NMTN-08143 | SAV2516 | fbp | 2658794 | 2660758 | 2658979 | + | fructose-bisphosphatase |
| NMTN-01975 | SAV2516 | fbp | 2658794 | 2660758 | 2659350 | | |
| NMTN-02338 | SAV2516 | fbp | 2658794 | 2660758 | 2659350 | | |
| NMTN-06609 | SAV2516 | fbp | 2658794 | 2660758 | 2659873 | | |
| NMTN-02774 | SAV2516 | fbp | 2658794 | 2660758 | 2660044 | | |
| NMTN-02445 | SAV2516 | fbp | 2658794 | 2660758 | 2660275 | | |
| NMTN-02463 | SAV2516 | fbp | 2658794 | 2660758 | 2660335 | | |
| NMTN-10881 | SAV2517 | | 2661100 | 2662179 | 2661170 | + | conserved hypothetical protein |
| NMTN-03402 | SAV2517 | | 2661100 | 2662179 | 2661853 | | |
| NMTN-12163 | SAV2517 | | 2661100 | 2662179 | 2662181 | | |
| NMTN-05603 | SAV2518 | | 2662284 | 2662877 | 2662552 | − | conserved hypothetical protein |
| NMTN-11951 | SAV2519 | | 2662908 | 2663867 | 2663088 | − | hypothetical protein |
| NMTN-13391d | SAV2519 | | 2662908 | 2663867 | 2663338 | | |
| NMTN-09546 | SAV2519 | | 2662908 | 2663867 | 2663733 | | |
| NMTN-13650 | SAV2519 | | 2662908 | 2663867 | 2663774 | | |
| NMTN-04104 | SAV2519 | | 2662908 | 2663867 | 2663861 | | |
| NMTN-11239 | SAV2520 | | 2663945 | 2664379 | 2664106 | − | hypothetical protein |
| NMTN-02392 | SAV2520 | | 2663945 | 2664379 | 2664286 | | |
| NMTN-06196 | SAV2521 | | 2664605 | 2664889 | 2664603 | + | conserved hypothetical protein |
| NMTN-13298 | SAV2521 | | 2664605 | 2664889 | 2664606 | | |
| NMTN-10416 | SAV2522 | | 2665060 | 2665866 | 2665279 | + | conserved hypothetical protein |
| NMTN-06488 | SAV2522 | | 2665060 | 2665866 | 2665573 | | |
| NMTN-11704 | SAV2522 | | 2665060 | 2665866 | 2665697 | | |
| NMTN-06870 | SAV2523 | | 2665987 | 2666658 | 2666379 | − | hypothetical protein |
| NMTN-11429 | SAV2523 | | 2665987 | 2666658 | 2666543 | | |
| NMTN-07315 | SAV2524 | ddh | 2666865 | 2667920 | 2667019 | + | D-specific D-2-hydroxyacid |
| | | | | | | | dehydrogenase |
| NMTN-13284 | SAV2525 | | 2668167 | 2668967 | 2668163 | − | conserved hypothetical protein |
| NMTN-13859 | SAV2525 | | 2668167 | 2668967 | 2668417 | | |
| NMTN-13860 | SAV2525 | | 2668167 | 2668967 | 2668417 | | |
| NMTN-08482 | SAV2525 | | 2668167 | 2668967 | 2668722 | | |
| NMTN-03857 | SAV2525 | | 2668167 | 2668967 | 2668801 | | |
| NMTN-03861 | SAV2525 | | 2668167 | 2668967 | 2668801 | | |
| NMTN-09879 | SAV2526 | | 2669287 | 2669922 | 2669414 | + | hypothetical protein |
| NMTN-14514 | SAV2526 | | 2669287 | 2669922 | 2669811 | | |
| NMTN-04500 | SAV2526 | | 2669287 | 2669922 | 2669915 | | |
| NMTN-00284 | SAV2527 | | 2669937 | 2670659 | 2670015 | + | conserved hypothetical protein |
| NMTN-02253 | SAV2527 | | 2669937 | 2670659 | 2670088 | | |
| NMTN-03197 | SAV2527 | | 2669937 | 2670659 | 2670091 | | |
| NMTN-02600 | SAV2527 | | 2669937 | 2670659 | 2670291 | | |
| NMTN-11401 | SAV2527 | | 2669937 | 2670659 | 2670324 | | |
| NMTN-11249 | SAV2527 | | 2669937 | 2670659 | 2670345 | | |
| NMTN-13022 | SAV2527 | | 2669937 | 2670659 | 2670604 | | |
| NMTN-03486 | SAV2528 | srtA | 2670821 | 2671441 | 2670951 | − | sortase |
| NMTN-13599 | SAV2529 | | 2671635 | 2672126 | 2671701 | + | hypothetical protein |
| NMTN-04089 | SAV2529 | | 2671635 | 2672126 | 2671705 | | |
| NMTN-11627 | SAV2529 | | 2671635 | 2672126 | 2671779 | | |
| NMTN-08659 | SAV2530 | | 2672619 | 2673518 | 2673380 | − | hypothetical protein |
| NMTN-01751 | SAV2531/ | | 2673532 | 2674212 | 2673621 | | |
| | SAV922 | | | | | | |
| NMTN-08339 | SAV2531 | | 2673532 | 2674212 | 2673756 | − | hypothetical protein |
| NMTN-03188 | SAV2531 | | 2673532 | 2674212 | 2673791 | | |
| NMTN-06735 | SAV2532 | | 2674215 | 2675258 | 2674564 | − | hypothetical protein |
| NMTN-06563 | SAV2532 | | 2674215 | 2675258 | 2674679 | | |
| NMTN-08308 | SAV2532 | | 2674215 | 2675258 | 2674760 | | |
| NMTN-05330 | SAV2532 | | 2674215 | 2675258 | 2675237 | | |
| NMTN-10747 | SAV2534 | | 2676203 | 2677129 | 2676378 | + | conserved hypothetical protein |
| NMTN-13034 | SAV2534 | | 2676203 | 2677129 | 2676785 | | |
| NMTN-12580 | SAV2534 | | 2676203 | 2677129 | 2676956 | | |
| NMTN-08619 | SAV2535 | | 2677210 | 2678178 | 2677501 | − | conserved hypothetical protein |
| NMTN-00187 | SAV2535 | | 2677210 | 2678178 | 2677522 | | |
| NMTN-12724 | SAV2537 | | 2678697 | 2679107 | 2678866 | − | conserved hypothetical protein |
| NMTN-09292 | SAV2538 | ptsG | 2679487 | 2681553 | 2679810 | − | PTS system, glucose-specific II ABC |
| | | | | | | | component |
| NMTN-05528 | SAV2538 | ptsG | 2679487 | 2681553 | 2680043 | | |
| NMTN-10609 | SAV2538 | ptsG | 2679487 | 2681553 | 2680367 | | |
| NMTN-09373 | SAV2538 | ptsG | 2679487 | 2681553 | 2680581 | | |
| NMTN-09373 | SAV2538 | ptsG | 2679487 | 2681553 | 2680581 | | |
| NMTN-14412 | SAV2538 | ptsG | 2679487 | 2681553 | 2680925 | | |
| NMTN-00211 | SAV2538 | ptsG | 2679487 | 2681553 | 2681096 | | |
| NMTN-00373 | SAV2538 | ptsG | 2679487 | 2681553 | 2681096 | | |
| NMTN-03274 | SAV2538 | ptsG | 2679487 | 2681553 | 2681396 | | |
| NMTN-12306 | SAV2539 | | 2681854 | 2683593 | 2682234 | − | hypothetical protein |
| NMTN-07818 | SAV2539 | | 2681854 | 2683593 | 2683101 | | |
| NMTN-04814 | SAV2539 | | 2681854 | 2683593 | 2683316 | | |
| NMTN-07476 | SAV2539 | | 2681854 | 2683593 | 2683333 | | |
| NMTN-00047 | SAV2539 | | 2681854 | 2683593 | 2683449 | | |
| NMTN-04017 | SAV2539 | | 2681854 | 2683593 | 2683485 | | |
| NMTN-08313 | SAV2539 | | 2681854 | 2683593 | 2683541 | | |
| NMTN-08702 | SAV2540 | | 2683644 | 2684333 | 2683720 | − | conserved hypothetical protein |
| NMTN-11998 | SAV2540 | | 2683644 | 2684333 | 2683844 | | |
| NMTN-12224 | SAV2541 | | 2684326 | 2684721 | 2684445 | − | conserved hypothetical protein |
| NMTN-14640 | SAV2541 | | 2684326 | 2684721 | 2684688 | | |
| NMTN-09433 | SAV2542 | | 2684934 | 2685821 | 2685029 | − | hypothetical protein |
| NMTN-12036 | SAV2542 | | 2684934 | 2685821 | 2685029 | | |
| NMTN-13139 | SAV2542 | | 2684934 | 2685821 | 2685029 | | |
| NMTN-06771d | SAV2542 | | 2684934 | 2685821 | 2685135 | | |
| NMTN-11209 | SAV2542 | | 2684934 | 2685821 | 2685351 | | |
| NMTN-11159 | SAV2542 | | 2684934 | 2685821 | 2685489 | | |
| NMTN-00643 | SAV2542 | | 2684934 | 2685821 | 2685538 | | |
| NMTN-08297 | SAV2542 | | 2684934 | 2685821 | 2685626 | | |
| NMTN-11917 | SAV2543 | | 2685979 | 2686197 | 2686151 | + | hypothetical protein |
| NMTN-06492 | SAV2544 | | 2686587 | 2687018 | 2687010 | + | hypothetical protein |
| NMTN-08964 | SAV2544 | | 2686587 | 2687018 | 2687010 | | |
| NMTN-11357 | SAV2545 | mvaA | 2687157 | 2688434 | 2687881 | − | hydroxymethylglutaryl-CoA reductase |
| NMTN-14634 | SAV2546 | mvaS | 2688689 | 2689855 | 2689263 | + | 3-hydroxy-3-methylglutaryl CoA |
| | | | | | | | synthase |
| NMTN-07991 | SAV2548 | clpL | 2690950 | 2693055 | 2691042 | + | ATP-dependent Clp proteinase chain |
| NMTN-00319 | SAV2548 | clpL | 2690950 | 2693055 | 2691045 | | |
| NMTN-08133 | SAV2548 | clpL | 2690950 | 2693055 | 2691488 | | |
| NMTN-11419 | SAV2548 | clpL | 2690950 | 2693055 | 2691546 | | |
| NMTN-00686 | SAV2548 | clpL | 2690950 | 2693055 | 2692490 | | |
| NMTN-05348 | SAV2548 | clpL | 2690950 | 2693055 | 2693017 | | |
| NMTN-06146 | SAV2548 | clpL | 2690950 | 2693055 | 2693017 | | |
| NMTN-11788 | SAV2548 | clpL | 2690950 | 2693055 | 2693017 | | |
| NMTN-06146 | SAV2548 | clpL | 2690950 | 2693055 | 2693017 | | |
| NMTN-11788 | SAV2548 | clpL | 2690950 | 2693055 | 2693017 | | |
| NMTN-05131 | SAV2549 | | 2693124 | 2693294 | 2693194 | − | hypothetical protein |
| NMTN-05154 | SAV2549 | | 2693124 | 2693294 | 2693194 | | |
| NMTN-05161 | SAV2549 | | 2693124 | 2693294 | 2693194 | | |
| NMTN-04186 | SAV2549 | | 2693124 | 2693294 | 2693236 | | |
| NMTN-08409 | SAV2550 | feoB | 2693307 | 2695301 | 2693392 | − | ferrous iron transport protein B homolog |
| NMTN-04437 | SAV2550 | feoB | 2693307 | 2695301 | 2693448 | | |
| NMTN-09781 | SAV2550 | feoB | 2693307 | 2695301 | 2694204 | | |
| NMTN-12591 | SAV2550 | feoB | 2693307 | 2695301 | 2694346 | | |
| NMTN-14475 | SAV2550 | feoB | 2693307 | 2695301 | 2694792 | | |
| NMTN-10189 | SAV2550 | feoB | 2693307 | 2695301 | 2695071 | | |
| NMTN-13212 | SAV2550 | feoB | 2693307 | 2695301 | 2695135 | | |
| NMTN-04953 | SAV2551 | | 2695313 | 2695540 | 2695386 | − | hypothetical protein |
| NMTN-11912 | SAV2551 | | 2695313 | 2695540 | 2695453 | | |
| NMTN-09037 | SAV2551 | | 2695313 | 2695540 | 2695542 | | |
| NMTN-14609 | SAV2552 | | 2695756 | 2698245 | 2695893 | − | hypothetical protein |
| NMTN-10855 | SAV2552 | | 2695756 | 2698245 | 2696205 | | |
| NMTN-13305 | SAV2552 | | 2695756 | 2698245 | 2697506 | | |
| NMTN-01546 | SAV2552 | | 2695756 | 2698245 | 2697580 | | |
| NMTN-07908 | SAV2552 | | 2695756 | 2698245 | 2697603 | | |
| NMTN-03159 | SAV2552 | | 2695756 | 2698245 | 2697850 | | |
| NMTN-09561 | SAV2552 | | 2695756 | 2698245 | 2698082 | | |
| NMTN-11169 | SAV2554 | rocA | 2699294 | 2700838 | 2699779 | − | 1-pyrroline-5-carboxylate |
| | | | | | | | dehydrogenase |
| NMTN-04868 | SAV2554 | rocA | 2699294 | 2700838 | 2699931 | | |
| NMTN-08467 | SAV2554 | rocA | 2699294 | 2700838 | 2699956 | | |
| NMTN-11464 | SAV2554 | rocA | 2699294 | 2700838 | 2700048 | | |
| NMTN-00458 | SAV2554 | rocA | 2699294 | 2700838 | 2700378 | | |
| NMTN-07409 | SAV2554 | rocA | 2699294 | 2700838 | 2700639 | | |
| NMTN-08445 | SAV2555 | | 2701028 | 2701627 | 2701025 | − | hypothetical protein |
| NMTN-08913 | SAV2555 | | 2701028 | 2701627 | 2701314 | | |
| NMTN-03601 | SAV2555 | | 2701028 | 2701627 | 2701367 | | |
| NMTN-07473 | SAV2555 | | 2701028 | 2701627 | 2701382 | | |
| NMTN-02145 | SAV2555 | | 2701028 | 2701627 | 2701444 | | |
| NMTN-04387 | SAV2557 | copA | 2702297 | 2704705 | 2702439 | + | copper-transporting ATPase |
| NMTN-06372 | SAV2557 | copA | 2702297 | 2704705 | 2702763 | | |
| NMTN-09344 | SAV2557 | copA | 2702297 | 2704705 | 2702856 | | |
| NMTN-04996 | SAV2557 | copA | 2702297 | 2704705 | 2703065 | | |
| NMTN-05541 | SAV2557 | copA | 2702297 | 2704705 | 2703227 | | |
| NMTN-11363 | SAV2557 | copA | 2702297 | 2704705 | 2703233 | | |
| NMTN-11079 | SAV2557 | copA | 2702297 | 2704705 | 2704038 | | |
| NMTN-12878 | SAV2557 | copA | 2702297 | 2704705 | 2704274 | | |
| NMTN-09427 | SAV2557 | copA | 2702297 | 2704705 | 2704392 | | |
| NMTN-11762 | SAV2558 | | 2705243 | 2705449 | 2705434 | + | hypothetical protein |
| NMTN-08473 | SAV2559 | | 2705540 | 2706538 | 2705734 | − | hypothetical protein |
| NMTN-12617 | SAV2560 | | 2706561 | 2707715 | 2706764 | − | hypothetical protein |
| NMTN-01831 | SAV2560 | | 2706561 | 2707715 | 2706765 | | |
| NMTN-01878 | SAV2560 | | 2706561 | 2707715 | 2706765 | | |
| NMTN-02271 | SAV2560 | | 2706561 | 2707715 | 2706905 | | |
| NMTN-06348 | SAV2560 | | 2706561 | 2707715 | 2707072 | | |
| NMTN-01477 | SAV2560 | | 2706561 | 2707715 | 2707400 | | |
| NMTN-13593 | SAV2560 | | 2706561 | 2707715 | 2707585 | | |
| NMTN-13594 | SAV2560 | | 2706561 | 2707715 | 2707585 | | |
| NMTN-00022 | SAV2561 | crtN | 2708143 | 2709651 | 2708287 | − | squalene synthase |
| NMTN-11447 | SAV2561 | crtN | 2708143 | 2709651 | 2708393 | | |
| NMTN-03594 | SAV2561 | crtN | 2708143 | 2709651 | 2709131 | | |
| NMTN-15226 | SAV2561 | crtN | 2708143 | 2709651 | 2709202 | | |
| NMTN-13713 | SAV2562 | crtM | 2709663 | 2710526 | 2709663 | − | squalene desaturase |
| NMTN-07303 | SAV2562 | crtM | 2709663 | 2710526 | 2709665 | | |
| NMTN-03760 | SAV2562 | crtM | 2709663 | 2710526 | 2709783 | | |
| NMTN-14521 | SAV2562 | crtM | 2709663 | 2710526 | 2709786 | | |
| NMTN-12847 | SAV2562 | crtM | 2709663 | 2710526 | 2709790 | | |
| NMTN-08294 | SAV2562 | crtM | 2709663 | 2710526 | 2709801 | | |
| NMTN-01557 | SAV2562 | crtM | 2709663 | 2710526 | 2709826 | | |
| NMTN-06949 | SAV2562 | crtM | 2709663 | 2710526 | 2709877 | | |
| NMTN-07682 | SAV2562 | crtM | 2709663 | 2710526 | 2710037 | | |
| NMTN-00663 | SAV2562 | crtM | 2709663 | 2710526 | 2710068 | | |
| NMTN-01593 | SAV2562 | crtM | 2709663 | 2710526 | 2710194 | | |
| NMTN-01602 | SAV2562 | crtM | 2709663 | 2710526 | 2710194 | | |
| NMTN-06581 | SAV2562 | crtM | 2709663 | 2710526 | 2710537 | | |
| NMTN-08865 | SAV2563 | | 2710563 | 2711690 | 2711070 | − | conserved hypothetical protein |
| NMTN-02946 | SAV2563 | | 2710563 | 2711690 | 2711210 | | |
| NMTN-12883 | SAV2564 | | 2711696 | 2713189 | 2711949 | − | hypothetical protein |
| NMTN-11414 | SAV2564 | | 2711696 | 2713189 | 2712018 | | |
| NMTN-14780 | SAV2564 | | 2711696 | 2713189 | 2712156 | | |
| NMTN-09114 | SAV2564 | | 2711696 | 2713189 | 2712342 | | |
| NMTN-14702 | SAV2564 | | 2711696 | 2713189 | 2712441 | | |
| NMTN-11654 | SAV2564 | | 2711696 | 2713189 | 2712809 | | |
| NMTN-08373 | SAV2565 | | 2713182 | 2713679 | 2713445 | − | hypothetical protein |
| NMTN-04150 | SAV2565 | | 2713182 | 2713679 | 2713659 | | |
| NMTN-05085 | SAV2566 | | 2713845 | 2714612 | 2714152 | − | hypothetical protein |
| NMTN-04670 | SAV2566 | | 2713845 | 2714612 | 2714325 | | |
| NMTN-00179 | SAV2566 | | 2713845 | 2714612 | 2714370 | | |
| NMTN-00516 | SAV2566 | | 2713845 | 2714612 | 2714370 | | |
| NMTN-06336 | SAV2566 | | 2713845 | 2714612 | 2714371 | | |
| NMTN-14402 | SAV2566 | | 2713845 | 2714612 | 2714394 | | |
| NMTN-03895 | SAV2566 | | 2713845 | 2714612 | 2714476 | | |
| NMTN-08500 | SAV2567 | | 2714973 | 2716784 | 2715119 | − | hypothetical protein |
| NMTN-10792 | SAV2567 | | 2714973 | 2716784 | 2715226 | | |
| NMTN-12964 | SAV2567 | | 2714973 | 2716784 | 2715306 | | |
| NMTN-12969 | SAV2567 | | 2714973 | 2716784 | 2715306 | | |
| NMTN-12970 | SAV2567 | | 2714973 | 2716784 | 2715306 | | |
| NMTN-12988 | SAV2567 | | 2714973 | 2716784 | 2715306 | | |
| NMTN-12994 | SAV2567 | | 2714973 | 2716784 | 2715306 | | |
| NMTN-13000 | SAV2567 | | 2714973 | 2716784 | 2715306 | | |
| NMTN-13012 | SAV2567 | | 2714973 | 2716784 | 2715306 | | |
| NMTN-13018 | SAV2567 | | 2714973 | 2716784 | 2715306 | | |
| NMTN-13024 | SAV2567 | | 2714973 | 2716784 | 2715306 | | |
| NMTN-13030 | SAV2567 | | 2714973 | 2716784 | 2715306 | | |
| NMTN-13031 | SAV2567 | | 2714973 | 2716784 | 2715306 | | |
| NMTN-09897 | SAV2567 | | 2714973 | 2716784 | 2715397 | | |
| NMTN-08343 | SAV2567 | | 2714973 | 2716784 | 2715665 | | |
| NMTN-07465 | SAV2567 | | 2714973 | 2716784 | 2715793 | | |
| NMTN-04546 | SAV2567 | | 2714973 | 2716784 | 2715839 | | |
| NMTN-12132 | SAV2567 | | 2714973 | 2716784 | 2715848 | | |
| NMTN-02562 | SAV2567 | | 2714973 | 2716784 | 2715853 | | |
| NMTN-03636 | SAV2567 | | 2714973 | 2716784 | 2715853 | | |
| NMTN-04076 | SAV2567 | | 2714973 | 2716784 | 2715872 | | |
| NMTN-01688 | SAV2567 | | 2714973 | 2716784 | 2715875 | | |
| NMTN-08515 | SAV2567 | | 2714973 | 2716784 | 2715978 | | |
| NMTN-14763 | SAV2567 | | 2714973 | 2716784 | 2716123 | | |
| NMTN-13728 | SAV2567 | | 2714973 | 2716784 | 2716286 | | |
| NMTN-07459 | SAV2567 | | 2714973 | 2716784 | 2716328 | | |
| NMTN-12078 | SAV2567 | | 2714973 | 2716784 | 2716380 | | |
| NMTN-13369 | SAV2567 | | 2714973 | 2716784 | 2716387 | | |
| NMTN-00792 | SAV2567 | | 2714973 | 2716784 | 2716421 | | |
| NMTN-02863 | SAV2567 | | 2714973 | 2716784 | 2716423 | | |
| NMTN-13285 | SAV2568 | | 2717297 | 2717785 | 2717325 | − | conserved hypothetical protein |
| NMTN-11346 | SAV2568 | | 2717297 | 2717785 | 2717341 | | |
| NMTN-02695 | SAV2568 | | 2717297 | 2717785 | 2717581 | | |
| NMTN-00237 | SAV2569 | isaA | 2718061 | 2718762 | 2718061 | − | immunodominant antigen A |
| NMTN-13112 | SAV2570 | | 2719370 | 2720425 | 2719477 | − | hypothetical protein |
| NMTN-06710 | SAV2570 | | 2719370 | 2720425 | 2719594 | | |
| NMTN-09324 | SAV2570 | | 2719370 | 2720425 | 2719668 | | |
| NMTN-13770 | SAV2570 | | 2719370 | 2720425 | 2719798 | | |
| NMTN-08644 | SAV2571 | | 2720835 | 2721404 | 2720846 | + | hypothetical protein |
| NMTN-14301 | SAV2571 | | 2720835 | 2721404 | 2721128 | | |
| NMTN-03781 | SAV2572 | | 2721782 | 2722102 | 2721825 | + | hypothetical protein |
| NMTN-03529 | SAV2572 | | 2721782 | 2722102 | 2721827 | | |
| NMTN-12347 | SAV2572 | | 2721782 | 2722102 | 2721949 | | |
| NMTN-12355 | SAV2572 | | 2721782 | 2722102 | 2721951 | | |
| NMTN-03508 | SAV2572 | | 2721782 | 2722102 | 2721963 | | |
| NMTN-05596 | SAV2572 | | 2721782 | 2722102 | 2722015 | | |
| NMTN-09298 | SAV2572 | | 2721782 | 2722102 | 2722015 | | |
| NMTN-11528 | SAV2572 | | 2721782 | 2722102 | 2722015 | | |
| NMTN-10619 | SAV2573 | | 2722322 | 2722552 | 2722448 | + | conserved hypothetical protein |
| NMTN-00838 | SAV2576 | | 2723248 | 2724069 | 2723529 | − | conserved hypothetical protein |
| NMTN-09414 | SAV2576 | | 2723248 | 2724069 | 2723753 | | |
| NMTN-04774 | SAV2576 | | 2723248 | 2724069 | 2723940 | | |
| NMTN-02527 | SAV2576 | | 2723248 | 2724069 | 2723942 | | |
| NMTN-07871 | SAV2576 | | 2723248 | 2724069 | 2723972 | | |
| NMTN-11841 | SAV2578 | | 2724519 | 2725076 | 2724627 | + | hypothetical protein |
| NMTN-03283 | SAV2578 | | 2724519 | 2725076 | 2724932 | | |
| NMTN-11432 | SAV2579 | | 2725069 | 2725773 | 2725159 | + | hypothetical protein |
| NMTN-14297 | SAV2579 | | 2725069 | 2725773 | 2725178 | | |
| NMTN-11570 | SAV2579 | | 2725069 | 2725773 | 2725223 | | |
| NMTN-12234 | SAV2579 | | 2725069 | 2725773 | 2725228 | | |
| NMTN-12596 | SAV2580 | | 2725871 | 2726881 | 2725902 | + | conserved hypothetical protein |
| NMTN-04358 | SAV2580 | | 2725871 | 2726881 | 2726834 | | |
| NMTN-14009 | SAV2581 | | 2726901 | 2727731 | 2727191 | + | conserevd hypothetical protein |
| NMTN-07148 | SAV2582 | | 2727878 | 2728768 | 2727899 | − | hypothetical protein |
| NMTN-07148 | SAV2582 | | 2727878 | 2728768 | 2727899 | | |
| NMTN-04499 | SAV2582 | | 2727878 | 2728768 | 2728468 | | |
| NMTN-05189 | SAV2582 | | 2727878 | 2728768 | 2728762 | | |
| NMTN-08001 | SAV2583 | | 2728865 | 2730205 | 2729090 | − | hypothetical protein |
| NMTN-11809 | SAV2583 | | 2728865 | 2730205 | 2729632 | | |
| NMTN-13519 | SAV2584 | | 2730199 | 2731305 | 2730353 | − | conserved hypothetical protein |
| NMTN-00657 | SAV2584 | | 2730199 | 2731305 | 2730395 | | |
| NMTN-06313 | SAV2584 | | 2730199 | 2731305 | 2730606 | | |
| NMTN-13422 | SAV2584 | | 2730199 | 2731305 | 2730722 | | |
| NMTN-07887 | SAV2584 | | 2730199 | 2731305 | 2731059 | | |
| NMTN-12457 | SAV2584 | | 2730199 | 2731305 | 2731076 | | |
| NMTN-02598 | SAV2584 | | 2730199 | 2731305 | 2731257 | | |
| NMTN-02997 | SAV2584 | | 2730199 | 2731305 | 2731292 | | |
| NMTN-00421 | SAV2585 | | 2731638 | 2731934 | 2731922 | − | conserved hypothetical protein |
| NMTN-10636 | SAV2587 | | 2732305 | 2732580 | 2732376 | − | hypothetical protein |
| NMTN-10654 | SAV2587 | | 2732305 | 2732580 | 2732376 | | |
| NMTN-10910 | SAV2587 | | 2732305 | 2732580 | 2732376 | | |
| NMTN-10922 | SAV2587 | | 2732305 | 2732580 | 2732376 | | |
| NMTN-10643 | SAV2587 | | 2732305 | 2732580 | 2732382 | | |
| NMTN-10939 | SAV2587 | | 2732305 | 2732580 | 2732399 | | |
| NMTN-04914 | SAV2588 | | 2732924 | 2733790 | 2733226 | − | conserved hypothetical protein |
| NMTN-04893 | SAV2591 | | 2735688 | 2736410 | 2736259 | − | conserved hypothetical protein |
| NMTN-12366 | SAV2591 | | 2735688 | 2736410 | 2736286 | | |
| NMTN-12473 | SAV2594 | | 2738024 | 2739706 | 2738057 | + | conserved hypothetical protein |
| NMTN-03919 | SAV2594 | | 2738024 | 2739706 | 2739296 | | |
| NMTN-03406 | SAV2594 | | 2738024 | 2739706 | 2739354 | | |
| NMTN-09691 | SAV2594 | | 2738024 | 2739706 | 2739470 | | |
| NMTN-02296 | SAV2597 | panD | 2741874 | 2742245 | 2741965 | − | aspartate 1-decarboxylase |
| NMTN-02521 | SAV2598 | panC | 2742259 | 2743110 | 2742276 | − | pantoate beta-alanine ligase |
| NMTN-15175 | SAV2598 | panC | 2742259 | 2743110 | 2742280 | | |
| NMTN-07451 | SAV2598 | panC | 2742259 | 2743110 | 2742423 | | |
| NMTN-09121 | SAV2598 | panC | 2742259 | 2743110 | 2742813 | | |
| NMTN-11785 | SAV2598 | panC | 2742259 | 2743110 | 2742813 | | |
| NMTN-13997 | SAV2599 | panB | 2743103 | 2743921 | 2743390 | − | 3-methyl-2-oxobutanoate |
| | | | | | | | hydroxymethyltransferase |
| NMTN-06644 | SAV2599 | panB | 2743103 | 2743921 | 2743753 | | |
| NMTN-06886 | SAV2600 | | 2743994 | 2744854 | 2744051 | + | hypothetical protein |
| NMTN-13807 | SAV2600 | | 2743994 | 2744854 | 2744241 | | |
| NMTN-10884 | SAV2600 | | 2743994 | 2744854 | 2744284 | | |
| NMTN-05452 | SAV2600 | | 2743994 | 2744854 | 2744403 | | |
| NMTN-06081 | SAV2600 | | 2743994 | 2744854 | 2744462 | | |
| NMTN-09112 | SAV2600 | | 2743994 | 2744854 | 2744487 | | |
| NMTN-00189 | SAV2600 | | 2743994 | 2744854 | 2744533 | | |
| NMTN-04810 | SAV2600 | | 2743994 | 2744854 | 2744597 | | |
| NMTN-12720 | SAV2602 | | 2745922 | 2746881 | 2746068 | − | L-lactate dehydrogenase |
| NMTN-07805 | SAV2602 | | 2745922 | 2746881 | 2746189 | | |
| NMTN-13035 | SAV2603 | | 2747567 | 2749015 | 2748068 | + | hypothetical protein |
| NMTN-14529 | SAV2603 | | 2747567 | 2749015 | 2748399 | | |
| NMTN-07788 | SAV2603 | | 2747567 | 2749015 | 2748419 | | |
| NMTN-13324 | SAV2603 | | 2747567 | 2749015 | 2748756 | | |
| NMTN-06796 | SAV2603 | | 2747567 | 2749015 | 2748793 | | |
| NMTN-09991 | SAV2604 | | 2749096 | 2750469 | 2749169 | − | hypothetical protein |
| NMTN-14466d | SAV2604 | | 2749096 | 2750469 | 2749250 | | |
| NMTN-14968 | SAV2604 | | 2749096 | 2750469 | 2749250 | | |
| NMTN-15091 | SAV2604 | | 2749096 | 2750469 | 2749250 | | |
| NMTN-00171 | SAV2604 | | 2749096 | 2750469 | 2749286 | | |
| NMTN-00265 | SAV2604 | | 2749096 | 2750469 | 2749286 | | |
| NMTN-13360 | SAV2604 | | 2749096 | 2750469 | 2749387 | | |
| NMTN-02730 | SAV2604 | | 2749096 | 2750469 | 2749391 | | |
| NMTN-11036 | SAV2604 | | 2749096 | 2750469 | 2749805 | | |
| NMTN-13720 | SAV2604 | | 2749096 | 2750469 | 2750192 | | |
| NMTN-04971 | SAV2605 | | 2750691 | 2751107 | 2750817 | + | hypothetical protein |
| NMTN-00109 | SAV2605 | | 2750691 | 2751107 | 2750939 | | |
| NMTN-08555 | SAV2606 | | 2751230 | 2752120 | 2751484 | + | fructose-bisphosphate aldolase homolog |
| NMTN-08558 | SAV2606 | | 2751230 | 2752120 | 2751484 | | |
| NMTN-12975 | SAV2606 | | 2751230 | 2752120 | 2751485 | | |
| NMTN-08544 | SAV2606 | | 2751230 | 2752120 | 2751496 | | |
| NMTN-08556 | SAV2606 | | 2751230 | 2752120 | 2751498 | | |
| NMTN-00773 | SAV2607 | mqo2 | 2752314 | 2753810 | 2752712 | − | malate/quinone oxidoreductase |
| NMTN-11441 | SAV2608 | | 2754187 | 2754435 | 2754251 | + | hypothetical protein |
| NMTN-07242 | SAV2608 | | 2754187 | 2754435 | 2754260 | | |
| NMTN-11969 | SAV2608 | | 2754187 | 2754435 | 2754346 | | |
| NMTN-04821 | SAV2609 | | 2754495 | 2756114 | 2754730 | − | acetate-CoA ligase |
| NMTN-14556 | SAV2609 | | 2754495 | 2756114 | 2755876 | | |
| NMTN-10562 | SAV2610 | | 2756287 | 2756730 | 2756573 | − | conserved hypothetical protein |
| NMTN-08980 | SAV2610 | | 2756287 | 2756730 | 2756579 | | |
| NMTN-01537 | SAV2612 | betA | 2757500 | 2759209 | 2757572 | − | choline dehydrogenase |
| NMTN-06805 | SAV2612 | betA | 2757500 | 2759209 | 2758094 | | |
| NMTN-09266 | SAV2612 | betA | 2757500 | 2759209 | 2758298 | | |
| NMTN-08137 | SAV2612 | betA | 2757500 | 2759209 | 2758405 | | |
| NMTN-04344 | SAV2612 | betA | 2757500 | 2759209 | 2758468 | | |
| NMTN-07263 | SAV2612 | betA | 2757500 | 2759209 | 2758468 | | |
| NMTN-11071 | SAV2612 | betA | 2757500 | 2759209 | 2758680 | | |
| NMTN-04143 | SAV2613 | gbsA | 2759470 | 2760960 | 2759536 | − | glycine betaine aldehyde |
| | | | | | | | dehydrogenase |
| NMTN-02851 | SAV2613 | gbsA | 2759470 | 2760960 | 2759716 | | |
| NMTN-02132 | SAV2613 | gbsA | 2759470 | 2760960 | 2759766 | | |
| NMTN-05070 | SAV2613 | gbsA | 2759470 | 2760960 | 2759948 | | |
| NMTN-06306 | SAV2613 | gbsA | 2759470 | 2760960 | 2760516 | | |
| NMTN-04535 | SAV2613 | gbsA | 2759470 | 2760960 | 2760638 | | |
| NMTN-06718 | SAV2614 | | 2761210 | 2761776 | 2761327 | + | conserved hypothetical protein |
| NMTN-01962 | SAV2615 | cudT | 2761947 | 2763569 | 2762451 | − | choline transporter |
| NMTN-04088 | SAV2615 | cudT | 2761947 | 2763569 | 2762577 | | |
| NMTN-04054 | SAV2615 | cudT | 2761947 | 2763569 | 2762595 | | |
| NMTN-06875 | SAV2615 | cudT | 2761947 | 2763569 | 2762900 | | |
| NMTN-11177 | SAV2615 | cudT | 2761947 | 2763569 | 2763069 | | |
| NMTN-05071 | SAV2615 | cudT | 2761947 | 2763569 | 2763126 | | |
| NMTN-02034 | SAV2615 | cudT | 2761947 | 2763569 | 2763312 | | |
| NMTN-12512 | SAV2616 | | 2764088 | 2764624 | 2764144 | − | hypothetical protein |
| NMTN-04459 | SAV2616 | | 2764088 | 2764624 | 2764234 | | |
| NMTN-04385 | SAV2617 | nrdD | 2764621 | 2766471 | 2765009 | − | anaerobic ribonucleoside-triphosphate |
| | | | | | | | reductase |
| NMTN-07822 | SAV2617 | nrdD | 2764621 | 2766471 | 2765015 | | |
| NMTN-11600 | SAV2617 | nrdD | 2764621 | 2766471 | 2765354 | | |
| NMTN-09460 | SAV2617 | nrdD | 2764621 | 2766471 | 2765713 | | |
| NMTN-12940 | SAV2617 | nrdD | 2764621 | 2766471 | 2766171 | | |
| NMTN-03796 | SAV2618 | | 2766661 | 2768031 | 2766739 | − | hypothetical protein |
| NMTN-13806 | SAV2618 | | 2766661 | 2768031 | 2766746 | | |
| NMTN-12389 | SAV2618 | | 2766661 | 2768031 | 2766789 | | |
| NMTN-14506 | SAV2618 | | 2766661 | 2768031 | 2766855 | | |
| NMTN-03822 | SAV2618 | | 2766661 | 2768031 | 2767182 | | |
| NMTN-02850 | SAV2618 | | 2766661 | 2768031 | 2767353 | | |
| NMTN-13493 | SAV2618 | | 2766661 | 2768031 | 2767353 | | |
| NMTN-12152 | SAV2618 | | 2766661 | 2768031 | 2767356 | | |
| NMTN-02491 | SAV2618 | | 2766661 | 2768031 | 2767622 | | |
| NMTN-12676 | SAV2618 | | 2766661 | 2768031 | 2767639 | | |
| NMTN-07957 | SAV2618 | | 2766661 | 2768031 | 2767704 | | |
| NMTN-07118 | SAV2619 | | 2768524 | 2769129 | 2768661 | − | hypothetical protein |
| NMTN-12167 | SAV2619 | | 2768524 | 2769129 | 2768765 | | |
| NMTN-08426 | SAV2619 | | 2768524 | 2769129 | 2768971 | | |
| NMTN-03468 | SAV2620 | | 2769192 | 2771072 | 2769410 | − | sulfite reductase flavoprotein |
| NMTN-11305 | SAV2620 | | 2769192 | 2771072 | 2769689 | | |
| NMTN-12193 | SAV2620 | | 2769192 | 2771072 | 2769786 | | |
| NMTN-07081 | SAV2620 | | 2769192 | 2771072 | 2770030 | | |
| NMTN-10714 | SAV2620 | | 2769192 | 2771072 | 2770051 | | |
| NMTN-11923 | SAV2620 | | 2769192 | 2771072 | 2770225 | | |
| NMTN-00268 | SAV2620 | | 2769192 | 2771072 | 2770457 | | |
| NMTN-01461 | SAV2620 | | 2769192 | 2771072 | 2770477 | | |
| NMTN-02085 | SAV2620 | | 2769192 | 2771072 | 2770698 | | |
| NMTN-13059 | SAV2620 | | 2769192 | 2771072 | 2770854 | | |
| NMTN-12700 | SAV2621 | | 2771466 | 2771963 | 2771495 | − | hypothetical protein |
| NMTN-03913 | SAV2621 | | 2771466 | 2771963 | 2771507 | | |
| NMTN-02612 | SAV2621 | | 2771466 | 2771963 | 2771630 | | |
| NMTN-13290 | SAV2621 | | 2771466 | 2771963 | 2771803 | | |
| NMTN-10657 | SAV2621 | | 2771466 | 2771963 | 2771851 | | |
| NMTN-14036d | SAV2621 | | 2771466 | 2771963 | 2771851 | | |
| NMTN-03424 | SAV2622 | | 2773387 | 2775387 | 2773453 | − | hypothetical protein |
| NMTN-04001 | SAV2622 | | 2773387 | 2775387 | 2773476 | | |
| NMTN-09285 | SAV2622 | | 2773387 | 2775387 | 2773483 | | |
| NMTN-10873 | SAV2622 | | 2773387 | 2775387 | 2773499 | | |
| NMTN-03533 | SAV2622 | | 2773387 | 2775387 | 2773767 | | |
| NMTN-03977 | SAV2622 | | 2773387 | 2775387 | 2773936 | | |
| NMTN-11160 | SAV2622 | | 2773387 | 2775387 | 2774394 | | |
| NMTN-14619 | SAV2622 | | 2773387 | 2775387 | 2774621 | | |
| NMTN-06543 | SAV2622 | | 2773387 | 2775387 | 2775249 | | |
| NMTN-13515 | SAV2623 | | 2775384 | 2776139 | 2775482 | − | hypothetical protein |
| NMTN-01691 | SAV2623 | | 2775384 | 2776139 | 2775647 | | |
| NMTN-05569 | SAV2623 | | 2775384 | 2776139 | 2775769 | | |
| NMTN-03689 | SAV2623 | | 2775384 | 2776139 | 2775785 | | |
| NMTN-03452 | SAV2623 | | 2775384 | 2776139 | 2775799 | | |
| NMTN-05069 | SAV2624 | | 2776247 | 2777134 | 2776740 | − | hypothetical protein |
| NMTN-06186 | SAV2624 | | 2776247 | 2777134 | 2776984 | | |
| NMTN-05304 | SAV2625 | | 2777145 | 2777810 | 2777457 | − | hypothetical protein |
| NMTN-12784 | SAV2625 | | 2777145 | 2777810 | 2777660 | | |
| NMTN-02121 | SAV2627 | phoB | 2778234 | 2779658 | 2779030 | + | alkaline phosphatase III precursor |
| NMTN-15181 | SAV2627 | phoB | 2778234 | 2779658 | 2779089 | | |
| NMTN-04781 | SAV2627 | phoB | 2778234 | 2779658 | 2779195 | | |
| NMTN-13911d | SAV2627 | phoB | 2778234 | 2779658 | 2779195 | | |
| NMTN-09454 | SAV2627 | phoB | 2778234 | 2779658 | 2779309 | | |
| NMTN-13140 | SAV2629 | | 2780641 | 2781402 | 2781185 | − | hypothetical protein |
| NMTN-08684 | SAV2629 | | 2780641 | 2781402 | 2781195 | | |
| NMTN-02802 | SAV2629 | | 2780641 | 2781402 | 2781227 | | |
| NMTN-06861 | SAV2629 | | 2780641 | 2781402 | 2781238 | | |
| NMTN-14705 | SAV2629 | | 2780641 | 2781402 | 2781238 | | |
| NMTN-13125 | SAV2630 | clfB | 2781518 | 2784151 | 2781516 | | |
| NMTN-13153 | SAV2630 | clfB | 2781518 | 2784151 | 2781516 | | |
| NMTN-07323 | SAV2630 | clfB | 2781518 | 2784151 | 2782704 | − | Clumping factor B |
| NMTN-03615 | SAV2630 | clfB | 2781518 | 2784151 | 2782814 | | |
| NMTN-02818 | SAV2630 | clfB | 2781518 | 2784151 | 2782956 | | |
| NMTN-11940 | SAV2630 | clfB | 2781518 | 2784151 | 2783056 | | |
| NMTN-11936 | SAV2630 | clfB | 2781518 | 2784151 | 2783081 | | |
| NMTN-00642 | SAV2630 | clfB | 2781518 | 2784151 | 2783132 | | |
| NMTN-03129 | SAV2630 | clfB | 2781518 | 2784151 | 2783211 | | |
| NMTN-12227 | SAV2630 | clfB | 2781518 | 2784151 | 2783212 | | |
| NMTN-08433 | SAV2630 | clfB | 2781518 | 2784151 | 2783298 | | |
| NMTN-00826 | SAV2630 | clfB | 2781518 | 2784151 | 2783316 | | |
| NMTN-15231 | SAV2630 | clfB | 2781518 | 2784151 | 2783557 | | |
| NMTN-07441 | SAV2630 | clfB | 2781518 | 2784151 | 2783954 | | |
| NMTN-05159 | SAV2631 | | 2784501 | 2785205 | 2784971 | − | hypothetical protein |
| NMTN-13909 | SAV2631 | | 2784501 | 2785205 | 2785060 | | |
| NMTN-07559 | SAV2631 | | 2784501 | 2785205 | 2785120 | | |
| NMTN-10668 | SAV2632 | arcC | 2785304 | 2786272 | 2785332 | − | carbamate kinase |
| NMTN-04271 | SAV2632 | arcC | 2785304 | 2786272 | 2785550 | | |
| NMTN-07288 | SAV2633 | arcD | 2786262 | 2787692 | 2786315 | − | arginine/oirnithine antiporter |
| NMTN-00539 | SAV2633 | arcD | 2786262 | 2787692 | 2786433 | | |
| NMTN-12496 | SAV2633 | arcD | 2786262 | 2787692 | 2786456 | | |
| NMTN-03994 | SAV2633 | arcD | 2786262 | 2787692 | 2786458 | | |
| NMTN-04673 | SAV2633 | arcD | 2786262 | 2787692 | 2786503 | | |
| NMTN-05038 | SAV2633 | arcD | 2786262 | 2787692 | 2786927 | | |
| NMTN-02118 | SAV2633 | arcD | 2786262 | 2787692 | 2786940 | | |
| NMTN-00746 | SAV2634 | arcB | 2787783 | 2788793 | 2788093 | − | ornithine transcarbamoylase |
| NMTN-03256 | SAV2634 | arcB | 2787783 | 2788793 | 2788188 | | |
| NMTN-13890 | SAV2634 | arcB | 2787783 | 2788793 | 2788319 | | |
| NMTN-01608 | SAV2634 | arcB | 2787783 | 2788793 | 2788498 | | |
| NMTN-04053 | SAV2635 | arcA | 2788826 | 2790061 | 2788948 | − | arginine deiminase |
| NMTN-03618 | SAV2635 | arcA | 2788826 | 2790061 | 2788949 | | |
| NMTN-05244 | SAV2635 | arcA | 2788826 | 2790061 | 2788986 | | |
| NMTN-14719d | SAV2635 | arcA | 2788826 | 2790061 | 2788998 | | |
| NMTN-02276 | SAV2635 | arcA | 2788826 | 2790061 | 2789164 | | |
| NMTN-12461 | SAV2635 | arcA | 2788826 | 2790061 | 2789227 | | |
| NMTN-07482 | SAV2635 | arcA | 2788826 | 2790061 | 2789246 | | |
| NMTN-10660 | SAV2635 | arcA | 2788826 | 2790061 | 2789358 | | |
| NMTN-04557 | SAV2635 | arcA | 2788826 | 2790061 | 2789361 | | |
| NMTN-13137 | SAV2635 | arcA | 2788826 | 2790061 | 2789628 | | |
| NMTN-11542d | SAV2635 | arcA | 2788826 | 2790061 | 2789858 | | |
| NMTN-06838 | SAV2635 | arcA | 2788826 | 2790061 | 2790016 | | |
| NMTN-13057d | SAV2636 | | 2790408 | 2790857 | 2790506 | − | hypothetical protein |
| NMTN-11761 | SAV2636 | | 2790408 | 2790857 | 2790576 | | |
| NMTN-14597 | SAV2636 | | 2790408 | 2790857 | 2790576 | | |
| NMTN-00400 | SAV2636 | | 2790408 | 2790857 | 2790618 | | |
| NMTN-12533 | SAV2636 | | 2790408 | 2790857 | 2790658 | | |
| NMTN-12752 | SAV2636 | | 2790408 | 2790857 | 2790748 | | |
| NMTN-09030 | SAV2637 | aur | 2791224 | 2792753 | 2792521 | − | zinc metalloproteinase aureolysin |
| NMTN-08682 | SAV2638 | isaB | 2793177 | 2793704 | 2793212 | − | immunodominant antigen B |
| NMTN-07630 | SAV2639 | | 2793962 | 2794420 | 2794006 | + | conserved hypothetical protein |
| NMTN-13671 | SAV2639 | | 2793962 | 2794420 | 2794287 | | |
| NMTN-11257 | SAV2640 | | 2794660 | 2796534 | 2794724 | + | hypothetical protein |
| NMTN-12185 | SAV2640 | | 2794660 | 2796534 | 2795247 | | |
| NMTN-05231 | SAV2640 | | 2794660 | 2796534 | 2795540 | | |
| NMTN-04751 | SAV2640 | | 2794660 | 2796534 | 2795731 | | |
| NMTN-05700 | SAV2640 | | 2794660 | 2796534 | 2795947 | | |
| NMTN-00195 | SAV2640 | | 2794660 | 2796534 | 2796034 | | |
| NMTN-00389 | SAV2640 | | 2794660 | 2796534 | 2796034 | | |
| NMTN-04988 | SAV2640 | | 2794660 | 2796534 | 2796136 | | |
| NMTN-07828 | SAV2640 | | 2794660 | 2796534 | 2796487 | | |
| NMTN-00382 | SAV2641 | | 2796610 | 2798562 | 2796656 | + | fructose phosphotransferase system |
| | | | | | | | enzyme fruA homolog |
| NMTN-02784 | SAV2641 | | 2796610 | 2798562 | 2796657 | | |
| NMTN-13387 | SAV2641 | | 2796610 | 2798562 | 2796658 | | |
| NMTN-00588 | SAV2641 | | 2796610 | 2798562 | 2796660 | | |
| NMTN-00597 | SAV2641 | | 2796610 | 2798562 | 2796660 | | |
| NMTN-01473 | SAV2641 | | 2796610 | 2798562 | 2796660 | | |
| NMTN-12783d | SAV2641 | | 2796610 | 2798562 | 2796660 | | |
| NMTN-13358 | SAV2641 | | 2796610 | 2798562 | 2796660 | | |
| NMTN-13366 | SAV2641 | | 2796610 | 2798562 | 2796660 | | |
| NMTN-13383 | SAV2641 | | 2796610 | 2798562 | 2796660 | | |
| NMTN-13384 | SAV2641 | | 2796610 | 2798562 | 2796660 | | |
| NMTN-13418 | SAV2641 | | 2796610 | 2798562 | 2796660 | | |
| NMTN-13429 | SAV2641 | | 2796610 | 2798562 | 2796660 | | |
| NMTN-13431 | SAV2641 | | 2796610 | 2798562 | 2796660 | | |
| NMTN-05456 | SAV2641 | | 2796610 | 2798562 | 2797695 | | |
| NMTN-05040 | SAV2641 | | 2796610 | 2798562 | 2798288 | | |
| NMTN-08302 | SAV2641 | | 2796610 | 2798562 | 2798548 | | |
| NMTN-07093 | SAV2642 | pmi | 2798576 | 2799514 | 2798642 | + | mannose-6-phosphate isomerase |
| NMTN-07094 | SAV2642 | pmi | 2798576 | 2799514 | 2798706 | | |
| NMTN-12815 | SAV2642 | pmi | 2798576 | 2799514 | 2798728 | | |
| NMTN-04717 | SAV2642 | pmi | 2798576 | 2799514 | 2799358 | | |
| NMTN-06343 | SAV2643 | | 2799626 | 2802607 | 2799954 | − | hypothetical protein |
| NMTN-08272 | SAV2643 | | 2799626 | 2802607 | 2800203 | | |
| NMTN-11406 | SAV2643 | | 2799626 | 2802607 | 2802003 | | |
| NMTN-02659 | SAV2644 | | 2802818 | 2804677 | 2803509 | + | hypothetical protein |
| NMTN-14542 | SAV2644 | | 2802818 | 2804677 | 2803731 | | |
| NMTN-12767 | SAV2644 | | 2802818 | 2804677 | 2804324 | | |
| NMTN-07147 | SAV2644 | | 2802818 | 2804677 | 2804543 | | |
| NMTN-10479 | SAV2644 | | 2802818 | 2804677 | 2804625 | | |
| NMTN-05462 | SAV2645 | | 2804942 | 2805502 | 2805063 | − | hypothetical protein |
| NMTN-04073 | SAV2645 | | 2804942 | 2805502 | 2805068 | | |
| NMTN-00505 | SAV2646 | | 2805671 | 2807578 | 2805693 | − | conserved hypothetical protein |
| NMTN-12751 | SAV2646 | | 2805671 | 2807578 | 2805796 | | |
| NMTN-03078 | SAV2646 | | 2805671 | 2807578 | 2805931 | | |
| NMTN-09723 | SAV2646 | | 2805671 | 2807578 | 2806013 | | |
| NMTN-11485 | SAV2646 | | 2805671 | 2807578 | 2806581 | | |
| NMTN-10751 | SAV2646 | | 2805671 | 2807578 | 2806602 | | |
| NMTN-07999 | SAV2646 | | 2805671 | 2807578 | 2807267 | | |
| NMTN-02604 | SAV2646 | | 2805671 | 2807578 | 2807583 | | |
| NMTN-02354 | SAV2647 | | 2807819 | 2809177 | 2808116 | − | hypothetical protein |
| NMTN-12096 | SAV2647 | | 2807819 | 2809177 | 2808921 | | |
| NMTN-01876 | SAV2647 | | 2807819 | 2809177 | 2808973 | | |
| NMTN-12898 | SAV2647 | | 2807819 | 2809177 | 2809113 | | |
| NMTN-03626 | SAV2647 | | 2807819 | 2809177 | 2809133 | | |
| NMTN-03013 | SAV2648 | | 2809170 | 2810678 | 2809426 | − | hypothetical protein |
| NMTN-11879 | SAV2648 | | 2809170 | 2810678 | 2809528 | | |
| NMTN-13433 | SAV2648 | | 2809170 | 2810678 | 2809779 | | |
| NMTN-08657 | SAV2648 | | 2809170 | 2810678 | 2809899 | | |
| NMTN-03692 | SAV2648 | | 2809170 | 2810678 | 2810098 | | |
| NMTN-03105 | SAV2648 | | 2809170 | 2810678 | 2810123 | | |
| NMTN-13682 | SAV2648 | | 2809170 | 2810678 | 2810193 | | |
| NMTN-06262 | SAV2648 | | 2809170 | 2810678 | 2810248 | | |
| NMTN-00083 | SAV2648 | | 2809170 | 2810678 | 2810498 | | |
| NMTN-07965 | SAV2649 | | 2810696 | 2813086 | 2810739 | − | preprotein translocase secA homolog |
| NMTN-15186 | SAV2649 | | 2810696 | 2813086 | 2811202 | | |
| NMTN-06606 | SAV2649 | | 2810696 | 2813086 | 2811385 | | |
| NMTN-06536 | SAV2649 | | 2810696 | 2813086 | 2811395 | | |
| NMTN-07764 | SAV2649 | | 2810696 | 2813086 | 2811465 | | |
| NMTN-12755 | SAV2649 | | 2810696 | 2813086 | 2811537 | | |
| NMTN-00063 | SAV2649 | | 2810696 | 2813086 | 2811724 | | |
| NMTN-11870 | SAV2649 | | 2810696 | 2813086 | 2812243 | | |
| NMTN-06770 | SAV2649 | | 2810696 | 2813086 | 2812655 | | |
| NMTN-12662 | SAV2649 | | 2810696 | 2813086 | 2812865 | | |
| NMTN-04051 | SAV2649 | | 2810696 | 2813086 | 2812918 | | |
| NMTN-03906 | SAV2650 | | 2813076 | 2814116 | 2813317 | − | hypothetical protein |
| NMTN-06809 | SAV2650 | | 2813076 | 2814116 | 2813348 | | |
| NMTN-11845 | SAV2650 | | 2813076 | 2814116 | 2813645 | | |
| NMTN-00878 | SAV2650 | | 2813076 | 2814116 | 2813986 | | |
| NMTN-00273 | SAV2651 | | 2814013 | 2815581 | 2814119 | − | hypothetical protein |
| NMTN-11289 | SAV2651 | | 2814013 | 2815581 | 2814177 | | |
| NMTN-02497 | SAV2651 | | 2814013 | 2815581 | 2814180 | | |
| NMTN-15303 | SAV2651 | | 2814013 | 2815581 | 2814214 | | |
| NMTN-05152 | SAV2651 | | 2814013 | 2815581 | 2814220 | | |
| NMTN-04967 | SAV2651 | | 2814013 | 2815581 | 2814221 | | |
| NMTN-08126 | SAV2651 | | 2814013 | 2815581 | 2814342 | | |
| NMTN-15204 | SAV2651 | | 2814013 | 2815581 | 2814476 | | |
| NMTN-11731 | SAV2651 | | 2814013 | 2815581 | 2814824 | | |
| NMTN-02762 | SAV2651 | | 2814013 | 2815581 | 2814828 | | |
| NMTN-09267 | SAV2652 | | 2815571 | 2817124 | 2815629 | − | hypothetical protein |
| NMTN-04305 | SAV2652 | | 2815571 | 2817124 | 2816152 | | |
| NMTN-04313 | SAV2652 | | 2815571 | 2817124 | 2816152 | | |
| NMTN-11124 | SAV2652 | | 2815571 | 2817124 | 2816152 | | |
| NMTN-09398 | SAV2652 | | 2815571 | 2817124 | 2816158 | | |
| NMTN-08021 | SAV2652 | | 2815571 | 2817124 | 2816216 | | |
| NMTN-11181 | SAV2652 | | 2815571 | 2817124 | 2816296 | | |
| NMTN-02681 | SAV2652 | | 2815571 | 2817124 | 2816321 | | |
| NMTN-03558 | SAV2652 | | 2815571 | 2817124 | 2816920 | | |
| NMTN-09790 | SAV2653 | | 2817135 | 2818346 | 2817261 | − | hypothetical protein |
| NMTN-10579 | SAV2653 | | 2817135 | 2818346 | 2817314 | | |
| NMTN-12290 | SAV2653 | | 2817135 | 2818346 | 2817317 | | |
| NMTN-02810 | SAV2653 | | 2817135 | 2818346 | 2817359 | | |
| NMTN-08822 | SAV2653 | | 2817135 | 2818346 | 2817548 | | |
| NMTN-08053 | SAV2653 | | 2817135 | 2818346 | 2817560 | | |
| NMTN-07973 | SAV2653 | | 2817135 | 2818346 | 2817561 | | |
| NMTN-01728 | SAV2653 | | 2817135 | 2818346 | 2817671 | | |
| NMTN-01583 | SAV2653 | | 2817135 | 2818346 | 2817671 | | |
| NMTN-10706 | SAV2653 | | 2817135 | 2818346 | 2817702 | | |
| NMTN-13893 | SAV2653 | | 2817135 | 2818346 | 2817702 | | |
| NMTN-06395 | SAV2653 | | 2817135 | 2818346 | 2817705 | | |
| NMTN-07279 | SAV2653 | | 2817135 | 2818346 | 2817800 | | |
| NMTN-11024 | SAV2653 | | 2817135 | 2818346 | 2817803 | | |
| NMTN-07537 | SAV2653 | | 2817135 | 2818346 | 2817878 | | |
| NMTN-11118 | SAV2653 | | 2817135 | 2818346 | 2817944 | | |
| NMTN-03661 | SAV2654 | | 2818476 | 2825291 | 2818846 | − | hypothetical protein |
| NMTN-13337d | SAV2654 | | 2818476 | 2825291 | 2819383 | | |
| NMTN-13342 | SAV2654 | | 2818476 | 2825291 | 2819987 | | |
| NMTN-00264 | SAV2654 | | 2818476 | 2825291 | 2820277 | | |
| NMTN-09452 | SAV2654 | | 2818476 | 2825291 | 2820352 | | |
| NMTN-12564 | SAV2654 | | 2818476 | 2825291 | 2820352 | | |
| NMTN-11529 | SAV2654 | | 2818476 | 2825291 | 2822530 | | |
| NMTN-04676 | SAV2654 | | 2818476 | 2825291 | 2822536 | | |
| NMTN-02011 | SAV2654 | | 2818476 | 2825291 | 2823121 | | |
| NMTN-08225 | SAV2654 | | 2818476 | 2825291 | 2823198 | | |
| NMTN-08662 | SAV2654 | | 2818476 | 2825291 | 2823934 | | |
| NMTN-11137 | SAV2654 | | 2818476 | 2825291 | 2824069 | | |
| NMTN-11967 | SAV2654 | | 2818476 | 2825291 | 2824072 | | |
| NMTN-14518 | SAV2654 | | 2818476 | 2825291 | 2824483 | | |
| NMTN-05415 | SAV2654 | | 2818476 | 2825291 | 2824485 | | |
| NMTN-04712 | SAV2654 | | 2818476 | 2825291 | 2824739 | | |
| NMTN-12370 | SAV2654 | | 2818476 | 2825291 | 2824966 | | |
| NMTN-12578 | SAV2654 | | 2818476 | 2825291 | 2825236 | | |
| NMTN-06330 | SAV2655 | | 2825835 | 2826527 | 2825876 | − | conserved hypothetical protein |
| NMTN-11230 | SAV2658 | | 2827664 | 2828023 | 2827789 | + | hypothetical protein |
| NMTN-11724 | SAV2659 | | 2828108 | 2828719 | 2828635 | − | conserved hypothetical protein |
| NMTN-04485 | SAV2660 | | 2828798 | 2829274 | 2828829 | − | hypothetical protein |
| NMTN-03481 | SAV2660 | | 2828798 | 2829274 | 2828838 | | |
| NMTN-07977 | SAV2660 | | 2828798 | 2829274 | 2828839 | | |
| NMTN-06434 | SAV2660 | | 2828798 | 2829274 | 2828841 | | |
| NMTN-04057 | SAV2660 | | 2828798 | 2829274 | 2828897 | | |
| NMTN-11474 | SAV2660 | | 2828798 | 2829274 | 2828997 | | |
| NMTN-02386 | SAV2660 | | 2828798 | 2829274 | 2829137 | | |
| NMTN-02687 | SAV2661 | | 2829277 | 2829777 | 2829317 | | |
| NMTN-14612 | SAV2661 | | 2829277 | 2829777 | 2829526 | − | conserved hypothetical protein |
| NMTN-03156 | SAV2661 | | 2829277 | 2829777 | 2829647 | | |
| NMTN-07326 | SAV2662 | capC | 2830012 | 2830779 | 2830372 | − | capsular polysaccharide biosynthesis |
| NMTN-12920 | SAV2662 | capC | 2830012 | 2830779 | 2830437 | | |
| NMTN-11295 | SAV2662 | capC | 2830012 | 2830779 | 2830499 | | |
| NMTN-04689 | SAV2662 | capC | 2830012 | 2830779 | 2830509 | | |
| NMTN-02030 | SAV2663 | capB | 2830776 | 2831468 | 2830850 | − | capsular polysaccharide biosynthesis |
| NMTN-08394 | SAV2663 | capB | 2830776 | 2831468 | 2830899 | | |
| NMTN-11334 | SAV2663 | capB | 2830776 | 2831468 | 2831460 | | |
| NMTN-12143 | SAV2664 | capA | 2831485 | 2832147 | 2832019 | − | capsular polysaccharide biosynthesis |
| NMTN-11052c | SAV2665 | icaR | 2832996 | 2833556 | 2833178 | − | ica operon transcriptional regulator |
| NMTN-11044 | SAV2665 | icaR | 2832996 | 2833556 | 2833193 | | |
| NMTN-11831 | SAV2665 | icaR | 2832996 | 2833556 | 2833214 | | |
| NMTN-09269 | SAV2665 | icaR | 2832996 | 2833556 | 2833446 | | |
| NMTN-04722 | SAV2666 | icaA | 2833720 | 2834958 | 2833794 | + | intercellular adhesion protein A |
| NMTN-14410 | SAV2666 | icaA | 2833720 | 2834958 | 2833797 | | |
| NMTN-09625 | SAV2666 | icaA | 2833720 | 2834958 | 2833856 | | |
| NMTN-09758 | SAV2666 | icaA | 2833720 | 2834958 | 2833856 | | |
| NMTN-09770 | SAV2666 | icaA | 2833720 | 2834958 | 2833856 | | |
| NMTN-09806 | SAV2666 | icaA | 2833720 | 2834958 | 2833856 | | |
| NMTN-09841 | SAV2666 | icaA | 2833720 | 2834958 | 2833856 | | |
| NMTN-11465 | SAV2666 | icaA | 2833720 | 2834958 | 2834106 | | |
| NMTN-12499 | SAV2666 | icaA | 2833720 | 2834958 | 2834636 | | |
| NMTN-04780 | SAV2666 | icaA | 2833720 | 2834958 | 2834642 | | |
| NMTN-11914 | SAV2666 | icaA | 2833720 | 2834958 | 2834702 | | |
| NMTN-08875 | SAV2666 | icaA | 2833720 | 2834958 | 2834722 | | |
| NMTN-09217 | SAV2666 | icaA | 2833720 | 2834958 | 2834722 | | |
| NMTN-11413 | SAV2666 | icaA | 2833720 | 2834958 | 2834866 | | |
| NMTN-06087 | SAV2667 | icaD | 2834922 | 2835227 | 2835055 | + | intercellular adhesion protein D |
| NMTN-11335 | SAV2667 | icaD | 2834922 | 2835227 | 2835057 | | |
| NMTN-01598 | SAV2667 | icaD | 2834922 | 2835227 | 2835058 | | |
| NMTN-03146 | SAV2668 | icaB | 2835224 | 2836096 | 2835342 | + | intercellular adhesion protein B |
| NMTN-14557d | SAV2669 | icaC | 2836083 | 2837135 | 2836128 | + | intercellular adhesion protein C |
| NMTN-05081 | SAV2669 | icaC | 2836083 | 2837135 | 2836763 | | |
| NMTN-06051 | SAV2669 | icaC | 2836083 | 2837135 | 2837047 | | |
| NMTN-12308 | SAV2669 | icaC | 2836083 | 2837135 | 2837047 | | |
| NMTN-00266 | SAV2669 | icaC | 2836083 | 2837135 | 2837113 | | |
| NMTN-00325 | SAV2669 | icaC | 2836083 | 2837135 | 2837113 | | |
| NMTN-12300 | SAV2669 | icaC | 2836083 | 2837135 | 2837130 | | |
| NMTN-04872 | SAV2671 | lip | 2837470 | 2839515 | 2839112 | − | triacylglycerol lipase precursor |
| NMTN-12464 | SAV2672 | hisI | 2840605 | 2841237 | 2840858 | − | histidine biosynthesis bifunctional |
| | | | | | | | protein HisIE |
| NMTN-04758 | SAV2672 | hisI | 2840605 | 2841237 | 2841047 | | |
| NMTN-12061d | SAV2673 | hisF | 2841234 | 2841992 | 2841469 | − | cyclase-like protein hisF |
| NMTN-03963 | SAV2673 | hisF | 2841234 | 2841992 | 2841562 | | |
| NMTN-04869 | SAV2674 | | 2841989 | 2842693 | 2842191 | − | hypothetical protein |
| NMTN-03892 | SAV2674 | | 2841989 | 2842693 | 2842195 | | |
| NMTN-09374 | SAV2674 | | 2841989 | 2842693 | 2842216 | | |
| NMTN-09380 | SAV2674 | | 2841989 | 2842693 | 2842216 | | |
| NMTN-08447 | SAV2674 | | 2841989 | 2842693 | 2842334 | | |
| NMTN-12151 | SAV2675 | hisH | 2842686 | 2843264 | 2842824 | − | amidotransferase hisH |
| NMTN-11258 | SAV2675 | hisH | 2842686 | 2843264 | 2842888 | | |
| NMTN-11259 | SAV2675 | hisH | 2842686 | 2843264 | 2842891 | | |
| NMTN-02790 | SAV2675 | hisH | 2842686 | 2843264 | 2843041 | | |
| NMTN-08819 | SAV2675 | hisH | 2842686 | 2843264 | 2843041 | | |
| NMTN-05034 | SAV2675 | hisH | 2842686 | 2843264 | 2843059 | | |
| NMTN-00062 | SAV2676 | hisB | 2843261 | 2843839 | 2843458 | − | imidazoleglycerol-phosphate |
| | | | | | | | dehydratase |
| NMTN-06380 | SAV2676 | hisB | 2843261 | 2843839 | 2843518 | | |
| NMTN-02701 | SAV2676 | hisB | 2843261 | 2843839 | 2843561 | | |
| NMTN-13480d | SAV2676 | hisB | 2843261 | 2843839 | 2843564 | | |
| NMTN-13145 | SAV2676 | hisB | 2843261 | 2843839 | 2843670 | | |
| NMTN-02252 | SAV2677 | | 2843808 | 2844821 | 2843870 | − | hypothetical protein |
| NMTN-13560 | SAV2677 | | 2843808 | 2844821 | 2844127 | | |
| NMTN-14349 | SAV2677 | | 2843808 | 2844821 | 2844127 | | |
| NMTN-00731 | SAV2677 | | 2843808 | 2844821 | 2844164 | | |
| NMTN-10193 | SAV2677 | | 2843808 | 2844821 | 2844402 | | |
| NMTN-14028 | SAV2677 | | 2843808 | 2844821 | 2844760 | | |
| NMTN-00416 | SAV2678 | | 2844837 | 2846087 | 2844873 | − | hypothetical protein |
| NMTN-02448 | SAV2678 | | 2844837 | 2846087 | 2845323 | | |
| NMTN-02001 | SAV2678 | | 2844837 | 2846087 | 2845700 | | |
| NMTN-02009 | SAV2678 | | 2844837 | 2846087 | 2845700 | | |
| NMTN-01818 | SAV2679 | hisG | 2846080 | 2846694 | 2846359 | − | ATP phosphoribosyltransferase |
| NMTN-02348 | SAV2679 | hisG | 2846080 | 2846694 | 2846634 | | |
| NMTN-15276 | SAV2679 | hisG | 2846080 | 2846694 | 2846645 | | |
| NMTN-09578 | SAV2680 | | 2846712 | 2847530 | 2846760 | − | hypothetical protein |
| NMTN-03523 | SAV2680 | | 2846712 | 2847530 | 2847227 | | |
| NMTN-00619 | SAV2680 | | 2846712 | 2847530 | 2847311 | | |
| NMTN-14435 | SAV2681 | | 2847834 | 2848958 | 2848147 | − | hypothetical protein |
| NMTN-13874 | SAV2681 | | 2847834 | 2848958 | 2848647 | | |
| NMTN-01573 | SAV2681 | | 2847834 | 2848958 | 2848741 | | |
| NMTN-12301 | SAV2682 | | 2849076 | 2849573 | 2849483 | − | conserved hypothetical protein |
| NMTN-12301 | SAV2683 | | 2849076 | 2849573 | 2849483 | | |
| NMTN-06467 | SAV2683 | | 2849620 | 2850453 | 2849621 | − | conserved hypothetical protein |
| NMTN-02640 | SAV2684 | | 2850450 | 2852057 | 2850478 | − | hypothetical protein |
| NMTN-00726 | SAV2684 | | 2850450 | 2852057 | 2851213 | | |
| NMTN-00683 | SAV2685 | | 2852215 | 2852769 | 2852231 | − | conserved hypothetical protein |
| NMTN-01613 | SAV2685 | | 2852215 | 2852769 | 2852499 | | |
| NMTN-04657 | SAV2686 | | 2852798 | 2853646 | 2853239 | − | conserved hypothetical protein |
| NMTN-07854 | SAV2686 | | 2852798 | 2853646 | 2853341 | | |
| NMTN-00734 | SAV2686 | | 2852798 | 2853646 | 2853372 | | |
| NMTN-02732 | SAV2688 | drp35 | 2854693 | 2855667 | 2854835 | − | Drp35 |
| NMTN-12111 | SAV2688 | drp35 | 2854693 | 2855667 | 2854952 | | |
| NMTN-13419 | SAV2689 | | 2855908 | 2856864 | 2855937 | + | conserved hypothetical protein |
| NMTN-07083 | SAV2689 | | 2855908 | 2856864 | 2855990 | | |
| NMTN-05335 | SAV2689 | | 2855908 | 2856864 | 2856080 | | |
| NMTN-08989 | SAV2689 | | 2855908 | 2856864 | 2856169 | | |
| NMTN-12851 | SAV2689 | | 2855908 | 2856864 | 2856169 | | |
| NMTN-13869 | SAV2689 | | 2855908 | 2856864 | 2856638 | | |
| NMTN-00709 | SAV2689 | | 2855908 | 2856864 | 2856674 | | |
| NMTN-14602 | SAV2690 | pcp | 2857016 | 2857654 | 2857091 | + | pyrrolidone-carboxylate peptidase |
| NMTN-03122 | SAV2690 | pcp | 2857016 | 2857654 | 2857157 | | |
| NMTN-03122 | SAV2690 | pcp | 2857016 | 2857654 | 2857157 | | |
| NMTN-11060 | SAV2690 | pcp | 2857016 | 2857654 | 2857260 | | |
| NMTN-04754 | SAV2691 | | 2857773 | 2858540 | 2858313 | − | hypothetical protein |
| NMTN-09586 | SAV2691 | | 2857773 | 2858540 | 2858354 | | |
| NMTN-12749 | SAV2691 | | 2857773 | 2858540 | 2858409 | | |
| NMTN-09329 | SAV2693 | | 2859035 | 2859499 | 2859239 | + | hypothetical protein |
| NMTN-02592 | SAV2693 | | 2859035 | 2859499 | 2859422 | | |
| NMTN-02837 | SAV2694 | | 2859737 | 2860555 | 2860062 | + | 2-oxoglutarate/malate translocator |
| NMTN-09484 | SAV2696 | | 2861568 | 2862476 | 2861624 | − | hypothetical protein |
| NMTN-13670 | SAV2696 | | 2861568 | 2862476 | 2861669 | | |
| NMTN-11439 | SAV2696 | | 2861568 | 2862476 | 2861929 | | |
| NMTN-06096 | SAV2696 | | 2861568 | 2862476 | 2861998 | | |
| NMTN-04759 | SAV2696 | | 2861568 | 2862476 | 2862143 | | |
| NMTN-10026 | SAV2696 | | 2861568 | 2862476 | 2862150 | | |
| NMTN-14292 | SAV2697 | | 2862698 | 2863273 | 2862754 | + | hypothetical protein |
| NMTN-00295 | SAV2697 | | 2862698 | 2863273 | 2862890 | | |
| NMTN-03149 | SAV2697 | | 2862698 | 2863273 | 2863054 | | |
| NMTN-08648 | SAV2698 | | 2863435 | 2864451 | 2863477 | + | hypothetical protein |
| NMTN-03556 | SAV2698 | | 2863435 | 2864451 | 2863481 | | |
| NMTN-12053 | SAV2698 | | 2863435 | 2864451 | 2863484 | | |
| NMTN-12056 | SAV2698 | | 2863435 | 2864451 | 2863484 | | |
| NMTN-13695 | SAV2698 | | 2863435 | 2864451 | 2863579 | | |
| NMTN-12455 | SAV2698 | | 2863435 | 2864451 | 2863996 | | |
| NMTN-07874 | SAV2698 | | 2863435 | 2864451 | 2864023 | | |
| NMTN-09337 | SAV2699 | | 2864688 | 2865488 | 2865252 | + | hypothetical protein |
| NMTN-02821 | SAV2700 | | 2865622 | 2866134 | 2865887 | − | conserved hypothetical protein |
| NMTN-09820 | SAV2700 | | 2865622 | 2866134 | 2866091 | | |
| NMTN-11238 | SAV2701 | vraD | 2866417 | 2867175 | 2866810 | + | hypothetical protein |
| NMTN-07568 | SAV2701 | vraD | 2866417 | 2867175 | 2866826 | | |
| NMTN-03823 | SAV2701 | vraD | 2866417 | 2867175 | 2867118 | | |
| NMTN-07472 | SAV2702 | vraE | 2867165 | 2869045 | 2867235 | + | hypothetical protein |
| NMTN-06193 | SAV2702 | vraE | 2867165 | 2869045 | 2867568 | | |
| NMTN-09390 | SAV2702 | vraE | 2867165 | 2869045 | 2868328 | | |
| NMTN-02496 | SAV2702 | vraE | 2867165 | 2869045 | 2868479 | | |
| NMTN-05599 | SAV2702 | vraE | 2867165 | 2869045 | 2868643 | | |
| NMTN-03993 | SAV2702 | vraE | 2867165 | 2869045 | 2868841 | | |
| NMTN-12126 | SAV2705 | | 2870038 | 2870607 | 2870177 | − | hypothetical protein |
| NMTN-03720 | SAV2705 | | 2870038 | 2870607 | 2870180 | | |
| NMTN-09317 | SAV2705 | | 2870038 | 2870607 | 2870359 | | |
| NMTN-08451 | SAV2705 | | 2870038 | 2870607 | 2870420 | | |
| NMTN-01767 | SAV2705 | | 2870038 | 2870607 | 2870421 | | |
| NMTN-03436 | SAV2705 | | 2870038 | 2870607 | 2870533 | | |
| NMTN-13101 | SAV2705 | | 2870038 | 2870607 | 2870608 | | |
| NMTN-10895 | SAV2705 | | 2870038 | 2870607 | 2870612 | | |
| NMTN-14388 | SAV2706 | | 2870867 | 2871262 | 2870874 | − | hypothetical protein |
| NMTN-03664 | SAV2706 | | 2870867 | 2871262 | 2871132 | | |
| NMTN-02441 | SAV2707 | | 2871330 | 2871683 | 2871509 | − | hypothetical protein |
| NMTN-09932 | SAV2707 | | 2871330 | 2871683 | 2871577 | | |
| NMTN-13476 | SAV2704 | cspB | 2873016 | 2873735 | 2873161 | + | cold shock protein |
| NMTN-13476 | SAV2710 | gidB | 2873016 | 2873735 | 2873161 | − | glucose inhibited division protein B |
| NMTN-13747 | SAV2711 | gidA | 2873735 | 2875612 | 2873902 | − | glucose inhibited division protein A |
| NMTN-12375 | SAV2711 | gidA | 2873735 | 2875612 | 2874522 | | |
| NMTN-07232 | SAV2711 | gidA | 2873735 | 2875612 | 2874915 | | |
| NMTN-05453 | SAV2711 | gidA | 2873735 | 2875612 | 2874917 | | |
| NMTN-02858 | SAV2711 | gidA | 2873735 | 2875612 | 2875072 | | |
| NMTN-13310 | SAV2711 | gidA | 2873735 | 2875612 | 2875218 | | |
| NMTN-02246 | SAV2711 | gidA | 2873735 | 2875612 | 2875236 | | |
| NMTN-08904 | SAV2711 | gidA | 2873735 | 2875612 | 2875251 | | |
| NMTN-14505 | SAV2712 | thdF | 2875679 | 2877058 | 2875888 | − | possible thiophene and furan oxidation |
| | | | | | | | protein |
| NMTN-10847 | SAV2712 | thdF | 2875679 | 2877058 | 2876202 | | |
| NMTN-07508 | SAV2712 | thdF | 2875679 | 2877058 | 2876314 | | |
| NMTN-03570 | SAV2712 | thdF | 2875679 | 2877058 | 2876559 | | |
| NMTN-14721 | SAV2712 | thdF | 2875679 | 2877058 | 2876634 | | |
| NMTN-15315 | SAV0819 | | 896675 | 897244 | | | |
| NMTN-05384 | SAV2038 | agrC | | | | | |

### REFERENCES

The following references are specifically incorporated herein by reference.
U.S. Pat. No. 4,430,434
U.S. Pat. No. 4,559,302
U.S. Pat. No. 4,683,202
U.S. Pat. No. 4,727,028
U.S. Pat. No. 4,960,704
U.S. Pat. No. 5,928,906
Albus et al., Infect. Immun., 59:1008-1014, 1991.
Alksne Projan, Cur. Opin. Biotechnol., 11:625-636, 2000.
Archer and Climo, N. Engl. J. Med., 344:55-56, 2001.
Archer, Clin. Infect. Dis., 26:1179-1181, 1998.
Ausubel et al., In: Current Protocols in Molecular Biology, John, Wiley & Sons, Inc, New York, 1994.
Baba et al., Lancet, 359:1819-1827, 2002.
Brandenberger et al., Biochim. Biophys. Acta, 1523:135-139, 2000.
Brumfitt, W. & Hamilton-Miller, J. (1989) N. Engl. J. Med. 320, 1188-1199.
Carbonelli et al., FEMS Microbiol. Lett., 177(1):75-82, 1999.
Chalfie et al., Science, 263(5148):802-805, 1994.
Chang et al., N. Engl. J. Med., 348:1342-1347, 2003.
Cheung et al., Curr. Infect. Dis. Rep., 4:400-410, 2002.
Cocea, Biotechniques, 23(5):814-816, 1997.
Colbere-Garapin et al., Dev Biol Stand, 50:323-326, 1981.
Coulter et al., Mol. Microbiol., 30:393-404, 1998.
Dubrac and Msadek, J. Bacteriol., 186:1175-1181, 2004.
Duthie and Lorenz, J. Gen. Microbiol., 6:95-107, 1952.
Fechheimer, et al., Proc Natl. Acad. Sci. USA, 84:8463-8467, 1987.
Fitzgerald and Foster, J. Bacteriol., 182:1046-1052, 2000.
Fitzgerald et al., Proc. Natl. Acad. Sci. USA, 98:8821-8826, 2001.
Forsyth et al., Mol. Microbiol., 43:1387-1400, 2002.
Fournier et al., Mol. Microbiol., 41:247-261, 2001.
Garsin et al., Proc. Natl. Acad. Sci. USA, 98:10892-10897, 2001.
Gillet et al., Lancet, 359:753-759, 2002.
Giraudo et al., FEMS Microbiol. Lett., 177:15-22, 1999.
Götz, Mol. Microbiol. 43:1367-1378, 2002.
Harland and Weintraub, J. Cell Biol., 101(3):1094-1099, 1985.
Hartley and Paddon, Plasmid, 16:45-51, 1986.
Hiramatsu et al., J. Antimicrob. Chemother., 40:135-136, 1997.
Holden et al., Proc. Natl. Acad. Sci. USA, 101:9786-9791, 2004.
Janzon and Arvidson, EMBO J., 9:1391-1399, 1990.
Ji et al., Science, 293:2266-2269, 2001.
Kaufman et al., Biochem. Pharmacol., 40(12):2637-40, 1990.
Klein et al., Nature, 327:70-73, 1987.
Kobayashi et al., Proc. Natl. Acad. Sci. USA, 100:4678-4683, 2003.
Kohler et al., J. Bacteriol., 185:6928-6937, 2003.
Kullik et al., J. Bacteriol., 180:4814-4820, 1998.
Kuroda et al., Lancet, 357:1225-1240, 2001.
Lampe et al., EMBO J., 15:5470-5479, 1996.
Lee et al., J. Infect. Dis., 156:741-750, 1987.
Levenson et al., Hum. Gene Ther., 9(8):1233-1236, 1998.
Lewis and Fleming, In: Methods Cell Biol., Academic Press, New York, 48:4-27, 1995.
Lithgow et al., Microbiology, 150:373-381, 2004.
Lowe et al., Mol. Microbiol., 27:967-976, 1998.
Lowy, New Engl. J. Med., 339:520-532, 1998.
Mahan et al., Science, 259:686-688, 1993.
Mazmanian and Schneewind, In: Bacillus subtilis and its closest relatives, Sonenshine et al. (Eds.), ASM Press, NY, 57-70, 2002.
Mazmanian et al., Proc. Natl. Acad. Sci. USA, 97:5510-5515, 2000.
Mei et al, Mol. Microbiol., 26:399-407, 1997.
Neu, Science, 257:1064-1073, 1992.
Novick et al., EMBO J., 12:3967-3975, 1993.
Novick, Mol. Microbiol., 48:1429-1449, 2003.
Oliveira et al., Lancet Infect. Dis., 2:180-189, 2002.
Pragman et al., J. Bacteriol., 186:2430-2438, 2004.
Robertson and Lampe, Mol. Biol. Evol., 12:850-862, 1995.
Rossi et al., Antimicrob. Agents Chemother., 47:2558-2564, 2003.
Sambrook et al., In: Molecular cloning, Cold Spring Harbor Laboratory Press, Cold Spring Harbor, N.Y., 1989.
Santerre et al., Gene, 30: 147-156, 1984.
Sau et al., Microbiology, 143:2395-2405, 1997.
Schindler and Schuhardt, Proc. Natl. Acad. Sci. USA, 51:414-421. 1964.
Schwan et al., Infect. Immun., 66:567-572, 1998.
Sifri et al., Infect. Immun., 71:2208-2217, 2003.
Somerville et al., Infect. Immun., 71:4724-4732, 2003.
Somerville et al., J. Bacteriol., 184:1430-1437, 2002.
Trieu-Cuot et al., Nucl. Acids Res., 18:3660, 1990.
Vlach et al., Comp. Med., 50:160-166, 2000.
Walsh, Nature, 406:775-781, 2000.
Weigel et al., Science, 302:1569-1571, 2003.
Yang et al., Proc. Natl. Acad. Sci. USA, 87:9568-9572, 1990.

What is claimed is:

1. A transposable nucleic acid element comprising an selectable marker gene and a pair of inverted repeat sequences recognized by a Himar 1 transposase, wherein an inverted repeat sequence is toward each end of the element.

2. The transposable nucleic acid element of claim 1, wherein the inverted repeat sequences are SEQ ID NO:4 and SEQ ID NO:5.

3. The transposable nucleic acid element of claim 1, wherein the selectable marker gene encodes a polypeptide that confers antibiotic resistance.

4. The transposable nucleic acid element of claim 1, wherein the selectable marker gene encodes a polypeptide that confers resistance to erythromycin or tetracycline.

5. The transposable element of claim 1, further comprising a gene encoding a green fluorescent protein.

6. The transposable element of claim 1, wherein the element is in a plasmid.

7. The transposable element of claim 6, wherein the plasmid further comprises a temperature-sensitive replicon.

8. The transposable element of claim 1, wherein the plasmid is in a bacteria.

9. The transposable element of claim 8, wherein the bacteria is a gram positive bacteria.

10. The transposable element of claim 9, wherein the gram positive bacteria is a staphylococcus bacteria.

11. The transposable element of claim 8, wherein the bacteria further comprises a Himar 1 transposase-encoding nucleic acid.

12. The transposable element of claim 11, wherein the Himar 1 transposase-encoding nucleic acid is not contained in the plasmid with the transposable element.

13. The transposable element of claim 12, wherein the Himar 1 transposase-encoding nucleic acid is contained in a different plasmid than the plasmid with the transposable element.

14. The transposable element of claim 1, wherein the element is Bursa aurealis.

15. The transposable element of claim 14, wherein the element has the sequence of SEQ ID NO:3.

16. An Staphylococcus aureus Newman strain bacterium comprising a transposon insertion.

17. The bacterium of claim 16, wherein the transposon comprises a pair of inverted repeats recognized by a Himar 1 transposase.

18. The bacterium of claim 17, wherein the transposon further comprises a marker gene.

19. The bacterium of claim 18, wherein the marker gene is a selectable gene.

20. The bacterium of claim 19, wherein the selectable gene encoded a polypeptide that confers antibiotic resistance.

21. The bacterium of claim 17, wherein the transposon is Bursa aurealis.

22. The bacterium of claim 16, wherein the insertion is in a gene selected from the group consisting of: metabolic gene, regulatory gene, extracellular factor gene, cellular or secreted gene, conserved hypothetical gene, and hypothetical gene.

23. The bacterium of claim 16, wherein the insertion is in a gene encoding an RNA or protein selected from the group consisting of: rRNA, tRNA, enzyme, structural protein, transporter, symporter, and membrane protein.

24. The bacterium of claim 21, wherein the insertion negatively affects virulence.

25. The bacterium of claim 21, wherein the insertion positively affects virulence.

26. A method for generating one or more gram positive bacterial mutants comprising:
a) transfecting into a gram positive bacterium i) a nucleic acid comprising a transposable element comprising a screenable marker gene and a pair of inverted repeats recognized by a Himar 1 transposase at each end of the element and ii) a gene encoding a Himar 1 transposase, wherein the gene is under the control of a promoter that expresses the transposase in the bacterium;
b) screening the bacterium using the phenotype conferred by the screenable marker gene, wherein integration of the transposable element creates a genetic mutation.

27. The method of claim 26, wherein the screenable marker gene is a selectable marker gene and the screening involves selecting the bacterium using the phenotype conferred by the selectable marker gene

28. The method of claim 26, wherein the nucleic acid comprising the transposable element is a plasmid that also includes a temperature-sensitive gram positive replicon.

29. The method of claim 28, further comprising raising the temperature to a nonpermissive temperature of the replicon between steps a) and b).

30. The method of claim 27, wherein the bacterium is selected using antibiotic resistance conferred by a selectable marker encoding an antibiotic resistance polypeptide.

31. The method of claim 30, wherein the antibiotic is erythromycin.

32. The method of claim 26, further comprising identifying the location of the genetic mutation.

33. The method of claim 32, wherein identifying the location of the genetic mutation involves sequencing a region on one or both sides of the integrated transposable element.

34. The method of claim 26, further comprising identifying a transposon insertion mutation in a virulence gene.

35. The method of claim 34, wherein a transposon insertion mutation in a virulence gene is identified using a nematode killing assay.

36. The method of claim 35, further comprising infecting a mouse with a mutant having a mutation identified using the nematode killing assay and analyzing the mouse for organ abscesses.

37. A method of identifying a gram positive bacterium having a mutation in a putative virulence gene comprising:
a) obtaining a gram positive bacteria with a transposon insertion, wherein the transposon insertion comprises a pair of inverted repeats recognized by a Himar 1 transposase;
b) incubating the bacteria with nematodes on an agar plate; and,
c) assaying for nematode killing, wherein reduced nematode killing compared to nematode killing by gram positive bacteria without a transposon insertion identifies a bacterium with a mutation in a putative virulence gene.

38. The method of claim 37, further comprising identifying the putative virulence gene.

39. The method of claim 38, further comprising screening for an inhibitor of the identified putative virulence gene.

40. The method of claim 37, wherein a pool of gram positive bacteria with transposon insertions is incubated with nematodes on an agar plate, wherein the pool includes between 2 and 500 bacteria with different transposon insertions.

41. The method of claim 40, wherein the pool includes between 5 and 400 bacteria with different transposon insertions.

42. The method of claim 41, wherein the pool includes between 10 and 200 bacteria with different transposon insertions.

43. The method of claim 42, wherein the pool includes between 50 an 100 bacteria with different transposon insertions.

44. The method of claim 37, further comprising lysing the bacteria with a transposon insertion in a putative virulence gene; transducing the insertion into a gram positive bacterium lacking a transposon insertion; and, evaluating the transduced bacteria with a nematode killing assay, wherein reduced killing compared to gram positive bacteria without a transposon insertion is further indicative of an insertion in a putative virulence gene.

45. The method of claim 44, further comprising infecting a mouse with the bacteria having a transposon insertion in a putative virulence gene and assaying the mouse for organ abscesses, wherein a reduced amount of organ abscesses compared to a mouse incubated with gram positive bacteria without a transposon insertion is further indicative of an insertion in a putative virulence gene.

46. The method of claim 37, further comprising sequencing the insertion site.

47. A method of identifying or characterizing a virulence gene in a gram positive bacterium comprising:
a) incubating with nematodes on an agar plate a gram positive bacterium with a transposon insertion in a putative virulence gene, wherein the transposon insertion comprises a pair of inverted repeats recognized by a Himar 1 transposase; and,
b) assaying for nematode killing, wherein reduced nematode killing compared to nematode killing by gram positive bacteria without a transposon insertion is indicative of an insertion in a putative virulence gene.

48. A method of screening for a virulence gene in a gram positive bacteria comprising:
a) obtaining a gram positive bacteria with a transposon insertion, wherein the transposon insertion comprises a pair of inverted repeats recognized by a Himar 1 transposase;
b) infecting a mouse with the bacteria having a transposon insertion and assaying the mouse for organ abscesses, wherein a reduced amount of organ abscesses compared to a mouse incubated with the gram positive bacteria without a transposon insertion is indicative of an insertion in a putative virulence gene.

49. The method of claim 48, wherein the mouse is infected with a pool of gram positive bacteria having transposon insertions, wherein the pool includes between 2 and 10,000 bacteria with different transposon insertions.

50. A collection of S. aureas Newman mutants having a transposon insertion.
